(19) United States
(12) Patent Application Publication
(10) Pub. No.: US 2026/0174875 A1
(43) Pub. Date: Jun. 25, 2026

(54) TRI-AGONISTS OF THE GLP-1, GIP, AND AMYLIN RECEPTORS

(71) Applicant: Novo Nordisk A/S, Bagsvaerd (DK)

(72) Inventors: Thomas Kruse, Herlev (DK); Jesper F. Lau, Farum (DK); Camilla Kaas Frich, Stenloese (DK); Charlotte Stahl Madsen, Maaloev (DK); Christina Sophie Haxvig, Hilleroed (DK); Carsten Behrens, Koebenhavn N (DK)

(21) Appl. No.: 19/328,308

(22) Filed: Sep. 15, 2025

Related U.S. Application Data
(63) Continuation of application No. PCT/EP2024/084031, filed on Nov. 29, 2024.

(30) Foreign Application Priority Data
Nov. 30, 2023 (EP) 23213366.0

(51) Int. Cl. A61K 47/54; A61K 47/55; A61K 47/64; A61K 47/65; A61P 3/04; C07K 14/00

(52) U.S. Cl. CPC A61K 47/542; A61K 47/55; A61K 47/64; A61K 47/65; A61P 3/04; C07K 14/001

(57) ABSTRACT

The present invention relates to a GLP-1-/GIP-/amylin-receptor tri-agonist. This GLP-1-/GIP-/amylin-receptor tri-agonist may have improved pharmacokinetic properties and may be suitable for once weekly administration. The invention also relates to pharmaceutical compositions comprising such a GLP-1-/GIP-/amylin-receptor tri-agonist. The tri-agonist and pharmaceutical compositions comprising it may be used as a medicament for the treatment of subjects with overweight, obesity and associated co-morbidity.

### CROSS-REFERENCE TO RELATED APPLICATIONS

This application is a continuation of International Application PCT/EP2024/084031, filed Nov. 29, 2024, which claims priority to European Patent Application 23213366.0, filed Nov. 30, 2023, the contents of which are incorporated by reference in their entirety.

### TECHNICAL FIELD

GLP-1-/GIP-/amylin-receptor tri-agonists and compositions comprising such compound for use in medicine.

### INCORPORATION-BY-REFERENCE OF THE SEQUENCE LISTING

The instant application contains a Sequence Listing which has been submitted in XML format via the USPTO patent electronic filing system and is hereby incorporated by reference in its entirety. Said XML file, created on May 29, 2025, is named 230050US01.xml and is 397,539 bytes in size.

### BACKGROUND

Overweight and obesity are the abnormal or excessive accumulation of body fat that present a risk to an individual's overall health. The WHO considers body mass index (BMI) to be the most convenient population-level measure of overweight and obesity. In adults, a body mass index (BMI) greater than or equal to 25 is considered overweight, and a BMI of greater than or equal to 30 is considered obese. Obesity is further subclassified into class I (BMI 30-34.9), class II (BMI 35-39.9) and class III (BMI>40).

Obesity is a leading risk factor in a large number of serious conditions, including type 2 diabetes and its associated co-morbidities, and cardiovascular diseases such as heart disease and stroke, which are the leading causes of death worldwide. Obesity is now recognised by the World Health Organization (WHO) as an issue that has grown to epidemic proportion, even in children: in 2016, 1.9 billion adults worldwide were reported to have obesity; in 2019, 38.3 million children under the age of 5 worldwide were reported to have obesity. According to the WHO, 422 million people worldwide have diabetes, and 1.6 million deaths are directly attributed to diabetes each year. There is, therefore, a huge incentive for the individual, as well as society, to try to prevent and/or treat obesity.

When life-style modification such as diet and exercise alone do not suffice in reducing the body mass index (BMI) of an individual living with obesity to an acceptable level, treatment with pharmaceutical drugs such as liraglutide, orlistat and naltrexone-bupropion have been shown to cause some weight loss. Nonetheless, these weight losses are often not sustained and are too small for individuals with class II and class III obesity. In these cases, bariatric surgery has proven necessary. While bariatric surgery is currently the most effective treatment in terms of obtaining long-term weight loss, it is an invasive procedure associated with high risk to the patient and high cost. Therefore, an efficacious and minimally invasive treatment would be a significant improvement in the treatment of obesity.

GLP-1 is a 30 or 31-amino acid polypeptide that is synthesised and secreted from enteroendocrine L-cells. GLP-1 is an incretin hormone, decreasing blood sugar levels in a glucose-dependent manner by enhancing the secretion of insulin. Endogenous GLP-1 is rapidly degraded, primarily by dipeptidyl peptidase-4 (DPP-4), resulting in a half-life of <2 minutes.

Several marketed products containing a long-acting GLP-1 receptor agonist as the active pharmaceutical ingredient are approved for treatment of type 2 diabetes. These include dulaglutide (Trulicity®), exenatide (Byetta®, Bydureon®), liraglutide (Victoza®), lixisenatide (Lyxumia®), and semaglutide (Ozempic®).

Two marketed products containing a GLP-1 receptor agonist as the active pharmaceutical ingredient are approved for use in individuals living with overweight and have at least one weight-related co-morbidity or in individuals living with obesity: liraglutide (Saxenda®) and semaglutide (Wegovy®). The maximum efficacy that can be achieved with a GLP-1 receptor agonist is limited by tolerability. At increasing doses, side-effects such as nausea and vomiting become increasingly pronounced.

Native human GIP is a 42-amino acid polypeptide synthesized in and secreted by specialized enteroendocrine K-cells. These cells are concentrated primarily in the duodenum and proximal jejunum, although they also can be found throughout the intestine. The main stimulant for GIP secretion is ingestion of carbohydrate- and lipid-rich meals. Following ingestion, circulating plasma GIP levels increase 10- to 20-fold. Like GLP-1, GIP is an incretin hormone and in healthy humans it actually appears to be a more powerful incretin than GLP-1. However, in individuals living with type-2-diabetes GIP has lost its incretin effect. The half-life of intact GIP is estimated to be approximately 7 minutes in healthy subjects and approximately 5 minutes in people living with type-2 diabetes.

Long acting (or protracted) GIP analogues have been shown to lower body weight and improve glycaemic control. With regards to body weight reduction, this effect is comparatively less than long-acting GLP-1 analogues in rodent models (Mroz et al, Mol Metab, 2019, 20: 51-62). Moreover, GIP analogues induce body weight loss by an additive/synergistic action with long-acting GLP-1 analogues in dual administration (Finan et al, Sci Transl Med, 2013, 5 (209): 209ra151; Nørregaard et al, Diabetes Obes Metab, 2018, 20 (1): 60-68), and as such represent suitable candidates for amplification of GLP-1-based pharmacology. GIPR agonism can also be included as a partner to GLP-1 receptor agonism as a single molecule co-agonist to amplify GLP-1 driven body weight loss and improvement in glycaemic control, as has been shown in preclinical animal models (Finan et al, Sci Transl Med, 2013, 5 (209): 209ra151; Coskun et al, Mol Metab, 2018, 18: 3-14). Two different peptides (MAR709 and LY3298176, the latter known as tirzepatide) with high potency on both GLP-1R and GIPR have been tested in multi-dose clinical trial studies. The clinical results have demonstrated improvements in glycaemic control and body weight that exceed that achieved with comparable dosing of benchmark GLP-1 specific agonists (Frias et al, Cell Metab, 2017, 26 (2): 343-352; Frias et al, Lancet, 2018, 392 (10160): 2180-2193), demonstrating the translational aspects and therapeutic benefits of co-targeting GLP-1 and GIP receptors.

Lately this concept of co-targeting GLP-1 and GIP receptors with a GLP-1/GIP co-agonist has been proven, as the compound tirzepatide has been approved in 2022 for the treatment of diabetes. Furthermore, tirzepatide is also useful for the treatment of obesity, as it has shown in a phase 3 clinical trial that a high dose of tirzepatide (15 mg) helped patients to lose (mean) 20.9% of their body weight after 72 weeks of treatment, including a 20-week dose-escalation period (A M Jastreboff, L J Aronne, N N Ahmad, et al., N Engl J Med 2022; 387:205-216). Tirzepatide was recently approved for weight management in people with a BMI >30 or a BMI >27 and at least one weight related co-mobility (tradename: Zepbound®).

Beside tirzepatide, which is described in WO 2016/111971 A1, GLP-1/GIP co-agonists and their potential medical uses are described in several patent applications such as WO 2006/086769, WO 2010/011439, WO 2013/164483, WO 2014/192284, WO 2015/067715, WO 2015/022420, WO 2015/086728, WO 2015/086729, WO 2016/111971, WO 2020/023386, US 2014/162945, US 2014/357552, and WO 2022/018186.

Amylin is a 37-amino acid long polypeptide hormone produced in pancreatic beta (β)-cell from where it is co-secreted with insulin. Amylin has a half-life of 15-20 minutes. It produces its effects in several different organ systems, primarily acting via amylin receptors 1-3 (AMYR1-3). Amylin is an important regulator of energy metabolism in health and disease, inhibiting glucagon secretion, delaying gastric emptying, signalling satiety, and suppressing appetite. Other amylin actions have also been reported, such as on the cardiovascular system and on bone.

Clinical studies have shown that amylin receptor agonists may be useful for the treatment of overweight, obesity, type 1 diabetes and/or type 2 diabetes. Currently, there is one product on the market (Symlin®) which contains an amylin receptor agonist (pramlintide acetate) as the active pharmaceutical ingredient. Symlin®, a liquid pharmaceutical composition for subcutaneous administration, is approved for use in patients with type 1 or type 2 diabetes who use basal and mealtime insulin and have failed to achieve desired glycaemic control, despite optimal insulin therapy. Pramlintide for use in people living with overweight and obesity was also investigated. Pramlintide has a short half-life (less than 1 hour), necessitating administration thrice daily. Consequently, there is a large diurnal difference in the pramlintide plasma concentrations.

Amylin receptor agonist therapy is limited by tolerability in much the same way as GLP-1 receptor agonist therapy (and by similar side-effects such as nausea and vomiting). There has been a similar desire to be able to prolong the action of amylin and also the co-targeting of the amylin receptor and the GLP-1 receptor has been described. Amylin receptor agonists and their potential medical uses are described in several patent applications such as WO 2012/168432, WO 2016/034604, WO 2022/129254, WO 2022/063925, or US 2022/0288168

A fixed-dose combination of an amylin receptor agonist, cagrilintide, and a GLP-1 receptor agonist, semaglutide, is currently under investigation for the treatment of overweight and obesity (Lancet 2021; 397: 1736-48). The drug products being investigated are separate liquid pharmaceutical compositions for subcutaneous use. A clinical trial has demonstrated that a combination of cagrilintide and semaglutide induced greater weight loss in people living with obesity than the maximal approved dose of semaglutide monotherapy. Worsening of the side-effect profile was not observed. Co-agonists of GLP-1 and amylin receptors, and their potential medical uses are described in several patent applications such as WO 2022/129526 A1. Therein peptide co-agonists of the human GLP-1R and amylin receptor are disclosed, which are potent and are balanced, i.e., having a similar level of activation of both receptor systems, and which are showing oral bioavailability. Another example is WO 2007/022123, which describes hybrid polypeptides including exendin covalently linked to amylin. However, there is no co-agonist of GLP-1 and amylin receptors which has so far obtained market approval.

Finally, WO 2023/288313 and WO 2024/015922 disclose multi-agonist peptides useful as agents for the treatment and prevention of metabolic diseases and disorders, in particular diabetes and obesity. WO 2023/288313 discloses peptides including two or more component peptides including amylin, GIP, GLP-1, and/or calcitonin. The specifically disclosed peptides are tri-agonists of the GLP-1, GIP, and amylin receptors, showing activity on all three receptors and reduction of food intake and body weight in animals.

Whilst current therapy options and investigatory drugs provide promise, individuals living with overweight, obesity and/or associated co-morbidities can, at best, at current time hope to be treated with injectable pharmaceutical formulations or medications with some efficacy. There still remains a need in the art for a more efficacious medicament, which is potent in vitro and potent on weight loss, and which does not simultaneously result in a proportionally increased level of side-effects, and which has improved pharmacokinetic properties, has improved chemical stability, is suitable for once weekly dosing in humans, and/or is suitable for oral administration.

### SUMMARY

The present invention relates to a GLP-1-/GIP-/amylin-receptor tri-agonist comprising a peptide according to Formula I:

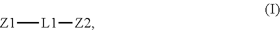

comprising one lysine (Lys, K) residue; wherein:
Z1 is a peptide comprising a maximum of 4 amino acid substitutions relative to Formula II (SEQ ID NO: 1):

(II)
YX₂EGTFTSDYSILLEEQAAREFIEWLLAGGPSKG,

wherein the amino acid at position X2 represents Aib;
L1 is a peptide linker; and
Z2 is a peptide comprising a C-terminal amide and a maximum of 10 amino acid substitutions relative to Formula III (SEQ ID NO: 2):

(III)
ASELSTAALGRLSAELHELATLPRTETGSGSP.

In another aspect the invention relates to a GLP-1-/GIP-/amylin-receptor tri-agonist comprising a peptide according to Formula I:

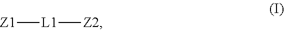

comprising one lysine (Lys, K) residue; wherein:
Z1 is a peptide having a maximum of 4 amino acid substitutions relative to Formula II (SEQ ID NO: 1):

(II)
YX₂EGTFTSDYSILLEEQAAREFIEWLLAGGPSKG,

wherein the amino acid at position X₂ represents Aib,
and wherein
Z1 comprises an amino acid sequence according to Formula X (SEQ ID NO: 161):

(X)
X₂₁X₂₂X₂₃GTFTSDYSX₂₄LLEEX₂₅AAX₂₆EFIX₂₇WLX₂₈X₂₉GGPSX₃₀X₃₁,

wherein
X₂₁ represents His (H) or Tyr (Y),
X₂₂ represents Aib,
X₂₃ represents Glu (E) or His (H),
X₂₄ represents Ile (I) or Lys (K),
X₂₅ represents Gln (Q) or Ile (I),
X₂₆ represents Arg (R) or Gln (Q),
X₂₇ represents Ala (A), Glu (E) or Gln (Q),
X₂₈ represents Leu (L) or I (Ile),
X₂₉ represents Ala (A) or Gln (Q),
X₃₀ represents Arg (R), Gly (G), Lys (K) or Ser (S),
X₃₁ represents Gly (G), Glu (E) or Lys (K);
   L1 is a peptide linker; and
   Z2 is a peptide comprising a C-terminal amide, and having a maximum of 10 amino acid substitutions relative to Formula III (SEQ ID NO: 2):

(III)
ASELSTAALGRLSAELHELATLPRTETGSGSP,

and
Z2 comprises an amino acid sequence according to Formula XII (SEQ ID NO:164):

(XII)
AX₃₂X₃₃LSTAX₃₄X₃₅X₃₆RLSAX₃₇LHX₃₈LX₃₉X₄₀X₄₁PX₄₂TETGSGX₄₃P,

wherein
X₃₂ represents Gly (G) or Ser (S),
X₃₃ represents Gln (Q), Glu (E), His (H) or Lys (K),
X₃₄ represents Ala (A) or Gln (Q),
X₃₅ represents Gln (Q), Leu (L) or Thr (T),
X₃₆ represents Ala (A), Gly (G) or Gln (Q),
X₃₇ represents Glu (E) or Lys (K),
X₃₈ represents Arg (R), Gln (Q), Glu (E), Gly (G), His (H), Lys (K), Thr (T) or Tyr (Y),
X₃₉ represents Ala (A) or Lys (K),
X₄₀ represents Asp (D) or Thr (T),
X₄₁ represents Leu (L) or Glu (E),
X₄₂ represents Arg (R) or Lys (K),
X₄₃ represents Ala (A) or Ser (S).

In another aspect the invention relates to a GLP-1-/GIP-/amylin-receptor tri-agonist comprising a peptide according to Formula I:

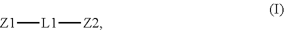

comprising one lysine (Lys, K) residue; wherein:
Z1 is a peptide having a maximum of 4 amino acid substitutions relative to Formula II (SEQ ID NO: 1):

(II)
YX₂EGTFTSDYSILLEEQAAREFIEWLLAGGPSKG,

wherein the amino acid at position X₂ represents Aib,
and wherein
Z1 comprises an amino acid sequence according to Formula IIa (SEQ ID NO: 3):

(IIa)
X₁X₂X₃GTFTSDYSILLEEQAAREFIEWLLAGGPSX₄X₅,

wherein
X₁ represents His (H) or Tyr (Y),
X₂ represents Aib,
X₃ represents Glu (E) or His (H),
X₄ represents Arg (R), Gly (G), Lys (K) or Ser (S),
X₅ represents Gly (G) or Lys (K);
   L1 is a peptide linker; and
   Z2 is a peptide comprising a C-terminal amide, and having a maximum of 10 amino acid substitutions relative to Formula III (SEQ ID NO: 2):

(III)
ASELSTAALGRLSAELHELATLPRTETGSGSP,

and
Z2 comprises an amino acid sequence according to Formula IIIa (SEQ ID NO: 4):

(IIIa)
ASX₆LSTAX₇X₈X₉RLSAX₁₀LHX₁₁LX₁₂X₁₃LPX₁₄TETGSGX₁₅P,

wherein
X₆ represents Gln (Q), Glu (E), His (H) or Lys (K),
X₇ represents Ala (A) or Gln (Q),
X₈ represents Gln (Q), Leu (L) or Thr (T),
X₉ represents Ala (A), Gly (G) or Gln (Q),
X₁₀ represents Glu (E) or Lys (K),
X₁₁ represents Arg (R), Gln (Q), Glu (E), Gly (G), His (H), Lys (K), Thr (T) or Tyr (Y),
X₁₂ represents Ala (A) or Lys (K),
X₁₃ represents Asp (D) or Thr (T),
X₁₄ represents Arg (R) or Lys (K),
X₁₅ represents Ala (A) or Ser (S).

In another aspect the invention relates to a GLP-1-/GIP-/amylin-receptor tri-agonist comprising a peptide according to Formula I:

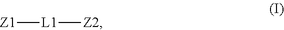

comprising one lysine (Lys, K) residue; wherein:
Z1 is a peptide having a maximum of 4 amino acid substitutions relative to Formula II (SEQ ID NO: 1):

(II)
YX₂EGTFTSDYSILLEEQAAREFIEWLLAGGPSKG,

wherein the amino acid at position X₂ represents Aib, and wherein
Z1 comprises an amino acid sequence according to Formula Xa (SEQ ID NO: 162):

(Xa)
YX₅₁EGTFTSDYSX₅₂LLEEIAAX₅₃EFIX₅₄WLX₅₅X₅₆GGPSSX₅₇,

wherein
X₅₁ represents Aib,
X₅₂ represents Ile (I) or Lys (K),
X₅₃ represents Arg (R) or Gln (Q),
X₅₄ represents Ala (A), Glu (E) or Gln (Q),
X₅₅ represents Leu (L) or I (Ile),
X₅₆ represents Ala (A) or Gln (Q),
X₅₇ represents Gly (G) or Glu (E);
   L1 is a peptide linker; and
   Z2 is a peptide comprising a C-terminal amide, and having a maximum of 10 amino acid substitutions relative to Formula III (SEQ ID NO: 2):

(III)
ASELSTAALGRLSAELHELATLPRTETGSGSP,

and
Z2 comprises an amino acid sequence according to Formula XIIa (SEQ ID NO: 165):

(XIIa)
AX₅₈X₅₉LSTAX₆₀X₆₁X₆₂RLSAELHX₆₃LATX₆₄PRTETGSGSP,

wherein
X₅₈ represents Gly (G) or Ser (S),
X₅₉ represents Gln (Q), Glu (E), or His (H),
X₆₀ represents Ala (A) or Gln (Q),
X₆₁ represents Leu (L) or Thr (T),
X₆₂ represents Ala (A), Gly (G) or Gln (Q),
X₆₃ represents Gln (Q), Glu (E), or Lys (K),
X₆₄ represents Leu (L) or Glu (E).

In some embodiments, the GLP-1-/GIP-/amylin-
receptor tri-agonist, has an amino acid
sequence which comprises or consists of
(SEQ ID NO: 62)
YXEGTFTSDYSILLEEQAAREFIEWLLAGGPSSGAGQAPGQAPGASHL
STAQTQRLSAELHKLATLPRTETGSGSP,
or
(SEQ ID NO: 65)
YXEGTFTSDYSILLEEQAAREFIEWLLAGGPSSGAGQAPGQAPGASHL
STAQTQRLSAKLHRLATLPRTETGSGSP,
or
(SEQ ID NO: 68)
YXEGTFTSDYSILLEEQAAREFIEWLLAGGPSSGAGQAPGQAPLASHL
STAQTQRLSAELHKLATLPRTETGSGSP,
or
(SEQ ID NO: 78)
YXEGTFTSDYSILLEEQAAREFIEWLLAGGPSRGEASHLSTAQTQRLS
AELHKLATLPRTETGSGSP,
or
(SEQ ID NO: 87)
YXEGTFTSDYSILLEEQAAREFIEWLLAGGPSSGAGQAPGQAPLASHL
STAQTQRLSAELHKLATLPRTETGSGSP,
or
(SEQ ID NO: 111)
HXHGTFTSDYSILLEEQAAREFIEWLLAGGPSKGAPPPSGGGEASHLS
TAQTARLSAELHQLATLPRTETGSGSP;
or
(SEQ ID NO: 170)
YXEGTFTSDYSILLEEIAAREFIEWLLAGGPSSGAGQAPGQAPGASHL
STAQTQRLSAELHKLATLPRTETGSGSP; wherein in each
amino acid sequence X represents Aib.

The GLP-1-/GIP-/amylin-receptor tri-agonist comprises a peptide linker L1, which comprises or consists of the amino acid sequence according to Formula IV:

(IV)
X₁X₂X₃X₄X₅X₆X₇X₈X₉X₁₀X₁₁X₁₂X₁₃X₁₄,

wherein
X₁ represents Ala (A), Glu (E), Gly (G),
X₂ represents Gln (Q), Glu (E), Gly (G), Leu (L), Pro (P) or is absent,
X₃ represents Ala (A), Gln (Q), Glu (E), Gly (G), Pro (P) or is absent,
X₄ represents Ala (A), Gln (Q), Glu (E), Gly (G), Pro (P) or is absent,
X₅ represents Glu (E), Gly (G), Pro (P), Ser (S), Thr (T) or is absent,
X₆ represents Glu (E), Gly (G), Leu (L), Gln (Q) or is absent,
X₇ represents Ala (A), Gln (Q), Glu (E), Gly (G), Phe (F) or is absent,
X₈ represents Ala (A), Gln (Q), Glu (E), Gly (G), Thr (T), Pro (P), Val (V) or is absent,
X₉ represents Glu (E), Asn (N), Pro (P), Thr (T) or is absent,
X₁₀ represents Ala (A), Gln (Q), Glu (E), Gly (G), Leu (L), Pro (P), Ser (S), Val (V) or is absent,
X₁₁ represents Ala (A) or is absent,
X₁₂ represents Gln (Q) or is absent,
X₁₃ represents Thr (T) or is absent,
X₁₄ represents Leu (L) or is absent.

In some embodiments, said GLP-1-/GIP-/amylin-receptor tri-agonist comprises a protraction moiety allowing for extended half-life.

Preferred GLP-1-/GIP-/amylin-receptor tri-agonists of the present invention are:

compound 52, that is:

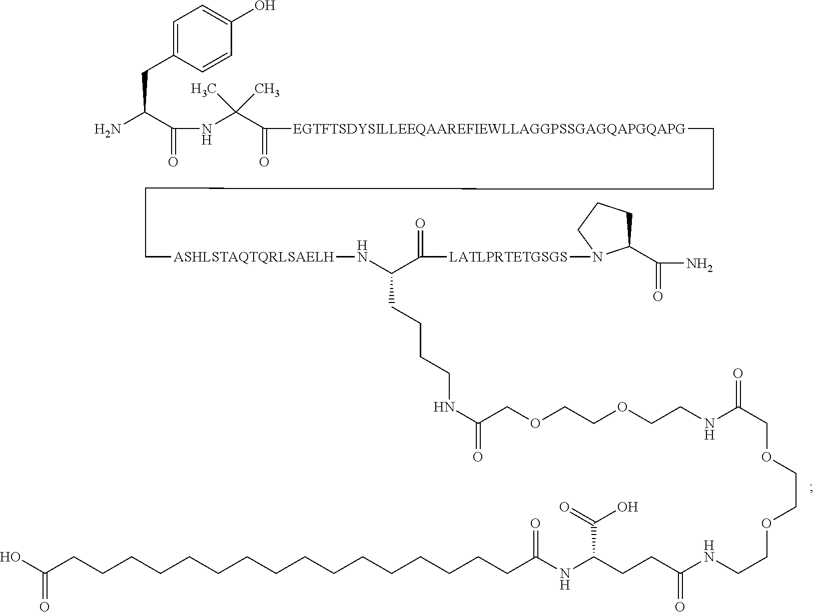

compound 55, that is:

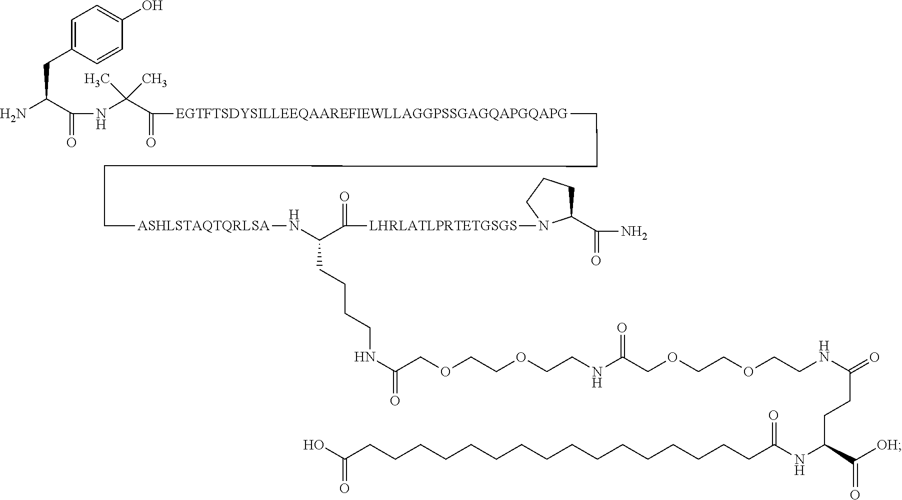

compound 58, that is:

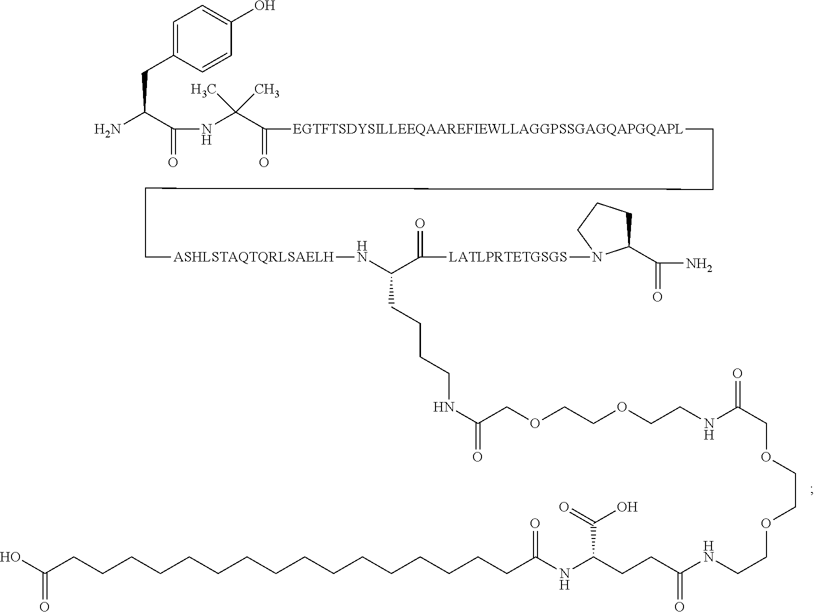

compound 68, that is:

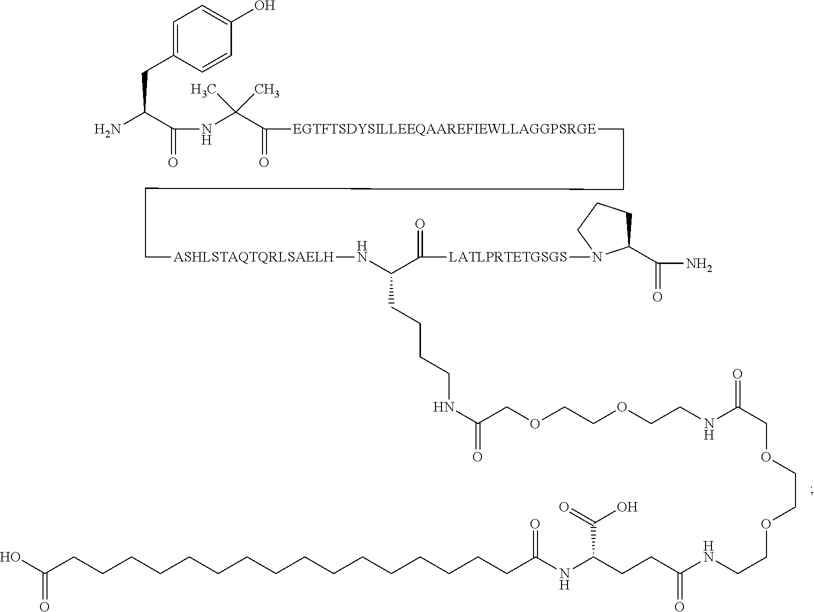

compound 77, that is:

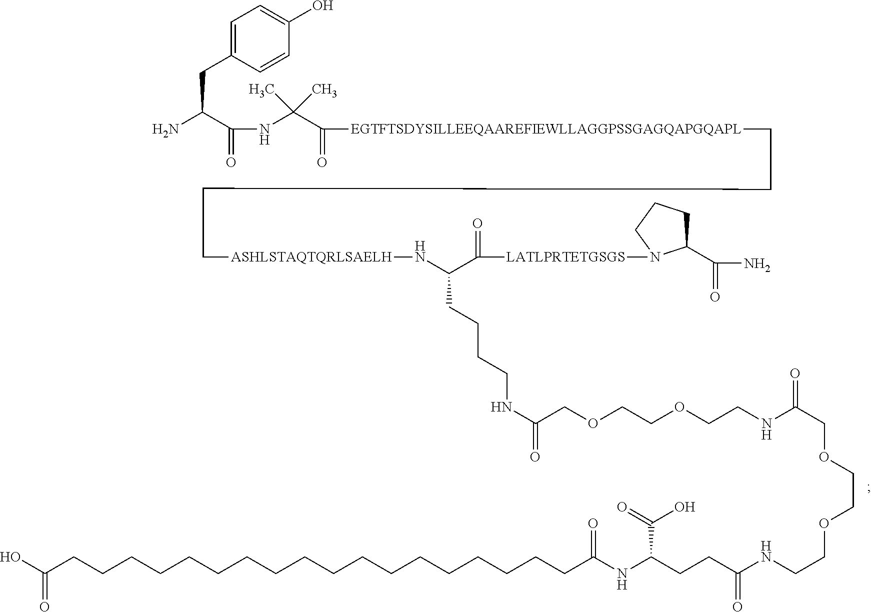

and
compound 101, that is:

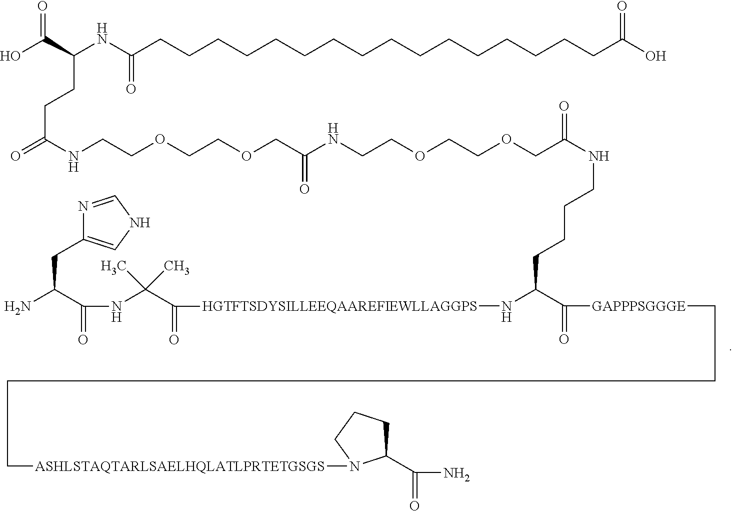

In a third aspect the invention relates to a balanced GLP-1-/GIP-/amylin-receptor tri-agonist that is capable of selectively activating or “agonising” all three of the GLP-1 receptor, the GIP receptor, and the amylin receptor to a similar level.

Also or alternatively, in a fourth aspect, the invention relates to a GLP-1-/GIP-/amylin-receptor tri-agonist with improved pharmacokinetic properties.

Also or alternatively, in a fifth aspect, the invention relates to a GLP-1-/GIP-/amylin-receptor tri-agonist which is suitable for once weekly administration.

Also or alternatively, in a sixth aspect, the invention relates to a GLP-1-/GIP-/amylin-receptor tri-agonist which is suitable for oral administration.

Also or alternatively, in a seventh aspect, the invention relates to a GLP-1-/GIP-/amylin-receptor tri-agonist with improved chemical stability.

Also, in further aspects the present invention relates to pharmaceutical compositions comprising such GLP-1-/GIP-/amylin-receptor triple agonist and one or more pharmaceutically acceptable excipients, as well as a GLP-1-/GIP-/amylin-receptor triple agonist for use as a medicament, in particularly for use in the treatment of subjects with an initial body mass index (BMI) of 27 or more, such as 30 or more, optionally in the presence of at least one weight-related co-morbidity.

The invention may also solve further problems that will be apparent from the disclosure of the exemplary embodiments and aspects.

### BRIEF DESCRIPTION OF DRAWINGS

FIG. 1 shows changes in body weight (in %) over time in a study in DIO rats, as described in Example 8, comparing vehicle ( [symbol] ); Compound 52 with a dose of 3 nmol/kg ( [symbol] ); Compound 52 with a dose of 10 nmol/kg ( [symbol] ); Compound 77 with a dose of 3 nmol/kg (-x-); and Compound 77 with a dose of 10 nmol/kg ( [symbol] ). DIO rats received a subcutaneous dose once daily following the titration schedule described in Table 14.

FIG. 2 shows daily food intake (in kcal) at baseline (day −4 to day 0) and during treatment (day 0-28) in a study in DIO rats, as described in Example 8, comparing vehicle ( [symbol] ); Compound 52 with a dose of 3 nmol/kg ( [symbol] ); Compound 52 with a dose of 10 nmol/kg ( [symbol] ); Compound 77 with a dose of 3 nmol/kg (-x-); and Compound 77 with a dose of 10 nmol/kg ( [symbol] ). DIO rats received a subcutaneous dose once daily following the titration schedule described in Table 14.

FIG. 3 shows cumulative food intake over time in a study in DIO rats, as described in Example 8a, comparing vehicle ( [symbol] ); Compound 52 with a dose of 3 nmol/kg ( [symbol] ); Compound 52 with a dose of 10 nmol/kg ( [symbol] ); Compound 77 with a dose of 3 nmol/kg (-x-); and Compound 77 with a dose of 10 nmol/kg ( [symbol] ). DIO rats received a subcutaneous dose once daily following the titration schedule described in Table 14.

### SEQUENCE LISTING

The present application is filed with a Sequence Listing in electronic form. The entire content of the sequence listing is hereby incorporated by reference.

SEQ ID NO: 1 represents the amino acid sequence of Formula II of Z1.

SEQ ID NO: 2 represents the amino acid sequence of Formula III of Z2.

SEQ ID NO: 3 represents the amino acid sequence of Formula IIa of Z1.

SEQ ID NO: 4 represents the amino acid sequence of Formula IIIa of Z2.

SEQ ID NO: 5 represents the most encompassing amino acid sequence of Formula I Z1-L1-Z2.

SEQ ID NO: 6 represents the amino acid sequence of Formula V of Z1.

SEQ ID NO: 7 represents the amino acid sequence of Formula VI of Z1.

SEQ ID NO: 8 represents the amino acid sequence of Formula VII of Z2.

SEQ ID NO: 9 represents the amino acid sequence of Formula VIII of Z2.

SEQ ID NO: 11 represents the amino acid sequence of the peptide backbone within reference compound 1.

SEQ ID NO: 12 represents the amino acid sequence of the peptide backbone within reference compound 2.

SEQ ID NO: 13 represents the amino acid sequence of the peptide backbone within reference compound 3.

SEQ ID NO: 14 represents the amino acid sequence of the peptide backbone within reference compound 4.

SEQ ID NO: 15 represents the amino acid sequence of the peptide backbone within reference compound 5 (tirzepatide).

SEQ ID NO: 16 represents the amino acid sequence of the peptide backbone within reference compound 6 (cagrilintide).

SEQ ID NO: 17 represents the amino acid sequence of the peptide backbone within reference compound 7 (semaglutide).

SEQ ID NOs: 20-124 represent the amino acid sequences of the peptide backbones in compounds 10 to 115 and 210.

SEQ ID NOs: 125-159 represent the amino acid sequences of exemplified peptide linkers L1.

SEQ ID NO: 161 represents the amino acid sequence of Formula X of Z1.

SEQ ID NO: 162 represents the amino acid sequence of Formula Xa of Z1.

SEQ ID NO: 163 represents the amino acid sequence of Formula XI of Z1.

SEQ ID NO: 164 represents the amino acid sequence of Formula XII of Z2.

SEQ ID NO: 165 represents the amino acid sequence of Formula XIIa of Z2.

SEQ ID NO: 166 represents the amino acid sequence of Formula XIII of Z2.

SEQ ID NOs: 170-242 represent the amino acid sequences of the peptide backbones in compounds 120 to 197 and 211 to 221.

TABLE 1
Amino acid sequences of the peptide backbone within compounds of the invention

| | SEQ ID | |
|---|---|---|
| Compound no. | NO: | Amino acid sequences |
| peptide backbone | 20 | YXEGTFTSDYSILLEEQAAREFIEWLLAGGPSKGAPPPSG |
| within compound | | GGEASELSTAALGRLSAELHELATLPRTETGSGSP |
| no. 10 | | |
| peptide backbone | 21 | YXEGTFTSDYSILLEEQAAREFIEWLLAGGPSKGEASELST |
| within compound | | AALGRLSAELHELATLPRTETGSGSP |
| no. 11 | | |
| peptide backbone | 22 | HXHGTFTSDYSILLEEQAAREFIEWLLAGGPSKGAPPPSG |
| within compound | | GGEASELSTAALGRLSAELHELATLPRTETGSGSP |
| no. 12 | | |
| peptide backbone | 23 | YXEGTFTSDYSILLEEQAAREFIEWLLAGGPSKGAPPPSG |
| within compound | | GGGASELSTAALGRLSAELHRLATLPRTETGSGSP |
| no. 13 | | |
| peptide backbone | 24 | HXHGTFTSDYSILLEEQAAREFIEWLLAGGPSKGAPPPSG |
| within compound | | GGGASELSTAALGRLSAELHRLATLPRTETGSGSP |
| no. 14 | | |
| peptide backbone | 25 | HXHGTFTSDYSILLEEQAAREFIEWLLAGGPSKGAPPPSG |
| within compound | | GGEASELSTAALGRLSAELHELATLPRTETGSGSP |
| no. 15 | | |
| peptide backbone | 26 | HXHGTFTSDYSILLEEQAAREFIEWLLAGGPSKGAPPPSG |
| within compound | | GGEASELSTAALGRLSAELHELATLPRTETGSGSP |
| no. 16 | | |
| peptide backbone | 27 | HXHGTFTSDYSILLEEQAAREFIEWLLAGGPSKGEASELS |
| within compound | | TAALGRLSAELHRLATLPRTETGSGSP |
| no. 17 | | |
| peptide backbone | 28 | YXEGTFTSDYSILLEEQAAREFIEWLLAGGPSKGEASELST |
| within compound | | AALGRLSAELHRLATLPRTETGSGSP |
| no. 18 | | |
| peptide backbone | 29 | YXEGTFTSDYSILLEEQAAREFIEWLLAGGPSKGEASELST |
| within compound | | AALGRLSAELHRLATLPRTETGSGSP |
| no. 19 | | |
| peptide backbone | 30 | YXEGTFTSDYSILLEEQAAREFIEWLLAGGPSKGASELSTA |
| within compound | | ALGRLSAELHRLATLPRTETGSGSP |
| no. 20 | | |
| peptide backbone | 31 | YXEGTFTSDYSILLEEQAAREFIEWLLAGGPSKGALAQTLA |
| within compound | | QTLASELSTAALGRLSAELHELATLPRTETGSGSP |
| no. 21 | | |
| peptide backbone | 32 | HXHGTFTSDYSILLEEQAAREFIEWLLAGGPSKGAPPPSL |
| within compound | | AQTLAQTLASELSTAALGRLSAELHELATLPRTETGSGSP |
| no. 22 | | |
| peptide backbone | 33 | YXEGTFTSDYSILLEEQAAREFIEWLLAGGPSKGALAQTL |
| within compound | | GTNEASELSTAALGRLSAELHELATLPRTETGSGSP |
| no. 23 | | |
| peptide backbone | 34 | YXEGTFTSDYSILLEEQAAREFIEWLLAGGPSKGALAQTLF |
| within compound | | VNQASELSTAALGRLSAELHELATLPRTETGSGSP |
| no. 24 | | |
| peptide backbone | 35 | HXHGTFTSDYSILLEEQAAREFIEWLLAGGPSKGEASELS |
| within compound | | TAALGRLSAELHRLATLPRTETGSGSP |
| no. 25 | | |
| peptide backbone | 36 | YXEGTFTSDYSILLEEQAAREFIEWLLAGGPSKGEASELST |
| within compound | | AALGRLSAELHELATLPRTETGSGSP |
| no. 26 | | |
| peptide backbone | 37 | YXEGTFTSDYSILLEEQAAREFIEWLLAGGPSKGEASELST |
| within compound | | AQLGRLSAELHRLATLPRTETGSGSP |
| no. 27 | | |
| peptide backbone | 38 | YXEGTFTSDYSILLEEQAAREFIEWLLAGGPSKGEASELST |
| within compound | | AALARLSAELHRLATLPRTETGSGSP |
| no. 28 | | |
| peptide backbone | 39 | YXEGTFTSDYSILLEEQAAREFIEWLLAGGPSKGEASELST |
| within compound | | AALGRLSAELHYLATLPRTETGSGSP |
| no. 29 | | |
| peptide backbone | 40 | HXHGTFTSDYSILLEEQAAREFIEWLLAGGPSKGAPPPSG |
| within compound | | GGEASELSTAALGRLSAELHRLATLPRTETGSGSP |
| no. 30 | | |
| peptide backbone | 41 | YXEGTFTSDYSILLEEQAAREFIEWLLAGGPSSKAPPPSG |
| within compound | | GGEASELSTAALGRLSAELHRLATLPRTETGSGSP |
| no. 31 | | |
| peptide backbone | 42 | YXEGTFTSDYSILLEEQAAREFIEWLLAGGPSKGAEASEL |
| within compound | | STAALGRLSAELHELATLPRTETGSGSP |
| no. 32 | | |
| peptide backbone | 43 | YXEGTFTSDYSILLEEQAAREFIEWLLAGGPSKGAPPEAS |
| within compound | | ELSTAALGRLSAELHELATLPRTETGSGSP |
| no. 33 | | |
| peptide backbone | 44 | YXEGTFTSDYSILLEEQAAREFIEWLLAGGPSKGEASHLS |
| within compound | | TAQTQRLSAELHELATLPRTETGSGSP |
| no. 34 | | |
| peptide backbone | 45 | YXEGTFTSDYSILLEEQAAREFIEWLLAGGPSKGEASHLS |
| within compound | | TAQTARLSAELHELATLPRTETGSGSP |
| no. 35 | | |
| peptide backbone | 46 | HXHGTFTSDYSILLEEQAAREFIEWLLAGGPSKGEASHLS |
| within compound | | TAQTARLSAELHELATLPRTETGSGSP |
| no. 36 | | |
| peptide backbone | 47 | HXHGTFTSDYSILLEEQAAREFIEWLLAGGPSKGAPPPSG |
| within compound | | GGEASHLSTAQTARLSAELHRLADLPRTETGSGSP |
| no. 37 | | |
| peptide backbone | 48 | YXEGTFTSDYSILLEEQAAREFIEWLLAGGPSKGGQAPGQ |
| within compound | | APGEASHLSTAQTQRLSAELHELATLPRTETGSGSP |
| no. 38 | | |
| peptide backbone | 49 | YXEGTFTSDYSILLEEQAAREFIEWLLAGGPSKGGQEPGQ |
| within compound | | EPGEASHLSTAQTQRLSAELHELATLPRTETGSGSP |
| no. 39 | | |
| peptide backbone | 50 | YXEGTFTSDYSILLEEQAAREFIEWLLAGGPSKGGGGEG |
| within compound | | GGEGEASHLSTAQTQRLSAELHELATLPRTETGSGSP |
| no. 40 | | |
| peptide backbone | 51 | YXEGTFTSDYSILLEEQAAREFIEWLLAGGPSKGAPPPSG |
| within compound | | GGEASELSTAALGRLSAELHRLADLPRTETGSGSP |
| no. 41 | | |
| peptide backbone | 52 | YXEGTFTSDYSILLEEQAAREFIEWLLAGGPSSKAPPPSG |
| within compound | | GGEASELSTAALGRLSAELHRLATLPRTETGSGSP |
| no. 42 | | |
| peptide backbone | 53 | HXHGTFTSDYSILLEEQAAREFIEWLLAGGPSKGEASQLS |
| within compound | | TAALGRLSAELHRLADLPRTETGSGSP |
| no. 43 | | |
| peptide backbone | 54 | HXHGTFTSDYSILLEEQAAREFIEWLLAGGPSKGALAQTL |
| within compound | | GTNEASELSTAALGRLSAELHRLATLPRTETGSGSP |
| no. 44 | | |
| peptide backbone | 55 | HXHGTFTSDYSILLEEQAAREFIEWLLAGGPSKGALAQTL |
| within compound | | GTNEASHLSTAQTQRLSAELHELATLPRTETGSGSP |
| no. 45 | | |
| peptide backbone | 56 | HXHGTFTSDYSILLEEQAAREFIEWLLAGGPSKGAGQAPG |
| within compound | | QAPGASHLSTAALGRLSAELHRLATLPRTETGSGSP |
| no. 46 | | |
| peptide backbone | 57 | HXHGTFTSDYSILLEEQAAREFIEWLLAGGPSKGAGQAPG |
| within compound | | QAPGASHLSTAQTQRLSAELHELATLPRTETGSGSP |
| no. 47 | | |
| peptide backbone | 58 | YXEGTFTSDYSILLEEQAAREFIEWLLAGGPSSGALAQTLA |
| within compound | | QTLASHLSTAQTQRLSAELHKLATLPRTETGSGSP |
| no. 48 | | |
| peptide backbone | 59 | YXEGTFTSDYSILLEEQAAREFIEWLLAGGPSSGAPPPSG |
| within compound | | GGEASHLSTAQTQRLSAELHKLATLPRTETGSGSP |
| no. 49 | | |
| peptide backbone | 60 | YXEGTFTSDYSILLEEQAAREFIEWLLAGGPSSGEASHLS |
| within compound | | TAQTQRLSAELHKLATLPRTETGSGSP |
| no. 50 | | |
| peptide backbone | 61 | HXHGTFTSDYSILLEEQAAREFIEWLLAGGPSSGALAQTL |
| within compound | | AQTLASHLSTAQTQRLSAELHKLATLPRTETGSGSP |
| no. 51 | | |
| peptide backbone | 62 | YXEGTFTSDYSILLEEQAAREFIEWLLAGGPSSGAGQAPG |
| within compounds | | QAPGASHLSTAQTQRLSAELHKLATLPRTETGSGSP |
| 52, 115, 210 | | |
| peptide backbone | 63 | YXEGTFTSDYSILLEEQAAREFIEWLLAGGPSSGAGQAPG |
| within compound | | QAPGASHLSTAALGRLSAELHKLATLPRTETGSGSP |
| no. 53 | | |
| peptide backbone | 64 | YXEGTFTSDYSILLEEQAAREFIEWLLAGGPSSGAGQAPG |
| within compound | | QAPGASKLSTAQTQRLSAELHRLATLPRTETGSGSP |
| no. 54 | | |
| peptide backbone | 65 | YXEGTFTSDYSILLEEQAAREFIEWLLAGGPSSGAGQAPG |
| within compound | | QAPGASHLSTAQTQRLSAKLHRLATLPRTETGSGSP |
| no. 55 | | |
| peptide backbone | 66 | YXEGTFTSDYSILLEEQAAREFIEWLLAGGPSSGAGQAPG |
| within compound | | QAPGASHLSTAQTQRLSAELHRLKTLPRTETGSGSP |
| no. 56 | | |
| peptide backbone | 67 | YXEGTFTSDYSILLEEQAAREFIEWLLAGGPSSGAGQAPG |
| within compound | | QAPGASHLSTAQTQRLSAELHRLATLPKTETGSGSP |
| no. 57 | | |
| peptide backbone | 68 | YXEGTFTSDYSILLEEQAAREFIEWLLAGGPSSGAGQAPG |
| within compound | | QAPLASHLSTAQTQRLSAELHKLATLPRTETGSGSP |
| no. 58 | | |
| peptide backbone | 69 | YXEGTFTSDYSILLEEQAAREFIEWLLAGGPSSGALAQTLA |
| within compound | | QTLASHLSTAQTQRLSAELHKLATLPRTETGSGSP |
| no. 59 | | |
| peptide backbone | 70 | YXEGTFTSDYSILLEEQAAREFIEWLLAGGPSSGALAQTLA |
| within compound | | QTLASHLSTAQTQRLSAELHKLATLPRTETGSGSP |
| no. 60 | | |
| peptide backbone | 71 | HXHGTFTSDYSILLEEQAAREFIEWLLAGGPSKGAPPPSG |
| within compound | | GGEASHLSTAQTARLSAELHRLADLPRTETGSGSP |
| no. 61 | | |
| peptide backbone | 72 | HXHGTFTSDYSILLEEQAAREFIEWLLAGGPSKGEASQLS |
| within compound | | TAALGRLSAELHRLADLPRTETGSGSP |
| no. 62 | | |
| peptide backbone | 73 | HXHGTFTSDYSILLEEQAAREFIEWLLAGGPSKGEASQLS |
| within compound | | TAALGRLSAELHTLATLPRTETGSGSP |
| no. 63 | | |
| peptide backbone | 74 | HXHGTFTSDYSILLEEQAAREFIEWLLAGGPSKGEASQLS |
| within compound | | TAALGRLSAELHQLATLPRTETGSGSP |
| no. 64 | | |
| peptide backbone | 75 | HXHGTFTSDYSILLEEQAAREFIEWLLAGGPSKGEASQLS |
| within compound | | TAALGRLSAELHGLATLPRTETGSGSP |
| no. 65 | | |
| peptide backbone | 76 | HXHGTFTSDYSILLEEQAAREFIEWLLAGGPSKGEASQLS |
| within compound | | TAALGRLSAELHHLATLPRTETGSGSP |
| no. 66 | | |
| peptide backbone | 77 | YXEGTFTSDYSILLEEQAAREFIEWLLAGGPSRGEASELS |
| within compound | | TAALGRLSAELHKLATLPRTETGSGSP |
| no. 67 | | |
| peptide backbone | 78 | YXEGTFTSDYSILLEEQAAREFIEWLLAGGPSRGEASHLS |
| within compound | | TAQTQRLSAELHKLATLPRTETGSGSP |
| no. 68 | | |
| peptide backbone | 79 | HXHGTFTSDYSILLEEQAAREFIEWLLAGGPSRGEASHLS |
| within compound | | TAQTQRLSAELHKLATLPRTETGSGSP |
| no. 69 | | |
| peptide backbone | 80 | HXHGTFTSDYSILLEEQAAREFIEWLLAGGPSRGAPPPSG |
| within compound | | GGEASELSTAALGRLSAELHKLATLPRTETGSGSP |
| no. 70 | | |
| peptide backbone | 81 | HXHGTFTSDYSILLEEQAAREFIEWLLAGGPSRGAPPPSG |
| within compound | | GGEASHLSTQTQGRLSAELHKLATLPRTETGSGSP |
| no. 71 | | |
| peptide backbone | 82 | YXEGTFTSDYSILLEEQAAREFIEWLLAGGPSRGAPPPSG |
| within compound | | GGEASHLSTQTQGRLSAELHKLATLPRTETGSGSP |
| no. 72 | | |
| peptide backbone | 83 | HXHGTFTSDYSILLEEQAAREFIEWLLAGGPSKGEASQLS |
| within compound | | TAALGRLSAELHRLATLPRTETGSGSP |
| no. 73 | | |
| peptide backbone | 84 | YXEGTFTSDYSILLEEQAAREFIEWLLAGGPSGGEASHLS |
| within compound | | TAQTQRLSAELHKLATLPRTETGSGSP |
| no. 74 | | |
| peptide backbone | 85 | HXHGTFTSDYSILLEEQAAREFIEWLLAGGPSGGEASHLS |
| within compound | | TAQTQRLSAELHKLATLPRTETGSGSP |
| no. 75 | | |
| peptide backbone | 86 | YXEGTFTSDYSILLEEQAAREFIEWLLAGGPSSGAGQAPG |
| within compound | | QAPGASHLSTAQTQRLSAELHRLATLPKTETGSGSP |
| no. 76 | | |
| peptide backbone | 87 | YXEGTFTSDYSILLEEQAAREFIEWLLAGGPSSGAGQAPG |
| within compound | | QAPLASHLSTAQTQRLSAELHKLATLPRTETGSGSP |
| no. 77 | | |
| peptide backbone | 88 | HXHGTFTSDYSILLEEQAAREFIEWLLAGGPSSGAGQAPG |
| within compound | | QAPGASHLSTAQTQRLSAKLHRLATLPRTETGSGSP |
| no. 78 | | |
| peptide backbone | 89 | YXEGTFTSDYSILLEEQAAREFIEWLLAGGPSSGEASHLS |
| within compound | | TAQTQRLSAKLHRLATLPRTETGSGSP |
| no. 79 | | |
| peptide backbone | 90 | YXEGTFTSDYSILLEEQAAREFIEWLLAGGPSSGEASHLS |
| within compound | | TAQTQRLSAELHRLKTLPRTETGSGSP |
| no. 80 | | |
| peptide backbone | 91 | YXEGTFTSDYSILLEEQAAREFIEWLLAGGPSSGEASHLS |
| within compound | | TAQTQRLSAELHRLATLPKTETGSGSP |
| no. 81 | | |
| peptide backbone | 92 | HXHGTFTSDYSILLEEQAAREFIEWLLAGGPSSGEASHLS |
| within compound | | TAQTQRLSAKLHRLATLPRTETGSGSP |
| no. 82 | | |
| peptide backbone | 93 | HXHGTFTSDYSILLEEQAAREFIEWLLAGGPSSGEASHLS |
| within compound | | TAQTQRLSAELHRLKTLPRTETGSGSP |
| no. 83 | | |
| peptide backbone | 94 | HXHGTFTSDYSILLEEQAAREFIEWLLAGGPSSGEASHLS |
| within compound | | TAQTQRLSAELHRLATLPKTETGSGSP |
| no. 84 | | |
| peptide backbone | 95 | HXHGTFTSDYSILLEEQAAREFIEWLLAGGPSSGEASHLS |
| within compound | | TAQTQRLSAELHKLATLPRTETGSGAP |
| no. 85 | | |
| peptide backbone | 96 | HXHGTFTSDYSILLEEQAAREFIEWLLAGGPSKGAPPPSG |
| within compound | | GGASELSTAALGRLSAELHRLATLPRTETGSGSP |
| no. 86 | | |
| peptide backbone | 97 | HXHGTFTSDYSILLEEQAAREFIEWLLAGGPSSGEASHLS |
| within compound | | TAALGRLSAKLHRLATLPRTETGSGSP |
| no. 87 | | |
| peptide backbone | 98 | HXHGTFTSDYSILLEEQAAREFIEWLLAGGPSSGEASHLS |
| within compound | | TAALGRLSAELHKLATLPRTETGSGSP |
| no. 88 | | |
| peptide backbone | 99 | HXHGTFTSDYSILLEEQAAREFIEWLLAGGPSSGEASHLS |
| within compound | | TAALGRLSAELHRLKTLPRTETGSGSP |
| no. 89 | | |
| peptide backbone | 100 | HXHGTFTSDYSILLEEQAAREFIEWLLAGGPSSGEASHLS |
| within compound | | TAALGRLSAELHRLATLPKTETGSGSP |
| no. 90 | | |
| peptide backbone | 101 | HXHGTFTSDYSILLEEQAAREFIEWLLAGGPSSGAGQAPG |
| within compound | | QAPGASHLSTAQTQRLSAELHRLKTLPRTETGSGSP |
| no. 91 | | |
| peptide backbone | 102 | HXHGTFTSDYSILLEEQAAREFIEWLLAGGPSSGAGQAPG |
| within compound | | QAPGASHLSTAQTQRLSAELHRLATLPKTETGSGSP |
| no. 92 | | |
| peptide backbone | 103 | HXHGTFTSDYSILLEEQAAREFIEWLLAGGPSSGAGQAPG |
| within compound | | QAPLASHLSTAQTQRLSAELHKLATLPRTETGSGSP |
| no. 93 | | |
| peptide backbone | 104 | YXEGTFTSDYSILLEEQAAREFIEWLLAGGPSRGEASHLS |
| within compound | | TAQTQRLSAELHKLATLPRTETGSGSP |
| no. 94 | | |
| peptide backbone | 105 | YXEGTFTSDYSILLEEQAAREFIEWLLAGGPSGGGGEASH |
| within compound | | LSTAQTQRLSAELHKLATLPRTETGSGSP |
| no. 95 | | |
| peptide backbone | 106 | YXEGTFTSDYSILLEEQAAREFIEWLLAGGPSGGGGEASH |
| within compound | | LSTAQTQRLSAELHKLATLPRTETGSGSP |
| no. 96 | | |
| peptide backbone | 107 | YXEGTFTSDYSILLEEQAAREFIEWLLAGGPSSGAGQAPG |
| within compound | | ASHLSTAQTQRLSAELHKLATLPRTETGSGSP |
| no. 97 | | |
| peptide backbone | 108 | YXEGTFTSDYSILLEEQAAREFIEWLLAGGPSSGAGQAPG |
| within compound | | QAPLASHLSTAQTARLSAELHKLATLPRTETGSGSP |
| no. 98 | | |
| peptide backbone | 109 | YXEGTFTSDYSILLEEQAAREFIEWLLAGGPSSGALQAPG |
| within compound | | QAPLASHLSTAQTQRLSAELHKLATLPRTETGSGSP |
| no. 99 | | |
| peptide backbone | 110 | HXHGTFTSDYSILLEEQAAREFIEWLLAGGPSKGAPPPSG |
| within compound | | GGEASHLSTAQTARLSAELHRLADLPRTETGSGSP |
| no. 100 | | |
| peptide backbone | 111 | HXHGTFTSDYSILLEEQAAREFIEWLLAGGPSKGAPPPSG |
| within compound | | GGEASHLSTAQTARLSAELHQLATLPRTETGSGSP |
| no. 101 | | |
| peptide backbone | 112 | YXEGTFTSDYSILLEEQAAREFIEWLLAGGPSSGAGQAPG |
| within compound | | QAPGASHLSTAQTQRLSAELHQLKTLPRTETGSGSP |
| no. 102 | | |
| peptide backbone | 113 | HXHGTFTSDYSILLEEQAAREFIEWLLAGGPSSGAGQAPG |
| within compound | | QAPGASHLSTAQTARLSAELHKLATLPRTETGSGSP |
| no. 103 | | |
| peptide backbone | 114 | HXHGTFTSDYSILLEEQAAREFIEWLLAGGPSSGAGQAPG |
| within compound | | QAPGASHLSTAQTARLSAELHKLATLPRTETGSGSP |
| no. 104 | | |
| peptide backbone | 115 | YXEGTFTSDYSILLEEQAAREFIEWLLAGGPSSGALQAPG |
| within compound | | QAPLASHLSTAQTARLSAELHKLATLPRTETGSGSP |
| no. 105 | | |
| peptide backbone | 116 | HXHGTFTSDYSILLEEQAAREFIEWLLAGGPSSGALQAPG |
| within compound | | QAPGASHLSTAQTARLSAELHRLKTLPRTETGSGSP |
| no. 106 | | |
| peptide backbone | 117 | HXHGTFTSDYSILLEEQAAREFIEWLLAGGPSSGALQAPG |
| within compound | | QAPGASHLSTAQTARLSAELHRLATLPKTETGSGSP |
| no. 107 | | |
| peptide backbone | 118 | HXHGTFTSDYSILLEEQAAREFIEWLLAGGPSSGALQAPG |
| within compound | | QAPLASHLSTAQTARLSAELHKLATLPRTETGSGSP |
| no. 108 | | |
| peptide backbone | 119 | YXEGTFTSDYSILLEEQAAREFIEWLLAGGPSSGAPPPSG |
| within compound | | GGEASHLSTAQTQRLSAKLHRLATLPRTETGSGSP |
| no. 109 | | |
| peptide backbone | 120 | YXEGTFTSDYSILLEEQAAREFIEWLLAGGPSSGAPPPSG |
| within compound | | GGEASHLSTAQTQRLSAKLHRLATLPRTETGSGSP |
| no. 110 | | |
| peptide backbone | 121 | YXEGTFTSDYSILLEEQAAREFIEWLLAGGPSSGAGQAPG |
| within compound | | QAPGASHLSTAQTQRLSAELHRLKTLPRTETGSGSP |
| no. 111 | | |
| peptide backbone | 122 | HXHGTFTSDYSILLEEQAAREFIEWLLAGGPSKGAPPPSG |
| within compound | | GGEASHLSTAQTARLSAELHRLADLPRTETGSGSP |
| no. 112 | | |
| peptide backbone | 123 | YXEGTFTSDYSILLEEQAAREFIEWLLAGGPSSGAGQAPG |
| within compound | | QAPGASHLSTAQTARLSAELHKLATLPRTETGSGSP |
| no. 113 | | |
| peptide backbone | 124 | YXEGTFTSDYSILLEEQAAREFIEWLLAGGPSSGAGQAPG |
| within compound | | QAPGASHLSTAALARLSAELHKLATLPRTETGSGSP |
| no. 114 | | |
| peptide backbone | 170 | YXEGTFTSDYSILLEEIAAREFIEWLLAGGPSSGAGQAPG |
| within compounds | | QAPGASHLSTAQTQRLSAELHKLATLPRTETGSGSP |
| 120, 160, 161, 192 | | |
| and 220 | | |
| peptide backbone | 171 | YXEGTFTSDYSILLEEIAAREFIEWLIAGGPSSGAGQAPGQ |
| within compound | | APGASHLSTAQTQRLSAELHKLATLPRTETGSGSP |
| no. 121 | | |
| peptide backbone | 172 | YXEGTFTSDYSKLLEEIAAREFIEWLLAGGPSSGAGQAPG |
| within compound | | QAPGASHLSTAQLGRLSAELHELATLPRTETGSGSP |
| no. 122 | | |
| peptide backbone | 173 | YXEGTFTSDYSKLLEEIAAREFIEWLLAGGPSSGAGQAPG |
| within compound | | QAPGASHLSTAQLGRLSAELHQLATLPRTETGSGSP |
| no. 123 | | |
| peptide backbone | 174 | YXEGTFTSDYSKLLEEIAAREFIEWLLAGGPSSGAGQAPG |
| within compound | | QAPGASHLSTAQTQRLSAELHELATLPRTETGSGSP |
| no. 124 | | |
| peptide backbone | 175 | YXEGTFTSDYSILLEEIAAREFIEWLLAGGPSSGAGQAPG |
| within compounds | | QAPLASHLSTAQTQRLSAELHKLATLPRTETGSGSP |
| 125, 142 and 221 | | |
| peptide backbone | 176 | YXEGTFTSDYSKLLEEIAAREFIEWLLAGGPSSGAGQAPG |
| within compound | | QAPGASHLSTAALGRLSAELHELATLPRTETGSGSP |
| no. 126 | | |
| peptide backbone | 177 | YXEGTFTSDYSKLLEEIAAREFIEWLIAGGPSSGAGQAPG |
| within compound | | QAPGASHLSTAALGRLSAELHELATLPRTETGSGSP |
| no. 127 | | |
| peptide backbone | 178 | YXEGTFTSDYSILLEEIAAREFIEWLLAGGPSSGAGQAPG |
| within compound | | QAPLASHLSTAQTARLSAELHKLATLPRTETGSGSP |
| no. 128 | | |
| peptide backbone | 179 | YXEGTFTSDYSKLLEEIAAREFIEWLLAGGPSSGAGQAPG |
| within compound | | QAPLASHLSTAQLGRLSAELHQLATLPRTETGSGSP |
| no. 129 | | |
| peptide backbone | 180 | YXEGTFTSDYSKLLEEIAAREFIEWLLAGGPSSGAGASHL |
| within compound | | STAQLGRLSAELHQLATLPRTETGSGSP |
| no. 130 | | |
| peptide backbone | 181 | YXEGTFTSDYSILLEEIAAREFIEWLLAGGPSSGAGASHLS |
| within compounds | | TAQTQRLSAELHKLATLPRTETGSGSP |
| 131 and 193 | | |
| peptide backbone | 182 | YXEGTFTSDYSILLEEIAAREFIEWLLAGGPSSGAGQAPG |
| within compound | | QAPLASQLSTAQTQRLSAELHKLATLPRTETGSGSP |
| no. 132 | | |
| peptide backbone | 183 | YXEGTFTSDYSILLEEIAAREFIEWLLAGGPSSGAGQAPG |
| within compound | | QAPLASELSTAQTQRLSAELHKLATLPRTETGSGSP |
| no. 133 | | |
| peptide backbone | 184 | YXEGTFTSDYSILLEEIAAREFIEWLLAGGPSSGEASHLST |
| within compound | | AQTQRLSAELHKLATLPRTETGSGSP |
| no. 134 | | |
| peptide backbone | 185 | YXEGTFTSDYSILLEEIAAREFIEWLLAGGPSSGAGQAPG |
| within compound | | QAPAASHLSTAQTQRLSAELHKLATLPRTETGSGSP |
| no. 135 | | |
| peptide backbone | 186 | YXEGTFTSDYSILLEEIAAREFIEWLLAGGPSSGAGQAPG |
| within compound | | QAPEASHLSTAQTQRLSAELHKLATLPRTETGSGSP |
| no. 136 | | |
| peptide backbone | 187 | YXEGTFTSDYSILLEEIAAREFIEWLLAGGPSSGAGQAPG |
| within compound | | QAPPASHLSTAQTQRLSAELHKLATLPRTETGSGSP |
| no. 137 | | |
| peptide backbone | 188 | YXEGTFTSDYSILLEEIAAREFIEWLLAGGPSSGAGQAPG |
| within compound | | QAPSASHLSTAQTQRLSAELHKLATLPRTETGSGSP |
| no. 138 | | |
| peptide backbone | 189 | YXEGTFTSDYSILLEEIAAREFIEWLLAGGPSSGAGQAPG |
| within compound | | QAPVASHLSTAQTQRLSAELHKLATLPRTETGSGSP |
| no. 139 | | |
| peptide backbone | 190 | YXEGTFTSDYSILLEEIAAREFIEWLLAGGPSSGAGGGGA |
| within compound | | SHLSTAQTQRLSAELHKLATLPRTETGSGSP |
| no. 140 | | |
| peptide backbone | 191 | YXEGTFTSDYSILLEEIAAREFIEWLLAGGPSSGAGGGGA |
| within compound | | GELSTAQTQRLSAELHKLATLPRTETGSGSP |
| no. 141 | | |
| peptide backbone | 192 | YXEGTFTSDYSILLEEIAAREFIEWLIAGGPSSGAGQAPGQ |
| within compound | | APLASHLSTAQTQRLSAELHKLATLPRTETGSGSP |
| no. 143 | | |
| peptide backbone | 193 | YXEGTFTSDYSILLEEIAAREFIEWLLAGGPSSGAGQAPG |
| within compound | | QAPGASELSTAQTQRLSAELHKLATLPRTETGSGSP |
| no. 144 | | |
| peptide backbone | 194 | YXEGTFTSDYSILLEEIAAREFIEWLLAGGPSSGAGQAPG |
| within compound | | QAPGASELSTAQTARLSAELHKLATLPRTETGSGSP |
| no. 145 | | |
| peptide backbone | 195 | YXEGTFTSDYSILLEEIAAREFIEWLLAGGPSSEAGQAPGQ |
| within compounds | | APGASHLSTAQTQRLSAELHKLATLPRTETGSGSP |
| 146 and 211 | | |
| peptide backbone | 196 | YXEGTFTSDYSILLEEIAAREFIEWLLAGGPSSGEGQAPG |
| within compounds | | QAPGASHLSTAQTQRLSAELHKLATLPRTETGSGSP |
| 147 and 212 | | |
| peptide backbone | 197 | YXEGTFTSDYSILLEEIAAREFIEWLLAGGPSSGAGEAPGQ |
| within compounds | | APGASHLSTAQTQRLSAELHKLATLPRTETGSGSP |
| 148 and 213 | | |
| peptide backbone | 198 | YXEGTFTSDYSILLEEIAAREFIEWLLAGGPSSGAGQEPG |
| within compounds | | QAPGASHLSTAQTQRLSAELHKLATLPRTETGSGSP |
| 149 and 214 | | |
| peptide backbone | 199 | YXEGTFTSDYSILLEEIAAREFIEWLLAGGPSSGAGQAEG |
| within compounds | | QAPGASHLSTAQTQRLSAELHKLATLPRTETGSGSP |
| 150 and 215 | | |
| peptide backbone | 200 | YXEGTFTSDYSILLEEIAAREFIEWLLAGGPSSGAGQAPEQ |
| within compounds | | APGASHLSTAQTQRLSAELHKLATLPRTETGSGSP |
| 151 and 216 | | |
| peptide backbone | 201 | YXEGTFTSDYSILLEEIAAREFIEWLLAGGPSSGAGQAPGE |
| within compounds | | APGASHLSTAQTQRLSAELHKLATLPRTETGSGSP |
| 152 and 217 | | |
| peptide backbone | 202 | YXEGTFTSDYSILLEEIAAREFIEWLLAGGPSSGAGQAPG |
| within compounds | | QEPGASHLSTAQTQRLSAELHKLATLPRTETGSGSP |
| 153 and 218 | | |
| peptide backbone | 203 | YXEGTFTSDYSILLEEIAAREFIEWLLAGGPSSGAGQAPG |
| within compounds | | QAEGASHLSTAQTQRLSAELHKLATLPRTETGSGSP |
| 154 and 219 | | |
| peptide backbone | 204 | YXEGTFTSDYSILLEEIAAREFIEWLIAGGPSSGAGQEPGQ |
| within compound | | EPGASHLSTAQTQRLSAELHKLATLPRTETGSGSP |
| no. 155 | | |
| peptide backbone | 205 | YXEGTFTSDYSILLEEIAAREFIAWLIAGGPSSGAGQEPGQ |
| within compound | | EPGASHLSTAQTQRLSAELHKLATLPRTETGSGSP |
| no. 156 | | |
| peptide backbone | 206 | YXEGTFTSDYSILLEEIAAREFIQWLIAGGPSSGAGQEPGQ |
| within compound | | EPGASHLSTAQTQRLSAELHKLATLPRTETGSGSP |
| no. 157 | | |
| peptide backbone | 207 | YXEGTFTSDYSILLEEIAAREFIEWLIAGGPSSGAGASHLS |
| within compound | | TAQTQRLSAELHKLATLPRTETGSGSP |
| no. 158 | | |
| peptide backbone | 208 | YXEGTFTSDYSILLEEIAAREFIEWLLAGGPSSGAGQAPG |
| within compound | | QAPASHLSTAQTQRLSAELHKLATLPRTETGSGSP |
| no. 159 | | |
| peptide backbone | 209 | YXEGTFTSDYSILLEEIAAREFIEWLLAGGPSSGAGQAPG |
| within compound | | QAPGASHLSTAQTQRLSAELHKLATEPRTETGSGSP |
| no. 162 | | |
| peptide backbone | 210 | YXEGTFTSDYSILLEEIAAREFIEWLLAGGPSSGAGQAPG |
| within compound | | QAPGASELSTAQTQRLSAELHKLATEPRTETGSGSP |
| no. 163 | | |
| peptide backbone | 211 | YXEGTFTSDYSILLEEIAAREFIEWLLAGGPSSGAGQAPGE |
| within compound | | APGASHLSTAQTQRLSAELHKLATEPRTETGSGSP |
| no. 164 | | |
| peptide backbone | 212 | YXEGTFTSDYSILLEEIAAREFIEWLLAGGPSSEAGQAPGQ |
| within compound | | APGASHLSTAQTQRLSAELHKLATEPRTETGSGSP |
| no. 165 | | |
| peptide backbone | 213 | YXEGTFTSDYSILLEEIAAREFIEWLLAGGPSSGAGQAPGE |
| within compound | | APGASELSTAQTQRLSAELHKLATLPRTETGSGSP |
| no. 166 | | |
| peptide backbone | 214 | YXEGTFTSDYSILLEEIAAREFIEWLLAGGPSSEAGQAPGQ |
| within compound | | APGASELSTAQTQRLSAELHKLATLPRTETGSGSP |
| no. 167 | | |
| peptide backbone | 215 | YXEGTFTSDYSILLEEIAAREFIEWLLAGGPSSEAGQAPGE |
| within compound | | APGASHLSTAQTQRLSAELHKLATLPRTETGSGSP |
| no. 168 | | |
| peptide backbone | 216 | YXEGTFTSDYSKLLEEIAAREFIEWLLAGGPSSGAGQAPG |
| within compound | | QAPGASELSTAALGRLSAELHELATLPRTETGSGSP |
| no. 169 | | |
| peptide backbone | 217 | YXEGTFTSDYSKLLEEIAAREFIEWLLAGGPSSGAGQAPG |
| within compound | | QAPGASELSTAALGRLSAELHQLATLPRTETGSGSP |
| no. 170 | | |
| peptide backbone | 218 | YXEGTFTSDYSKLLEEIAAREFIEWLLAGGPSSGAGQAPG |
| within compound | | EAPGASELSTAALGRLSAELHQLATLPRTETGSGSP |
| no. 171 | | |
| peptide backbone | 219 | YXEGTFTSDYSKLLEEIAAREFIEWLLAGGPSSGAGQAPG |
| within compound | | EAPGASELSTAALGRLSAELHQLATEPRTETGSGSP |
| no. 172 | | |
| peptide backbone | 220 | YXEGTFTSDYSKLLEEIAAREFIEWLLAGGPSSGAGQAPG |
| within compound | | EAPGASELSTAQLGRLSAELHQLATEPRTETGSGSP |
| no. 173 | | |
| peptide backbone | 221 | YXEGTFTSDYSKLLEEIAAREFIEWLLAGGPSSGAGQAPG |
| within compound | | EAPGASELSTAQTGRLSAELHQLATEPRTETGSGSP |
| no. 174 | | |
| peptide backbone | 222 | YXEGTFTSDYSKLLEEIAAREFIEWLLAGGPSSGAGQAPG |
| within compound | | EAPGASELSTAQTQRLSAELHQLATEPRTETGSGSP |
| no. 175 | | |
| peptide backbone | 223 | YXEGTFTSDYSILLEEIAAREFIEWLLAGGPSSGAGASELS |
| within compound | | TAQTQRLSAELHKLATLPRTETGSGSP |
| no. 176 | | |
| peptide backbone | 224 | YXEGTFTSDYSILLEEIAAREFIEWLLAGGPSSEAGASHLS |
| within compound | | TAQTQRLSAELHKLATLPRTETGSGSP |
| no. 177 | | |
| peptide backbone | 225 | YXEGTFTSDYSILLEEIAAREFIEWLLAGGPSSEAGASELS |
| within compound | | TAQTQRLSAELHKLATLPRTETGSGSP |
| no. 178 | | |
| peptide backbone | 226 | YXEGTFTSDYSILLEEIAAREFIEWLLAGGPSSGAGASHLS |
| within compound | | TAQTQRLSAELHKLATEPRTETGSGSP |
| no. 179 | | |
| peptide backbone | 227 | YXEGTFTSDYSILLEEIAAREFIEWLLAGGPSSGAGASQLS |
| within compound | | TAQTQRLSAELHKLATEPRTETGSGSP |
| no. 180 | | |
| peptide backbone | 228 | YXEGTFTSDYSILLEEIAAREFIEWLLAGGPSSEAGASQLS |
| within compound | | TAQTQRLSAELHKLATLPRTETGSGSP |
| no. 181 | | |
| peptide backbone | 229 | YXEGTFTSDYSILLEEIAAREFIEWLLAGGPSSEAGASQLS |
| within compound | | TAQTQRLSAELHKLATEPRTETGSGSP |
| no. 182 | | |
| peptide backbone | 230 | YXEGTFTSDYSKLLEEIAAREFIEWLLAGGPSSGAGASEL |
| within compound | | STAQLGRLSAELHQLATLPRTETGSGSP |
| no. 183 | | |
| peptide backbone | 231 | YXEGTFTSDYSKLLEEIAAREFIEWLLAGGPSSGAGASHL |
| within compound | | STAQLGRLSAELHQLATEPRTETGSGSP |
| no. 184 | | |
| peptide backbone | 232 | YXEGTFTSDYSKLLEEIAAREFIEWLLAGGPSSEAGASHLS |
| within compound | | TAQLGRLSAELHQLATEPRTETGSGSP |
| no. 185 | | |
| peptide backbone | 233 | YXEGTFTSDYSKLLEEIAAREFIEWLLAGGPSSGAGASEL |
| within compound | | STAQLGRLSAELHQLATEPRTETGSGSP |
| no. 186 | | |
| peptide backbone | 234 | YXEGTFTSDYSKLLEEIAAREFIEWLLAGGPSSEAGASELS |
| within compound | | TAQLGRLSAELHQLATLPRTETGSGSP |
| no. 187 | | |
| peptide backbone | 235 | YXEGTFTSDYSKLLEEIAAREFIEWLLAGGPSSEAGASELS |
| within compound | | TAQLGRLSAELHQLATEPRTETGSGSP |
| no. 188 | | |
| peptide backbone | 236 | YXEGTFTSDYSILLEEIAAREFIEWLLAGGPSSGAGEAPGE |
| within compound | | APGASHLSTAQTQRLSAELHKLATLPRTETGSGSP |
| no. 189 | | |
| peptide backbone | 237 | YXEGTFTSDYSILLEEIAAREFIEWLIAGGPSSGAGEAPGQ |
| within compound | | APGASQLSTAQTQRLSAELHKLATLPRTETGSGSP |
| no. 190 | | |
| peptide backbone | 238 | YXEGTFTSDYSILLEEIAAREFIEWLIAGGPSSGAGEAPGQ |
| within compound | | APGASELSTAQTQRLSAELHKLATLPRTETGSGSP |
| no. 191 | | |
| peptide backbone | 239 | YXEGTFTSDYSILLEEIAAQEFIEWLLQGGPSSGAGASELS |
| within compound | | TAQTQRLSAELHKLATLPRTETGSGSP |
| no. 194 | | |
| peptide backbone | 240 | YXEGTFTSDYSILLEEIAAQEFIEWLLQGGPSSGAGEAPG |
| within compound | | QAPGASELSTAQTQRLSAELHKLATLPRTETGSGSP |
| no. 195 | | |
| peptide backbone | 241 | YXEGTFTSDYSILLEEIAAREFIEWLLAGGPSSGAGQAPGE |
| within compound | | APLASHLSTAQTQRLSAELHKLATLPRTETGSGSP |
| no. 196 | | |
| peptide backbone | 242 | YXEGTFTSDYSILLEEIAAREFIEWLLAGGPSSGAGQAPG |
| within compound | | QAPLASHLSTAQTQRLSAELHKLATEPRTETGSGSP |
| no. 197 | | |

wherein X represents always Aib.

### DESCRIPTION

The present invention relates to a GLP-1-/GIP-/amylin-receptor tri-agonist comprising a peptide according to Formula I:

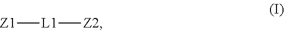

comprising one lysine (Lys, K) residue; wherein:
Z1 is a peptide comprising a maximum of 4 amino acid substitutions relative to Formula II (SEQ ID NO: 1):

(II)
YX₂EGTFTSDYSILLEEQAAREFIEWLLAGGPSKG,

wherein the amino acid at position X2 represents Aib;
L1 is a peptide linker; and
Z2 is a peptide comprising a C-terminal amide and a maximum of 10 amino acid substitutions relative to Formula III (SEQ ID NO: 2):

(III)
ASELSTAALGRLSAELHELATLPRTETGSGSP.

In another aspect the invention relates to a GLP-1-/GIP-/amylin-receptor tri-agonist comprising a peptide according to Formula I:

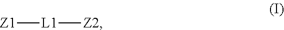

comprising one lysine (Lys, K) residue; wherein:
Z1 is a peptide having a maximum of 4 amino acid substitutions relative to Formula II (SEQ ID NO: 1):

(II)
YX₂EGTFTSDYSILLEEQAAREFIEWLLAGGPSKG,

wherein the amino acid at position X₂ represents Aib,
and wherein
Z1 comprises an amino acid sequence according to Formula X (SEQ ID NO: 161):

(X)
X₂₁X₂₂X₂₃GTFTSDYSX₂₄LLEEX₂₅AAX₂₆EFIX₂₇WLX₂₈X₂₉GGPSX₃₀X₃₁,

wherein
X₂₁ represents His (H) or Tyr (Y),
X₂₂ represents Aib,
X₂₃ represents Glu (E) or His (H),
X₂₄ represents Ile (I) or Lys (K),
X₂₅ represents Gln (Q) or Ile (I),
X₂₆ represents Arg (R) or Gln (Q),
X₂₇ represents Ala (A), Glu (E) or Gln (Q),
X₂₈ represents Leu (L) or I (Ile),
X₂₉ represents Ala (A) or Gln (Q),
X₃₀ represents Arg (R), Gly (G), Lys (K) or Ser (S),
X₃₁ represents Gly (G), Glu (E) or Lys (K);
   L1 is a peptide linker; and
   Z2 is a peptide comprising a C-terminal amide, and having a maximum of 10 amino acid substitutions relative to Formula III (SEQ ID NO: 2):

(III)
ASELSTAALGRLSAELHELATLPRTETGSGSP,

and
Z2 comprises an amino acid sequence according to Formula XII (SEQ ID NO:164):

(XII)
AX₃₂X₃₃LSTAX₃₄X₃₅X₃₆RLSAX₃₇LHX₃₈LX₃₉X₄₀X₄₁PX₄₂TETGSGX₄₃P,

wherein
X₃₂ represents Gly (G) or Ser (S),
X₃₃ represents Gln (Q), Glu (E), His (H) or Lys (K),
X₃₄ represents Ala (A) or Gln (Q),
X₃₅ represents Gln (Q), Leu (L) or Thr (T),
X₃₆ represents Ala (A), Gly (G) or Gln (Q),
X₃₇ represents Glu (E) or Lys (K),
X₃₈ represents Arg (R), Gln (Q), Glu (E), Gly (G), His (H), Lys (K), Thr (T) or Tyr (Y),
X₃₉ represents Ala (A) or Lys (K),
X₄₀ represents Asp (D) or Thr (T),
X₄₁ represents Leu (L) or Glu (E),
X₄₂ represents Arg (R) or Lys (K),
X₄₃ represents Ala (A) or Ser (S).

In another aspect the invention relates to a GLP-1-/GIP-/amylin-receptor tri-agonist comprising a peptide according to Formula I:

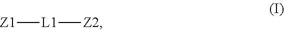

comprising on lysine (Lys, K) residue; wherein:
Z1 is a peptide having a maximum of 4 amino acid substitutions relative to Formula II (SEQ ID NO: 1):

(II)
YX₂EGTFTSDYSILLEEQAAREFIEWLLAGGPSKG,

wherein the amino acid at position X₂ represents Aib, and wherein
Z1 comprises an amino acid sequence according to Formula IIa (SEQ ID NO: 3):

(IIa)
X₁X₂X₃GTFTSDYSILLEEQAAREFIEWLLAGGPSX₄X₅,

wherein
X₁ represents His (H) or Tyr (Y),
X₂ represents Aib,
X₃ represents Glu (E) or His (H),
X₄ represents Arg (R), Gly (G), Lys (K) or Ser (S),
X₅ represents Gly (G) or Lys (K);
   L1 is a peptide linker; and
   Z2 is a peptide comprising a C-terminal amide and having a maximum of 10 amino acid substitutions relative to Formula III (SEQ ID NO: 2):

(III)
ASELSTAALGRLSAELHELATLPRTETGSGSP,

and
Z2 comprises an amino acid sequence according to Formula IIIa (SEQ ID NO: 4):

(IIIa)
ASX₆LSTAX₇X₈X₉RLSAX₁₀LHX₁₁LX₁₂X₁₃LPX₁₄TETGSGX₁₅P,

wherein
X₆ represents Gln (Q), Glu (E), His (H) or Lys (K),
X₇ represents Ala (A) or Gln (Q),
X₈ represents Gln (Q), Leu (L) or Thr (T),
X₉ represents Ala (A), Gly (G) or Gln (Q),
X₁₀ represents Glu (E) or Lys (K),
X₁₁ represents Arg (R), Gln (Q), Glu (E), Gly (G), His (H), Lys (K), Thr (T) or Tyr (Y),
X₁₂ represents Ala (A) or Lys (K),
X₁₃ represents Asp (D) or Thr (T),
X₁₄ represents Arg (R) or Lys (K),
X₁₅ represents Ala (A) or Ser (S).

In another aspect the invention relates to a GLP-1-/GIP-/amylin-receptor tri-agonist comprising a peptide according to Formula I:

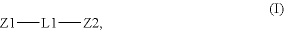

comprising one lysine (Lys, K) residue; wherein:
Z1 is a peptide having a maximum of 4 amino acid substitutions relative to Formula II (SEQ ID NO: 1):

(II)
YX₂EGTFTSDYSILLEEQAAREFIEWLLAGGPSKG,

wherein the amino acid at position X₂ represents Aib,
and wherein
Z1 comprises an amino acid sequence according to Formula Xa (SEQ ID NO: 162):

(Xa)
YX₅₁EGTFTSDYSX₅₂LLEEIAAX₅₃EFIX₅₄WLX₅₅X₅₆GGPSSX₅₇,

wherein
X₅₁ represents Aib,
X₅₂ represents Ile (I) or Lys (K),
X₅₃ represents Arg (R) or Gln (Q),
X₅₄ represents Ala (A), Glu (E) or Gln (Q),
X₅₅ represents Leu (L) or I (Ile),
X₅₆ represents Ala (A) or Gln (Q),
X₅₇ represents Gly (G) or Glu (E);
   L1 is a peptide linker; and
   Z2 is a peptide comprising a C-terminal amide, and having a maximum of 10 amino acid substitutions relative to Formula III (SEQ ID NO: 2):

(III)
ASELSTAALGRLSAELHELATLPRTETGSGSP,

and
Z2 comprises an amino acid sequence according to Formula XIIa (SEQ ID NO: 165):

(XIIa)
AX₅₈X₅₉LSTAX₆₀X₆₁X₆₂RLSAELHX₆₃LATX₆₄PRTETGSGSP,

wherein
X₅₈ represents Gly (G) or Ser (S),
X₅₉ represents Gln (Q), Glu (E), or His (H),
X₆₀ represents Ala (A) or Gln (Q),
X₆₁ represents Leu (L) or Thr (T),
X₆₂ represents Ala (A), Gly (G) or Gln (Q),
X₆₃ represents Gln (Q), Glu (E), or Lys (K),
X₆₄ represents Leu (L) or Glu (E).

The compounds disclosed herein are agonists at each of the receptors GLP-1, GIP, and amylin. Hence the compound of the present invention is a GLP-1 receptor agonist and a GIP receptor agonist and an agonist for the amylin receptor (i.e. an amylin receptor agonist). It is capable of activating or “agonising” all three of the GLP-1 receptor, the GIP receptor, and the amylin receptor: it is a “GLP-1-/GIP-/amylin-receptor tri-agonist”. The GLP-1-/GIP-/amylin-receptor tri-agonist may provide a similar level of activation of all three GLP-1, GIP, and amylin receptors; then it is referred to as a “balanced GLP-1-/GIP-/amylin-receptor tri-agonist” or in short a “balanced tri-agonist”.

A “receptor agonist” or “agonist” may be defined as a ligand, such as a compound, that binds to and activates a biological receptor to produce a biological response. A full agonist may be defined as one that elicits a response of the same magnitude as the natural ligand (see e.g., “Principles of Biochemistry”, A L Lehninger, D L Nelson, M M Cox, Second Edition, Worth Publishers, 1993, page 763). Receptors can be activated by either endogenous agonists, such as endogenous hormones, or exogenous agonists, such as pharmaceutical drugs.

In the context of the current invention, a “co-agonist” is a compound capable of binding to and activating two different biological receptors, e.g. a compound comprising two different ligands, each of which binds to a given biological receptor, to produce a biological response that is characteristic of the natural ligands. In an analogous way, a “triple agonist” or “tri-agonist” is a compound capable of binding to and activating three different biological receptors, e.g. a compound comprising three different ligands, each of which binds to a given biological receptor, to produce a biological response that is characteristic of the natural ligands.

A “GLP-1 receptor agonist” may be defined as a compound which is capable of binding to the GLP-1 receptor and capable of activating it. A “full” GLP-1 receptor agonist may be defined as a GLP-1 receptor agonist which is capable of eliciting a magnitude of GLP-1 receptor response that is similar to native glucagon like peptide 1 (GLP-1). Sema-glutide, disclosed in WO 2006/097537, Example 4, is an example of an exogenous GLP-1 receptor agonist.

A “GIP receptor agonist” may be defined as a compound which is capable of binding to the GIP receptor and capable of activating it. A “full” GIP receptor agonist may be defined as a GIP receptor agonist which is capable of eliciting a magnitude of GIP receptor response that is similar to native glucose-dependent insulinotropic polypeptide (GIP).

A “GLP-1/GIP receptor co-agonist” may be defined as a compound which is capable of binding to both, the GLP-1 receptor, and the GIP receptor and capable of activating both receptors. An example of a GLP-1/GIP co-agonist is tirzepatide, which is described in WO 2016/111971.

An “amylin receptor agonist” may be defined as a compound which is capable of binding to the amylin receptors (AMYRs) and the calcitonin receptor (CTR) and capable of activating it. Amylin receptors consist of heterodimers of two components: the calcitonin receptor (CTR) and one of three receptor activity-modifying proteins (RAMP1-3), resulting in three possible complexes, AMYR1-3. Unless otherwise specified herein, “amylin receptor” at least refers to amylin receptor 3 (AMYR3). Nonetheless, some concomitant activity on the other receptors can be expected. A “full” amylin receptor agonist may be defined as an amylin receptor agonist which is capable of eliciting a magnitude of amylin receptor response that is similar to native amylin. An amylin receptor agonist will often also be a calcitonin receptor agonist. Examples of amylin receptor agonists are human amylin, human calcitonin and cagrilintide (disclosed in WO 2012/168432). It is noted that all headings and sub-headings are used herein for convenience only and should not be construed as limiting the invention in any way.

The use of any and all examples, or exemplary language (e.g., “such as”) provided herein, is intended merely to better illuminate the invention, and does not pose a limitation on the scope of the invention unless otherwise claimed.

In order that the present invention may be more readily understood, certain terms are first defined.

In what follows, Greek letters may be represented by their symbol or the corresponding written name, for example: α=alpha; β=beta; γ=gamma; ε=epsilon; ω=omega; etc. Also, the Greek letter of μ may be represented by “u”, e.g. in μl=ul, or in μM=uM.

Unless otherwise indicated in the specification, terms presented in singular form generally also include the plural situation. The term “a” or “an” is intended to mean “one or more.”

The term “comprise” and variations thereof such as “comprises” and “comprising,” when preceding the recitation of a step or an element, are intended to mean that the addition of further steps or elements is optional and not excluded. As disclosed herein the open-ended terms like “comprises” and “comprising” might be replaced with closed terms such as “consists of”, “consisting of”, and the like.

The term “about” is used herein to mean approximately, roughly, or around. When the term “about” is used in conjunction with a numerical range, it modifies that range by extending the boundaries above and below the numerical values set forth. In general, the term “about” can modify a numerical value above and below the stated value by 10 percent, up or down (higher or lower).

Amino acids are molecules containing an amine group and a carboxylic acid group, and, optionally, one or more additional groups, often referred to as a side chain.

The term “amino acid” includes canonical amino acids (which are genetically encoded), and unnatural amino acids. Non-limiting examples of unnatural amino acids are Aib (α-aminoisobutyric acid or 2-aminoisobutyric acid), deamino histidine (alternative name 3-(imidazol-4-yl)propanoic acid, abbreviated Imp (imidazopropionyl)) and the d-isomers of the canonical amino acids. All amino acid residues within the peptide for which the optical isomer is not stated is herein to be understood to mean the L-isomer, unless otherwise specified.

Herein, “amino acid substitution” or “substitution” refers to one or more amino acid(s) being replaced with the same number of amino acid(s) in the backbone of the peptide. Substitutions may be, but are not limited to, conservative substitutions. For example, an amino acid may be substituted to an amino acid with similar biochemical properties, for example, a basic amino acid may be substituted to another basic amino acid (e.g. lysine to arginine), an acidic amino acid may be substituted to another acidic amino acid (e.g. glutamate to aspartate), a neutral amino acid may be substituted to another neutral amino acid (e.g. threonine to serine), a charged amino acid may be substituted to another charged amino acid (e.g. glutamate to lysine), a hydrophilic amino acid may be substituted to another hydrophilic amino acid (e.g. asparagine to glutamine), a hydrophobic amino acid may be substituted to another hydrophobic amino acid (e.g. alanine to valine), a polar amino acid may be substituted to another polar amino acid (e.g. serine to threonine), an aromatic amino acid may be substituted to another aromatic amino acid (e.g. phenylalanine to tryptophan) and an aliphatic amino acid may be substituted to another aliphatic amino acid (e.g. leucine to isoleucine).

The term “excipient” as used herein broadly refers to any component other than the Active Pharmaceutical Ingredient (API).

The term “identity” or “sequence identity” as known in the art, refers to a relationship between the sequences of two or more polypeptides, as determined by comparing the sequences. In the art, “identity” also means the degree of sequence relatedness between polypeptides, as determined by the number of matches between strings of two or more amino acid residues. “Identity” measures the percentage of identical matches between the smaller of two or more sequences with gap alignments (if any) addressed by a particular mathematical model or computer program (i.e., “algorithms”). Identity of related polypeptides can be readily calculated by known methods, e.g. using Needleman (Needleman et al. J. Mol. Biol. 1970; 48:443-453) from EMBOSS-6.6.0 using the parameters 10 and 0.5 for gaps opening and extensions, respectively (gapopen=10, gapextend=0.5) or e.g. calculated by: (I) comparing two optimally aligned sequences over a window of comparison (e.g., the length of the longer sequence, the length of the shorter sequence, a specified window, etc.), (2) determining the number of positions containing identical monomers (e.g., same amino acids occurs in both sequences) to yield the number of matched positions, (3) dividing the number of matched positions by the total number of positions in the comparison window (e.g., the length of the longer sequence, the length of the shorter sequence, a specified window), and (4) multiplying the result by 100 to yield the percent “sequence identity”. For example, if peptides A and B are both 20 amino acids in length and have identical amino acids at all but 1 position, then peptide A and peptide B have 95% sequence identity.

The term “polypeptide” or “peptide” as used herein includes oligopeptides and refers to a single chain of amino acids connected by one or more amide (or peptide) bonds. The terms “polypeptide” and “peptide” shall be used interchangeably herein.

The term “half-life” or “plasma half-life” as used herein refers to the time required for half the quantity of a substance administered to a person to be metabolized or eliminated from the serum or plasma of the person by normal biological processes.

The term “treatment” and variations thereof, as used herein, refers to the medical therapy of any human subject in need thereof. The term includes administering a therapeutically effective amount of a peptide as disclosed herein sufficient to reduce or eliminate at least one symptom of the disorder in question. “Treatment”, however, need not be a cure. The timing and purpose of said treatment may vary from one individual to another, according to the status quo of the subject's health. Thus, said treatment may be prophylactic, palliative, symptomatic and/or curative. In terms of the present invention, prophylactic, palliative, symptomatic and/or curative treatments may represent separate aspects of the invention.

As used herein the terms “preventing”, “prevent” or “prevention” or variations thereof refers to protecting a subject from developing at least one symptom of a disease or reducing the severity of a symptom of a disorder.

### GLP-1-/GIP-/Amylin-Receptor Tri-Agonist

The compounds disclosed herein are herein referred to as “GLP-1-/GIP-/amylin-receptor tri-agonist” or “GLP-1 receptor-GIP receptor-amylin receptor tri-agonists” or “GLP-1-/GIP-/amylin-receptor triple agonist” or “GLP-1 receptor-GIP receptor-amylin receptor triple agonists”.

The GLP-1-/GIP-/amylin-receptor tri-agonist comprises a peptide Z1, which is a GLP-1-/GIP receptor co-agonist, a peptide linker L1, and a peptide Z2, which is an amylin receptor agonist. The GLP-1-/GIP-/amylin-receptor tri-agonist is a compound that binds to each of the three GLP-1, GIP, and amylin receptors and is capable of activating each receptor GLP-1R, GIPR, and amylin receptor, thus eliciting a response at each receptor.

The peptide Z1 disclosed herein may have a maximum of 4 amino acid substitutions, relative to Formula II (SEQ ID NO: 1). The peptide Z2 disclosed herein may have a maximum of 10 amino acid substitution, relative to Formula III (SEQ ID NO: 2).

The term “compound” is used herein to refer to a molecular entity, and “compounds” may thus have different structural elements besides the minimum element defined for each compound or group of compounds. It follows that a compound may be a peptide or a derivative thereof, as long as the compound comprises the defined structural and/or functional elements. The term “compound” is also meant to cover pharmaceutically relevant forms thereof, i.e. a compound as defined herein or a pharmaceutically acceptable salt, amide, or ester thereof.

The compound disclosed herein may be a potent GLP-1 receptor agonist.

The compound disclosed herein may be a potent GIP receptor agonist.

The compound disclosed herein may be a potent amylin receptor agonist.

The in vitro potency of the agonists may be measured as described in the assays of Example 4. The term “potency” is used to describe the effect of a given compound in assays where a sigmoidal relationship between log concentration and the effect of a compound has been established. Furthermore, the response should be variable from 0 to 100%. The potency of the compound may be described by means of its EC(effective concentration)₅₀ values. EC₅₀ represents the concentration of compound upon which 50% of its maximal effect is observed in the assay, e.g., as described in Example 4. The lower the EC₅₀ value, the more potent the compound.

The compound disclosed herein may provide a similar level of activation of all three GLP-1, GIP, and amylin receptors; that is, it may be “balanced” and is referred to as a “balanced GLP-1-/GIP-/amylin-receptor tri-agonist” or in short a “balanced tri-agonist”. Relatively “balanced” receptor activation is advantageous because the relative ratio of the compound's GLP-1, GIP, and amylin receptor agonist activities is locked to the molecule; it is not possible to titrate the three receptor agonists, relative to one another. Ultimately, where a molecule is “balanced”, it may be dosed such that all three hormone systems are activated without side-effects outweighing benefits.

A tri-agonist whose potency ratio (A/B) of the potency (A) of the receptor with lowest potency (i.e. the highest numerical EC₅₀ value) divided by the potency (B) of the receptor with highest potency (i.e. the lowest numerical EC₅₀ value) is less than 50 is defined as a “balanced tri-agonist” or “balanced GLP-1-/GIP-/amylin-receptor tri-agonist” (based on the assays in the absence of human serum albumin (HSA), as shown in Example 4 Table 7a and Table 8a). For example, compound 10 has an EC₅₀ of 13.83 pM on the GLP-1 receptor, an EC₅₀ of 2.11 pM on the GIP receptor, and an EC₅₀ of 6.8 pM on the amylin receptor. Hence it has lowest potency on the hGLP-1 and (A) corresponds to 13.83 pM, and it has its highest potency on the GIP receptor with 2.11 pM, which corresponds to (B). Hence the potency ratio (A/B) is 13.83 pM (A) divided by 2.11 pM (B) is equal to 7 (rounded accordingly), this means that compound 10 is a “balanced tri-agonist”.

A compound that is potent on one receptor and much less potent on the other(s) would be “unbalanced”. Such an “unbalanced tri-agonist” is defined as compound having a potency ratio (A/B) of 50 or more. For example, reference compound 1 has a potency ratio (A/B) of 287 (i.e., 1516.05 pM (=A) divided by 5.29 pM (=B), rounded accordingly). For example, this reference compound being potent (i.e. EC₅₀ value of <30 pM) on the GIP and amylin receptors and less potent (i.e. EC₅₀ value of 1516 pM) on the GLP-1 receptor, would be unable to achieve optimal efficacy from all three hormone systems because side-effects arising from activation of the GIP and amylin receptors would prevent administration of a dose sufficiently high to achieve activation of the GLP-1 hormone system as well. The opposite situation might occur if the compound were potent on one receptor, e.g. the GLP-1 receptor (for example, having an EC₅₀ value of <50 pM) and considerably less potent on the two other receptors, e.g. GIP and amylin receptors (for example, having an EC₅₀ value of >500 pM each).

### Peptide

The present invention relates in one aspect to a GLP-1-/GIP-/amylin-receptor tri-agonist comprising a peptide according to Formula I:

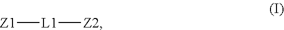

comprising one lysine (Lys, K) residue; wherein:
Z1 is a peptide comprising a maximum of 4 amino acid substitutions relative to Formula II (SEQ ID NO: 1):

(II)
YX₂EGTFTSDYSILLEEQAAREFIEWLLAGGPSKG,

wherein the amino acid at position X2 represents Aib;
L1 is a peptide linker; and
Z2 is a peptide comprising a C-terminal amide and a maximum of 10 amino acid substitutions relative to Formula III (SEQ ID NO: 2):

(III)
ASELSTAALGRLSAELHELATLPRTETGSGSP.

The GLP-1-/GIP-/amylin-receptor tri-agonist comprises a peptide, Z1-L1-Z2 comprising a peptide Z1, a peptide linker L1, and a peptide Z2.

The peptide Z1 is a GLP-1-/GIP receptor co-agonist which is capable of binding to both, the GLP-1 receptor, and the GIP receptor and capable of activating both receptors. The C-terminus of peptide Z1 is attached to the peptide linker L1 via a peptide bond.

L1 is a peptide linker. Its N-terminus is attached to the C-terminus of Z1 and its C-terminus is attached to the N-terminus of Z2 via a peptide bond.

The peptide Z2 is an amylin receptor agonist which is capable of binding to at least amylin receptor and capable of activating it. The N-terminus of Z2 is attached to the C-terminus of L1 via a peptide bond. The C-terminus of Z2 is modified with an amide group, which is considered essential for bioactivity. In a preferred embodiment, the amine group of the C-terminal amide is NH₂.

The molecular format may be a single chain peptide backbone comprising one lysine (Lys, K) residue. The one lysine (Lys, K) residue may be present in the peptide Z1 portion of the peptide backbone, or the one lysine (Lys, K) residue may be present in the peptide Z2 portion of the peptide backbone. The one lysine residue may be covalently bound to a protraction moiety, which may be referred to herein as “L_{P}-P”, wherein “L_{P}” is an optional linker and “P” is a protractor. The peptide backbone of the GLP-1-/GIP-/amylin-receptor tri-agonist of the present invention typically comprises about 66 to about 80 amino acid residues linked together by peptide bonds.

The peptide Z1 disclosed herein may have a maximum of 4 amino acid substitutions, relative to Formula II (SEQ ID NO: 1), wherein said substitution(s) can take place in any of the positions 1 to 34, preferably said substitution(s) take place in position 1, 3, 12, 17, 20, 24, 27, 28, 33 and/or 34. The present invention encompasses variants of the GLP-1-/GIP-/amylin-receptor tri-agonist, as disclosed herein, wherein peptide Z1 may comprise 1, 2, 3 or 4 amino acid substitution(s) relative to Formula II (SEQ ID NO: 1).

Preferred substitutions include a conservative substitution, those which, instead of the amino acid residue, which appears in the sequence, comprises an amino acid with similar biochemical properties or a structural analogue of the amino acid residue.

The peptide Z2 disclosed herein may have a maximum of 10 amino acid substitution, relative to Formula III (SEQ ID NO: 2), wherein said substitution(s) can take place in any of the positions 1 to 32, preferably said substitution(s) take place in position 2, 3, 7, 8, 9, 10, 15, 18, 20, 21, 22, 24 and/or 31. The present invention encompasses variants of the GLP-1-/GIP-/amylin-receptor tri-agonist, as disclosed herein, wherein peptide Z2 may comprise 1, 2, 3, 4, 5, 6, 7, 8, 9 or 10 amino acid substitution(s) relative to Formula III (SEQ ID NO: 2). Preferred substitutions include a conservative substitution, as above explained.

In some embodiments of the present invention the peptide Z1 comprises an amino acid sequence which has at least 87, 88, 89, 90, 91, 92, 93, 94, 95, 96, 97, 98 or 99% identity (i.e., a sequence identity) to Formula II (SEQ ID NO: 1), and the peptide Z2 comprises an amino acid sequence which has at least 70, 71, 72, 73, 74, 75, 76, 77, 78, 79, 80, 81, 82, 83, 84, 85, 86, 87, 88, 89 90, 91, 92, 93, 94, 95, 96, 97, 98 or 99% identity (i.e., a sequence identity) relative to Formula III (SEQ ID NO: 2).

In some embodiments of the invention the GLP-1-/GIP-/amylin-receptor tri-agonist disclosed herein does not comprise a cysteine (Cys, C) residue and/or does not comprise a disulfide bridge. The term “disulfide bridge” in reference to human amylin and analogues thereof, refers to a functional group with the structure R—S—S—R′ and may also be referred to as an “SS-bond”.

The GLP-1-/GIP-/amylin-receptor tri-agonist may exhibit a variety of properties rendering it useful as a medicament, as described herein.

The present invention relates in another aspect the invention relates to a GLP-1-/GIP-/amylin-receptor tri-agonist comprising a peptide according to Formula I:

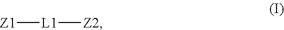

comprising one lysine (Lys, K) residue; wherein:
Z1 is a peptide having a maximum of 4 amino acid substitutions relative to Formula II (SEQ ID NO: 1):

(II)
YX₂EGTFTSDYSILLEEQAAREFIEWLLAGGPSKG,

wherein the amino acid at position X₂ represents Aib,
and wherein
Z1 comprises or consists of an amino acid sequence according to Formula X (SEQ ID NO: 161):

(X)
X₂₁X₂₂X₂₃GTFTSDYSX₂₄LLEEX₂₅AAX₂₆EFIX₂₇WLX₂₈X₂₉GGPSX₃₀X₃₁,

wherein
X₂₁ represents His (H) or Tyr (Y),
X₂₂ represents Aib,
X₂₃ represents Glu (E) or His (H),
X₂₄ represents Ile (I) or Lys (K),
X₂₅ represents Gln (Q) or Ile (i),
X₂₆ represents Arg (R) or Gln (Q),
X₂₇ represents Ala (A), Glu (E) or Gln (Q),
X₂₈ represents Leu (L) or I (Ile),
X₂₉ represents Ala (A) or Gln (Q),
X₃₀ represents Arg (R), Gly (G), Lys (K) or Ser (S),
X₃₁ represents Gly (G), Glu (E) or Lys (K);
L1 is a peptide linker; and
Z2 is a peptide comprising a C-terminal amide, and having a maximum of 10 amino acid substitutions relative to Formula III (SEQ ID NO: 2):

(III)
ASELSTAALGRLSAELHELATLPRTETGSGSP,

and
Z2 comprises or consists of an amino acid sequence according to Formula XII (SEQ ID NO:164):

(XII)
AX₃₂X₃₃LSTAX₃₄X₃₅X₃₆RLSAX₃₇LHX₃₈LX₃₉X₄₀X₄₁PX₄₂TETGSGX₄₃P,

wherein
   X₃₂ represents Gly (G) or Ser (S),
   X₃₃ represents Gln (Q), Glu (E), His (H) or Lys (K),
   X₃₄ represents Ala (A) or Gln (Q),
   X₃₅ represents Gln (Q), Leu (L) or Thr (T),
   X₃₆ represents Ala (A), Gly (G) or Gln (Q),
   X₃₇ represents Glu (E) or Lys (K),
   X₃₈ represents Arg (R), Gln (Q), Glu (E), Gly (G), His (H), Lys (K), Thr (T) or Tyr (Y),
   X₃₉ represents Ala (A) or Lys (K),
   X₄₀ represents Asp (D) or Thr (T),
   X₄₁ represents Leu (L) or Glu (E),
   X₄₂ represents Arg (R) or Lys (K),
   X₄₃ represents Ala (A) or Ser (S).

The present invention relates in another aspect to a GLP-1-/GIP-/amylin-receptor tri-agonist comprising a peptide according to Formula I:

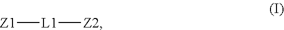

comprising one lysine (Lys, residue; wherein:
Z1 is a peptide having a maximum of 4 amino acid substitutions relative to Formula II (SEQ ID NO: 1):

(II)
YX₂EGTFTSDYSILLEEQAAREFIEWLLAGGPSKG,

wherein the amino acid at position X₂ represents Aib,
and wherein
Z1 comprises or consists of an amino acid sequence according to Formula IIa (SEQ ID NO: 3):

(IIa)
X₁X₂X₃GTFTSDYSILLEEQAAREFIEWLLAGGPSX₄X₅,

wherein
X₁ represents His (H) or Tyr (Y),
X₂ represents Aib,
X₃ represents Glu (E) or His (H),
X₄ represents Arg (R), Gly (G), Lys (K) or Ser (S),
X₅ represents Gly (G) or Lys (K);
L1 is a peptide linker; and
Z2 is a peptide comprising a C-terminal amide, and having a maximum of 10 amino acid substitutions relative to Formula III (SEQ ID NO: 2):

(III)
ASELSTAALGRLSAELHELATLPRTETGSGSP,

and
Z2 comprises or consists of an amino acid sequence according to Formula IIIa (SEQ ID NO: 4):

(IIIa)
ASX₆LSTAX₇X₈X₉RLSAX₁₀LHX₁₁LX₁₂X₁₃LPX₁₄TETGSGX₁₅P,

wherein
X₆ represents Gln (Q), Glu (E), His (H) or Lys (K),
X₇ represents Ala (A) or Gln (Q),
X₈ represents Gln (Q), Leu (L) or Thr (T),
X₉ represents Ala (A), Gly (G) or Gln (Q),
X₁₀ represents Glu (E) or Lys (K),
X₁₁ represents Arg (R), Gln (Q), Glu (E), Gly (G), His (H), Lys (K), Thr (T) or Tyr (Y),
X₁₂ represents Ala (A) or Lys (K),
X₁₃ represents Asp (D) or Thr (T),
X₁₄ represents Arg (R) or Lys (K),
X₁₅ represents Ala (A) or Ser (S).

In another aspect the invention relates to a GLP-1-/GIP-/amylin-receptor tri-agonist comprising a peptide according to Formula I:

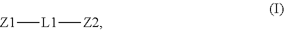

comprising one lysine (Lys, K) residue; wherein:
Z1 is a peptide having a maximum of 4 amino acid substitutions relative to Formula II (SEQ ID NO: 1):

(II)
YX₂EGTFTSDYSILLEEQAAREFIEWLLAGGPSKG,

wherein the amino acid at position X₂ represents Aib,
and wherein
Z1 comprises or consists of an amino acid sequence according to Formula Xa (SEQ ID NO: 162):

(Xa)
YX₅₁EGTFTSDYSX₅₂LLEEIAAX₅₃EFIX₅₄WLX₅₅X₅₆GGPSSX₅₇,

wherein
X₅₁ represents Aib,
X₅₂ represents Ile (I) or Lys (K),
X₅₃ represents Arg (R) or Gln (Q),
X₅₄ represents Ala (A), Glu (E) or Gln (Q),
X₅₅ represents Leu (L) or I (Ile),
X₅₆ represents Ala (A) or Gln (Q),
X₅₇ represents Gly (G) or Glu (E);
L1 is a peptide linker; and
Z2 is a peptide comprising a C-terminal amide, and having a maximum of 10 amino acid substitutions relative to Formula III (SEQ ID NO: 2):

(III)
ASELSTAALGRLSAELHELATLPRTETGSGSP,

and
Z2 comprises or consists of an amino acid sequence according to Formula XIIa (SEQ ID NO: 165):

(XIIa)
AX₅₈X₅₉LSTAX₆₀X₆₁X₆₂RLSAELHX₆₃LATX₆₄PRTETGSGSP,

wherein
   X₅₈ represents Gly (G) or Ser (S),
   X₅₉ represents Gln (Q), Glu (E), or His (H),
   X₆₀ represents Ala (A) or Gln (Q),
   X₆₁ represents Leu (L) or Thr (T),
   X₆₂ represents Ala (A), Gly (G) or Gln (Q),
   X₆₃ represents Gln (Q), Glu (E), or Lys (K),
   X₆₄ represents Leu (L) or Glu (E).

In one embodiment the GLP-1-/GIP-/amylin-receptor tri-agonist of the present invention may comprise a peptide Z1 comprising or consisting of the amino acid sequence according to Formula IIa SEQ ID NO: 3):

(IIa)
X₁X₂X₃GTFTSDYSILLEEQAaREFIEWLLAGGPSX₄X₅,

wherein
X₁X₂X₃ represents YAibE (Tyr-Aib-Glu) or HAibH (His-Aib-His)
X₄ represents Arg (R), Gly (G) or Ser (S),
X₅ represents Gly (G).

In one embodiment the GLP-1-/GIP-/amylin-receptor tri-agonist of the present invention may comprise a peptide Z1 comprising or consisting of the amino acid sequence according to Formula IIa SEQ ID NO: 3):

(IIa)
X₁X₂X₃GTFTSDYSILLEEQAaREFIEWLLAGGPSX₄X₅,

wherein
X₁ represents His (H),
X₂ represents Aib,
X₃ represents His (H),
X₄ represents Lys (K),
X₅ represents Gly (G).

In one embodiment the GLP-1-/GIP-/amylin-receptor tri-agonist of the present invention may comprise a peptide Z1 comprising or consisting of the amino acid sequence according to Formula V (SEQ ID NO: 6):

YX₂EGTFTSDYSILLEEQAAREFIEWLLAGGPSX₄G (V),

wherein
X₂ represents Aib,
X₄ is Arg (R) or Ser (S).

In one embodiment the GLP-1-/GIP-/amylin-receptor tri-agonist of the present invention may comprise a peptide Z1 comprising or consisting of the amino acid sequence according to Formula VI (SEQ ID NO: 7):

(VI)
YX₂EGTFTSDYSILLEEQAAREFIEWLLAGGPSSG

wherein X₂ represents Aib.

In some embodiments of the present invention the peptide Z1 may comprise an amino acid sequence which has at least 90, 91, 92 93, 94, 95, 96, 97, 98 or 99% identity relative to Formula VI (SEQ ID NO: 7).

In one embodiment the GLP-1-/GIP-/amylin-receptor tri-agonist of the present invention may comprise a peptide Z1 comprising or consisting of the amino acid sequence according to Formula Xa (SEQ ID NO: 162):

(Xa)
YX₅₁EGTFTSDYSX₅₂LLEEIAAX₅₃EFIX₅₄WLX₅₅X₅₆GGPSSX₅₇,

wherein
X₅₁ represents Aib,
X₅₂ represents Ile (I) or Lys (K),
X₅₃ represents Arg (R),
X₅₄ represents Glu (E),
X₅₅ represents Leu (L) or I (Ile),
X₅₆ represents Ala (A),
X₅₇ represents Gly (G) or Glu (E).

In one embodiment the GLP-1-/GIP-/amylin-receptor tri-agonist of the present invention may comprise a peptide Z1 comprising or consisting of the amino acid sequence according to Formula Xa (SEQ ID NO: 162):

(Xa)
YX₅₁EGTFTSDYSX₅₂LLEEIAAX₅₃EFIX₅₄WLX₅₅X₅₆GGPSSX₅₇,

wherein
X₅₁ represents Aib,
X₅₂ represents Ile (I) or Lys (K),
X₅₃ represents Arg (R),
X₅₄ represents Glu (E),
X₅₅ represents Leu (L),
X₅₆ represents Ala (A),
X₅₇ represents Gly (G).

In one embodiment the GLP-1-/GIP-/amylin-receptor tri-agonist of the present invention may comprise a peptide Z1 comprising or consisting of the amino acid sequence according to Formula XI (SEQ ID NO: 163):

(XI)
YX₅₁EGTFTSDYSX₅₂LLEEIAAREFIEWLLAGGPSSG,

wherein
X₅₁ represents Aib,
X₅₂ represents Ile (I) or Lys (K).

In one embodiment peptide Z2 may comprise or consist of the amino acid sequence according to Formula IIIa (SEQ ID NO: 4):

(IIIa)
ASX₆LSTAX₇X₈X₉RLSAX₁₀LHX₁₁LX₁₂X₁₃LPX₁₄TETGSGX₁₅P,

wherein
X₆ represents Lys (K),
X₇ represents Ala (A) or Gln (Q),
X₈ represents Gln (Q), Leu (L) or Thr (T),
X₉ represents Ala (A), Gly (G) or Gln (Q),
X₁₀ represents Glu (E),
X₁₁ represents Arg (R), Gln (Q), Glu (E), Gly (G), His (H), Thr (T) or Tyr (Y),
X₁₂ represents Ala (A),
X₁₃ represents Asp (D) or Thr (T),
X₁₄ represents Arg (R),
X₁₅ represents Ala (A) or Ser (S);
   or
X₆ represents Gln (Q), Glu (E), or His (H),
X₇ represents Ala (A) or Gln (Q),
X₈ represents Gln (Q), Leu (L) or Thr (T),
X₉ represents Ala (A), Gly (G) or Gln (Q),
X₁₀ represents Lys (K),
X₁₁ represents Arg (R), Gln (Q), Glu (E), Gly (G), His (H), Thr (T) or Tyr (Y),
X₁₂ represents Ala (A),
X₁₃ represents Asp (D) or Thr (T),
X₁₄ represents Arg (R),
X₁₅ represents Ala (A) or Ser (S);
   or
X₆ represents Gln (Q), Glu (E) or His (H),
X₇ represents Ala (A) or Gln (Q),
X₈ represents Gln (Q), Leu (L) or Thr (T),
X₉ represents Ala (A), Gly (G) or Gln (Q),
X₁₀ represents Glu (E),
X₁₁ represents Lys (K),
X₁₂ represents Ala (A),
X₁₃ represents Asp (D) or Thr (T),
X₁₄ represents Arg (R),
X₁₅ represents Ala (A) or Ser (S);
   or
X₆ represents Gln (Q), Glu (E) or His (H),
X₇ represents Ala (A) or Gln (Q),
X₈ represents Gln (Q), Leu (L) or Thr (T),
X₉ represents Ala (A), Gly (G) or Gln (Q),
X₁₀ represents Glu (E),
X₁₁ represents Arg (R), Gln (Q), Glu (E), Gly (G), His (H), Thr (T) or Tyr (Y),
X₁₂ represents Lys (K),
X₁₃ represents Asp (D) or Thr (T),
X₁₄ represents Arg (R),
X₁₅ represents Ala (A) or Ser (S);
   or
X₆ represents Gln (Q), Glu (E) or His (H),
X₇ represents Ala (A) or Gln (Q),
X₈ represents Gln (Q), Leu (L) or Thr (T),
X₉ represents Ala (A), Gly (G) or Gln (Q),
X₁₀ represents Glu (E),
X₁₁ represents Arg (R), Gln (Q), Glu (E), Gly (G), His (H), Thr (T) or Tyr (Y),
X₁₂ represents Ala (A),
X₁₃ represents Asp (D) or Thr (T),
X₁₄ represents Lys (K),
X₁₅ represents Ala (A) or Ser (S).

In one embodiment peptide Z2 may comprise or consist of the amino acid sequence according to Formula IIIa (SEQ ID NO: 4):

(IIIa)
ASX₆LSTAX₇X₈X₉RLSAX₁₀LHX₁₁LX₁₂X₁₃LPX₁₄TETGSGX₁₅P,

wherein
X₆ represents His (H),
X₇ represents Gln (Q),
X₈ represents Thr (T),
X₉ represents Gln (Q),
X₁₀ represents Glu (E),
X₁₁ represents Arg (R) or Lys (K),
X₁₂ represents Ala (A) or Lys (K),
X₁₃ represents Thr (T),
X₁₄ represents Arg (R) or Lys (K),
X₁₅ represents Ser (S).

In one embodiment peptide Z2 may comprise or consist of the amino acid sequence according to Formula Iiia (SEQ ID NO: 4):

(IIIa)
ASX₆LSTAX₇X₈X₉RLSAX₁₀LHX₁₁LX₁₂X₁₃LPX₁₄TETGSGX₁₅P,

wherein
X₆ represents His (H),
X₇ represents Ala (A) or Gln (Q),
X₈ represents Leu (L) or Thr (T),
X₉ represents Ala (A), Gly (G) or Gln (Q),
X₁₀ represents Glu (E),
X₁₁ represents Arg (R) or Gln (Q),
X₁₂ represents Lys (K),
X₁₃ represents Thr (T),
X₁₄ represents Arg (R),
X₁₅ represents Ser (S).

In one embodiment peptide Z2 may comprise or consist of the amino acid sequence according to Formula Iiia (SEQ ID NO: 4):

(IIIa)
ASX₆LSTAX₇X₈X₉RLSAX₁₀LHX₁₁LX₁₂X₁₃LPX₁₄TETGSGX₁₅P,

wherein
X₆ represents His (H),
X₇ represents Ala (A) or Gln (Q),
X₈ represents Leu (L) or Thr (T),
X₉ represents Ala (A), Gly (G) or Gln (Q),
X₁₀ represents Glu (E),
X₁₁ represents Arg (R),
X₁₂ represents Ala (A),
X₁₃ represents Thr (T),
X₁₄ represents Lys (K),
X₁₅ represents Ser (S).

In one embodiment peptide Z2 may comprise or consist of the amino acid sequence according to Formula IIIa (SEQ ID NO: 4):

(IIIa)
ASX₆LSTAX₇X₈X₉RLSAX₁₀LHX₁₁LX₁₂X₁₃LPX₁₄TETGSGX₁₅P,

wherein
X₆ represents Gln (Q), Glu (E), His (H) or Lys (K),
X₇ represents Ala (A) or Gln (Q),
X₈ represents Gln (Q), Leu (L) or Thr (T),
X₉ represents Ala (A), Gly (G) or Gln (Q),
X₁₀ represents Glu (E) or Lys (K),
X₁₁ represents Arg (R), Gln (Q), Glu (E), Gly (G), His (H), Lys (K), Thr (T) or Tyr (Y),
X₁₂ represents Ala (A) or Lys (K),
X₁₃ represents Asp (D) or Thr (T),
X₁₄ represents Arg (R) or Lys (K),
X₁₅ represents Ala (A) or Ser (S).

In one embodiment peptide Z2 may comprise or consist of the amino acid sequence according to Formula IIIa (SEQ ID NO: 4):

(IIIa)
ASX₆LSTAX₇X₈X₉RLSAX₁₀LHX₁₁LX₁₂X₁₃LPX₁₄TETGSGX₁₅P,

wherein
X₆ represents Glu (E) or His (H),
X₇ represents Ala (A) or Gln (Q),
X₈ represents Leu (L) or Thr (T),
X₉ represents Ala (A), Gly (G) or Gln (Q),
X₁₀ represents Glu (E),
X₁₁ represents Arg (R), Gln (Q) or Glu (E),
X₁₂ represents Ala (A),
X₁₃ represents Asp (D) or Thr (T),
X₁₄ represents Arg (R),
X₁₅ represents Ser (S).

In one embodiment peptide Z2 may comprise or consist of the amino acid sequence according to Formula IIIa (SEQ ID NO: 4):

(IIIa)
ASX₆LSTAX₇X₈X₉RLSAX₁₀LHX₁₁LX₁₂X₁₃LPX₁₄TETGSGX₁₅P,

wherein
X₆ represents Glu (E) or His (H),
X₇ represents Ala (A) or Gln (Q),
X₈ represents Leu (L) or Thr (T),
X₉ represents Ala (A) or Gly (G),
X₁₀ represents Glu (E),
X₁₁ represents Arg (R), Gln (Q) or Glu (E),
X₁₂ represents Ala (A),
X₁₃ represents Thr (T),
X₁₄ represents Arg (R),
X₁₅ represents Ser (S).

In one embodiment peptide Z2 may comprise or consist of the amino acid sequence according to according to Formula VII (SEQ ID NO: 8):

(VII)
ASHLSTAQTQRLSAKLHRLATLPRTETGSGSP.

In one embodiment peptide Z2 may comprise or consist of the amino acid sequence according to Formula VIII (SEQ ID NO: 9):

(VIII)
ASHLSTAQTQRLSAELHKLATLPRTETGSGSP.

In some embodiments of the present invention the peptide Z2 may comprise an amino acid sequence which has at least 90, 91, 92 93, 94, 95, 96, 97, 98 or 99% identity relative to Formula VII (SEQ ID NO: 8) or Formula VIII (SEQ ID NO: 9).

In one embodiment the GLP-1-/GIP-/amylin-receptor tri-agonist of the present invention may comprise a peptide Z2 comprising or consisting of the amino acid sequence according to Formula XIIa (SEQ ID NO: 165):

(XIIa)
AX₅₈X₅₉LSTAX₆₀X₆₁X₆₂RLSAELHX₆₃LATX₆₄PRTETGSGSP,

wherein
X₅₈ represents Ser (5),
X₅₉ represents His (H),
X₆₀ represents Gln (Q),
X₆₁ represents Thr (T),
X₆₂ represents Gln (Q),
X₆₃ represents Lys (K),
X₆₄ represents Leu (L).

In one embodiment the GLP-1-/GIP-/amylin-receptor tri-agonist of the present invention may comprise a peptide Z2 comprising or consisting of the amino acid sequence according to Formula XIII (SEQ ID NO: 166):

(XIII)
ASX₅₉LSTAQTQRLSAELHKLATLPRTETGSGSP,

wherein
X₅₉ represents Glu (E) or His (H).

In one embodiment the GLP-1-/GIP-/amylin-receptor tri-agonist of the present invention may comprise a peptide Z1 comprising or consisting of an amino acid sequence which has at least 90, 91, 92 93, 94, 95, 96, 97, 98 or 99% identity relative to Formula VI (SEQ ID NO: 7), and a peptide Z2 comprising or consisting of an amino acid sequence which has at least 90, 91, 92 93, 94, 95, 96, 97, 98 or 99% identity relative to Formula VII (SEQ ID NO: 8) or Formula VIII (SEQ ID NO: 9).

In one embodiment the GLP-1-/GIP-/amylin-receptor tri-agonist of the present invention may comprise a peptide Z1 comprising or consisting of the amino acid sequence according to Formula IIa SEQ ID NO: 3):

(IIa)
X₁X₂X₃GTFTSDYSILLEEQAAREFIEWLLAGGPSX₄X₅,

wherein
X₁ represents His (H) or Tyr (Y),
X₂ represents Aib,
X₃ represents Glu (E) or His (H),
X₄ represents Arg (R), Gly (G) or Ser (S),
X₅ represents Gly (G); and
   a peptide Z2 comprising or consisting of the amino acid sequence according to Formula IIIa (SEQ ID NO: 4):

(IIIa)
ASX₆LSTAX₇X₈X₉RLSAX₁₀LHX₁₁LX₁₂X₁₃LPX₁₄TETGSGX₁₅P,

wherein
X₆ represents Gln (Q), Glu (E), His (H) or Lys (K),
X₇ represents Ala (A) or Gln (Q),
X₈ represents Gln (Q), Leu (L) or Thr (T),
X₉ represents Ala (A), Gly (G) or Gln (Q),
X₁₀ represents Glu (E) or Lys (K),
X₁₁ represents Arg (R), Gln (Q), Glu (E), Gly (G), His (H), Lys (K), Thr (T) or Tyr (Y),
X₁₂ represents Ala (A) or Lys (K),
X₁₃ represents Asp (D) or Thr (T),
X₁₄ represents Arg (R) or Lys (K),
X₁₅ represents Ala (A) or Ser (S).

In one embodiment the GLP-1-/GIP-/amylin-receptor tri-agonist of the present invention may comprise a peptide Z1 comprising or consisting of the amino acid sequence according to Formula IIa (SEQ ID NO: 3):

(IIa)
X₁X₂X₃GTFTSDYSILLEEQAAREFIEWLLAGGPSX₄X₅,

wherein
X₁ represents His (H) or Tyr (Y),
X₂ represents Aib,
X₃ represents Glu (E) or His (H),
X₄ represents Arg (R), Gly (G) or Ser (S),
X₅ represents Gly (G); and
   a peptide Z2 comprising or consisting of the amino acid sequence according to Formula IIIa (SEQ ID NO: 4):

(IIIa)
ASX₆LSTAX₇X₈X₉RLSAX₁₀LHX₁₁LX₁₂X₁₃LPX₁₄TETGSGX₁₅P,

wherein
X₆ represents Glu (E) or His (H),
X₇ represents Ala (A) or Gln (Q),
X₈ represents Gln (Q), Leu (L) or Thr (T),
X₉ represents Ala (A), Gly (G) or Gln (Q),
X₁₀ represents Glu (E) or Lys (K),
X₁₁ represents Arg (R), Gln (Q) or Lys (K),
X₁₂ represents Ala (A) or Lys (K),
X₁₃ represents Thr (T),
X₁₄ represents Arg (R) or Lys (K),
X₁₅ represents Ala (A) or Ser (S).

In one embodiment the GLP-1-/GIP-/amylin-receptor tri-agonist of the present invention may comprise a peptide Z1 comprising or consisting of the amino acid sequence according to Formula IIa SEQ ID NO: 3):

(IIa)
X₁X₂X₃GTFTSDYSILLEEQAAREFIEWLLAGGPSX₄X₅,

wherein
X₁ represents His (H) or Tyr (Y),
X₂ represents Aib,
X₃ represents Glu (E) or His (H),
X₄ represents Arg (R), Gly (G) or Ser (S),
X₅ represents Gly (G); and
   a peptide Z2 comprises or consists of the amino acid sequence according to Formula IIIa (SEQ ID NO: 4):

(IIIa)
ASX₆LSTAX₇X₈X₉RLSAX₁₀LHX₁₁LX₁₂X₁₃LPX₁₄TETGSGX₁₅P,

wherein
X₆ represents Glu (E) or His (H),
X₇ represents Ala (A) or Gln (Q),
X₈ represents Gln (Q), Leu (L) or Thr (T),
X₉ represents Ala (A), Gly (G) or Gln (Q),
X₁₀ represents Glu (E),
X₁₁ represents Lys (K),
X₁₂ represents Ala (A),
X₁₃ represents Thr (T),
X₁₄ represents Arg (R),
X₁₅ represents Ala (A) or Ser (S).

In one embodiment the GLP-1-/GIP-/amylin-receptor tri-agonist of the present invention may comprise a peptide Z1 comprising or consisting of the amino acid sequence according to Formula Xa (SEQ ID NO: 162):

(Xa)
YX₅₁EGTFTSDYSX₅₂LLEEIAAX_{53E}FIX₅₄WLX₅₅X₅₆GGPSSX₅₇,

wherein
X₅₁ represents Aib,
X₅₂ represents Ile (I) or Lys (K),
X₅₃ represents Arg (R),
X₅₄ represents Glu (E),
X₅₅ represents Leu (L) or I (Ile),
X₅₆ represents Ala (A),
X₅₇ represents Gly (G) or Glu (E); and
   a peptide Z2 comprising or consisting of the amino acid sequence according to Formula XIIa (SEQ ID NO: 165):

(XIIa)
AX₅₈X₅₉LSTAX₆₀X₆₁X₆₂RLSAELHX₆₃LATX₆₄PRTETGSGSP,

wherein
X₅₈ represents Ser (S),
X₅₉ represents His (H),
X₆₀ represents Gln (Q),
X₅₁ represents Thr (T),
X₆₂ represents Gln (Q),
X₆₃ represents Lys (K),
X₆₄ represents Leu (L).

In one embodiment the invention relates to a GLP-1-/GIP-/amylin-receptor tri-agonist comprising a peptide, wherein the peptide comprises or consists of an amino acid sequence selected from the group consisting of SEQ ID NOs 20 to 124 and 170 to 242, and wherein X represents Aib.

In a particular embodiment the invention relates to a GLP-1-/GIP-/amylin-receptor tri-agonist, wherein the amino acid sequence of the peptide comprises or consists of

(SEQ ID NO: 62)
YXEGTFTSDYSILLEEQAAREFIEWLLAGGPSSGAGQAPGQAPGASHLS
TAQTQRLSAELHKLATLPRTETGSGSP, wherein X represents
Aib;
or
(SEQ ID NO: 65)
YXEGTFTSDYSILLEEQAAREFIEWLLAGGPSSGAGQAPGQAPGASHLS
TAQTQRLSAKLHRLATLPRTETGSGSP, wherein X represents
Aib;
or
(SEQ ID NO: 68)
YXEGTFTSDYSILLEEQAAREFIEWLLAGGPSSGAGQAPGQAPLASHLS
TAQTQRLSAELHKLATLPRTETGSGSP, wherein X represents
Aib;
or
(SEQ ID NO: 78)
YXEGTFTSDYSILLEEQAAREFIEWLLAGGPSRGEASHLSTAQTQRLSA
ELHKLATLPRTETGSGSP, wherein X represents Aib;
or
(SEQ ID NO: 87)
YXEGTFTSDYSILLEEQAAREFIEWLLAGGPSSGAGQAPGQAPLASHLS
TAQTQRLSAELHKLATLPRTETGSGSP, wherein X represents
Aib;
or
(SEQ ID NO: 111)
HXHGTFTSDYSILLEEQAAREFIEWLLAGGPSKGAPPPSGGGEASHLST
AQTARLSAELHQLATLPRTETGSGSP, wherein X represents
Aib;
or
(SEQ ID NO: 170)
YXEGTFTSDYSILLEEIAAREFIEWLLAGGPSSGAGQAPGQAPGASHLS
TAQTQRLSAELHKLATLPRTETGSGSP; wherein X represents
Aib.

In one aspect of the invention relates to a GLP-1-/GIP-/amylin-receptor tri-agonist which is capable of activating the human GIP, GLP-1, and amylin receptors in vitro.

When tested as described in “GLP-1 receptor assay” (preferably method A; in the absence of HSA), the GLP-1-/GIP-/amylin-receptor tri-agonist disclosed herein may have an EC₅₀ value of less than 125 pM, preferably less than 100 pM, such as less than 75 pM, even more preferably less than 50 pM, such as less than 40 pM, and most preferably less than 30 pM, such as less than 20 pM, such as less than 10 pM, such as less than 5 pM.

When tested as described in “GIP receptor assay” (preferably method A; in the absence of HSA), the GLP-1-/GIP-/amylin-receptor tri-agonist disclosed herein may have an EC₅₀ value of less than 125 pM, preferably less than 100 pM, such as less than 75 pM, even more preferably less than 50 pM, such as less than 40 pM, and most preferably less than 30 pM, such as less than 20 pM, such as less than 10 pM, such as less than 5 pM.

When tested as described in “Amylin receptor assay” (preferably method A; in the absence of HSA), the GLP-1-/GIP-/amylin-receptor tri-agonist disclosed herein may have an EC₅₀ value of less than 125 pM, preferably less than 100 pM, such as less than 75 pM, even more preferably less than 50 pM, such as less than 40 pM, and most preferably less than 30 pM, such as less than 20 pM, such as less than 10 pM, such as less than 5 pM. The GLP-1-/GIP-/amylin-receptor tri-agonist disclosed herein agonise, or activate, the amylin receptor. The GLP-1-/GIP-/amylin-receptor tri-agonist disclosed herein may be tested for amylin activity as described in Example 4.

The more potent the compound, the lower its EC₅₀ value. The GLP-1-/GIP-/amylin-receptor tri-agonist may have an EC₅₀ in a human GLP-1 receptor functional assay (see Example 4) of about 100 pM or less. The GLP-1-/GIP-/amylin-receptor tri-agonist may have an EC₅₀ in a human GLP-1 receptor functional assay of about 90 pM or less. The GLP-1-/GIP-/amylin-receptor tri-agonist may have an EC₅₀ in a human GLP-1 receptor functional assay of about 80 pM or less. The GLP-1-/GIP-/amylin-receptor tri-agonist may have an EC₅₀ in a human GLP-1 receptor functional assay of about 75 pM or less. The GLP-1-/GIP-/amylin-receptor tri-agonist may have an EC₅₀ in a human GLP-1 receptor functional assay of about 70 pM or less. The GLP-1-/GIP-/amylin-receptor tri-agonist may have an EC₅₀ in a human GLP-1 receptor functional assay of about 60 pM or less. The GLP-1-/GIP-/amylin-receptor tri-agonist may have an EC₅₀ in a human GLP-1 receptor functional assay of about 50 pM or less. The GLP-1-/GIP-/amylin-receptor tri-agonist may have an EC₅₀ in a human GLP-1 receptor functional assay of about 40 pM or less. The GLP-1-/GIP-/amylin-receptor tri-agonist may have an EC₅₀ in a human GLP-1 receptor functional assay of about 30 pM or less. The GLP-1-/GIP-/amylin-receptor tri-agonist may have an EC₅ in a human GLP-1 receptor functional assay of about 25 pM or less. The GLP-1-/GIP-/amylin-receptor tri-agonist may have an EC₅₀ in a human GLP-1 receptor functional assay of about 20 pM or less. The GLP-1-/GIP-/amylin-receptor tri-agonist may have an EC₅₀ in a human GLP-1 receptor functional assay of about 15 pM or less. The GLP-1-/GIP-/amylin-receptor tri-agonist may have an EC₅₀ in a human GLP-1 receptor functional assay of about 10 pM or less. The GLP-1-/GIP-/amylin-receptor tri-agonist may have an EC₅₀ in a human GLP-1 receptor functional assay of about 5 pM or less. The GLP-1-/GIP-/amylin-receptor tri-agonist disclosed herein may have a similar potency as that of semaglutide or tirzepatide.

The GLP-1-/GIP-/amylin-receptor tri-agonist may have an EC₅₀ in a human GIP receptor functional assay (see Example 4) of about 125 pM or less. The GLP-1-/GIP-/amylin-receptor tri-agonist may have an EC₅₀ in a human GIP receptor functional assay of about 100 pM or less. The GLP-1-/GIP-/amylin-receptor tri-agonist may have an EC₅₀ in a human GIP receptor functional assay of about 90 pM or less. The GLP-1-/GIP-/amylin-receptor tri-agonist may have an EC₅₀ in a human GIP receptor functional assay of about 80 pM or less. The GLP-1-/GIP-/amylin-receptor tri-agonist may have an EC₅₀ in a human GIP receptor functional assay of about 75 pM or less. The GLP-1-/GIP-/amylin-receptor tri-agonist may have an EC₅₀ in a human GIP receptor functional assay of about 70 pM or less. The GLP-1-/GIP-/amylin-receptor tri-agonist may have an EC₅₀ in a human GIP receptor functional assay of about 60 pM or less. The GLP-1-/GIP-/amylin-receptor tri-agonist may have an EC₅₀ in a human GIP receptor functional assay of about 50 pM or less. The GLP-1-/GIP-/amylin-receptor tri-agonist may have an EC₅₀ in a human GIP receptor functional assay of about 40 pM or less. The GLP-1-/GIP-/amylin-receptor tri-agonist may have an EC₅₀ in a human GIP receptor functional assay of about 30 pM or less. The GLP-1-/GIP-/amylin-receptor tri-agonist may have an EC₅₀ in a human GIP receptor functional assay of about 25 pM or less. The GLP-1-/GIP-/amylin-receptor tri-agonist may have an EC₅₀ in a human GIP receptor functional assay of about 20 pM or less. The GLP-1-/GIP-/amylin-receptor tri-agonist may have an EC₅₀ in a human GIP receptor functional assay of about 15 pM or less. The GLP-1-/GIP-/amylin-receptor tri-agonist may have an EC₅₀ in a human GIP receptor functional assay of about 10 pM or less. The GLP-1-/GIP-/amylin-receptor tri-agonist may have an EC₅₀ in a human GIP receptor functional assay of about 9 pM or less. The GLP-1-/GIP-/amylin-receptor tri-agonist may have an EC₅₀ in a human GIP receptor functional assay of about 8 pM or less. The GLP-1-/GIP-/amylin-receptor tri-agonist may have an EC₅₀ in a human GIP receptor functional assay of about 7 pM or less. The GLP-1-/GIP-/amylin-receptor tri-agonist may have an EC₅₀ in a human GIP receptor functional assay of about 6 pM or less. The GLP-1-/GIP-/amylin-receptor tri-agonist may have an EC₅ in a human GIP receptor functional assay of about 5 pM or less. The GLP-1-/GIP-/amylin-receptor tri-agonist disclosed herein may have a similar potency as that of tirzepatide.

The GLP-1-/GIP-/amylin-receptor tri-agonist may have an EC₅₀ in a human amylin receptor functional assay (see Example 4) of about 125 pM or less. The GLP-1-/GIP-/amylin-receptor tri-agonist may have an EC₅₀ in a human amylin receptor functional assay of about 100 pM or less. The GLP-1-/GIP-/amylin-receptor tri-agonist may have an EC₅₀ in a human amylin receptor functional assay of about 90 pM or less. The GLP-1-/GIP-/amylin-receptor tri-agonist may have an EC₅₀ in a human amylin receptor functional assay of about 80 pM or less. The GLP-1-/GIP-/amylin-receptor tri-agonist may have an EC₅₀ in a human amylin receptor functional assay of about 75 pM or less. The GLP-1-/GIP-/amylin-receptor tri-agonist may have an EC₅₀ in a human amylin receptor functional assay of about 70 pM or less. The GLP-1-/GIP-/amylin-receptor tri-agonist may have an EC₅₀ in a human amylin receptor functional assay of about 60 pM or less. The GLP-1-/GIP-/amylin-receptor tri-agonist may have an EC₅₀ in a human amylin receptor functional assay of about 50 pM or less. The GLP-1-/GIP-/amylin-receptor tri-agonist may have an EC₅₀ in a human amylin receptor functional assay of about 40 pM or less. The GLP-1-/GIP-/amylin-receptor tri-agonist may have an EC₅₀ in a human amylin receptor functional assay of about 30 pM or less. The GLP-1-/GIP-/amylin-receptor tri-agonist may have an EC₅₀ in a human amylin receptor functional assay of about 25 pM or less. The GLP-1-/GIP-/amylin-receptor tri-agonist may have an EC₅₀ in a human amylin receptor functional assay of about 20 pM or less. The GLP-1-/GIP-/amylin-receptor tri-agonist may have an EC₅₀ in a human amylin receptor functional assay of about 15 pM or less. The GLP-1-/GIP-/amylin-receptor tri-agonist may have an EC₅₀ in a human amylin receptor functional assay of about 10 pM or less. The GLP-1-/GIP-/amylin-receptor tri-agonist may have an EC₅₀ in a human amylin receptor functional assay of about 9 pM or less. The GLP-1-/GIP-/amylin-receptor tri-agonist may have an EC₅₀ in a human amylin receptor functional assay of about 8 pM or less. The GLP-1-/GIP-/amylin-receptor tri-agonist may have an EC₅₀ in a human amylin receptor functional assay of about 7 pM or less. The GLP-1-/GIP-/amylin-receptor tri-agonist may have an EC₅₀ in a human amylin receptor functional assay of about 6 pM or less. The GLP-1-/GIP-/amylin-receptor tri-agonist may have an EC₅₀ in a human amylin receptor functional assay of about 5 pM or less. The GLP-1-/GIP-/amylin-receptor tri-agonist disclosed herein may have a similar potency as that of cagrilintide.

In another aspect the invention relates to a balanced GLP-1-/GIP-/amylin-receptor tri-agonist which activates the human GIP, GLP-1, and amylin receptors in vitro, when measured without HSA in assays as described in Example 4, and which has a potency ratio of less than 50.

### Peptide Linker

The GLP-1-/GIP-/amylin-receptor tri-agonist peptide backbone disclosed herein comprise a peptide linker L1, which may comprise 1 to 14 amino acid residues, in particularly canonical amino acid residues. The peptide linker may comprise 1 to 10 amino acid residues, in particularly canonical amino acid residues, such as 2 to 10, 3 to 10, 4 to 10, 5 to 10, 6 to 10, 7 to 10, 8 to 10, 9 to 10, 1 to 9, 1 to 8, 1 to 7, 1 to 6, 1 to 5, 1 to 4, or 1 to 3 amino acid residues. Specifically, the peptide linker may comprise 1, 2, 3, 4, 5, 6, 7, 8, 9 or 10 amino acid residues, in particularly canonical amino acid residues.

The peptide linker L1 may be represented b Formula IV:

(IV)
X₁X₂X₃X₄X₅X₆X₇X₈X₉X₁₀X₁₁X₁₂X₁₃X₁₄,

wherein any of X₁₋₁₄ is independently selected from any naturally occurring, or canonical, amino acid residue(s), and wherein any of X₂₋₁₄ may be absent.

The GLP-1-/GIP-/amylin-receptor tri-agonist peptide backbone disclosed herein comprise a peptide linker L1, wherein the peptide linker L1 comprises or consists of the amino acid sequence according to Formula IV. Thus, the GLP-1-/GIP-/amylin-receptor tri-agonist may comprise a peptide or consist of a peptide according to the amino acid sequence of SEQ ID No: 5.

Any of X₁₋₁₄ may be selected from any nonaromatic amino acid residue. Any of X₁₋₁₄ may be a charged amino acid. Any of X₁₋₁₄ may be a polar amino acid. Any of X₁₋₁₄ may be a hydrophobic amino acid.

Any of X₁₋₁₄ may be independently selected from the group consisting of alanine (Ala, A), glutamic acid (Glu, E), glutamine (Gln, Q), glycine (Gly, G), leucine (Leu, L), phenylalanine (Phe, F), proline (Pro, P), serine (Ser, S), threonine (Thr, T), valine (Val, V) and asparagine (Asn, N). Preferably any of X₁₋₁₄ may be selected from the group consisting of alanine (Ala, A), glutamic acid (Glu, E), glutamine (Gln, Q), glycine (Gly, G), leucine (Leu, L) and proline (Pro, P).

In one embodiment the peptide linker L1 may be represented by Formula IV, or comprises or consists of the amino acid sequence according to Formula IV:

(IV)
X₁X₂X₃X₄X₅X₆X₇X₈X₉X₁₀X₁₁X₁₂X₁₃X₁₄,

wherein
X₁ represents Ala (A), Glu (E), Gly (G),
X₂ represents Gln (Q), Glu (E), Gly (G), Leu (L), Pro (P) or is absent,
X₃ represents Ala (A), Gln (Q), Glu (E), Gly (G), Pro (P) or is absent,
X₄ represents Ala (A), Gln (Q), Glu (E), Gly (G), Pro (P) or is absent,
X₅ represents Glu (E), Gly (G), Pro (P), Ser (S), Thr (T) or is absent,
X₆ represents Glu (E), Gly (G), Leu (L), Gln (Q) or is absent,
X₇ represents Ala (A), Gln (Q), Glu (E), Gly (G), Phe (F) or is absent,
X₈ represents Ala (A), Gln (Q), Glu (E), Gly (G), Thr (T), Pro (P), Val (V) or is absent,
X₉ represents Glu (E), Asn (N), Pro (P), Thr (T) or is absent,
X₁₀ represents Ala (A), Gln (Q), Glu (E), Gly (G), Leu (L), Pro (P), Ser (S), Val (V) or is absent,
X₁₁ represents Ala (A) or is absent,
X₁₂ represents Gln (Q) or is absent,
X₁₃ represents Thr (T) or is absent,
X₁₄ represents Leu (L) or is absent.

The peptide linker L1 may be any one of the peptide linkers represented by SEQ ID NOs 125-159. The peptide linker L1 may be any one of the peptide linkers listed in Table 2.

TABLE 2
Peptide linkers L1

| SEQ | Peptide | SEQ | Peptide |
|---|---|---|---|
| ID NO | linker | ID NO | linker |
| — | A | 132 | ALAQTLFVNQ |
| — | E | 133 | ALAQTLGTNE |
| — | G | 134 | ALQAPGQAPG |
| — | AE | 135 | ALQAPGQAPL |
| — | GE | 136 | AGQAPGQAPG |
| 125 | APPE | 137 | AGQAPGQAPL |
| 126 | GGGE | 138 | GGGEGGGEGE |
| 127 | AGQAPG | 139 | GQAPGQAPGE |
| 128 | APPPSGGG | 140 | GQEPGQEPGE |
| 129 | APPPSGGGE | 141 | APPPSLAQTLAQTL |
| 130 | APPPSGGGG | 150 | EGQAPGQAPG |
| 131 | ALAQTLAQTL | 151 | AGQEPGQAPG |
| — | AG | 152 | AGQAEGQAPG |
| 142 | AGGGG | 153 | AGQAPEQAPG |
| 143 | AGEAPGQAPG | 154 | AGQAPGEAPG |
| 144 | AGEAPGEAPG | 155 | AGQAPGQEPG |
| 145 | AGQAPGQAPA | 156 | AGQAPGQAEG |
| 146 | AGQAPGQAPE | 157 | AGQEPGQEPG |
| 147 | AGQAPGQAPP | 158 | AGQAPGQAP |
| 148 | AGQAPGQAPS | 159 | AGQAPGEAPL |
| 149 | AGQAPGQAPV | | |

The peptide linker L1 may be selected from the
group consisting of
A,
E,
G,
AE,
GE,
(SEQ ID NO: 125)
APPE,
(SEQ ID NO: 126)
GGGE,
(SEQ ID NO: 127)
AGQAPG,
(SEQ ID NO: 128)
APPPSGGG,
(SEQ ID NO: 129)
APPPSGGGE,
(SEQ ID NO: 130)
APPPSGGGG,
(SEQ ID NO: 131)
ALAQTLAQTL,
(SEQ ID NO: 132)
ALAQTLFVNQ,
(SEQ ID NO: 133)
ALAQTLGTNE,
(SEQ ID NO: 134)
ALQAPGQAPG,
(SEQ ID NO: 135)
ALQAPGQAPL,
(SEQ ID NO: 136)
AGQAPGQAPG,
(SEQ ID NO: 137)
AGQAPGQAPL,
(SEQ ID NO: 138)
GGGEGGGEGE,
(SEQ ID NO: 139)
GQAPGQAPGE,
(SEQ ID NO: 140)
GQEPGQEPGE
(SEQ ID NO: 141)
APPPSLAQTLAQTL,
AG,
(SEQ ID NO: 142)
AGGGG,
(SEQ ID NO: 143)
AGEAPGQAPG,
(SEQ ID NO: 144)
AGEAPGEAPG,
(SEQ ID NO: 145)
AGQAPGQAPA,
(SEQ ID NO: 146)
AGQAPGQAPE,
(SEQ ID NO: 147)
AGQAPGQAPP,
(SEQ ID NO: 148)
AGQAPGQAPS,
(SEQ ID NO: 149)
AGQAPGQAPV,
(SEQ ID NO: 150)
EGQAPGQAPG,
(SEQ ID NO: 151)
AGQEPGQAPG,
(SEQ ID NO: 152)
AGQAEGQAPG,
(SEQ ID NO: 153)
AGQAPEQAPG,
(SEQ ID NO: 154)
AGQAPGEAPG,
(SEQ ID NO: 155)
AGQAPGQEPG,
(SEQ ID NO: 156)
AGQAPGQAEG,
(SEQ ID NO: 157)
AGQEPGQEPG,
(SEQ ID NO: 158)
AGQAPGQAP
and
(SEQ ID NO: 159)
AGQAPGEAPL.
In a preferred embodiment the peptide linker L1
may be
E,
GE,
(SEQ ID NO: 129)
PPPSGGGE,
(SEQ ID NO: 136)
AGQAPGQAPG,
or
(SEQ ID NO: 137)
AGQAPGQAPL
or
(SEQ ID NO: 154)
AGQAPGEAPG

In one embodiment the backbone of the peptide Z1-L1-Z2 comprises 66 to 80 amino acid residues.

In one embodiment the backbone of the peptide Z1-L1-Z2 comprises 67, 68, 75 or 76 amino acid residues.

In one embodiment the backbone of the peptide Z1-L1-Z2 comprises 76 amino acid residues.

In one embodiment the backbone of the peptide Z1-L1-Z2 comprises or consists of an amino acid sequence according to SEQ ID NO: 5.

In one embodiment the GLP-1-/GIP-/amylin-receptor tri-agonist comprises a peptide according to Formula I: Z1-L1-Z2, wherein the peptide comprises or consists of an amino acid sequence selected from the group consisting of SEQ ID NOs 5, 20 to 124 and 170 to 242.

### Protraction Moiety

In another aspect the invention relates to a GLP-1-/GIP-/amylin-receptor tri-agonist which may further comprise a protraction moiety. In such cases the peptide is referred to as a “peptide derivative”. The addition of the term “derivative” thus means that a protraction moiety is present and a compound or compounds comprising a protraction moiety are referred to as “derivative” or “derivatives”.

The term “protraction moiety” as used herein refers to a moiety having half-life extending properties and comprising a “protractor P” and an optional “linker L_{P}”, and may be represented by the general formula “L_{P}-P”, in which L_{P} is said optional linker and P is said protractor.

The term “protractor” as used herein refers to a molecule which is capable of increasing the plasma half-life of the peptide to which it is attached. The term “protraction” thus refers to half-life extension and a protractor or protraction moiety serves the purpose of extending the plasma half-life of the peptides as disclosed herein.

Furthermore, the GLP-1-/GIP-/amylin-receptor tri-agonist of the present invention have a long plasma half-life relative to dosing interval, thus reducing the variability in steady state exposure, and thus making a once weekly administration possible. The compound disclosed herein may be orally bioavailable, hence suitable for oral administration of subjects in need thereof. Both the peptide backbone and the protraction moiety have been engineered and refined in order to achieve a compound having all of the above properties.

Each protraction moiety L_{P}-P covalently attaches to the epsilon amino group of a lysine residue in the peptide backbone of the inventive GLP-1-/GIP-/amylin-receptor tri-agonist. The protraction moiety L_{P}-P may attach to the epsilon position (i.e., amino group) of the one lysine (Lys, K) residue. The protraction moiety L_{P}-P may attach to the epsilon position (i.e., amino group) of a lysine (Lys, K) residue in the peptide Z1 portion of the peptide backbone (the “21” in Z1-L1-Z2), such as a lysine (Lys, K) residue at position X₄ or X₅ of Formula IIa (SEQ ID NO: 3) or at positions 33 or 34 of Formula IIa (SEQ ID NO: 3), or at position 12 of Formula Xa (SEQ ID NO: 162) or at position X₅₂ of Formula Xa (SEQ ID NO: 162).

The protraction moiety L_{P}-P may attach to the epsilon position (i.e., amino group) of a lysine (Lys, K) residue in the peptide linker L1 portion of the peptide backbone (the “L1” in Z1-L1-Z2).

The protraction moiety L_{P}-P may attach to the epsilon position (i.e. amino group) of a lysine (Lys, K) residue in the peptide Z2 portion of the peptide backbone (the “22” in Z1-L1-Z2), such as a lysine (Lys, K) residue at any one of positions X₆, X₁₀, X₁₁, X₁₂, or X₁₄ of Formula IIIa (SEQ ID NO: 4) or at positions 3, 15, 18, 20 or 24 of Formula IIIa (SEQ ID NO: 4), or at position 18 of Formula XIIa (SEQ ID NO: 165) or at position X₆₃ of Formula XIIa (SEQ ID NO: 165). The attachment point is generally referred to as R1.

When the optional linker L_{P} is present, the protraction moiety L_{P}-P covalently attaches to the peptide backbone via the linker L_{P}. When the linker L_{P} is absent, P covalently attaches to the peptide backbone.

The GLP-1-/GIP-/amylin-receptor tri-agonist of the present invention, as disclosed herein, comprises or consists of a peptide comprising one lysine (Lys, K) residue, to which a single protraction moiety is covalently attached/conjugated (at the epsilon amino group). A protraction moiety may consist of one protractor P. A protraction moiety may comprise one linker L_{P} and one protractor P. A protraction moiety may comprise one linker L_{P} and two or more protractors (in this case referred to as P1, P2 or P3 and so forth). The two protractors (P1 and P2) may be identical, or the two protractors (P1 and P2) may be non-identical. Where the peptide derivative comprises two or three protractors (P1, P2, P3), the protractors are preferably similar, more preferably substantially identical, or, most preferably, identical.

In the context of chemical moieties such as the protraction moieties disclosed herein, similarity and/or identity may be determined using any suitable computer program and/or algorithm known in the art.

The protraction moiety may be capable of non-covalently binding to albumin, thereby promoting the circulation of the peptide derivative of the present invention in the blood stream and prolonging its plasma half-life. Thus, the skilled person may also refer to the protraction moiety as being an “albumin binding moiety”.

### Protractor P

The protractor P may comprise an acyl group. The acyl group may be branched or unbranched. The acyl group may be saturated or unsaturated. The protractor P may comprise a fatty acid acyl group. The fatty acid acyl group may be branched or unbranched. The fatty acid acyl group may be saturated or unsaturated.

The protractor P may comprise a distal carboxylic acid group.

The protractor P may comprise a fatty acid group.

The protractor P may comprise a fatty acid group and an amide group.

The protractor P may comprise a distal carboxylic acid group and an amide group.

The protractor P may comprise an alkyl group.

The protractor P may comprise an aryl group.

The protractor P may comprise a tetrazole group.

The protractor P may comprise a sulfonic acid group.

The protractor P may comprise a phenoxy group.

The protractor P may comprise a benzoic acid group.

The protractor P may comprise a phosphonic acid group.

The protractor may comprise a group defined by:

Chem. 1a: HOOC—(CH₂)ₙ—CO—* wherein n is an integer in the range of 6-30, which may also be referred to as a C₍ₙ₊₂₎ diacid (e.g. C₁₈ diacid) or as

Chem. 1b:

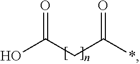

wherein n is an integer in the range of 6-30. The asterisk (*) shows the point of attachment of the radical.

The protractor P may comprise 8-32 carbon atoms. The protractor may comprise 8, 9, 10, 11, 12, 13, 14, 15, 16, 17, 18, 19, 20, 21, 22, 23, 24, 25, 26, 27, 28, 29, 30, 31 or 32 carbon atoms.

The protractor P may comprise 6-30 consecutive —CH₂— groups. The protractor P may comprise a carbon chain comprising at least 6, 7, 8, 9, 10, 11, 12, 13, 14, 15, 16, 17, 18, 19, 20, 21, 22, 23, 24, 25, 26, 27, 28, 29 or 30 consecutive —CH₂— groups.

The protractor P may comprise 12-26 carbon atoms. The protractor P may comprise 12, 13, 14, 15, 16, 17, 18, 19, 20, 21, 22, 23, 24, 25 or 26 carbon atoms.

The protractor P may comprise 10-26 consecutive —CH₂— groups. The protractor P may comprise a carbon chain comprising 10, 11, 12, 13, 14, 15, 16, 17, 18, 19, 20, 21, 22, 23, 24, 25 or 26 consecutive —CH₂— groups.

The protractor P may comprise 16-22 carbon atoms. The peptide derivative of the present invention may comprise a single protraction moiety with a protractor P comprising a carbon chain a side chain comprising 16, 17, 18, 19, 20, 21 or 22 carbon atoms.

The protractor P may comprise 14-20 consecutive —CH₂— groups. The protractor P may comprise a carbon chain comprising 14, 15, 16, 17, 18, 19 or 20 consecutive —CH₂— groups.

The protractor may P comprise 16-22 consecutive carbon atoms and 14-20 consecutive —CH₂— groups.

The protractor P may comprise 16 consecutive carbon atoms and 14 consecutive —CH₂— groups. The protractor P may be a C₁₆ diacid, which may be defined by the formula HOOC—(CH₂)₁₄—CO—*.

The protractor P may comprise 18 consecutive carbon atoms and 16 consecutive —CH₂— groups. The protractor P may be a C₁₈ diacid, which may be defined by the formula HOOC—(CH₂)₁₆—CO—*.

The protractor P may comprise 20 consecutive carbon atoms and 18 consecutive —CH₂— groups. The protractor P may be a C₂₀ diacid, which may be defined by the formula HOOC—(CH₂)₁₈—CO—*.

The protractor P may comprise 22 consecutive carbon atoms and 20 consecutive —CH₂— groups. The protractor P may be a C₂₂ diacid, which may be defined by the formula HOOC—(CH₂)₂₀—CO—*.

The term “fatty acid” refers to aliphatic mono- or dicarboxylic acids having from 4 to 28 carbon atoms, it may be branched or un-branched, it is preferably un-branched, and it may be saturated or unsaturated, it is preferably saturated.

As described above, the peptide derivative disclosed herein comprise one lysine (Lys, K) residue and hence one protraction moiety (L_{P}-P), wherein the protraction moiety is attached to the peptide backbone described herein via the epsilon position (i.e., amino group) of the lysine (Lys, K) residue (via an amide bond formed between a carboxylic acid group in the protraction moiety and the epsilon amino group of the lysine residue). The protraction moiety may be attached to the epsilon position of the one lysine (Lys, K) residue in the peptide backbone.

In one embodiment the protraction moiety may attach to the epsilon position of the lysine (Lys, K) residue in the peptide Z1 of the peptide backbone (the “Z1” in Z1-L1-Z2). In particular, the protraction moiety may attach to the epsilon position of the lysine (Lys, K) at position X₄ or X₅ of Formula IIa (SEQ ID NO: 3) of peptide Z1. In particular, the protraction moiety may attach to the epsilon position of the lysine (Lys, K) at position X₅₂ of Formula Xa (SEQ ID NO: 162) of peptide Z1. In particular, the protraction moiety may attach to the epsilon position of the lysine (Lys, K) at any one of positions 12, 33 or 34 of peptide Z1, preferably at position 12 or position 33 of peptide Z1.

In one embodiment the protraction moiety may attach to the epsilon position of the lysine (Lys, K) residue in the linker L1 portion of the peptide backbone (the “L1” in Z1-L1-Z2).

In one embodiment the protraction moiety may attach to the epsilon position of the lysine (Lys, K) residue in the peptide Z2 of the peptide backbone (the “22” in Z1-L1-Z2). In particular, the protraction moiety may attach to the epsilon position of the lysine (Lys, K) at any one of positions X₆, X₁₀, X₁₁, X₁₂, or X₁₄ of Formula IIIa (SEQ ID NO: 4) of peptide Z2. In particular, the protraction moiety may attach to the epsilon position of the lysine (Lys, K) at position X₆₃ of Formula XIIa (SEQ ID NO: 165) of peptide Z2. In particular, the protraction moiety may attach to the epsilon position of the lysine (Lys, K) residue at any one of positions 3, 15, 18, 20 or 24 of peptide Z2. In a preferred embodiment the protraction moiety may attach to the epsilon position of the lysine (Lys, K) residue at position 15 of peptide Z2 or at position 18 of peptide Z2.

In some embodiments the GLP-1-/GIP-/amylin-receptor tri-agonist comprising a peptide derivative as disclosed herein may comprise a protractor P, which is selected from any one of those depicted in

Table 3. R1 represents the site of attachment to (a) the backbone of the peptide derivative—more specifically the epsilon amino group of the lysine or (b) the present optional linker L_{P}. Based on the disclosure herein, the skilled person will be able to determine also other chemical moieties for use as a protractor in a specific peptide derivative as disclosed herein, optionally after some limited routine experiments.

TABLE 3
Examples of protractors (P)

| Protractor ID | Structure |
|---|---|
| Chem. 2 | 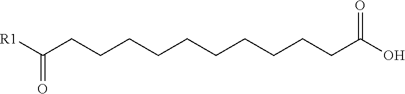 |
| | C₁₂ diacid |
| Chem. 3 | 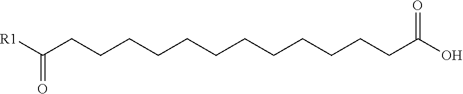 |
| | C₁₄ diacid |
| Chem. 4 | 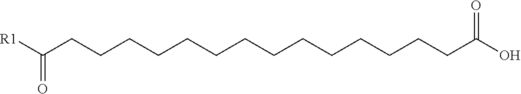 |
| | C₁₆ diacid |
| Chem. 5 | 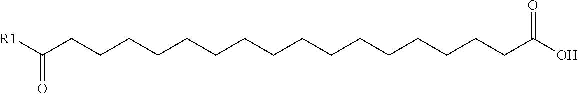 |
| | C₁₈ diacid |
| Chem. 6 | 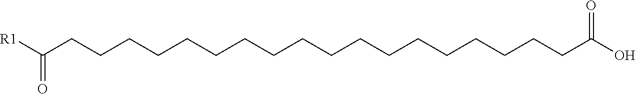 |
| | C₂₀ diacid |
| Chem. 7 | 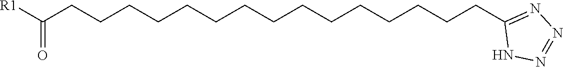 |
| | C₁₇ tetrazole |
| Chem. 8 | 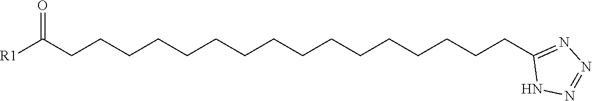 |
| | C₁₈ tetrazole |
| Chem. 32 | 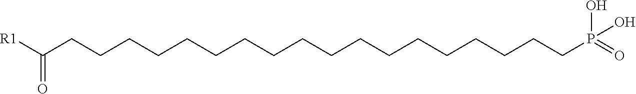 |
| | C₁₉ phosphonic acid |

In one embodiment the GLP-1-/GIP-/amylin-receptor tri-agonist comprises a peptide derivative comprising a protractor P, being a C₁₂-C₂₀ diacid.

In one embodiment the GLP-1-/GIP-/amylin-receptor tri-agonist comprises a peptide derivative comprising a protractor P selected from the group consisting of C₁₆ diacid, C₁₈ diacid, and C₂₀ diacid.

In a preferred embodiment the GLP-1-/GIP-/amylin-receptor tri-agonist comprises a peptide derivative comprising a protractor P being a C₁₈ diacid or a C₂₀ diacid.

### Linker L_{P}

In one embodiment the protractor is attached/conjugated directly onto the backbone of the peptide derivative, i.e., without use of a linker L_{P} (i.e., by way of a covalent bond, e.g. an amide bond).

In other embodiments the protractor is covalently conjugated to the peptide derivative using a linker L_{P}, hence, as described above, the protraction moiety (L_{P}-P) comprises an optional linker L_{P}. The linker L_{P} may comprise several “linker elements”. The linker elements may be selected so that they improve the overall properties of the molecule, e.g., so that they improve the oral bioavailability, the conversion of half-life or the protracting effect, thus improving the overall exposure profile upon oral administration of the compound.

The linker L_{P} may comprise Ado, Aeep or Aeeep, Ahx, Ala, ε-Lys, Glu, γGlu, Gly, Ser, sulfonamide, Thr and/or Trx.

The linker L_{P} may comprise at least a moiety which may be represented by the following chemical formula (wherein the asterisks (*) show the points of attachment of the radicals):

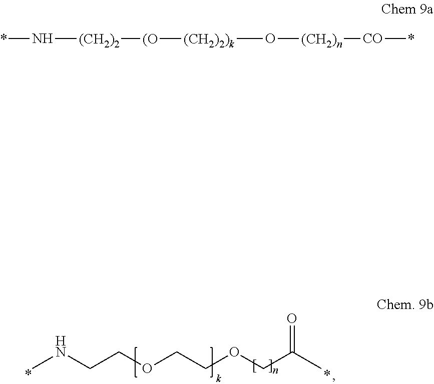

wherein k is an integer in the range of 1-5, and n is an integer in the range of 1-5.

When k=1 and n=1, the linker element may be designated Ado, or 8-amino-3,6-dioxaoctanoyl, which may be represented by the following chemical formula:

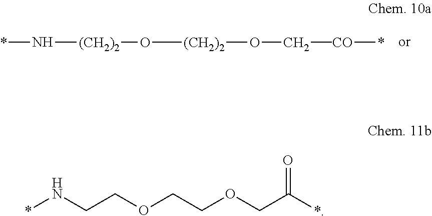

When k=1 and n=2, the linker element may be designated Aeep, which may be represented by the following chemical formula:

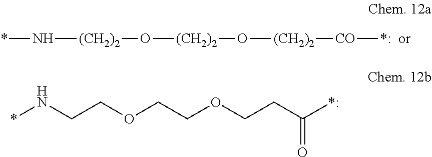

When k=2 and n=2, the linker element may be designated Aeeep, which may be represented by the following chemical formula:

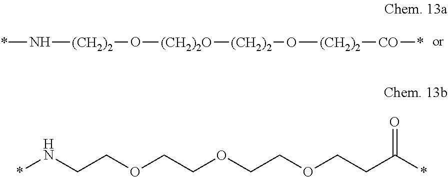

The linker L_{P} may comprise a sulfonamide-C4 moiety. A sulfonamide-C4 group is a sulfonamide group attached to a 4-butanoyl group, having the following chemical formula:

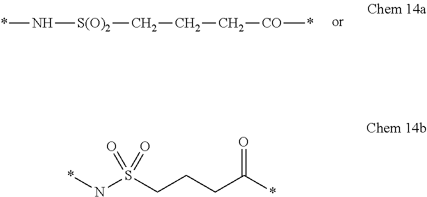

The linker L_{P} may comprise Trx. Trx is also referred to as Tranexamic acid, trans-4-(aminomethyl)cyclohexanecarboxylic acid, having the following chemical formula:

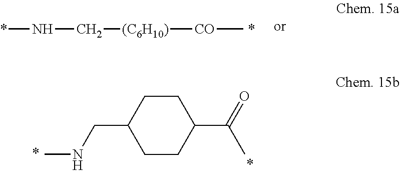

The linker L_{P} may comprise Ahx. Ahx is also referred to as Aminocaproic acid, 6-aminohexanoic acid having the following chemical formula:

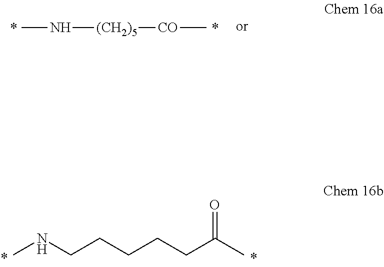

The linker L_{P} may comprise epsilon-lysine (c-Lys).

The linker L_{P} may comprise lysine (Lys).

The linker L_{P} may comprise alanine (Ala).

The linker L_{P} may comprise glycine (Gly).

The linker L_{P} may comprise serine (Ser).

The linker L_{P} may comprise glutamic acid (Glu).

The linker L_{P} may comprise a Glu di-radical, such as

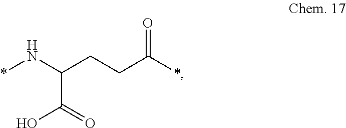

wherein the Glu di-radical may be included p times, where p is an integer in the range of 1-3. Anyone of above disclosed amino acids, which are used as linker L_{P} or as part of linker L_{P}, may be used as L-isomer or as D-isomer.

Chem. 17 may also be referred to as gamma-Glu, or briefly γGlu, due to the fact that it is the gamma carboxy group of the amino acid glutamic acid which is here used for connection to the epsilon amino group of lysine. As described above, the other linker element may, for example, be another Glu residue, or an Ado molecule. The amino group of Glu in turn forms an amide bond with the carboxy group of the protracting moiety, or with the carboxy group of, e.g., an Ado molecule, if present, or with the gamma-carboxy group of, e.g., another Glu, if present.

The peptide derivative disclosed herein may comprise a linker L_{P} which is selected from any one of those depicted in Table 4 below. R1 represents the residue in the peptide backbone to which the protraction moiety is attached to, and P represents the protractor.

In some embodiments the peptide derivative comprises a protraction moiety, wherein the protractor Chem. 4 or Chem. 5 or Chem. 6 is attached to the peptide backbone using the linker designated L_{P}1, L_{P}2, L_{P}3, L_{P}4, L_{P}5 or L_{P}6 in Table 4 below.

In some embodiments the peptide derivative comprises a protraction moiety, wherein the protractor Chem. 5 is attached to the peptide backbone using the linker designated L_{P}1, L_{P}2, L_{P}3 L_{P}4, L_{P}5 or L_{P}6 in Table 4 below, thus wherein said protraction moiety comprises Chem. 18, Chem. 19, Chem. 20, Chem. 21, Chem. 33 or Chem. 34 as linker L_{P} and Chem. 5 as protractor P.

In some embodiments the peptide derivative comprises a protraction moiety, wherein the protractor Chem. 6 is attached to the peptide backbone using the linker designated L_{P}1, L_{P}2, L_{P}3 L_{P}4, L_{P}5 or L_{P}6 in Table 4 below, thus wherein said protraction moiety comprises Chem. 18, Chem. 19, Chem. 20, Chem. 21, Chem. 33 or Chem. 34 as linker L_{P} and Chem. 6 as protractor P.

In a preferred embodiment the peptide derivative comprises a protraction moiety, wherein said protraction moiety comprises Chem. 20 or Chem. 21 as linker L_{P} and Chem. 5 or Chem. 6 as protractor P.

In a preferred embodiment the peptide derivative comprises a protraction moiety, wherein the protractor Chem. 5 is attached to the peptide backbone using the linker designated L_{P}3 in Table 4 below, thus wherein said protraction moiety comprises Chem. 20 as linker L_{P} and Chem. 5 as protractor P.

In a preferred embodiment the peptide derivative comprises a protraction moiety, wherein the protractor Chem. 6 is attached to the peptide backbone using the linker designated L_{P}3 in Table 4 below, thus wherein said protraction moiety comprises Chem. 20 as linker L_{P} and Chem. 6 as protractor P.

Based on the disclosure herein, the skilled person will be able to determine the optimal L_{P} linker for use in a specific peptide derivative as disclosed herein, optionally after some limited routine experiments.

TABLE 4
Examples of optional linkers (Lₚ) of the protraction moiety

| Linker Lₚ | |
|---|---|
| ID | Structure |
| Lₚ1 (Chem. 18) | 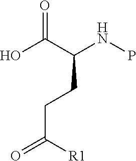 |
| Lₚ2 (Chem. 19) | 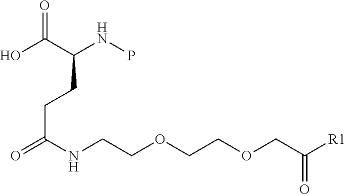 |
| Lₚ3 (Chem. 20) | 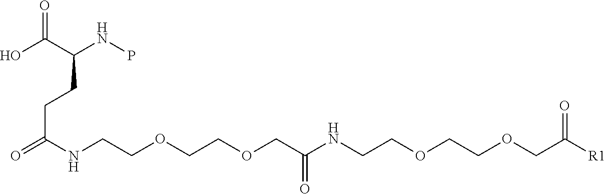 |
| Lₚ4 (Chem. 21) | 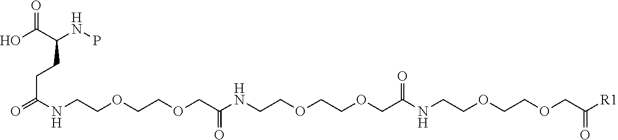 |
| Lₚ5 (Chem. 33) | 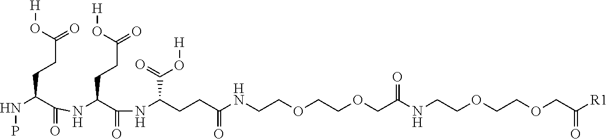 |
| Lₚ6 (Chem. 34) | 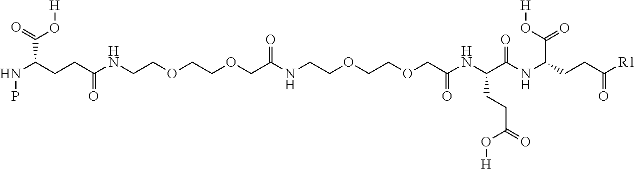 |

In some embodiments the peptide derivative comprises a protraction moiety which is selected from the group presented in Table 5. R1 represents the residue in the peptide backbone to which the protraction moiety is attached to.

TABLE 5
Examples of protraction moieties

| Protraction moiety | Structure |
|---|---|
| C₂₀ diacid gamma-Glu (Chem. 22) | 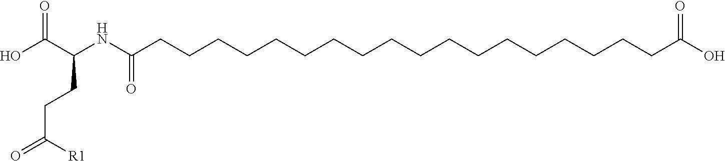 |
| C₂₀ diacid gamma-Glu gamma-Glu (Chem. 23) | 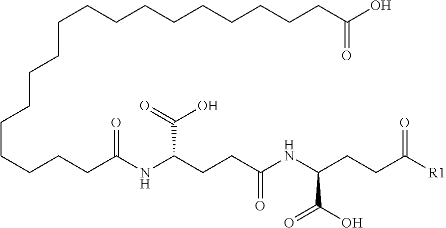 |
| C₂₀ diacid gamma-Glu gamma-Glu gamma-Glu (Chem. 24) | 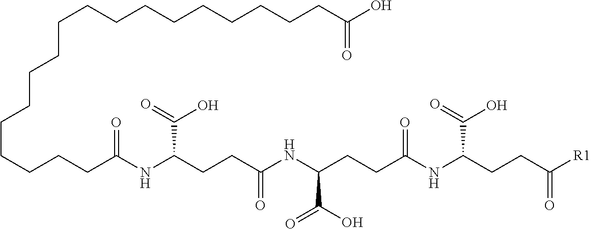 |
| C₂₀ diacid Ado (Chem. 25) | 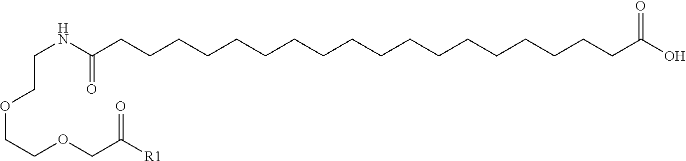 |
| C₂₀ diacid gamma-Glu Ado (Chem. 26) | 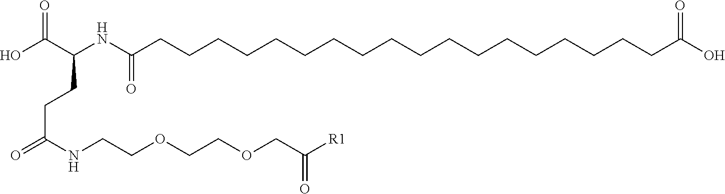 |
| C₂₀ diacid gamma-Glu 2xAdo (Chem. 27) | 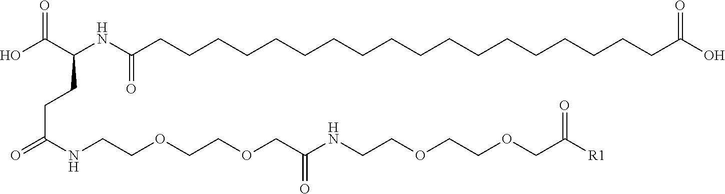 |
| C₁₈ diacid gamma-Glu 2xAdo (Chem. 28) | 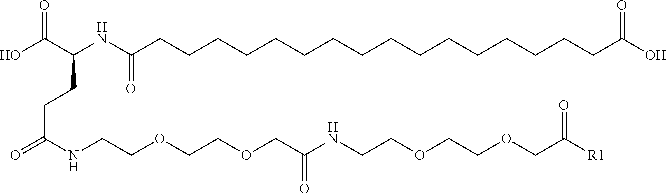 |
| C₁₆ diacid gamma-Glu 2xAdo (Chem. 29) | 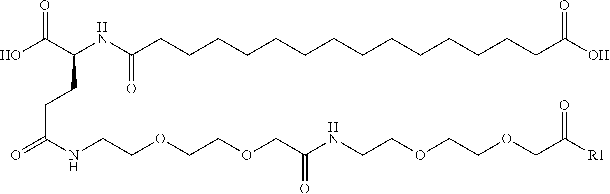 |
| C₁₈ diacid gamma-Glu (Chem. 30) | 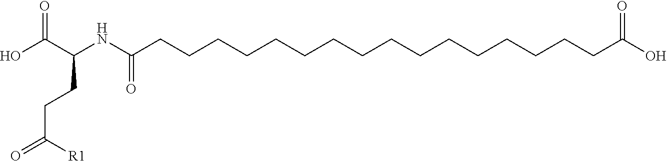 |
| C₁₈ diacid gamma-Glu 3xAdo (Chem. 31) | 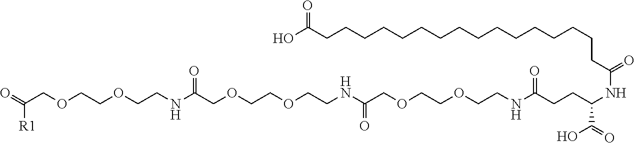 |
| C₁₉ phosphonic acid gamma-Glu 2xAdo (Chem. 35) | 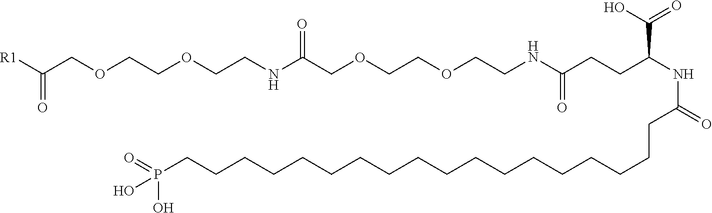 |
| (Chem. 36) C₂₀ diacid 2×Glu gamma-Glu 2xAdo | 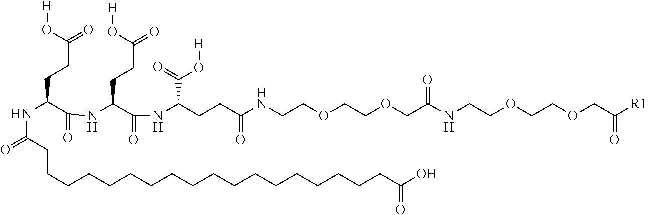 |
| (Chem. 37) C₂₀ diacid gamma-Glu 2xAdo Glu gamma-Glu | 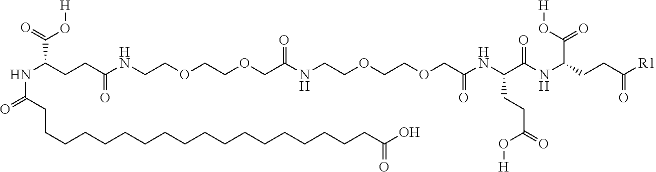 |

In one embodiment the free lysine may serve as a conjugation site for attaching one C₁₆ diacid gamma-Glu 2×Ado fatty acid moiety (IUPAC name [2-[2-[2-[[2-[2-[2-[[(4S)-4-carboxy-4-(15-carboxypentadecanoylamino)butanoyl]amino]ethoxy]ethoxy]acetyl]amino]ethoxy]ethoxy]acetyl]), and thus the peptide derivative of the present invention comprises a protraction moiety, wherein said protraction moiety is a C₁₆ diacid (S) gamma-Glu 2×Ado fatty acid moiety.

In one embodiment a free lysine may serve as a conjugation site for attaching one C₁₈ diacid gamma-Glu 2×Ado fatty acid moiety (IUPAC name [2-[2-[2-[[2-[2-[2-[[(4S)-4-carboxy-4-(17-carboxyheptadecanoylamino)butanoyl]amino]ethoxy]ethoxy]acetyl]amino]ethoxy]ethoxy]acetyl]), and thus the peptide derivative of the present invention comprises a protraction moiety, wherein said protraction moiety is a C₁₈ diacid (S) gamma-Glu 2×Ado fatty acid moiety.

In one embodiment a free lysine may serve as a conjugation site for attaching one C₂₀ diacid gamma-Glu 2×Ado fatty acid moiety (IUPAC name [2-[2-[2-[[2-[2-[2-[[(4S)-4-carboxy-4-(19-carboxynonadecanoylamino)butanoyl]amino]ethoxy]ethoxy]acetyl]amino]ethoxy]ethoxy]acetyl]), and thus the peptide derivative of the present invention comprises a protraction moiety, wherein said protraction moiety is a C₂₀ diacid (S) gamma-Glu 2×Ado fatty acid moiety.

In one embodiment a free lysine may serve as a conjugation site for attaching one C₁₈ diacid gamma-Glu fatty acid moiety (IUPAC name [(4S)-4-carboxy-4-(17-carboxy-heptadecanoylamino)butanoyl]), and thus the peptide derivative of the present invention comprises a protraction moiety, wherein said protraction moiety is a C₁₈ diacid (S) gamma-Glu fatty acid moiety.

In one embodiment a free lysine may serve as a conjugation site for attaching one C₂₀ diacid gamma-Glu fatty acid moiety (IUPAC name [(4S)-4-carboxy-4-(19-carboxy-nonadecanoylamino)butanoyl]), and thus the peptide derivative of the present invention comprises a protraction moiety, wherein said protraction moiety is a C₂₀ diacid (S) gamma-Glu fatty acid moiety.

In a preferred embodiment a free lysine may serve as a conjugation site for attaching one C₁₈ diacid gamma-Glu 2×Ado fatty acid moiety (IUPAC name [2-[2-[2-[[2-[2-[2-[[(4S)-4-carboxy-4-(17-carboxypentadecanoylamino)butanoyl]amino]ethoxy]ethoxy]acetyl]amino]ethoxy]ethoxy]acetyl]), and thus the peptide derivative of the present invention comprises a protraction moiety, wherein said protraction moiety is a C₁₈ diacid (S) gamma-Glu 2×Ado fatty acid moiety.

In a preferred embodiment a free lysine may serve as a conjugation site for attaching one C₂₀ diacid gamma-Glu 2×Ado fatty acid moiety (IUPAC name [2-[2-[2-[[2-[2-[2-[[(4S)-4-carboxy-4-(19-carboxynonadecanoylamino)butanoyl]amino]ethoxy]ethoxy]acetyl]amino]ethoxy]ethoxy]acetyl]), and thus the peptide derivative of the present invention comprises a protraction moiety, wherein said protraction moiety is a C₂₀ diacid (S) gamma-Glu 2×Ado fatty acid moiety.

In a most preferred embodiment a free lysine may serve as a conjugation site for attaching one C₁₈ diacid gamma-Glu 2×Ado fatty acid moiety (IUPAC name [2-[2-[2-[[2-[2-[2-[[(4S)-4-carboxy-4-(17-carboxyheptadecanoylamino)butanoyl]amino]ethoxy]ethoxy]acetyl]amino]ethoxy]ethoxy]acetyl]), and thus the peptide derivative of the present invention comprises a protraction moiety, wherein said protraction moiety is a C₁₈ diacid (S) gamma-Glu 2×Ado fatty acid moiety.

### Peptide Derivative

As described above, the GLP-1-/GIP-/amylin-receptor tri-agonist of the present invention may comprise a peptide linker and further comprise a protraction moiety. Therefore, the present invention relates in another aspect to a GLP-1-/GIP-/amylin-receptor tri-agonist comprising a peptide according to Formula I:

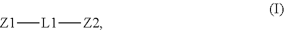

comprising one lysine (Lys, K) residue; wherein:
Z1 is a peptide having a maximum of 4 amino acid substitutions relative to Formula II (SEQ ID NO: 1):

(II)
YX₂EGTFTSDYSILLEEQAAREFIEWLLAGGPSKG,

wherein the amino acid at position X₂ represents Aib, and
Z1 comprises or consists of an amino acid sequence according to Formula X (SEQ ID NO: 161):

(X)
X₂₁X₂₂X₂₃GTFTSDYSX₂₄LLEEX₂₅AAX₂₆EFIX₂₇WLX₂₈X₂₉GGPSX₃₀X₃₁,

wherein
X₂₁ represents His (H) or Tyr (Y),
X₂₂ represents Aib,
X₂₃ represents Glu (E) or His (H),
X₂₄ represents Ile (I) or Lys (K),
X₂₅ represents Gln (Q) or Ile (I),
X₂₆ represents Arg (R) or Gln (Q),
X₂₇ represents Ala (A), Glu (E) or Gln (Q),
X₂₈ represents Leu (L) or I (Ile),
X₂₉ represents Ala (A) or Gln (Q),
X₃₀ represents Arg (R), Gly (G), Lys (K) or Ser (S),
X₃₁ represents Gly (G), Glu (E) or Lys (K);
L1 is a peptide linker;
Z2 is a peptide comprising a C-terminal amide, and having a maximum of 10 amino acid substitutions relative to Formula III (SEQ ID NO: 2):

(III)
ASELSTAALGRLSAELHELATLPRTETGSGSP,

and
Z2 comprises or consists of an amino acid sequence according to Formula XII (SEQ ID NO:164):

(XII)
AX₃₂X₃₃LSTAX₃₄X₃₅X₃₆RLSAX₃₇LHX₃₈LX₃₉X₄₀X₄₁PX₄₂TETGSGX₄₃P,

wherein
X₃₂ represents Gly (G) or Ser (S),
X₃₃ represents Gln (Q), Glu (E), His (H) or Lys (K),
X₃₄ represents Ala (A) or Gln (Q),
X₃₅ represents Gln (Q), Leu (L) or Thr (T),
X₃₆ represents Ala (A), Gly (G) or Gln (Q),
X₃₇ represents Glu (E) or Lys (K),
X₃₈ represents Arg (R), Gln (Q), Glu (E), Gly (G), His (H), Lys (K), Thr (T) or Tyr (Y),
X₃₉ represents Ala (A) or Lys (K),
X₄₀ represents Asp (D) or Thr (T),
X₄₁ represents Leu (L) or Glu (E),
X₄₂ represents Arg (R) or Lys (K),
X₄₃ represents Ala (A) or Ser (S); and wherein the peptide is a peptide derivative comprising a protraction moiety.

In another aspect the present invention relates to a GLP-1-/GIP-/amylin-receptor tri-agonist comprising a peptide according to Formula I:

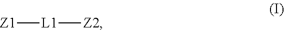

comprising one lysine (Lys, K) residue; wherein:
Z1 is a peptide having a maximum of 4 amino acid substitutions relative to Formula II (SEQ ID NO: 1):

(II)
YX₂EGTFTSDYSILLEEQAAREFIEWLLAGGPSKG,

wherein the amino acid at position X₂ represents Aib, and
Z1 comprises or consists of an amino acid sequence according to Formula IIa (SEQ ID NO: 3):

(IIa)
X₁X₂X₃GTFTSDYSILLEEQAAREFIEWLLAGGPSX₄X₅,

wherein
X₁ represents His (H) or Tyr (Y),
X₂ represents Aib,
X₃ represents Glu (E) or His (H),
X₄ represents Arg (R), Gly (G), Lys (K) or Ser (S),
X₅ represents Gly (G) or Lys (K);
L1 is a peptide linker;
Z2 is a peptide comprising a C-terminal amide, and having a maximum of 10 amino acid substitutions relative to Formula III (SEQ ID NO: 2):

(III)
ASELSTAALGRLSAELHELATLPRTETGSGSP,

and
Z2 comprises or consists of an amino acid sequence according to Formula IIIa (SEQ ID NO: 4):

(IIa)
ASX₆LSTAX₇X₈X₉RLSAX₁₀LHX₁₁LX₁₂X₁₃LPX₁₄TETGSGX₁₅P,

wherein
X₆ represents Gln (Q), Glu (E), His (H) or Lys (K),
X₇ represents Ala (A) or Gln (Q),
X₈ represents Gln (Q), Leu (L) or Thr (T),
X₉ represents Ala (A), Gly (G) or Gln (Q),
X₁₀ represents Glu (E) or Lys (K),
X₁₁ represents Arg (R), Gln (Q), Glu (E), Gly (G), His (H), Lys (K), Thr (T) or Tyr (Y),
X₁₂ represents Ala (A) or Lys (K),
X₁₃ represents Asp (D) or Thr (T),
X₁₄ represents Arg (R) or Lys (K),
X₁₅ represents Ala (A) or Ser (S); and
   wherein the peptide is a peptide derivative comprising a protraction moiety.

In another aspect the invention relates to a GLP-1-/GIP-/amylin-receptor tri-agonist comprising a peptide according to Formula I:

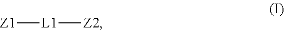

comprising one lysine (Lys, K) residue; wherein:
Z1 is a peptide having a maximum of 4 amino acid substitutions relative to Formula II (SEQ ID NO: 1):

(II)
YX₂EGTFTSDYSILLEEQAAREFIEWLLAGGPSKG,

wherein the amino acid at position X₂ represents Aib, and
Z1 comprises or consists of an amino acid sequence according to Formula Xa (SEQ ID NO: 162):

(Xa)
YX₅₁EGTFTSDYSX₅₂LLEEIAAX₅₃EFIX₅₄WLX₅₅X₅₆GGPSSX₅₇,

wherein
X₅₁ represents Aib,
X₅₂ represents Ile (I) or Lys (K),
X₅₃ represents Arg (R) or Gln (Q),
X₅₄ represents Ala (A), Glu (E) or Gln (Q),
X₅₅ represents Leu (L) or I (Ile),
X₅₆ represents Ala (A) or Gln (Q),
X₅₇ represents Gly (G) or Glu (E);
L1 is a peptide linker;
Z2 is a peptide comprising a C-terminal amide, and having a maximum of 10 amino acid substitutions relative to Formula III (SEQ ID NO: 2):

(III)
ASELSTAALGRLSAELHELATLPRTETGSGSP,

and
Z2 comprises or consists of an amino acid sequence according to Formula XIIa (SEQ ID NO: 165):

(XIIa)
AX₅₈X₅₉LSTAX₆₀X₆₁X₆₂RLSAELHX₆₃LATX₆₄PRTETGSGSP,

wherein
   X₅₈ represents Gly (G) or Ser (S),
   X₅₉ represents Gln (Q), Glu (E), or His (H),
   X₆₀ represents Ala (A) or Gln (Q),
   X₆₁ represents Leu (L) or Thr (T),
   X₆₂ represents Ala (A), Gly (G) or Gln (Q),
   X₆₃ represents Gln (Q), Glu (E), or Lys (K),
   X₅₄ represents Leu (L) or Glu (E); and
      wherein the peptide is a peptide derivative comprising a protraction moiety.

In yet another aspect the present invention relates to a GLP-1-/GIP-/amylin-receptor tri-agonist comprising a peptide according to Formula I:

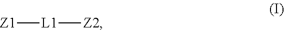

comprising one lysine (Lys, K) residue; wherein:
Z1 is a peptide having a maximum of 4 amino acid substitutions relative to Formula II (SEQ ID NO: 1):

(II)
YX₂EGTFTSDYSILLEEQAAREFIEWLLAGGPSKG,

wherein the amino acid at position X₂ represents Aib,
and
Z1 comprises or consists of an amino acid sequence according to Formula X (SEQ ID NO: 161):

(X)
X₂₁X₂₂X₂₃GTFTSDYSX₂₄LLEEX₂₅AAX₂₆EFIX₂₇WLX₂₈X₂₉GGPSX₃₀X₃₁,

wherein
X₂₁ represents His (H) or Tyr (Y),
X₂₂ represents Aib,
X₂₃ represents Glu (E) or His (H),
X₂₄ represents Ile (I) or Lys (K),
X₂₅ represents Gln (Q) or Ile (I),
X₂₆ represents Arg (R) or Gln (Q),
X₂₇ represents Ala (A), Glu (E) or Gln (Q),
X₂₈ represents Leu (L) or I (Ile),
X₂₉ represents Ala (A) or Gln (Q),
X₃₀ represents Arg (R), Gly (G), Lys (K) or Ser (S),
X₃₁ represents Gly (G), Glu (E) or Lys (K);
L1 is a peptide linker comprising or consisting of the amino acid sequence according to Formula IV:

(IV)
X₁X₂X₃X₄X₅X₆X₇X₈X₉X₁₀X₁₁X₁₂X₁₃X₁₄,

which is selected from the group consisting of E, GE, APPPSGGGE (SEQ ID NO: 129), AGQAPGQAPG (SEQ ID NO: 136), AGQAPGQAPL (SEQ ID NO: 137) and AGQAPGEAPG (SEQ ID NO: 154);
Z2 is a peptide comprising a C-terminal amide, and having a maximum of 10 amino acid substitutions relative to Formula III (SEQ ID NO: 2):

(III)
ASELSTAALGRLSAELHELATLPRTETGSGSP,

and
Z2 comprises or consists of an amino acid sequence according to Formula XII (SEQ ID NO:164):

(XII)
AX₃₂X₃₃LSTAX₃₄X₃₅X₃₆RLSAX₃₇LHX₃₈LX₃₉X₄₀X₄₁PX₄₂TETGSGX₄₃P,

wherein
X₃₂ represents Gly (G) or Ser (S),
X₃₃ represents Gln (Q), Glu (E), His (H) or Lys (K),
X₃₄ represents Ala (A) or Gln (Q),
X₃₅ represents Gln (Q), Leu (L) or Thr (T),
X₃₆ represents Ala (A), Gly (G) or Gln (Q),
X₃₇ represents Glu (E) or Lys (K),
X₃₈ represents Arg (R), Gln (Q), Glu (E), Gly (G), His (H), Lys (K), Thr (T) or Tyr (Y),
X₃₉ represents Ala (A) or Lys (K),
X₄₀ represents Asp (D) or Thr (T),
X₄₁ represents Leu (L) or Glu (E),
X₄₂ represents Arg (R) or Lys (K),
X₄₃ represents Ala (A) or Ser (S); and
   wherein the peptide is a peptide derivative comprising a protraction moiety.

In yet another aspect the present invention relates to a GLP-1-/GIP-/amylin-receptor tri-agonist comprising a peptide according to Formula I:

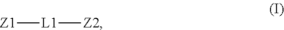

comprising one lysine (Lys, K) residue; wherein:
Z1 is a peptide having a maximum of 4 amino acid substitutions relative to Formula II (SEQ ID NO: 1):

(II)
YX₂EGTFTSDYSILLEEQAAREFIEWLLAGGPSKG,

wherein the amino acid at position X₂ represents Aib, and
Z1 comprises or consists of an amino acid sequence according to Formula IIa (SEQ ID NO: 3):

(IIa)
X₁X₂X₃GTFTSDYSILLEEQAAREFIEWLLAGGPSX₄X₅,

wherein
X₁ represents His (H) or Tyr (Y),
X₂ represents Aib,
X₃ represents Glu (E) or His (H),
X₄ represents Arg (R), Gly (G), Lys (K) or Ser (S),
X₅ represents Gly (G) or Lys (K);
L1 is a peptide linker comprising or consisting of the amino acid sequence according to Formula IV:

(IV)
X₁X₂X₃X₄X₅X₆X₇X₈X₉X₁₀X₁₁X₁₂X₁₃X₁₄,

which is selected from the group consisting of E, GE, APPPSGGGE (SEQ ID NO: 129), AGQAPGQAPG (SEQ ID NO: 136), AGQAPGQAPL (SEQ ID NO: 137) and AGQAPGEAPG (SEQ ID NO: 154);
Z2 is a peptide comprising a C-terminal amide and having a maximum of 10 amino acid substitutions relative to Formula III (SEQ ID NO: 2):

(III)
ASELSTAALGRLSAELHELATLPRTETGSGSP,

and
Z2 comprises or consists of an amino acid sequence according to Formula IIIa (SEQ ID NO: 4):

(IIIa)
ASX₆LSTAX₇X₈X₉RLSAX₁₀LHX₁₁LX₁₂X₁₃LPX₁₄TETGSGX₁₅P,

wherein
X₆ represents Gln (Q), Glu (E), His (H) or Lys (K),
X₇ represents Ala (A) or Gln (Q),
X₈ represents Gln (Q), Leu (L) or Thr (T),
X₉ represents Ala (A), Gly (G) or Gln (Q),
X₁₀ represents Glu (E) or Lys (K),
X₁₁ represents Arg (R), Gln (Q), Glu (E), Gly (G), His (H), Lys (K), Thr (T) or Tyr (Y),
X₁₂ represents Ala (A) or Lys (K),
X₁₃ represents Asp (D) or Thr (T),
X₁₄ represents Arg (R) or Lys (K),
X₁₅ represents Ala (A) or Ser (S); and
wherein the peptide is a peptide derivative comprising a protraction moiety.

In yet another aspect the invention relates to a GLP-1-/GIP-/amylin-receptor tri-agonist comprising a peptide according to Formula I:

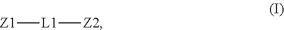

comprising one lysine (Lys, K) residue; wherein:
Z1 is a peptide having a maximum of 4 amino acid substitutions relative to Formula II (SEQ ID NO: 1):

(II)
YX₂EGTFTSDYSILLEEQAAREFIEWLLAGGPSKG,

wherein the amino acid at position X₂ represents Aib,
and
Z1 comprises or consists of an amino acid sequence according to Formula Xa (SEQ ID NO: 162):

(Xa)
YX₅₁EGTFTSDYSX₅₂LLEEIAAX₅₃EFIX₅₄WLX₅₅X₅₆GGPSSX₅₇,

wherein
X₅₁ represents Aib,
X₅₂ represents Ile (I) or Lys (K),
X₅₃ represents Arg (R) or Gln (Q),
X₅₄ represents Ala (A), Glu (E) or Gln (Q),
X₅₅ represents Leu (L) or I (Ile),
X₅₆ represents Ala (A) or Gln (Q),
X₅₇ represents Gly (G) or Glu (E);
L1 is a peptide linker comprising or consisting of the amino acid sequence according to Formula IV:

(IV)
X₁X₂X₃X₄X₅X₆X₇X₈X₉X₁₀X₁₁X₁₂X₁₃X₁₄,

which is selected from the group consisting of E, GE, APPPSGGGE (SEQ ID NO: 129), AGQAPGQAPG (SEQ ID NO: 136), AGQAPGQAPL (SEQ ID NO: 137) and AGQAPGEAPG (SEQ ID NO: 154);
Z2 is a peptide comprising a C-terminal amide, and having a maximum of 10 amino acid substitutions relative to Formula III (SEQ ID NO: 2):

(III)
ASELSTAALGRLSAELHELATLPRTETGSGSP,

and

Z2 comprises or consists of an amino acid sequence according to Formula XIIa (SEQ ID NO: 165):

(XIIa)
AX₅₈X₅₉LSTAX₆₀X₆₁X₆₂RLSAELHX₆₃LATX₆₄PRTETGSGSP,

wherein
X₅₈ represents Gly (G) or Ser (S),
X₅₉ represents Gln (Q), Glu (E), or His (H),
X₆₀ represents Ala (A) or Gln (Q),
X₆₁ represents Leu (L) or Thr (T),
X₆₂ represents Ala (A), Gly (G) or Gln (Q),
X₆₃ represents Gln (Q), Glu (E), or Lys (K),
X₆₄ represents Leu (L) or Glu (E); and
   wherein the peptide is a peptide derivative comprising a protraction moiety.

The GLP-1-/GIP-/amylin-receptor tri-agonists of the present invention may be a peptide derivative which comprise any one of the above disclosed peptides Z1, any one of the above disclosed peptides Z2, any one of the above disclosed peptide linkers L1 and any one of the above disclosed protraction moieties, and based on the disclosure herein, the skilled person will be able to determine the optimal combination to come to a specific peptide derivative which is a potent GLP-1-/GIP-/amylin-receptor tri-agonist having specific properties as described below.

When tested as described in “GLP-1 receptor assay” (preferably method A; in the absence of HSA), the peptide derivative disclosed herein may have an EC₅₀ value of less than 125 pM, preferably less than 100 pM, such as less than 75 pM, even more preferably less than 50 pM, such as less than 40 pM, and most preferably less than 30 pM, such as less than 20 pM, such as less than 10 pM, such as less than 5 pM.

When tested as described in “GIP receptor assay” (preferably method A; in the absence of HSA), the peptide derivative disclosed herein may have an EC₅₀ value of less than 125 pM, preferably less than 100 pM, such as less than 75 pM, even more preferably less than 50 pM, such as less than 40 pM, and most preferably less than 30 pM, such as less than 20 pM, such as less than 10 pM, such as less than 5 pM.

When tested as described in “Amylin receptor assay” (preferably method A; in the absence of HSA), the peptide derivative disclosed herein may have an EC₅₀ value of less than 125 pM, preferably less than 100 pM, such as less than 75 pM, even more preferably less than 50 pM, such as less than 40 pM, and most preferably less than 30 pM, such as less than 20 pM, such as less than 10 pM, such as less than 5 pM.

The GLP-1-/GIP-/amylin-receptor tri-agonist according to any one of the preceding embodiments, which activates the human GIP, GLP-1 and amylin receptors in vitro, when measured without HSA in assays as described in Example 4, and which has a wherein potency ratio (A/B) of less than 50.

The balanced GLP-1-/GIP-/amylin-receptor tri-agonist of the present invention activates the human GIP, GLP-1, and amylin receptors in vitro, when measured without HSA in assays as described in Example 4, and has a potency ratio (A/B), i.e., potency (A) of the receptor with lowest potency divided by potency (B) of the receptor with highest potency, of less than 50. The tri-agonists disclosed herein may have a potency ratio (A/B) of less than 50, preferably less than 20, such as less than 19, less than 18, less than 17, less than 16, even more preferred less than 15, such as less than 14, less than 13, less than 12, and most preferred less than 11, such as less than 10, less than 9, less than 8 and less than 7. Half-life is an important parameter as a long half-life indicates that less frequent administration of a compound may be possible. Based on the disclosure herein, the skilled person will be able to determine the protraction moiety for use in a specific peptide derivative as disclosed herein, optionally after some limited routine experiments. Hence in a fourth aspect, the present invention relates to a GLP-1-/GIP-/amylin-receptor tri-agonist having improved pharmacokinetic properties. The GLP-1-/GIP-/amylin-receptor tri-agonists or the peptide derivatives of the present invention have a long half-life relative to dosing interval, thus reducing the variability in steady state exposure.

The in vivo pharmacology, including half-life, of the GLP-1-/GIP-/amylin-receptor tri-agonist described herein may be assessed as described in Example 6. In some embodiments, the half-life is half-life (t½) in vivo in minipigs after i.v. administration, e.g., as described in Example 6. The half-life of the GLP-1-/GIP-/amylin-receptor tri-agonist in animal subjects may be as long as about 100 hours, or longer. The half-life of the GLP-1-/GIP-/amylin-receptor tri-agonist in animal subjects may be at least 40 hours, preferably at least 100 hours. The half-life of the GLP-1-/GIP-/amylin-receptor tri-agonist may be more than 40, 45, 55, 60, 60, 65, 70, 75, 80, 85, 90, 95, 100, 105, 110, 115, 120, 125, 130, 135, or 140 hours. The half-life of the GLP-1-/GIP-/amylin-receptor tri-agonist may be 40-145 hours, such as 90-140 hours, such as 85-125 hours.

In a fifth aspect, the present invention relates to a GLP-1-/GIP-/amylin-receptor tri-agonist which is suitable for once weekly administration. The GLP-1-/GIP-/amylin-receptor tri-agonists or the peptide derivatives of the present invention have a long half-life relative to dosing interval, thus reducing the variability in steady state exposure, and thus making a once weekly administration possible.

In a sixth aspect, the present invention relates to a GLP-1-/GIP-/amylin-receptor tri-agonist which is suitable for oral administration. The GLP-1-/GIP-/amylin-receptor tri-agonist described herein may be orally bioavailable; that is, present in the bloodstream following per oral administration. Therefore, the compound is suitable for oral administration of subjects in need thereof.

In a seventh aspect, the present invention relates to a GLP-1-/GIP-/amylin-receptor tri-agonist having improved chemical stability. The term “chemical stability” refers to chemical (in particular covalent) changes in the polypeptide structure leading to formation of chemical degradation products, such as high molecular weight proteins (HMWPs), deamidation, isomerization and hydrolysis products potentially having a reduced biological potency, and/or increased immunogenic effect as compared to the intact polypeptide. The chemical stability may be determined by measuring the purity loss, e.g., by measuring the amount of chemical degradation products at various time-points after exposure to different environmental conditions, e.g., by SEC-HPLC, and/or LCMS, e.g., as described in Example 7 herein. The GLP-1-/GIP-/amylin-receptor tri-agonist of the invention has a purity loss per week of less than 10.0 percent, preferably less than 6.0 percent, such as 5.0 or 4.0 percent, more preferably less than 3.0 percent, and most preferred less than 1.5 percent, upon incubation at 37° C. and as determined in Example 7 described herein.

In one embodiment the GLP-1-/GIP-/amylin-receptor tri-agonist disclosed herein may reduce food intake in a subject, e.g., normal weight rats or DIO rats. Administration of the GLP-1-/GIP-/amylin-receptor tri-agonist disclosed herein may result in an acute reduction in the intake of food. The in vivo effect of the GLP-1-/GIP-/amylin-receptor tri-agonist on food intake in rats may be assessed as described in Example 5 or Example 8. A reduction of food intake of 100% (hypothetical value), relative or compared to vehicle, means that the rat does not eat.

In one embodiment the GLP-1-/GIP-/amylin-receptor tri-agonist disclosed herein, after a single subcutaneous administration of 10 nmol/kg, may reduce food intake at day 1 (0-24 hours) by at least 10% compared to vehicle, preferably by at least 50% compared to vehicle, such as at least 70% compared to vehicle. The GLP-1-/GIP-/amylin-receptor tri-agonist disclosed herein, after a single subcutaneous administration of 10 nmol/kg, may reduce food intake at day 1 (0-24 hours) by 1% to 100%, when compared to vehicle, such as 15% to 95%, preferably by 40% to 85% when compared to vehicle, even more preferably by 50% to 80%, when compared to vehicle.

In one embodiment the GLP-1-/GIP-/amylin-receptor tri-agonist disclosed herein, after a single subcutaneous administration of 10 nmol/kg, may reduce food intake at day 2 (24-48 hours) by at least 15% compared to vehicle, preferably by at least 50% compared to vehicle, such as at least 70% compared to vehicle. The GLP-1-/GIP-/amylin-receptor tri-agonist disclosed herein, after a single subcutaneous administration of 10 nmol/kg, may reduce food intake at day 2 (24-48 hours) by 1% to 100%, when compared to vehicle, such as 15% to 95%, preferably by 40% to 95% when compared to vehicle, even more preferably by 70% to 95%, when compared to vehicle.

In one embodiment the GLP-1-/GIP-/amylin-receptor tri-agonist disclosed herein, after a single subcutaneous administration of 30 nmol/kg, may reduce food intake at day 1 (0-24 hours) by at least 15% compared to vehicle, preferably by at least 35% compared to vehicle. The GLP-1-/GIP-/amylin-receptor tri-agonist disclosed herein, after a single subcutaneous administration of 30 nmol/kg, may reduce food intake at day 2 (24-48 hours) by at least 15% compared to vehicle, preferably by at least 35% compared to vehicle.

Both the peptide backbone and the protraction moiety have been engineered and refined to achieve a peptide derivative having all of the above properties.

Preferred GLP-1-/GIP-/amylin-receptor tri-agonists of the present invention are compound 52, that is:

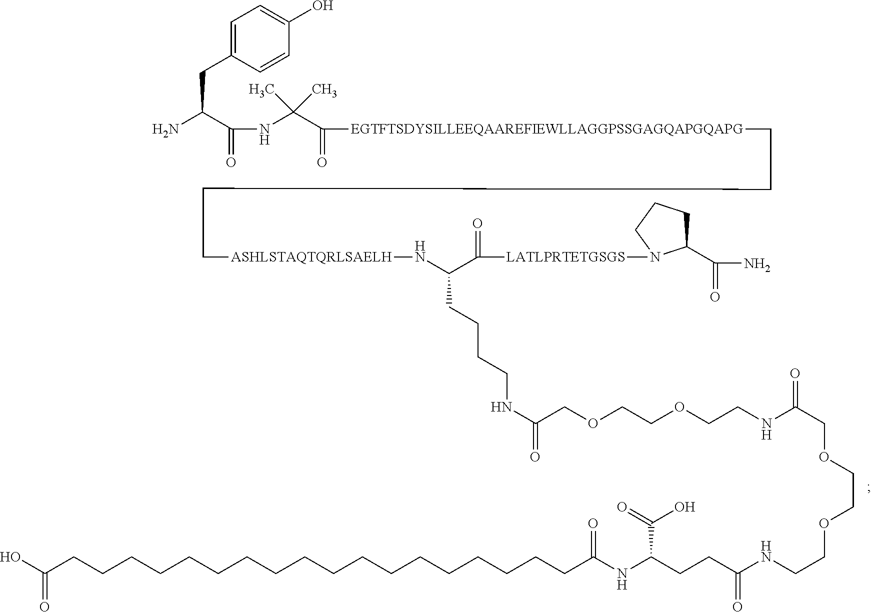

compound 55, that is:

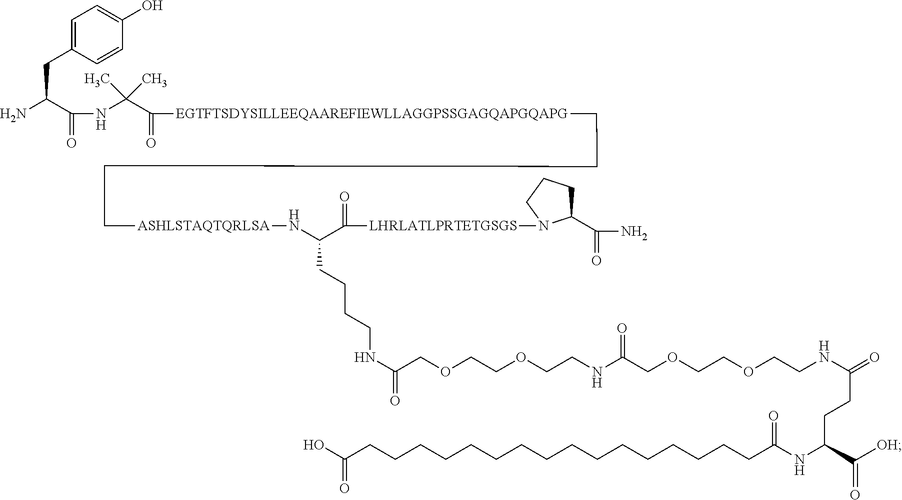

compound 58, that is:

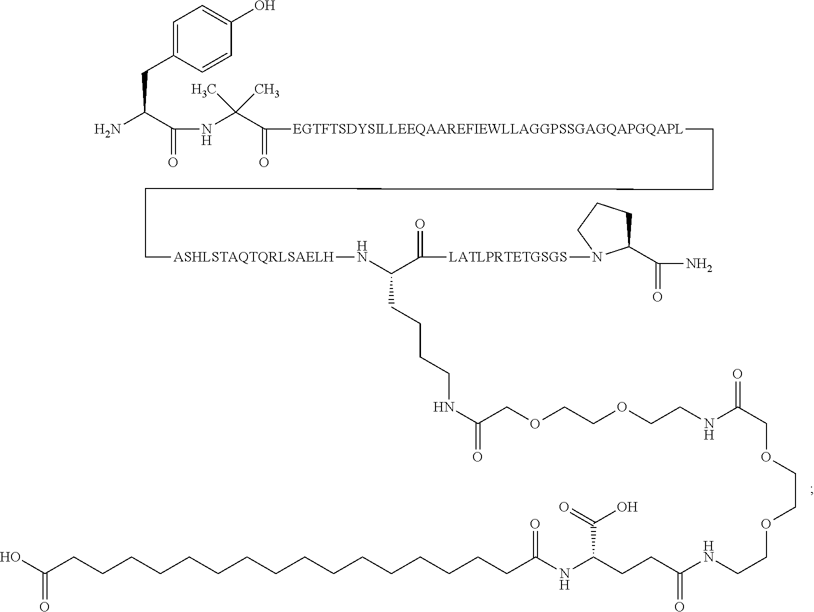

compound 68, that is:

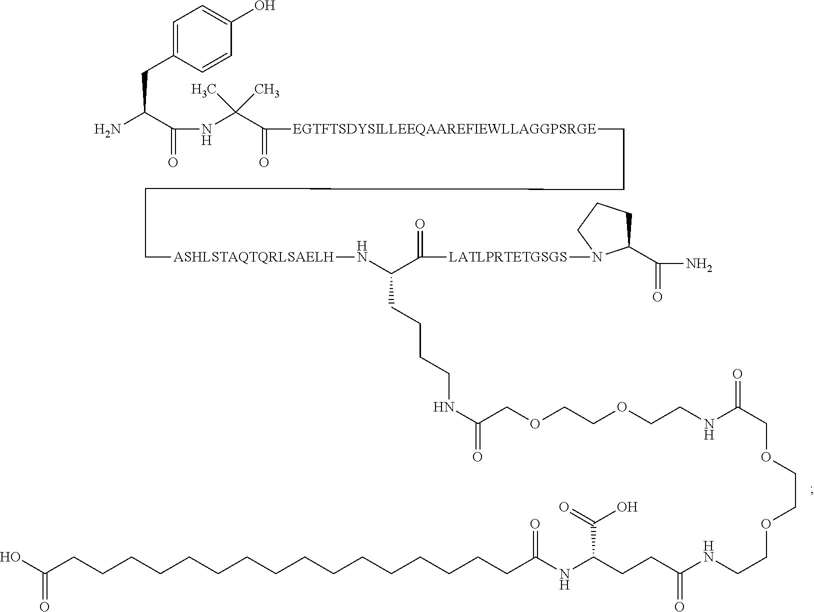

compound 77, that is:

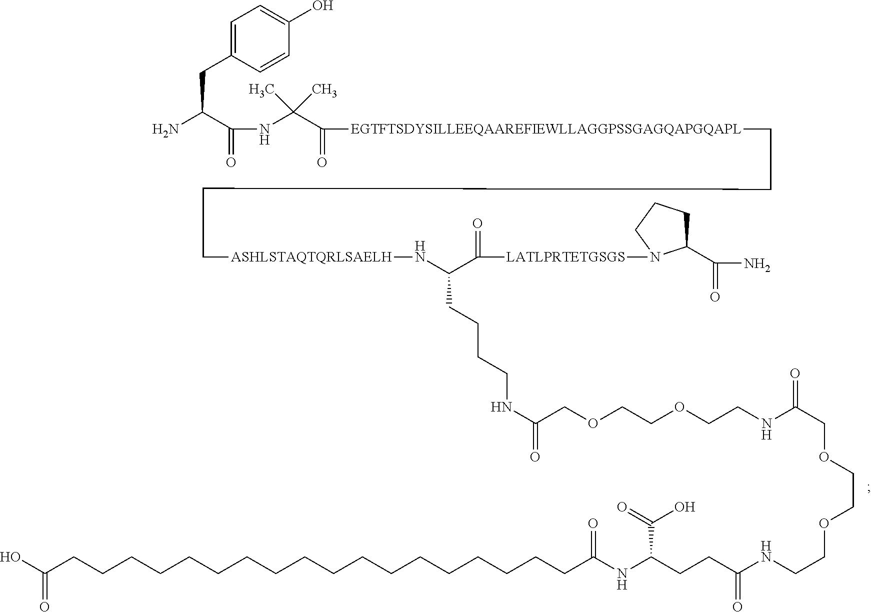

compound 101, that is:

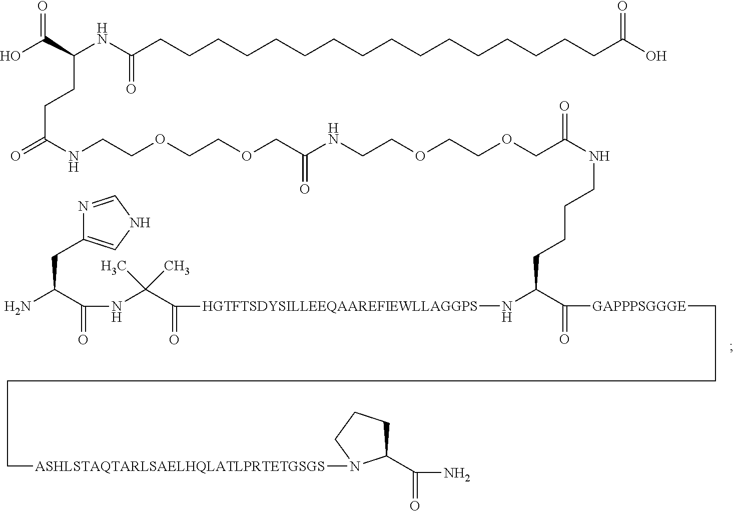

compound 210, that is:

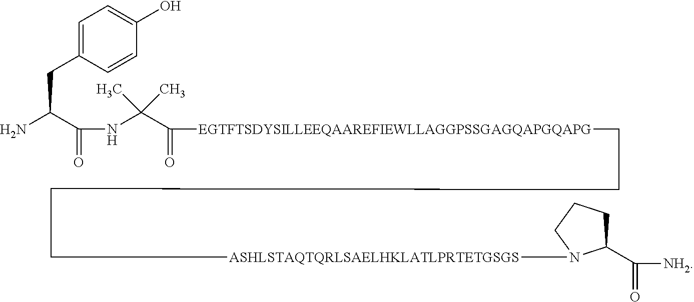

### Pharmaceutically Acceptable Salt

The compounds of the invention may be in the form of a pharmaceutically acceptable salt, or amide.

Salts are formed by a chemical reaction between a base and an acid, e.g.: 2NH₃+H₂SO₄→(NH₄)₂SO₄.

The salt may be a basic salt, an acid salt, or it may be neither nor (i.e., a neutral salt). Basic salts produce hydroxide ions and acid salts produce hydronium ions in water.

The salts of the compounds of the invention may be formed with added cations or anions between anionic or cationic groups, respectively. These groups may be situated in the peptide moiety, and/or in the protraction moiety of the compounds of the invention.

Non-limiting examples of anionic groups of the compounds of the invention include free carboxylic groups in the protraction moiety, if any, as well as in the peptide backbone.

The peptide backbone may include free carboxylic groups at internal amino acid residues such as Asp (D) and Glu (E).

Non-limiting examples of cationic groups in the peptide backbone include the free amino group at the N-terminus, if present, as well as any free amino group of internal basic amino acid residues such as His (H), Arg (R), and Lys (K).

The amide of the compound of the invention may, e.g., be formed during peptide synthesis (based on the used resin) or by the reaction of a free carboxylic acid group with an amine or a substituted amine, or by reaction of a free or substituted amino group with a carboxylic acid. The amide formation may be at any free carboxylic group in the protraction moiety, the free amino group at the N-terminus of the peptide, and/or any free or substituted amino group in the peptide backbone.

In one aspect, the derivative of the invention is in the form of a pharmaceutically acceptable salt, preferably in the form of a trifluoroacetate salt.

### Methods of Production

The tri-agonists disclosed herein may be produced by classical peptide synthesis, e.g. solid phase peptide synthesis using t-Boc or Fmoc chemistry, or other well established techniques, see e.g. Greene and Wuts, “Protective Groups in Organic Synthesis”, John Wiley & Sons, 1999; Florencio Zaragoza Dorwald, “Organic Synthesis on Solid Phase”, Wiley-VCH Verlag GmbH, 2000; and “Fmoc Solid Phase Peptide Synthesis”, Edited by W. C. Chan and P. O. White, Oxford University Press, 2000. In some embodiments, methods for preparing the triple agonists are described herein. In some embodiments, the methods for preparing the triple agonists as described herein comprises a step of solid phase peptide synthesis.

Also, or alternatively, the compounds, the peptide sequence or parts of the peptide sequences may be produced by recombinant methods, e.g., by culturing a host cell containing a DNA sequence encoding the tri-agonist peptide sequence and capable of expressing the peptide, in a suitable nutrient medium under conditions permitting the expression of the peptide. Non-limiting examples of host cells suitable for expression of these peptides are: Escherichia coli, Saccharomyces cerevisiae as well as mammalian BHK or CHO cell lines.

The tri-agonists that include non-natural amino acids and/or covalently attached substituents (protraction moieties) may be produced as described under ‘General method for peptide synthesis’ in the experimental part. Or see e.g., Hodgson et al: “The synthesis of peptides and proteins containing non-natural amino acids”, Chemical Society Reviews, vol. 33, no. 7 (2004), p. 422-430.

The tri-agonists as described herein which include a protraction moiety may, e.g., be produced as described under ‘General method for peptide synthesis’ in the experimental part. In some embodiments, the protraction moiety is built as part of the solid phase peptide synthesis or produced separately and attached via the one lysine residue after the solid phase peptide synthesis.

Specific examples of methods of preparing a number of the tri-agonists as described herein are provided below.

A further aspect of the invention relates to a method for preparing the receptor tri-agonists described herein.

In one embodiment, the method for preparing a compound as described herein comprises a step of solid phase peptide synthesis. The protraction moiety may be built sequentially as part of the solid phase peptide synthesis or produced separately and attached via the lysine residue after peptide synthesis.

### Pharmaceutical Compositions

In a further aspect the present invention relates to a pharmaceutical composition comprising said GLP-1-/GIP-/amylin-receptor triple agonist. Disclosed herein is a pharmaceutical composition comprising the GLP-1-/GIP-/amylin-receptor triple agonist as disclosed herein, and one or more pharmaceutically acceptable excipients. Pharmaceutical composition comprising the GLP-1-/GIP-/amylin-receptor triple agonist as disclosed herein, and one or more pharmaceutically acceptable excipients, may be prepared using methods known to the person skilled in the art.

The term “pharmaceutically acceptable excipient” refers to any ingredient in the pharmaceutical composition which is not the active pharmaceutical ingredient, or the GLP-1-/GIP-/amylin-receptor triple agonist as disclosed herein. The term “pharmaceutically acceptable excipient” means an excipient that is useful in preparing a pharmaceutical composition that is generally safe, non-toxic and includes excipients that are acceptable for human pharmaceutical use. Such excipients can for example be solid, liquid or semisolid.

The excipient may be functional or inert and may serve various purposes, e.g. as a buffer, an isotonicity agent, a carrier, a vehicle, a filler, a binder, a lubricant, a glidant, a disintegrant, a flow control agent, a crystallization inhibitor, a solubilizer, a stabilizer, a colouring agent, a flavouring agent, a surfactant, emulsifier, or combinations thereof and/or to improve administration, and/or absorption of the active pharmaceutical ingredient(s). The amount of each excipient used may vary within ranges conventional in the art.

Techniques and excipients which may be used are described in e.g., Handbook of Pharmaceutical Excipients (e.g., 8^{th} edition, Sheskey et al., Eds., American Pharmaceuticals Association and Pharmaceutical Press, publications department of the Royal Pharmaceutical Society of Great Britain (2017) and later editions) and Remington: The Science and Practice of Pharmacy (e.g., 23^{rd} edition, Remington and Allen, Eds., Pharmaceutical Press (2021) and later editions).

The pharmaceutical composition comprising the GLP-1-/GIP-/amylin-receptor triple agonist as disclosed herein may be for oral administration.

The pharmaceutical composition comprising the GLP-1-/GIP-/amylin-receptor triple agonist as disclosed herein may be a solid pharmaceutical composition (e.g., a tablet or capsule) containing the active pharmaceutical ingredient, for example as a freeze-dried or spray-dried composition, and may be used as is, dissolved prior to use, or combined with excipients in the formulation.

The pharmaceutical composition may be a solid pharmaceutical composition comprising the compound disclosed herein, a salt of N-[8-(2-hydroxybenzoyl)amino]caprylate, preferably sodium N-(8-(2-hydroxybenzoyl)amino)caprylate, and one or more further excipients, as is described in the art. For example, the solid pharmaceutical composition may be as described in WO 2012/080471, WO 2013/139694, WO 2013/189988, WO 2019/149880, WO 2019/215063, WO 2021/219710 or WO 2023/012263 A1.

Alternatively, the pharmaceutical composition comprising the GLP-1-/GIP-/amylin-receptor triple agonist as disclosed herein may be a liquid composition, such as an aqueous composition. Such liquid compositions may be suitable for oral administration or for parenteral administration, for example intravenous, intramuscular, or subcutaneous administration.

Liquid compositions that are suitable for injection can be prepared using conventional techniques of the pharmaceutical industry which involve dissolving and mixing the ingredients as appropriate to give the desired end product. Thus, according to one procedure, the compound described herein is dissolved in a suitable buffer at a suitable pH. The composition may be sterilized, for example, by sterile filtration. Techniques and excipients which may be used to prepare liquid formulations are described in in e.g., Handbook of Pharmaceutical Excipients (e.g., 8^{th} edition, Sheskey et al., Eds., American Pharmaceuticals Association and Pharmaceutical Press, publications department of the Royal Pharmaceutical Society of Great Britain (2017) and later editions) and Remington: The Science and Practice of Pharmacy (e.g., 23^{rd} edition, Remington and Allen, Eds., Pharmaceutical Press (2021) and later editions). Preferably, in an embodiment wherein pharmaceutical composition is in a liquid formulation, the liquid formulation provides an improved stability.

The pharmaceutical compositions are typically administered to a subject already suffering from a disease, such as the indications described below, in an amount sufficient to cure, alleviate or partially arrest the disease and its complications. An amount adequate to accomplish this is defined as “therapeutically effective amount”. As will be understood by the person skilled in the art amounts effective for this purpose will depend on the severity of the disease as well as the weight and general state of the subject.

In some embodiments the dose of the compounds to be delivered by subcutaneous administration may be from about 0.1 mg to 500 mg of the compound per day, preferably from about 0.5 mg to 150 mg per day, per every second day, per every third day, per every fourth day, per every fifth day, per every sixth day or once weekly depending on the severity of the condition.

A suitable dose may also be adjusted for a particular compound based on the properties of that compound, including its in vivo half-life or mean residence time and its biological activity. For example, compounds to be delivered could in one embodiment be administered once daily, or in another embodiment once weekly. Accordingly, the pharmaceutical compositions may be used for dosing approximately once daily, such as once every 12-36 hours, such as once every 18-30 hours, such as approximately once every 24 hours, or may be used for dosing approximately once weekly, such as once every 6-8 days.

In one embodiment the present invention relates to an injection device comprising said pharmaceutical composition.

### Indications

In a further aspect the present invention relates to the GLP-1-/GIP-/amylin-receptor triple agonist as disclosed herein for use as a medicament.

The GLP-1-/GIP-/amylin-receptor triple agonist as disclosed herein may be used for the following medical treatments or indications:
(i) prevention and/or treatment of all forms of diabetes, such as hyperglycaemia, type 2 diabetes, impaired glucose tolerance, type 1 diabetes, non-insulin dependent diabetes, MODY (maturity onset diabetes of the young), gestational diabetes, and/or for reduction of HbA1c;
(ii) delaying or preventing diabetic disease progression, such as progression in type 2 diabetes, delaying the progression of impaired glucose tolerance (IGT) to insulin requiring type 2 diabetes, and/or delaying the progression of non-insulin requiring type 2 diabetes to insulin requiring type 2 diabetes;
(iii) prevention and/or treatment of eating disorders, such as obesity, e.g., by decreasing food intake, reducing body weight, suppressing appetite, inducing satiety; treating or preventing binge eating disorder, food cravings, bulimia nervosa and/or obesity induced by administration of an antipsychotic or a steroid; reduction of gastric motility; and/or delaying gastric emptying;
(iv) weight maintenance after successful weight loss (either drug induced or by diet and exercise)—i.e., prevention of weight gain after successful weight loss;
(v) prevention and/or treatment of cardiovascular diseases, such as delaying or reducing development of a major adverse cardiovascular event (MACE) selected from the group consisting of cardiovascular death, non-fatal myocardial infarction, non-fatal stroke, revascularisation, hospitalisation for unstable angina pectoris, and hospitalisation for heart failure.
(vi) prevention and/or treatment of non-alcoholic fatty liver disease (NAFLD, otherwise known as metabolic-dysfunction associated fatty liver disease, MAFLD) and/or non-alcoholic steatohepatitis (NASH, otherwise known as metabolic dysfunction-associated steatohepatitis, MASH);
(vii) prevention and/or treatment of cognitive impairment, such as that caused by Alzheimer's disease;
(viii) the prevention and/or treatment of chronic kidney disease;
(ix) the prevention and/or treatment of obstructive sleep apnoea.

In some embodiments the indication is (i). In some embodiments the indication is (ii). In a still further particular aspect the indication is (iii). In some embodiments the indication is (iv). In some embodiments the indication is (v) In some embodiments the indication is (vi). In some embodiments the indication is (vii). In some embodiments the indication is (viii). In some embodiments the indication is (ix). In some embodiments the indication is type 2 diabetes. In some embodiments the indication is overweight or obesity.

The term “treatment”, as used herein, refers to the medical therapy of any human or other vertebrate subject in need thereof. Said subject is expected to have undergone physical examination by a medical practitioner, or a veterinary medical practitioner, who has given a tentative or definitive diagnosis which would indicate that the use of said specific treatment is beneficial to the health of said human or other vertebrate. The timing and purpose of said treatment may vary from one individual to another, according to the status quo of the subject's health. Thus, said treatment may be prophylactic (preventive), palliative, symptomatic and/or curative.

In some embodiments the indication is (i) and (iii). In some embodiments the indication is (ii) and (iii).

The World Health Organisation (WHO) defines overweight and obesity as being the abnormal or excessive accumulation of body fat that present a risk to an individual's overall health. Generally, all subjects suffering from obesity are also considered to be suffering from overweight. The subject suffering from obesity may be a human being, such as an adult human or a paediatric human, wherein “paediatric human” includes the infant, the child and the adolescents. Alternatively to overweight, also the term pre-obesity is used in this field.

The WHO considers body mass index (BMI) to be the most convenient population-level measure of overweight and obesity. Body mass index (BMI) is a measure of body fat based on height and weight. The formula for calculation is BMI=weight in kilograms (kg)/height in meters squared (m²).

For adults, the WHO defines overweight, and obesity as follows: overweight means having a BMI greater than or equal to 25; obesity means having a BMI greater than or equal to 30.

For children, the WHO considers age when defining overweight and obesity. For children under the age of five, overweight means having a weight-for-height greater than two standard deviations above the WHO Child Growth Standards median; and obesity means having a weight-for-height greater than three standard deviations above the WHO Child Growth Standards median. Overweight and obesity are defined as follows for children aged five to nineteen: overweight means having a BMI-for-age that is greater than one standard deviation above the WHO Growth Reference median; and obesity means having a BMI-for-age that is greater than two standard deviations above the WHO Growth Reference median.

Nonetheless, the diagnostic criteria for underweight, the normal range, pre-obesity/overweight and obesity can differ between countries/populations, as illustrated in Table 16 below for adults.

TABLE 16
Definitions of underweight, the normal range,
pre-obesity/overweight and obesity in adults

| | BMI (kg/m²) | | | | |
|---|---|---|---|---|---|
| | International/ | | | | |
| Nutritional | European/US | Chinese | Japanese | Taiwanese | Korean |
| status | classification | population | classification | classification | classification |
| Underweight | <18.5 | <18.5 | <18.5 | <18.5 | |
| Normal weight | >/=18.5 | >/=18.5 | >/=18.5 | >/=18.5 | |
| | and <25 | and <23 | and <25 | and <24 | |
| Overweight | >/=25 | >/=23 | | >/=24 | >/=23 |
| (pre-obesity) | and <30 | and <28 | | and <27 | and <25 |
| Obesity | >/=30 | >/=28 | >/=25 | >/=27 | >/=25 |
| Obesity class I | >/=30 | | >/=25 | >/=27 | >/=25 |
| | and <35 | | and <30 | and <30 | and <30 |
| Obesity class II | >/=35.0 | | >/=30 | >/=30 | >/=30 |
| | and <40 | | and <35 | and <35 | and <35 |
| Obesity class III | >/=40 | | >/=35 | >/=35 | >/=35 |
| | | | and <40 | | |
| Obesity class IV | | | >/=40 | | |

Guidelines for the Asian population were published by Misra A et al. J Assoc Physicians India. 2009; 57:163-70. Guidelines for the Chinese population were issued in the 2006 edition of the Guidelines for Prevention and Control of Overweight and Obesity in Chinese Adults, compiled by the Chinese Working Group on Obesity. Guidelines for the Japanese population were issued, in 2016, by the Japanese Society for the Study of Obesity (JASSO) in Guidelines for the management of obesity disease. Guidelines for the Taiwanese population were issued by the Taiwanese government's Health Promotion Administration (HPA), Ministry of Health and Welfare in 2023, in the 2^{nd} edition of its “Evidence-Based Guideline on Adult Obesity Prevention and Management”.

In some embodiments the subject suffering from obesity is human, such as an adult human or a paediatric human (including infants, children, and adolescents). A human subject suffering from obesity thus may have a BMI of 25 or more, or 27 or more, or 28 or more, or 30 or more; this subject may also be referred to as being obese. The obesity may be class I, class II, class III or class IV obesity (as defined in Table 16). In some embodiments the human subject suffering from obesity may have a BMI of ≥35 or a BMI in the range of ≥30 to <40. In some embodiments the obesity is severe obesity or morbid obesity, wherein the human subject may have a BMI of ≥40.

In some embodiments the invention relates to a method for treatment or prevention of overweight, optionally in the presence of at least one weight-related co-morbidity. In one embodiment the GLP-1-/GIP-/amylin-receptor triple agonist as disclosed herein is for use in the treatment of a subject with an initial body mass index (BMI) of 25 or more, 27 or more, or 28 or more, or 30 or more; optionally in the presence of at least one weight-related co-morbidity.

In some embodiments the invention relates to use of the formulation for treatment or prevention of overweight, optionally in the presence of at least one weight-related co-morbidity. In some embodiments the subject suffering from overweight is human, such as an adult human or a paediatric human (including infants, children, and adolescents). In some embodiments an adult human subject suffering from overweight may have a BMI of 23 or more, or 24 or more, or 25 or more, or 27 or more. In some embodiments a human subject suffering from overweight has a BMI in the range of 24 to <27, in the range of 24 to <28, in the range of 25 to <30 or in the range of 27 to <30. In some embodiments the weight-related co-morbidity is selected from the group consisting of hypertension, dysglycaemia (prediabetes or type 2 diabetes), dyslipidaemia, high cholesterol, cardiovascular disease and obstructive sleep apnoea.

In some embodiments, the tri-agonist as disclosed herein relates to a method for weight management. In some embodiments, the tri-agonist as disclosed herein relates to a method for reduction of appetite. In some embodiments, the tri-agonist as disclosed herein relates to a method for reduction of food intake. In some embodiments, the tri-agonist as disclosed herein relates to a method of preventing or treating overweight in a subject.

The term “reduction of body weight” may include treatment or prevention of obesity and/or overweight.

Administration of the compound disclosed herein may be as an adjunct to a reduced-calorie diet and increased physical activity for chronic weight management in an adult subject suffering from obesity, i.e. with an initial body mass index (BMI) of 25 or more, or 27 or more, or 28 or more, or 30 or more; or in an adult subject suffering from overweight, i.e. with an initial body mass index (BMI) of 23 or more, or 24 or more, or 25 or more, or 27 or more; optionally in the presence of at least one weight-related co-morbidity (e.g., hypertension, dysglycaemia (prediabetes or type 2 diabetes), dyslipidaemia, high cholesterol, cardiovascular disease or obstructive sleep apnoea).

### Methods of Production

The compounds disclosed herein may, for instance, be produced by classical peptide synthesis, e.g. solid phase peptide synthesis using t-Boc or Fmoc chemistry, or other well established techniques, see e.g. Greene and Wuts, “Protective Groups in Organic Synthesis”, John Wiley & Sons, 1999; Florencio Zaragoza Dörwald, “Organic Synthesis on Solid Phase”, Wiley-VCH Verlag GmbH, 2000; and “Fmoc Solid Phase Peptide Synthesis”, Edited by W. C. Chan and P. D. White, Oxford University Press, 2000.

Alternatively, the compounds may be produced by recombinant methods, e.g., by culturing a host cell containing a DNA sequence encoding the peptide sequence and capable of expressing the peptide, in a suitable nutrient medium under conditions permitting the expression of the peptide. Non-limiting examples of host cells suitable for expression of these peptides are Escherichia coli, Saccharomyces cerevisiae and mammalian BHK or CHO. Specific examples of methods of preparing the disclosed compounds are included in the examples.

A further aspect of the invention relates to a method for preparing the peptides described herein. In an embodiment, the method for preparing a compound as described herein comprises a step of solid phase peptide synthesis. The protraction moiety may be introduced sequentially as part of the solid phase peptide synthesis or produced separately and attached via the alanine or lysine residue after peptide synthesis.

### Particular Embodiments

1. A GLP-1-/GIP-/amylin-receptor tri-agonist comprising a peptide according to Formula I:

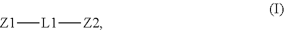

comprising one lysine (Lys, K) residue; wherein:
   Z1 is a peptide comprising a maximum of 4 amino acid substitutions relative to Formula II (SEQ ID NO: 1):

(II)
YX₂EGTFTSDYSILLEEQAAREFIEWLLAGGPSKG,

wherein the amino acid at position X₂ represents Aib;
   L1 is a peptide linker; and
   Z2 is a peptide comprising a C-terminal amide and a maximum of 10 amino acid substitutions relative to Formula III (SEQ ID NO: 2):

(III)
ASELSTAALGRLSAELHELATLPRTETGSGSP.

2. The GLP-1-/GIP-/amylin-receptor tri-agonist according to embodiment 11, wherein peptide Z1 comprises an amino acid sequence which has at least 87, 88, 89, 90, 91, 92, 93, 94, 95, 96, 97, 98 or 99% identity to Formula II (SEQ ID NO: 1), and peptide Z2 comprises an amino acid sequence which has at least 70, 71, 72, 73, 74, 75, 76, 77, 78, 79, 80, 81, 82, 83, 84, 85, 86, 87, 88, 89 90, 91, 92, 93, 94, 95, 96, 97, 98 or 99% identity relative to Formula III (SEQ ID NO: 2).
3. A GLP-1-/GIP-/amylin-receptor tri-agonist comprising a peptide according to Formula I:

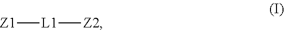

comprising one lysine (Lys, K) residue; wherein:
   Z1 is a peptide having a maximum of 4 amino acid substitutions relative to Formula II (SEQ ID NO: 1):

(II)
YX₂EGTFTSDYSILLEEQAAREFIEWLLAGGPSKG,

wherein the amino acid at position X₂ represents Aib,
and
Z1 comprises or consists of an amino acid sequence according to Formula X (SEQ ID NO: 161):

(X)
X₂₁X₂₂X₂₃GTFTSDYSX₂₄LLEEX₂₅AAX₂₆EFIX₂₇WLX₂₈X₂₉GGPSX₃₀X₃₁,

wherein
X₂₁ represents His (H) or Tyr (Y),
X₂₂ represents Aib,
X₂₃ represents Glu (E) or His (H),
X₂₄ represents Ile (I) or Lys (K),
X₂₅ represents Gln (Q) or Ile (I),
X₂₆ represents Arg (R) or Gln (Q),
X₂₇ represents Ala (A), Glu (E) or Gln (Q),
X₂₈ represents Leu (L) or I (Ile),
X₂₉ represents Ala (A) or Gln (Q),
X₃₀ represents Arg (R), Gly (G), Lys (K) or Ser (S),
X₃₁ represents Gly (G), Glu (E) or Lys (K);
L1 is a peptide linker; and
Z2 is a peptide comprising a C-terminal amide, and having a maximum of 10 amino acid substitutions relative to Formula III (SEQ ID NO: 2):

(III)
ASELSTAALGRLSAELHELATLPRTETGSGSP,

and
Z2 comprises or consists of an amino acid sequence according to Formula XII (SEQ ID NO:164):

(XII)
AX₃₂X₃₃LSTAX₃₄X₃₅X₃₆RLSAX₃₇LHX₃₈LX₃₉X₄₀X₄₁PX₄₂TETGSGX₄₃P,

wherein
X₃₂ represents Gly (G) or Ser (S),
X₃₃ represents Gln (Q), Glu (E), His (H) or Lys (K),
X₃₄ represents Ala (A) or Gln (Q),
X₃₅ represents Gln (Q), Leu (L) or Thr (T),
X₃₆ represents Ala (A), Gly (G) or Gln (Q),
X₃₇ represents Glu (E) or Lys (K),
X₃₈ represents Arg (R), Gln (Q), Glu (E), Gly (G), His (H), Lys (K), Thr (T) or Tyr (Y),
X₃₉ represents Ala (A) or Lys (K),
X₄₀ represents Asp (D) or Thr (T),
X₄₁ represents Leu (L) or Glu (E),
X₄₂ represents Arg (R) or Lys (K),
X₄₃ represents Ala (A) or Ser (S).
4. The GLP-1-/GIP-/amylin-receptor tri-agonist according to embodiment 1 to embodiment 3, wherein the one lysine (Lys, K) residue is present in the peptide Z1 or in the peptide Z2.
5. The GLP-1-/GIP-/amylin-receptor tri-agonist according to embodiment 1 to 4, wherein the GLP-1-/GIP-/amylin-receptor triple agonist does not comprise a cysteine (Cys, C) residue.
6. The GLP-1-/GIP-/amylin-receptor tri-agonist according to embodiments 1 to 5, wherein the GLP-1-/GIP-/amylin-receptor triple agonist does not comprise a disulfide bridge.
7. The GLP-1-/GIP-/amylin-receptor tri-agonist according to embodiments 1 to 6, wherein:
   Z1 is a peptide having a maximum of 4 amino acid substitutions relative to Formula II (SEQ ID NO: 1):

(II)
YX₂EGTFTSDYSILLEEQAAREFIEWLLAGGPSKG,

wherein the amino acid at position X₂ represents Aib,
and
Z1 comprises or consists of an amino acid sequence according to Formula lIa (SEQ ID NO: 3):

(IIa)
X₁X₂X₃GTFTSDYSILLEEQAAREFIEWLLAGGPSX₄X₅,

wherein
X₁ represents His (H) or Tyr (Y),
X₂ represents Aib,
X₃ represents Glu (E) or His (H),
X₄ represents Arg (R), Gly (G), Lys (K) or Ser (S),
X₅ represents Gly (G) or Lys (K);
L1 is a peptide linker; and
Z2 is a peptide comprising a C-terminal amide, and having a maximum of 10 amino acid substitutions relative to Formula III (SEQ ID NO: 2):

(III)
ASELSTAALGRLSAELHELATLPRTETGSGSP,

and
Z2 comprises or consists of an amino acid sequence according to Formula IIIa (SEQ ID NO: 4):

(IIIa)
ASX₆LSTAX₇X₈X₉RLSAX₁₀LHX₁₁LX₁₂X₁₃LPX₁₄TETGSGX₁₅P,

wherein
X₆ represents Gln (Q), Glu (E), His (H) or Lys (K),
X₇ represents Ala (A) or Gln (Q),
X₈ represents Gln (Q), Leu (L) or Thr (T),
X₉ represents Ala (A), Gly (G) or Gln (Q),
X₁₀ represents Glu (E) or Lys (K),
X₁₁ represents Arg (R), Gln (Q), Glu (E), Gly (G), His (H), Lys (K), Thr (T) or Tyr (Y),
X₁₂ represents Ala (A) or Lys (K),
X₁₃ represents Asp (D) or Thr (T),
X₁₄ represents Arg (R) or Lys (K),
X₁₅ represents Ala (A) or Ser (S).
8. The GLP-1-/GIP-/amylin-receptor tri-agonist according to embodiments 1 to 6, wherein:
   Z1 is a peptide having a maximum of 4 amino acid substitutions relative to Formula II (SEQ ID NO: 1):

(II)
YX₂EGTFTSDYSILLEEQAAREFIEWLLAGGPSKG,

wherein the amino acid at position X₂ represents Aib,
and
Z1 comprises or consists of an amino acid sequence according to Formula Xa SEQ ID NO: 162):

(Xa)
YX₅₁EGTFTSDYSX₅₂LLEEIAAX₅₃EFIX₅₄WLX₅₅X₅₆GGPSSX₅₇,

wherein
X₅₁ represents Aib,
X₅₂ represents Ile (i) or Lys (K),
X₅₃ represents Arg (R) or Gln (Q),
X₅₄ represents Ala (A), Glu (E) or Gln (Q),
X₅₅ represents Leu (L) or I (Ile),
X₅₆ represents Ala (A) or Gln (Q),
X₅₇ represents Gly (G) or Glu (E);
L1 is a peptide linker; and
Z2 is a peptide comprising a C-terminal amide, and having a maximum of 10 amino acid substitutions relative to Formula III (SEQ ID NO: 2):

(III)
ASELSTAALGRLSAELHELATLPRTETGSGSP,

and
Z2 comprises or consists of an amino acid sequence according to Formula XIIa (SEQ ID NO: 165):

(XIIa)
AX₅₈X₅₉LSTAX₆₀X₆₁X₆₂RLSAELHX₆₃LATX₆₄PRTETGSGSP.

wherein
X₅₈ represents Gly (G) or Ser (S),
X₅₉ represents Gln (Q), Glu (E), or His (H),
X₆₀ represents Ala (A) or Gln (Q),
X₆₁ represents Leu (L) or Thr (T),
X₆₂ represents Ala (A), Gly (G) or Gln (Q),
X₆₃ represents Gln (Q), Glu (E), or Lys (K),
X₆₄ represents Leu (L) or Glu (E).
9. The GLP-1-/GIP-/amylin-receptor tri-agonist according to any one of embodiments 1 to 8, wherein the peptide linker L1 comprises 1 to 14, 1 to 10, 4 to 10 or 9 to 10 amino acid residues.
10. The GLP-1-/GIP-/amylin-receptor tri-agonist according to any one of embodiments 1 to 9, wherein the peptide linker L1 comprises 1 to 14 amino acid residues selected from the group consisting of Ala (A), Glu (E), Gln (Q), Gly (G), Leu (L), Phe (F), Pro (P), Ser (S), Thr (T), Val (V), Asn (N).
11. The GLP-1-/GIP-/amylin-receptor tri-agonist according to any one of embodiments 1 to 10, wherein the peptide linker L1 comprises or consists of the amino acid sequence according to Formula IV:

(IV)
X₁X₂X₃X₄X₅X₆X₇X₈X₉X₁₀X₁₁X₁₂X₁₃X₁₄,

wherein
   X₁ represents Ala (A), Glu (E), Gly (G),
   X₂ represents Gln (Q), Glu (E), Gly (G), Leu (L), Pro (P) or is absent,
   X₃ represents Ala (A), Gln (Q), Glu (E), Gly (G), Pro (P) or absent,
   X₄ represents Ala (A), Gln (Q), Glu (E), Gly (G), Pro (P) or absent,
   X₅ represents Glu (E), Gly (G), Pro (P), Ser (S), Thr (T) or is absent,
   X₆ represents Glu (E), Gly (G), Leu (L), Gln (Q) or is absent,
   X₇ represents Ala (A), Gln (Q), Glu (E), Gly (G), Phe (F) or is absent,
   X₈ represents Ala (A), Gln (Q), Glu (E), Gly (G), Thr (T), Pro (P), Val (V) or is absent,
   X₉ represents Glu (E), Asn (N), Pro (P), Thr (T) or is absent,
   X₁₀ represents Ala (A), Gln (Q), Glu (E), Gly (G), Leu (L), Pro (P), Ser (S), Val (V) or is absent,
   X₁₁ represents Ala (A) or is absent,
   X₁₂ represents Gln (Q) or is absent,
   X₁₃ represents Thr (T) or is absent,
   X₁₄ represents Leu (L) or is absent.
12. The GLP-1-/GIP-/amylin-receptor tri-agonist according to any one of embodiments 1 to 11,
   wherein the peptide linker L1 comprises or consists of the amino acid sequence according to Formula IV:

(IV)
X₁X₂X₃X₄X₅X₆X₇X₈X₉X₁₀X₁₁X₁₂X₁₃X₁₄,
(SEQ ID NO: 125)
which is selected from the group consisting of
A, E, G, AE, AG, GE, APPE
(SEQ ID NO: 126)
GGGE,
(SEQ ID NO: 127)
AGQAPG,
(SEQ ID NO: 128)
APPPSGGG,
(SEQ ID NO: 129)
APPPSGGGE,
(SEQ ID NO: 130)
APPPSGGGG,
(SEQ ID NO: 131)
ALAQTLAQTL,
(SEQ ID NO: 132)
ALAQTLFVNQ,
(SEQ ID NO: 133)
ALAQTLGTNE,
(SEQ ID NO: 134)
ALQAPGQAPG,
(SEQ ID NO: 135)
ALQAPGQAPL,
(SEQ ID NO: 136)
AGQAPGQAPG,
(SEQ ID NO: 137)
AGQAPGQAPL,
(SEQ ID NO: 138)
GGGEGGGEGE,
(SEQ ID NO: 139)
GQAPGQAPGE,
(SEQ ID NO: 140)
GQEPGQEPGE,
(SEQ ID NO: 141)
APPPSLAQTLAQTL,
(SEQ ID NO: 142)
AGGGG,
(SEQ ID NO: 143)
AGEAPGQAPG,
(SEQ ID NO: 144)
AGEAPGEAPG,
(SEQ ID NO: 145)
AGQAPGQAPA,
(SEQ ID NO: 146)
AGQAPGQAPE,
(SEQ ID NO: 147)
AGQAPGQAPP,
(SEQ ID NO: 148)
AGQAPGQAPS,
(SEQ ID NO: 149)
AGQAPGQAPV,
(SEQ ID NO: 150)
EGQAPGQAPG,
(SEQ ID NO: 151)
AGQEPGQAPG,
(SEQ ID NO: 152)
AGQAEGQAPG,
(SEQ ID NO: 153)
AGQAPEQAPG,
(SEQ ID NO: 154)
AGQAPGEAPG,
(SEQ ID NO: 155)
AGQAPGQEPG,
(SEQ ID NO: 156)
AGQAPGQAEG,
(SEQ ID NO: 157)
AGQEPGQEPG,
(SEQ ID NO: 158)
AGQAPGQAP
and
(SEQ ID NO: 159)
AGQAPGEAPL.

13. The GLP-1-/GIP-/amylin-receptor tri-agonist according to embodiment 12, wherein the amino acid sequence according to Formula IV:

(IV)
X₁X₂X₃X₄X₅X₆X₇X₈X₉X₁₀X₁₁X₁₂X₁₃X₁₄

is selected from the group consisting of E, GE, APPPSGGGE (SEQ ID NO: 129), AGQAPGQAPG (SEQ ID NO: 136) AGQAPGQAPL (SEQ ID NO: 137), and AGQAPGEAPG (SEQ ID NO: 154).
14. The GLP-1-/GIP-/amylin-receptor tri-agonist according to any one of embodiments 1 to 7 and 9 to 13, wherein the peptide Z1 comprises or consists of the amino acid sequence according to Formula IIa SEQ ID NO: 3):

(IIa)
X₁X₂X₃GTFTSDYSILLEEQAAREFIEWLLAGGPSX₄X₅,

wherein
   X₁ represents His (H) or Tyr (Y),
   X₂ represents Aib,
   X₃ represents Glu (E) or His (H),
   X₄ represents Arg (R), Gly (G) or Ser (S),
   X₅ represents Gly (G); and
wherein the peptide Z2 comprises or consists of the amino acid sequence according to Formula IIIa (SEQ ID NO: 4):

(IIIa)
ASX₆LSTAX₇X₈X₉RLSAX₁₀LHX₁₁LX₁₂X₁₃LPX₁₄TETGSGX₁₅P,

wherein
   X₆ represents Gln (Q), Glu (E), His (H) or Lys (K),
   X₇ represents Ala (A) or Gln (Q),
   X₈ represents Gln (Q), Leu (L) or Thr (T),
   X₉ represents Ala (A), Gly (G) or Gln (Q),
   X₁₀ represents Glu (E) or Lys (K),
   X₁₁ represents Arg (R), Gln (Q), Glu (E), Gly (G), His (H), Lys (K), Thr (T) or Tyr (Y),
   X₁₂ represents Ala (A) or Lys (K),
   X₁₃ represents Asp (D) or Thr (T),
   X₁₄ represents Arg (R) or Lys (K),
   X₁₅ represents Ala (A) or Ser (S).
15. The GLP-1-/GIP-/amylin-receptor tri-agonist according to any one of embodiments 1 to 7 and 9 to 14,
   wherein the peptide Z2 comprises or consists of the amino acid sequence according to Formula IIIa (SEQ ID NO: 4):

(IIIa)
ASX₆LSTAX₇X₈X₉RLSAX₁₀LHX₁₁LX₁₂X₁₃LPX₁₄TETGSGX₁₅P,

wherein
   X₆ represents Lys (K),
   X₇ represents Ala (A) or Gln (Q),
   X₈ represents Gln (Q), Leu (L) or Thr (T),
   X₉ represents Ala (A), Gly (G) or Gln (Q),
   X₁₀ represents Glu (E),
   X₁₁ represents Arg (R), Gln (Q), Glu (E), Gly (G), His (H), Thr (T) or Tyr (Y),
   X₁₂ represents Ala (A),
   X₁₃ represents Asp (D) or Thr (T),
   X₁₄ represents Arg (R),
   X₁₅ represents Ala (A) or Ser (S);
or
   X₆ represents Gln (Q), Glu (E), or His (H),
   X₇ represents Ala (A) or Gln (Q),
   X₈ represents Gln (Q), Leu (L) or Thr (T),
   X₉ represents Ala (A), Gly (G) or Gln (Q),
   X₁₀ represents Lys (K),
   X₁₁ represents Arg (R), Gln (Q), Glu (E), Gly (G), His (H), Thr (T) or Tyr (Y),
   X₁₂ represents Ala (A),
   X₁₃ represents Asp (D) or Thr (T),
   X₁₄ represents Arg (R),
   X₁₅ represents Ala (A) or Ser (S);
or
   X₆ represents Gln (Q), Glu (E) or His (H),
   X₇ represents Ala (A) or Gln (Q),
   X₈ represents Gln (Q), Leu (L) or Thr (T),
   X₉ represents Ala (A), Gly (G) or Gln (Q),
   X₁₀ represents Glu (E),
   X₁₁ represents Lys (K),
   X₁₂ represents Ala (A),
   X₁₃ represents Asp (D) or Thr (T),
   X₁₄ represents Arg (R),
   X₁₅ represents Ala (A) or Ser (S);
or
   X₆ represents Gln (Q), Glu (E) or His (H),
   X₇ represents Ala (A) or Gln (Q),
   X₈ represents Gln (Q), Leu (L) or Thr (T),
   X₉ represents Ala (A), Gly (G) or Gln (Q),
   X₁₀ represents Glu (E),
   X₁₁ represents Arg (R), Gln (Q), Glu (E), Gly (G), His (H), Thr (T) or Tyr (Y),
   X₁₂ represents Lys (K),
   X₁₃ represents Asp (D) or Thr (T),
   X₁₄ represents Arg (R),
   X₁₅ represents Ala (A) or Ser (S);
or
   X₆ represents Gln (Q), Glu (E) or His (H),
   X₇ represents Ala (A) or Gln (Q),
   X₈ represents Gln (Q), Leu (L) or Thr (T),
   X₉ represents Ala (A), Gly (G) or Gln (Q),
   X₁₀ represents Glu (E),
   X₁₁ represents Arg (R), Gln (Q), Glu (E), Gly (G), His (H), Thr (T) or Tyr (Y),
   X₁₂ represents Ala (A),
   X₁₃ represents Asp (D) or Thr (T),
   X₁₄ represents Lys (K),
   X₁₅ represents Ala (A) or Ser (S).
16. The GLP-1-/GIP-/amylin-receptor tri-agonist according to any one of embodiments 1 to 7 and 9 to 15, wherein the peptide Z1 comprises or consists of the amino acid sequence according to Formula IIa (SEQ ID NO: 3):

(IIa)
X₁X₂X₃GTFTSDYSILLEEQAAREFIEWLLAGGPSX₄X₅,

wherein
   X₁X₂X₃ represents YAibE (Tyr-Aib-Glu) or HAibH (His-Aib-His)
   X₄ represents Arg (R), Gly (G) or Ser (S),
   X₅ represents Gly (G).
17. The GLP-1-/GIP-/amylin-receptor tri-agonist according to embodiment 16,
   wherein the peptide Z1 comprises or consists of the amino acid sequence according to Formula V SEQ ID NO: 6):

(V)
YX₂EGTFTSDYSILLEEQAAREFIEWLLAGGPSX₄G,

wherein
   X₂ represents Aib,
   X₄ is Arg (R) or Ser (S).
18. The GLP-1-/GIP-/amylin-receptor tri-agonist agonist according to any one of embodiments 1 to 7 and 9 to 15,
   wherein the peptide Z1 comprises or consists of the amino acid sequence according to Formula IIa SEQ ID NO: 3):

(IIa)
X₁X₂X₃GTFTSDYSILLEEQAAREFIEWLLAGGPSX₄X₅,

wherein
   X₁ represents His (H) or Tyr (Y),
   X₂ represents Aib,
   X₃ represents Glu (E) or His (H),
   X₄ represents Arg (R), Gly (G) or Ser (S),
   X₅ represents Gly (G); and
wherein the peptide Z2 comprises or consists of the amino acid sequence according to Formula IIIa (SEQ ID NO: 4):

(IIIa)
ASX₆LSTAX₇X₈X₉RLSAX₁₀LHX₁₁LX₁₂X₁₃LPX₁₄TETGSGX₁₅P,

wherein
   X₆ represents Glu (E) or His (H),
   X₇ represents Ala (A) or Gln (Q),
   X₈ represents Gln (Q), Leu (L) or Thr (T),
   X₉ represents Ala (A), Gly (G) or Gln (Q),
   X₁₀ represents Glu (E) or Lys (K),
   X₁₁ represents Arg (R), Gln (Q) or Lys (K),
   X₁₂ represents Ala (A) or Lys (K),
   X₁₃ represents Thr (T),
   X₁₄ represents Arg (R) or Lys (K),
   X₁₅ represents Ala (A) or Ser (S).
19. The GLP-1-/GIP-/amylin-receptor tri-agonist agonist according to any one of embodiments 1 to 7, 9 to 15 or embodiment 18,
   wherein the peptide Z1 comprises or consists of the amino acid sequence according to Formula IIa (SEQ ID NO: 3):

(IIa)
X₁X₂X₃GTFTSDYSILLEEQAAREFIEWLLAGGPSX₄X₅,

wherein
   X₁ represents His (H) or Tyr (Y),
   X₂ represents Aib,
   X₃ represents Glu (E) or His (H),
   X₄ represents Arg (R), Gly (G) or Ser (S),
   X₅ represents Gly (G); and
wherein the peptide Z2 comprises or consists of the amino acid sequence according to Formula IIIa (SEQ ID NO: 4):

(IIIa)
ASX₆LSTAX₇X₈X₉RLSAX₁₀LHX₁₁LX₁₂X₁₃LPX₁₄TETGSGX₁₅P,

wherein
   X₆ represents Glu (E) or His (H),
   X₇ represents Ala (A) or Gln (Q),
   X₈ represents Gln (Q), Leu (L) or Thr (T),
   X₉ represents Ala (A), Gly (G) or Gln (Q),
   X₁₀ represents Glu (E),
   X₁₁ represents Lys (K),
   X₁₂ represents Ala (A),
   X₁₃ represents Thr (T),
   X₁₄ represents Arg (R),
   X₁₅ represents Ala (A) or Ser (S).
20. The GLP-1-/GIP-/amylin-receptor tri-agonist agonist according to any one of embodiments 9 to 15, 18 and 19,
   wherein the peptide Z1 comprises or consists of the amino acid sequence according to Formula IIa (SEQ ID NO: 3):

(IIa)
X₁X₂X₃GTFTSDYSILLEEQAAREFIEWLLAGGPSX₄X₅,

wherein
   X₁ represents His (H) or Tyr (Y),
   X₂ represents Aib,
   X₃ represents Glu (E) or His (H),
   X₄ represents Arg (R), Gly (G) or Ser (S),
   X₅ represents Gly (G); and
wherein the peptide Z2 comprises or consists of the amino acid sequence according to Formula VIII (SEQ ID NO: 9):

(VIII)
ASHLSTAQTQRLSAELHKLATLPRTETGSGSP.

21. The GLP-1-/GIP-/amylin-receptor tri-agonist according to any one of embodiments 1 to 7 and 9 to 20, wherein the amino acid sequence of the peptide Z1-L1-Z2 comprises or consists of

(SEQ ID NO: 62)
YXEGTFTSDYSILLEEQAAREFIEWLLAGGPSSGAGQAPGQAPGASHLS
TAQTQRLSAELHKLATLPRTETGSGSP,
or
(SEQ ID NO: 68)
YXEGTFTSDYSILLEEQAAREFIEWLLAGGPSSGAGQAPGQAPLASHLS
TAQTQRLSAELHKLATLPRTETGSGSP,
or
(SEQ ID NO: 78)
YXEGTFTSDYSILLEEQAAREFIEWLLAGGPSRGEASHLSTAQTQRLSA
ELHKLATLPRTETGSGSP,
or
(SEQ ID NO: 87)
YXEGTFTSDYSILLEEQAAREFIEWLLAGGPSSGAGQAPGQAPLASHLS
TAQTQRLSAELHKLATLPRTETGSGSP;

wherein in each amino acid sequence X represents Aib.
22. The GLP-1-/GIP-/amylin-receptor tri-agonist according to embodiments 16 or 17, wherein the peptide Z2 comprises or consists of the amino acid sequence according to Formula VII SEQ ID NO: 8):

(VII)
ASHLSTAQTQRLSAKLHRLATLPRTETGSGSP.

23. The GLP-1-/GIP-/amylin-receptor tri-agonist according to embodiment 22, wherein the amino acid sequence of the peptide Z1-L1-Z2 comprises or consists of

(SEQ ID NO: 65)
YXEGTFTSDYSILLEEQAAREFIEWLLAGGPSSGAGQAPGQAPGASHL
STAQTQRLSAKLHRLATLPRTETGSGSP.

24. The GLP-1-/GIP-/amylin-receptor tri-agonist according to any one of embodiments 1 to 7, 9 to 15 and 18,
   wherein the peptide Z1 comprises or consists of the amino acid sequence according to Formula IIa (SEQ ID NO: 3):

(IIa)
X₁X₂X₃GTFTSDYSILLEEQAAREFIEWLLAGGPSX₄X₅,

wherein
   X₁ represents His (H) or Tyr (Y),
   X₂ represents Aib,
   X₃ represents Glu (E) or His (H),
   X₄ represents Arg (R), Gly (G) or Ser (S),
   X₅ represents Gly (G); and
wherein the peptide Z2 comprises or consists of the amino acid sequence according to Formula IIIa (SEQ ID NO: 4):

(IIIa)
ASX₆LSTAX₇X₈X₉RLSAX₁₀LHX₁₁LX₁₂X₁₃LPX₁₄TETGSGX₁₅P,

wherein
   X₆ represents His (H),
   X₇ represents Ala (A) or Gln (Q),
   X₈ represents Leu (L) or Thr (T),
   X₉ represents Ala (A), Gly (G) or Gln (Q),
   X₁₀ represents Glu (E),
   X₁₁ represents Arg (R) or Gln (Q),
   X₁₂ represents Lys (K),
   X₁₃ represents Thr (T),
   X₁₄ represents Arg (R),
   X₁₅ represents Ser (S).
25. The GLP-1-/GIP-/amylin-receptor tri-agonist according to any one of embodiments 1 to 7, 9 to 15 and 18, wherein the peptide Z1 comprises or consists of the amino acid sequence according to Formula IIa (SEQ ID NO: 3)

(IIa)
X₁X₂X₃GTFTSDYSILLEEQAAREFIEWLLAGGPSX₄X₅,

wherein
   X₁ represents His (H) or Tyr (Y),
   X₂ represents Aib,
   X₃ represents Glu (E) or His (H),
   X₄ represents Arg (R), Gly (G) or Ser (S),
   X₅ represents Gly (G); and
wherein the peptide Z2 comprises or consists of the amino acid sequence according to Formula IIIa (SEQ ID NO: 4):

(IIIa)
ASX₆LSTAX₇X₈X₉RLSAX₁₀LHX₁₁LX₁₂X₁₃LPX₁₄TETGSGX₁₅P,

wherein
   X₆ represents His (H),
   X₇ represents Ala (A) or Gln (Q),
   X₈ represents Leu (L) or Thr (T),
   X₉ represents Ala (A), Gly (G) or Gln (Q),
   X₁₀ represents Glu (E),
   X₁₁ represents Arg (R),
   X₁₂ represents Ala (A),
   X₁₃ represents Thr (T),
   X₁₄ represents Lys (K),
   X₁₅ represents Ser (S).
26. The GLP-1-/GIP-/amylin-receptor tri-agonist according to any one of embodiments 1 to 7, 9 to 15 and 18, wherein the peptide Z1 comprises or consists of the amino acid sequence according to Formula IIa (SEQ ID NO: 3):

(IIa)
X₁X₂X₃GTFTSDYSILLEEQAAREFIEWLLAGGPSX₄X₅,

wherein
   X₁ represents His (H) or Tyr (Y),
   X₂ represents Aib,
   X₃ represents Glu (E) or His (H),
   X₄ represents Arg (R), Gly (G) or Ser (S),
   X₅ represents Gly (G); and
wherein the peptide Z2 comprises or consists of the amino acid sequence according to Formula IIIa SEQ ID NO: 4):

(IIIa)
ASX₆LSTAX₇X₈X₉RLSAX₁₀LHX₁₁LX₁₂X₁₃LPX₁₄TETGSGX₁₅P,

wherein
   X₆ represents His (H),
   X₇ represents Gln (Q),
   X₈ represents Thr (T),
   X₉ represents Gln (Q),
   X₁₀ represents Glu (E),
   X₁₁ represents Arg (R) or Lys (K),
   X₁₂ represents Ala (A) or Lys (K),
   X₁₃ represents Thr (T),
   X₁₄ represents Arg (R) or Lys (K),
   X₁₅ represents Ser (S).
27. The GLP-1-/GIP-/amylin-receptor tri-agonist according to any one of embodiments 1 to 7 and 9 to 13,
   wherein the peptide Z1 comprises or consists of the amino acid sequence according to Formula IIa (SEQ ID NO: 3):

(IIa)
X₁X₂X₃GTFTSDYSILLEEQAAREFIEWLLAGGPSX₄X₅,

wherein
   X₁ represents His (H),
   X₂ represents Aib,
   X₃ represents His (H),
   X₄ represents Arg (R), Gly (G), Lys (K) or Ser (S),
   X₅ represents Gly (G); and
wherein the peptide Z2 comprises or consists of the amino acid sequence according to Formula IIIa SEQ ID NO: 4):

(IIIa)
ASX₆LSTAX₇X₈X₉RLSAX₁₀LHX₁₁LX₁₂X₁₃LPX₁₄TETGSGX₁₅P,

wherein
   X₆ represents Gln (Q), Glu (E), His (H) or Lys (K),
   X₇ represents Ala (A) or Gln (Q),
   X₈ represents Gln (Q), Leu (L) or Thr (T),
   X₉ represents Ala (A), Gly (G) or Gln (Q),
   X₁₀ represents Glu (E) or Lys (K),
   X₁₁ represents Arg (R), Gln (Q), Glu (E), Gly (G), His (H), Lys (K), Thr (T) or Tyr (Y),
   X₁₂ represents Ala (A) or Lys (K),
   X₁₃ represents Asp (D) or Thr (T),
   X₁₄ represents Arg (R) or Lys (K),
   X₁₅ represents Ala (A) or Ser (S).
28. The GLP-1-/GIP-/amylin-receptor tri-agonist according to embodiment 27, wherein the peptide Z1 comprises or consists of the amino acid sequence according to Formula IIa (SEQ ID NO: 3):

(IIa)
X₁X₂X₃GTFTSDYSILLEEQAAREFIEWLLAGGPSX₄X₅,

wherein
   X₁ represents His,
   X₂ represents Aib,
   X₃ represents His (H),
   X₄ represents Lys (K),
   X₅ represents Gly (G); and
wherein the peptide Z2 comprises or consists of the amino acid sequence according to Formula IIIa (SEQ ID NO: 4):

(IIIa)
ASX₆LSTAX₇X₈X₉RLSAX₁₀LHX₁₁LX₁₂X₁₃LPX₁₄TETGSGX₁₅P,

wherein
   X₆ represents Glu (E) or His (H),
   X₇ represents Ala (A) or Gln (Q),
   X₈ represents Leu (L) or Thr (T),
   X₉ represents Ala (A), Gly (G) or Gln (Q),
   X₁₀ represents Glu (E),
   X₁₁ represents Arg (R), Gln (Q) or Glu (E),
   X₁₂ represents Ala (A),
   X₁₃ represents Asp (D) or Thr (T),
   X₁₄ represents Arg (R),
   X₁₅ represents Ser (S).
29. The GLP-1-/GIP-/amylin-receptor tri-agonist according to embodiment 27 or embodiment 28,
   wherein in Formula IIIa (SEQ ID NO: 4)
      X₆ represents Glu (E) or His (H),
      X₇ represents Ala (A) or Gln (Q),
      X₈ represents Leu (L) or Thr (T),
      X₉ represents Ala (A) or Gly (G),
      X₁₀ represents Glu (E),
      X₁₁ represents Arg (R), Gln (Q) or Glu (E),
      X₁₂ represents Ala (A),
      X₁₃ represents Thr (T),
      X₁₄ represents Arg (R),
      X₁₅ represents Ser (S).
30. The GLP-1-/GIP-/amylin-receptor tri-agonist according to any one of embodiments 27 to 29,
   wherein the amino acid sequence of the peptide Z1-L1-Z2 comprises or consists of

(SEQ ID NO: 111)
HXHGTFTSDYSILLEEQAAREFIEWLLAGGPSKGAPPPSGGGEASHLST
AQTARLSAELHQLATLPRTETGSGSP, wherein X represents
Aib.

31. The GLP-1-/GIP-/amylin-receptor tri-agonist according to any one of embodiments 1 to 7 and 9 to 13,
   wherein the peptide Z1-L1-Z2 comprises or consists of an amino acid sequence selected from the group consisting of SEQ ID NOs 20 to 124, wherein X represents Aib.
32. The GLP-1-/GIP-/amylin-receptor tri-agonist according to any one of embodiments 7, 14, 15, 19, 20, 24, 25, 26, 27, or 28, wherein X₁ represents His (H).
33. The GLP-1-/GIP-/amylin-receptor tri-agonist according to any one of embodiments 7, 14, 18, 19, 20, 24, 25, or 26, wherein X₁ represents Tyr (Y).
34. The GLP-1-/GIP-/amylin-receptor tri-agonist according to any one of embodiments 7, 14, 19, 20, 24, 25, or 26, wherein X₃ represents Glu (E).
35. The GLP-1-/GIP-/amylin-receptor tri-agonist according to any one of embodiments 7, 14, 18, 19, 20, 24, 25, 26, 27, or 28, wherein X₃ represents His (H).
36. The GLP-1-/GIP-/amylin-receptor tri-agonist according to any one of embodiments 7, 14, 16, 17, 18, 19, 20, 24, 25, 26, or 27, wherein X₄ represents Arg (R).
37. The GLP-1-/GIP-/amylin-receptor tri-agonist according to any one of embodiments 7, 14, 16, 18, 19, 20, 24, 25, 26, or 27, wherein X₄ represents Gly (G).
38. The GLP-1-/GIP-/amylin-receptor tri-agonist according to any one of embodiments 7, 27, or 28, wherein X₄ represents Lys (K).
39. The GLP-1-/GIP-/amylin-receptor tri-agonist according to any one of embodiments 7, 14, 16, 17, 18, 20, 20, 24, 25, 26 or 27 wherein X₄ represents Ser (S).
40. The GLP-1-/GIP-/amylin-receptor tri-agonist according to any one of embodiments 7, 14, 16, 18, 19, 20, 24, 25, 26, 27 or 28, wherein X₅ represents Gly (G).
41. The GLP-1-/GIP-/amylin-receptor tri-agonist according to any one of embodiments 7, 14, 15, or 27, wherein X₅ represents Lys (K).
42. The GLP-1-/GIP-/amylin-receptor tri-agonist according to any one of embodiments 7, 14, 15, or 27, wherein X₆ represents Gln (Q).
43. The GLP-1-/GIP-/amylin-receptor tri-agonist according to any one of embodiments 7, 14, 15, 18, 19, 27, 28 or 29, wherein X₆ represents Glu (E).
44. The GLP-1-/GIP-/amylin-receptor tri-agonist according to any one of embodiments 7, 14, 15, 16, 19, 24, 25, 26, 27, 28 or 29, wherein X₆ represents His (H).
45. The GLP-1-/GIP-/amylin-receptor tri-agonist according to any one of embodiments 7, 14, 15, or 27, wherein X₆ represents Lys (K).
46. The GLP-1-/GIP-/amylin-receptor tri-agonist according to any one of embodiments 7, 14, 15, 18, 19, 24, 25, 26, 27, 28 or 29, wherein X₇ represents Ala (A).
47. The GLP-1-/GIP-/amylin-receptor tri-agonist according to any one of embodiments 7, 14, 15, 17, 19, 24, 25, 26, 27, 28 or 29, wherein X₇ represents Gln (Q).
48. The GLP-1-/GIP-/amylin-receptor tri-agonist according to any one of embodiments 7, 14, 15, 18, 19, or 27, wherein X₈ represents Gln (Q).
49. The GLP-1-/GIP-/amylin-receptor tri-agonist according to any one of embodiments 7, 14, 15, 18, 19, 24, 25, 27, 28 or 29, wherein X₈ represents Leu (L).
50. The GLP-1-/GIP-/amylin-receptor tri-agonist according to any one of embodiments 7, 14, 15, 18, 19, 24, 25, 26, 27, 28 or 29, wherein X₈ represents Thr (T).
51. The GLP-1-/GIP-/amylin-receptor tri-agonist according to any one of embodiments 7, 14, 15, 18, 19, 24, 25, 27, 28 or 29, wherein X₈ represents Ala (A).
52. The GLP-1-/GIP-/amylin-receptor tri-agonist according to any one of embodiments 7, 14, 15, 18, 19, 24, 25, 26, 27, 28 or 29, wherein X₈ represents Gly (G).
53. The GLP-1-/GIP-/amylin-receptor tri-agonist according to any one of embodiments 7, 14, 15, 18, 19, 24, 25, 26, 27 or 28, wherein X₈ represents Gln (Q).
54. The GLP-1-/GIP-/amylin-receptor tri-agonist according to any one of embodiments 7, 14, 15, 18, 19, 24, 25, 26, 27, 28 or 29, wherein X₁₀ represents Glu (E).
55. The GLP-1-/GIP-/amylin-receptor tri-agonist according to any one of embodiments 7, 14, 15, 18 or 27, wherein X₁₀ represents Lys (K).
56. The GLP-1-/GIP-/amylin-receptor tri-agonist according to any one of embodiments 7, 14, 15, 18, 24, 25, 26, 27, 28, or 29, wherein X₁₁ represents Arg (R).
57. The GLP-1-/GIP-/amylin-receptor tri-agonist according to any one of embodiments 7, 14, 15, 18, 24, 27, 28, or 29, wherein X₁₁ represents Gln (Q).
58. The GLP-1-/GIP-/amylin-receptor tri-agonist according to any one of embodiments 7, 14, 15, 27, 28 or 29, wherein X₁₁ represents Glu (E).
59. The GLP-1-/GIP-/amylin-receptor tri-agonist according to any one of embodiments 7, 14, 15, or 27, wherein X₁₁ represents Gly (G).
60. The GLP-1-/GIP-/amylin-receptor tri-agonist according to any one of embodiments 7, 14, 15, or 27, wherein X₁₁ represents His (H).
61. The GLP-1-/GIP-/amylin-receptor tri-agonist according to any one of embodiments 7, 14, 15, 18, 19, 26, or 27, wherein X₁, represents Lys (K).
62. The GLP-1-/GIP-/amylin-receptor tri-agonist according to any one of embodiments 7, 14, 15, or 27, wherein X₁₁ represents Thr (T).
63. The GLP-1-/GIP-/amylin-receptor tri-agonist according to any one of embodiments 7, 14, 15, or 27, wherein X₁₁ represents Tyr (Y).
64. The GLP-1-/GIP-/amylin-receptor tri-agonist according to any one of embodiments 7, 14, 15, 18, 19, 25, 26, 27, 28 or 29, wherein X₁₂ represents Ala (A).
65. The GLP-1-/GIP-/amylin-receptor tri-agonist according to any one of embodiments 7, 14, 15, 18, 24, 26, or 27, wherein X₁₂ represents Lys (K).
66. The GLP-1-/GIP-/amylin-receptor tri-agonist according to any one of embodiments 7, 14, 15, 27, or 28, wherein X₁₃ represents Asp (D).
67. The GLP-1-/GIP-/amylin-receptor tri-agonist according to any one of embodiments 7, 14, 15, 16, 18, 24, 25, 26, 27, 28 or 29, wherein X₁₃ represents Thr (T).
68. The GLP-1-/GIP-/amylin-receptor tri-agonist according to any one of embodiments 7, 14, 15, 18, 19, 24, 26, 27, 28 or 29, wherein X₁₄ represents Arg (R).
69. The GLP-1-/GIP-/amylin-receptor tri-agonist according to any one of embodiments 7, 14, 15, 18, 25, 26 or 27, wherein X₁₄ represents Lys (K).
70. The GLP-1-/GIP-/amylin-receptor tri-agonist according to any one of embodiments 7, 14, 15, 18, 19 or 27, wherein X₁₅ represents Ala (A).
71. The GLP-1-/GIP-/amylin-receptor tri-agonist according to any one of embodiments 7, 14, 15, 18, 19, 23, 24, 25, 26, 27, 28 or 29 wherein X₁₅ represents Ser (S).
72. The GLP-1-/GIP-/amylin-receptor tri-agonist according to embodiment 8,
   wherein the peptide Z1 comprises or consists of the amino acid sequence according to Formula Xa SEQ ID NO: 162):

(Xa)
YX₅₁EGTFTSDYSX₅₂LLEEIAAX₅₃EFIX₅₄WLX₅₅X₅₆GGPSSX₅₇,

wherein
X₅₁ represents Aib,
X₅₂ represents Ile (I) or Lys (K),
X₅₃ represents Arg (R),
X₅₄ represents Glu (E),
X₅₅ represents Leu (L) or I (Ile),
X₅₆ represents Ala (A),
X₅₇ represents Gly (G) or Glu (E).
73. The GLP-1-/GIP-/amylin-receptor tri-agonist according to embodiment 8 or embodiment 72, wherein the peptide Z2 comprises or consists of the amino acid sequence according to Formula XIIa (SEQ ID NO: 165):

(XIIa)
AX₅₈X₅₉LSTAX₆₀X₆₁X₆₂RLSAELHX₆₃LATX₆₄PRTETGSGSP,

wherein
X₅₈ represents Ser (S),
X₅₉ represents His (H),
X₆₀ represents Gln (Q),
X₆₁ represents Thr (T),
X₆₂ represents Gln (Q),
X₆₃ represents Lys (K),
X₆₄ represents Leu (L).
74. The GLP-1-/GIP-/amylin-receptor tri-agonist according to any one of the embodiments 8, 72 or 73,
   wherein the peptide Z1 comprises or consists of the amino acid sequence according to Formula Xa (SEQ ID NO: 162):

(Xa)
YX₅₁EGTFTSDYSX₅₂LLEEIAAX₅₃EFIX₅₄WLX₅₅X₅₆GGPSSX₅₇,

wherein
   X₅₁ represents Aib,
   X₅₂ represents Ile (I) or Lys (K),
   X₅₃ represents Arg (R),
   X₅₄ represents Glu (E),
   X₅₅ represents Leu (L) or I (Ile),
   X₅₆ represents Ala (A),
   X₅₇ represents Gly (G) or Glu (E); and
wherein the peptide Z2 comprises or consists of the amino acid sequence according to Formula XIIa (SEQ ID NO: 165):

(XIIa)
AX₅₈X₅₉LSTAX₆₀X₆₁X₆₂RLSAELHX₆₃LATX₆₄PRTETGSGSP,

wherein
X₅₈ represents Ser (S),
X₅₉ represents His (H),
X₆₀ represents Gln (Q),
X₆₁ represents Thr (T),
X₆₂ represents Gln (Q),
X₆₃ represents Lys (K),
X₆₄ represents Leu (L).
75. The GLP-1-/GIP-/amylin-receptor tri-agonist according to any one of the embodiments 8 or 72 to 74,
   wherein the peptide Z1 comprises or consists of the amino acid sequence according to Formula Xa (SEQ ID NO: 162):

(Xa)
YX₅₁EGTFTSDYSX₅₂LLEEIAAX₅₃EFIX₅₄WLX₅₅X₅₆GGPSSX₅₇,

wherein
X₅₁ represents Aib,
X₅₂ represents Ile (I) or Lys (K),
X₅₃ represents Arg (R),
X₅₄ represents Glu (E),
X₅₅ represents Leu (L),
X₅₆ represents Ala (A),
X₅₇ represents Gly (G).
76. The GLP-1-/GIP-/amylin-receptor tri-agonist according to any one of the embodiments 8 or 72 to 75,
   wherein the peptide Z1 comprises or consists of the amino acid sequence according to Formula XI SEQ ID NO: 163):

(XI)
YX₅₁EGTFTSDYSX₅₂LLEEIAAREFIEWLLAGGPSSG,

wherein
X₅₁ represents Aib,
X₅₂ represents Ile (I) or Lys (K).
77. The GLP-1-/GIP-/amylin-receptor tri-agonist according to any one of the embodiments 8 or 72 to 77,
   wherein the peptide Z2 comprises or consists of the amino acid sequence according to Formula XIII SEQ ID NO: 166):

(XIII)
ASX₅₉LSTAQTQRLSAELHKLATLPRTETGSGSP,

wherein
X₅₉ represents Glu (E) or His (H).
78. The GLP-1-/GIP-/amylin-receptor tri-agonist according to any one of embodiments 1 to 7 and 9 to 13,
   wherein the peptide Z1-L1-Z2 comprises or consists of an amino acid sequence selected from the group consisting of SEQ ID NOs 170 to 242, wherein X represents Aib.
79. The GLP-1-/GIP-/amylin-receptor tri-agonist according to any one of the preceding embodiments wherein the backbone of the peptide Z1-L1-Z2 comprises 66 to 80 amino acid residues.
80. The GLP-1-/GIP-/amylin-receptor tri-agonist according to any one of the preceding embodiments wherein the backbone of the peptide Z1-L1-Z2 comprises 67, 68, 75 or 76 amino acid residues, preferably 76 amino acid residues.
81. The GLP-1-/GIP-/amylin-receptor tri-agonist according to any one of the preceding embodiments wherein the peptide is a peptide derivative comprising a protraction moiety.
82. The GLP-1-/GIP-/amylin-receptor tri-agonist according to any one of the preceding embodiments wherein the peptide is a peptide derivative comprising a protraction moiety, wherein said protraction moiety comprises a protractor P being a C₁₂-C₂₀ diacid.
83. The GLP-1-/GIP-/amylin-receptor tri-agonist according to any one of the preceding embodiments, wherein the peptide is a peptide derivative comprising a protraction moiety, wherein said protraction moiety comprise a protractor P selected from the group consisting of:

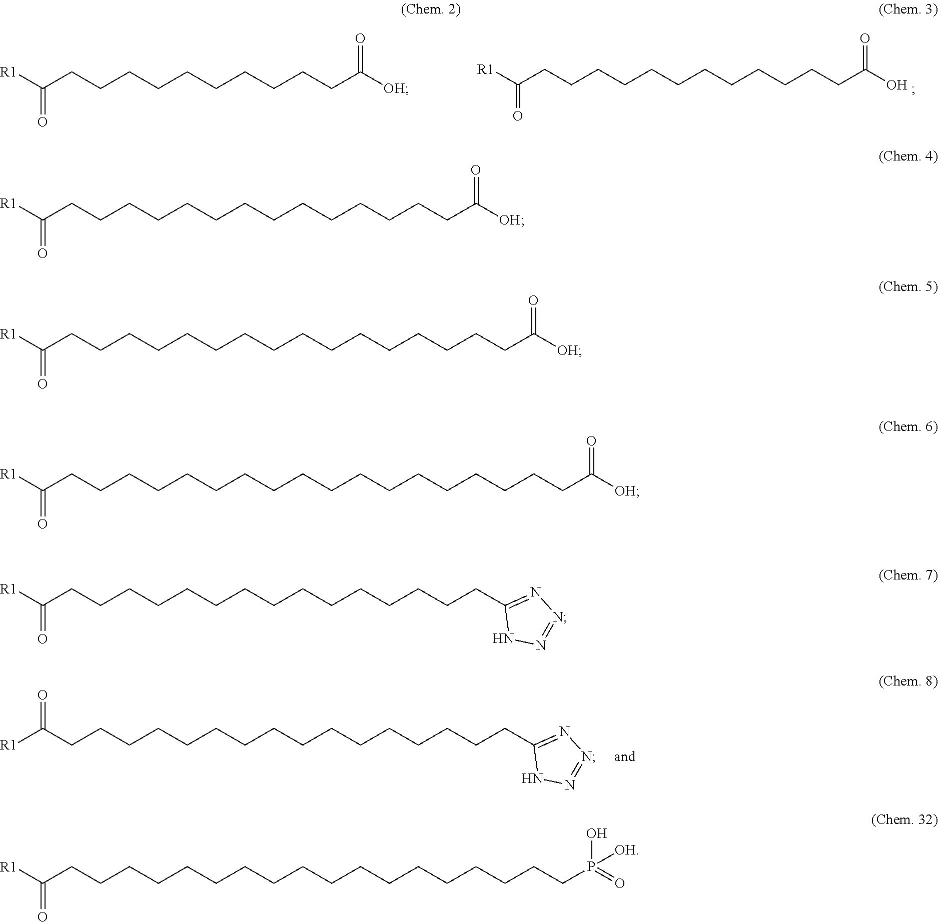

84. The GLP-1-/GIP-/amylin-receptor tri-agonist according to any one of the preceding embodiments, wherein the peptide is a peptide derivative comprising a protraction moiety, wherein said protraction moiety comprises a protractor P selected from the group consisting of C₁₆ diacid, C₁₈ diacid, C₂₀ diacid, and C₁₉ phosphonic acid; preferably the protractor P is a C₁₈ diacid or a C₂₀ diacid.
85. The GLP-1-/GIP-/amylin-receptor tri-agonist according to any one of the preceding embodiments, wherein the peptide is a peptide derivative comprising a protraction moiety, wherein the protraction moiety is attached to the epsilon position of the one lysine (Lys, K) residue.
86. The GLP-1-/GIP-/amylin-receptor tri-agonist according to any one of the preceding embodiments, wherein the peptide is a peptide derivative comprising a protraction moiety, wherein the protraction moiety is attached to the epsilon position of the lysine (Lys, K) residue in the peptide Z1 or to the epsilon position of the lysine (Lys, K) residue in the peptide Z2.
87. The GLP-1-/GIP-/amylin-receptor tri-agonist according to any one of the preceding embodiments, wherein the peptide is a peptide derivative comprising a protraction moiety, wherein the protraction moiety is attached to the epsilon position of the lysine (Lys, K) residue at position 12 or position 33 or position 34 of peptide Z1, preferably at position 12 or position 33 of peptide Z1.
88. The GLP-1-/GIP-/amylin-receptor tri-agonist according to any one of the preceding embodiments, wherein the peptide is a peptide derivative comprising a protraction moiety, wherein the protraction moiety is attached to the epsilon position of the lysine (Lys, K) residue at positions 3, 15, 18, 20, or 24 of peptide Z2.
89. The GLP-1-/GIP-/amylin-receptor tri-agonist according to any one of the preceding embodiments, wherein the peptide is a peptide derivative comprising a protraction moiety, wherein the protraction moiety is attached to the epsilon position of the lysine (Lys, K) residue at position 15 of peptide Z2 or at position 18 of peptide Z2.
90. The GLP-1-/GIP-/amylin-receptor tri-agonist according to any one of the preceding embodiments, wherein the peptide is a peptide derivative comprising a protraction moiety, wherein said protraction moiety further comprises a linker L_{P} selected from the group consisting of:

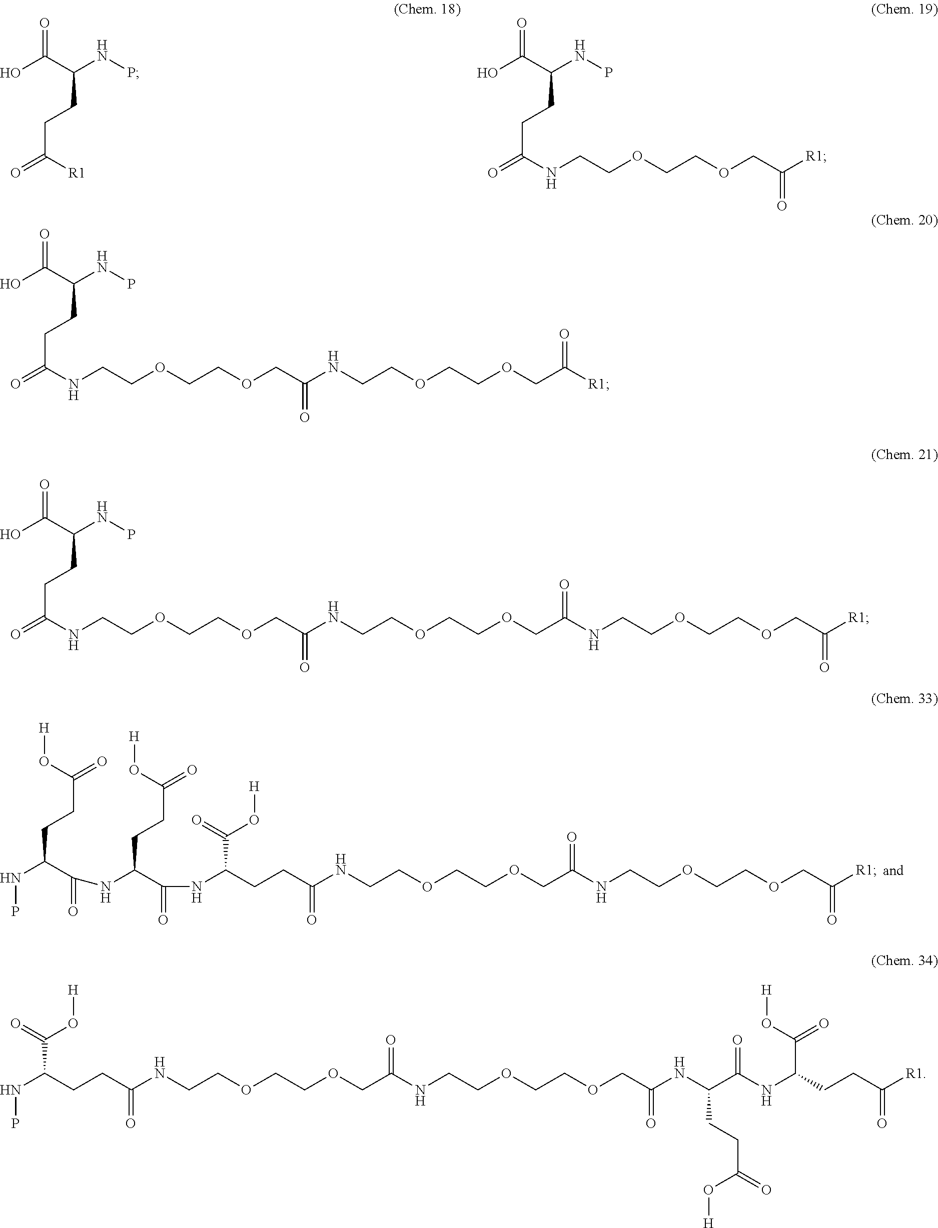

91. The GLP-1-/GIP-/amylin-receptor tri-agonist according to any one of the preceding embodiments, wherein the peptide is a peptide derivative comprising a protraction moiety, wherein said protraction moiety comprises Chem. 20 or Chem. 21 as linker L and Chem. 5 or Chem. 6 as protractor P.
92. The GLP-1-/GIP-/amylin-receptor tri-agonist according to any one of the preceding embodiments, wherein the peptide is a peptide derivative comprising a protraction moiety,
   wherein said protraction moiety consists of (i) a linker L_{P} selected from the group presented in Table 4 and (ii) a protractor P selected from the group presented in Table 3, preferably said protraction moiety is selected from the group presented in Table 5.
93. The GLP-1-/GIP-/amylin-receptor tri-agonist according to any one of the preceding embodiments, wherein the peptide is a peptide derivative comprising a protraction moiety, wherein said protraction moiety is a C₁₈ diacid (S) gamma-Glu 2×Ado fatty acid moiety (Chem. 28) or a C₂₀ diacid (S) gamma-Glu 2×Ado fatty acid moiety (Chem. 27).
94. The GLP-1-/GIP-/amylin-receptor tri-agonist according to any one of the preceding embodiments,
   wherein the peptide Z1 comprises or consists of the amino acid sequence according to Formula IIa (SEQ ID NO: 3):

(IIa)
X₁X₂X₃GTFTSDYSILLEEQAAREFIEWLLAGGPSX₄X₅,

wherein
   X₁ represents His (H) or Tyr (Y),
   X₂ represents Aib,
   X₃ represents Glu (E) or His (H),
   X₄ represents Arg (R), Gly (G) or Ser (S),
   X₅ represents Gly (G);
wherein the peptide linker L1 comprises or consists of the amino acid sequence according to Formula IV:

(IV)
X₁X₂X₃X₄X₅X₆X₇X₈X₉X₁₀X₁₁X₁₂X₁₃X₁₄,

which is selected from the group consisting of E, GE, APPPSGGGE (SEQ ID NO: 129), AGQAPGQAPG (SEQ ID NO: 136) and AGQAPGQAPL (SEQ ID NO: 137);
wherein the peptide Z2 comprises or consists of the amino acid sequence according to Formula IIIa (SEQ ID NO: 4):

(IIIa)
ASX₆LSTAX₇X₈X₉RLSAX₁₀LHX₁₁LX₁₂X₁₃LPX₁₄TETGSGX₁₅P,

wherein
   X₆ represents Gln (Q), Glu (E), His (H) or Lys (K),
   X₇ represents Ala (A) or Gln (Q),
   X₈ represents Gln (Q), Leu (L) or Thr (T),
   X₉ represents Ala (A), Gly (G) or Gln (Q),
   X₁₀ represents Glu (E) or Lys (K),
   X₁₁ represents Arg (R), Gln (Q), Glu (E), Gly (G), His (H), Lys (K), Thr (T) or Tyr (Y),
   X₁₂ represents Ala (A) or Lys (K),
   X₁₃ represents Asp (D) or Thr (T),
   X₁₄ represents Arg (R) or Lys (K),
   X₁₅ represents Ala (A) or Ser (S); and
wherein the peptide is a peptide derivative comprising a protraction moiety.
95. The GLP-1-/GIP-/amylin-receptor tri-agonist according to embodiment 94, wherein the peptide is a peptide derivative comprising a protraction moiety, wherein said protraction moiety comprises a protractor P being a C₁₂-C₂₀ diacid.
96. The GLP-1-/GIP-/amylin-receptor tri-agonist according to embodiment 95, wherein the peptide is a peptide derivative comprising a protraction moiety, wherein said protraction moiety further comprises a linker L_{P} selected from the group presented in Table 4.
97. The GLP-1-/GIP-/amylin-receptor tri-agonist according to any one of the embodiments 94 to 96, wherein the peptide Z2 comprises or consists of the amino acid sequence according to Formula IIIa (SEQ ID NO: 4):

(IIIa)
ASX₆LSTAX₇X₈X₉RLSAX₁₀LHX₁₁LX₁₂X₁₃LPX₁₄TETGSGX₁₅P,

wherein
   X₆ represents Glu (E) or His (H),
   X₇ represents Ala (A) or Gln (Q),
   X₈ represents Gln (Q), Leu (L) or Thr (T),
   X₉ represents Ala (A), Gly (G) or Gln (Q),
   X₁₀ represents Glu (E),
   X₁₁ represents Lys (K),
   X₁₂ represents Ala (A),
   X₁₃ represents Thr (T),
   X₁₄ represents Arg (R),
   X₁₅ represents Ala (A) or Ser (S).
98. The GLP-1-/GIP-/amylin-receptor tri-agonist according to any one of the embodiments 94 to 97, wherein the peptide Z2 comprises or consists of the amino acid sequence according to Formula VIII (SEQ ID NO: 9):

(VIII)
ASHLSTAQTQRLSAELHKLATLPRTETGSGSP.

99. The GLP-1-/GIP-/amylin-receptor tri-agonist according to any one of the embodiments 94 to 98, wherein the peptide Z2 comprises or consists of the amino acid sequence according to Formula VII (SEQ ID NO: 8):

(VII)
ASHLSTAQTQRLSAKLHRLATLPRTETGSGSP.

100. The GLP-1-/GIP-/amylin-receptor tri-agonist according to any one of the embodiments 1 to 93,
   wherein the peptide Z1 comprises or consists of the amino acid sequence according to Formula Xa (SEQ ID NO: 162):

(Xa)
YX₅₁EGTFTSDYSX₅₂LLEEIAAX₅₃EFIX₅₄WLX₅₅X₅₆GGPSSX₅₇,

wherein
   X₅₁ represents Aib,
   X₅₂ represents Ile (I) or Lys (K),
   X₅₃ represents Arg (R) or Gln (Q),
   X₅₄ represents Ala (A), Glu (E) or Gln (Q),
   X₅₅ represents Leu (L) or I (Ile),
   X₅₆ represents Ala (A) or Gln (Q),
   X₅₇ represents Gly (G) or Glu (E);
wherein the peptide linker L1 comprises or consists of the amino acid sequence according to Formula IV:

(IV)
X₁X₂X₃X₄X₅X₆X₇X₈X₉X₁₀X₁₁X₁₂X₁₃X₁₄,

which is selected from the group consisting of E, AG, AGQAPGQAPG (SEQ ID NO: 136), AGQAPGQAPL (SEQ ID NO: 137), AGGGG (SEQ ID NO: 142), AGEAPGQAPG (SEQ ID NO: 143), and AGQAPGEAPG (SEQ ID NO: 154); wherein the peptide Z2 comprises or consists of the amino acid sequence according to Formula XIIa (SEQ ID NO: 165):

(XIIa)
AX₅₈X₅₉LSTAX₆₀X₆₁X₆₂RLSAELHX₆₃LATX₆₄PRTETGSGSP,

wherein
   X₅₈ represents Gly (G) or Ser (S),
   X₅₉ represents Gln (Q), Glu (E), or His (H),
   X₆₀ represents Ala (A) or Gln (Q),
   X₆₁ represents Leu (L) or Thr (T),
   X₆₂ represents Ala (A), Gly (G) or Gln (Q),
   X₆₃ represents Gln (Q), Glu (E), or Lys (K),
   X₆₄ represents Leu (L) or Glu (E); and
wherein the peptide is a peptide derivative comprising a protraction moiety.
101. The GLP-1-/GIP-/amylin-receptor tri-agonist according to embodiment 100, wherein the peptide is a peptide derivative comprising a protraction moiety, wherein said protraction moiety comprises a protractor P being a C₁₂-C₂₀ diacid.
102. The GLP-1-/GIP-/amylin-receptor tri-agonist according to embodiment 101, wherein the peptide is a peptide derivative comprising a protraction moiety, wherein said protraction moiety further comprises a linker L_{P} selected from the group presented in Table 4.
103. The GLP-1-/GIP-/amylin-receptor tri-agonist according to any one of the embodiments 100 to 102, wherein the peptide Z1 comprises or consists of the amino acid sequence according to Formula XI (SEQ ID NO: 163):

(XI)
YX₅₁EGTFTSDYSX₅₂LLEEIAAREFIEWLLAGGPSSG,

wherein
   X₅₁ represents Aib,
   X₅₂ represents Ile (I) or Lys (K).
104. The GLP-1-/GIP-/amylin-receptor tri-agonist according to any one of the embodiments 100 to 103, wherein the peptide Z2 comprises or consists of the amino acid sequence according to Formula XIII (SEQ ID NO: 166):

(XIII)
ASX₅₉LSTAQTQRLSAELHKLATLPRTETGSGSP,

wherein
   X₅₉ represents Glu (E) or His (H).
105. A GLP-1-/GIP-/amylin-receptor tri-agonist comprising a peptide according to Formula I:

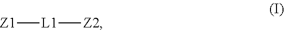

comprising one lysine (Lys, K) residue; wherein:
   Z1 is a peptide having a maximum of 4 amino acid substitutions relative to Formula II (SEQ ID NO: 1):

(II)
YX₂EGTFTSDYSILLEEQAAREFIEWLLAGGPSKG,

wherein the amino acid at position X₂ represents Aib, and wherein
Z1 comprises or consists of an amino acid sequence according to Formula IIa (SEQ ID NO: 3):

(IIa)
X₁X₂X₃GTFTSDYSILLEEQAAREFIEWLLAGGPSX₄X₅,

wherein
X₁ represents His (H) or Tyr (Y),
X₂ represents Aib,
X₃ represents Glu (E) or His (H),
X₄ represents Arg (R), Gly (G), Lys (K) or Ser (S),
X₅ represents Gly (G) or Lys (K);
L1 is a peptide linker comprising or consisting of 1 to 14, 1 to 10, 4 to 10 or 9 to 10 amino acid residues; and
Z2 is a peptide comprising a C-terminal amide, and having a maximum of 10 amino acid substitutions relative to Formula III (SEQ ID NO: 2):

(III)
ASELSTAALGRLSAELHELATLPRTETGSGSP,

and
Z2 comprises or consists of an amino acid sequence according to Formula Illa (SEQ ID NO: 4):

(IIIa)
ASX₆LSTAX₇X₈X₉RLSAX₁₀LHX₁₁LX₁₂X₁₃LPX₁₄TETGSGX₁₅P,

wherein
X₆ represents Gln (Q), Glu (E), His (H) or Lys (K),
X₇ represents Ala (A) or Gln (Q),
X₈ represents Gln (Q), Leu (L) or Thr (T),
X₉ represents Ala (A), Gly (G) or Gln (Q),
X₁₀ represents Glu (E) or Lys (K),
X₁₁ represents Arg (R), Gln (Q), Glu (E), Gly (G), His (H), Lys (K), Thr (T) or Tyr (Y),
X₁₂ represents Ala (A) or Lys (K),
X₁₃ represents Asp (D) or Thr (T),
X₁₄ represents Arg (R) or Lys (K),
X₁₅ represents Ala (A) or Ser (S);
wherein the peptide is a peptide derivative comprising a protraction moiety, wherein said protraction moiety comprises a protractor P being a C₁₆-C₂₀ diacid and further comprises a linker L_{P} selected from the group presented in Table 4.
106. A GLP-1-/GIP-/amylin-receptor tri-agonist comprising a peptide according to Formula I:

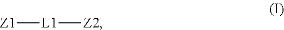

comprising one lysine (Lys, K) residue; wherein:
   Z1 is a peptide having a maximum of 4 amino acid substitutions relative to Formula II (SEQ ID NO: 1):

(II)
YX₂EGTFTSDYSILLEEQAAREFIEWLLAGGPSKG,

wherein the amino acid at position X₂ represents Aib, and wherein
Z1 comprises or consists of an amino acid sequence according to Formula Xa (SEQ ID NO: 162):

(Xa)
YX₅₁EGTFTSDYSX₅₂LLEEIAAX₅₃EFIX₅₄WLX₅₅X₅₆GGPSSX₅₇,

wherein
X₅₁ represents Aib,
X₅₂ represents Ile (I) or Lys (K),
X₅₃ represents Arg (R) or Gln (Q),
X₅₄ represents Ala (A), Glu (E) or Gln (Q),
X₅₅ represents Leu (L) or I (Ile),
X₅₆ represents Ala (A) or Gln (Q),
X₅₇ represents Gly (G) or Glu (E);
   L1 is a peptide linker comprising or consisting of 1 to 14, 1 to 10, 4 to 10 or 9 to 10 amino acid residues; and
   Z2 is a peptide comprising a C-terminal amide, and having a maximum of 10 amino acid substitutions relative to Formula III (SEQ ID NO: 2):

(III)
ASELSTAALGRLSAELHELATLPRTETGSGSP,

and
Z2 comprises or consists of an amino acid sequence according to Formula XIIa (SEQ ID NO: 165):

(XIIa)
AX₅₈X₅₉LSTAX₆₀X₆₁X₆₂RLSAELHX₆₃LATX₆₄PRTETGSGSP,

wherein
X₅₈ represents Gly (G) or Ser (S),
X₅₉ represents Gln (Q), Glu (E), or His (H),
X₆₀ represents Ala (A) or Gln (Q),
X₆₁ represents Leu (L) or Thr (T),
X₆₂ represents Ala (A), Gly (G) or Gln (Q),
X₆₃ represents Gln (Q), Glu (E), or Lys (K),
X₆₄ represents Leu (L) or Glu (E);
wherein the peptide is a peptide derivative comprising a protraction moiety,
wherein said protraction moiety comprises a protractor P being a C₁₆-C₂₀ diacid and further comprises a linker L_{P} selected from the group presented in Table 4.
107. The GLP-1-/GIP-/amylin-receptor tri-agonist according to any one of the preceding embodiments selected from:

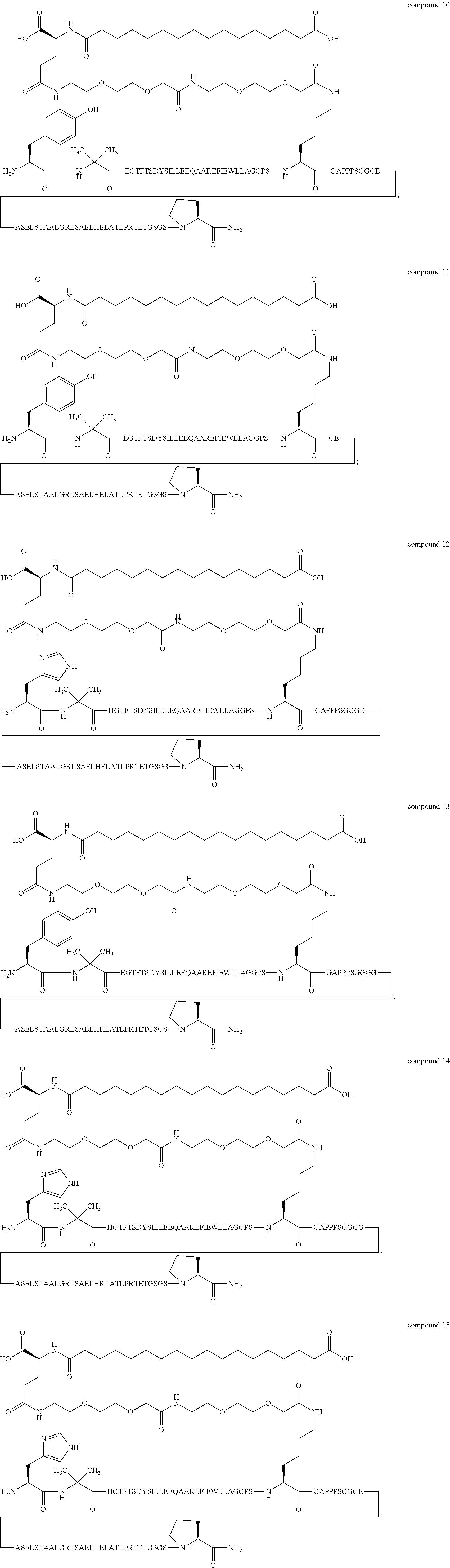

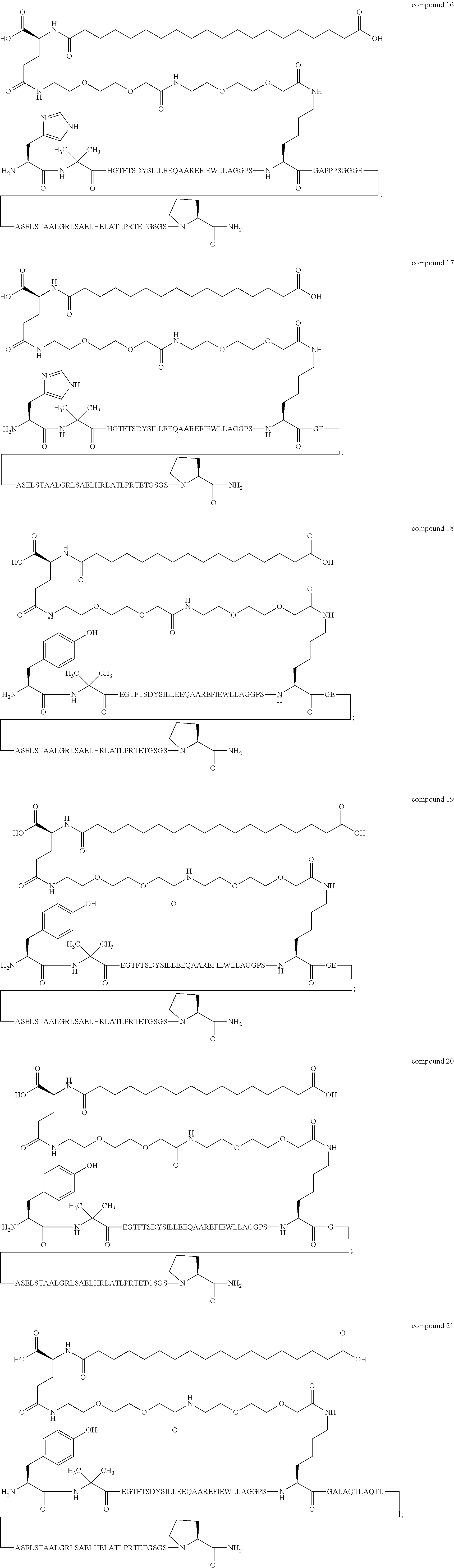

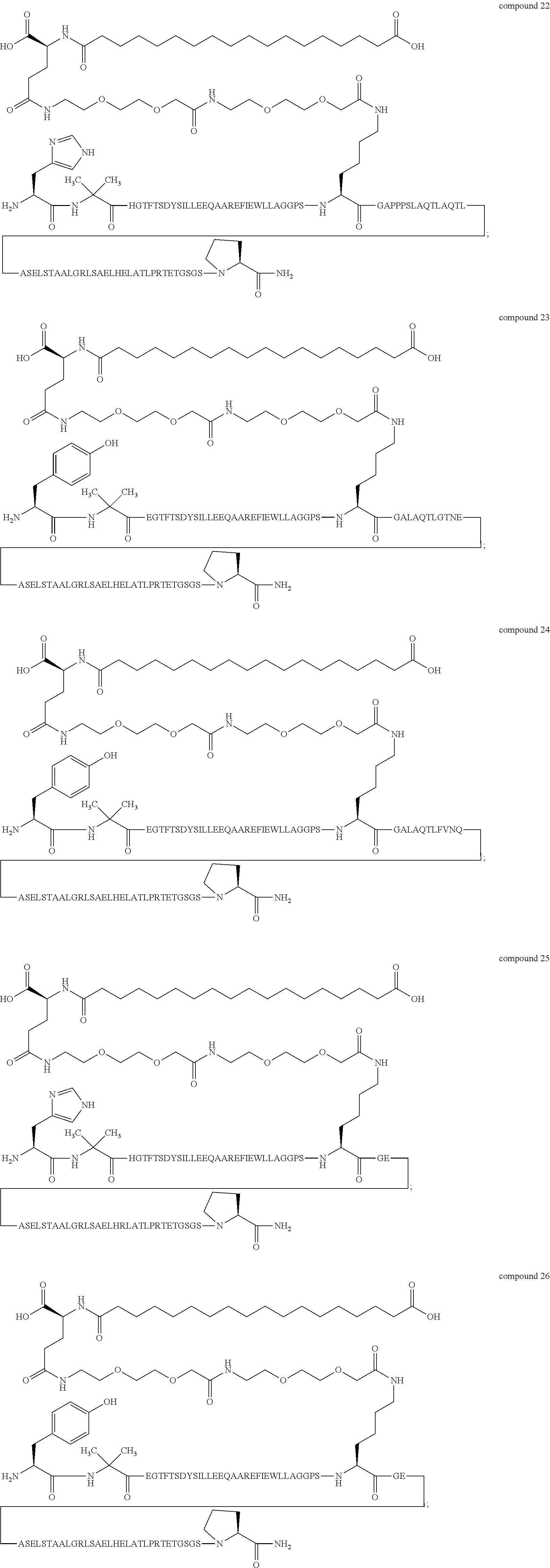

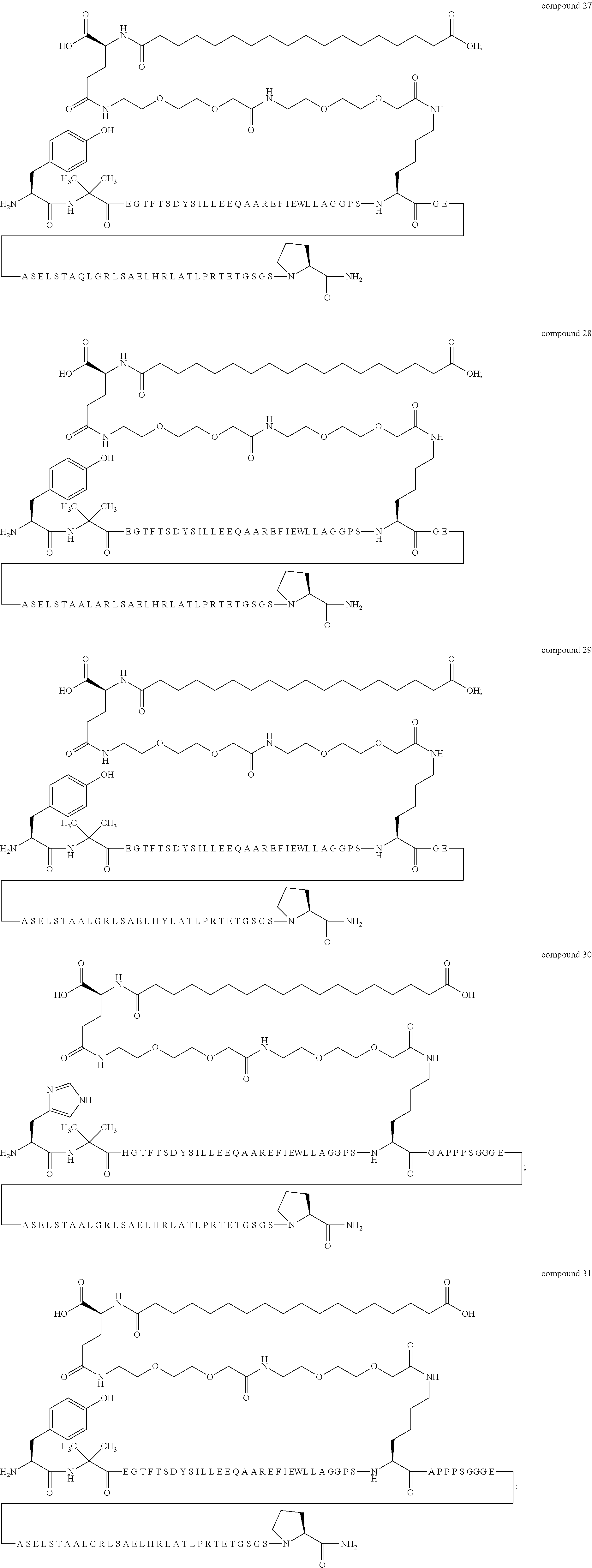

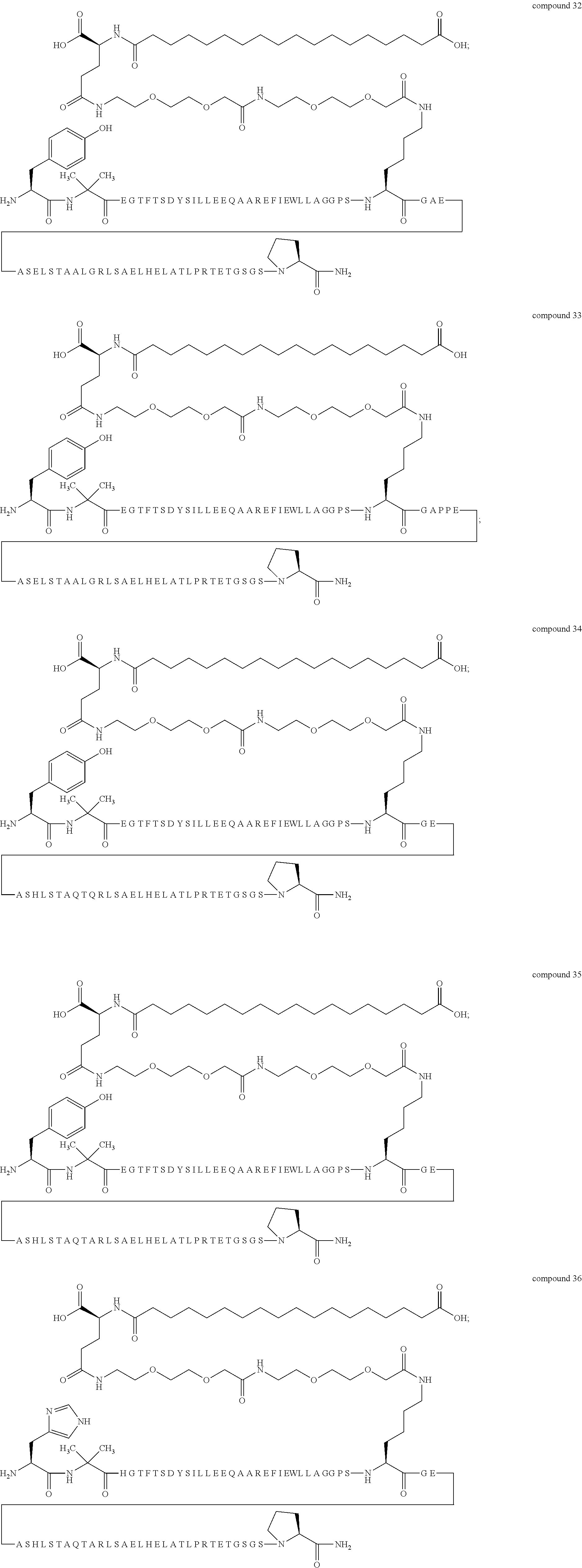

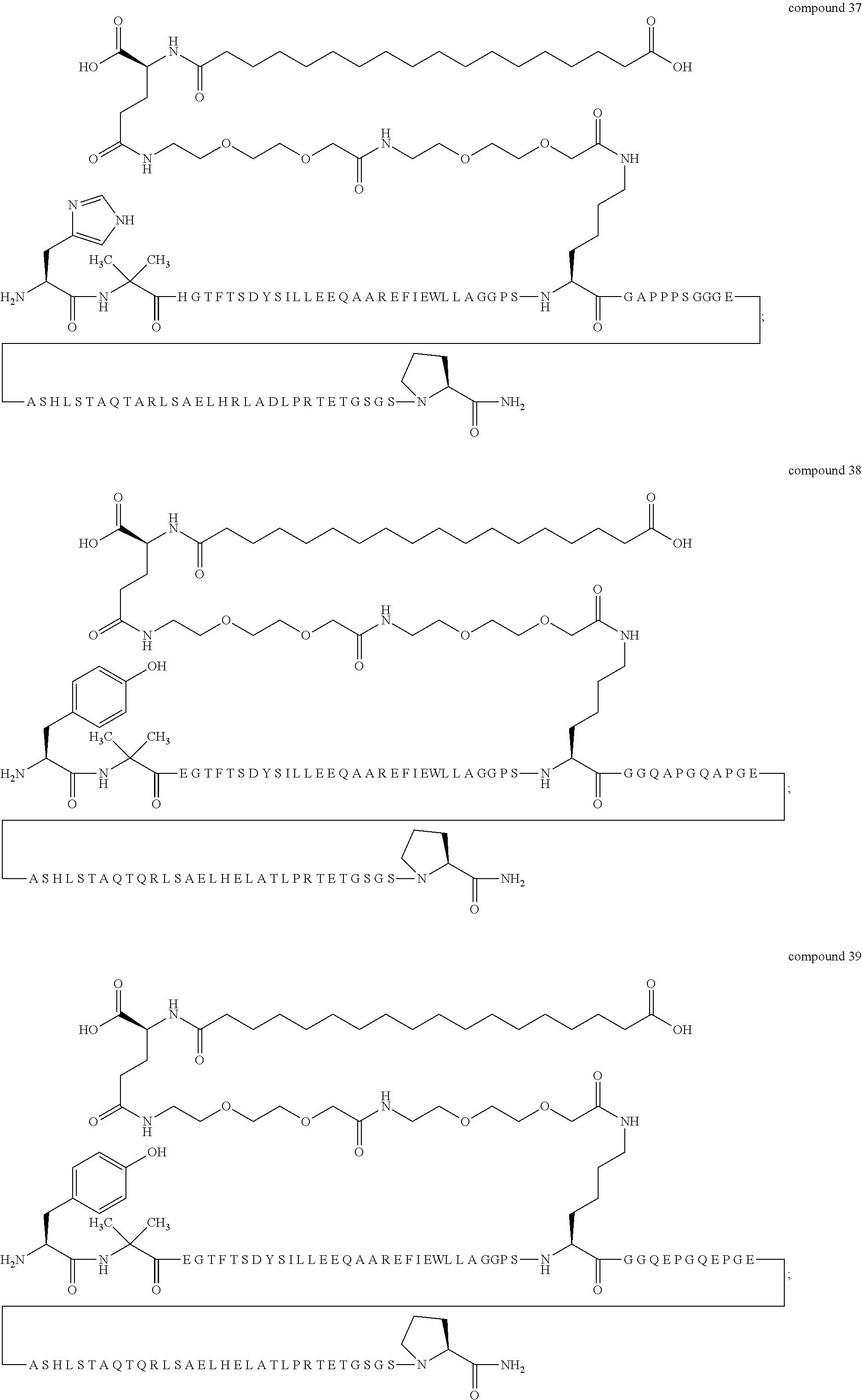

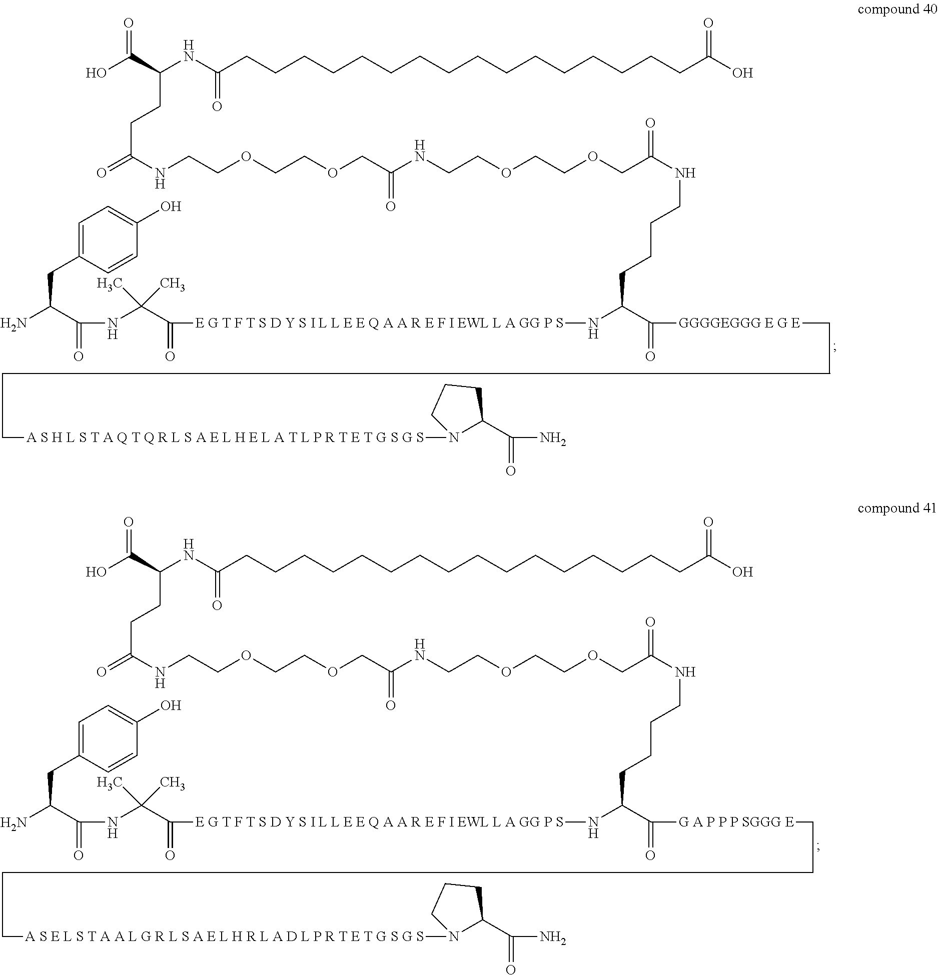

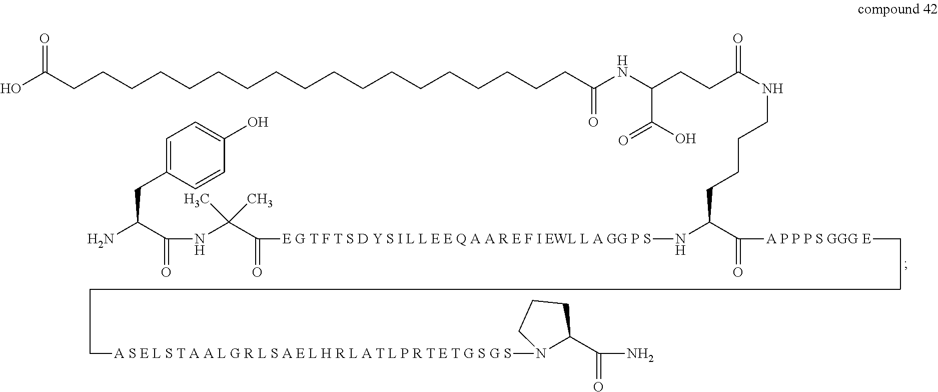

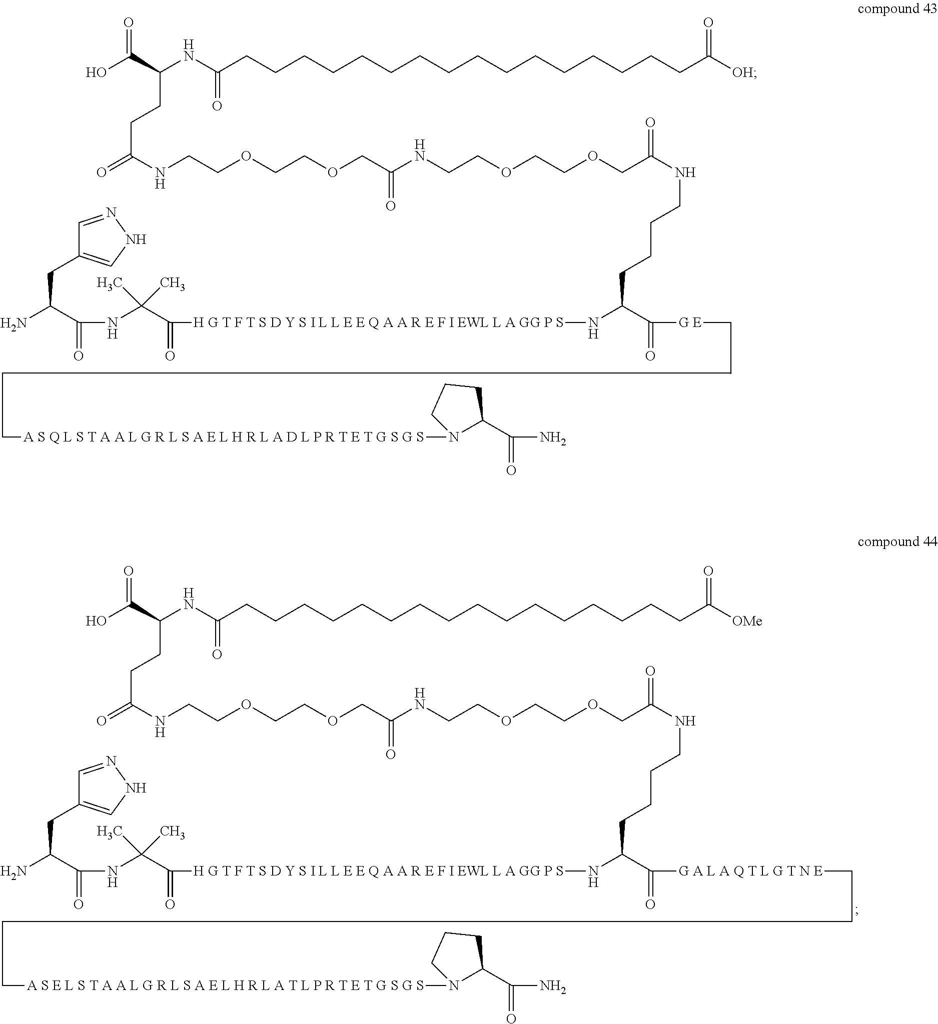

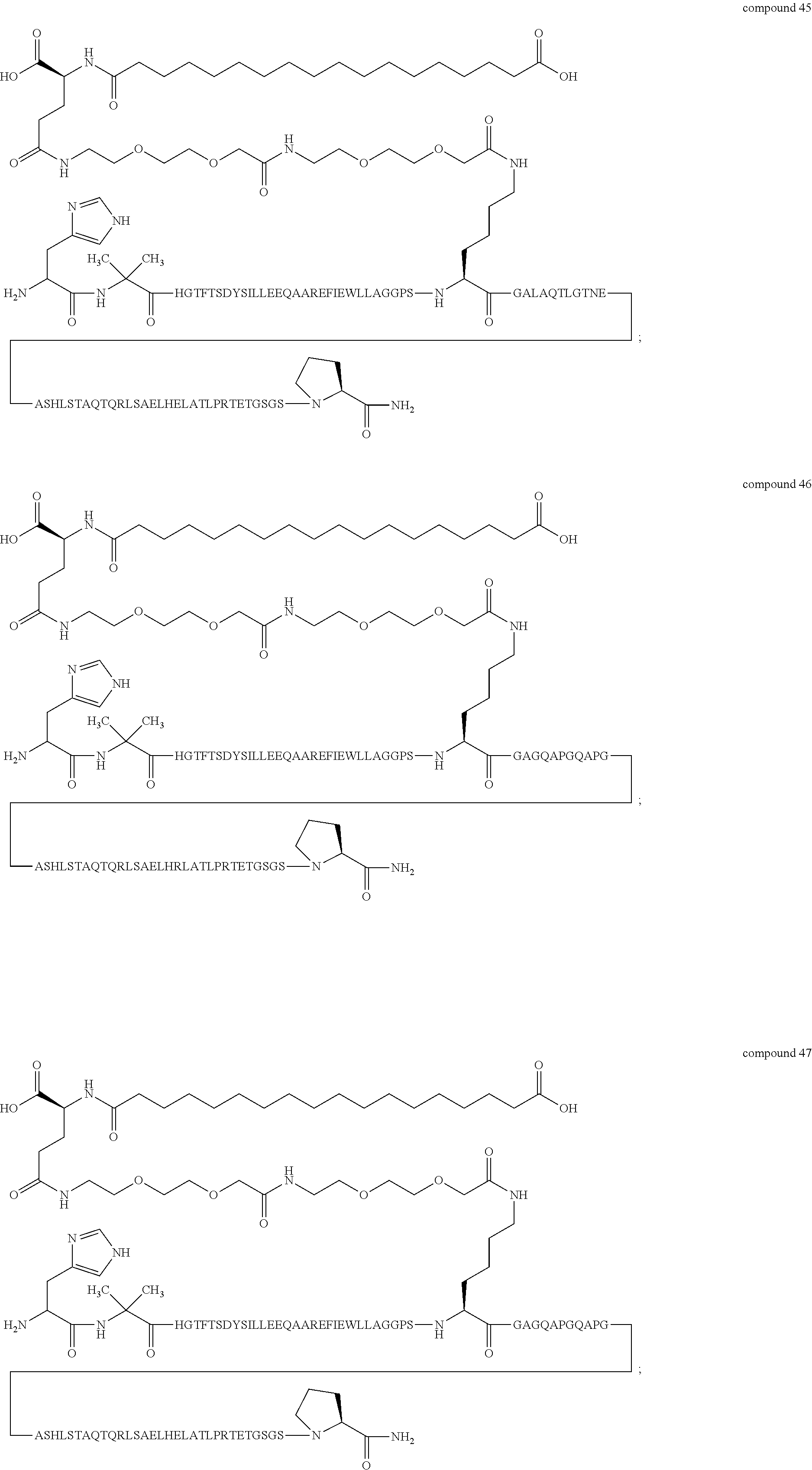

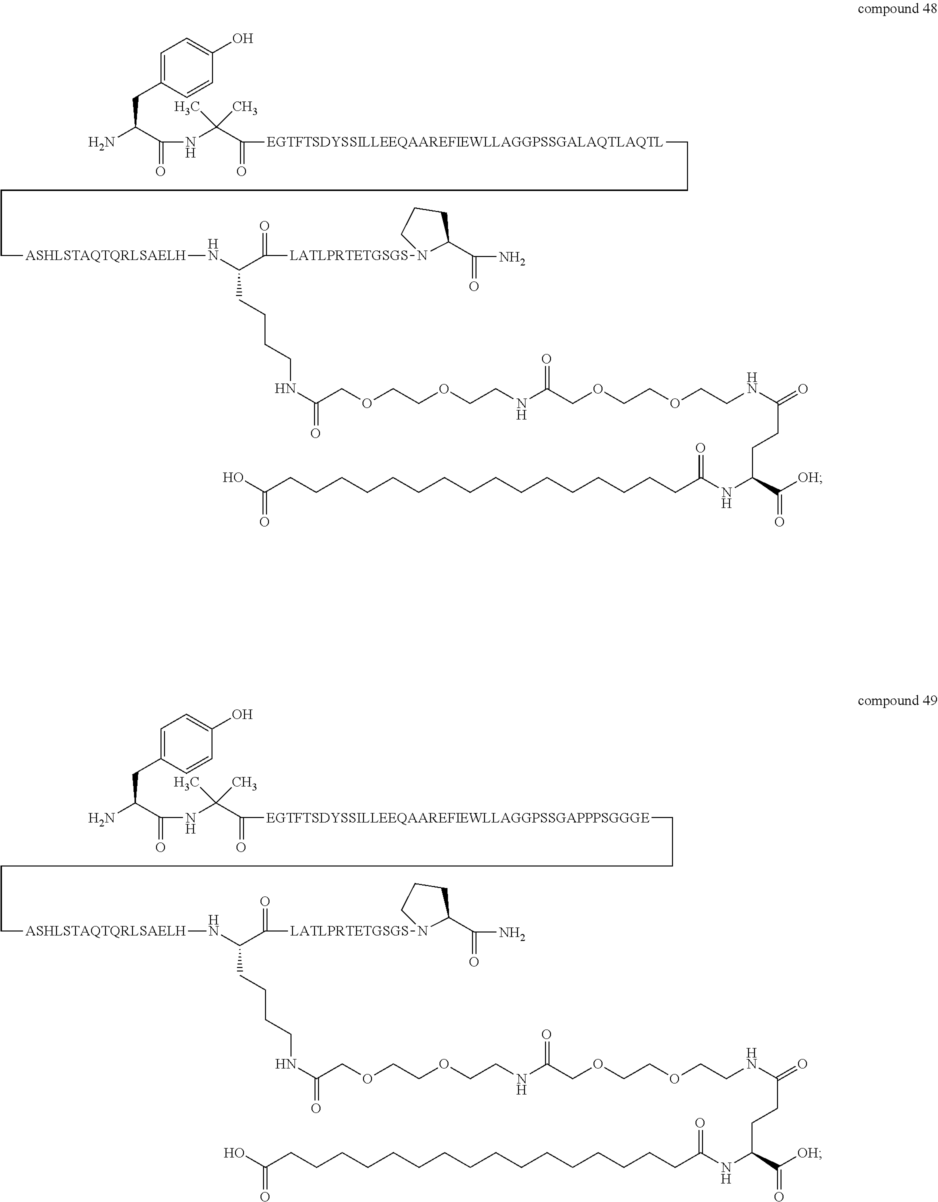

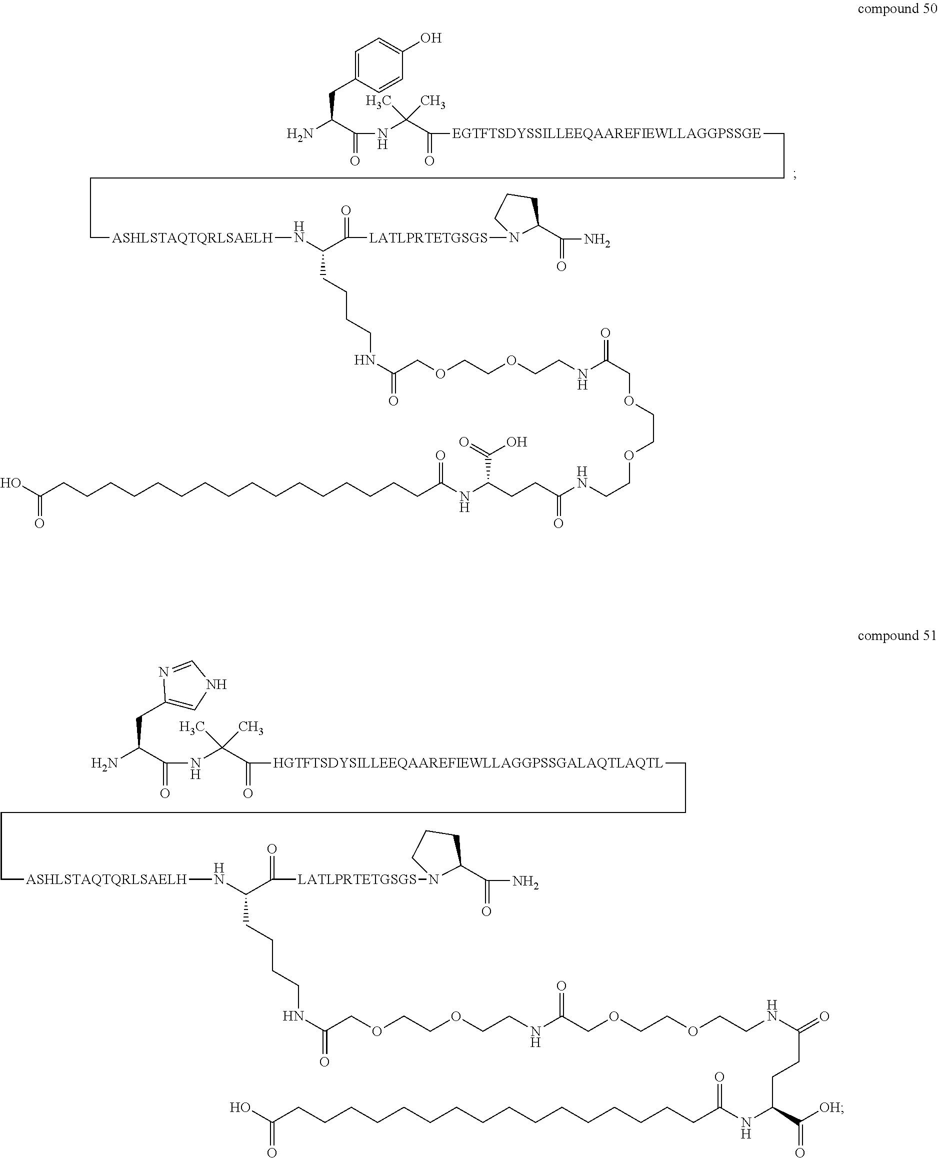

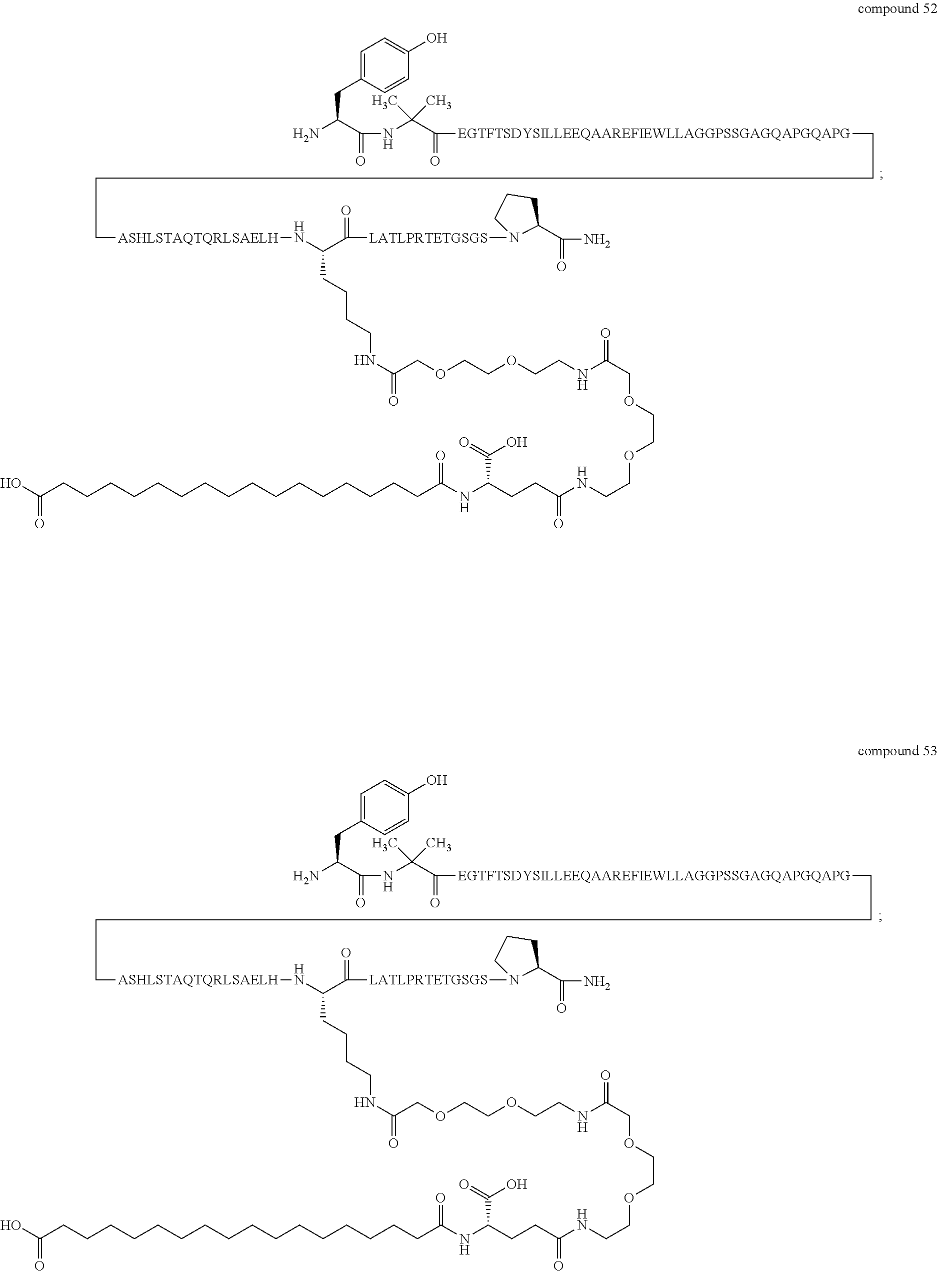

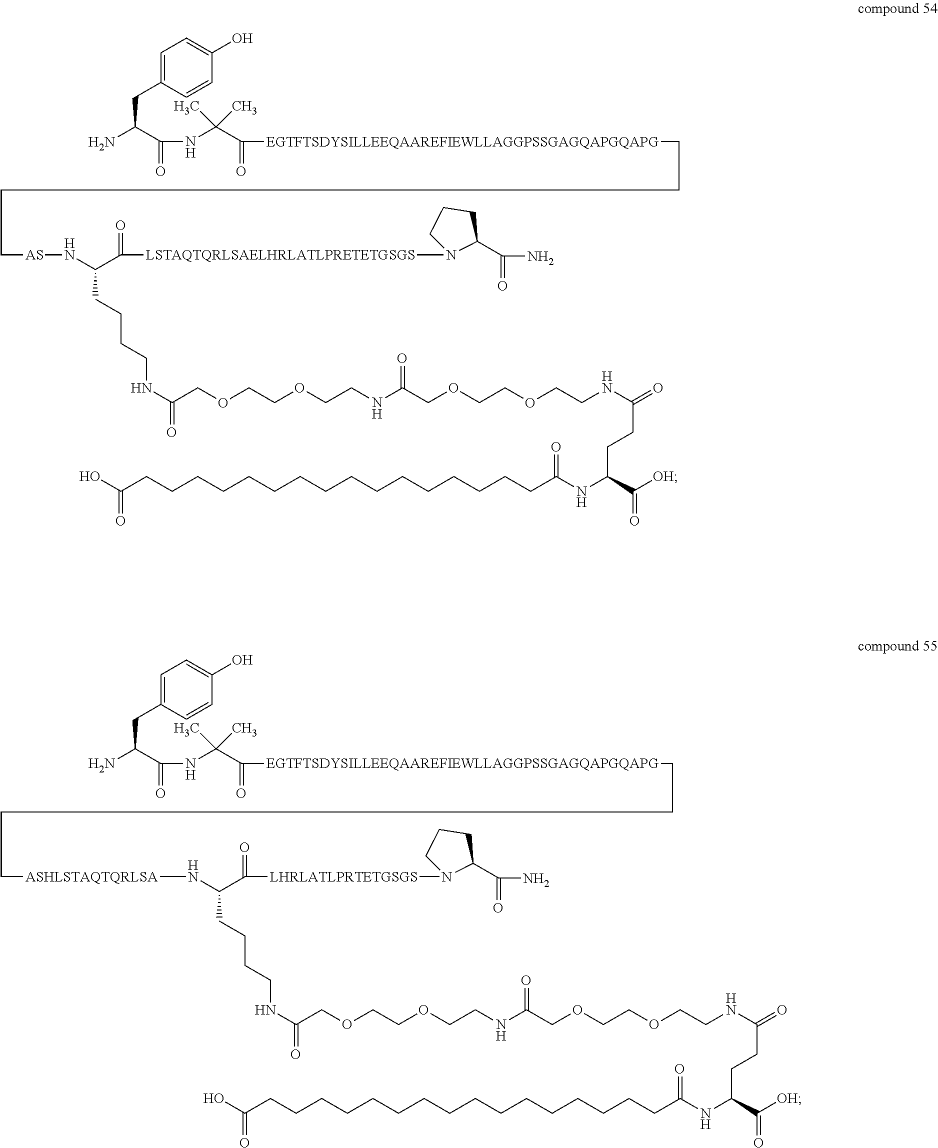

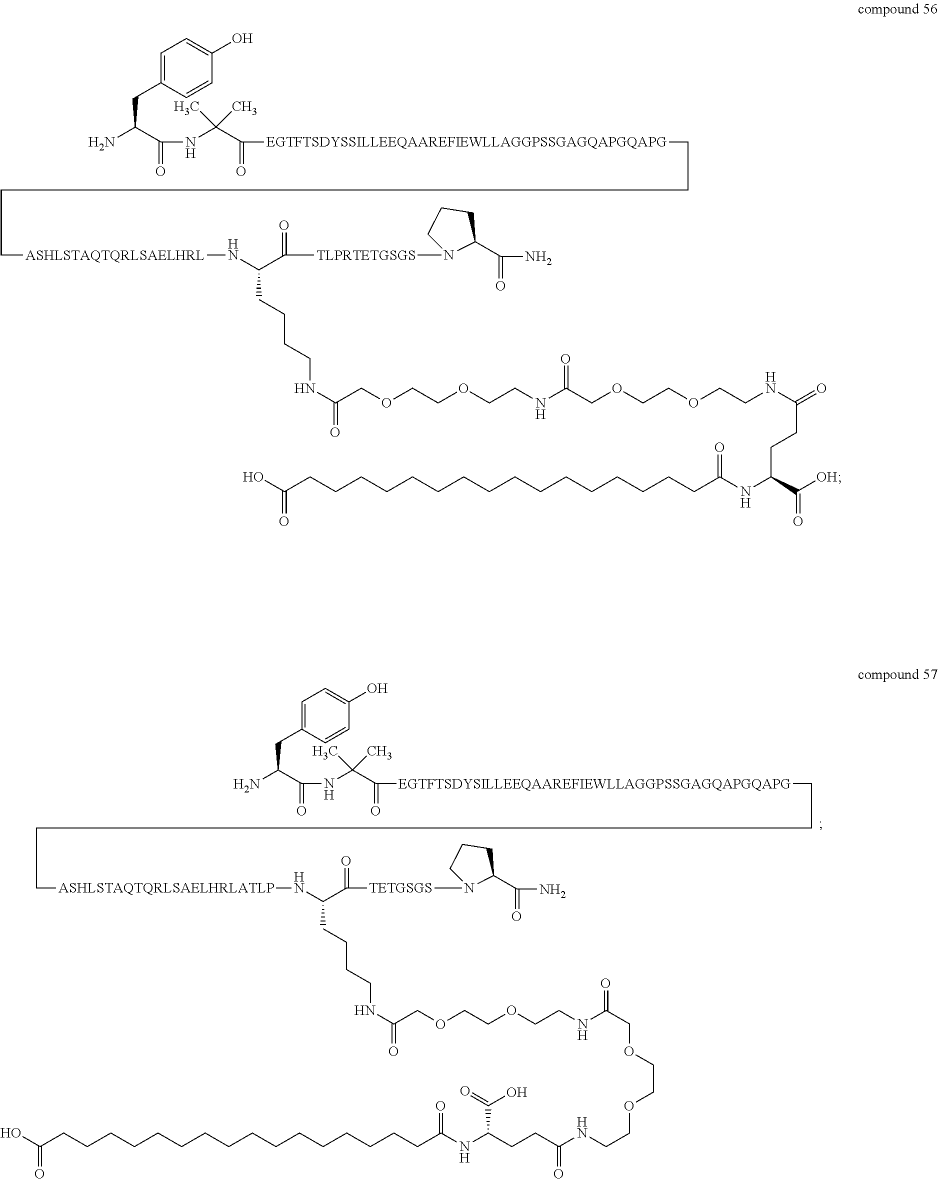

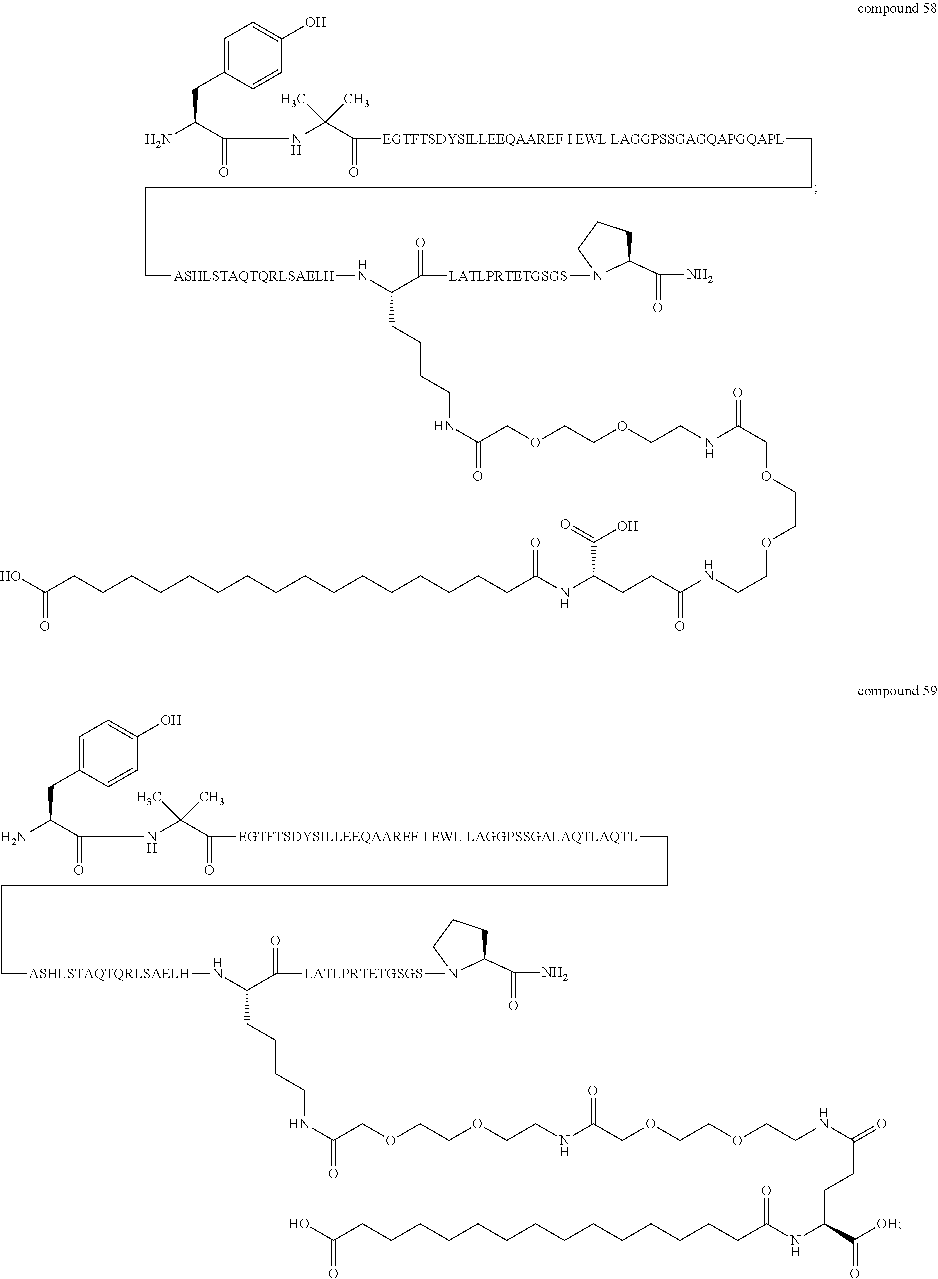

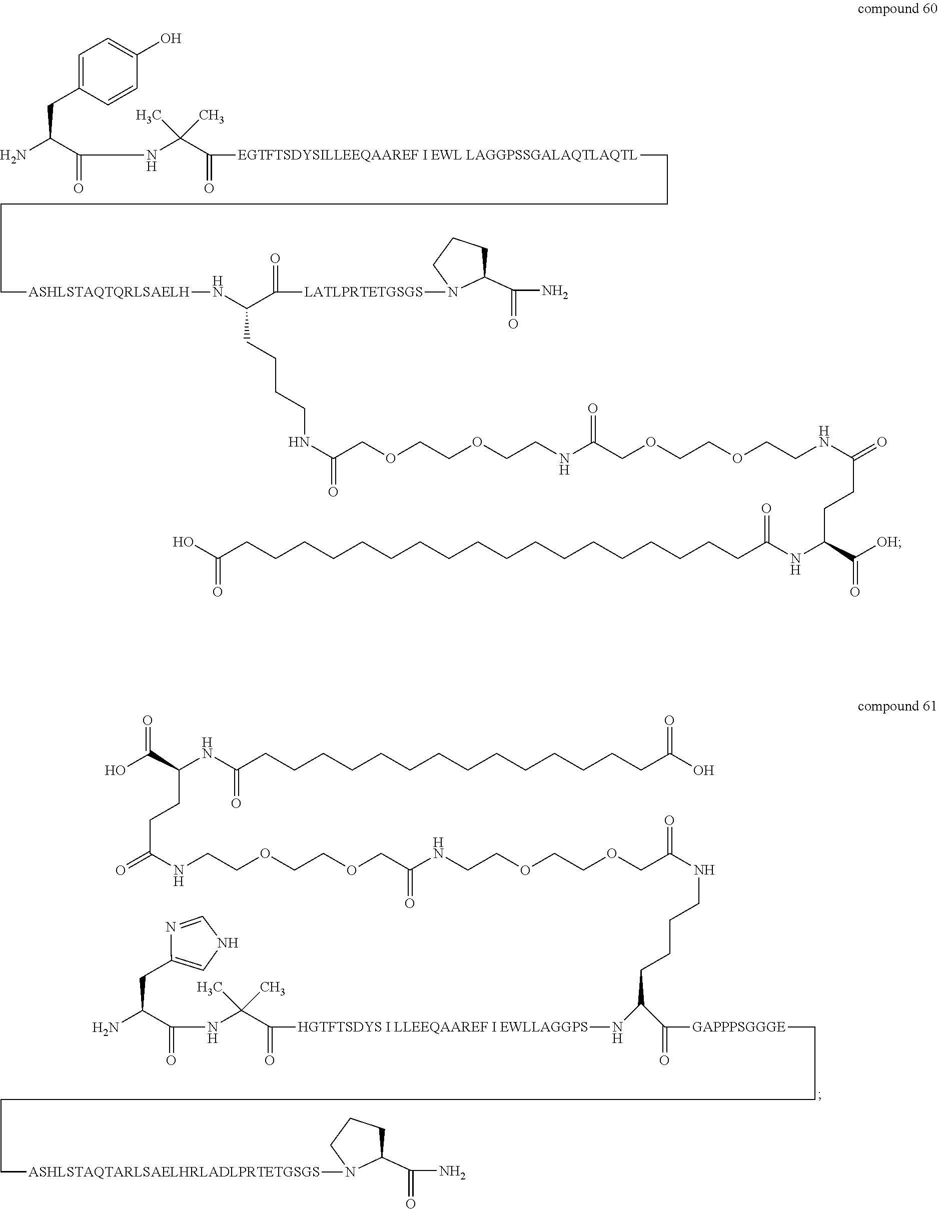

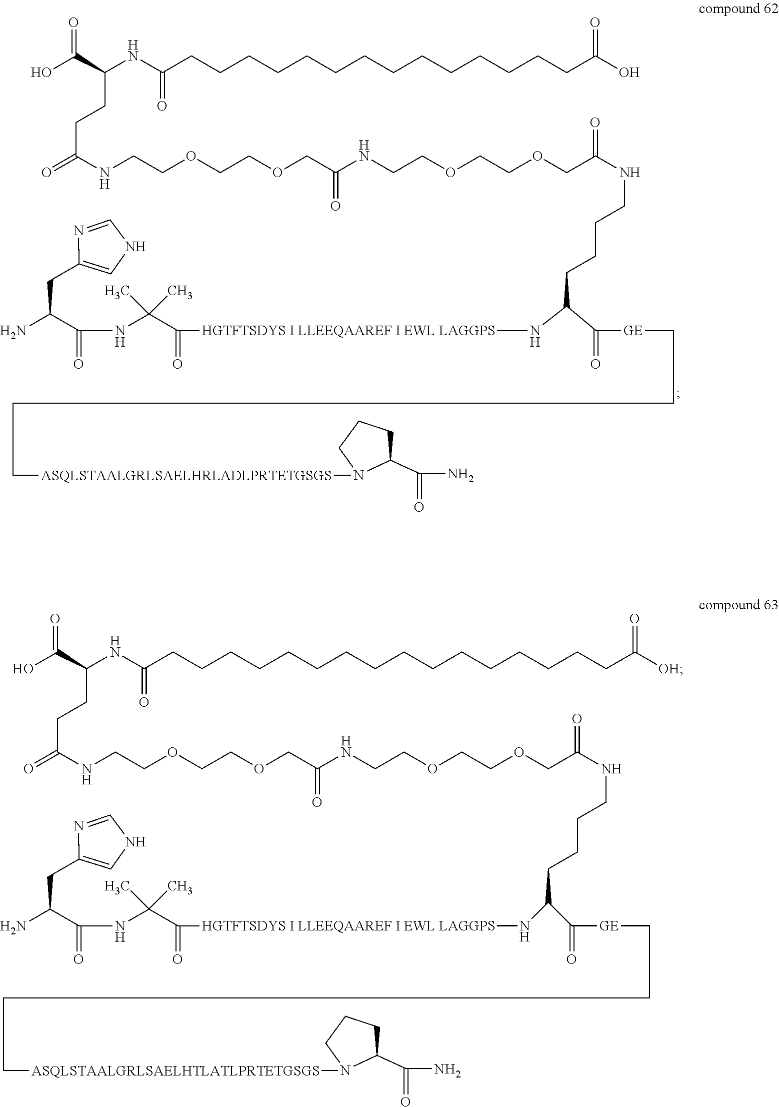

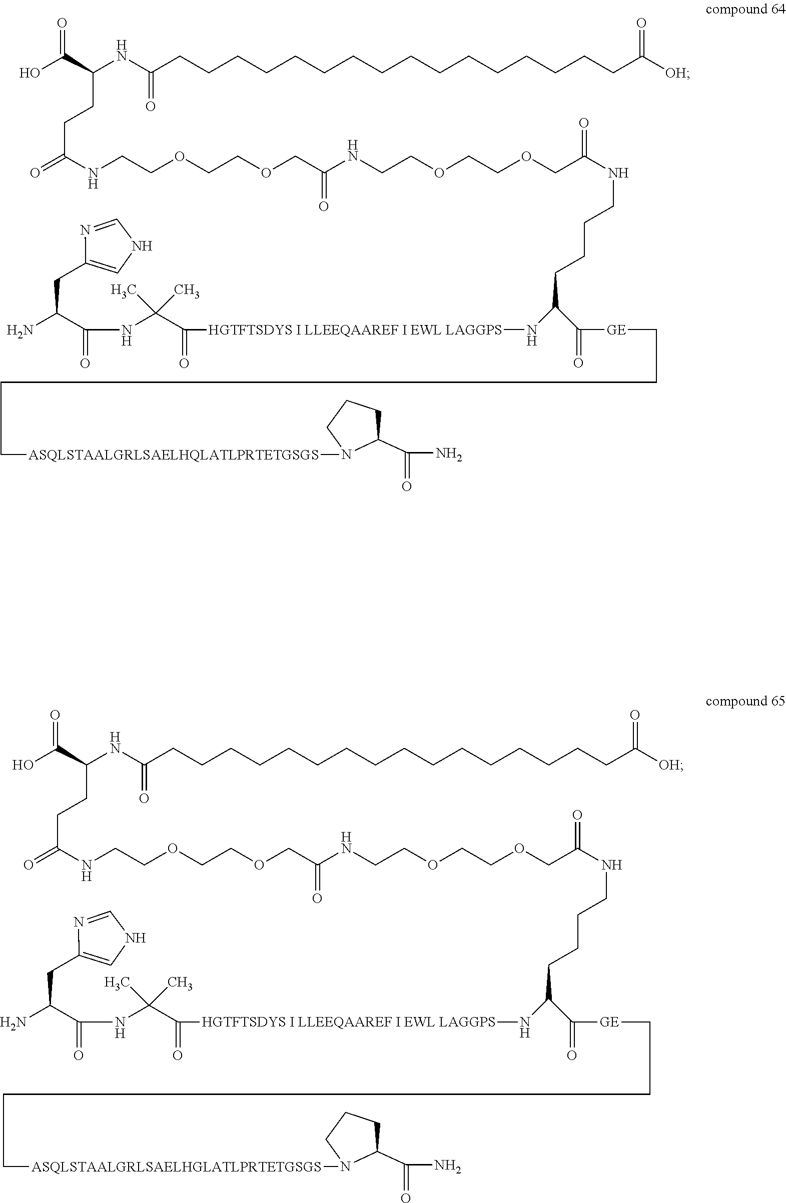

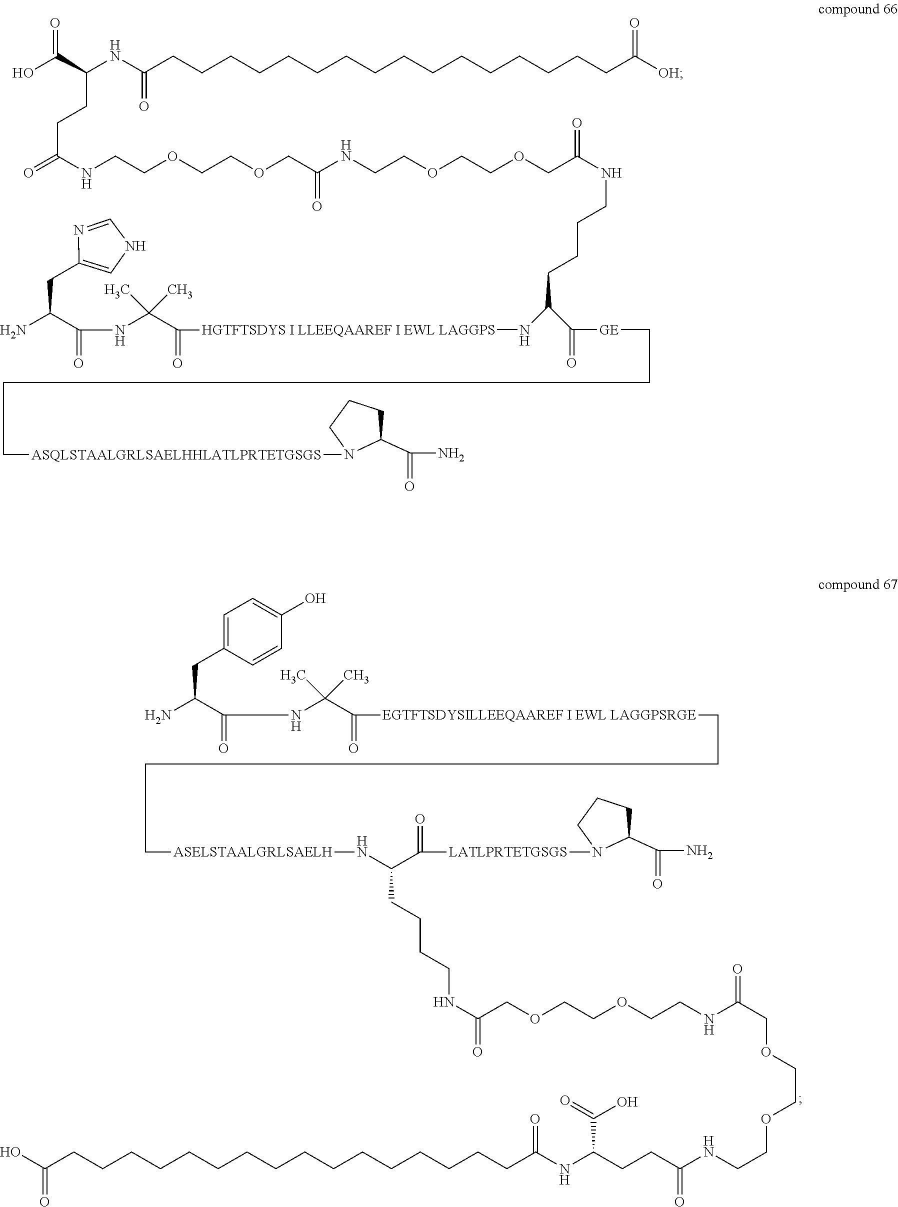

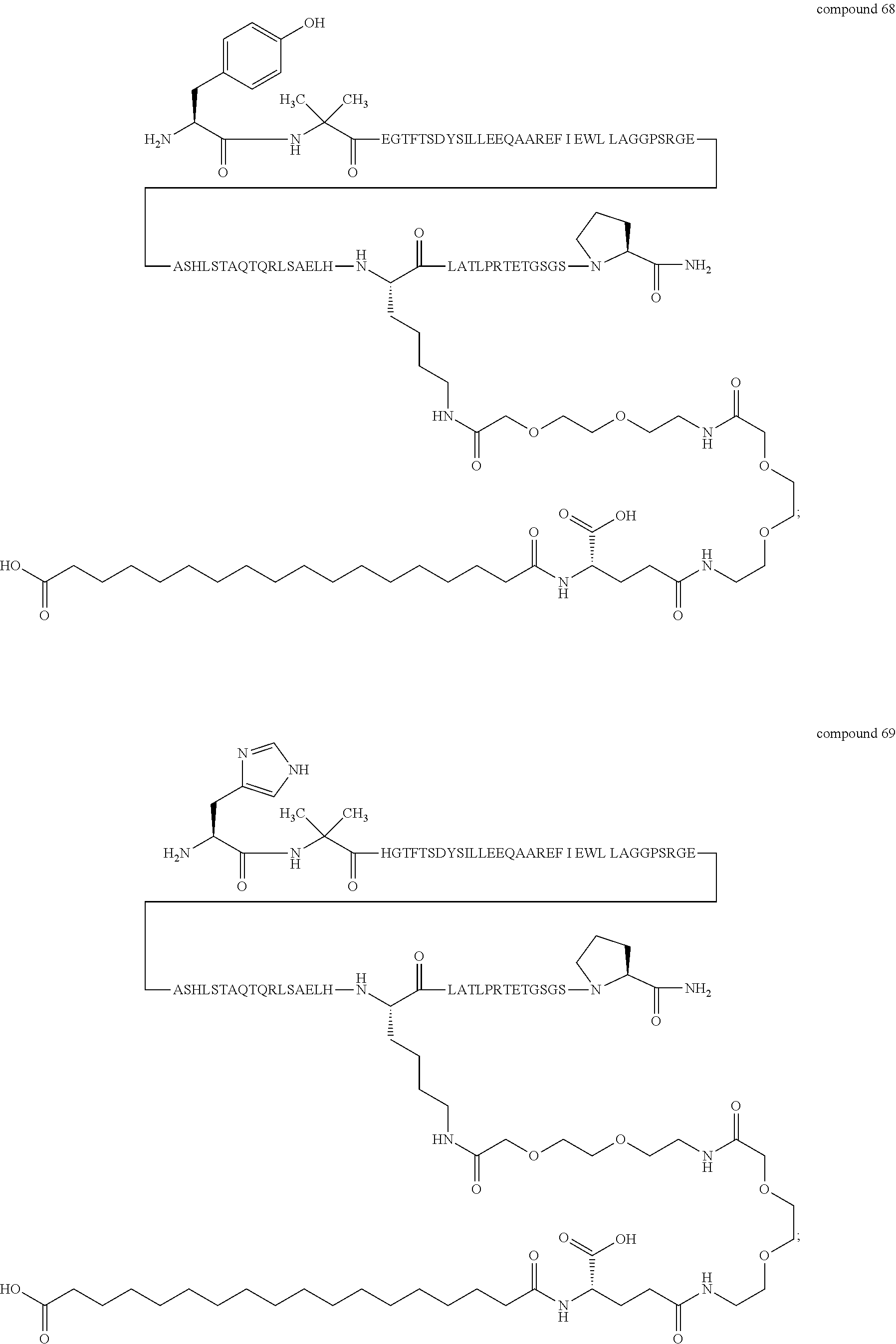

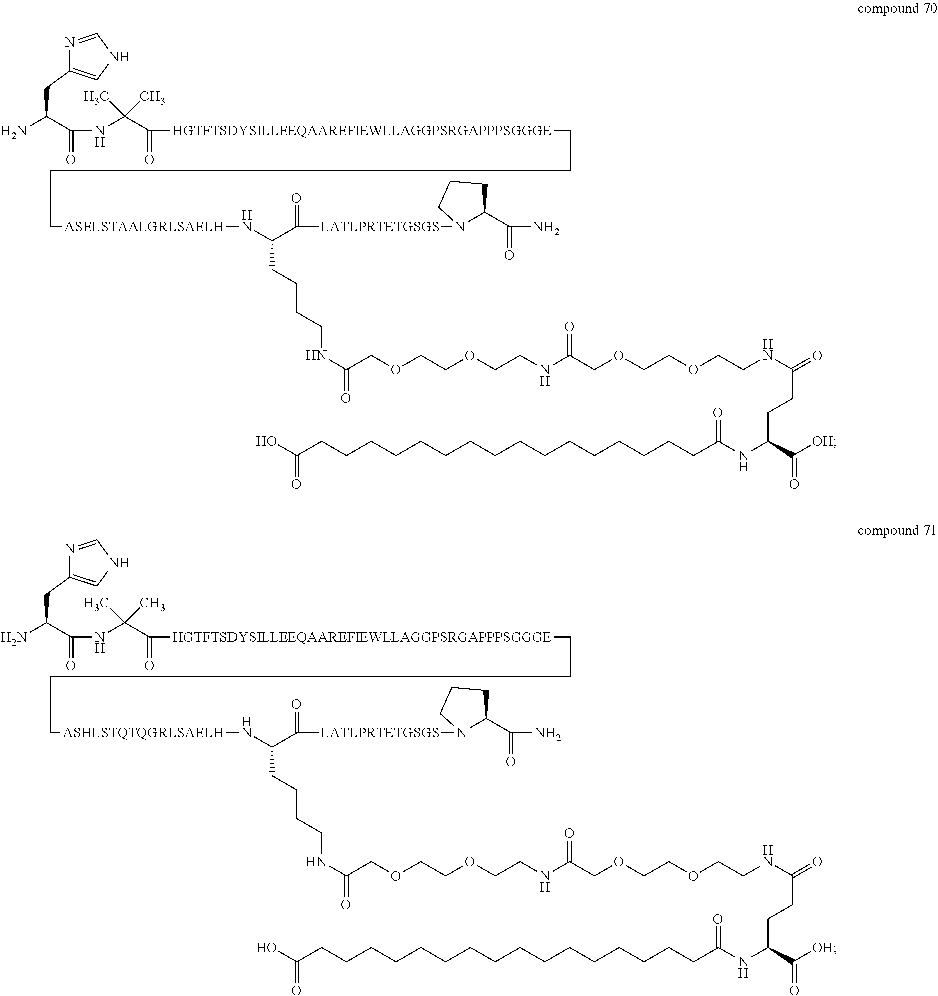

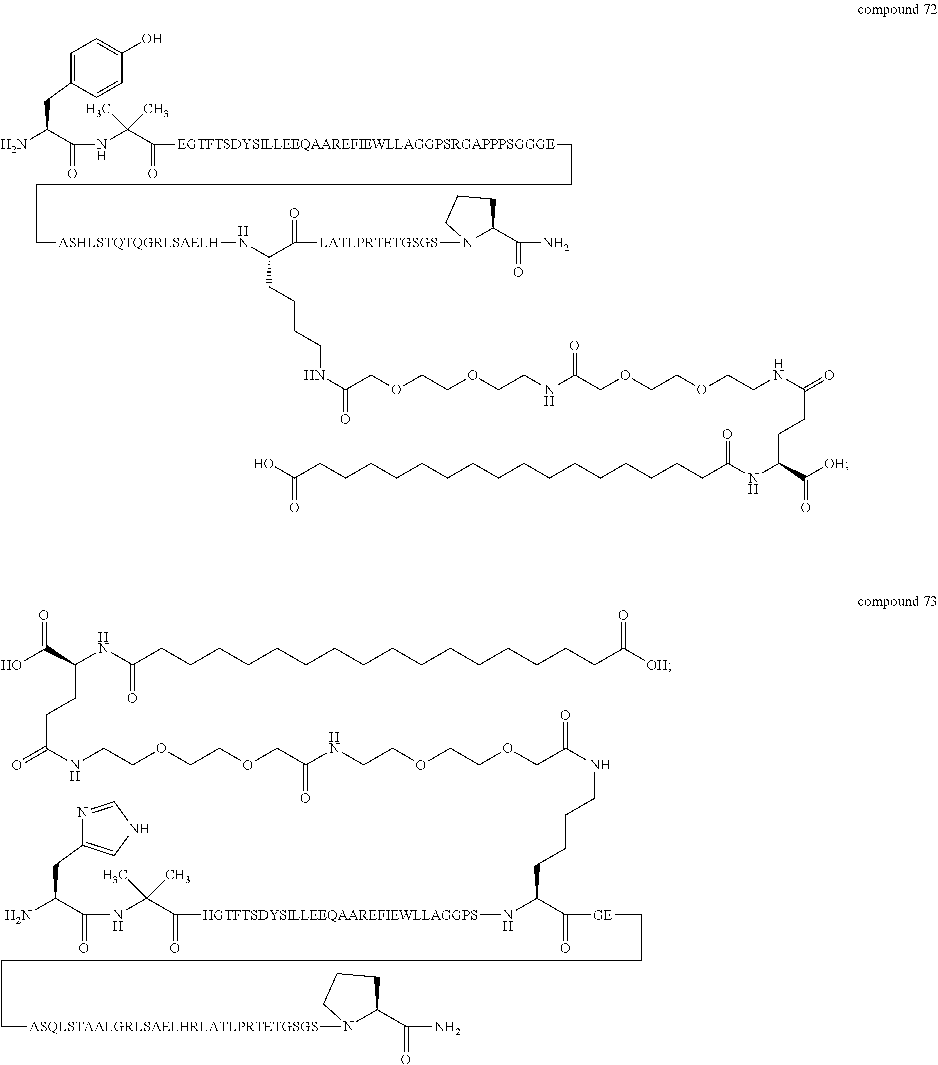

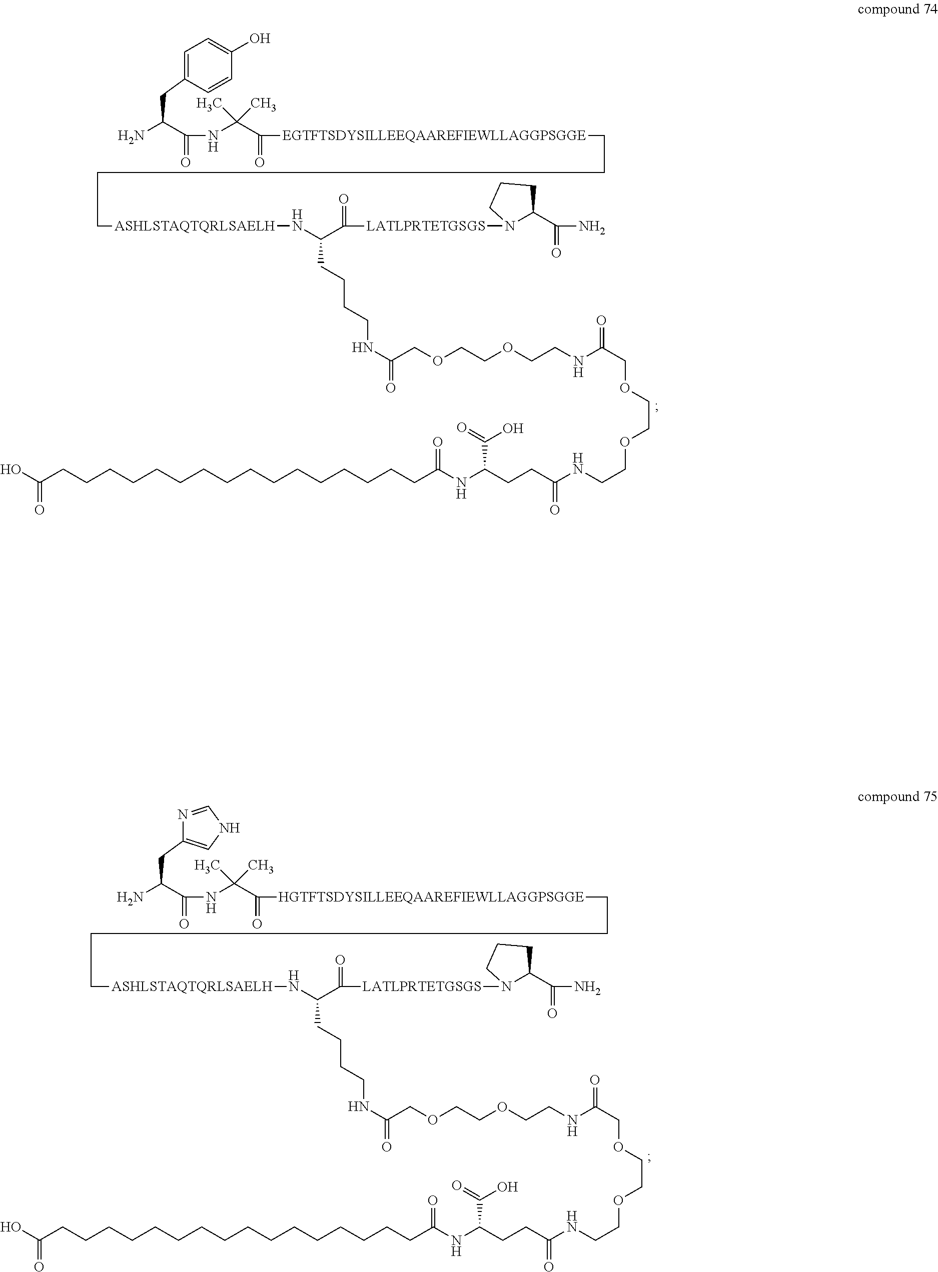

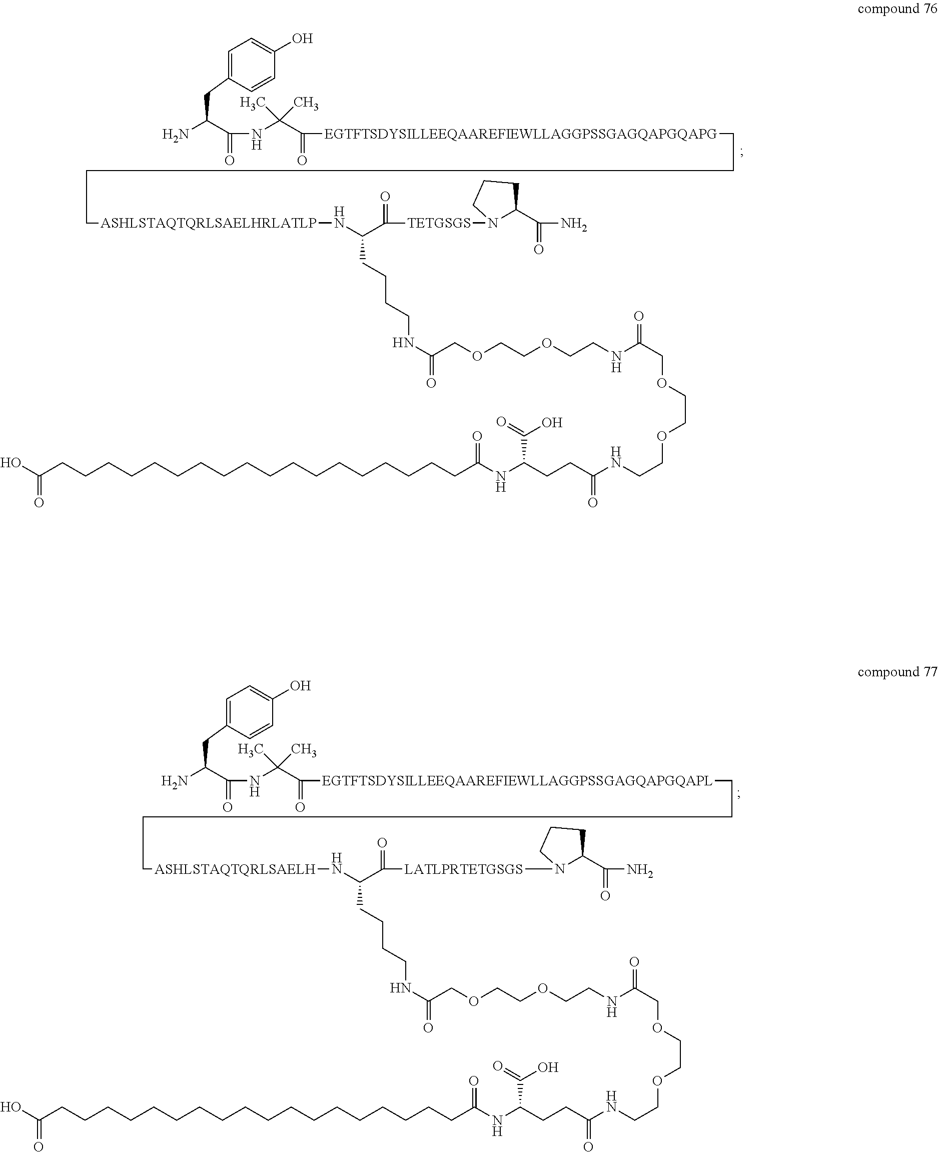

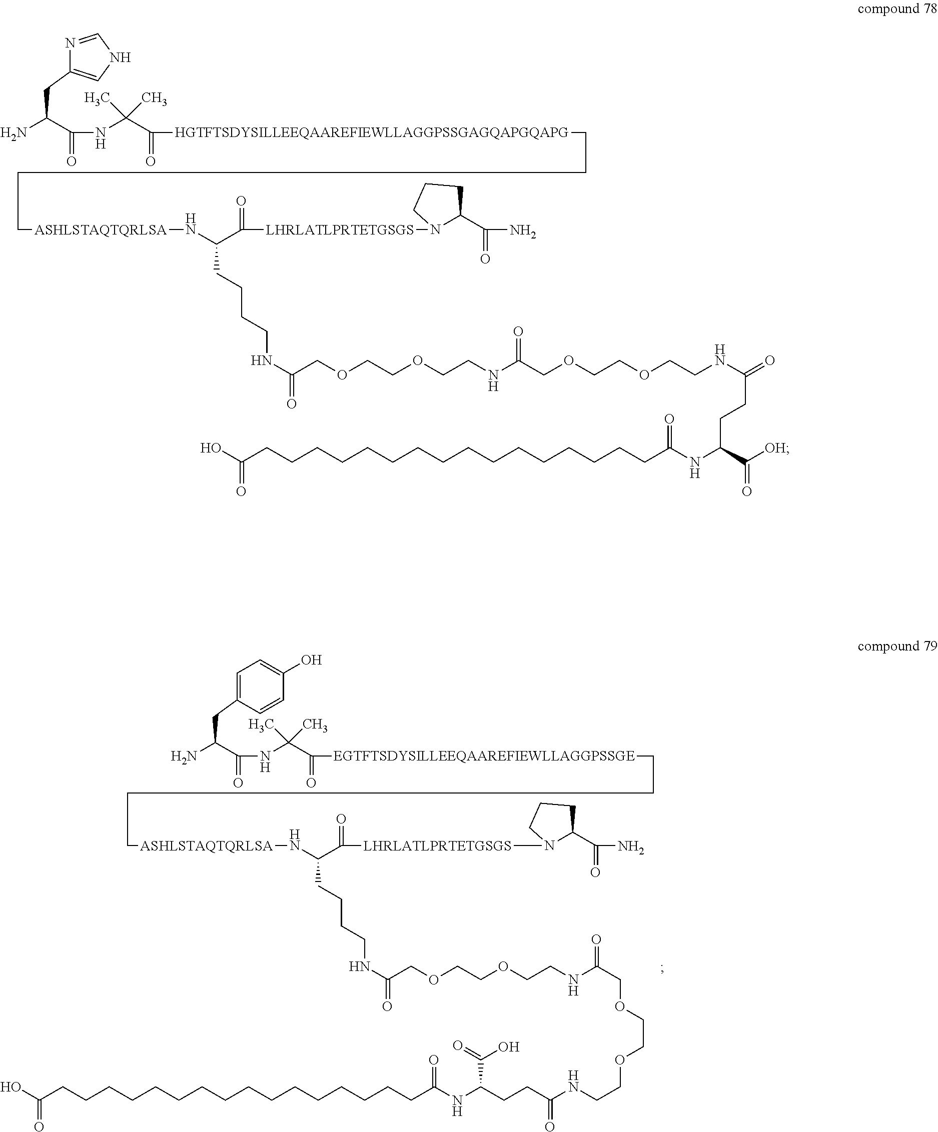

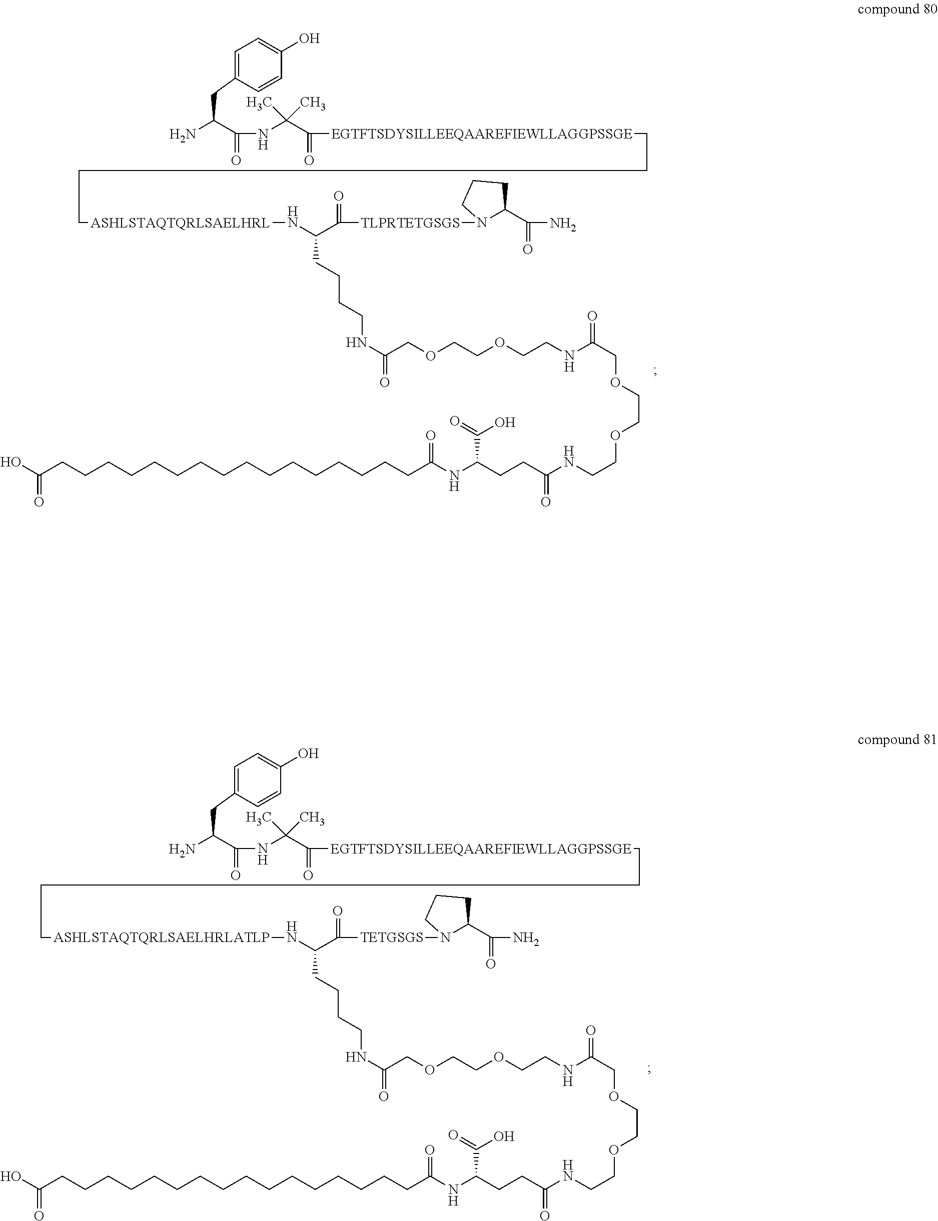

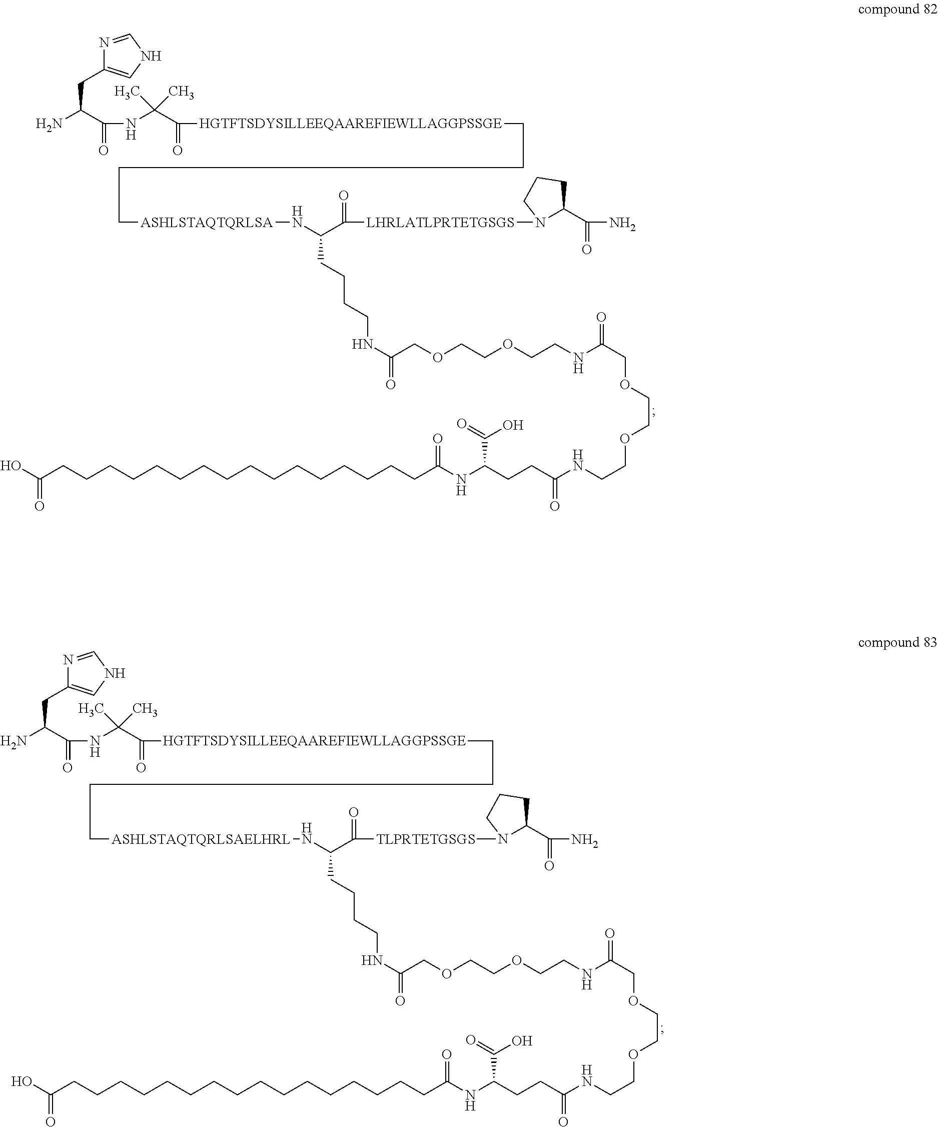

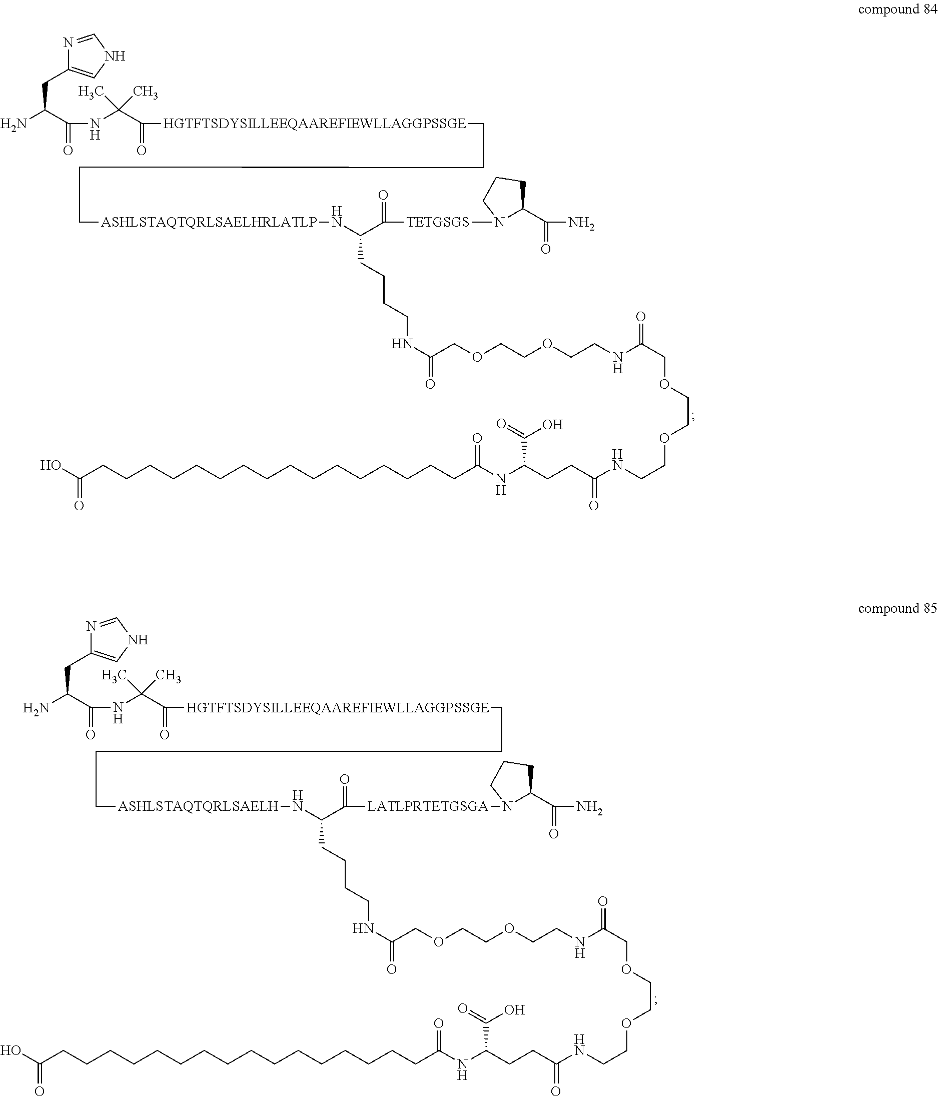

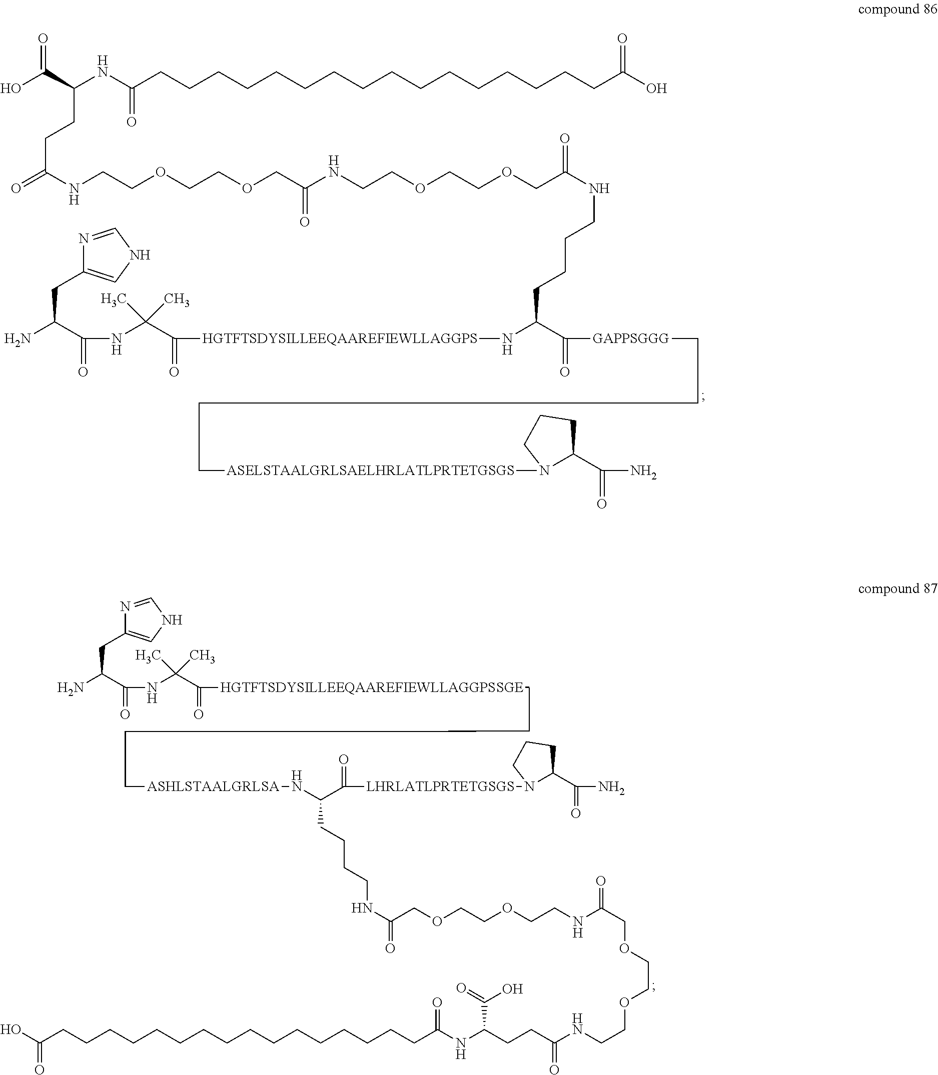

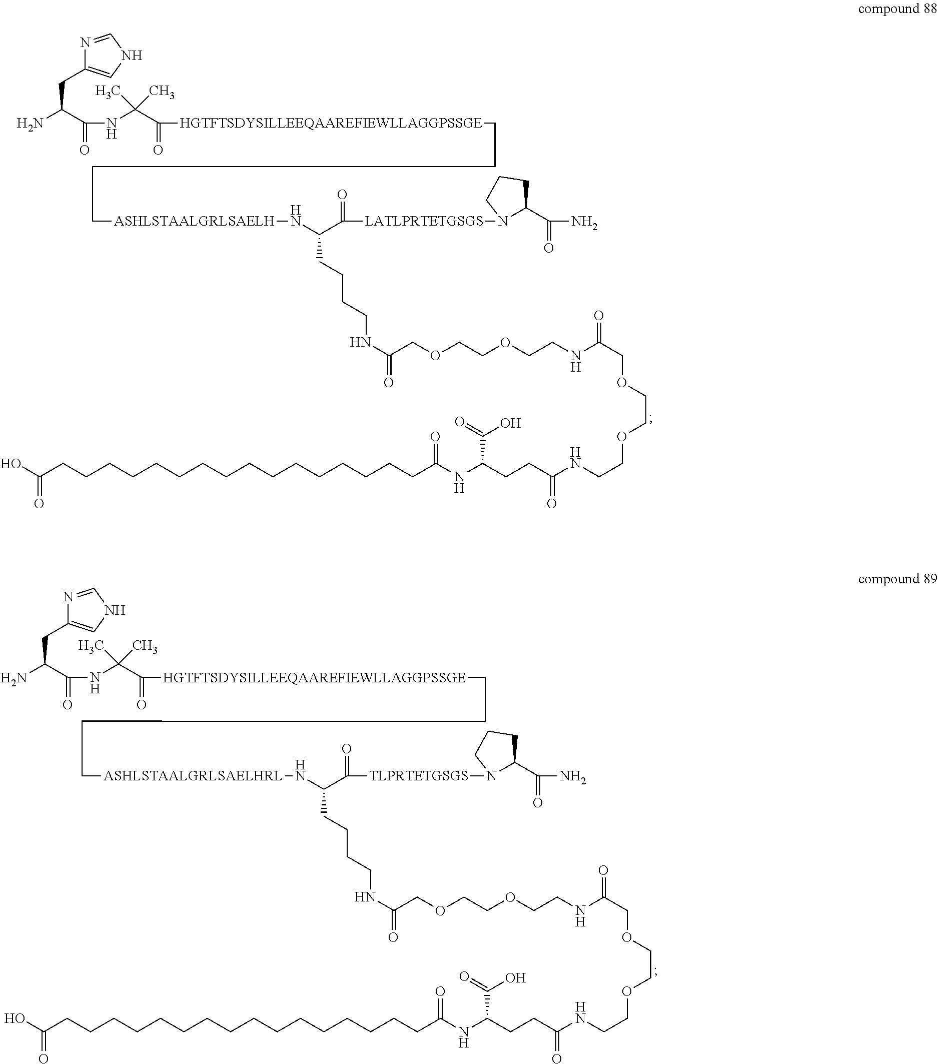

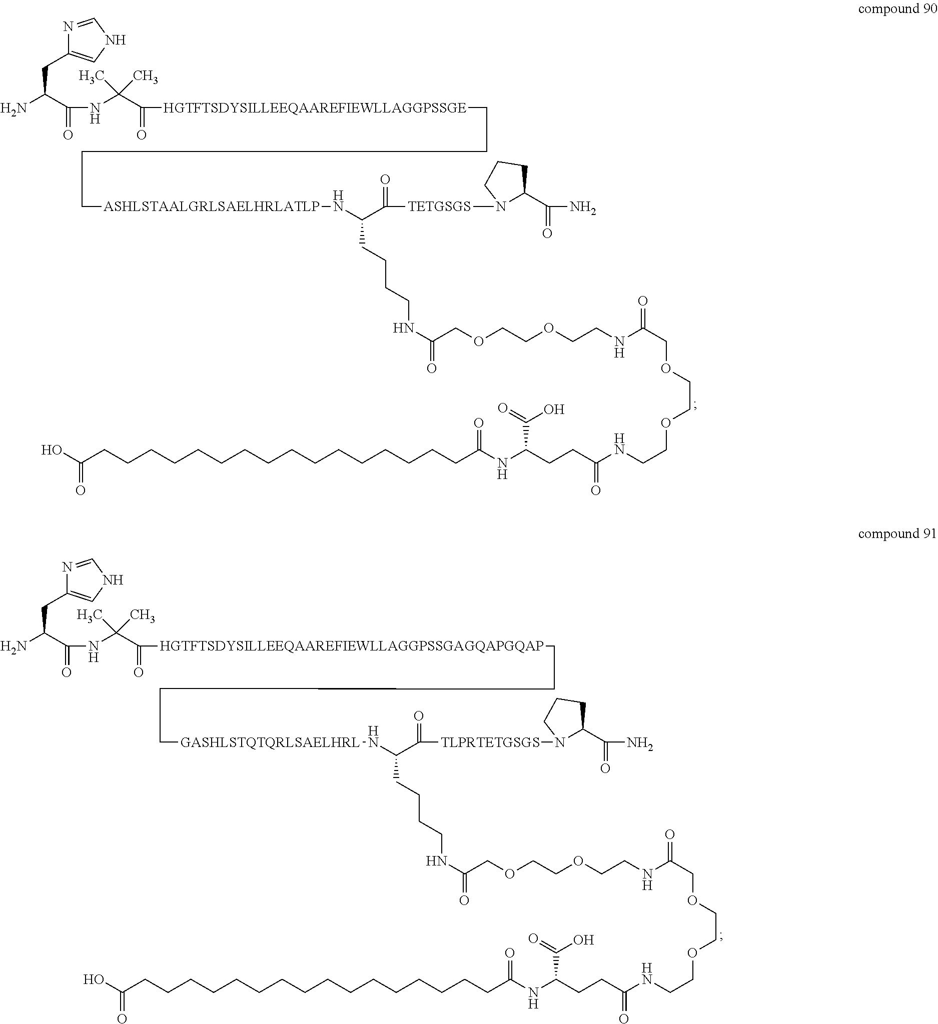

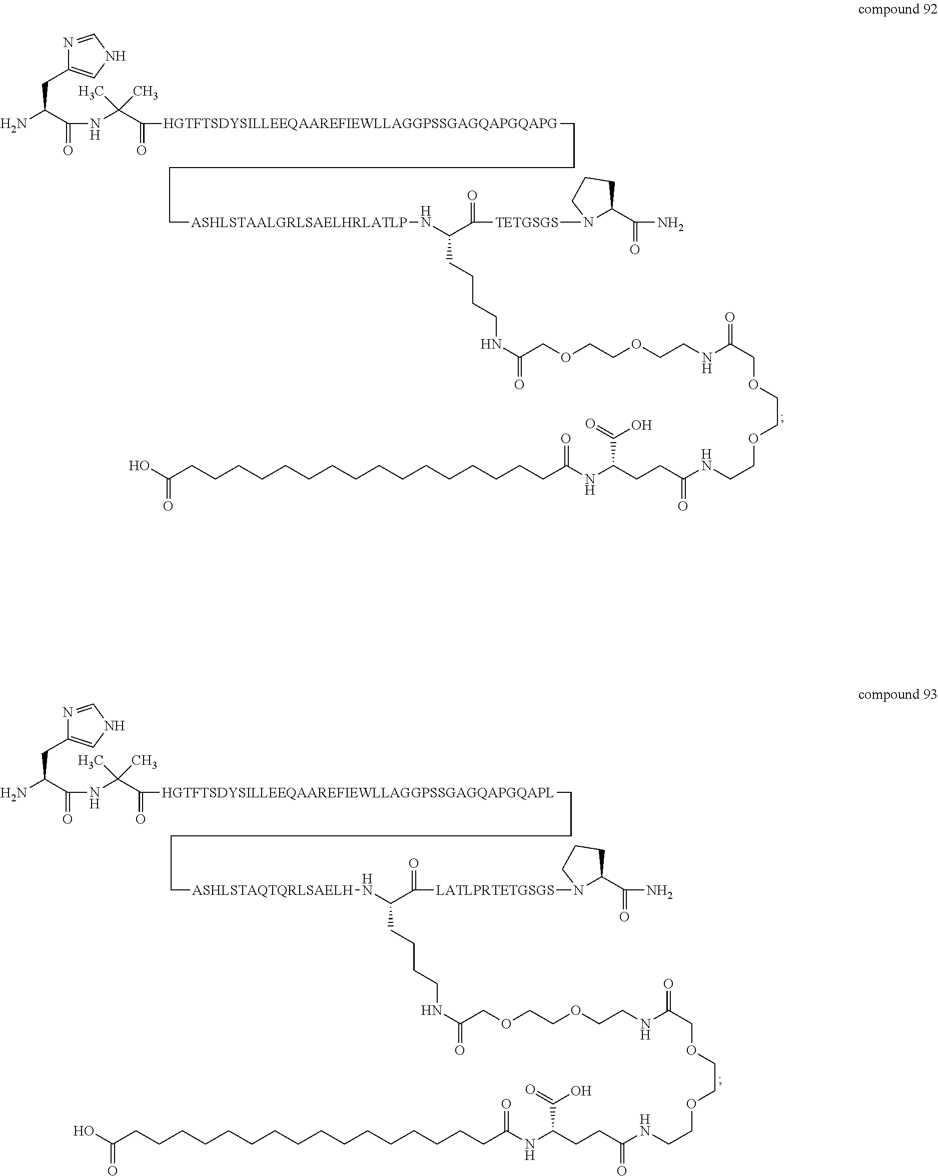

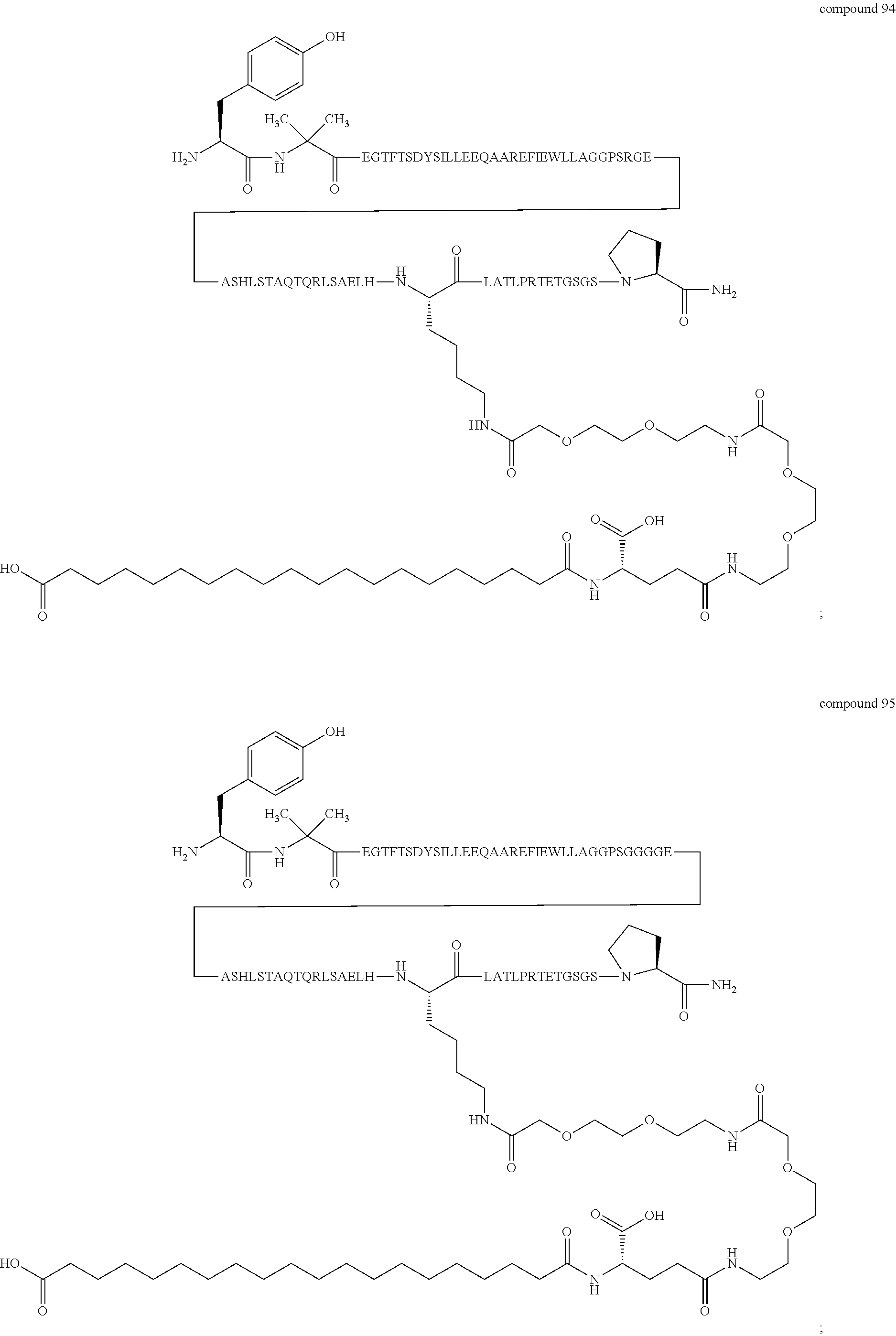

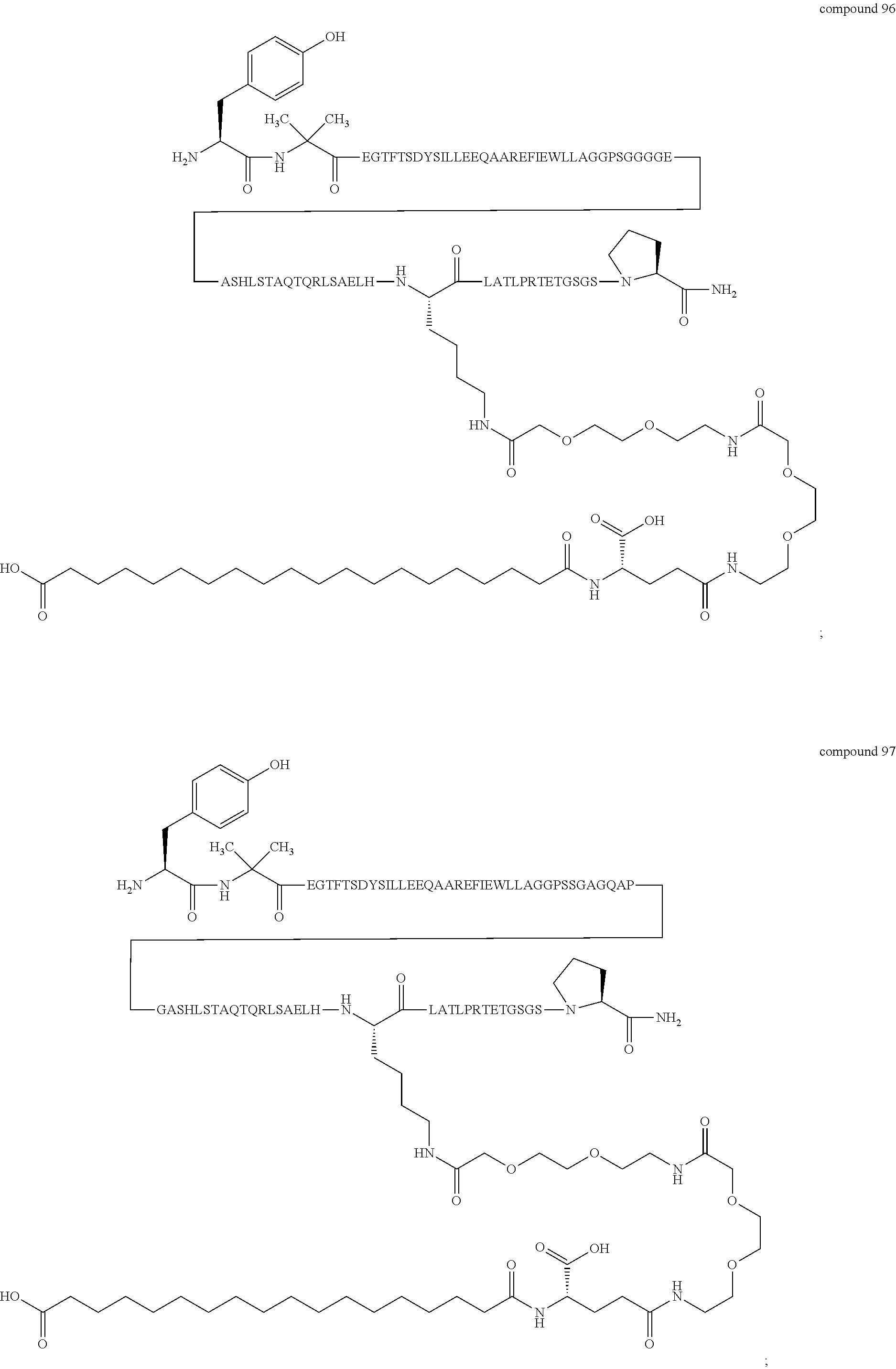

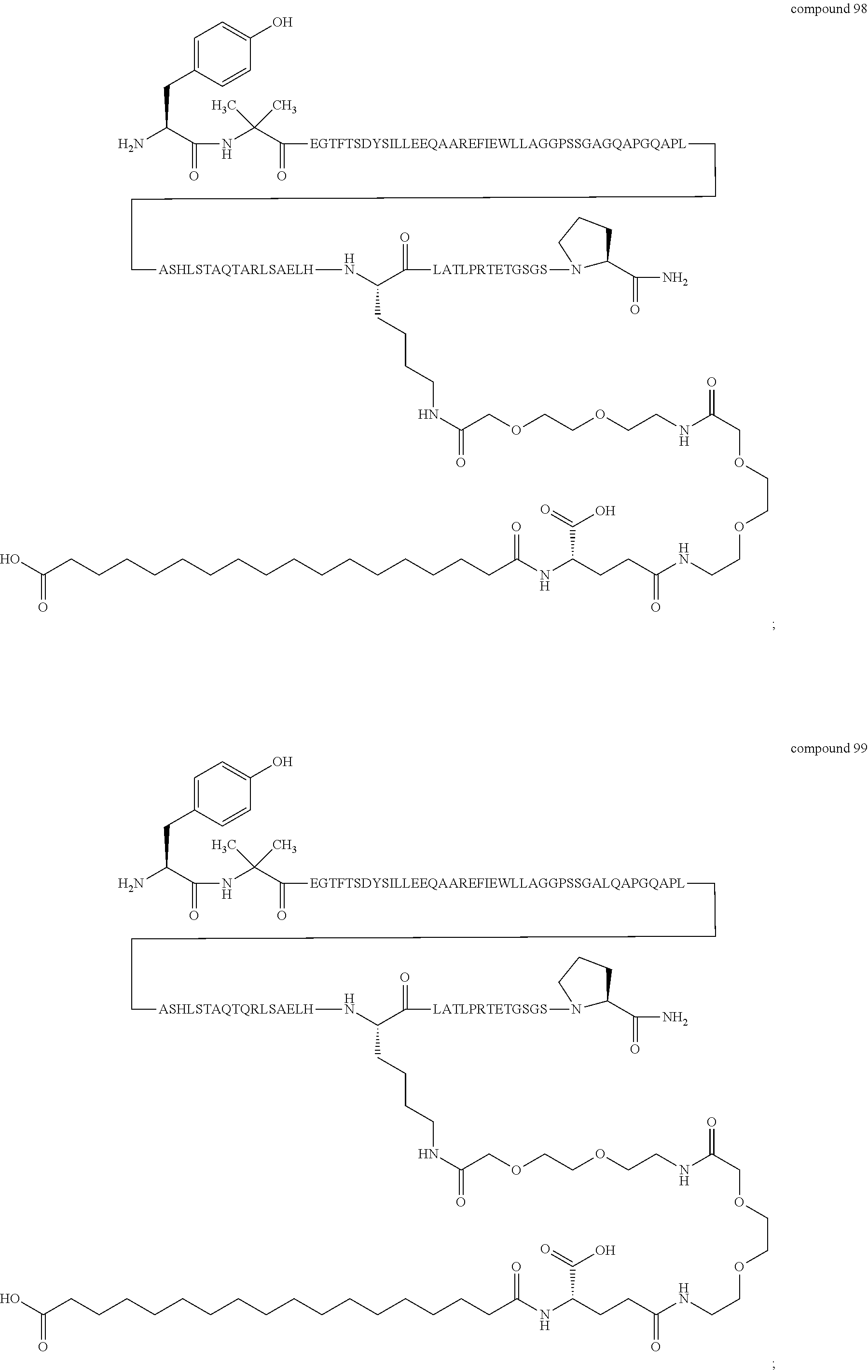

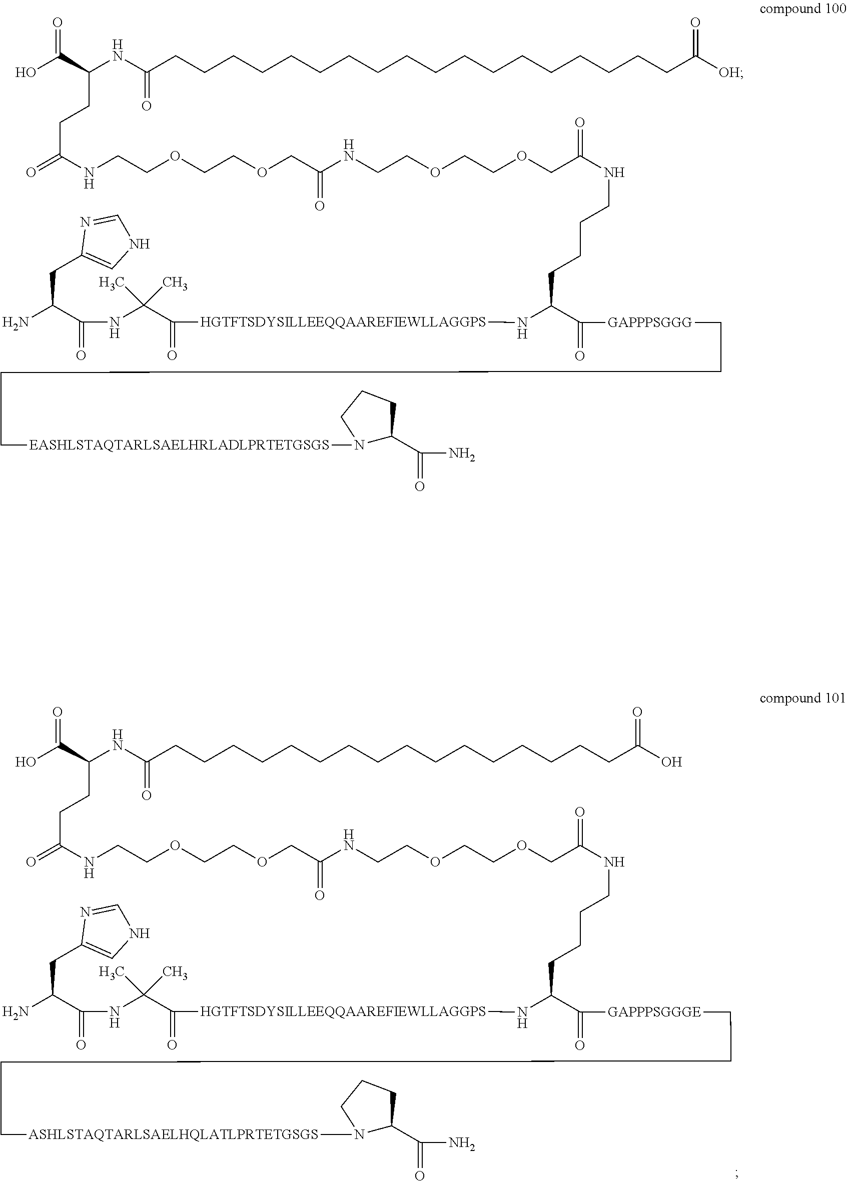

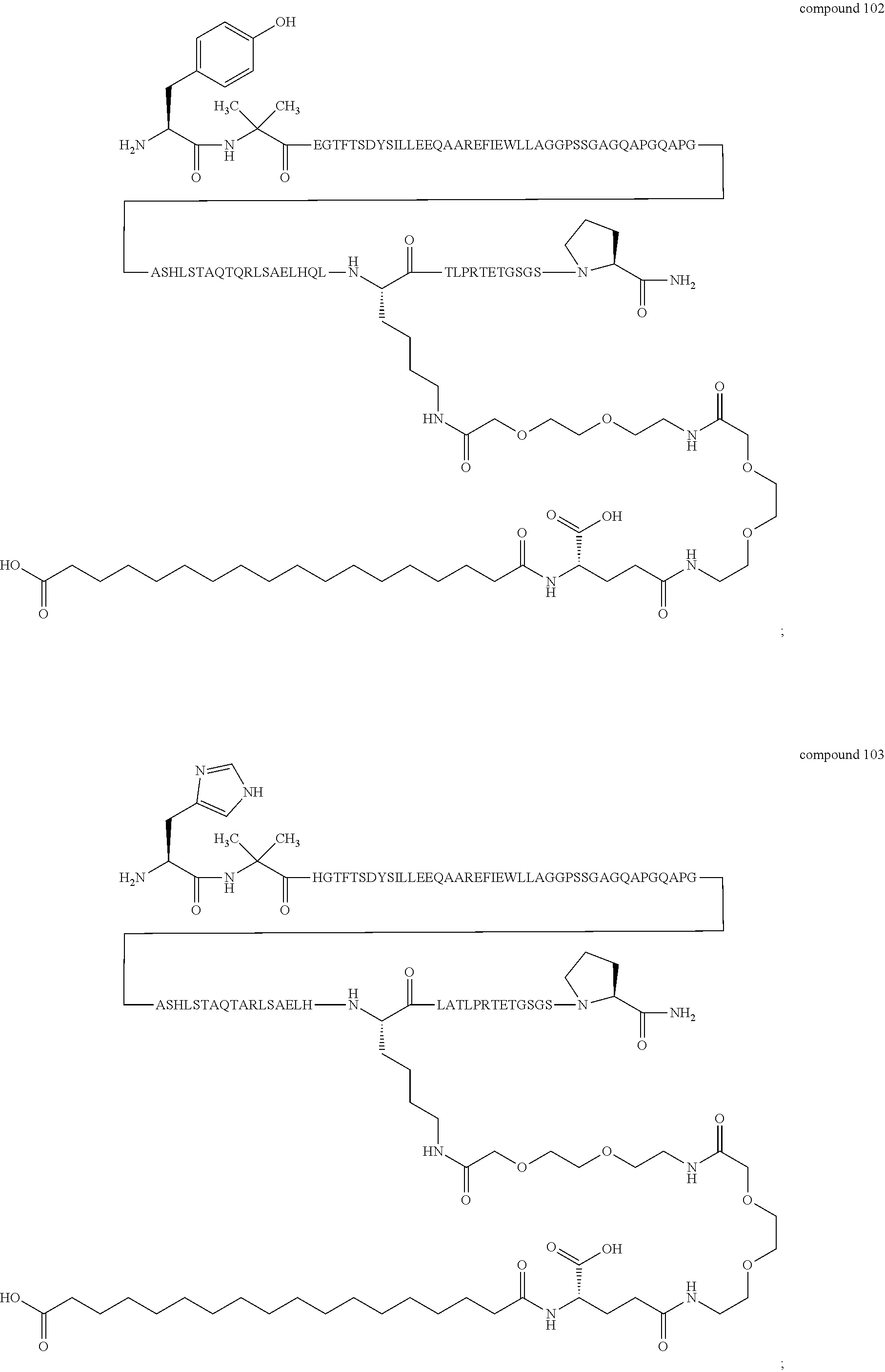

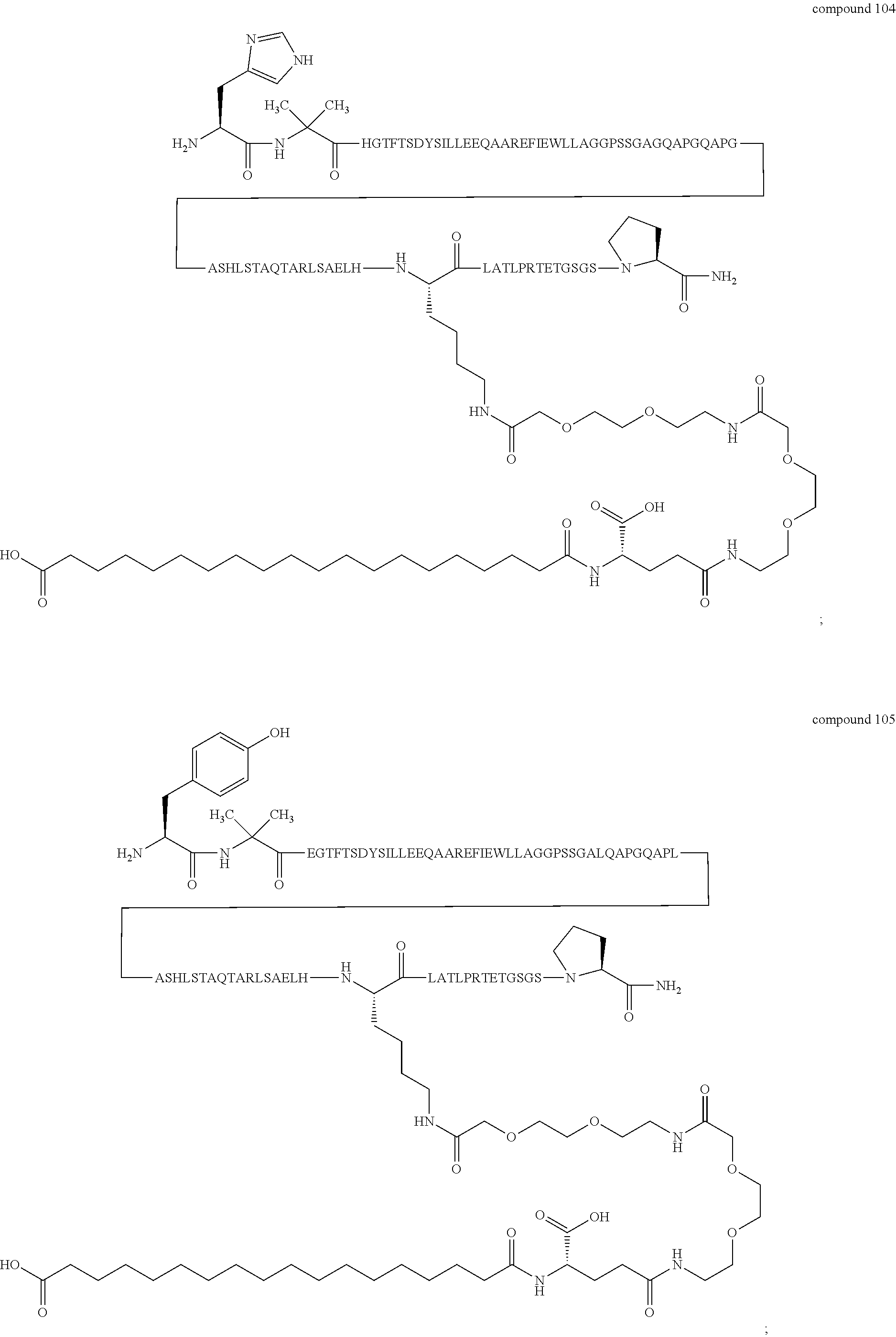

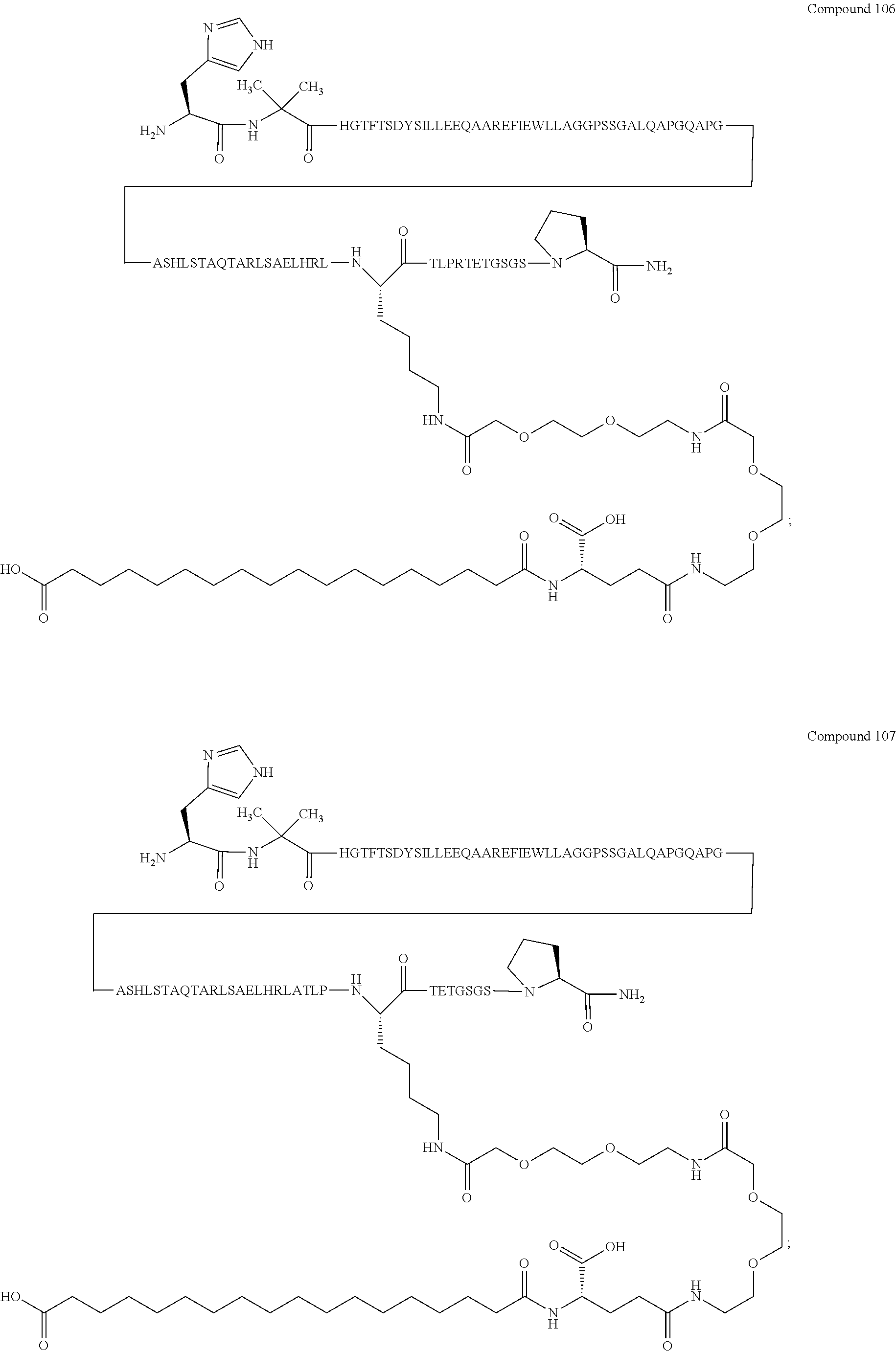

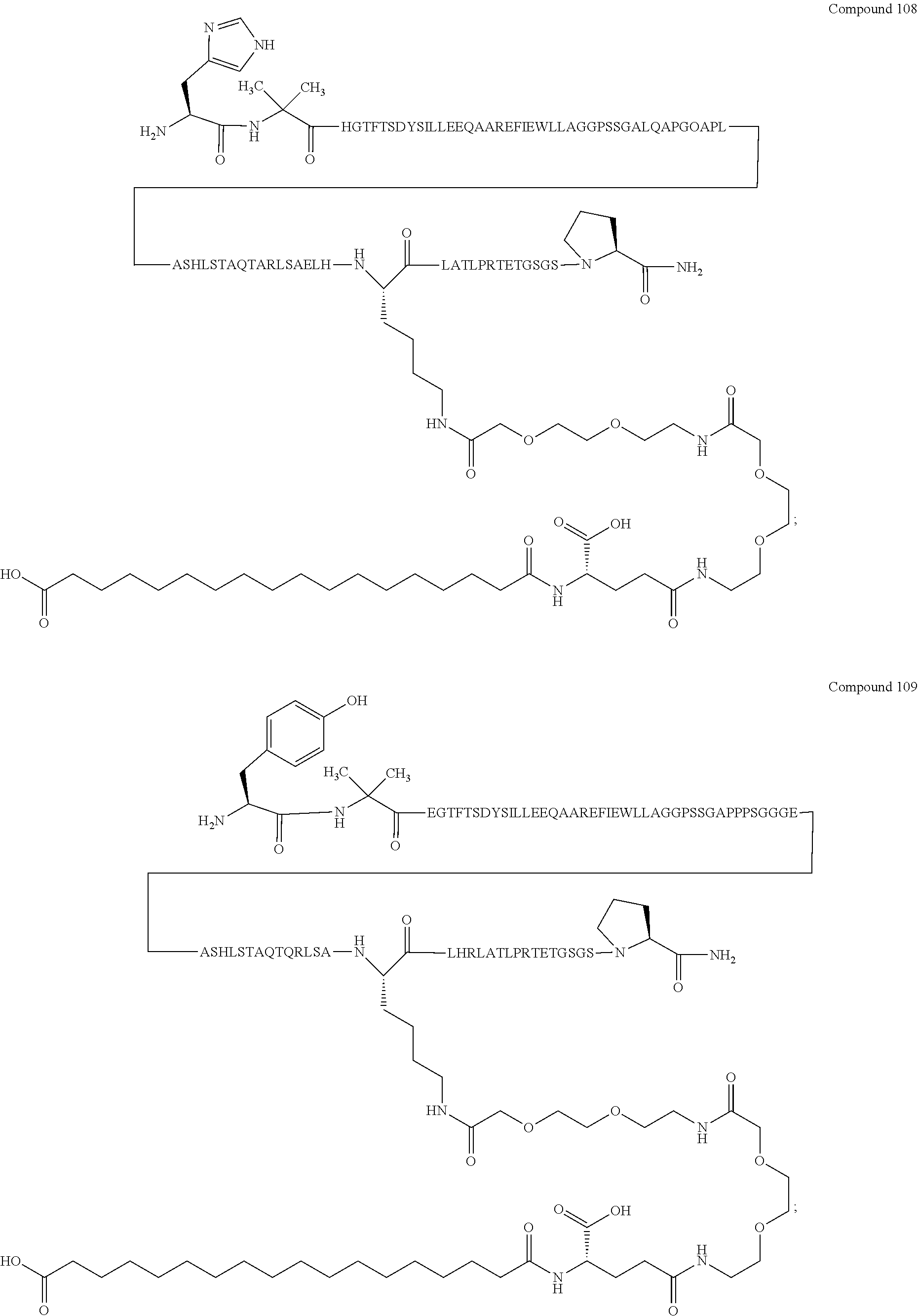

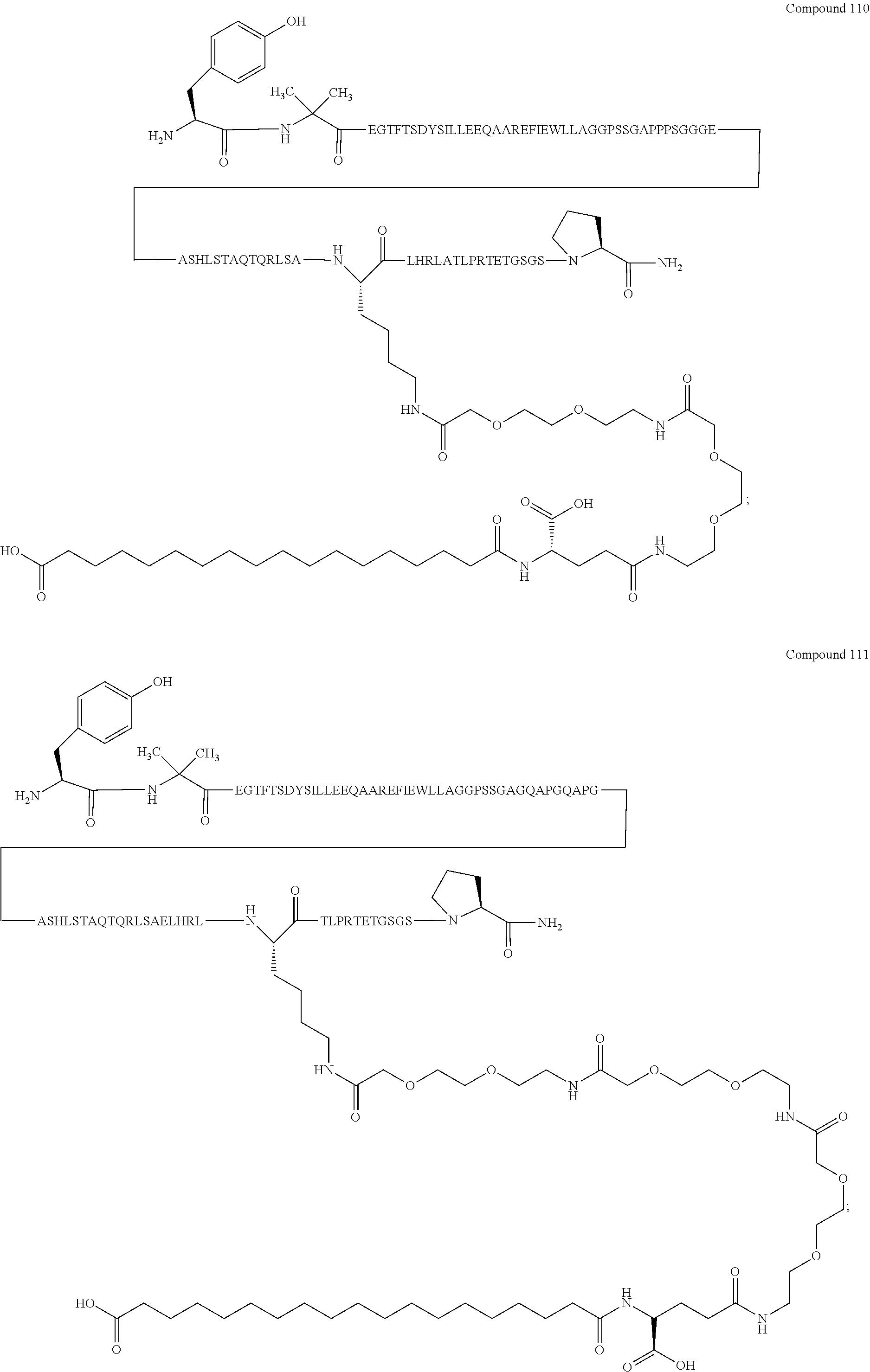

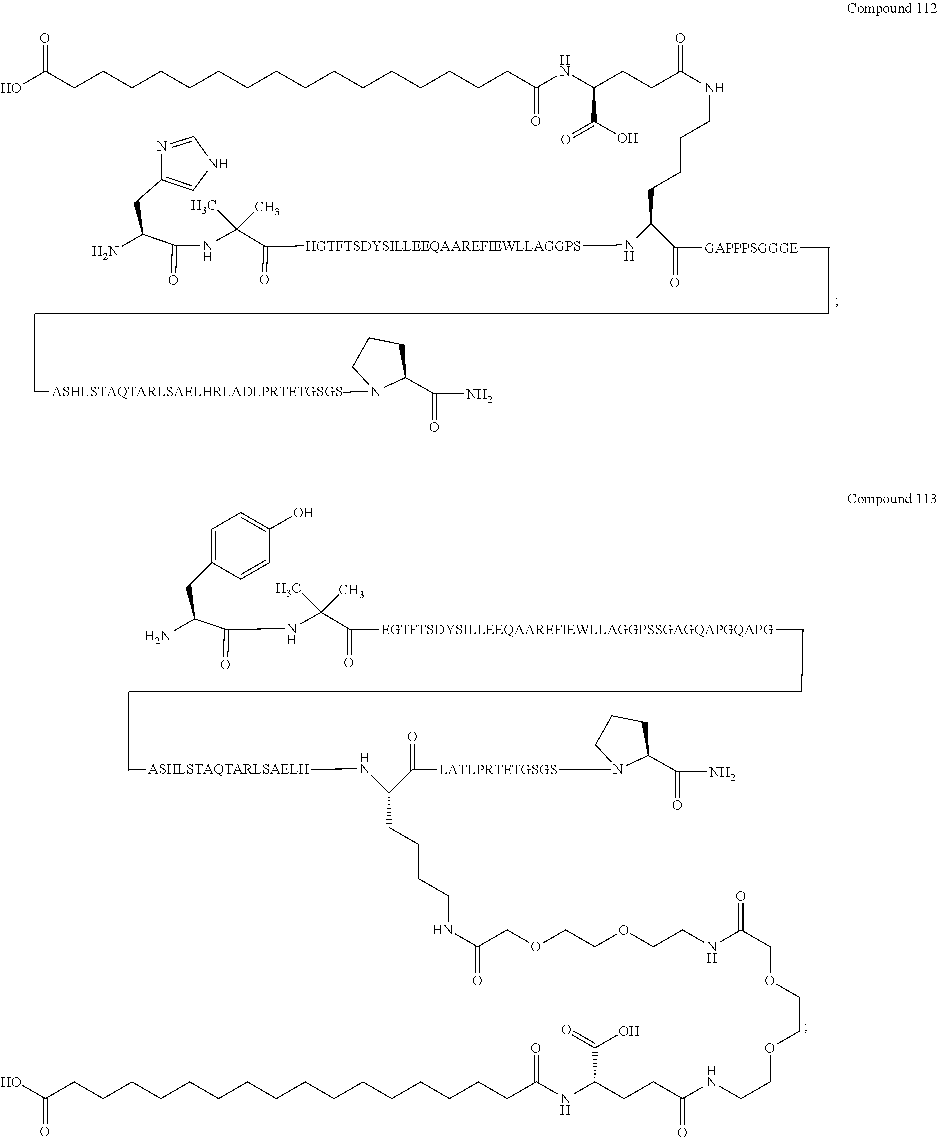

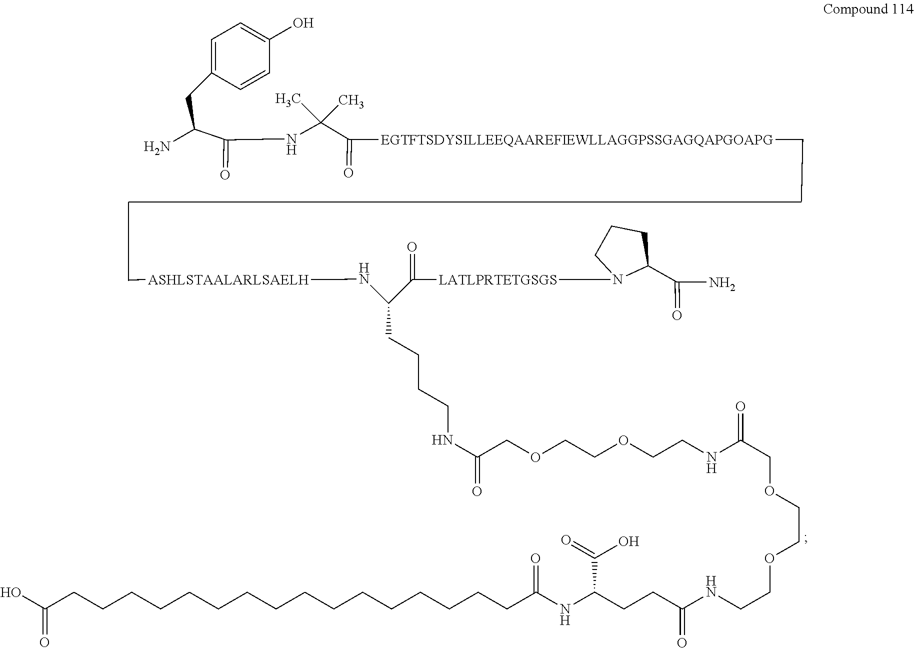

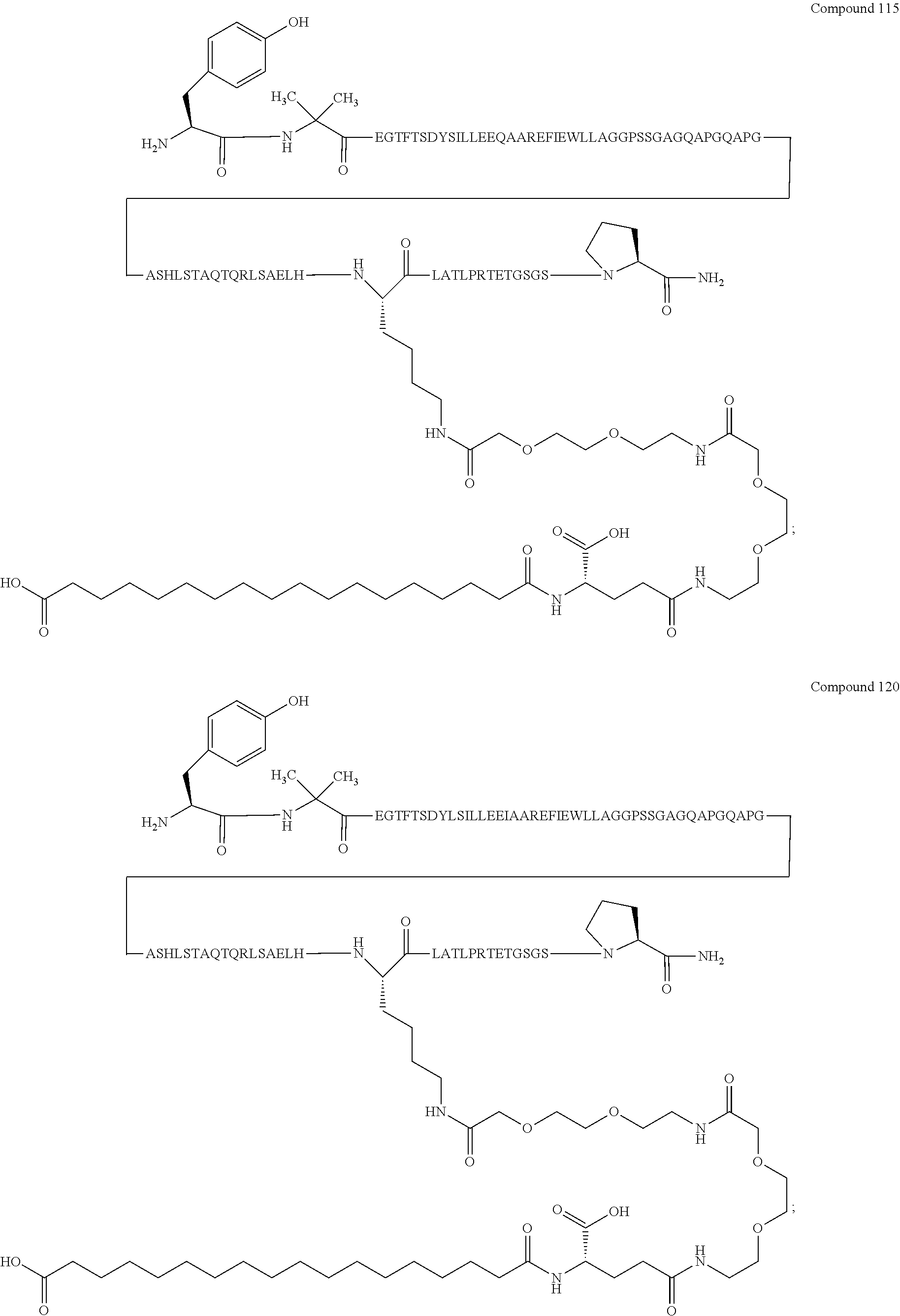

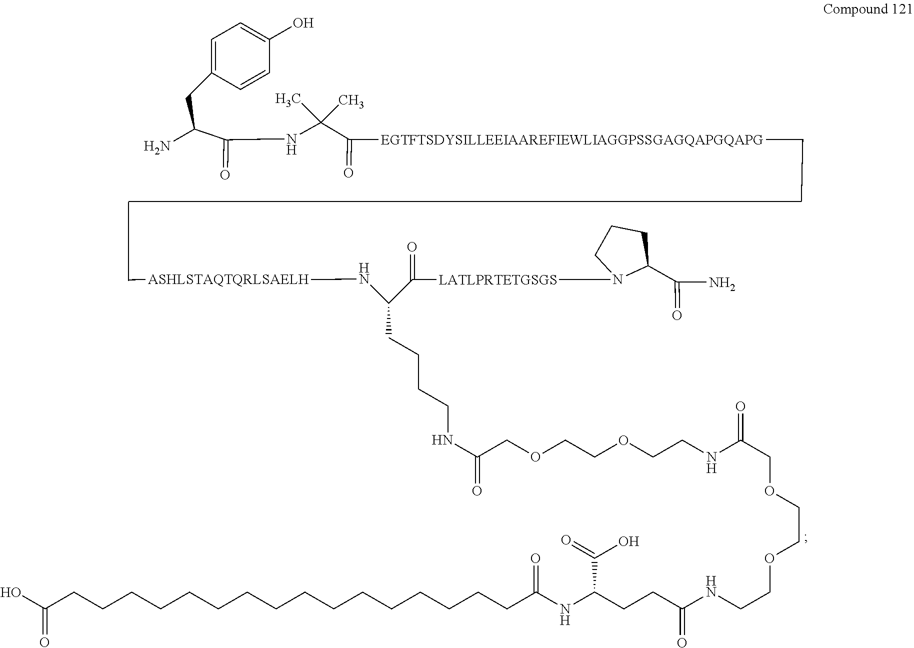

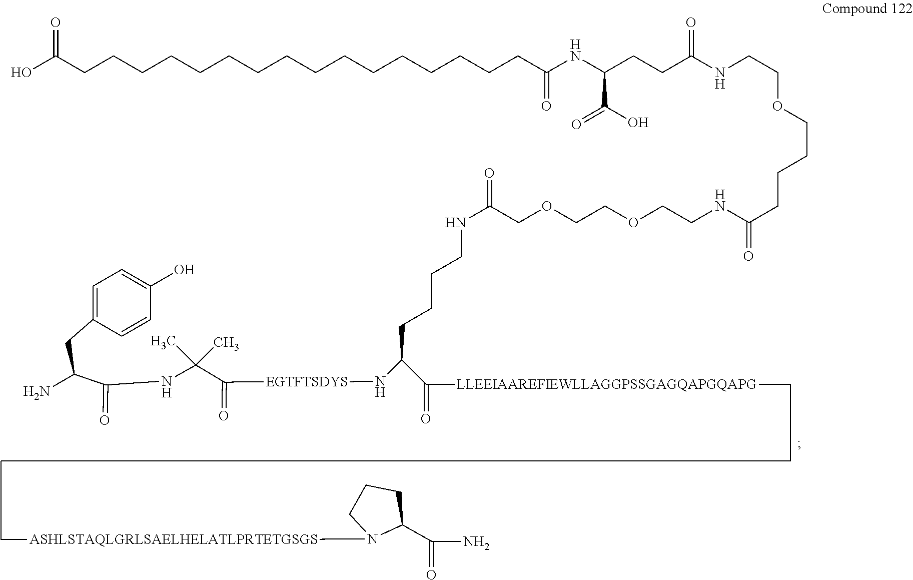

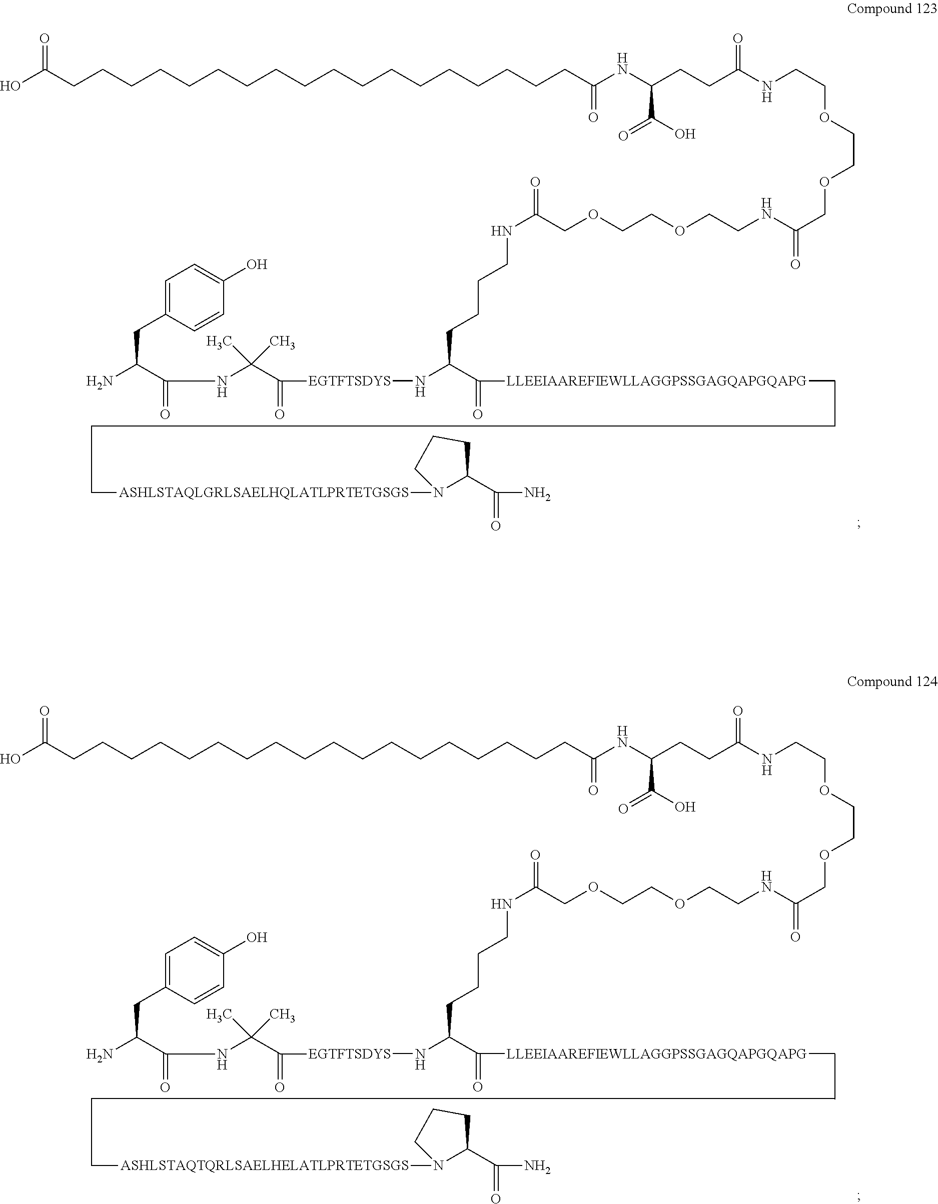

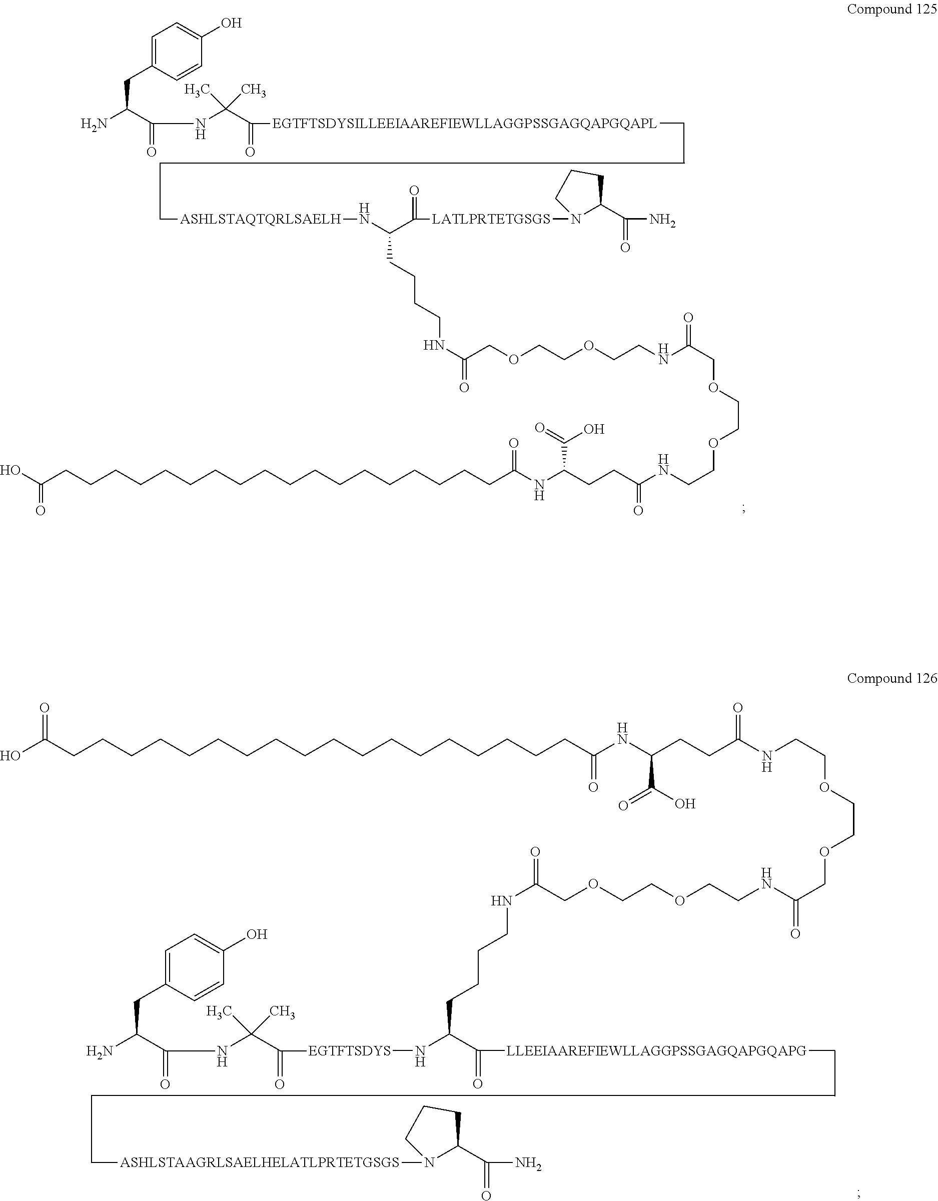

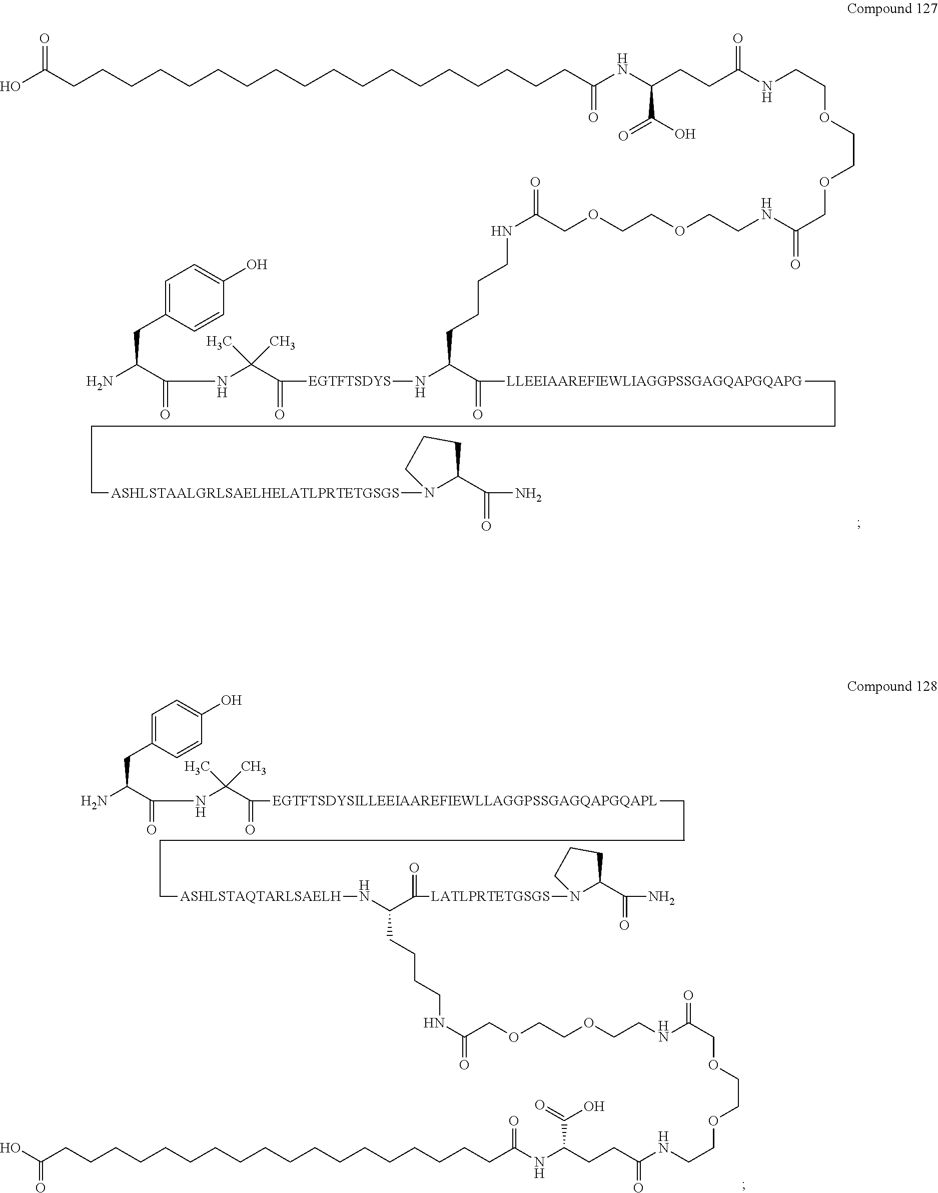

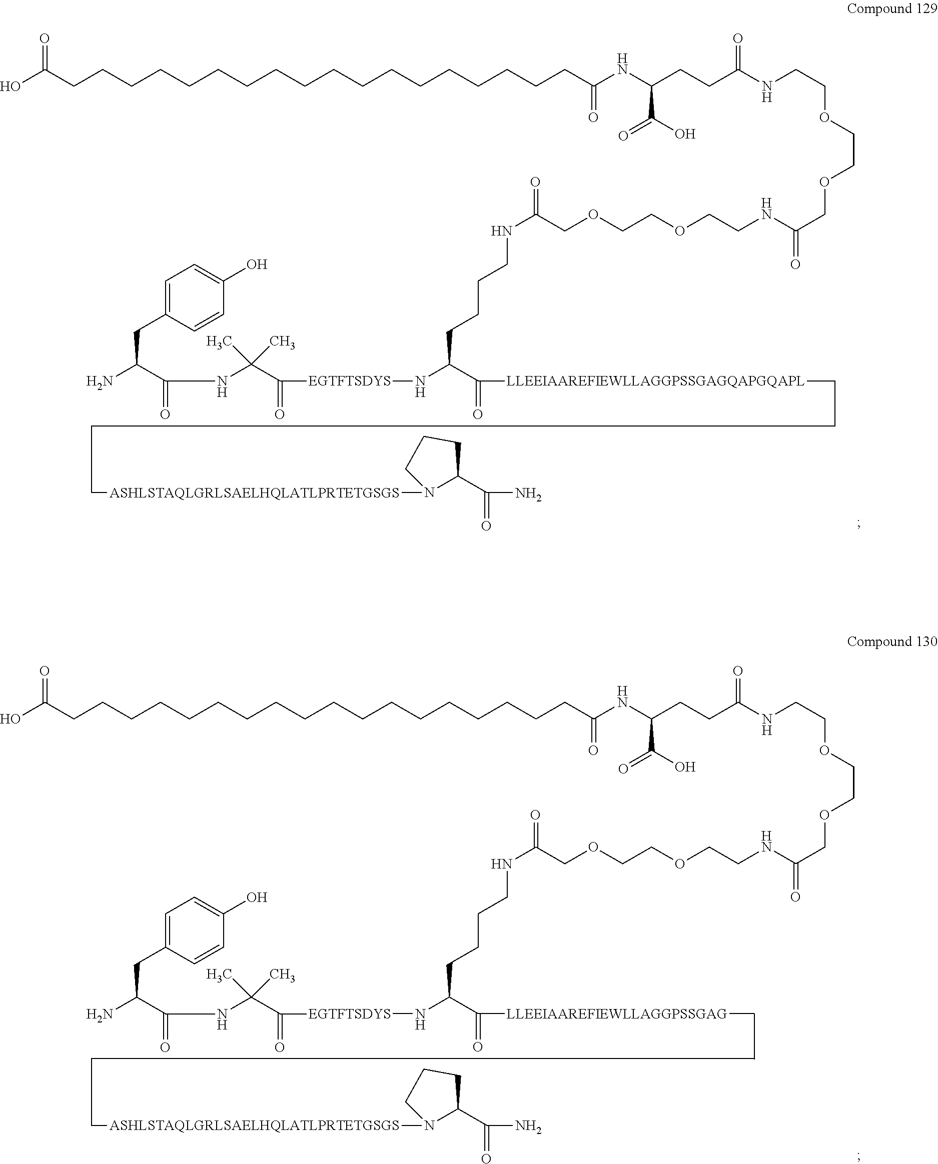

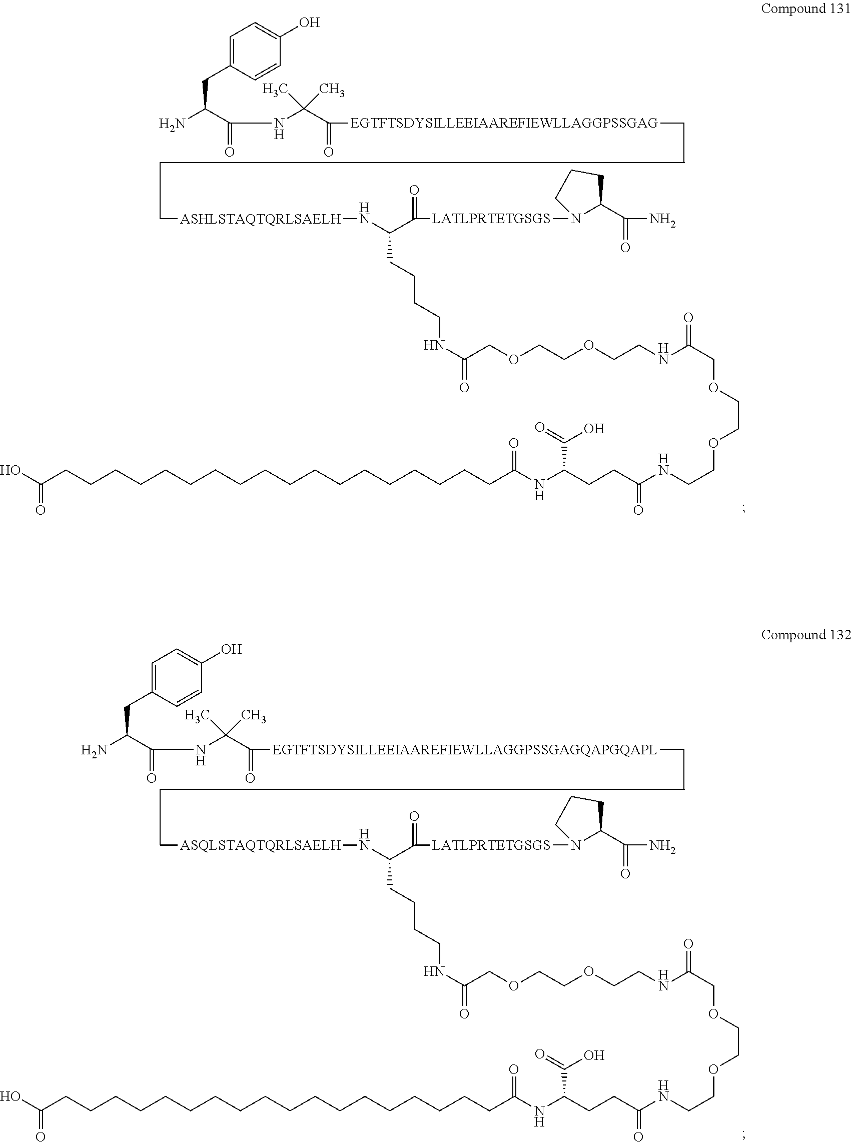

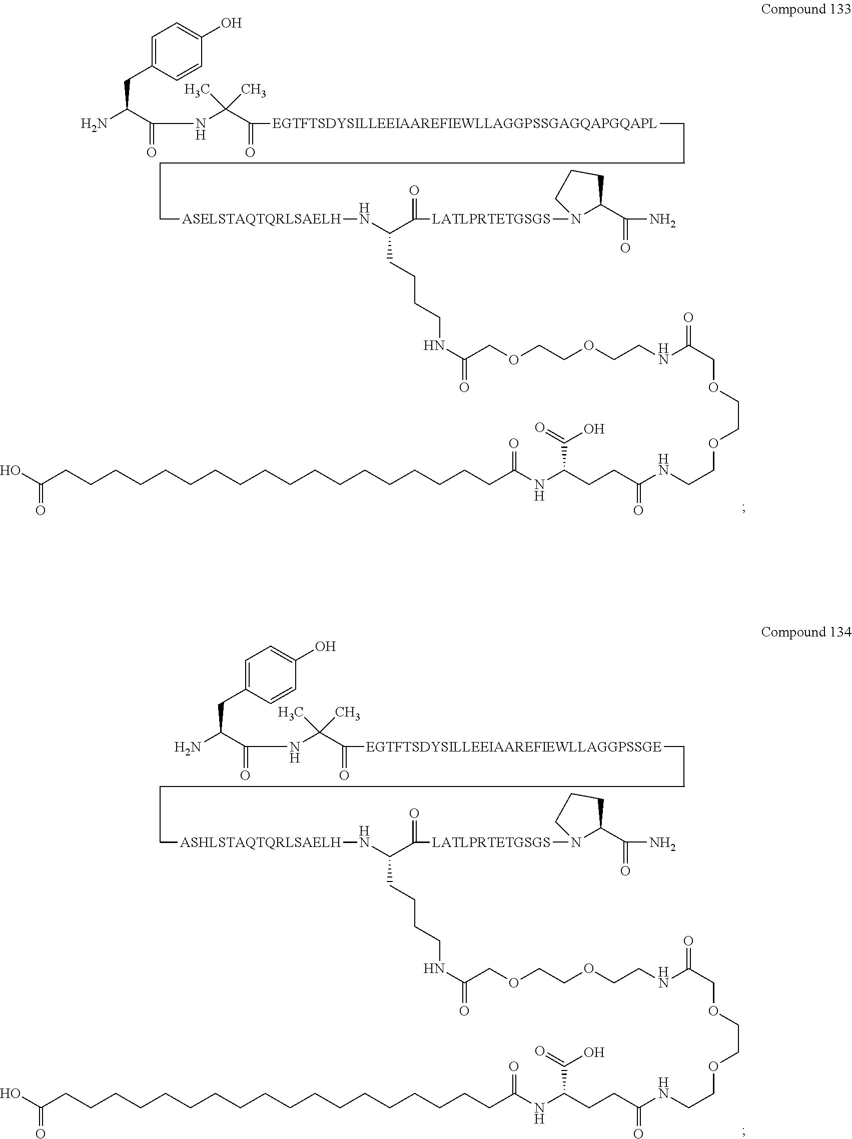

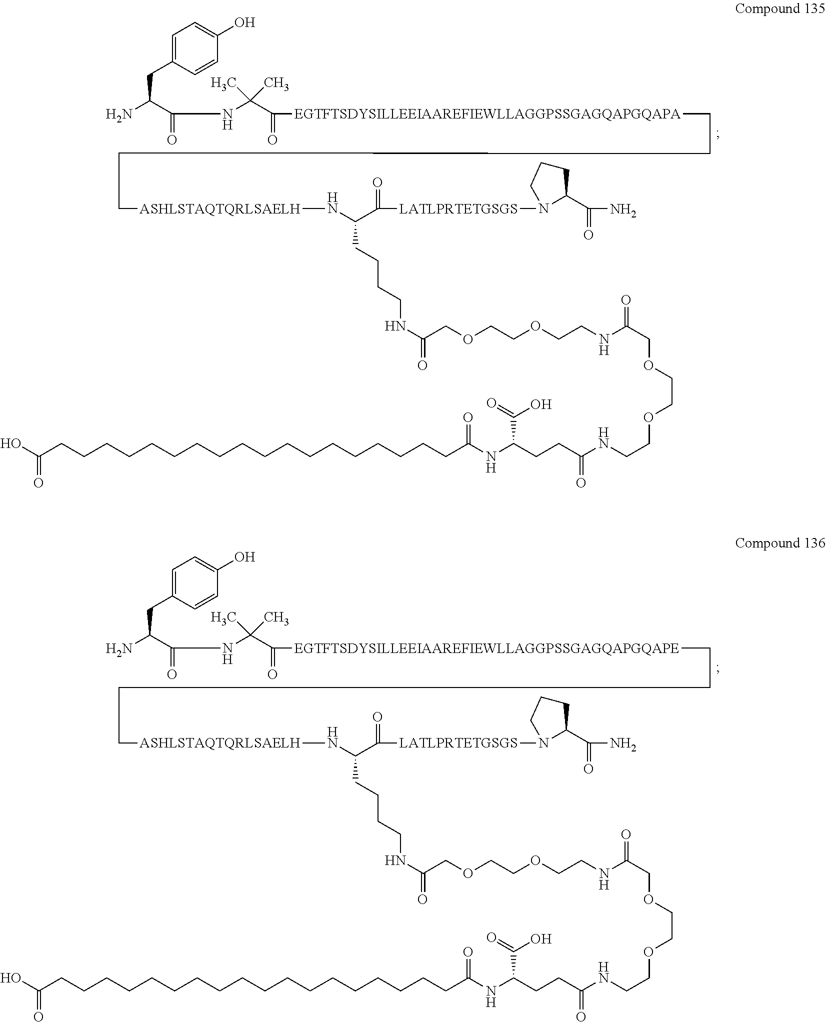

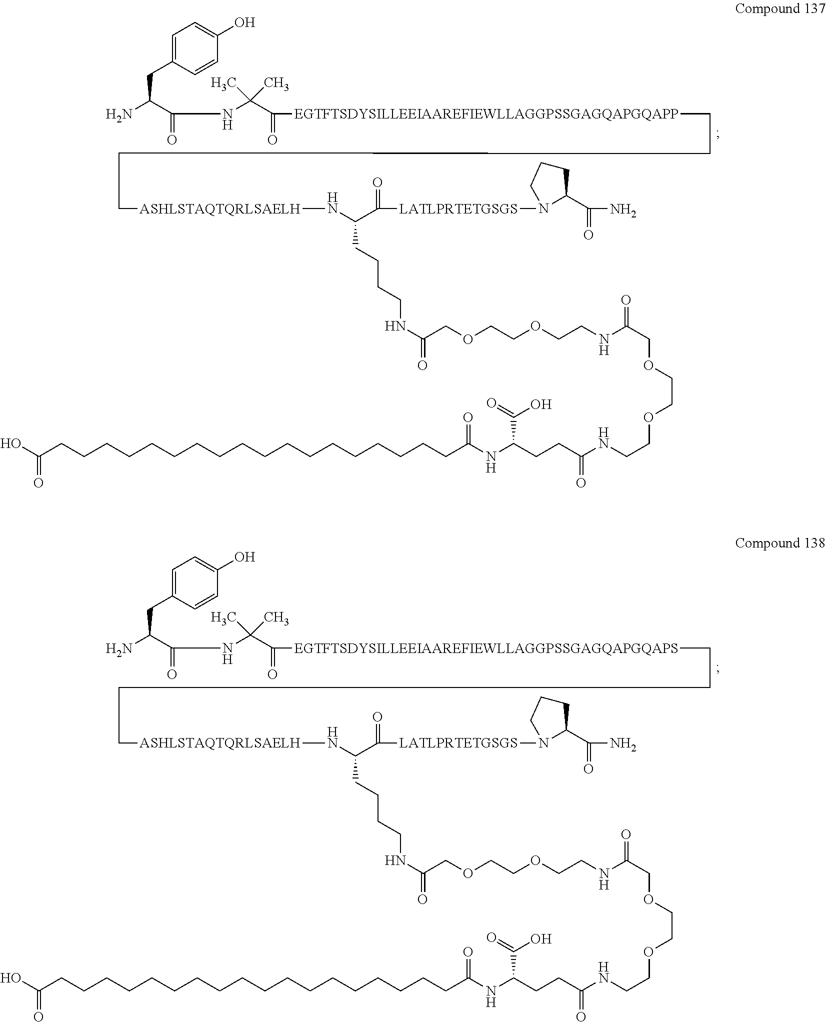

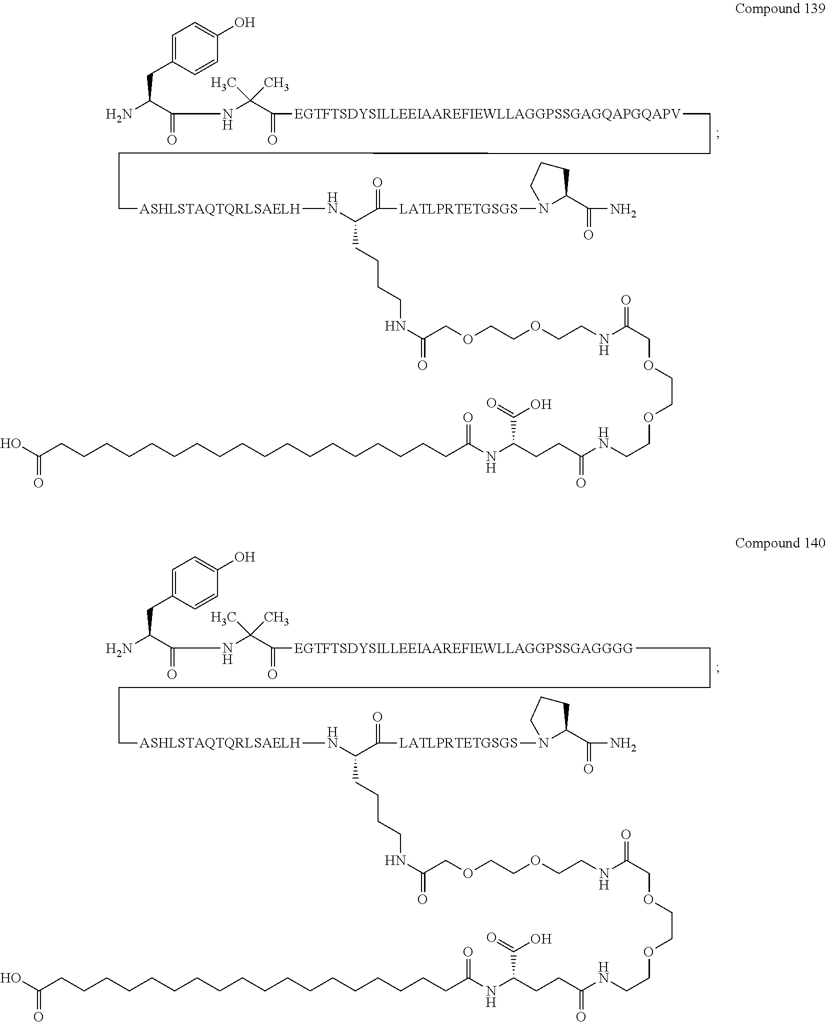

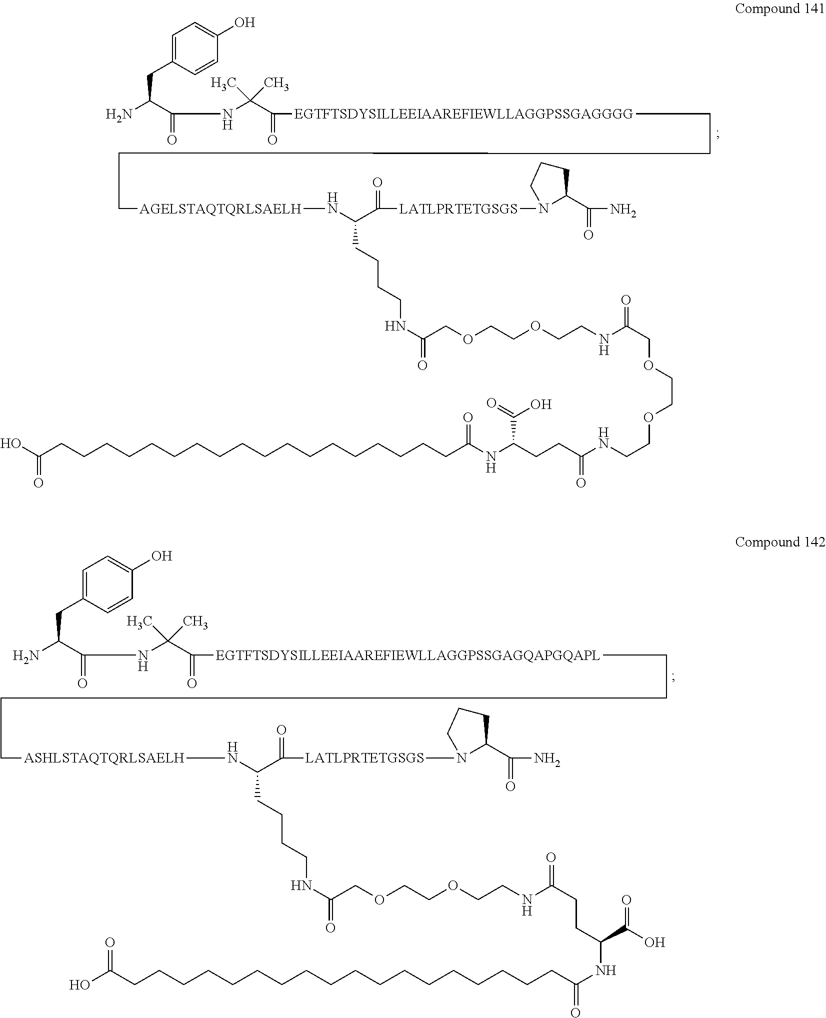

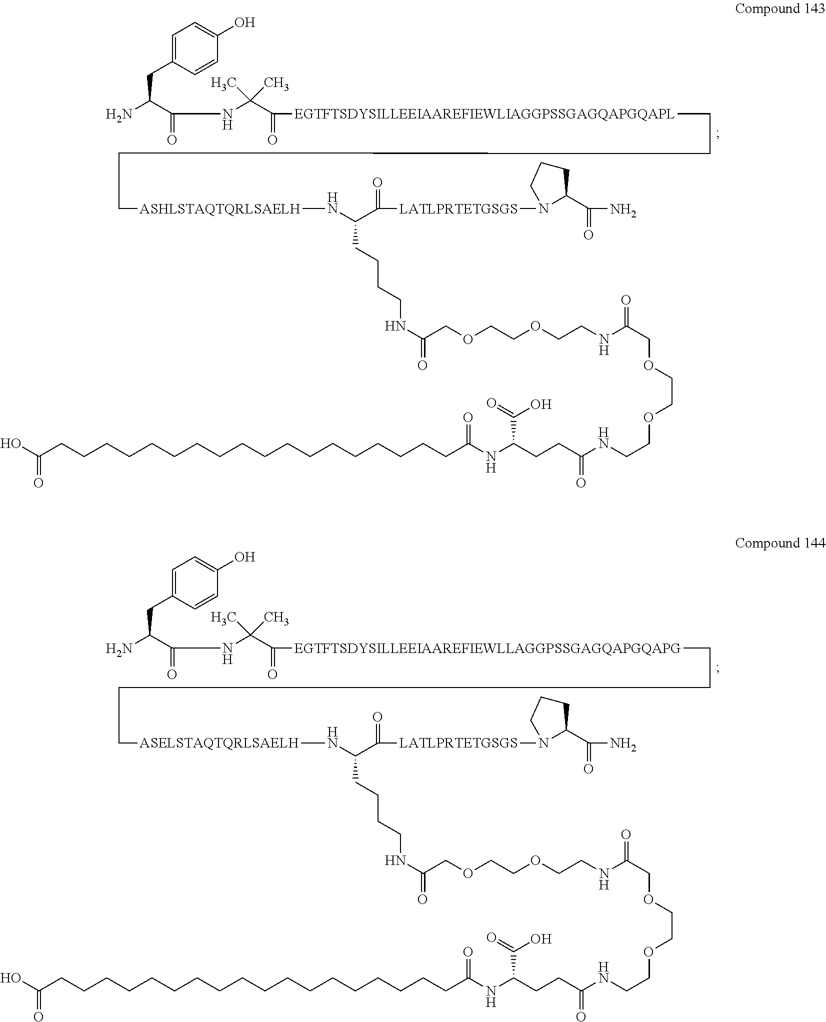

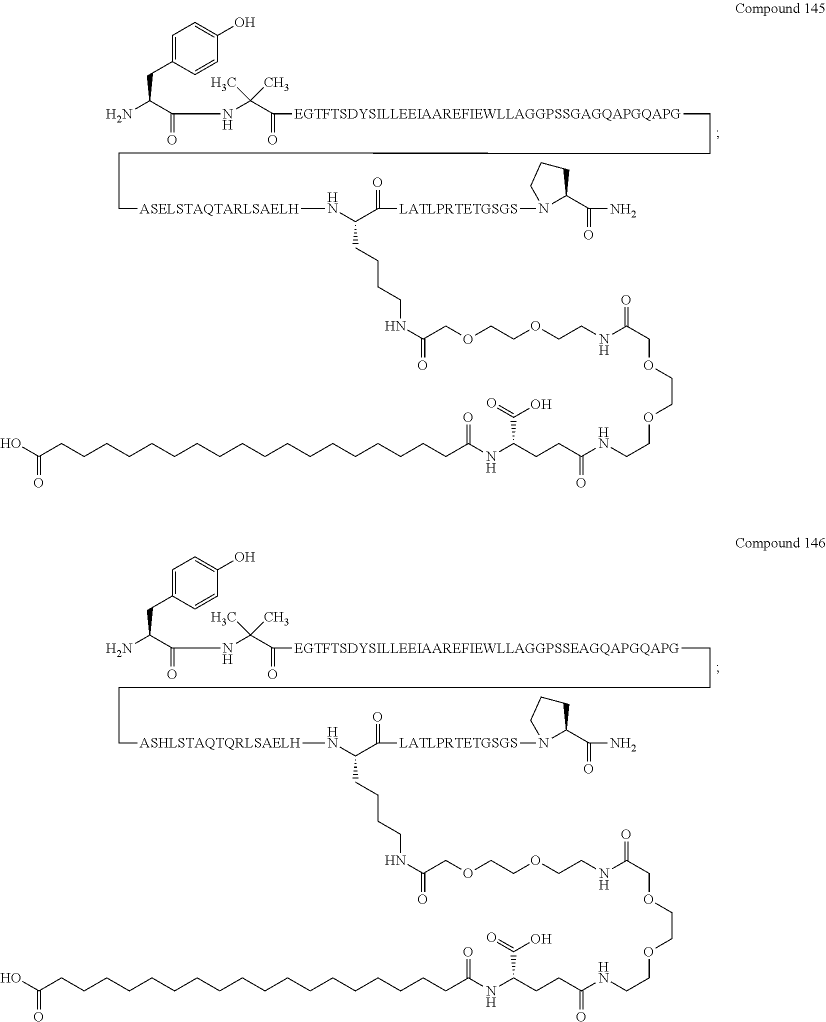

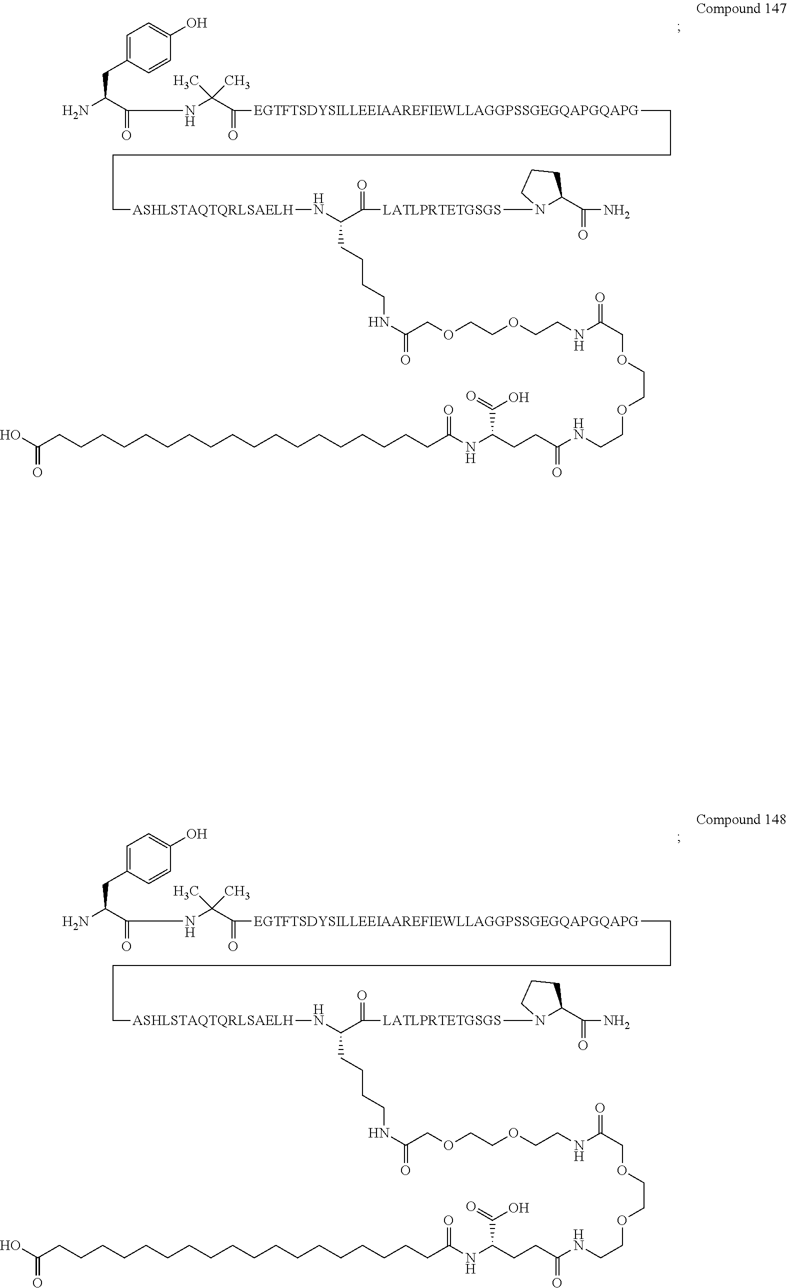

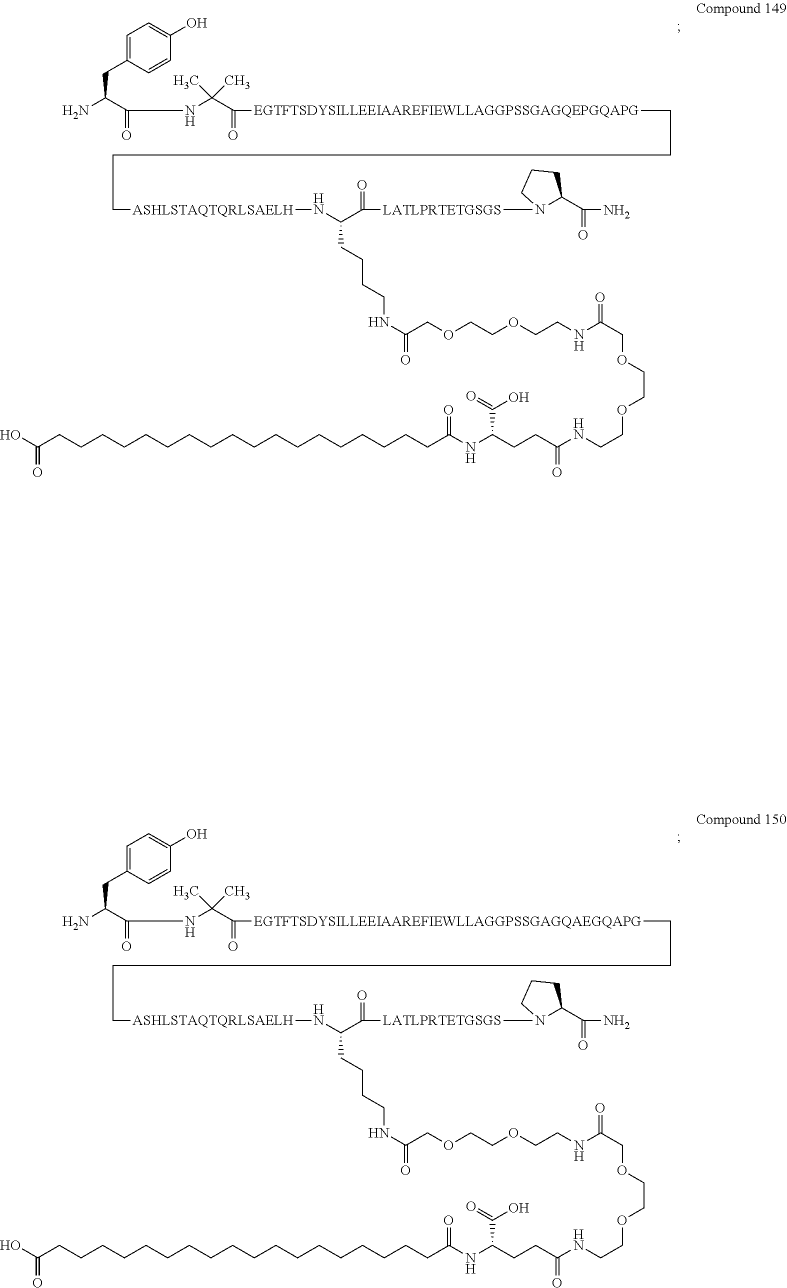

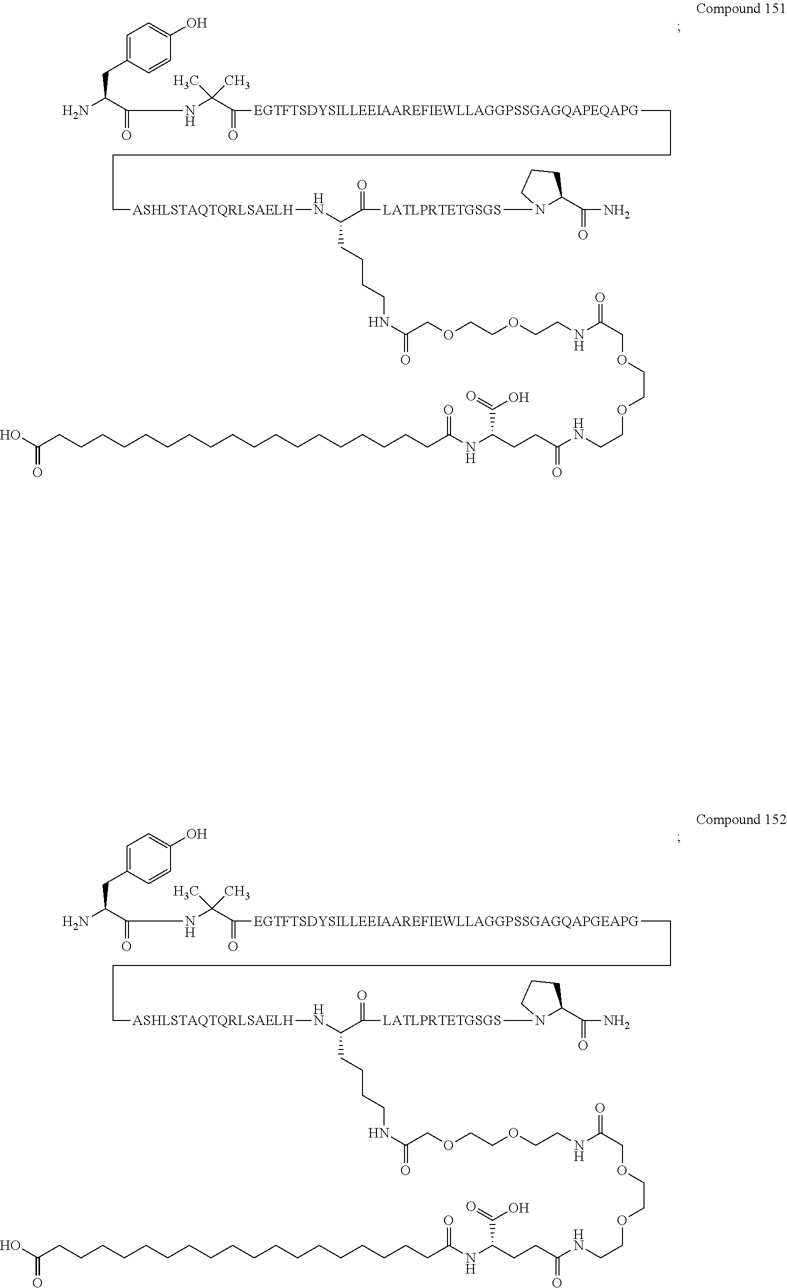

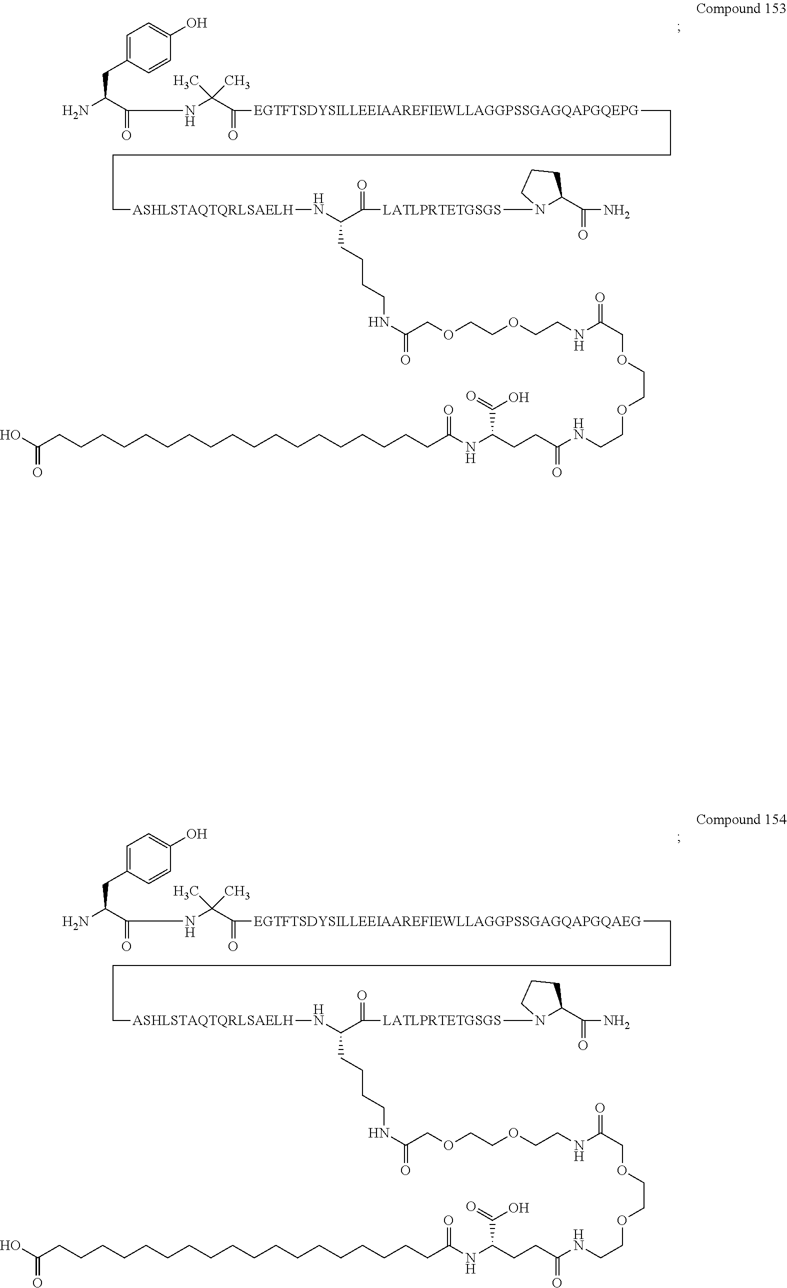

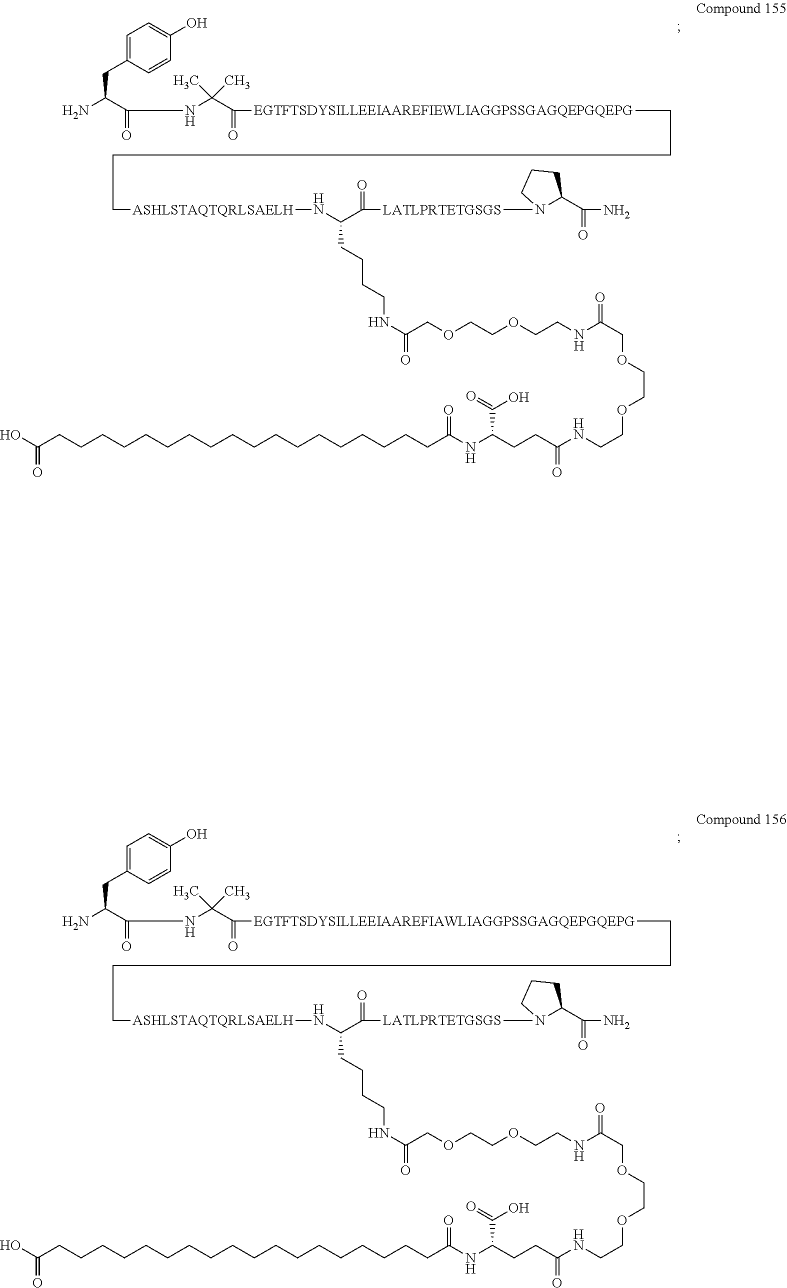

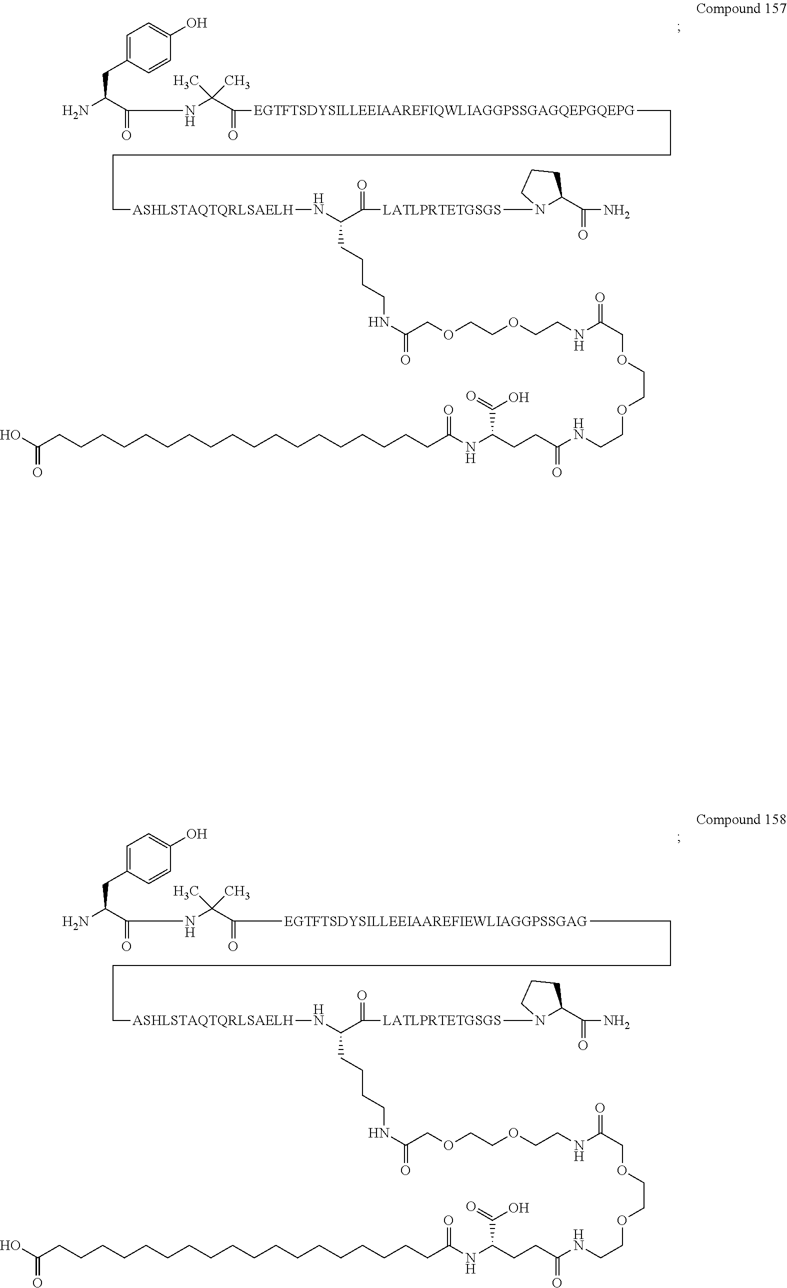

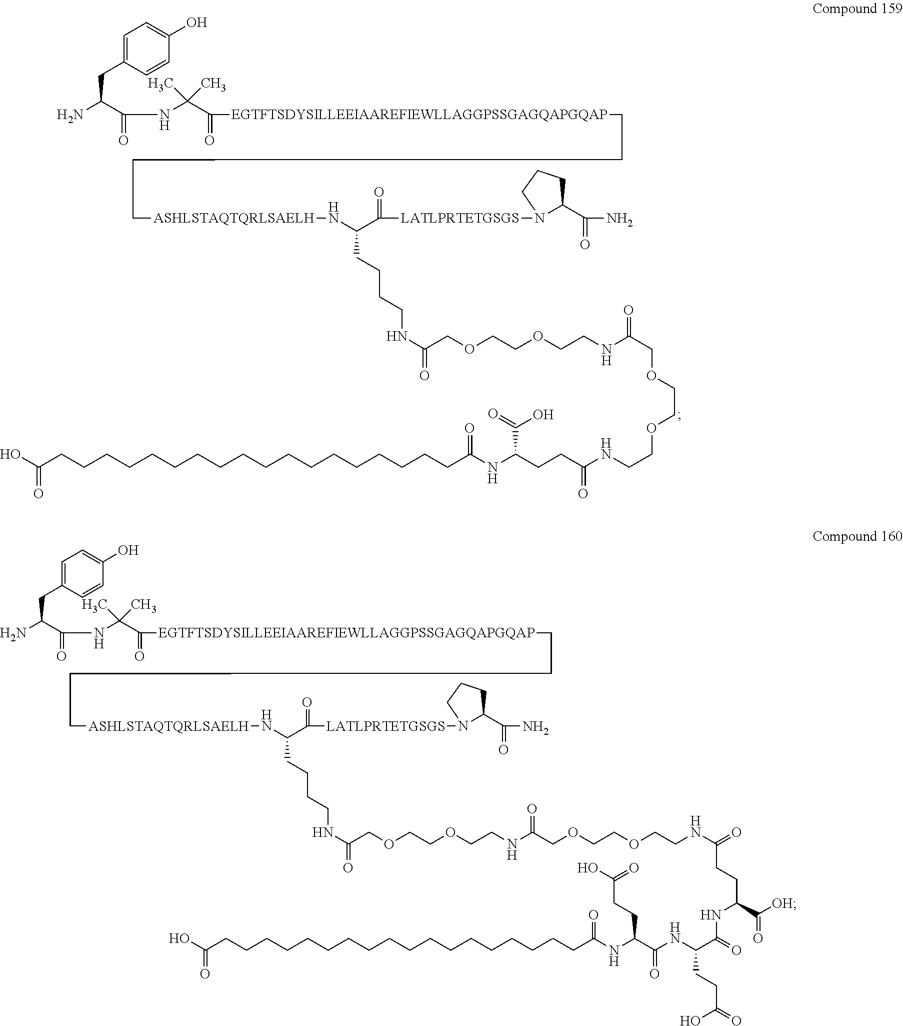

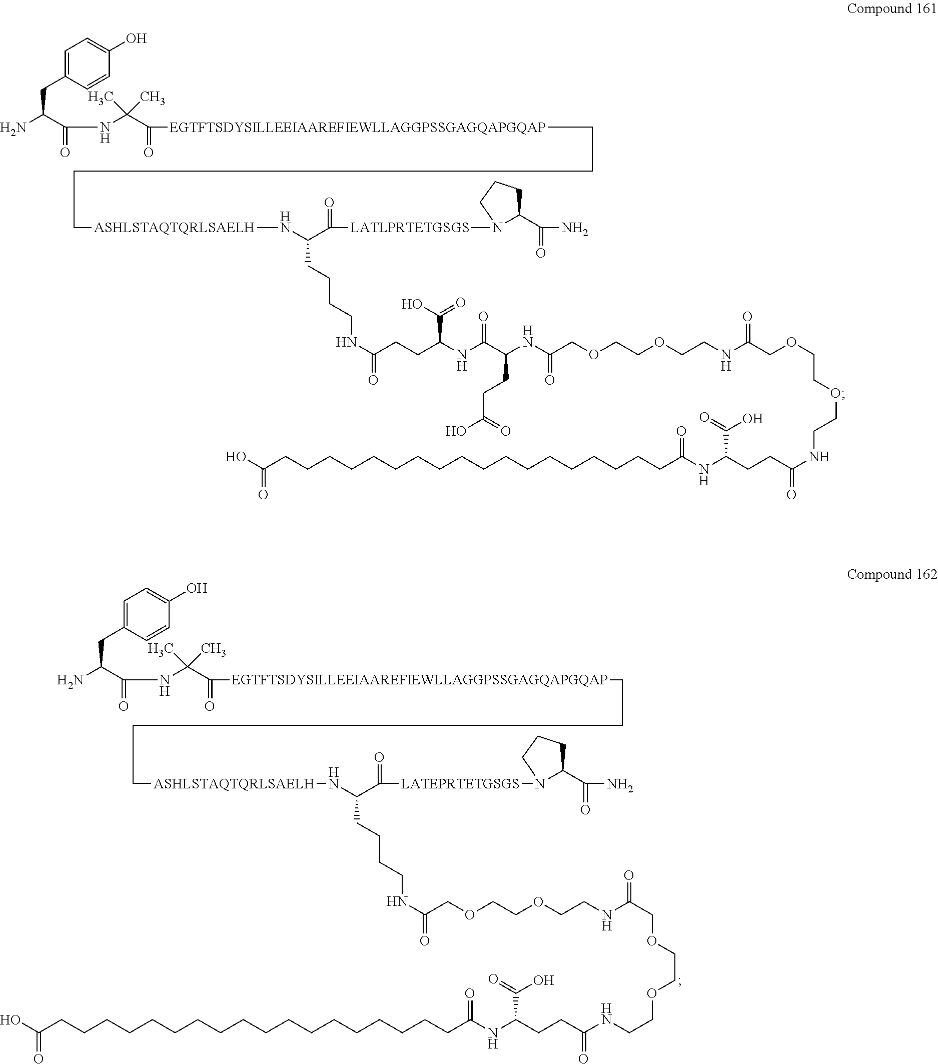

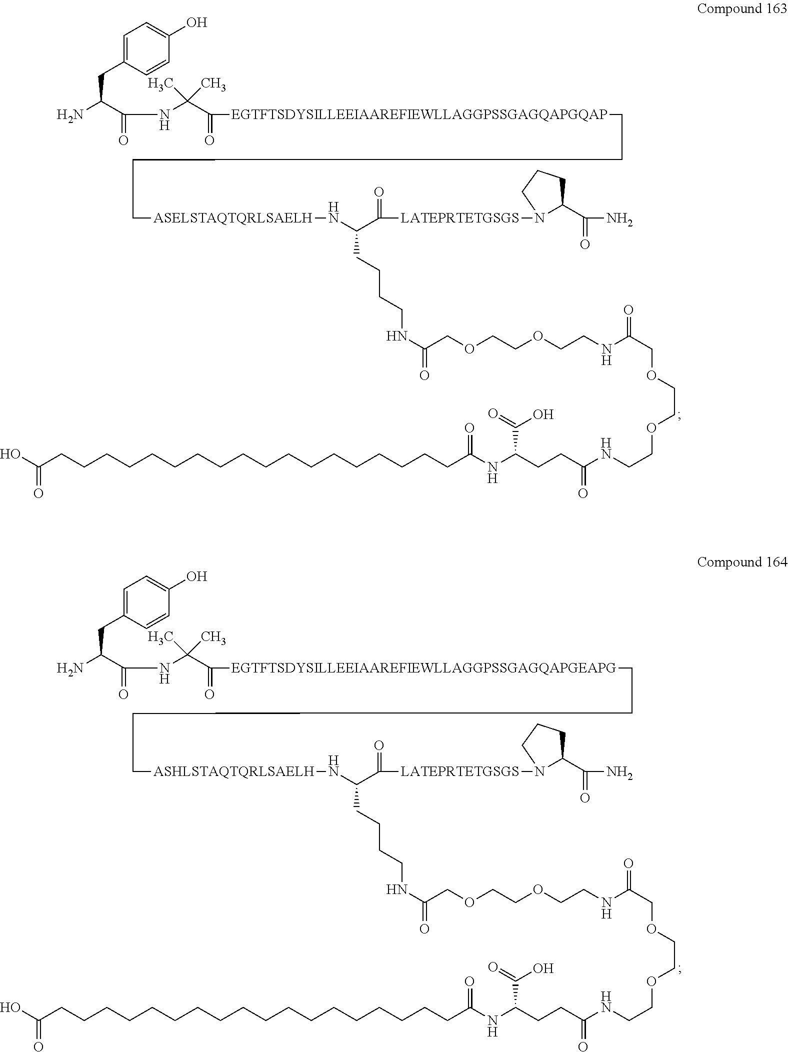

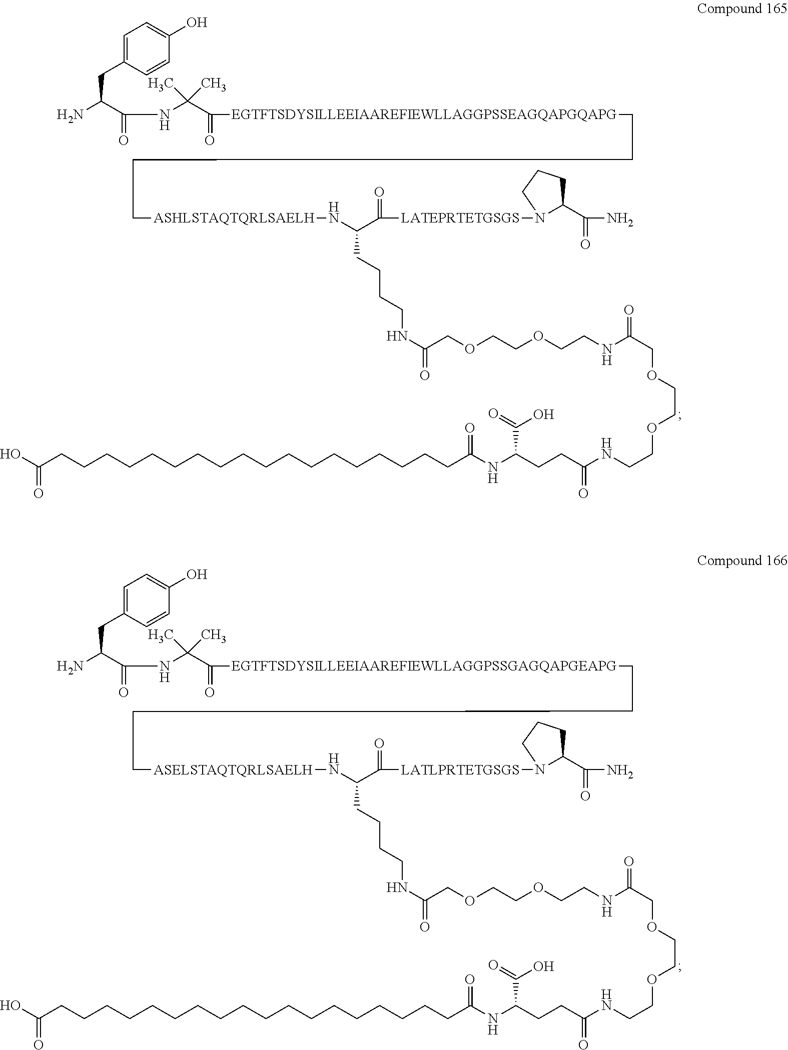

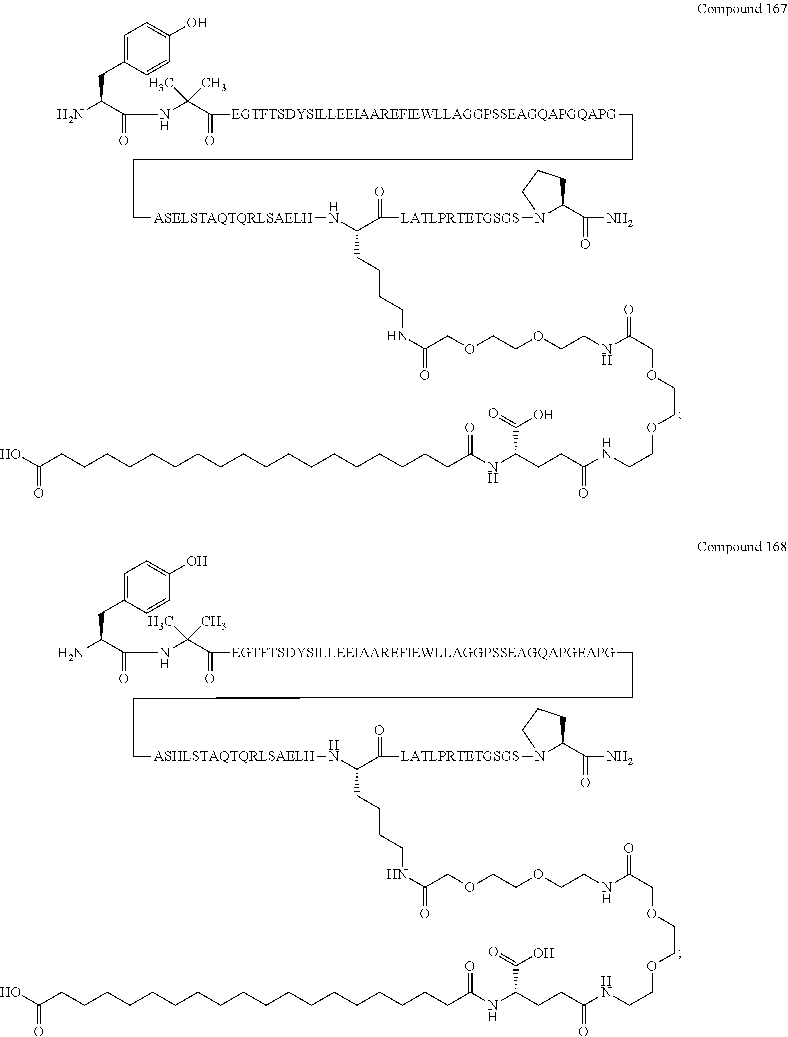

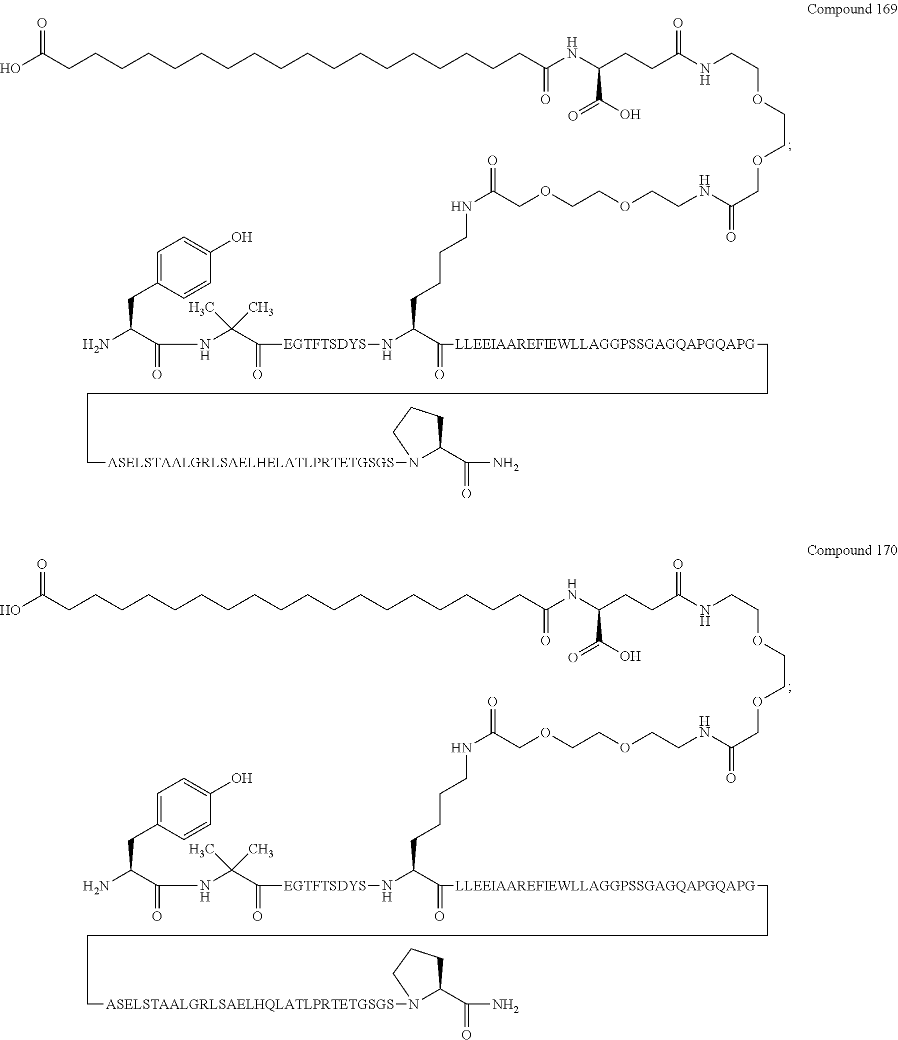

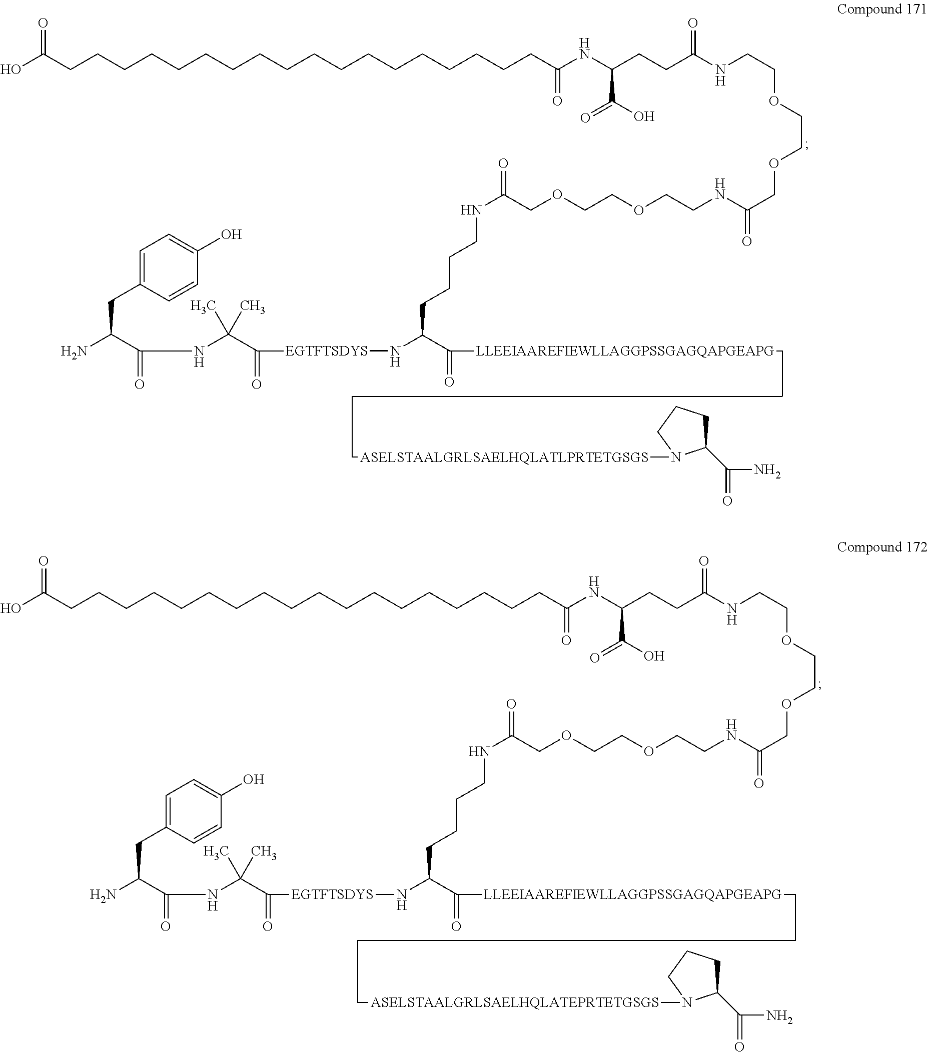

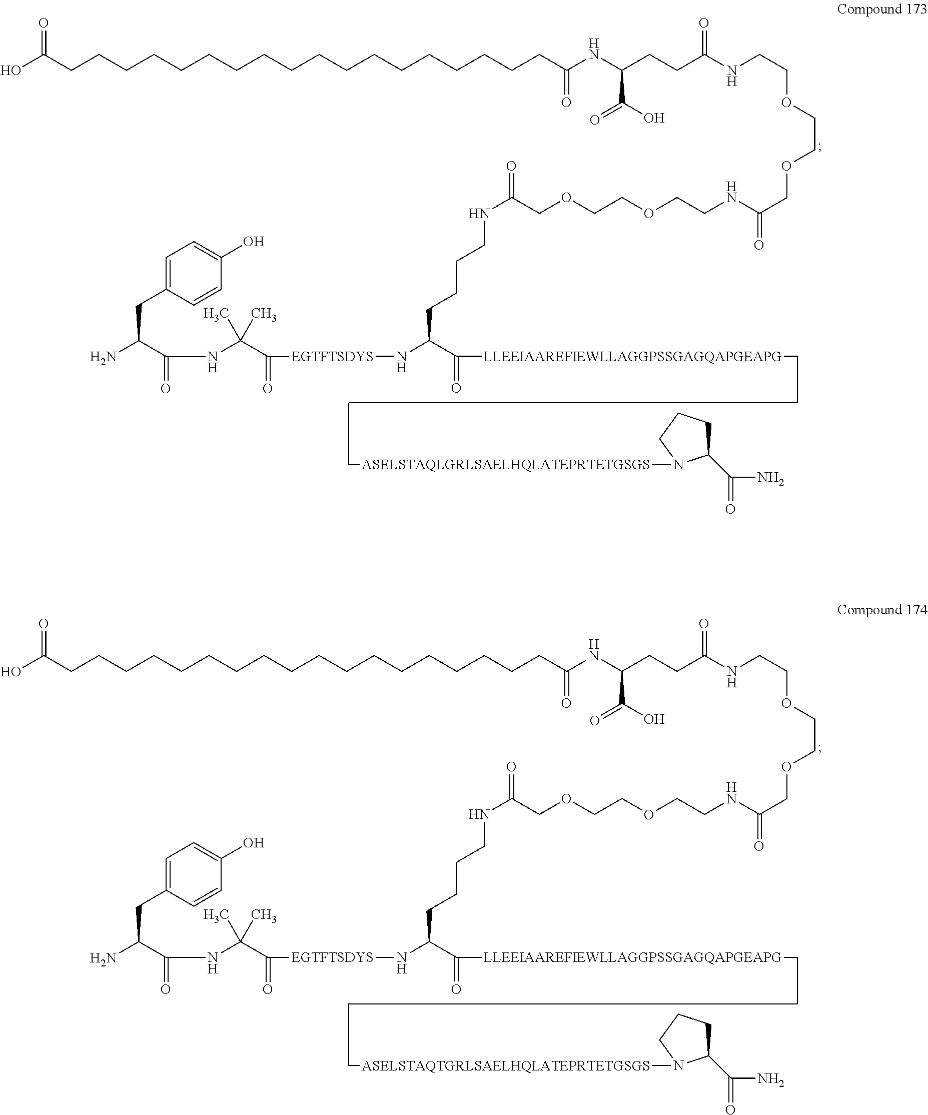

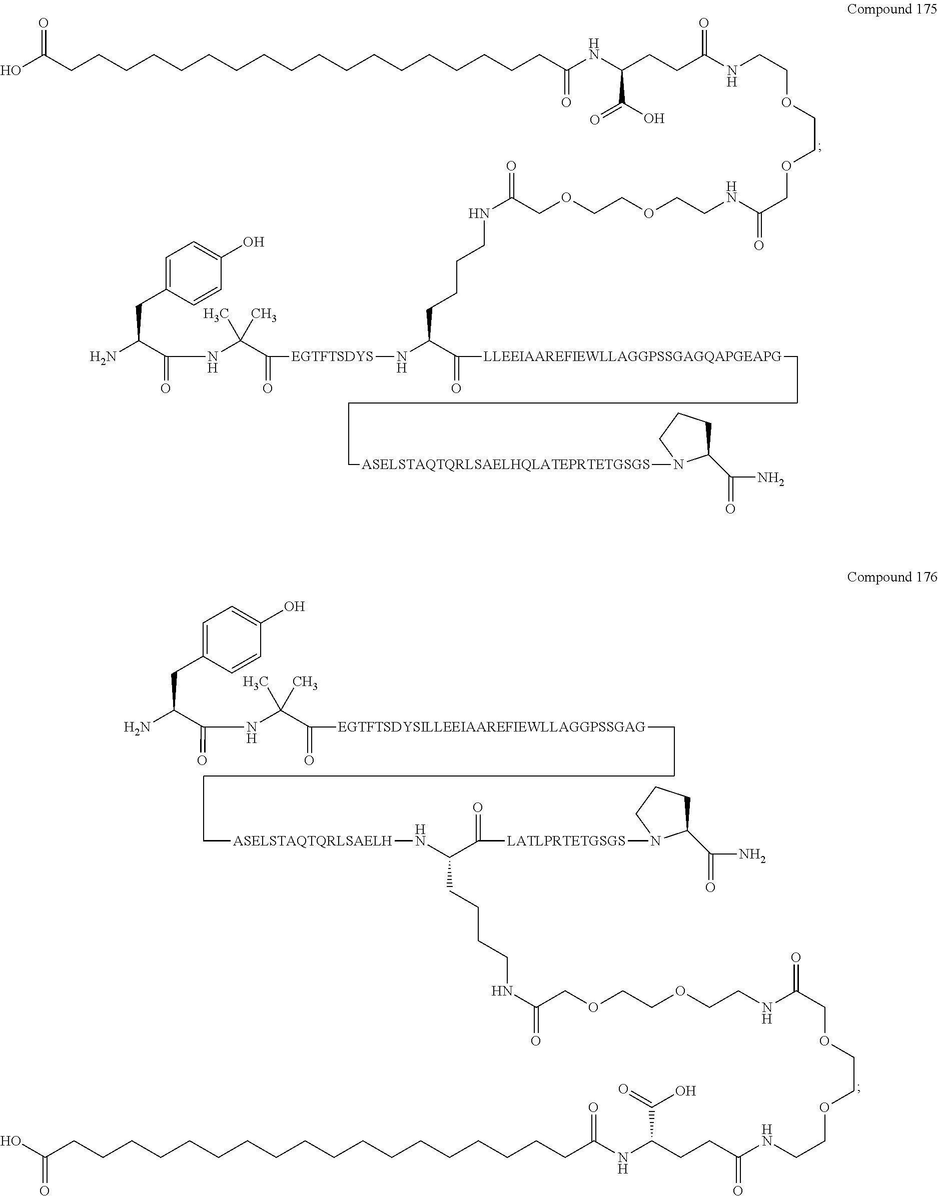

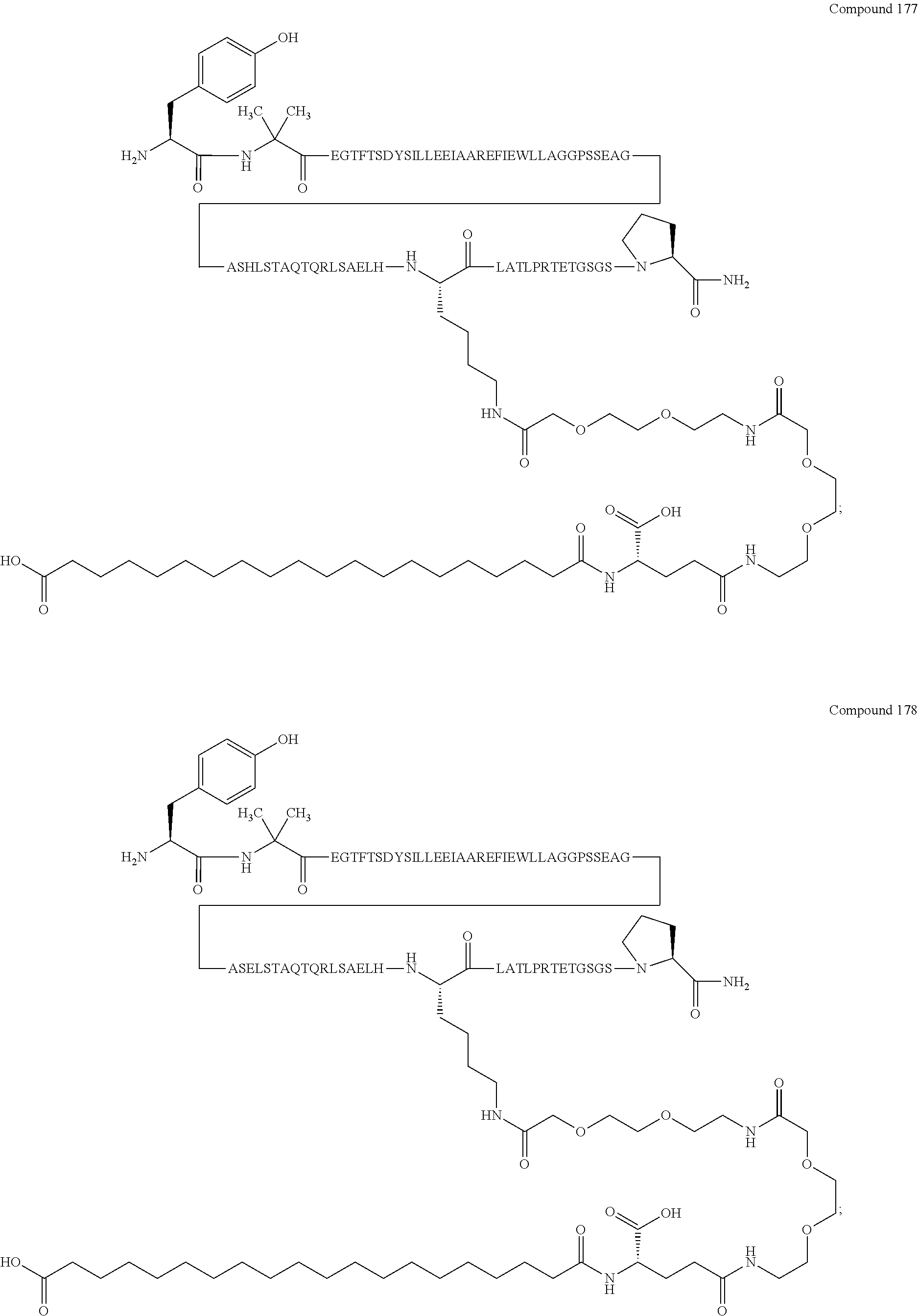

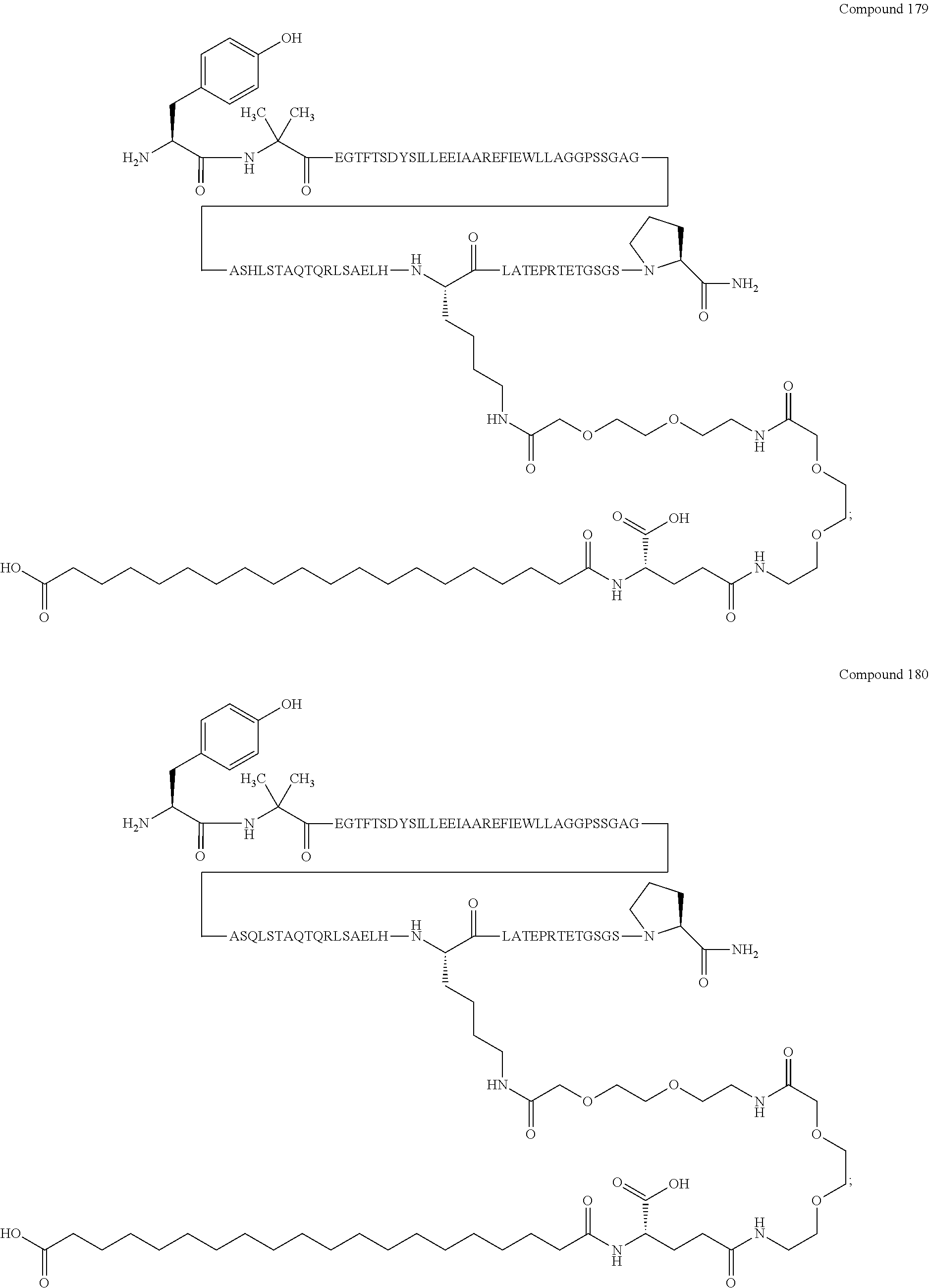

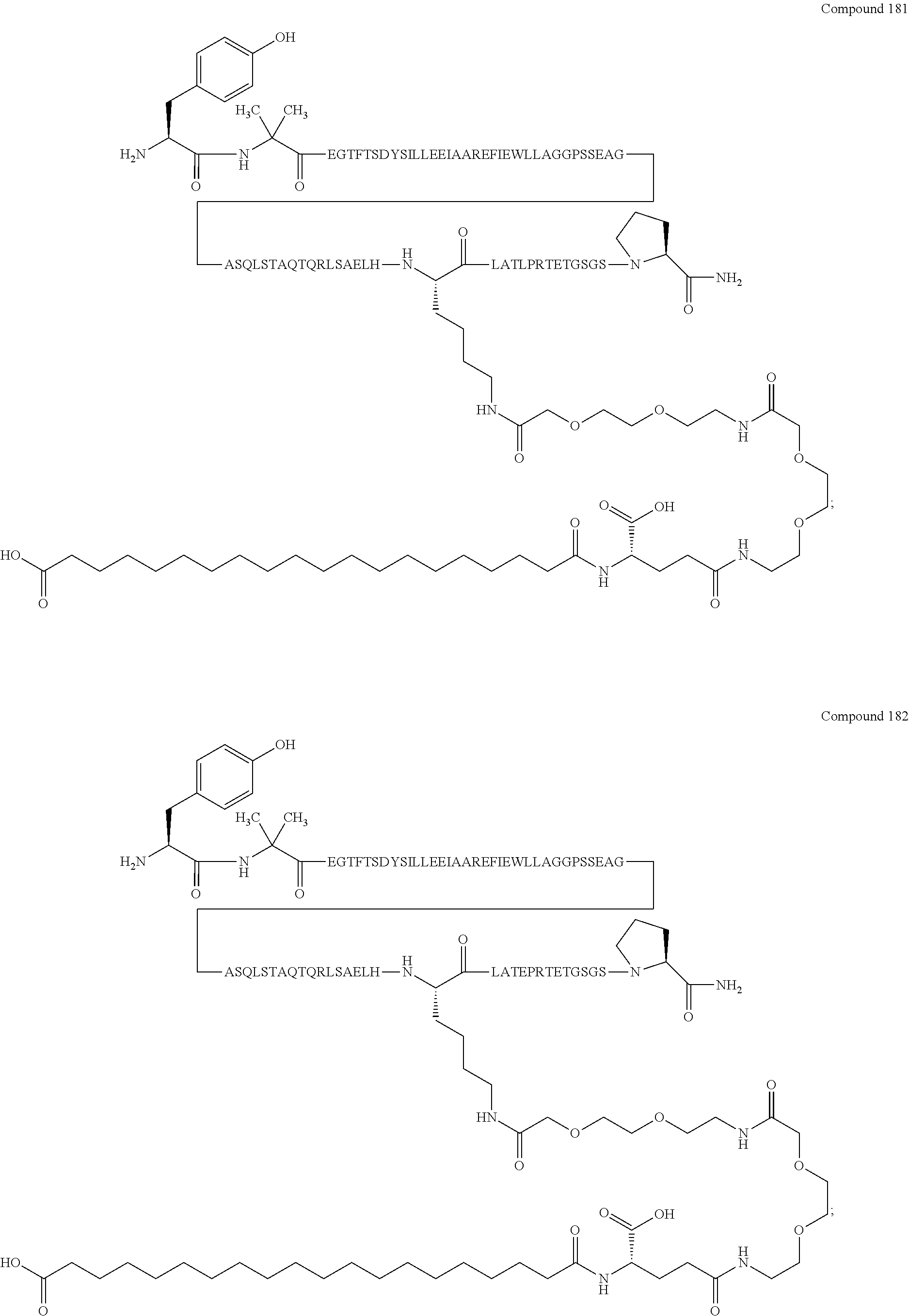

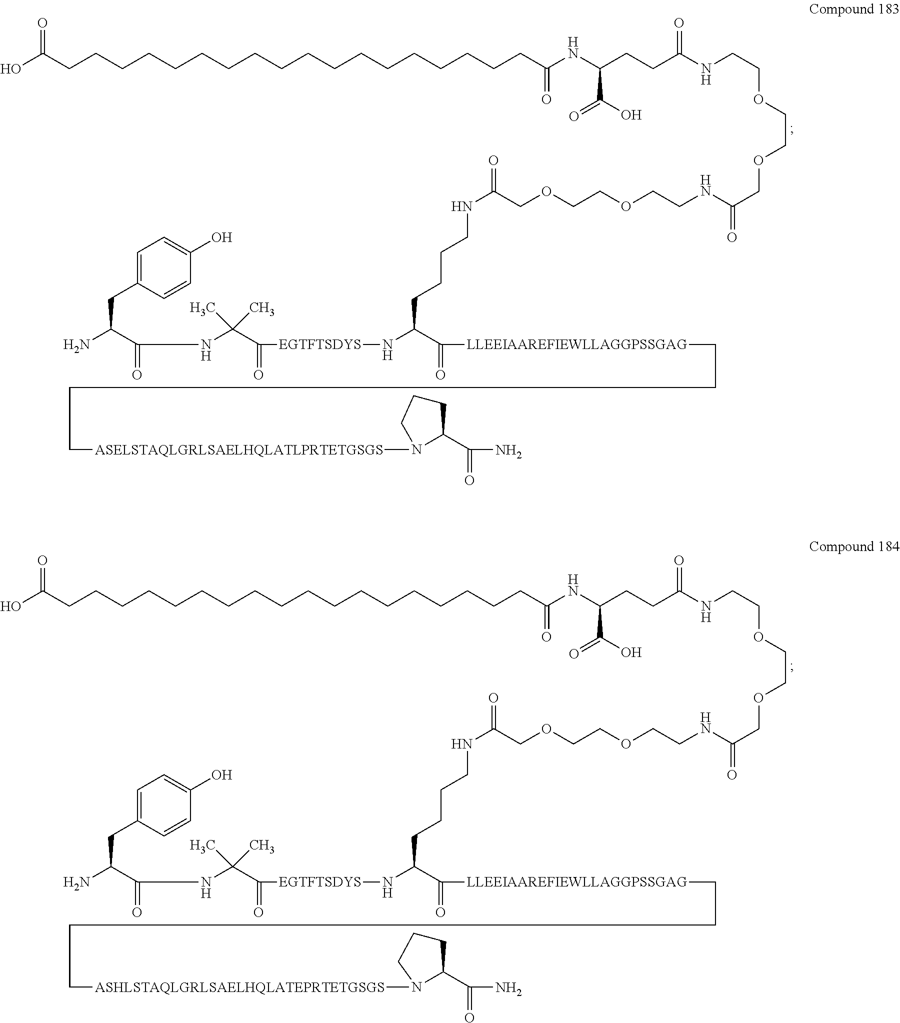

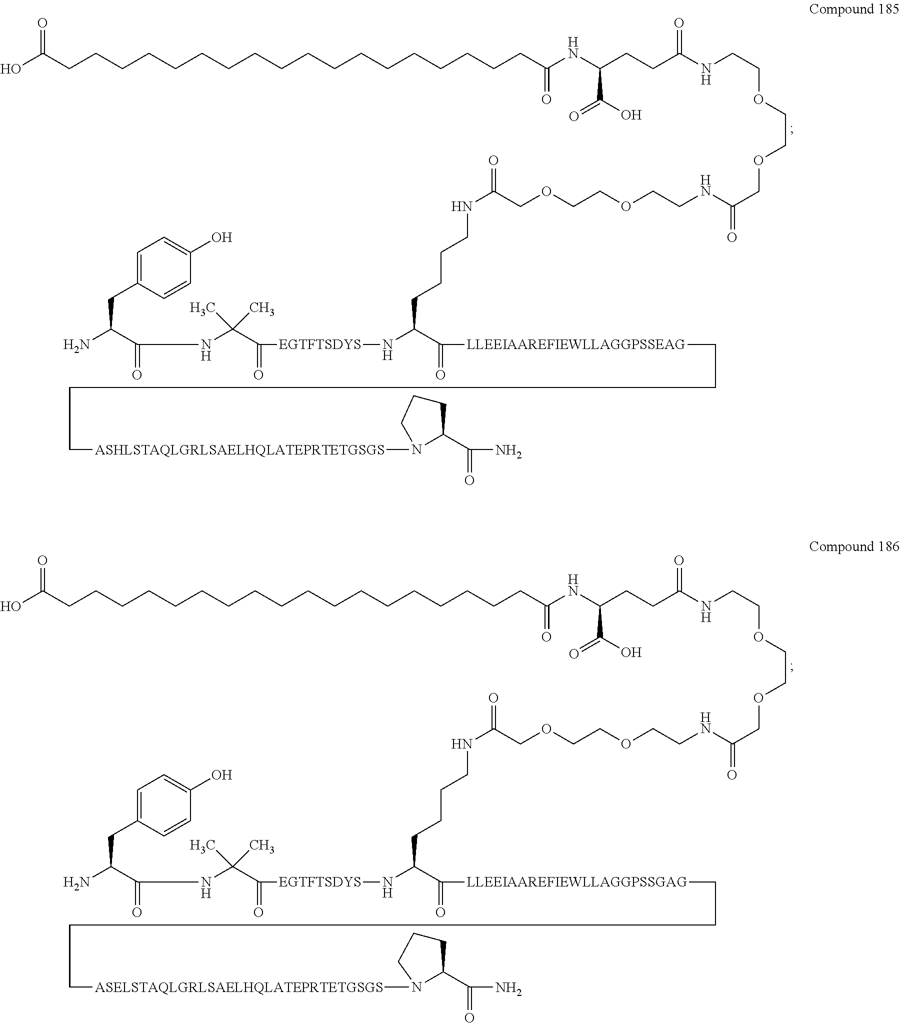

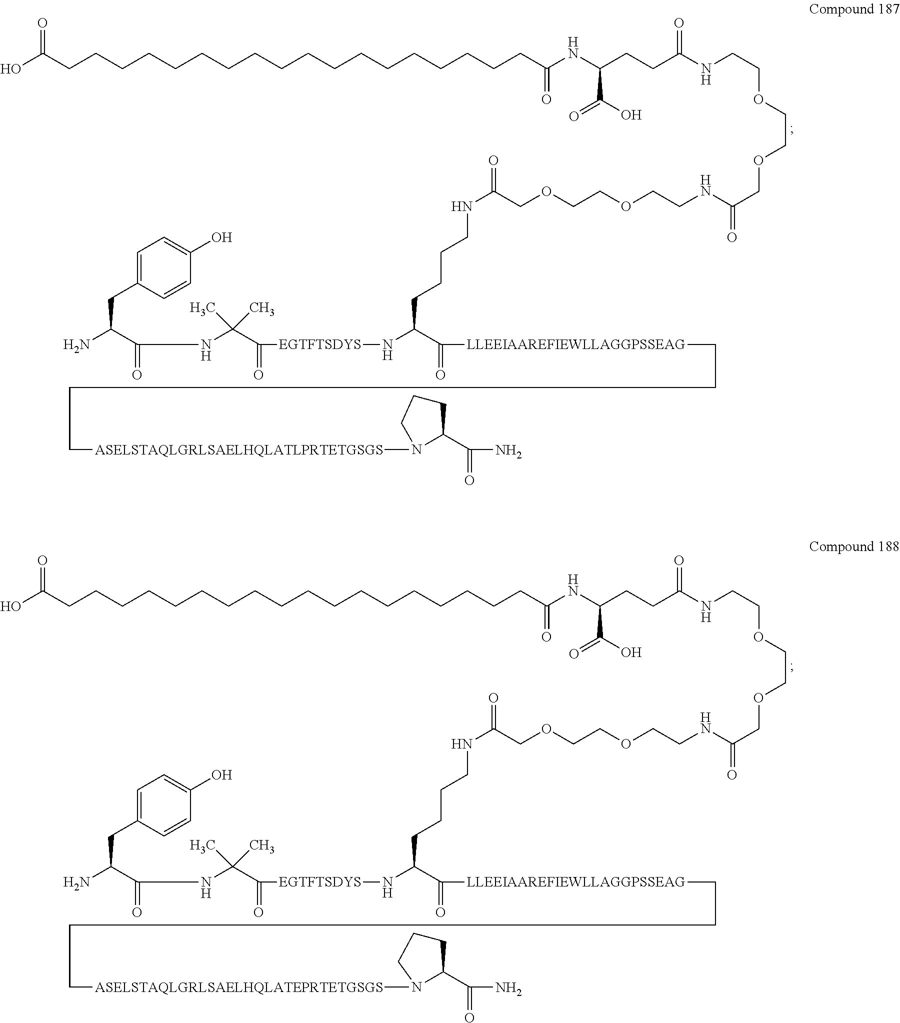

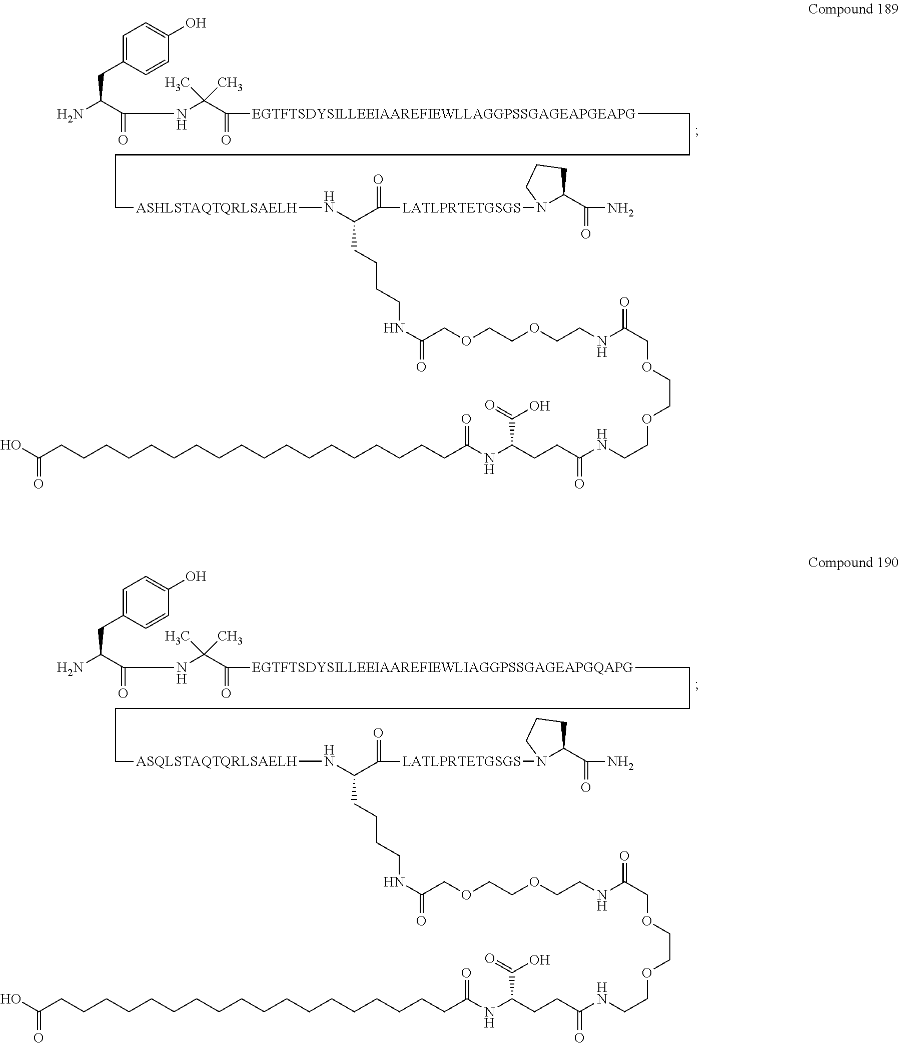

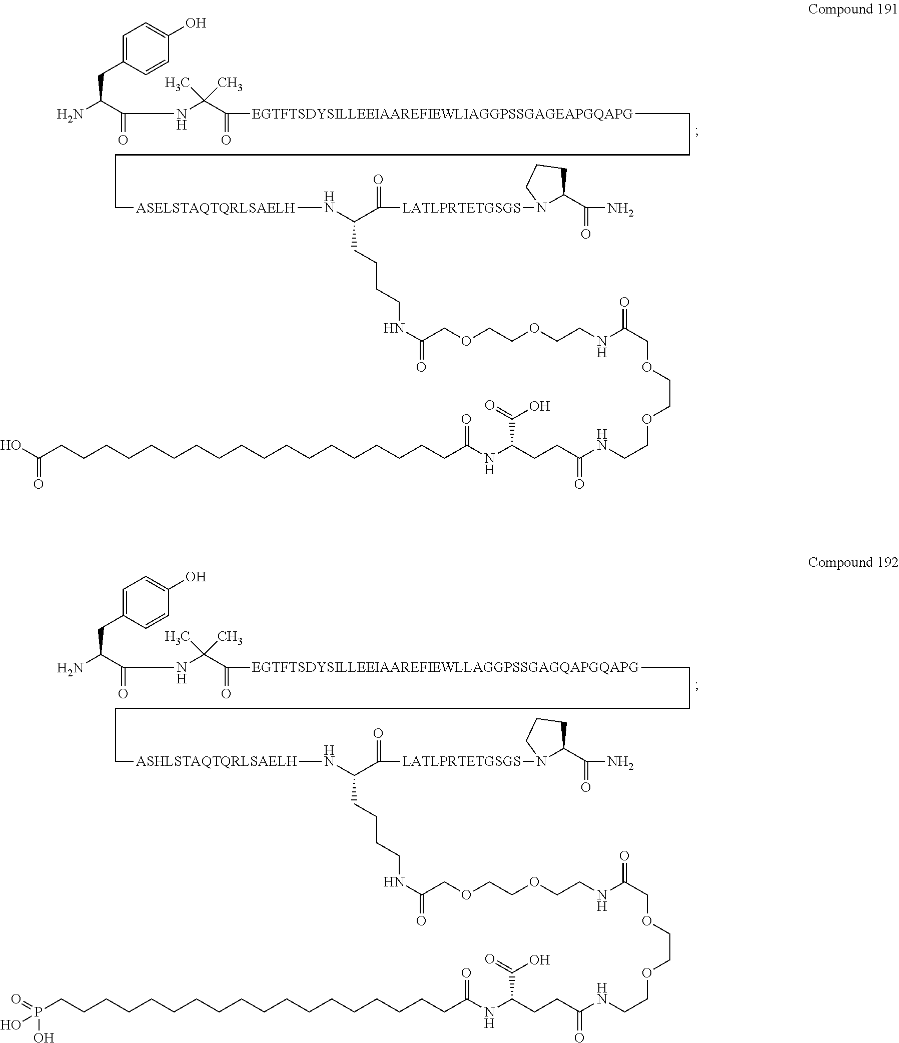

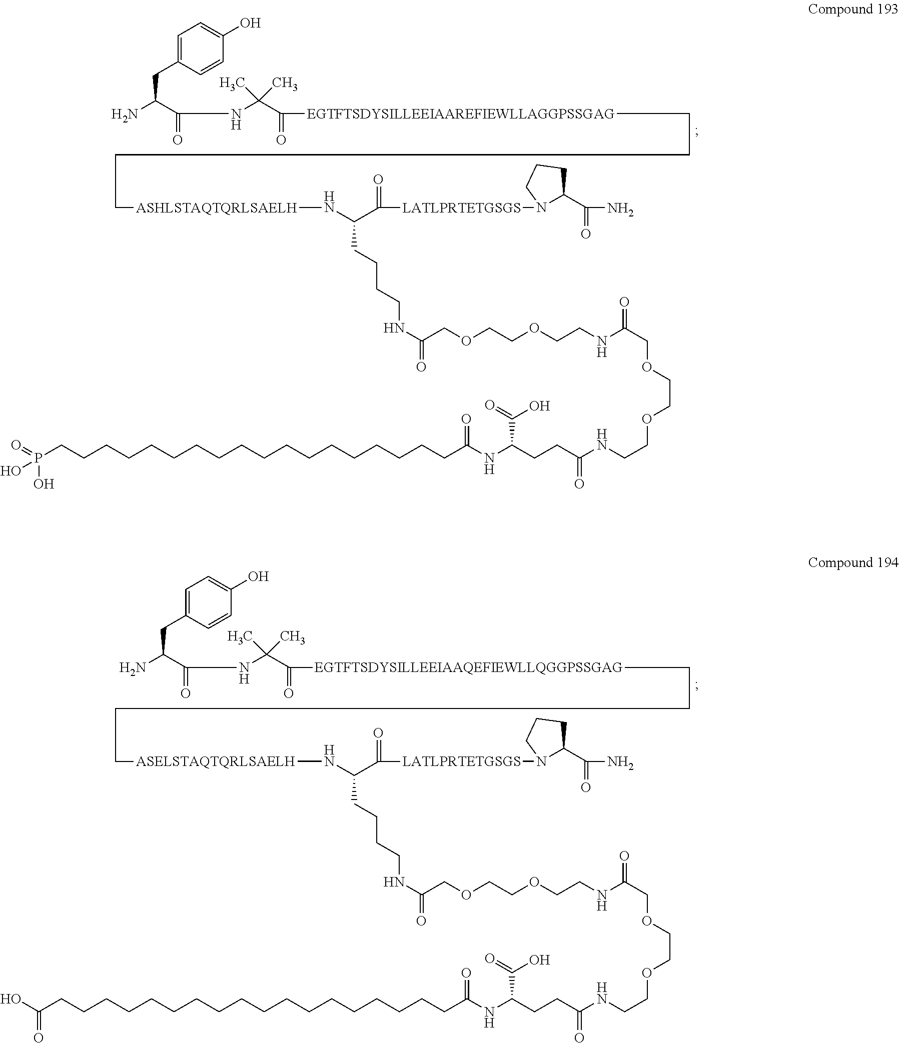

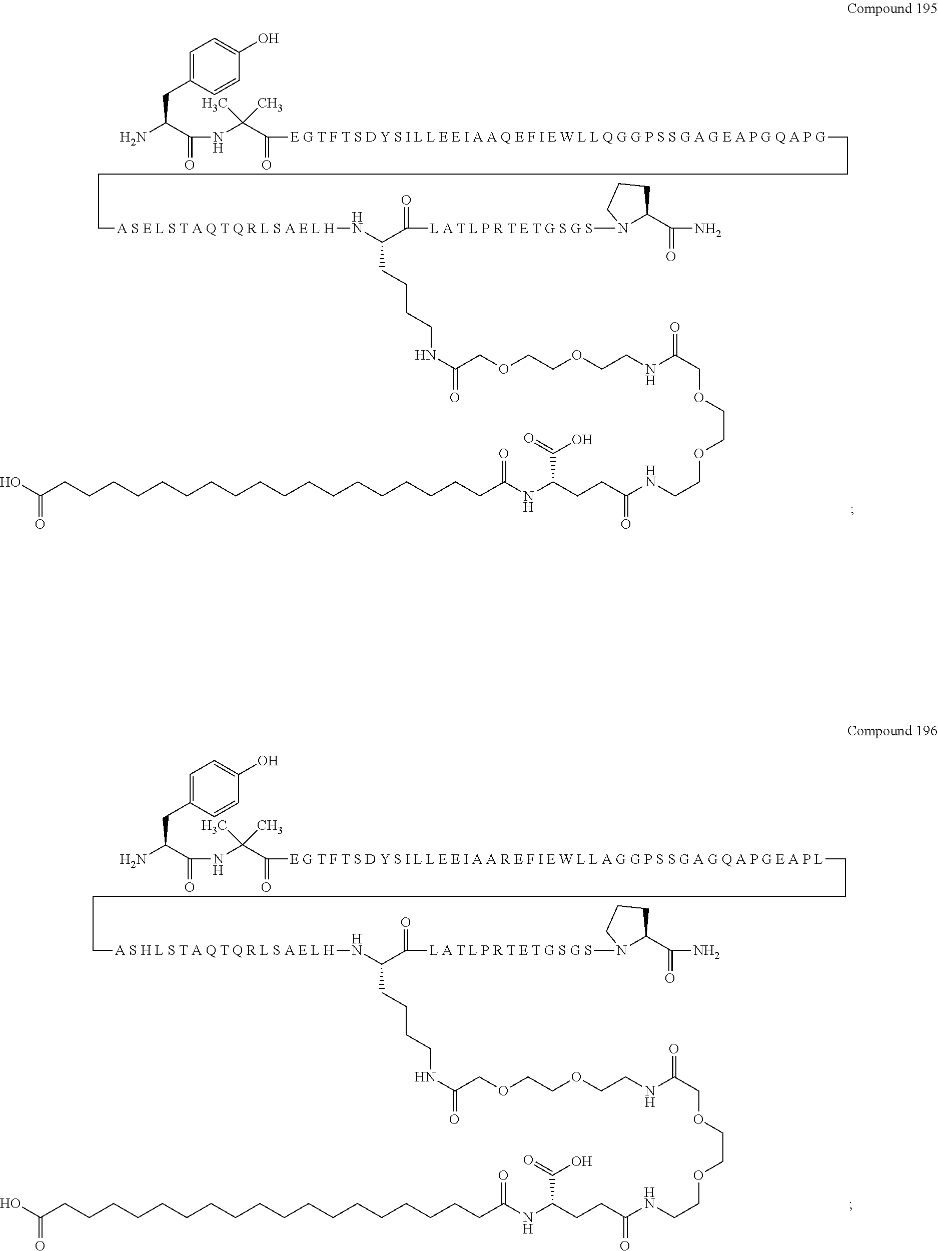

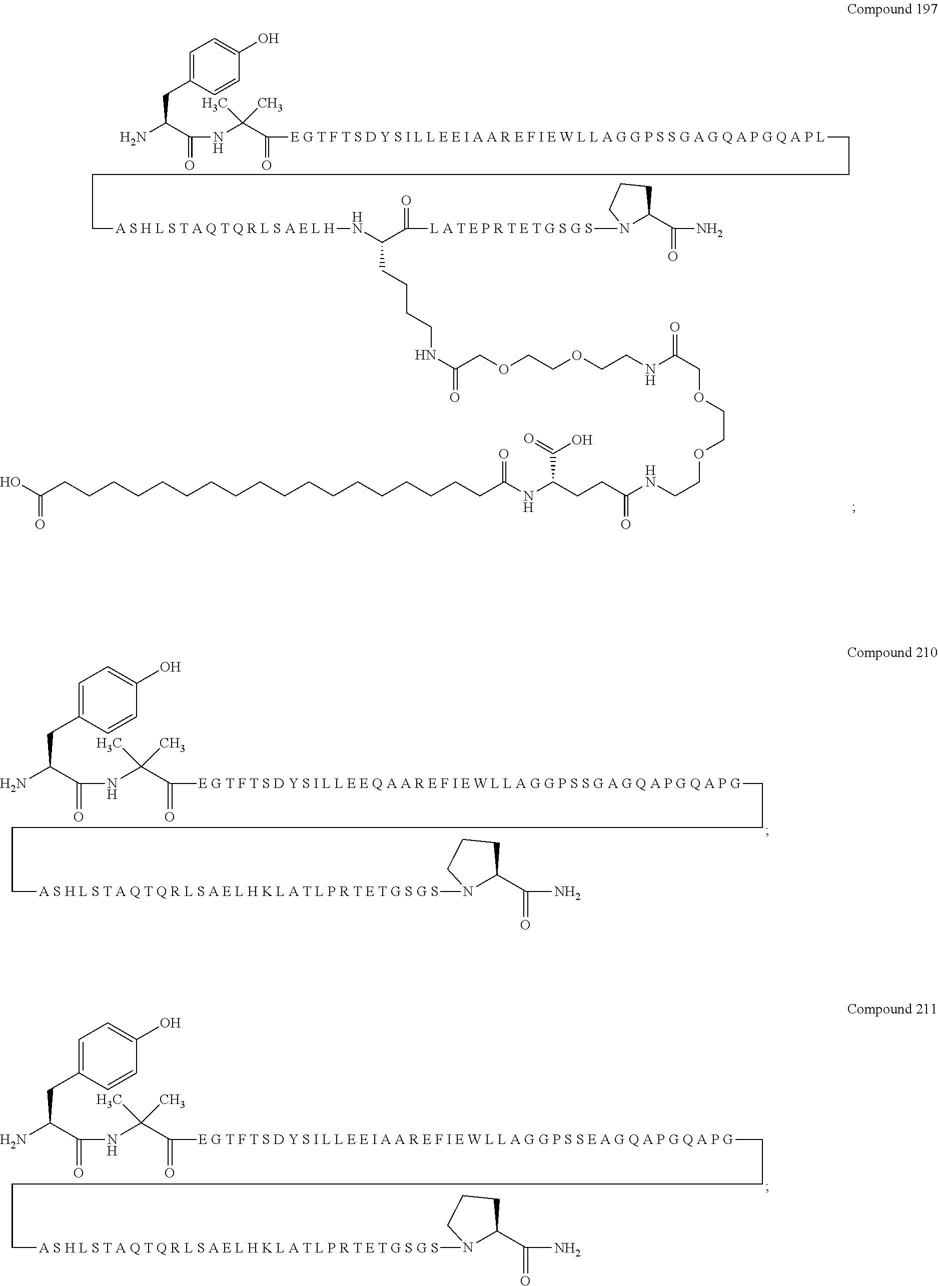

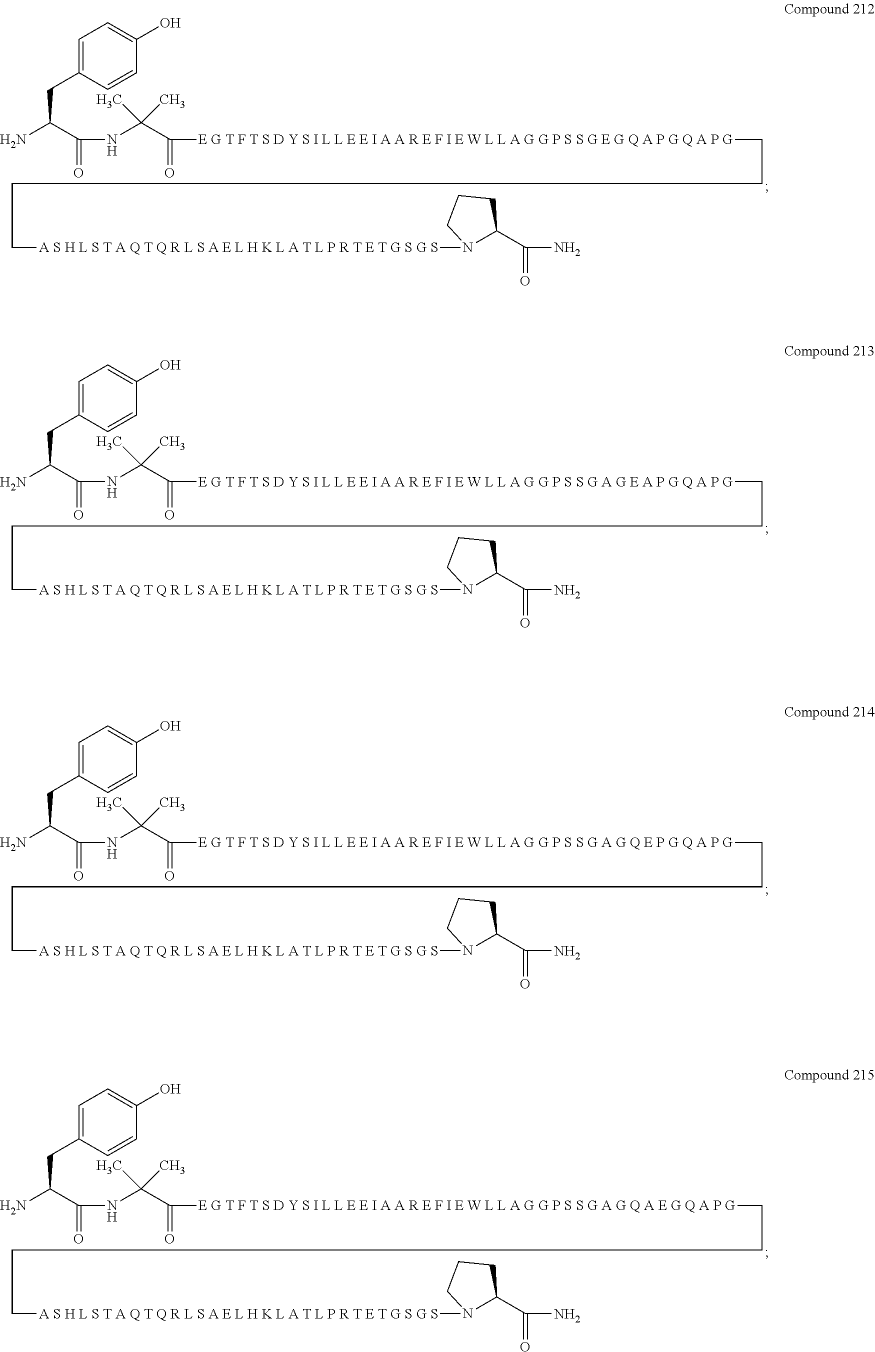

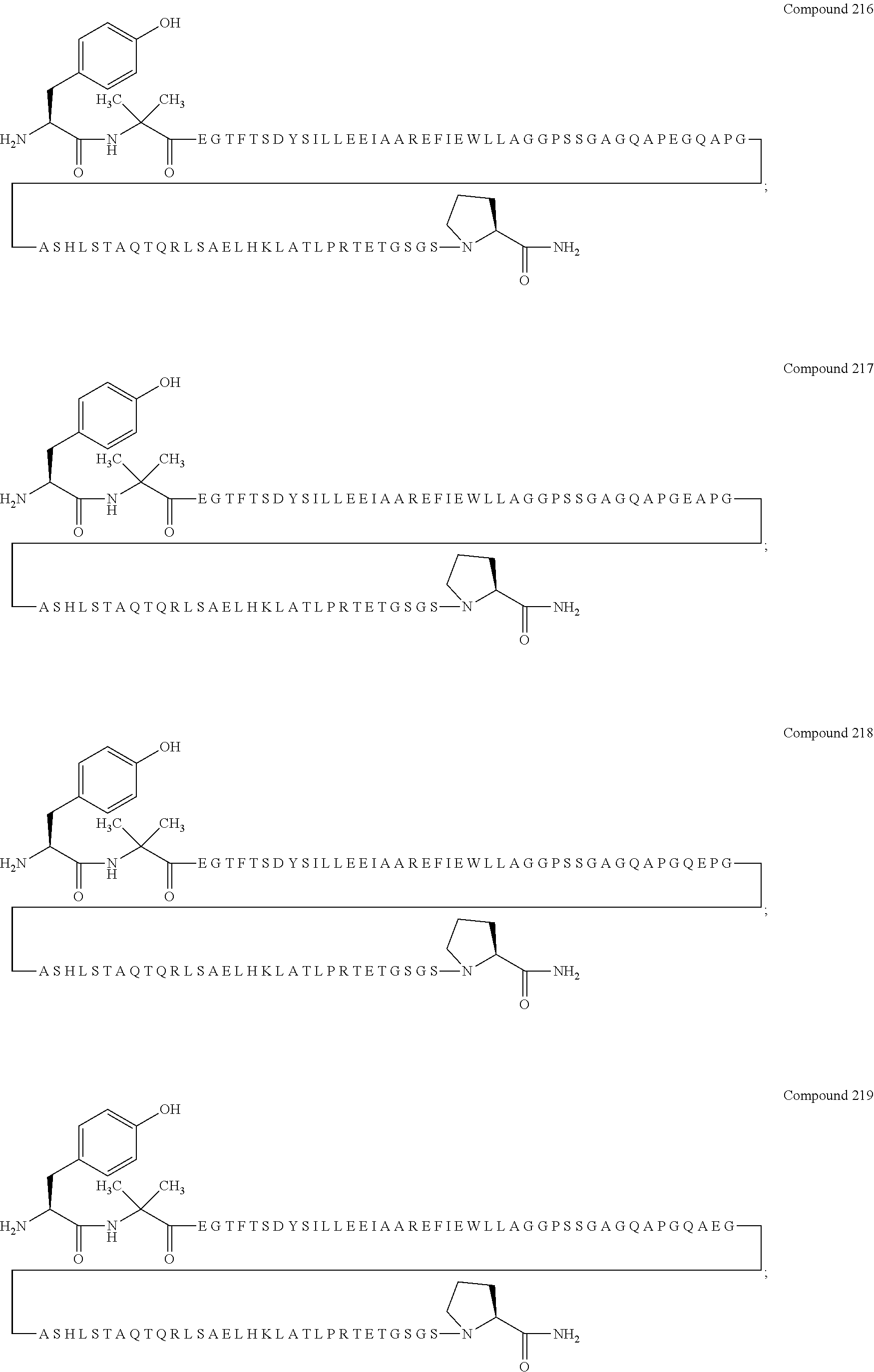

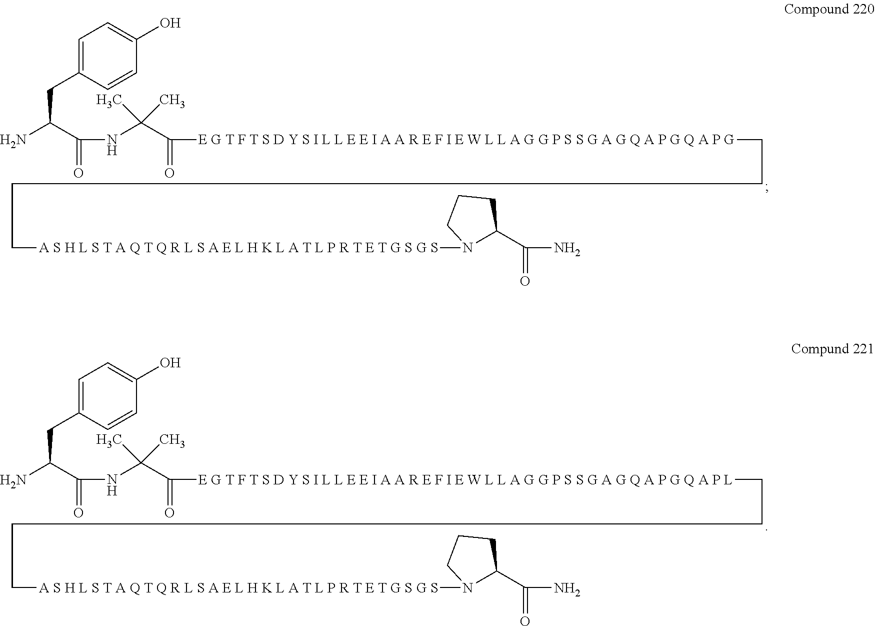

108. A GLP-1-/GIP-/amylin-receptor tri-agonist, which is compound 101

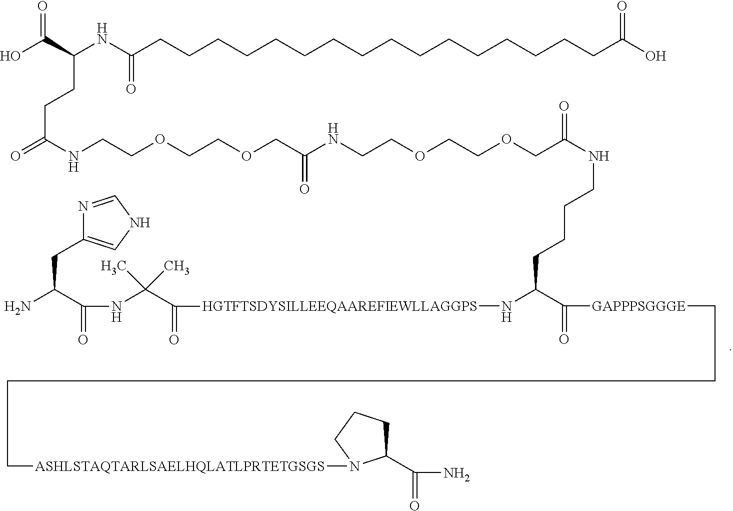

109. A GLP-1-/GIP-/amylin-receptor tri-agonist, which is compound 55

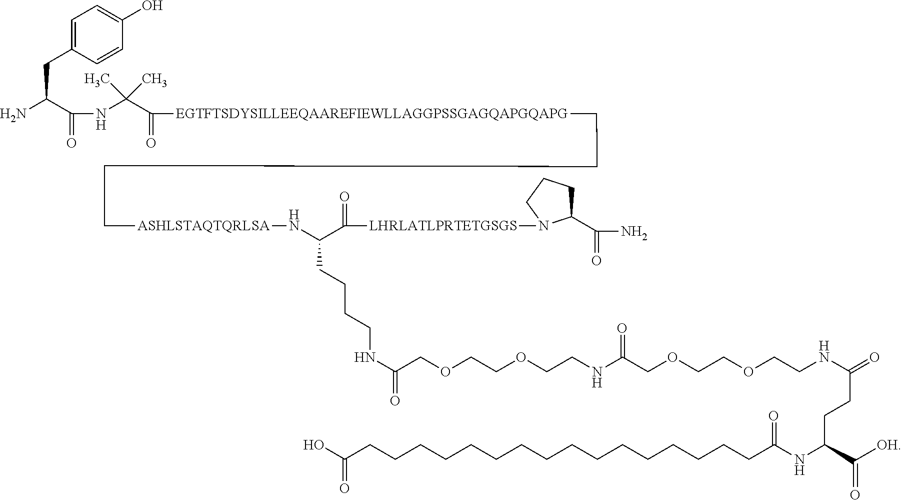

110. A GLP-1-/GIP-/amylin-receptor tri-agonist, which is compound 52

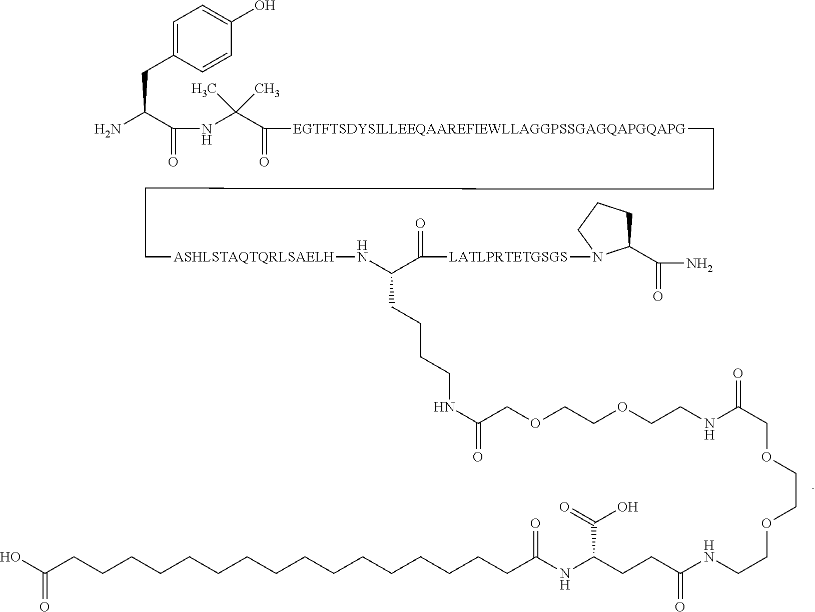

111. A GLP-1-/GIP-/amylin-receptor tri-agonist, which is compound 58

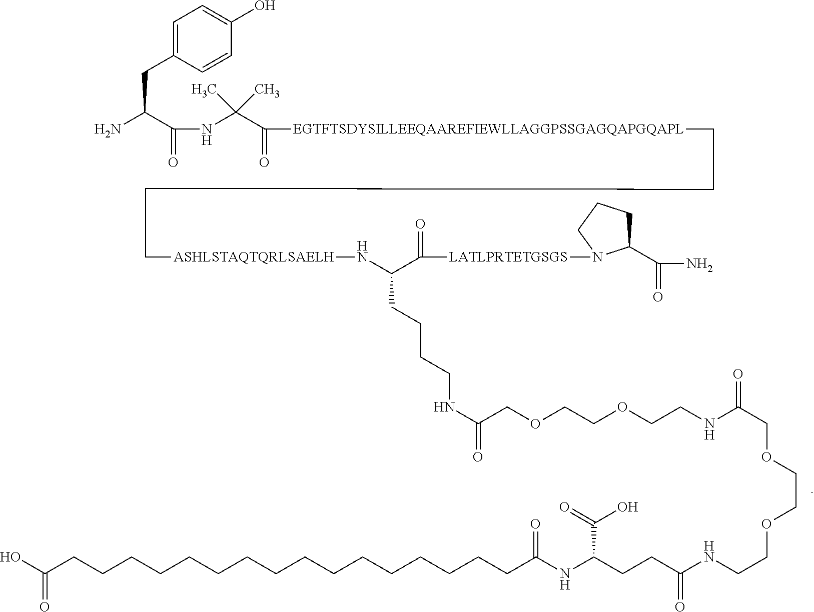

112. A GLP-1-/GIP-/amylin-receptor tri-agonist, which is compound 68

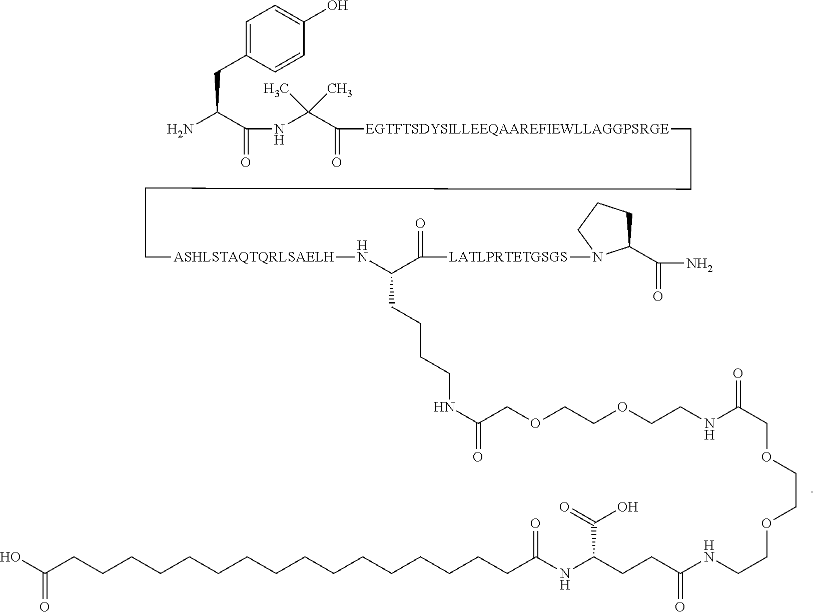

113. A GLP-1-/GIP-/amylin-receptor tri-agonist, which is Compound 77

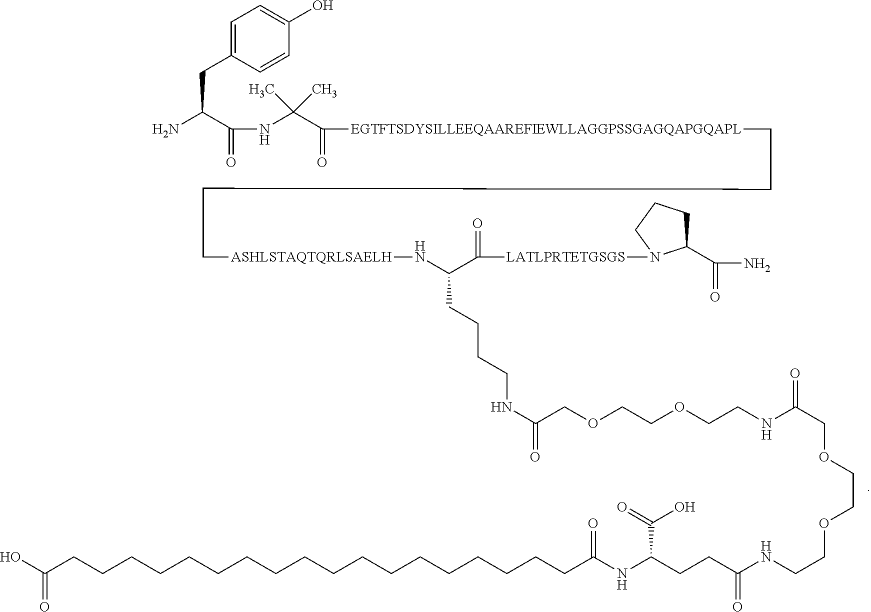

114. The GLP-1-/GIP-/amylin-receptor tri-agonist according to any one of the preceding embodiments wherein the peptide has the amide modification of the C-terminus.
115. The GLP-1-/GIP-/amylin-receptor tri-agonist according to any one of the preceding embodiments which is capable of activating the human GIP receptor.
116. The GLP-1-/GIP-/amylin-receptor tri-agonist according to any one of the preceding embodiments which is capable of activating the human GIP receptor in an assay with whole cells expressing the human GIP receptor.
117. The GLP-1-/GIP-/amylin-receptor tri-agonist according to any one of the preceding embodiments which activates the human GIP receptor in vitro, preferably with an EC₅₀ of less than 125 pM, even more preferably with an EC₅₀ of less than 100 pM, and most preferably with an EC₅₀ of less than 50 pM, when measured without HSA in an assay as described in Example 4.
118. The GLP-1-/GIP-/amylin-receptor tri-agonist according to any one of the preceding embodiments which is capable of activating the human GLP-1 receptor.
119. The GLP-1-/GIP-/amylin-receptor tri-agonist according to any one of the preceding embodiments which is capable of activating the human GLP-1 receptor in an assay with whole cells expressing the human GLP-1 receptor.
120. The GLP-1-/GIP-/amylin-receptor tri-agonist according to any one of the preceding embodiments which activates the human GLP-1 receptor in vitro, preferably with an EC₅₀ of less than 125 pM, even more preferably with an EC₅₀ of less than 100 pM, and most preferably with an EC₅₀ of less than 50 pM, when measured without HSA in an assay as described in Example 4.
121. The GLP-1-/GIP-/amylin-receptor tri-agonist according to any one of the preceding embodiments which is capable of activating the human amylin receptor.
122. The GLP-1-/GIP-/amylin-receptor tri-agonist according to any one of the preceding embodiments which is capable of activating the human amylin receptor in an assay with whole cells expressing the human amylin receptor.
123. The GLP-1-/GIP-/amylin-receptor tri-agonist according to any one of the preceding embodiments which activates the human amylin receptor in vitro, preferably with an EC₅₀ of less than 125 pM, even more preferably with an EC₅₀ of less than 100 pM, and most preferably with an EC₅₀ of less than 50 pM, when measured without HSA in an assay as described in Example 4.
124. The GLP-1-/GIP-/amylin-receptor tri-agonist according to any one of the preceding embodiments which is capable of activating the human GIP, GLP-1, and amylin receptors.
125. The GLP-1-/GIP-/amylin-receptor tri-agonist according to any one of the preceding embodiments which is capable of activating the human GIP, GLP-1, and amylin receptors in assays with whole cells expressing the human GIP receptor, GLP-1, and amylin receptors.
126. The GLP-1-/GIP-/amylin-receptor tri-agonist according to any one of the preceding embodiments which activates the human GIP, GLP-1 and amylin receptors in vitro, preferably with an EC₅₀ of less than 125 pM, even more preferably with an EC₅₀ of less than 100 pM, and most preferably with an EC₅₀ of less than 50 pM, when measured without HSA in assays as described in Example 4.
127. The GLP-1-/GIP-/amylin-receptor tri-agonist according to any one of the preceding embodiments, which activates the human GIP, GLP-1 and amylin receptors in vitro, when measured without HSA in assays as described in Example 4, and which has a potency ratio of less than 50.
128. The GLP-1-/GIP-/amylin-receptor tri-agonist according to embodiment 127, wherein the potency ratio is less than 20.
129. The GLP-1-/GIP-/amylin-receptor tri-agonist according to embodiment 127 or 128, wherein the potency ratio is less than 15, and most preferred less than 11.
130. The GLP-1-/GIP-/amylin-receptor tri-agonist according to any one of the embodiments 81 to 114 which has improved pharmacokinetic properties.
131. The GLP-1-/GIP-/amylin-receptor tri-agonist according to any one of the embodiments 81 to 114 which has an increased half-life.
132. The GLP-1-/GIP-/amylin-receptor tri-agonist according to embodiment 131, which has an increased half-life of 40 hours to 145 hours, when determined in minipigs, preferably of 90 hours to 140 hours, even more preferably 85 hours to 125 hours.
133. The GLP-1-/GIP-/amylin-receptor tri-agonist according to any one of the embodiments 81 to 132, which has improved chemical stability.
134. The GLP-1-/GIP-/amylin-receptor tri-agonist according to embodiment 133, which has improved chemical stability and which has a purity loss of no more than 6.0 percent per week, such as determined in Example 7 described herein, preferably a purity loss of less than 3.0 percent per week, such as determined in Example 7 described herein.
135. The GLP-1-/GIP-/amylin-receptor tri-agonist according to any one of the embodiments 81 to 134 which has the effect in vivo of reducing food intake in normal weight rats, as determined in the experimental protocol for efficacy testing on appetite, such as Example 5 described herein; or which has the effect in vivo of reducing food intake in DIO rats, as determined in sub-chronic treatment in DIO rats, such as Example 8 described herein.
136. A pharmaceutically acceptable salt of the GLP-1-/GIP-/amylin-receptor triple agonist according to any one of the preceding embodiments.
137. A pharmaceutical composition comprising a GLP-1-/GIP-/amylin-receptor triple agonist according to any one of the preceding embodiments, and one or more pharmaceutically acceptable excipients.
138. The pharmaceutical composition according to embodiment 137, which is for oral or for subcutaneous administration.
139. The pharmaceutical composition according to the embodiment 137 or embodiment 138, which is a solid pharmaceutical composition.
140. The solid pharmaceutical composition according to embodiment 139, which is a tablet.
141. The solid pharmaceutical composition according to embodiment 139 or embodiment 140, comprising a salt of N-[8-(2-hydroxybenzoyl)amino]caprylate, preferably sodium N-(8-(2-hydroxybenzoyl)amino)caprylate and magnesium stearate.
142. The solid pharmaceutical composition according to any one of the embodiments 139 to 141, comprising 75-600 mg sodium N-(8-(2-hydroxybenzoyl)amino)caprylate and 7-8.5 mg magnesium stearate.
143. The pharmaceutical composition according to any one of embodiments 137 to 142, which is for dosing approximately once daily, such as once every 12-36 hours, such as once every 18-30 hours, such as approximately once every 24 hours; or which is for dosing approximately once weekly, such as once every 6-8 days.
144. An injection device comprising the GLP-1-/GIP-/amylin-receptor tri-agonist according to any one of embodiments 1 to 136, or the pharmaceutical composition according to any one of embodiments 137 to 143.
145. The GLP-1-/GIP-/amylin-receptor tri-agonist peptide according to any of embodiments 1 to 78 for use as an intermediate in the manufacture of a GLP-1-/GIP-/amylin-receptor tri-agonist peptide derivative according to any of embodiments 81 to 135.
146. The GLP-1-/GIP-/amylin-receptor triple agonist according to any one of embodiments 1 to 136 or the pharmaceutical composition according to any one of embodiments 137 to 143 for use as a medicament.
147. The GLP-1-/GIP-/amylin-receptor triple agonist according to any one of embodiments 1 to 136 or the pharmaceutical composition according to any one of embodiments 137 to 143 for use in the treatment of type 2 diabetes, obesity, metabolic dysfunction-associated steatohepatitis (MASH), and/or cardiovascular disease.
148. The GLP-1-/GIP-/amylin-receptor triple agonist according to any one of embodiments 1 to 136 or the pharmaceutical composition according to any one of embodiments 137 to 143, for use in the treatment of a subject with an initial body mass index (BMI) of 25 or more, 27 or more, or 28 or more, or 30 or more.
149. The GLP-1-/GIP-/amylin-receptor triple agonist according to any one of embodiments 1 to 136 or the pharmaceutical composition according to any one of embodiments 137 to 143, for use in the treatment of a subject with an initial body mass index (BMI) of 25 or more, 27 or more, or 28 or more, 30 or more; and in the presence of at least one weight-related co-morbidity.
150. The GLP-1-/GIP-/amylin-receptor triple agonist according to any one of embodiments 1 to 136 or the pharmaceutical composition according to any one of embodiments 137 to 143, for use as an adjunct to a reduced-calorie diet and increased physical activity for chronic weight management in an adult subject suffering from obesity, i.e. with an initial body mass index (BMI) of 25 or more, or 27 or more, or 28 or more, 30 or more; or in an adult subject suffering from overweight, i.e. with an initial body mass index (BMI) of 23 or more, or 24 or more, or 25 or more, or 27 or more.
151. The GLP-1-/GIP-/amylin-receptor triple agonist according to any one of embodiments 1 to 136 or the pharmaceutical composition according to any one of embodiments 137 to 143, for use as an adjunct to a reduced-calorie diet and increased physical activity for chronic weight management in an adult subject suffering from obesity, i.e. with an initial body mass index (BMI) of 25 or more, or 27 or more, or 28 or more, 30 or more; or in an adult subject suffering from overweight, i.e. with an initial body mass index (BMI) of 23 or more, or 24 or more, or 25 or more, or 27 or more; and in the presence of at least one weight-related co-morbidity.
152. The use according to embodiment 149 or embodiment 151, wherein the at least one weight-related co-morbidity is selected from the group consisting of hypertension, dysglycaemia (prediabetes or type 2 diabetes), dyslipidaemia, high cholesterol, cardiovascular disease and obstructive sleep apnoea.
153. Use of a GLP-1-/GIP-/amylin-receptor triple agonist according to any one of embodiments 1 to 136 in the manufacture of a medicament for the treatment of type 2 diabetes, obesity, metabolic dysfunction-associated steatohepatitis (MASH), and/or cardiovascular disease.
154. Use of a GLP-1-/GIP-/amylin-receptor triple agonist according to any one of embodiments 1 to 136 in the manufacture of a medicament for the treatment of a subject with an initial body mass index (BMI) of 25 or more, 27 or more, or 28 or more, 30 or more; and optionally in the presence of at least one weight-related co-morbidity.
155. Use of a GLP-1-/GIP-/amylin-receptor triple agonist according to any one of embodiments 1 to 136 in the manufacture of a medicament for the treatment of an adult subject suffering from obesity, i.e. with an initial body mass index (BMI) of 25 or more, or 27 or more, or 28 or more, 30 or more; or an adult subject suffering from overweight, i.e. with an initial body mass index (BMI) of 23 or more, or 24 or more, or 25 or more, or 27 or more; and optionally in the presence of at least one weight-related co-morbidity.
156. Use of a GLP-1-/GIP-/amylin-receptor triple agonist according to any one of embodiments 1 to 136 in the manufacture of a medicament for chronic weight management in an adult subject suffering from obesity, i.e. with an initial body mass index (BMI) of 25 or more, or 27 or more, or 28 or more, 30 or more; or in an adult subject suffering from overweight, i.e. with an initial body mass index (BMI) of 23 or more, or 24 or more, or 25 or more, or 27 or more; and optionally in the presence of at least one weight-related co-morbidity.
157. The use according to embodiment 155 or embodiment 155, wherein the medicament is an adjunct to a reduced-calorie diet and increased physical activity for chronic weight management in an adult subject suffering from obesity or overweight.
158. A method for treating type 2 diabetes, obesity, metabolic dysfunction-associated steatohepatitis (MASH), and/or cardiovascular disease comprising administering a pharmaceutically relevant amount of a GLP-1-/GIP-/amylin-receptor triple agonist according to any one of embodiments 1 to 136 to a subject in need thereof.
159. A method of treating a human subject with an initial body mass index (BMI) of 25 or more, 27 or more, or 28 or more, or 30 or more, comprising administering to said human subject a pharmaceutically relevant amount of a GLP-1-/GIP-/amylin-receptor triple agonist according to any one of embodiments 1 to 136.
160. A method for reducing excess body weight in a human subject, in combination with a reduced-calorie diet and increased physical activity, comprising administering to said human subject a pharmaceutically relevant amount of a GLP-1-/GIP-/amylin-receptor triple agonist according to any one of embodiments 1 to 136.
161. The method according to embodiment 159 or embodiment 160, wherein said human subject is an adult subject, suffering from overweight and has an initial body mass index (BMI) of 23 or more, or 24 or more, or 25 or more, or 27 or more.
162. The method according to embodiment 159 or embodiment 160, wherein said human subject is an adult subject, suffering from obesity and has an initial body mass index (BMI) of 25 or more, or 27 or more, or 28 or more, 30 or more.
163. The method according to any one of embodiments 159 to 162, wherein said human subject has at least one weight-related comorbidity selected from the group consisting of hypertension, dysglycaemia (prediabetes or type 2 diabetes), dyslipidaemia, high cholesterol, cardiovascular disease and obstructive sleep apnoea.
164. A method for preparing the GLP-1-/GIP-/amylin-receptor triple agonist according to any one of embodiments 1 to 136.
165. The method according to embodiment 164, comprises a step of solid phase peptide synthesis.

### EXAMPLES

### Materials and Methods

### List of Abbreviations

The following abbreviations are used in the following, in alphabetical order:
Ado: 8-amino-3,6-dioxaoctanoic acid
Aib: 2-aminoisobutyric acid
amu: atomic mass unit
BHK Baby Hamster Kidney
Boc: t-butyloxycarbonyl
CAD: Charged Aerosol Detector
cAMP: cyclic adenosine monophosphate
CRE: cAMP response element
DCM: dichloromethane
DIC: N,N′-diisopropylcarbodiimide
DIO: Diet Induced Obese
DMB: 2,4-dimethoxybenzyl
DMEM: Dulbecco's Modified Eagle's Medium
DMF: N,N-dimethyl formamide
DTT: 1,4-dithiothreitol
EC50: half maximal effective concentration
EDTA: ethylenediaminetetraacetic acid
ES: Electrospray
FBS: Foetal Bovine Serum
Fmoc: 9-fluorenylmethyloxycarbonyl
FWHM: Full Width at Half Maximum
GIP: Glucose-dependent Insulinotropic Polypeptide
GLP-1: Glucagon-Like Peptide-1
hAMYR3: human Amylin receptor 3
hGIPR: human Glucose-dependent Insulinotropic Polypeptide Receptor
hGLP-1R: human Glucagon-Like Peptide-1 Receptor
HEPES: N-(2-Hydroxyethyl)piperazine-N-(2-ethanesulfonic acid)
HFIP: 1,1,1,3,3,3-hexafluoro-2-propanol or hexafluoroisopropanol
HPLC: High Performance Liquid Chromatography
HSA: Human Serum Albumin
i.v.: intravenously
LCMS or LC-MS: Liquid Chromatography Mass Spectrometry
LLoQ: Lower limit of Quantitation
Luc: luciferase
MeCN: acetonitrile
MRI: Magnetic resonance imaging
MS: Mass Spectroscopy
Mtt: 4-methyltrityl
NCA: non-compartmental pharmacokinetic method
nd: not determined
OtBu: tert-butoxy
Oxyma Pure®: cyano-hydroxyimino-acetic acid ethyl ester
Pbf: 2,2,4,6,7-pentamethyldihydrobenzofuran-5-sulfonyl
PBS: phosphate-buffered saline
PK: pharmacokinetic
QD: quaque die (once a day)
QTof: Quadrupole Time of Flight
RAMP3: receptor modifying protein 3
RT: Room Temperature
s.c.: subcutaneously
SD: Sprague Dawley
SEM: Standard Error of Mean
SPPS: Solid Phase Peptide Synthesis
tBu: tert-butyl
TFA: trifluoroacetic acid
TIPS: triisopropylsilane
TQ: Triple Quadrupole
Trt: triphenylmethyl or trityl
UPLC: Ultra Performance Liquid Chromatography
UV: Ultraviolet

### Fatty Diacid and Special Amino Acid Building Blocks

For synthesis of octadecanedioic acid mono-tert-butyl ester (C₁₈ diacid mono-tert-butyl ester): see patent application WO 2010/102886 (pages 27-28). The corresponding mono-tert-butyl esters of C₁₂-C₂₀ diacid, in particular C₁₆ diacid and C₂₀ diacid, can be prepared accordingly.

Fmoc-Leu-Ser(ψ^{Me},^{Me}pro)-OH, Fmoc-Tyr(tBu)-Ser(ψ^{Me},^{Me}pro)-OH, and Fmoc-Gly-(DMB)Gly-OH were commercially available from TechnoComm Ltd.

### General Method for Peptide Synthesis

The preparation of the peptides (for reference compounds and compounds of the invention) was carried out with SPPS using Fmoc based chemistry on a Symphony X from Protein Technologies, a PurePep Chorus from Protein Technologies, a MultiPep 2 from CEM, a Vapourtec RS-500 from Vapourtec or a CS136XT from CSBioon. The Fmoc-protected amino acids used in the methods were the standard recommended: Fmoc-Ala-OH, Fmoc-Arg(Pbf)-OH, Fmoc-Asp(OtBu)-OH, Fmoc-Asn(Trt)-OH, Fmoc-Cys(Trt)-OH, Fmoc-Gln(Trt)-OH, Fmoc-Glu(OtBu)-OH, Fmoc-Gly-OH, Fmoc-His(Trt)-OH, Fmoc-Ile-OH, Fmoc-Leu-OH, Fmoc-Lys(Boc)-OH, Fmoc-Phe-OH, Fmoc-Pro-OH, Fmoc-Ser(tBu)-OH, Fmoc-Thr(tBu)-OH, Fmoc-Trp(Boc)-OH, Fmoc-Tyr(tBu)-OH, Fmoc-Val-OH, Fmoc-Lys(Mtt)-OH, Boc-His(Trt)-OH, Fmoc-Aib-OH, Fmoc-Glu-OtBu, and Fmoc-Ado-OH supplied from e.g. Gyros Protein Technologies, Bachem, Iris Biotech, or NovabioChem. The building blocks Fmoc-Leu-Ser(ψ^{Me,Me}pro)-OH, Fmoc-Tyr(tBu)-Ser(ψ^{Me,Me}pro)-OH, and Fmoc-Gly-(DMB)Gly-OH, e.g. commercially available from TechnoComm Ltd, were introduced where applicable.

Fmoc-PAL AM resin or Rink-Amide AM resin were used, which were commercially available from NovabioChem. The subsequent amino acids were introduced in a stepwise procedure by the Symphony X peptide synthesizer following the SPPS principles.

Fmoc-deprotection was achieved with 20% piperidine in DMF with 0.1 M Oxyma Pure for 2×10 min. Introduction of the substituent (i.e. protraction moiety comprising a “protractor” P and an optional “linker L_{P}”) at the alpha-position of the N-terminal amino acid was accomplished using a standard Fmoc-protected amino acid. The peptide couplings were performed with DIC and collidine. Amino acid/Oxyma Pure solutions (0.3 M/0.3 M in DMF at a molar excess of 5-10-fold) was added to the resin first. Then, the same molar equivalent of DIC was added (1.5 M in DMF), followed by collidine (1.5 M in DMF). Most commonly, it was mixed for 2 hours. In some cases, the coupling time was increased, additional DIC was added, or the coupling step was repeated. Afterwards, a capping step was performed with 1 M acetic anhydride in DMF and collidine. Introduction of the protraction moiety at the epsilon-nitrogen of a lysine (Lys, K) within the sequence was achieved using Fmoc-Lys(Mtt)-OH. Following the synthesis of the peptide backbone sequence the Mtt group was removed by treatment with HFIP/DCM/TIPS (75:23:2) (5 min), followed by a wash with DCM. The resin was then resuspended in HFIP/DCM/TIPS (75:23:2) (2×25 min), and subsequently washed with DCM and DMF. The protraction moieties were introduced in a stepwise procedure as described above, using suitably protected building blocks for the linker L_{P}, such as the standard Fmoc-protected amino acids such as Fmoc-8-amino-3,6-dioxaoctanoic acid or Fmoc-Glu-OtBu. Introduction of the protractor, fatty acid group, was achieved using the suitable building block, such as but not limited to, octadecanedioic acid mono-tert-butyl-ester.

### General Cleavage Method

The peptides were cleaved from the resin with TFA/TIPS/H₂O/DTT (90:4:3:3) for 2-3 hours. Hereafter, the peptide was drained into cold diethyl ether, and centrifuged. The ether was decanted off, and the peptide precipitate was washed with ether two additional times.

### General Method for Purification and Quantification of the Derivative

The crude peptide was dissolved in acetic acid/MeCN/Milli-Q water (45:10:45 or 40:20:40) and orthogonally purified by reversed-phase preparative HPLC (Waters Delta Prep 4000) on a column comprising C18-silica gel. The first elution was performed with an increasing gradient from 20-50% of MeCN in Milli-Q water comprising 1% ammonium bicarbonate. Relevant fractions were analysed with UPLC. Fractions containing the target peptide were pooled and diluted with Milli-Q water (1:1) prior to a second reversed-phase preparative HPLC. The second elution was performed with an increasing gradient from 20-50% of MeCN in Milli-Q water comprising 0.1% TFA. Relevant fractions were analysed with UPLC. Fractions containing the pure target peptide were pooled. The resulting solution was analysed (UPLC, LCMS) and the peptide derivative was quantified using a CAD specific HPLC detector (Thermo-Fischer Vanquish HPLC-CAD). The product was dispensed into glass vials. The vials were capped with Millipore glass fibre prefilters. Freeze-drying afforded the trifluoroacetate salt of the derivative as a white solid.

### Synthesized Compounds

The compounds were prepared using the methods described above.

### Example 1: Reference Compounds

### Reference Compound 1

(GLP-1-/GIP-/amylin-receptor tri-agonist disclosed in WO 2023/288313, example 1, peptide/compound no. 16)

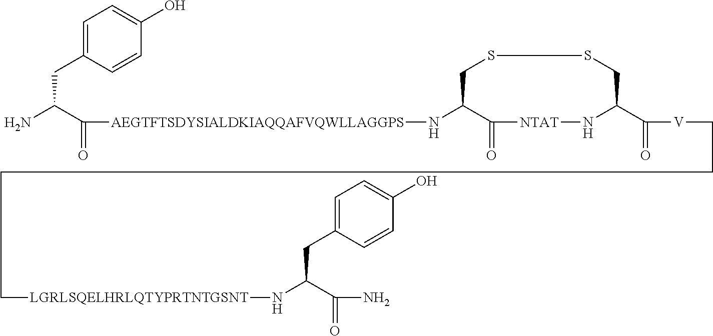

### Reference Compound 2

(Tri-agonist based on conjugation of tirzepatide and cagrilintide)

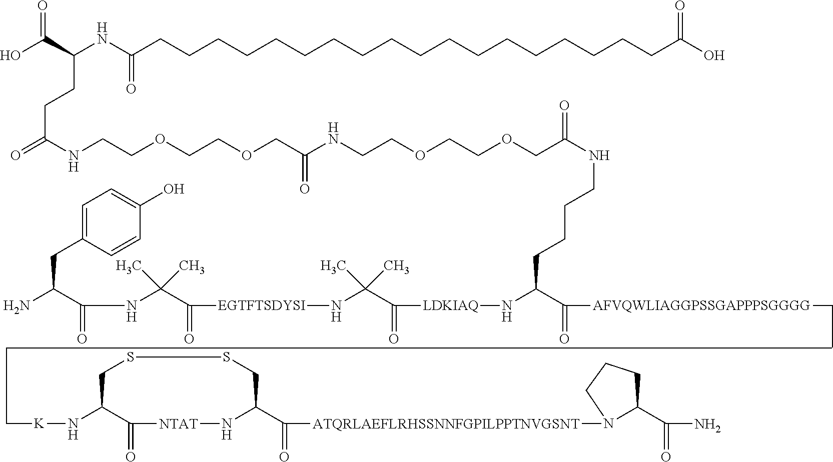

### Reference Compound 3

(Tri-agonist based on conjugation of tirzepatide and amylin receptor agonist disclosed in example 21 in WO 2016/034604)

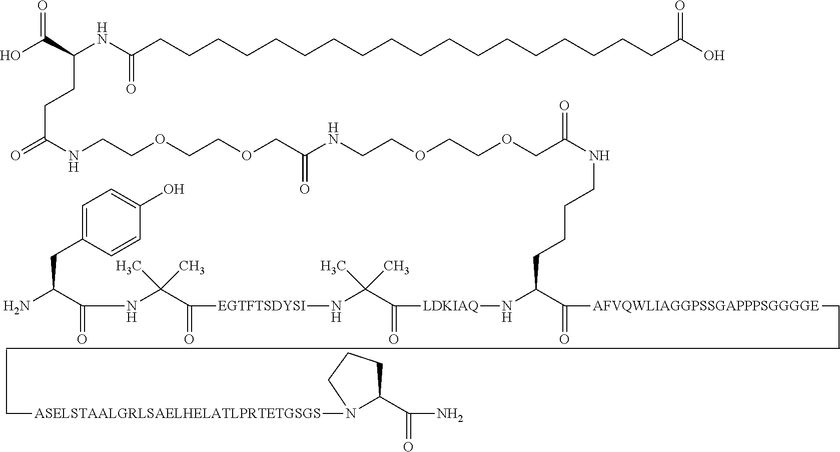

### Reference Compound 4

(GIP receptor agonist disclosed in WO 2019/211451, example 1, compound 31)

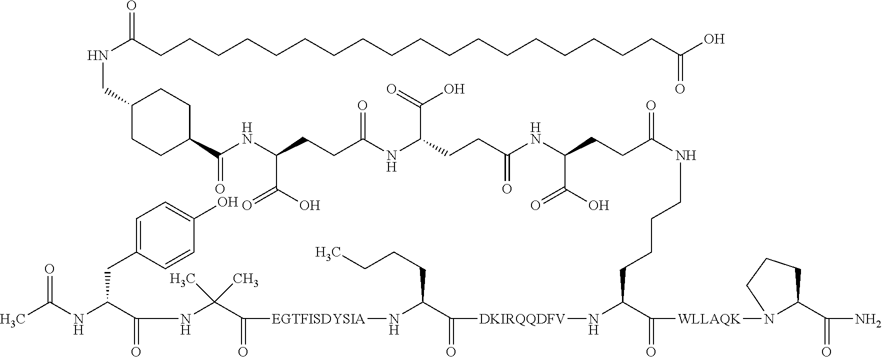

### Reference Compound 5

(GLP-1/GIP co-agonist tirzepatide)

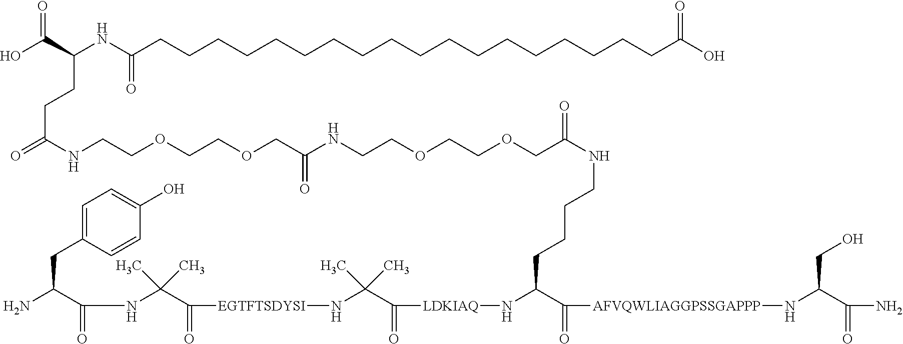

### Reference Compound 6

(Amylin receptor agonist cagrilintide, WO 2012/168432, example 53)

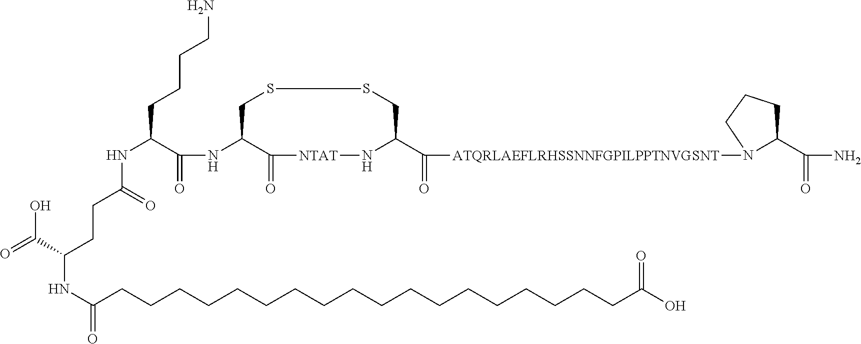

### Reference Compound 7

(GLP-1 receptor agonist semaglutide, WO 2006/097537, example 4)

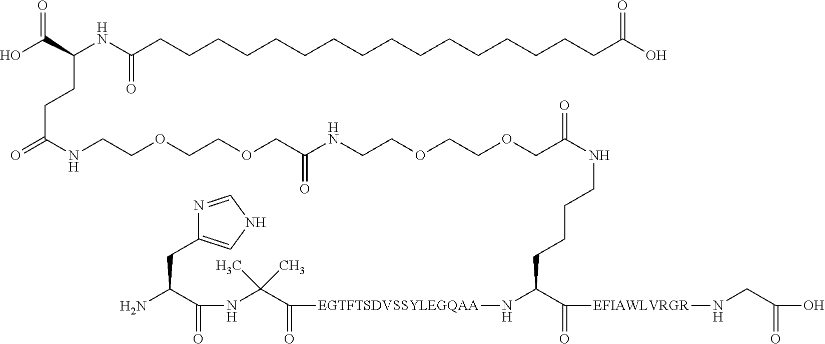

### Example 2: GLP-1-/GIP-/Amylin-Receptor Tri-Agonists According to the Invention

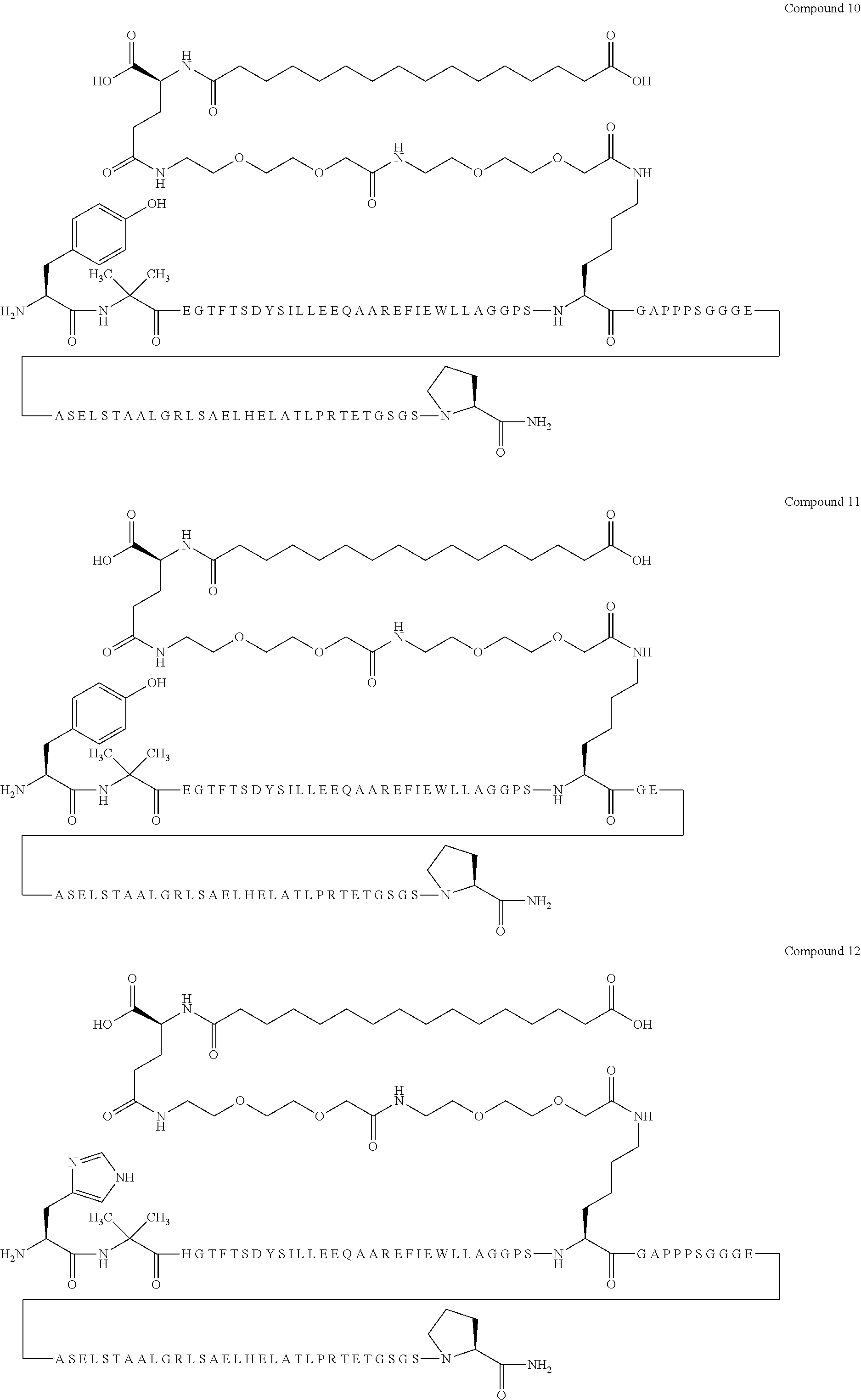

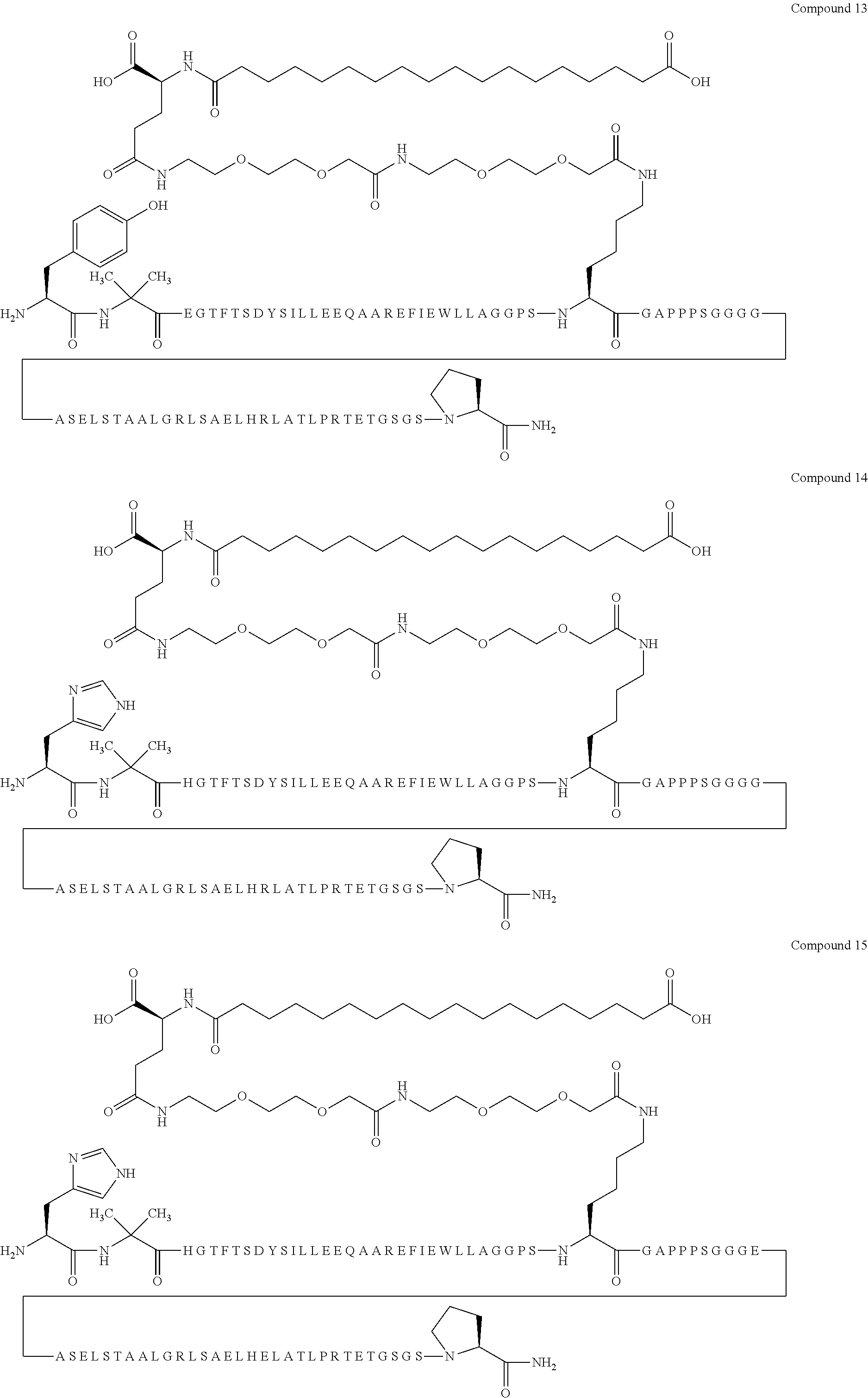

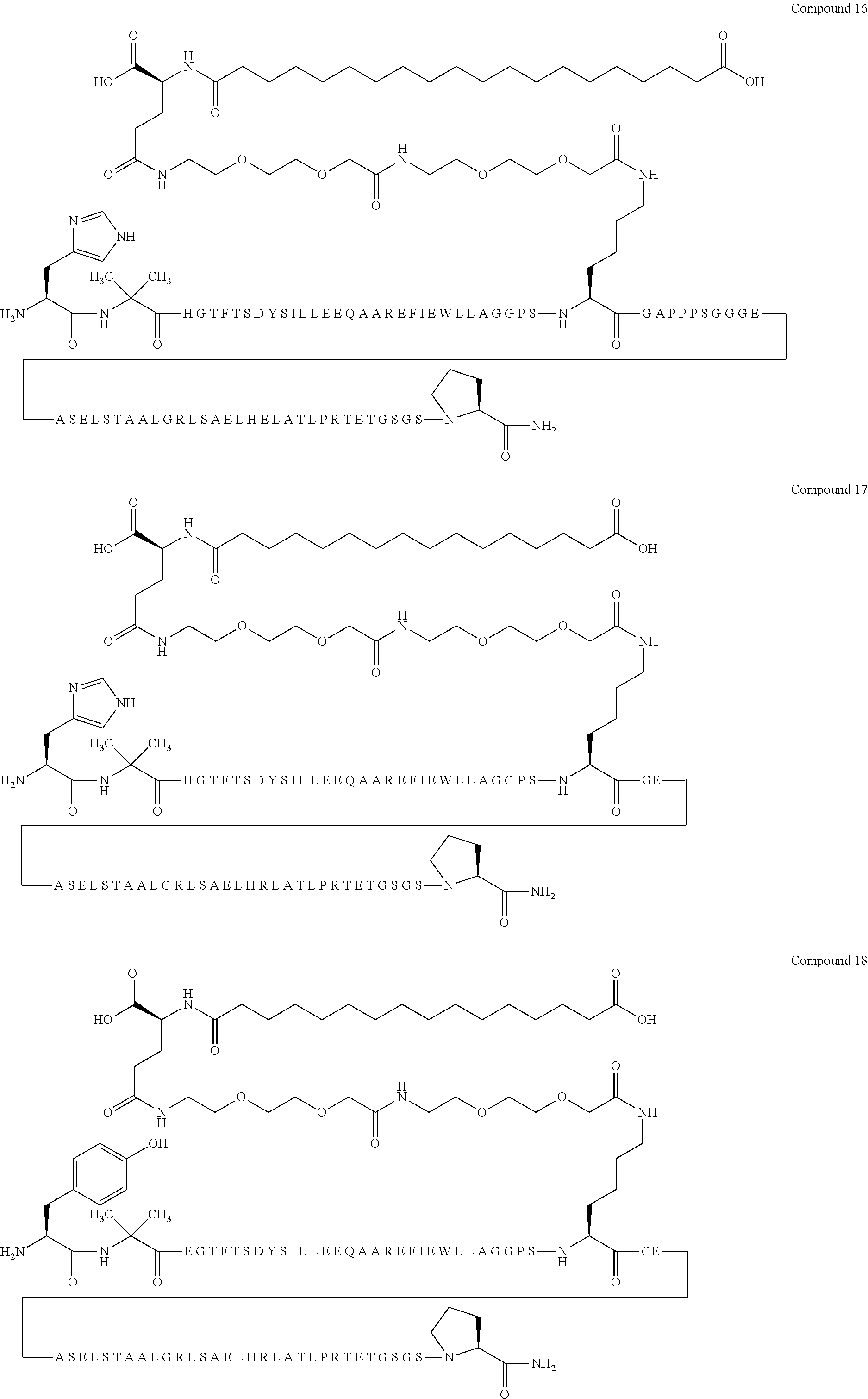

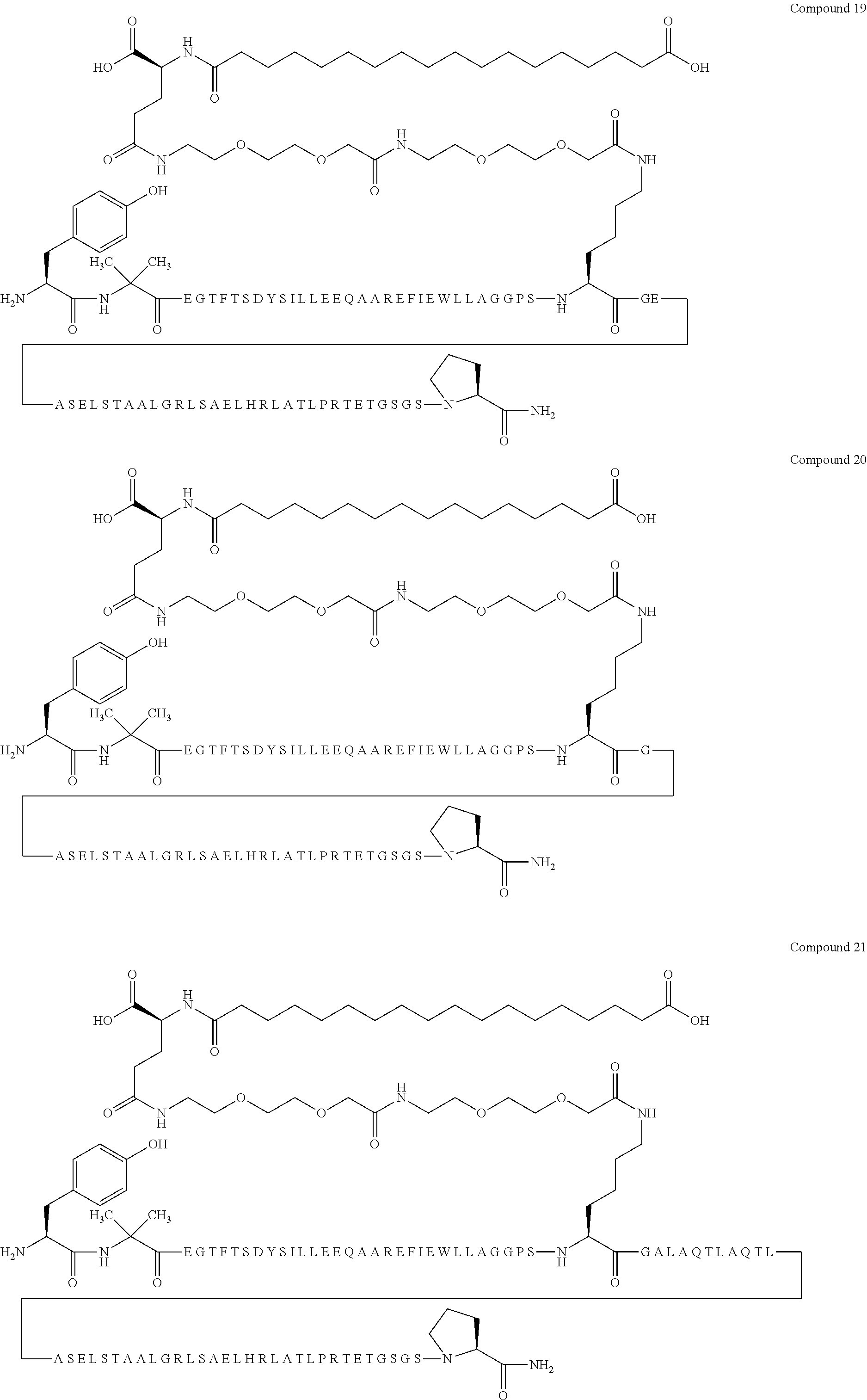

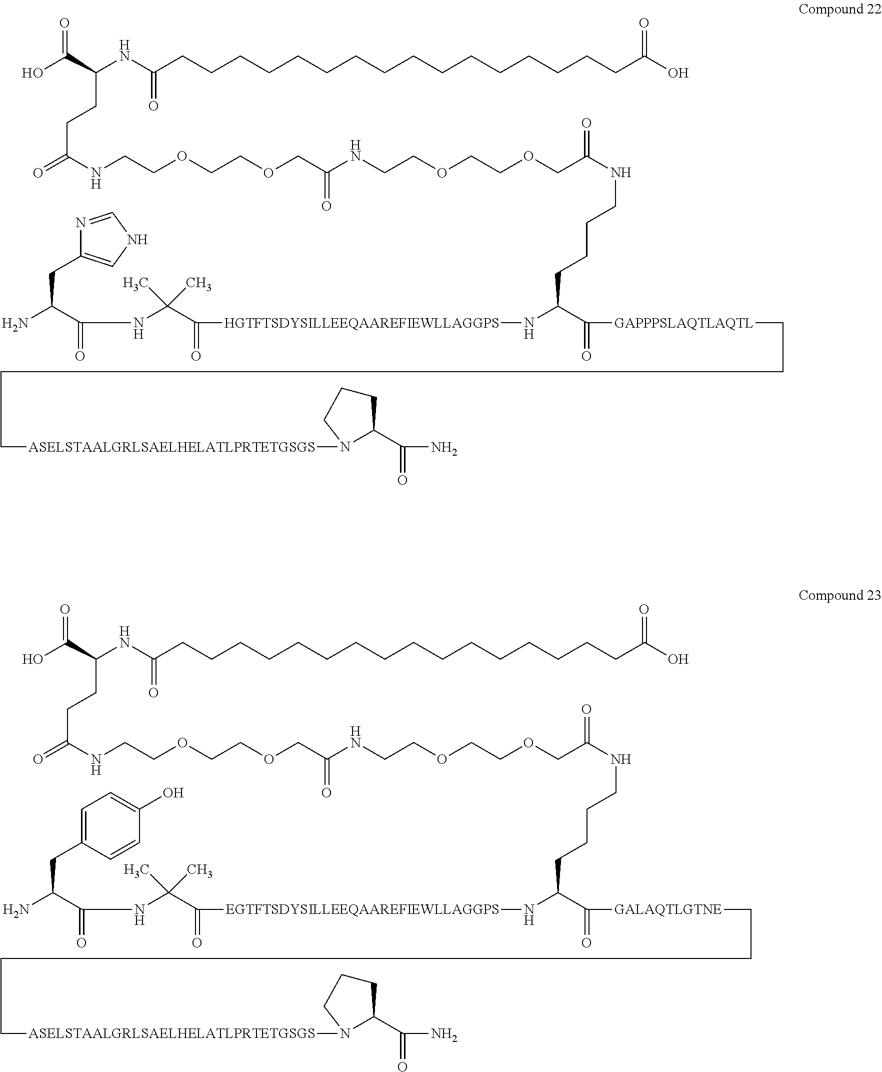

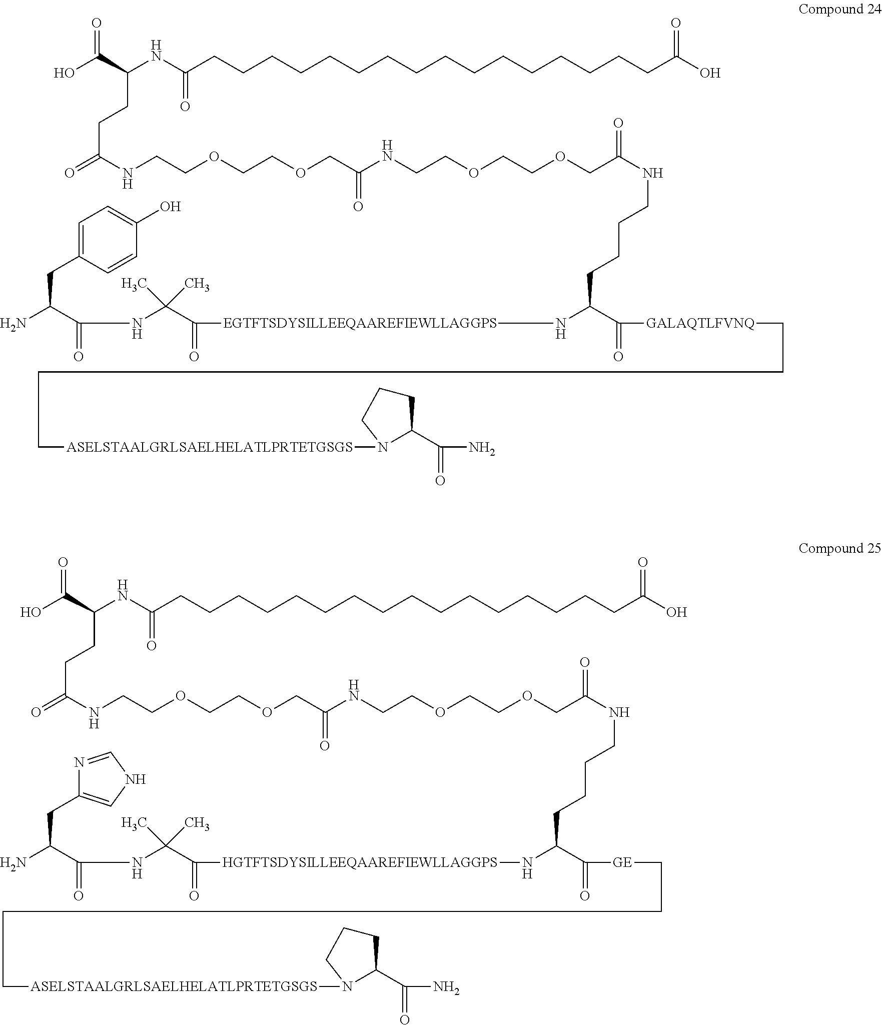

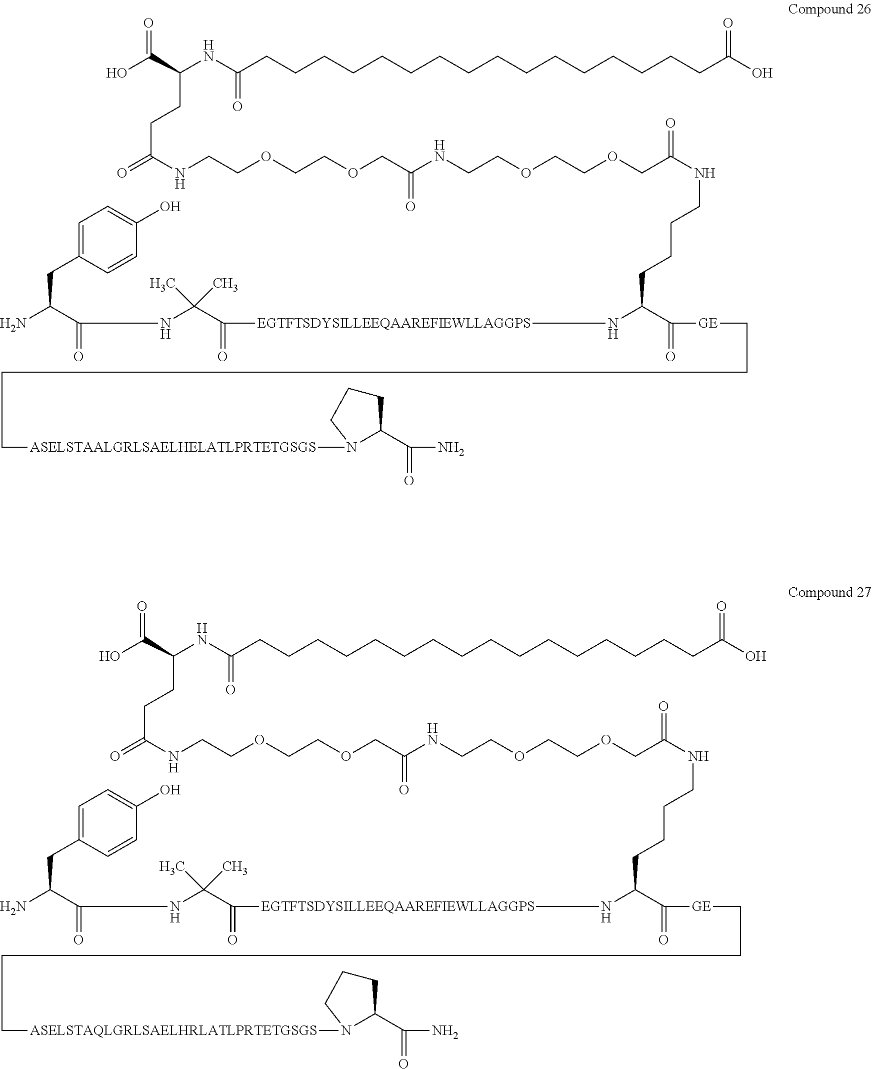

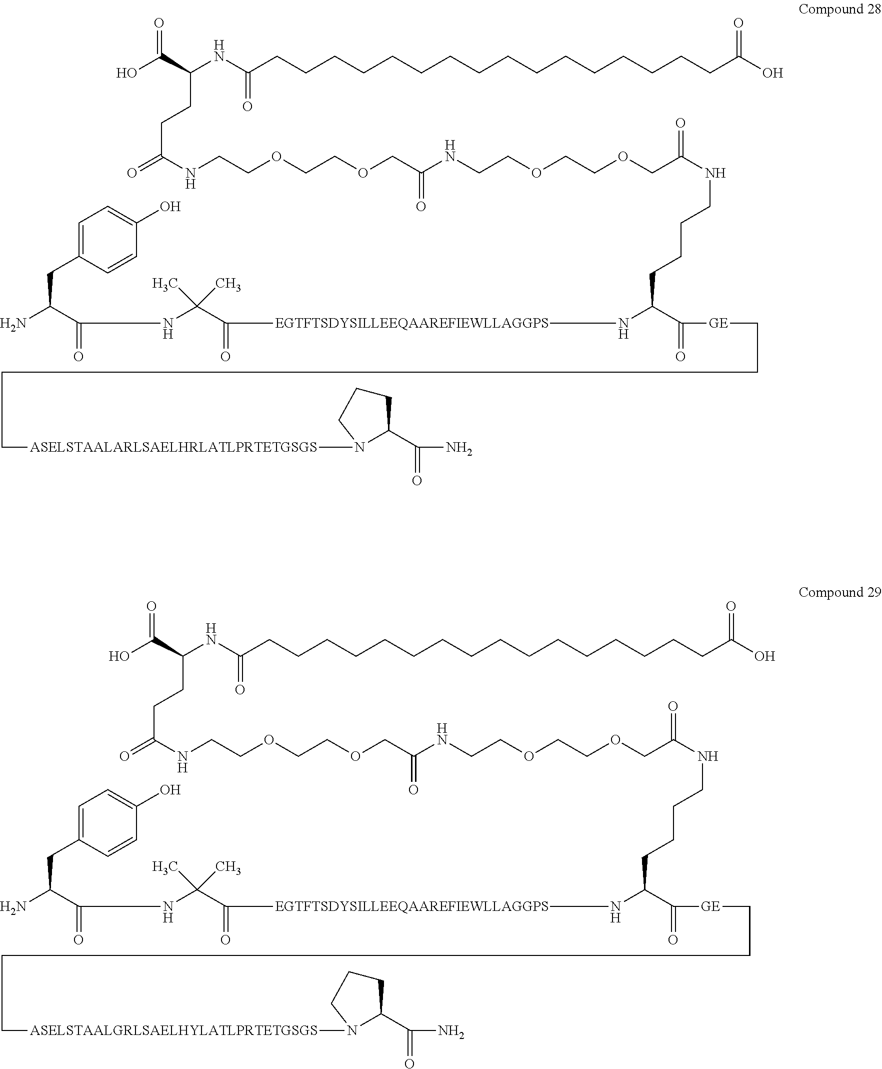

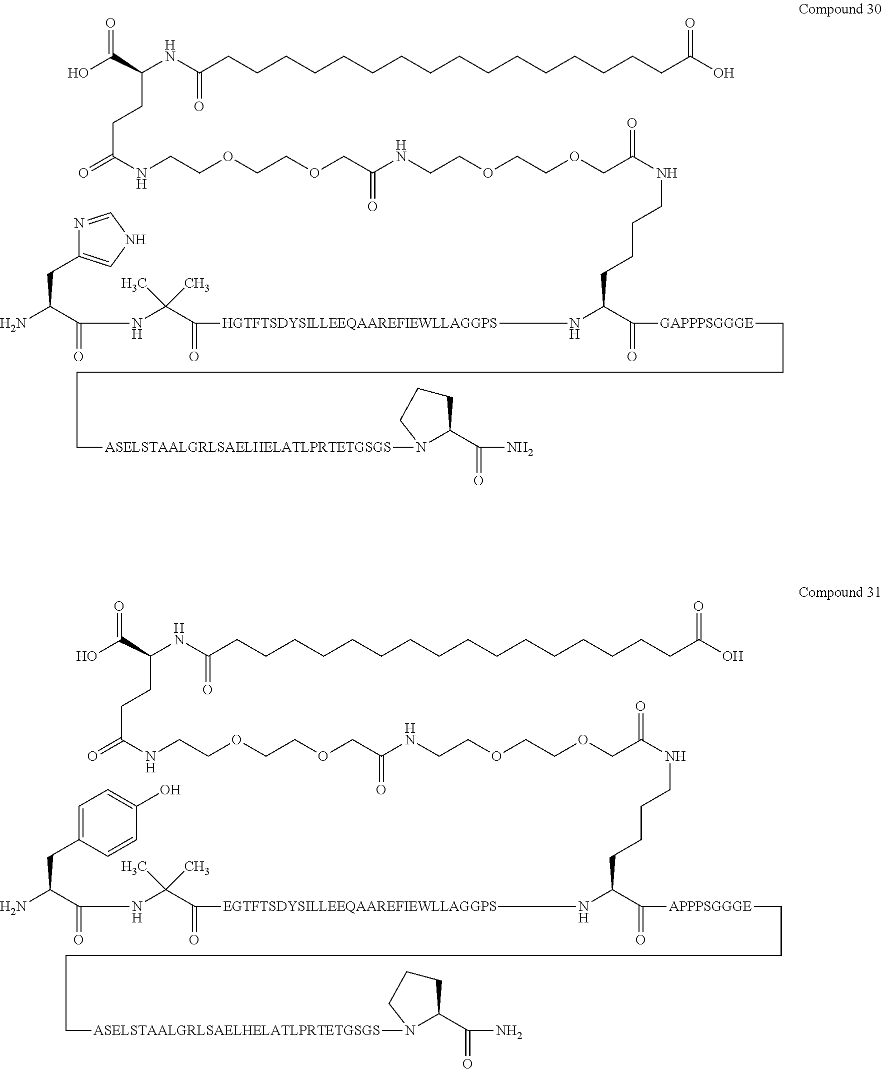

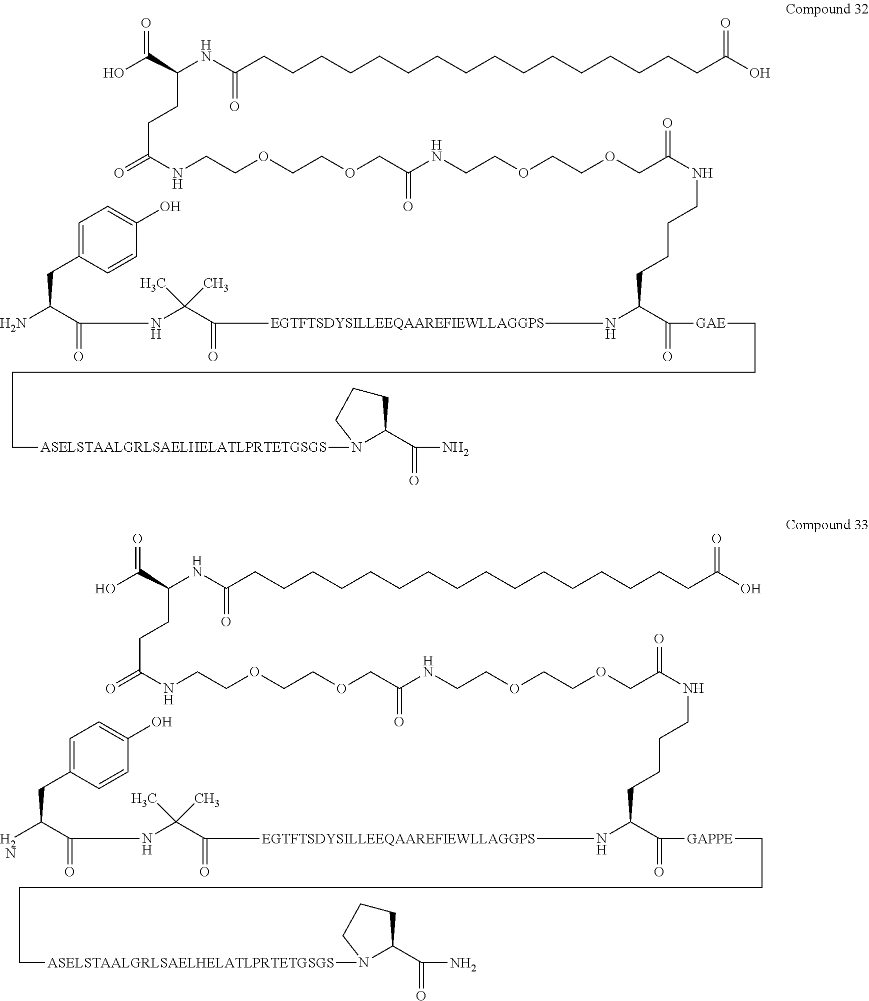

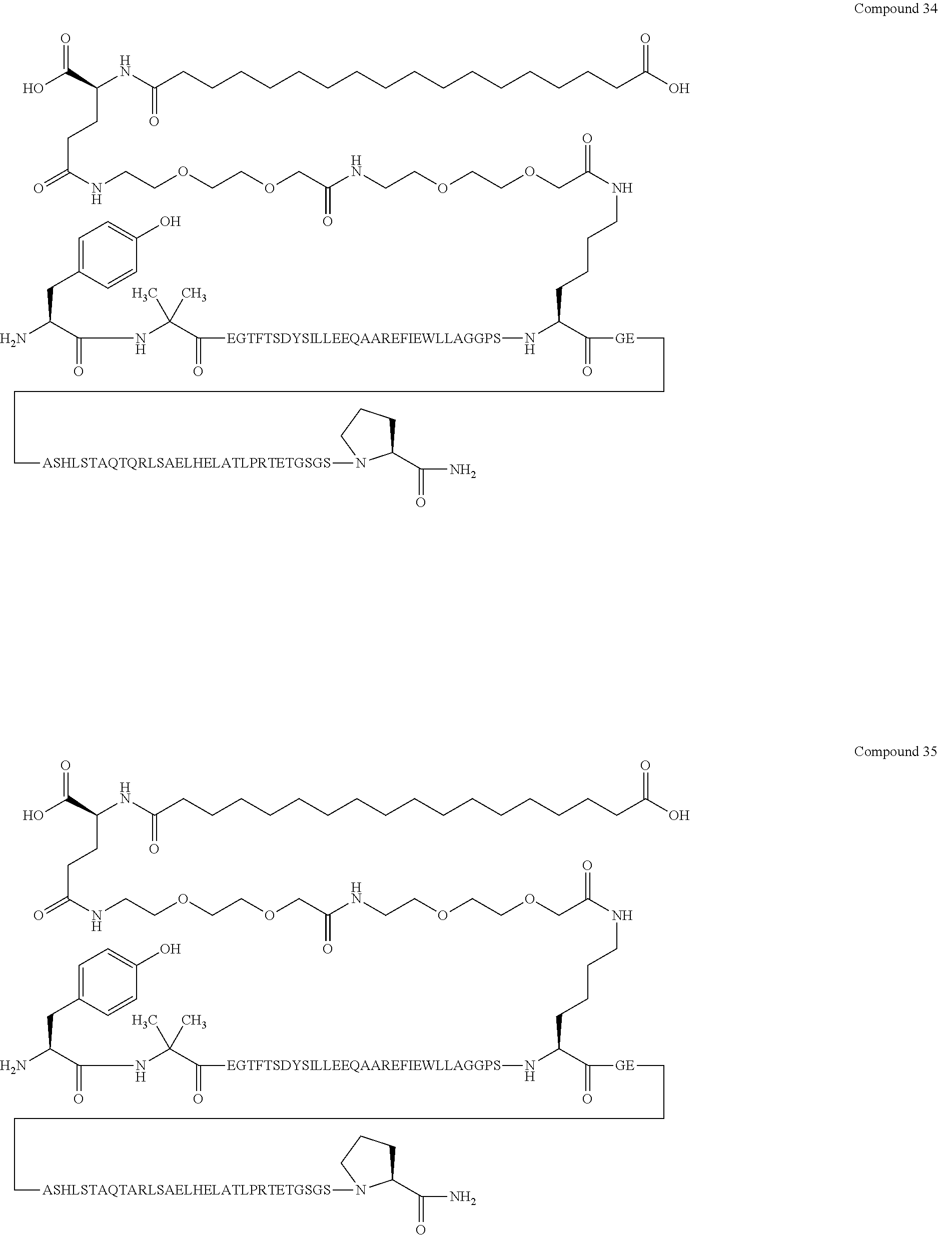

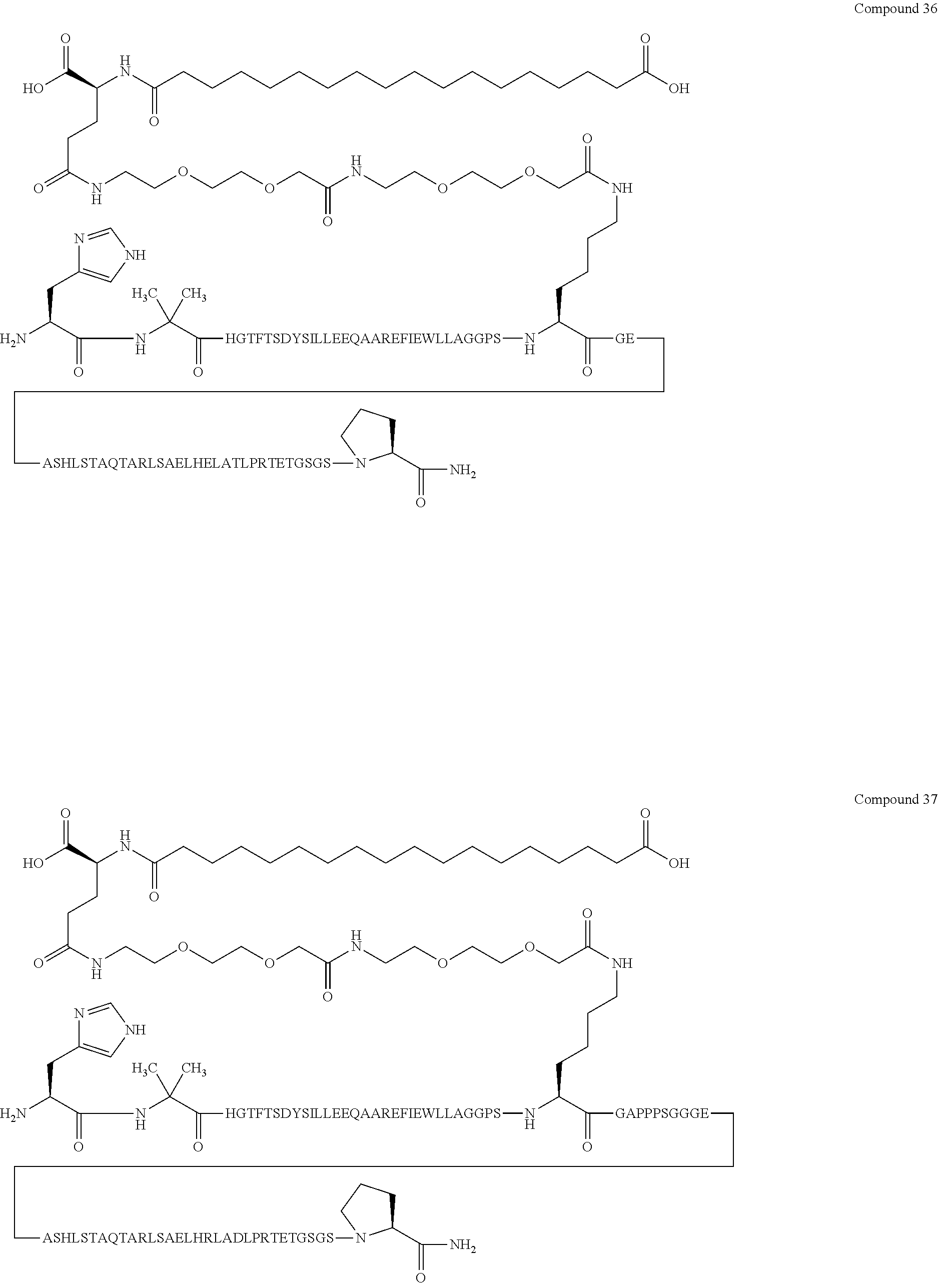

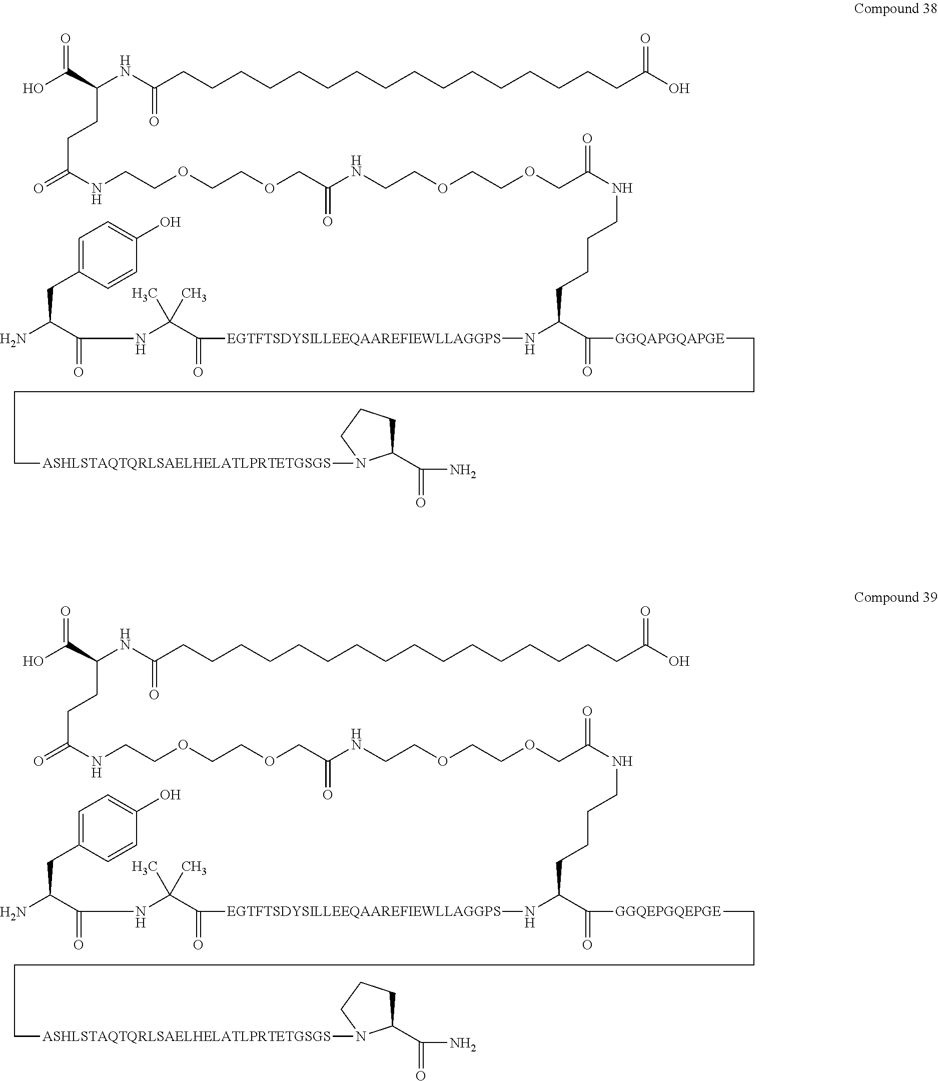

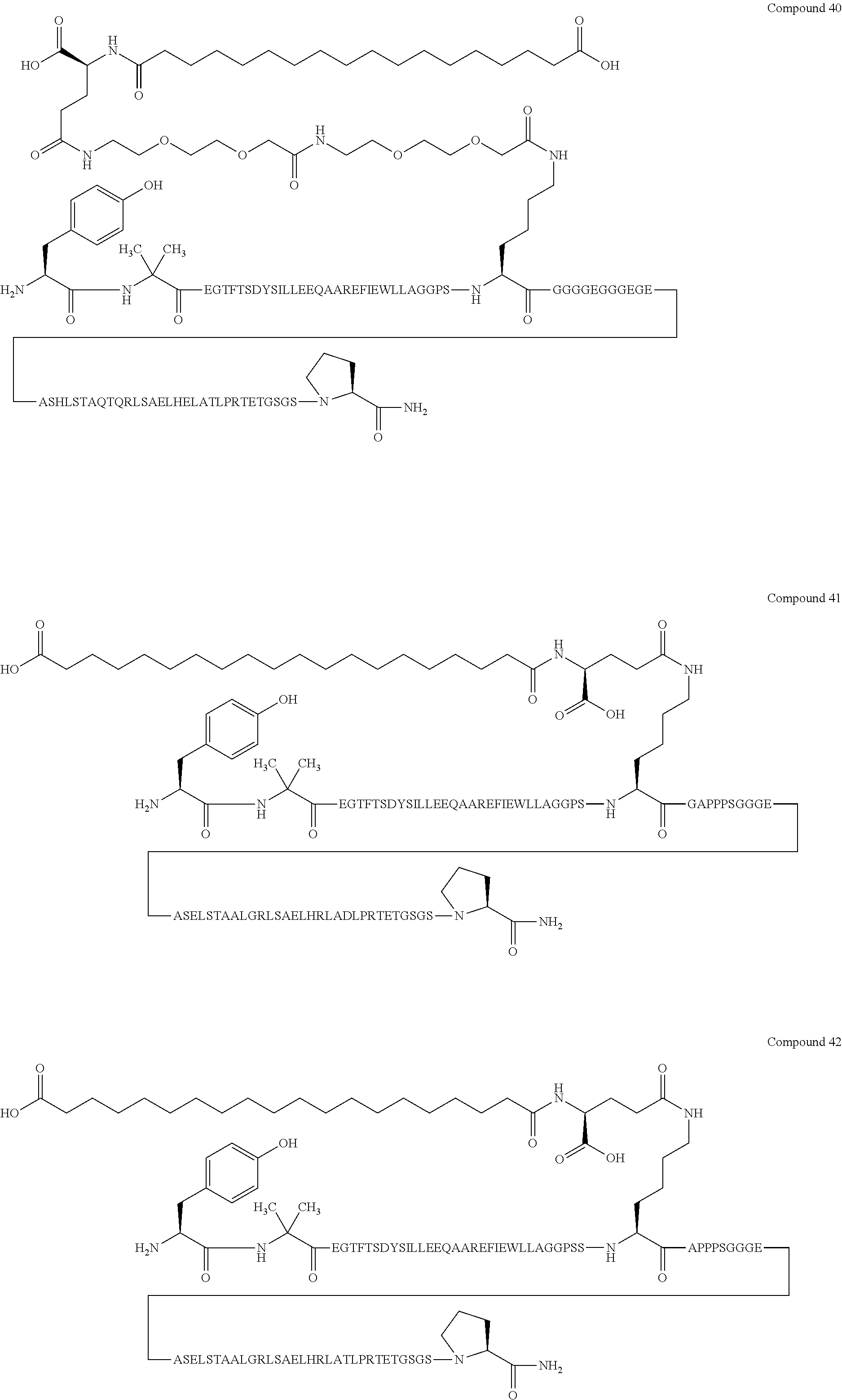

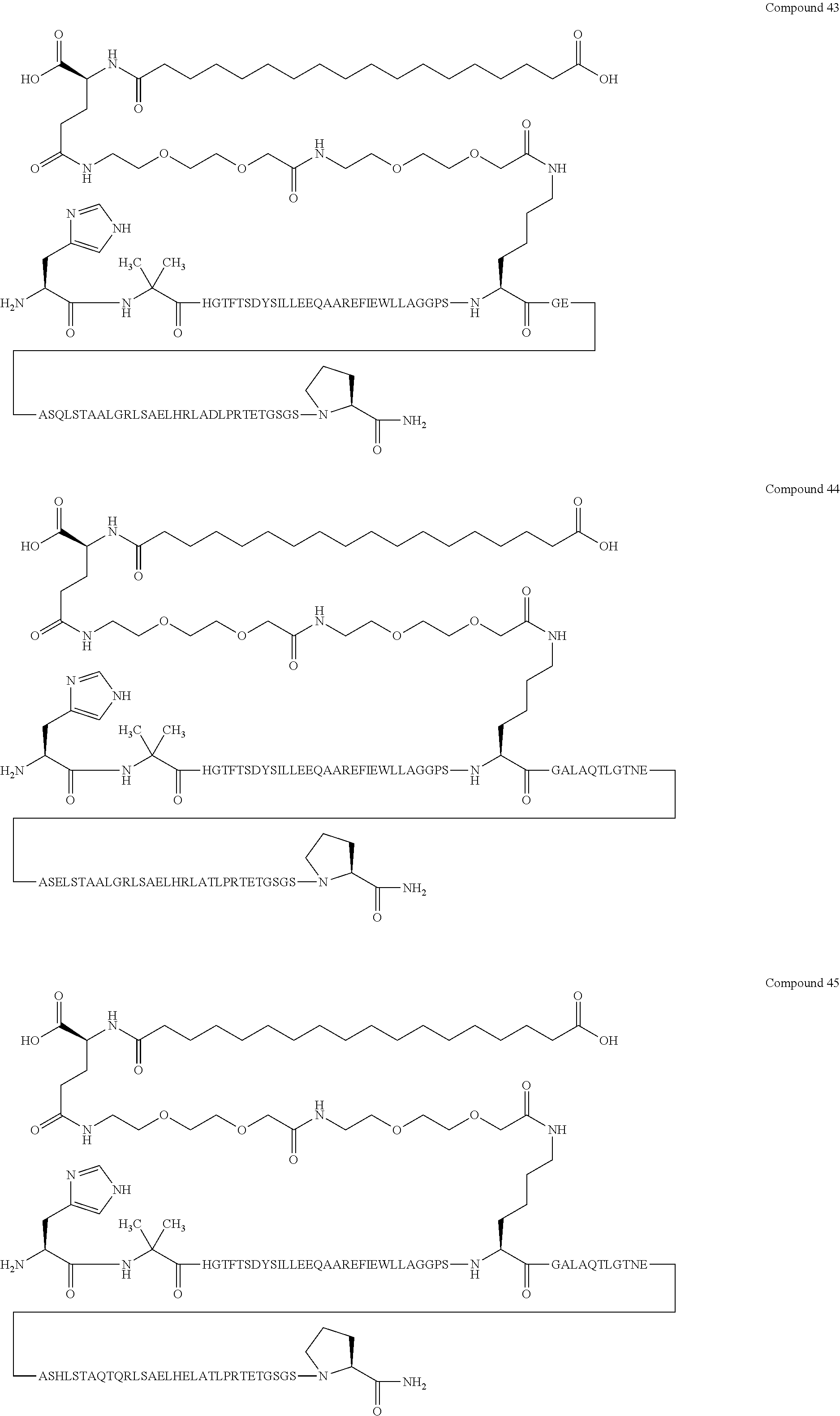

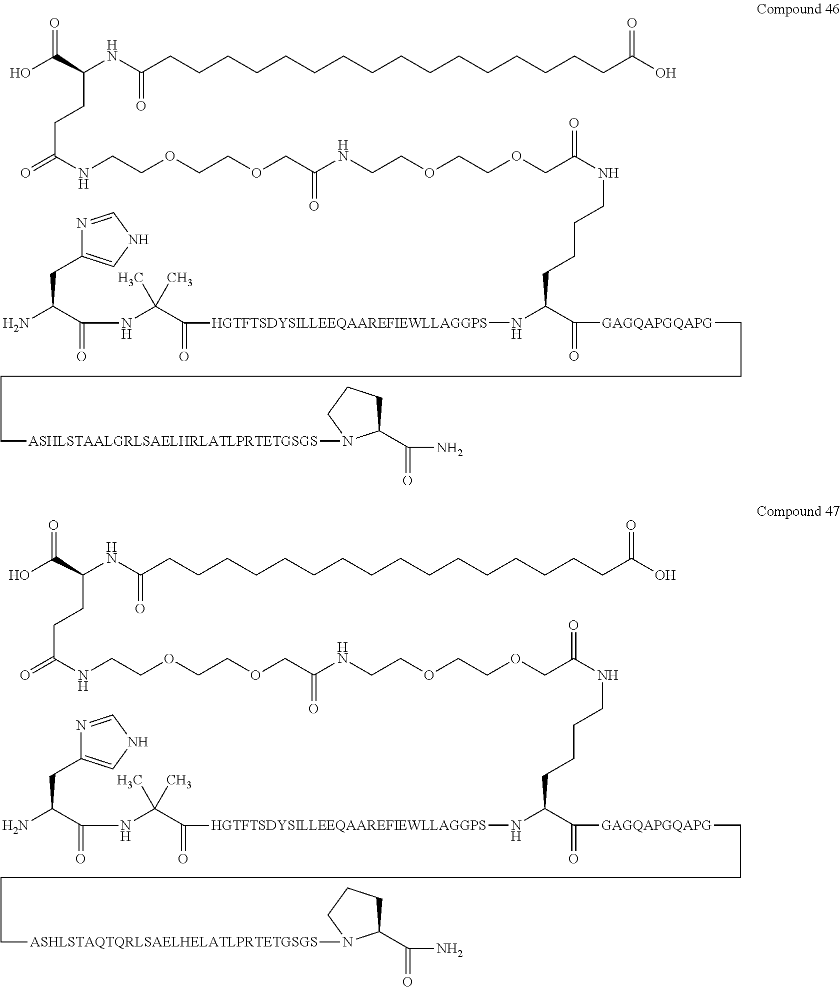

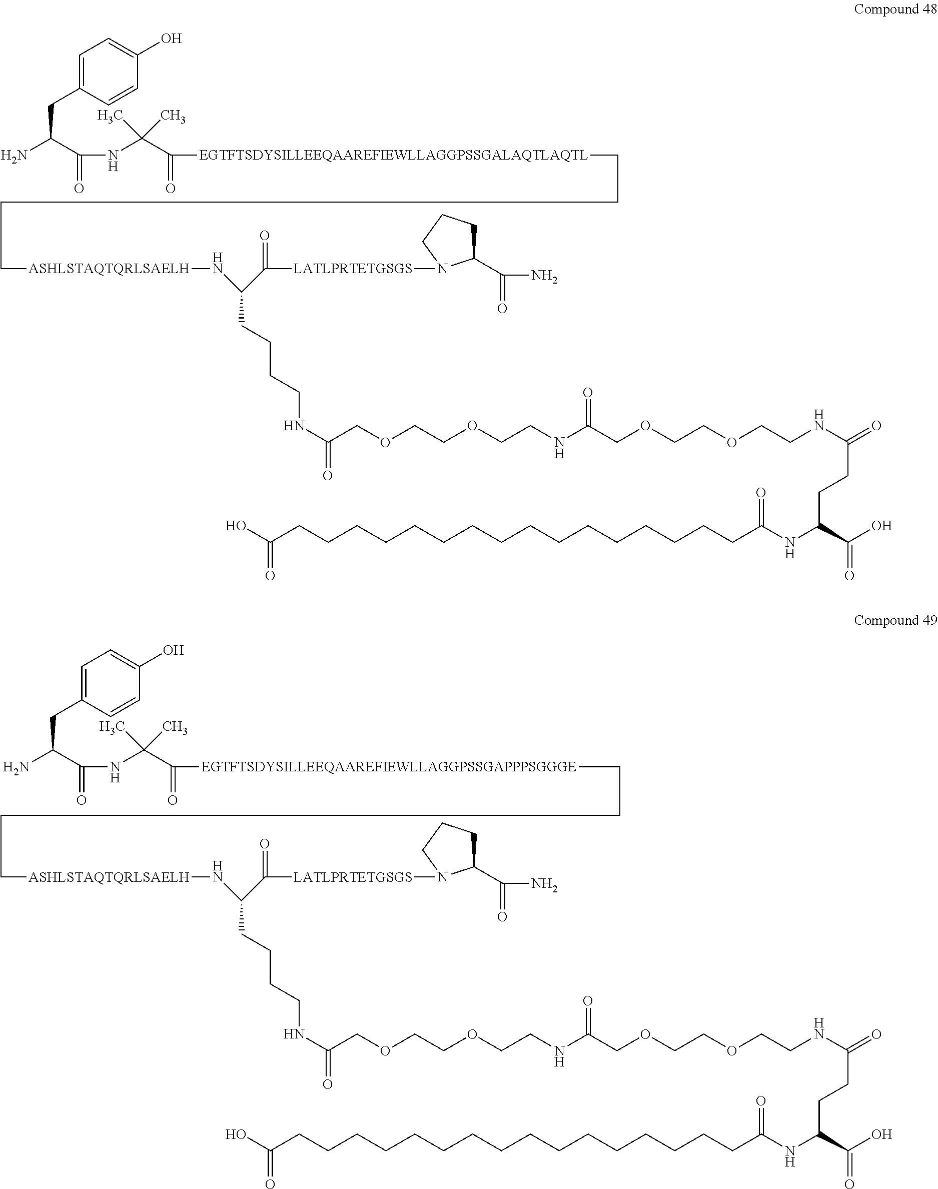

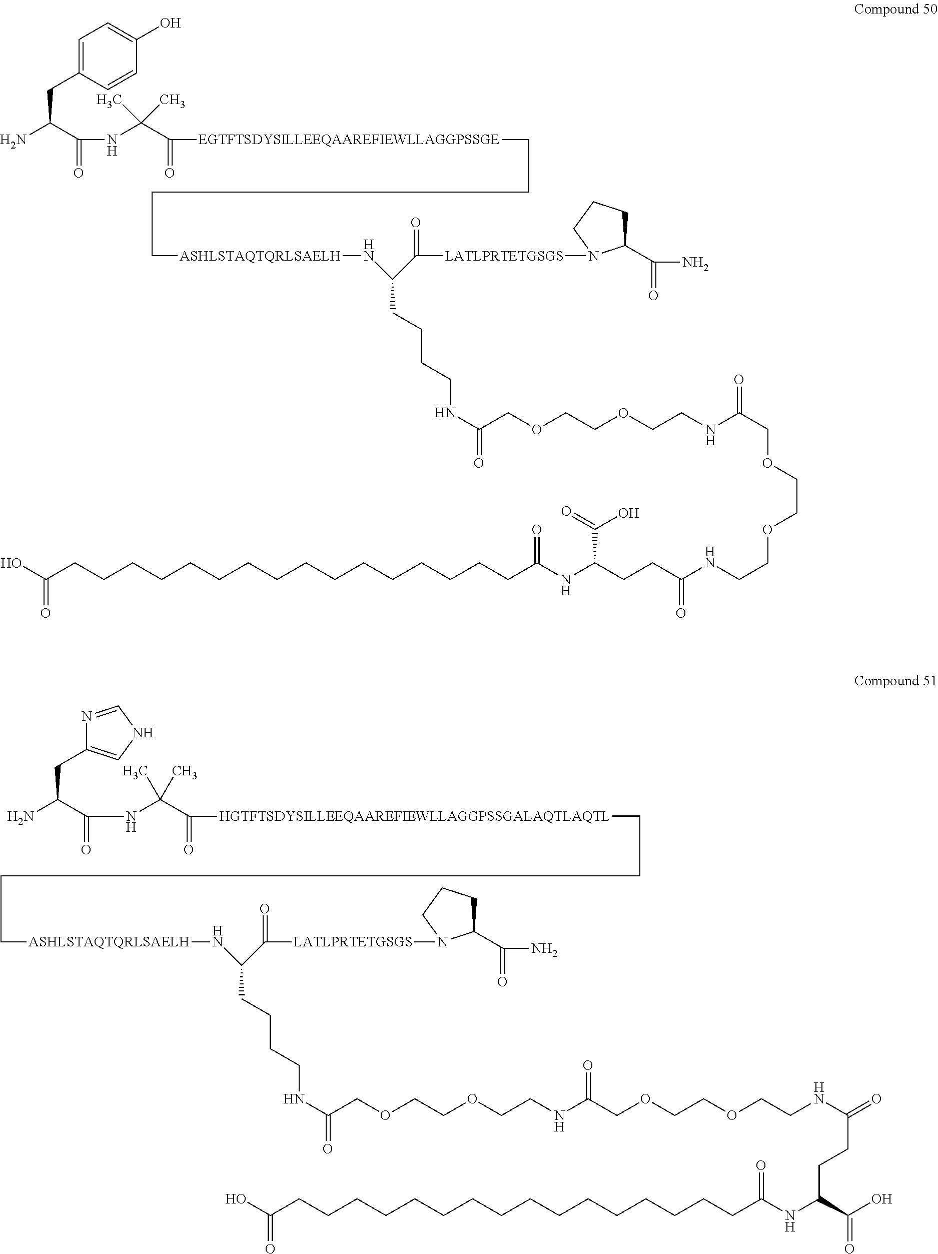

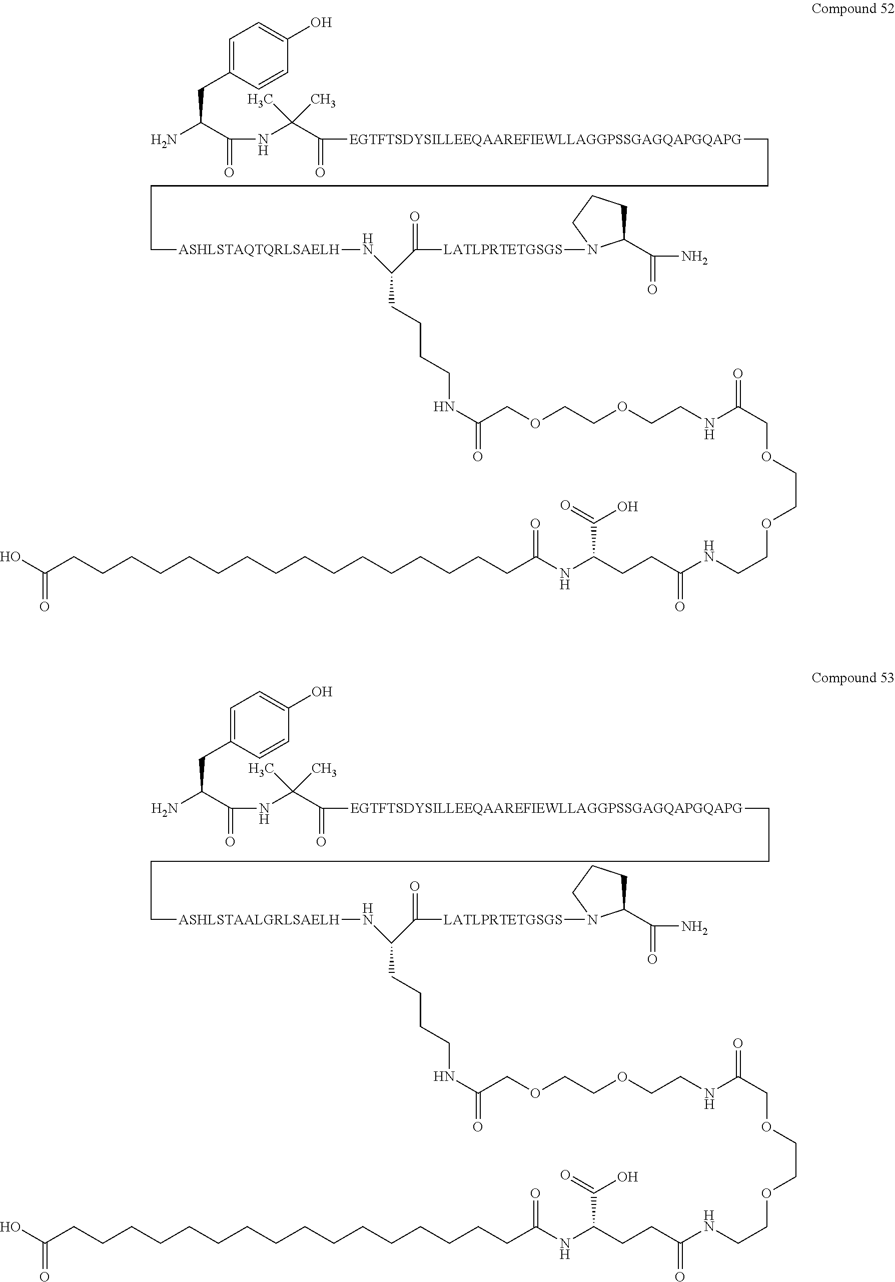

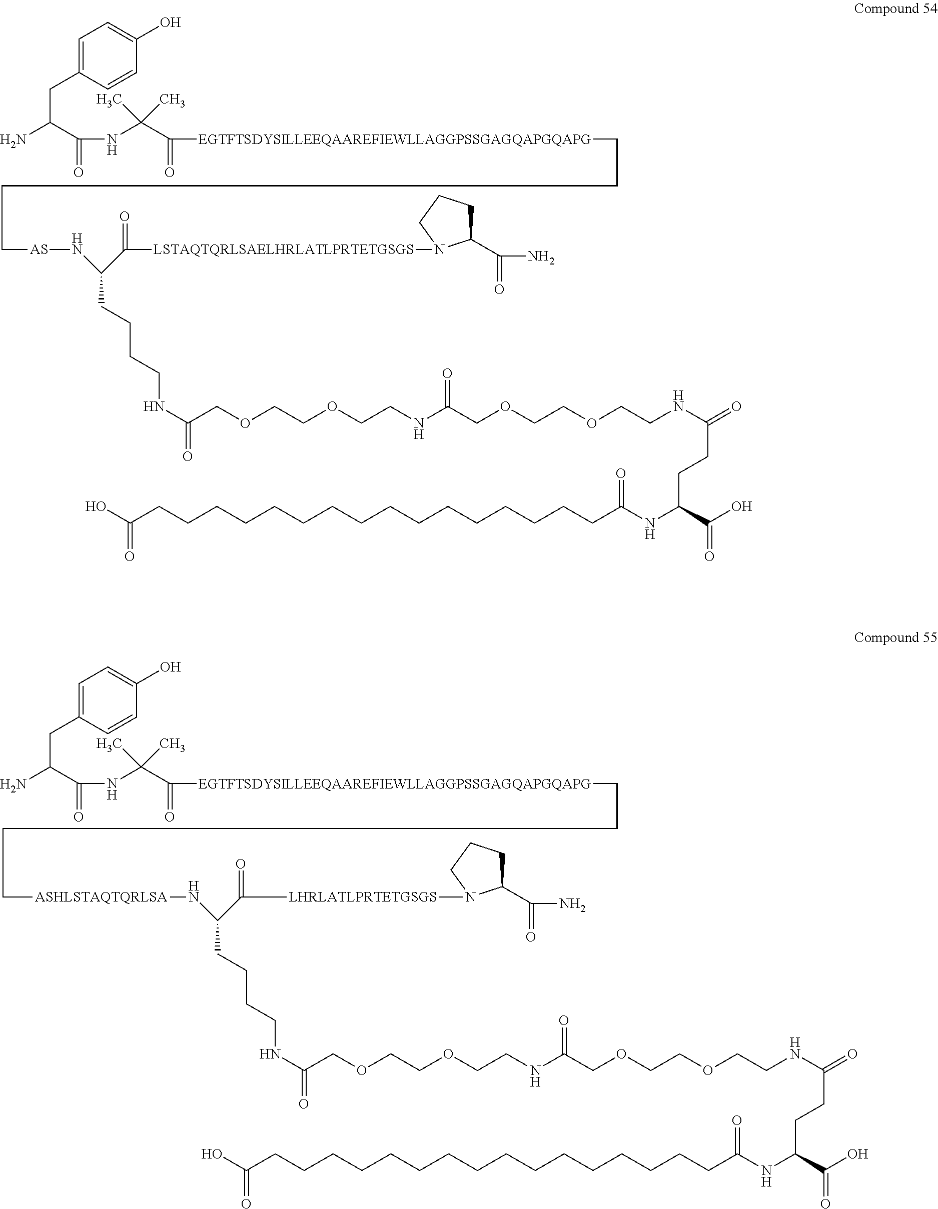

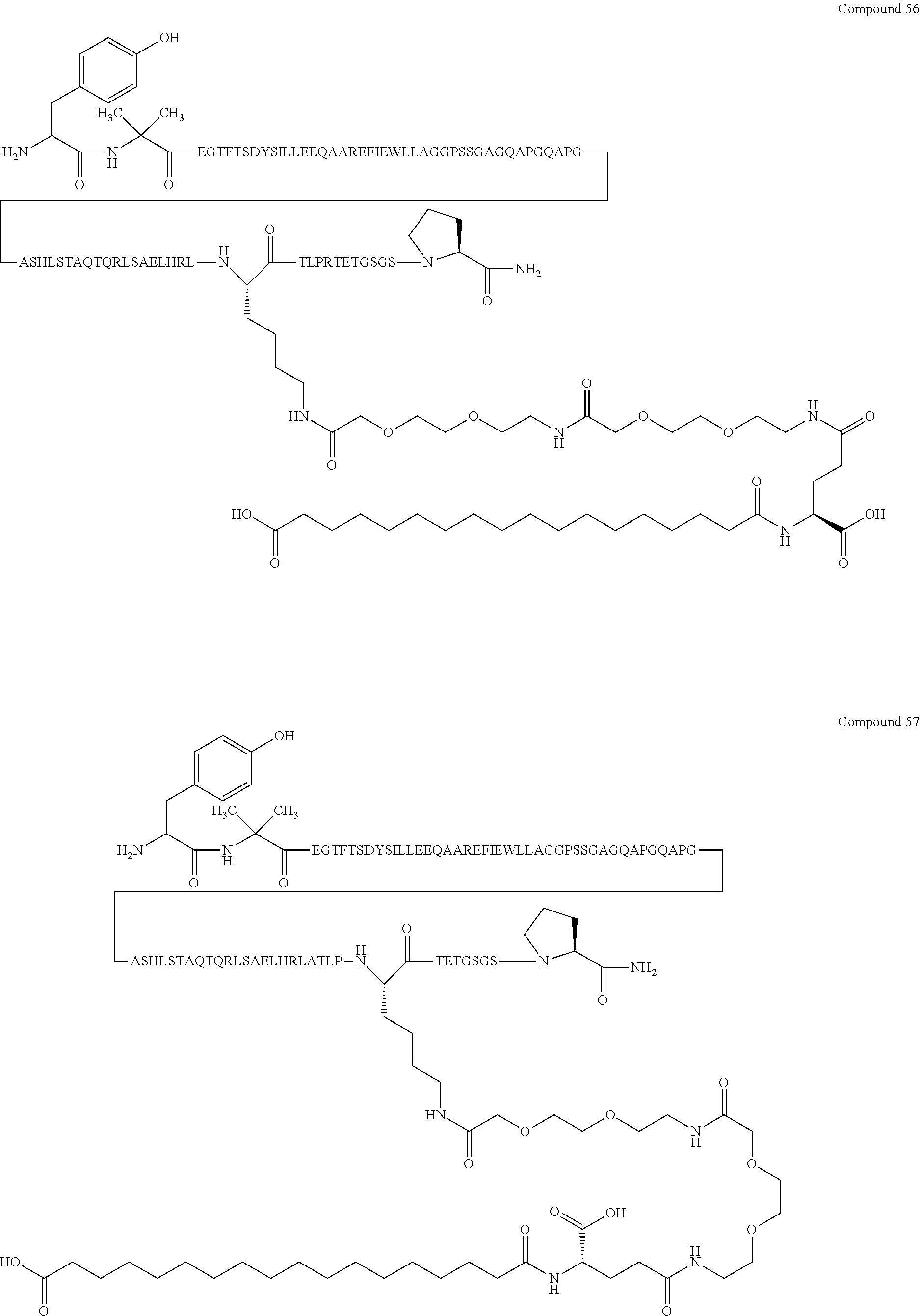

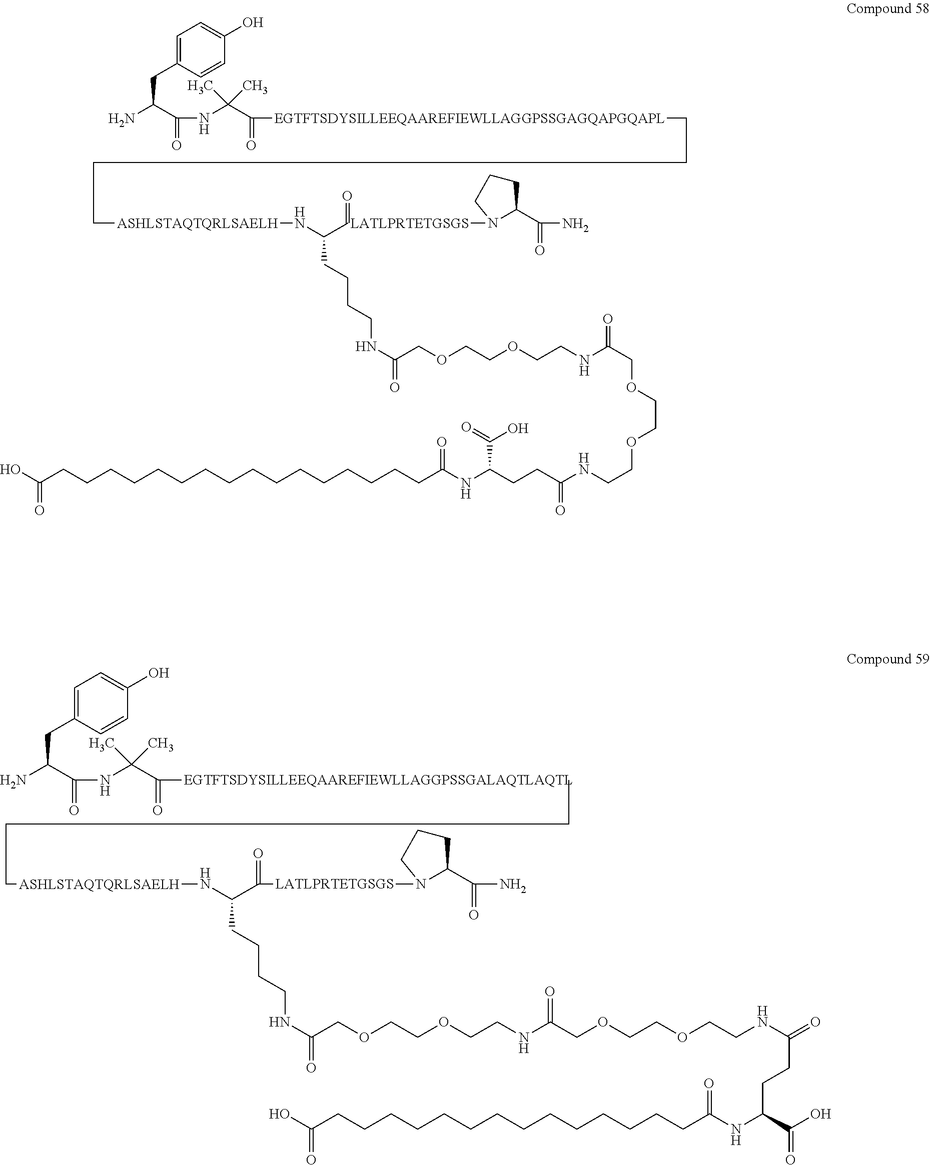

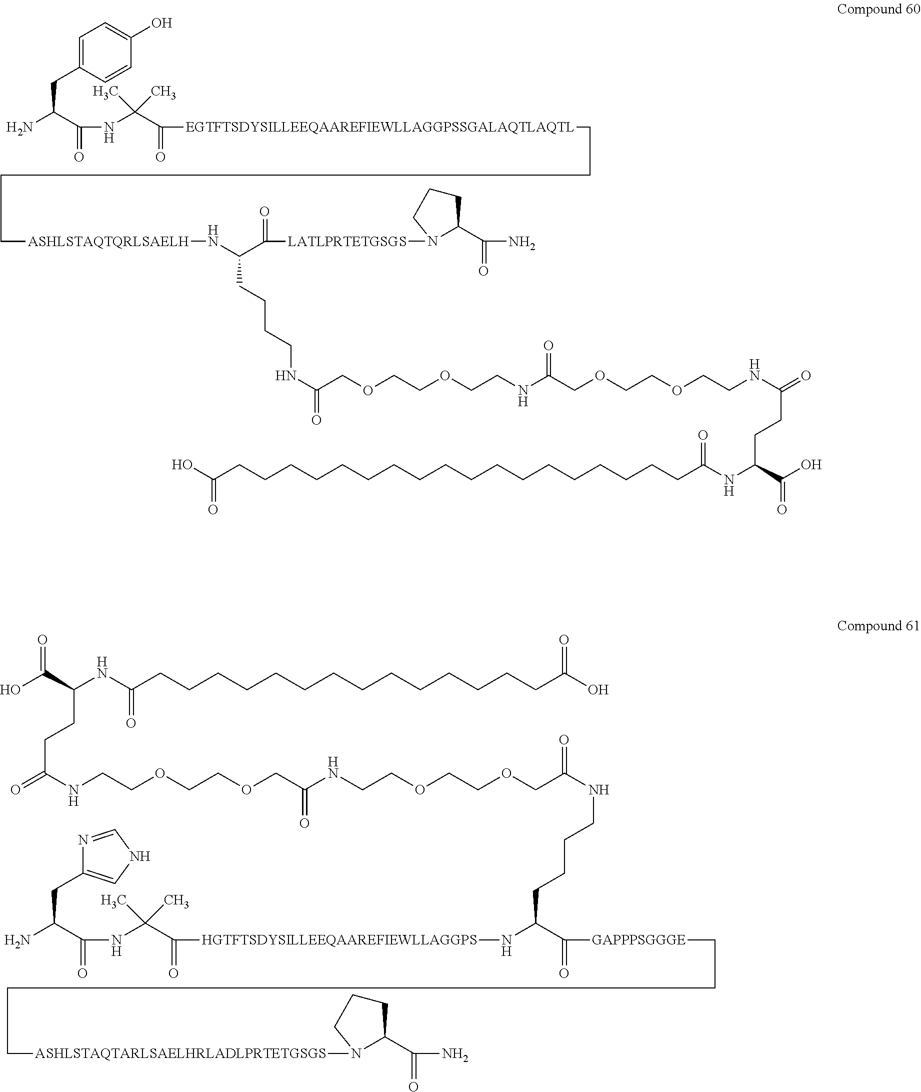

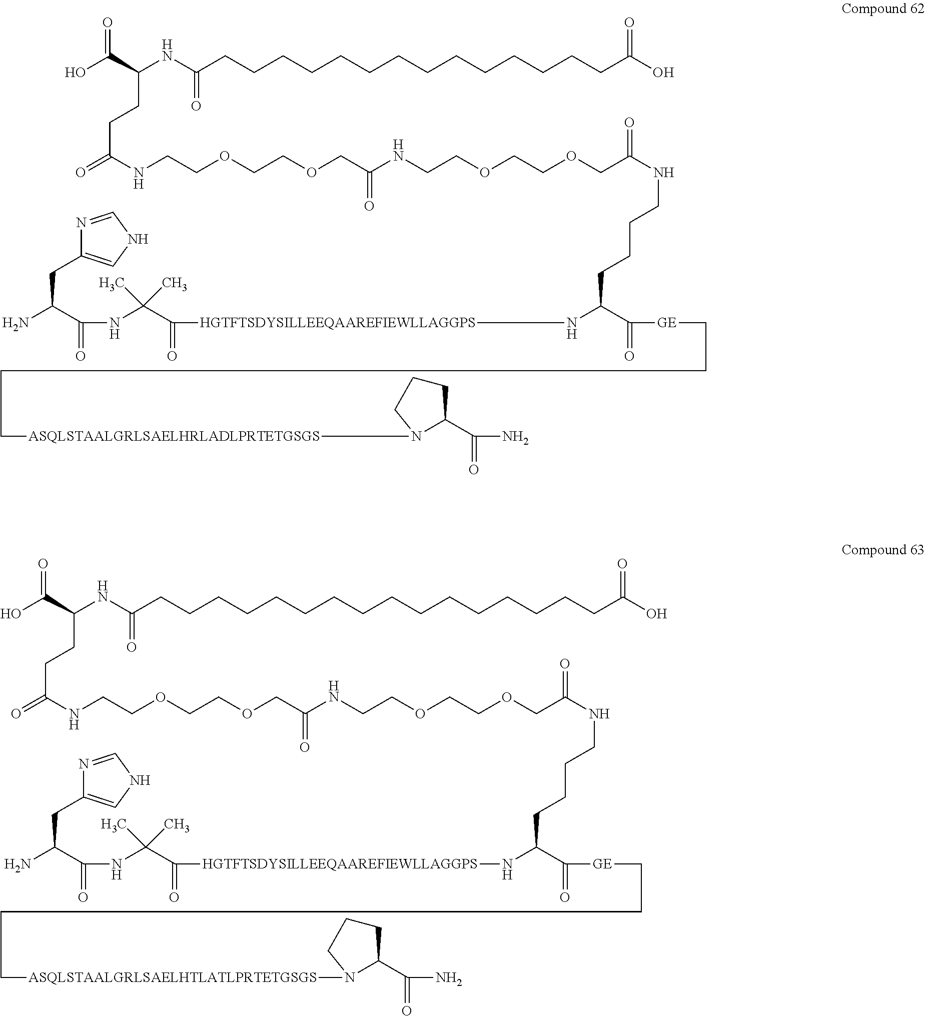

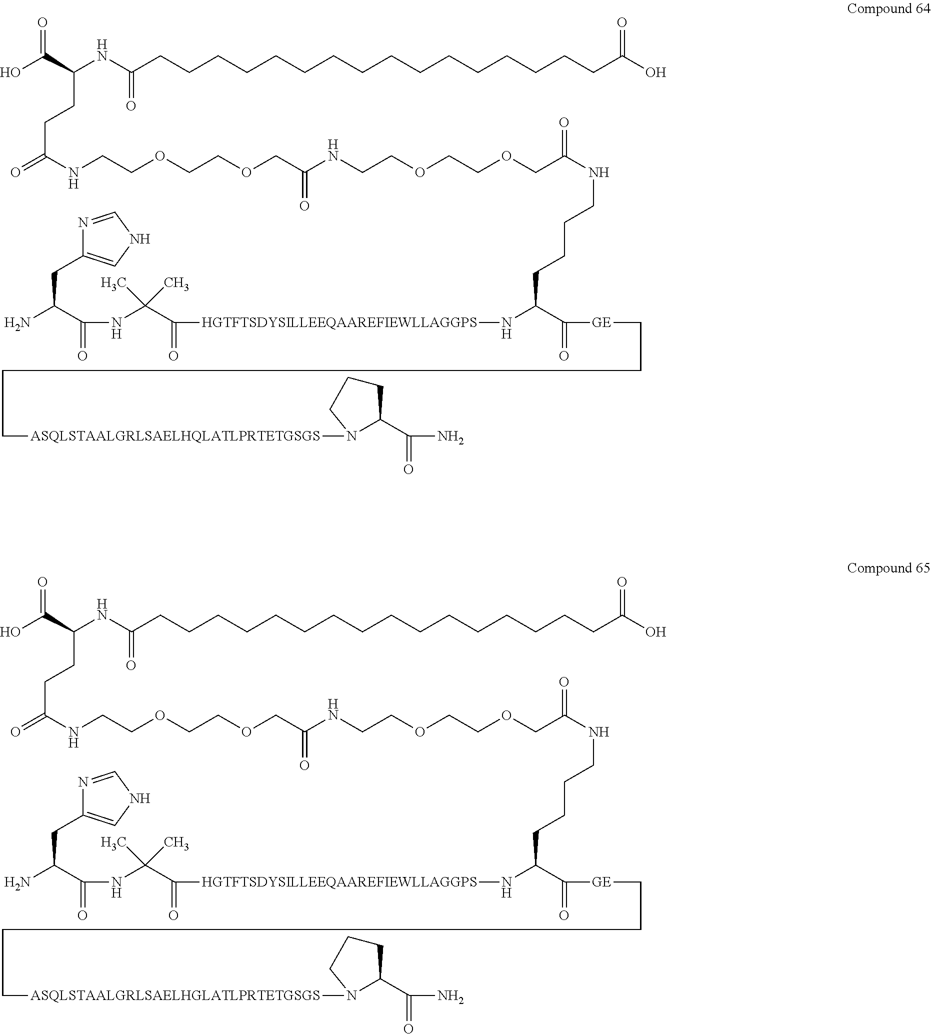

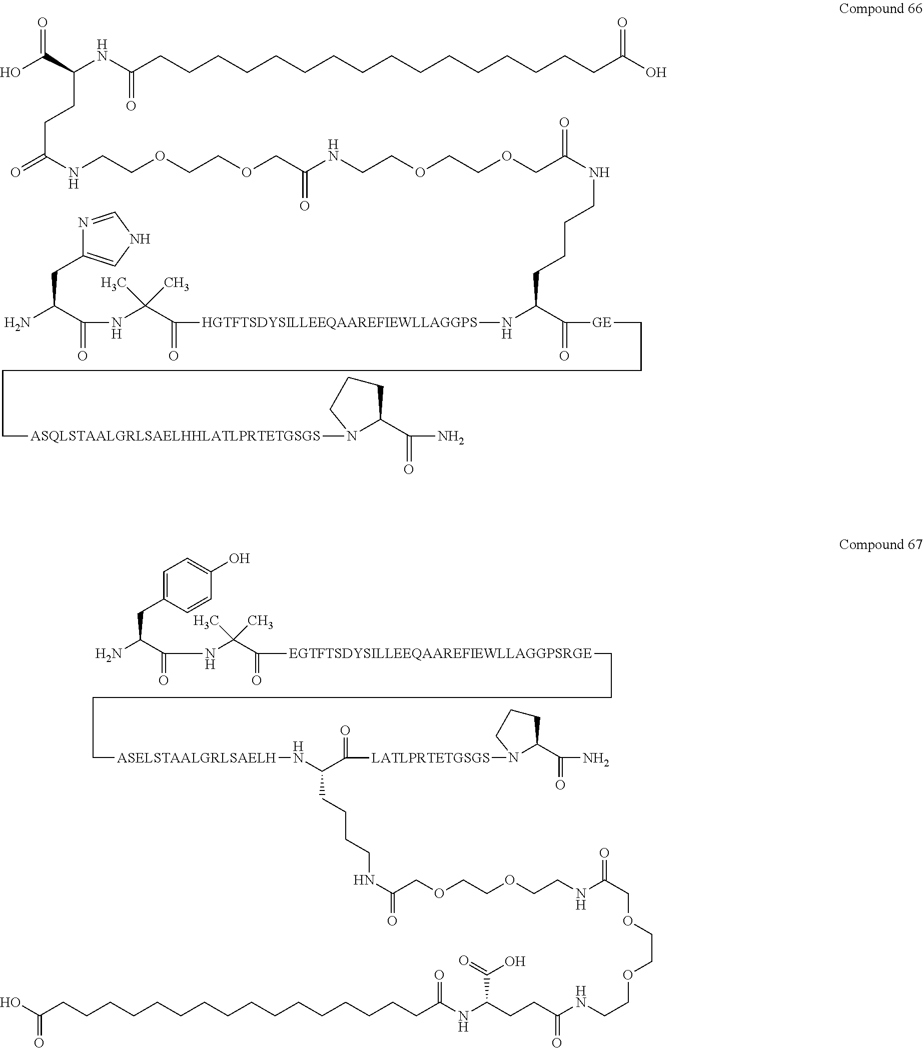

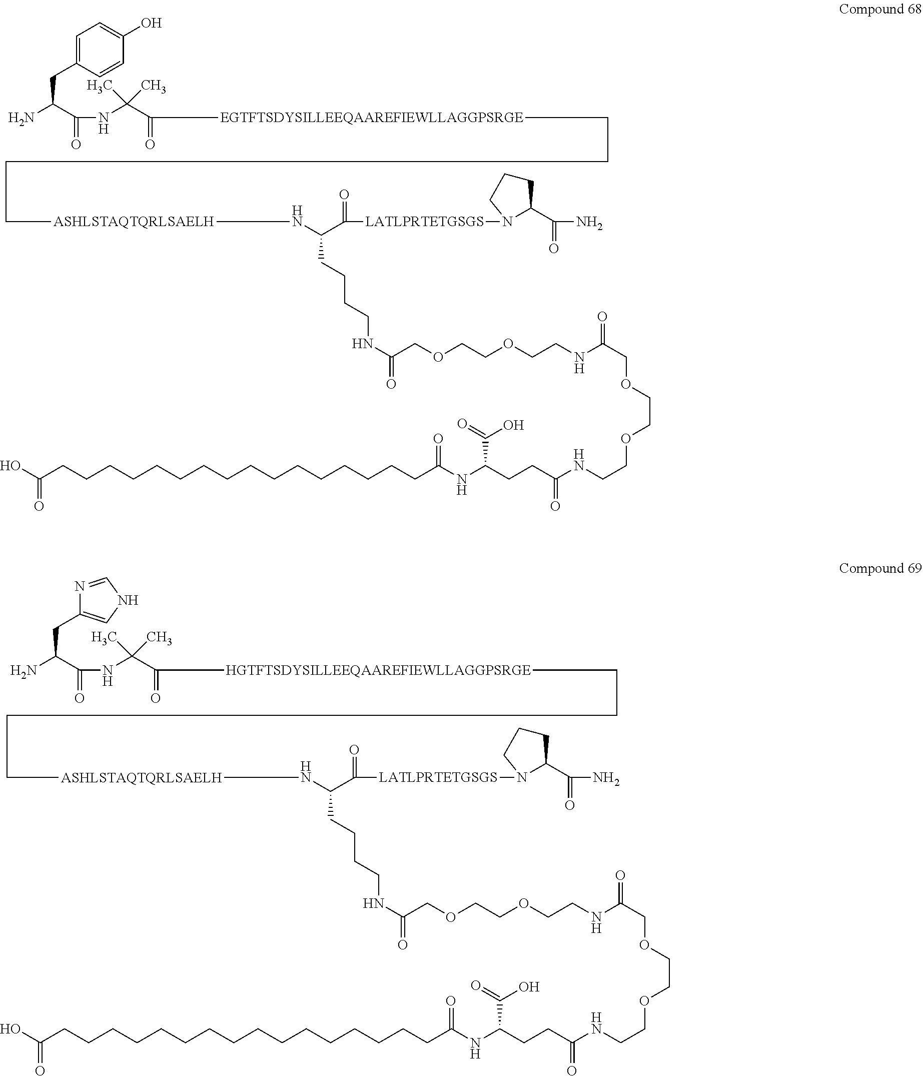

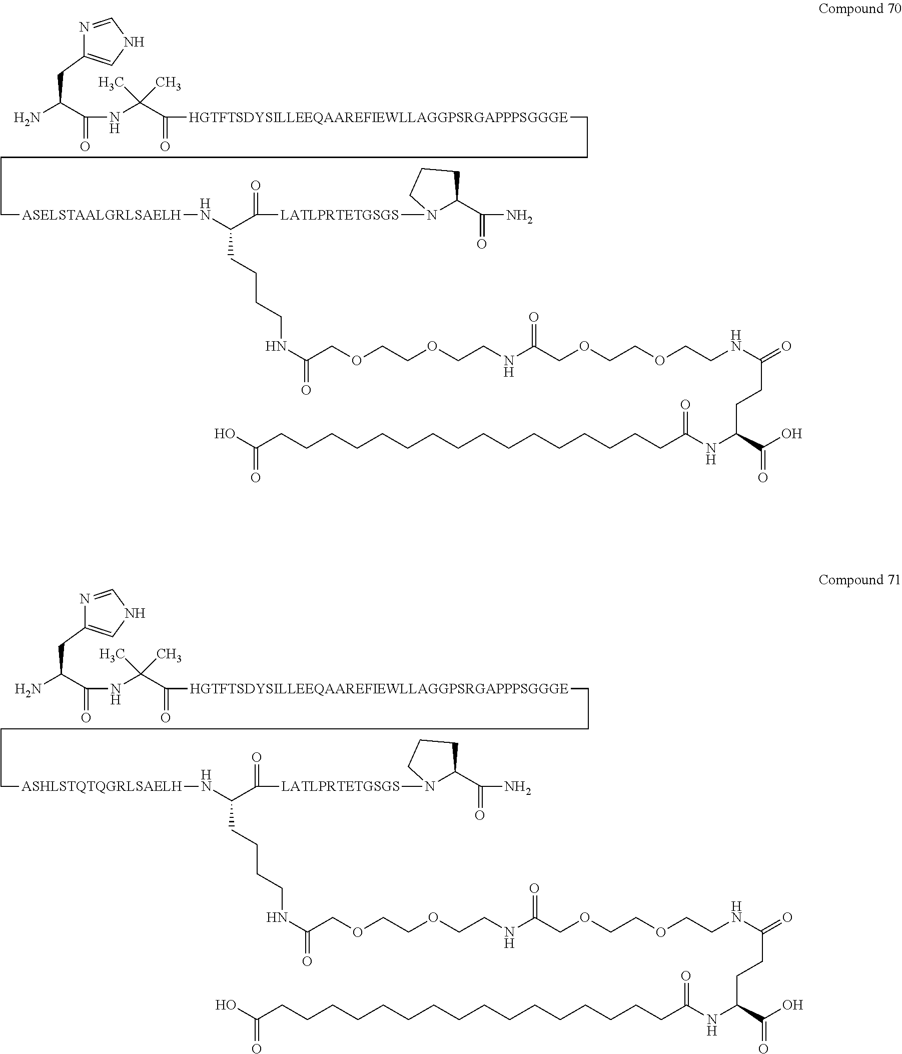

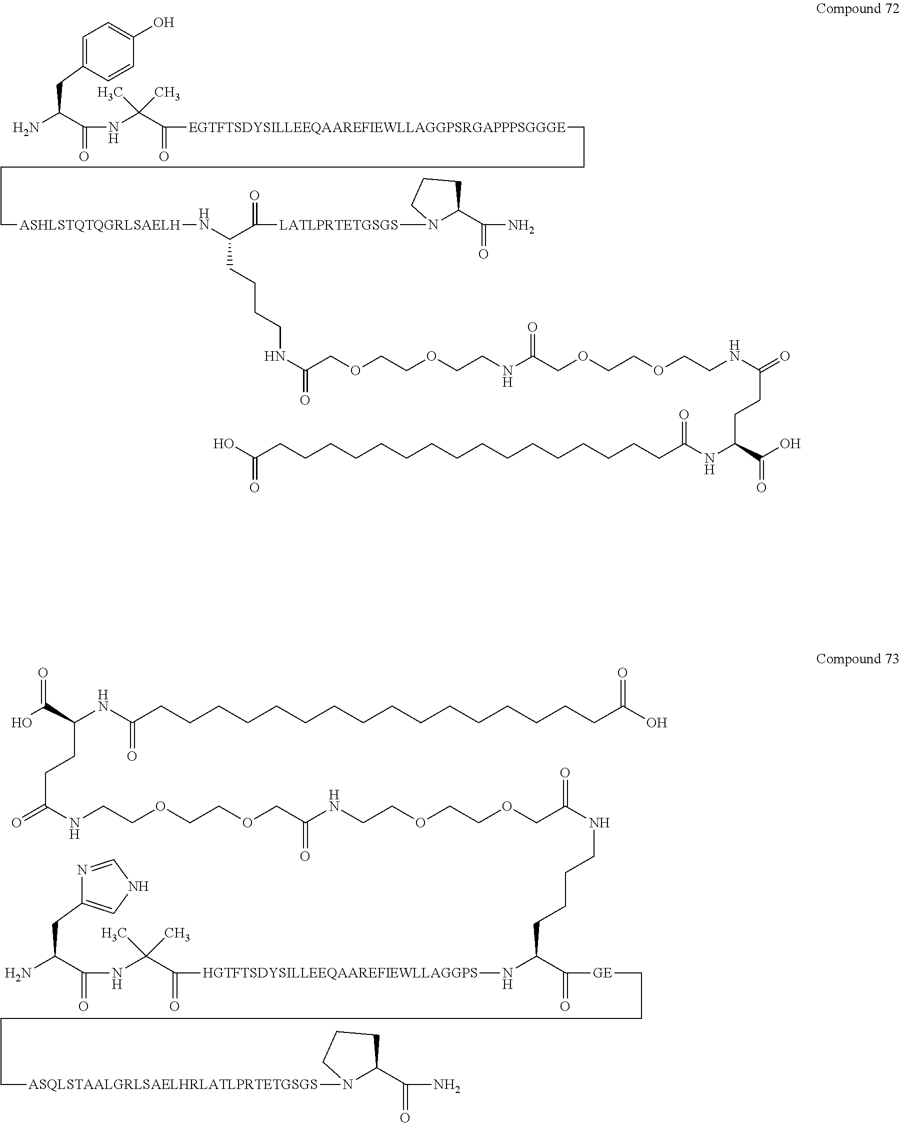

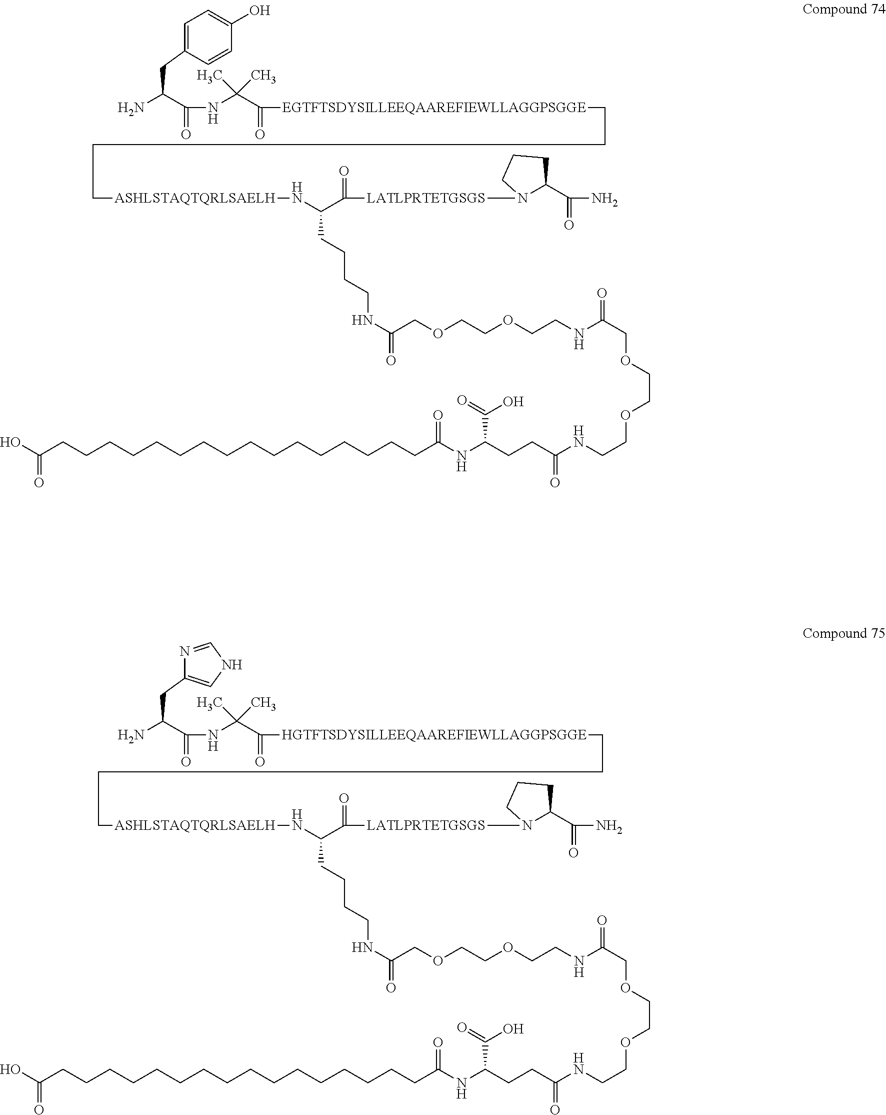

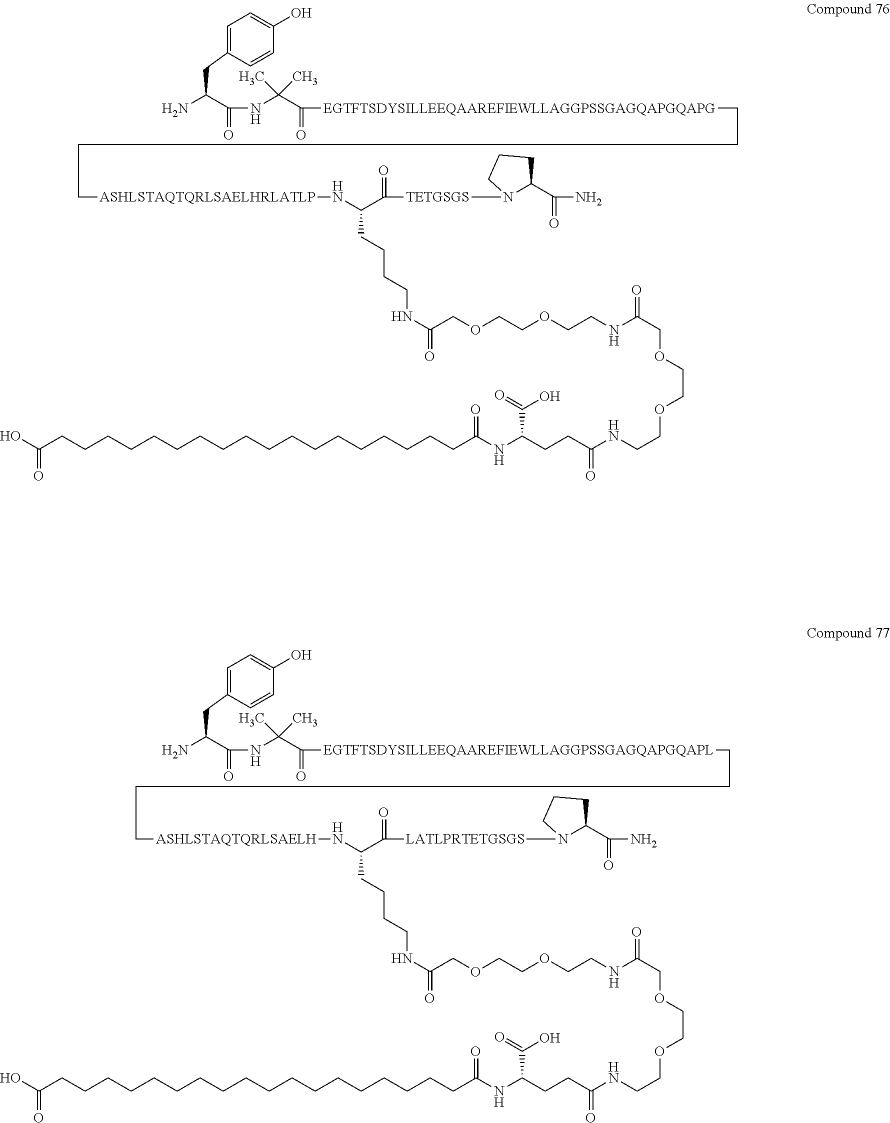

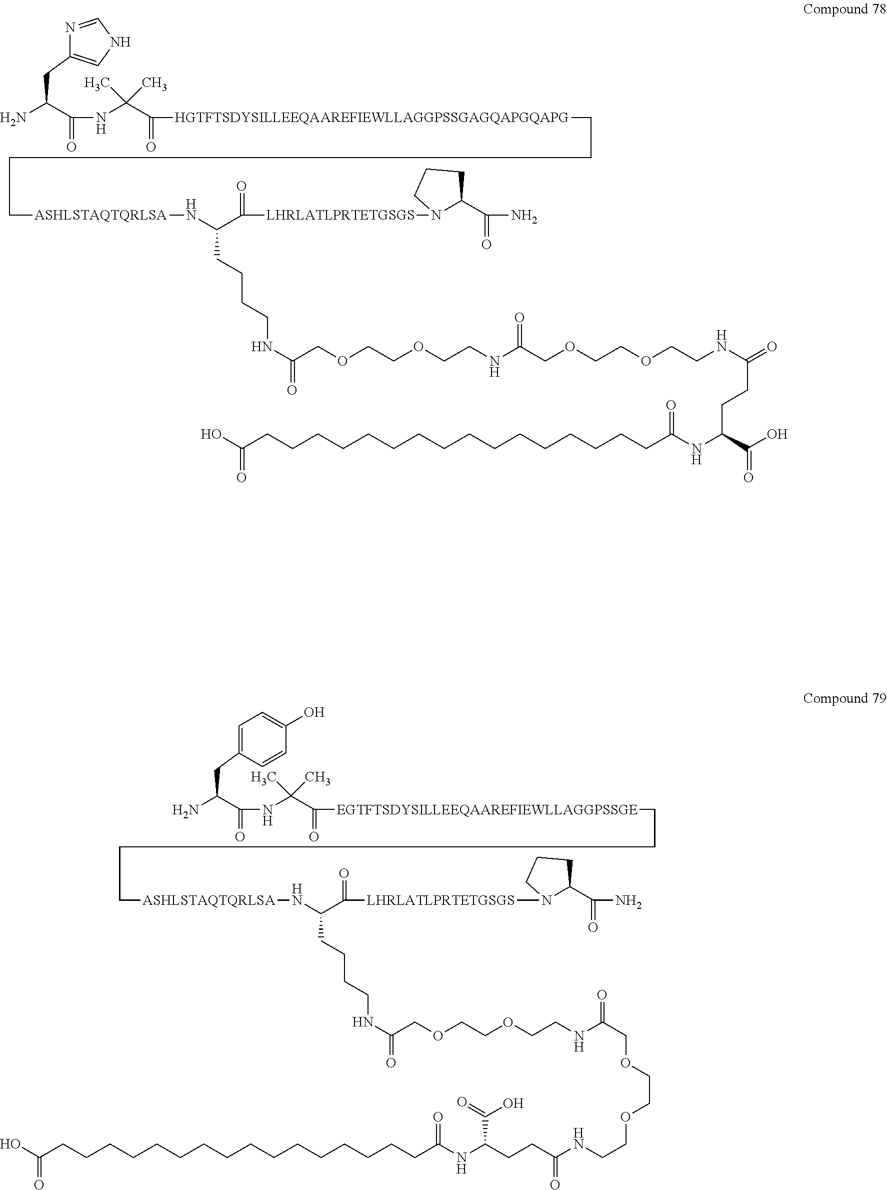

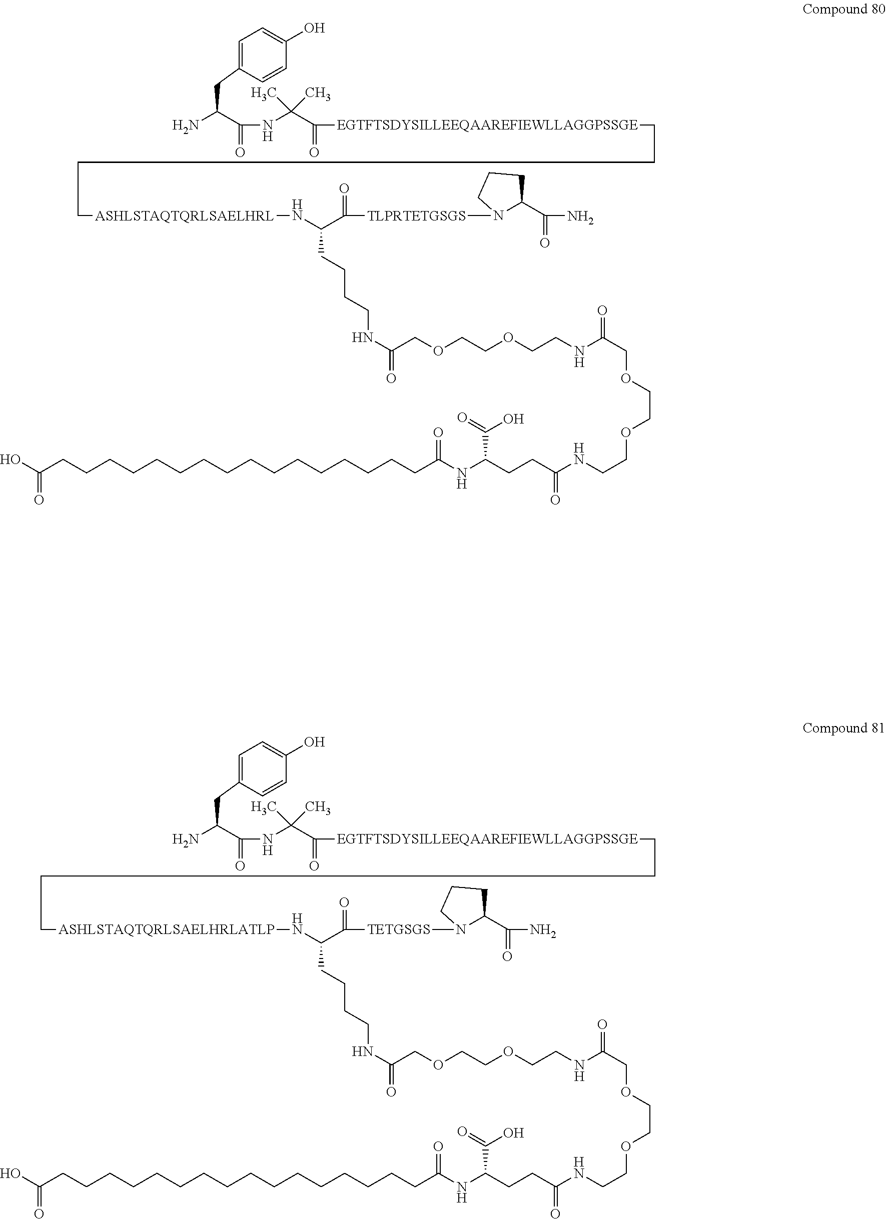

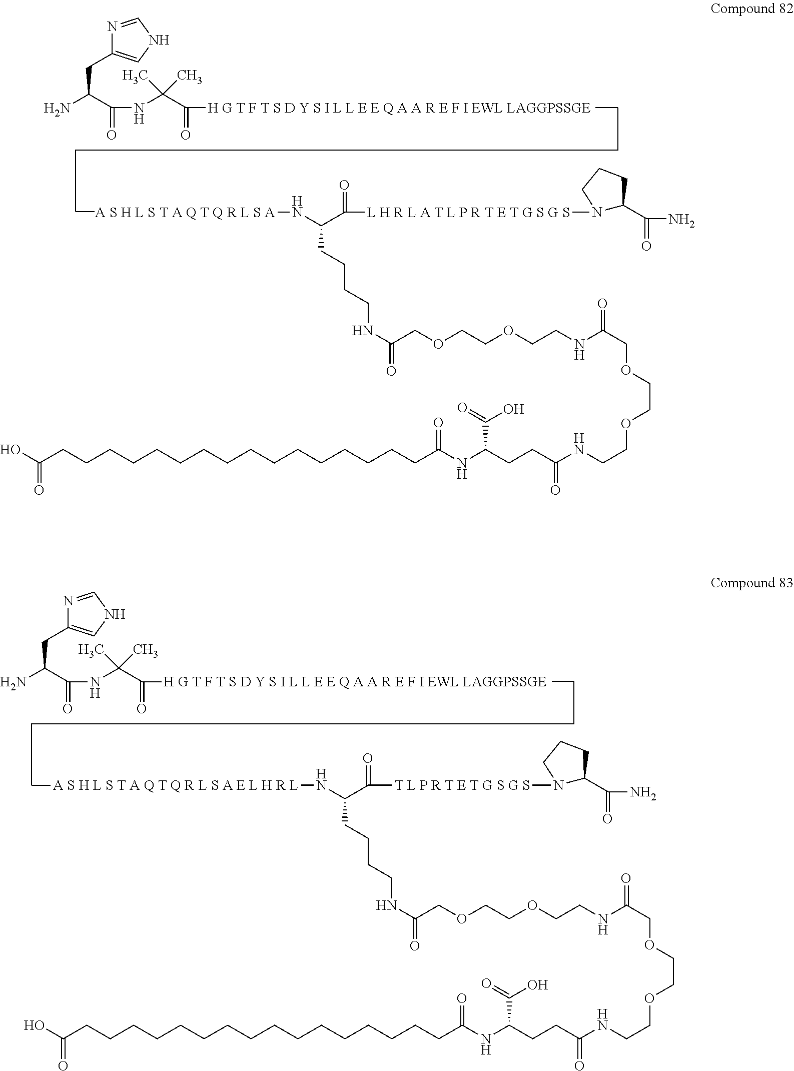

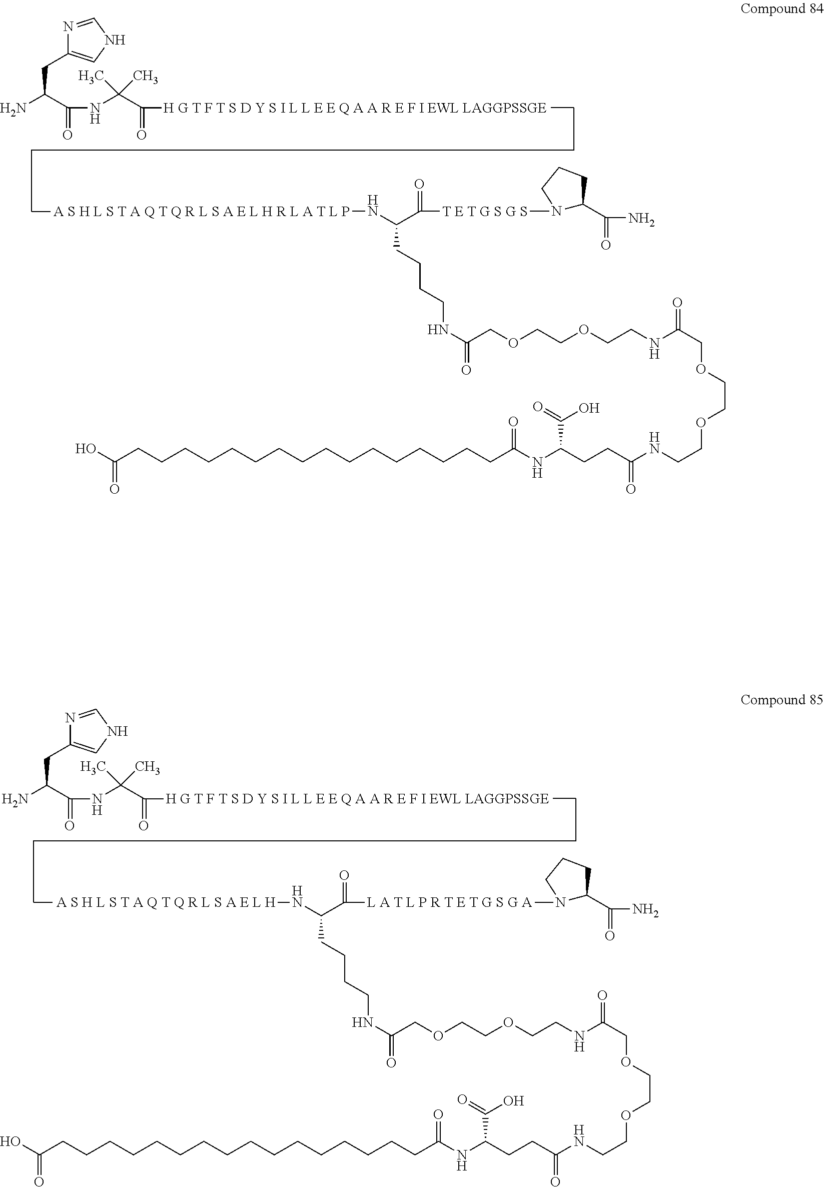

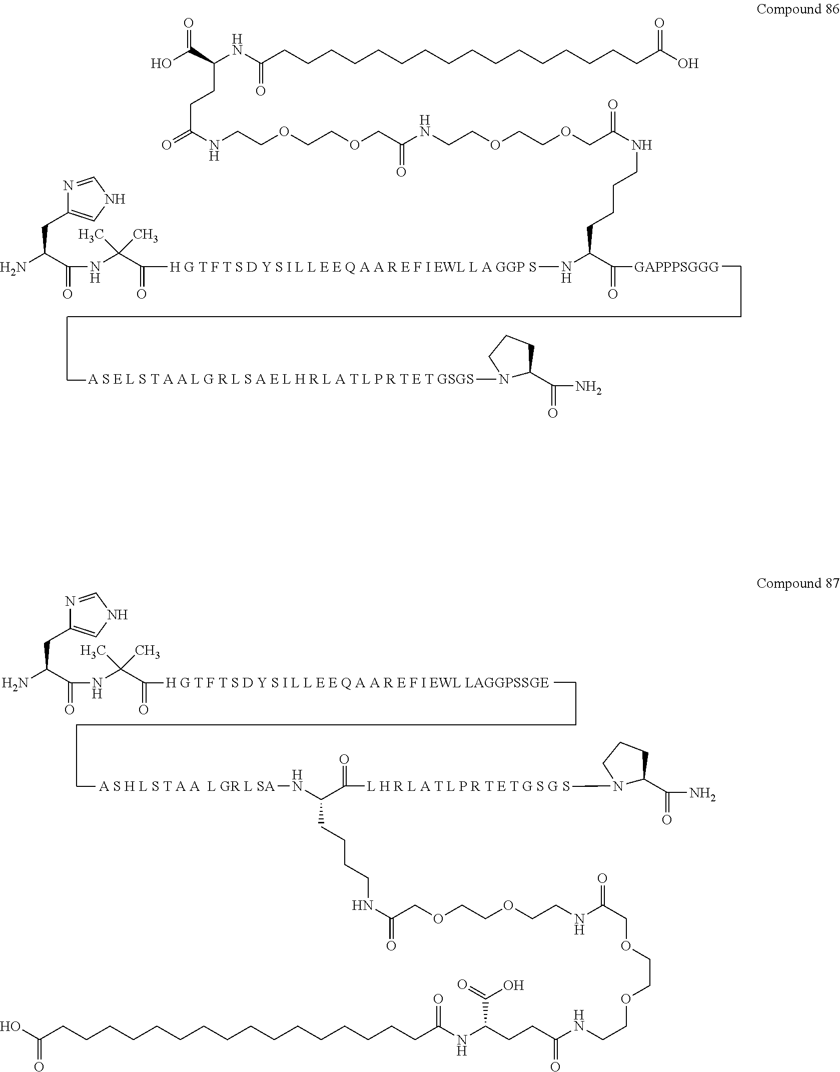

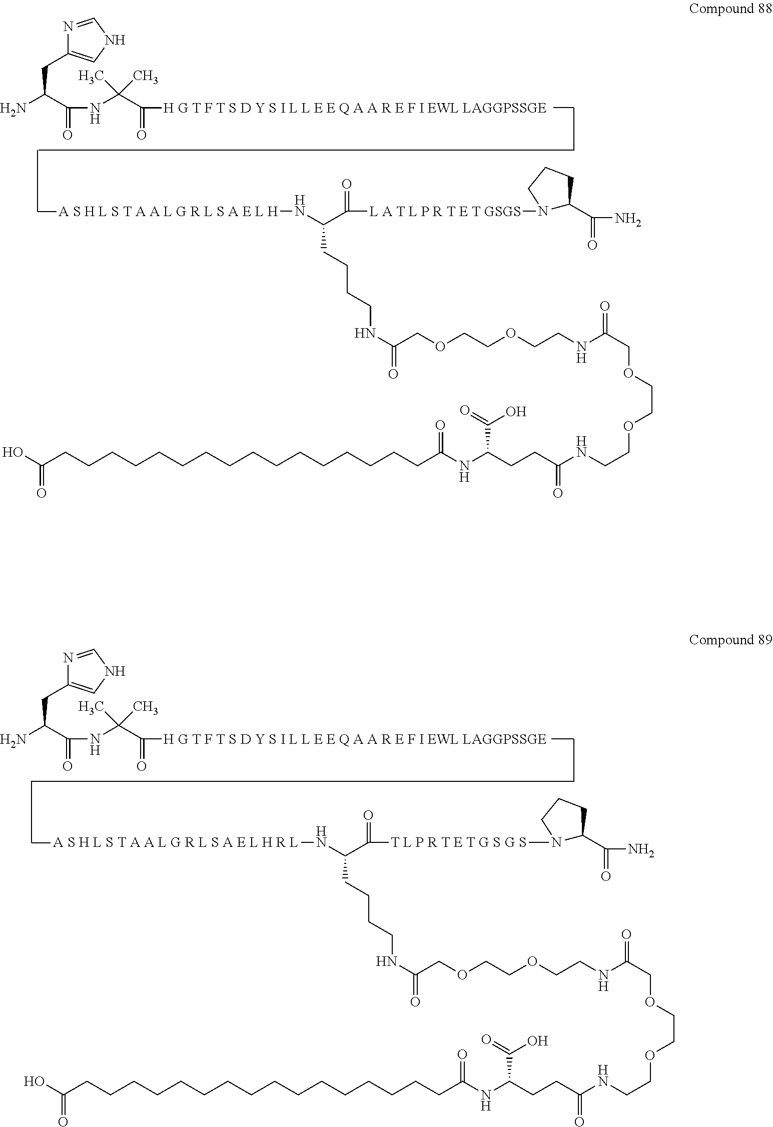

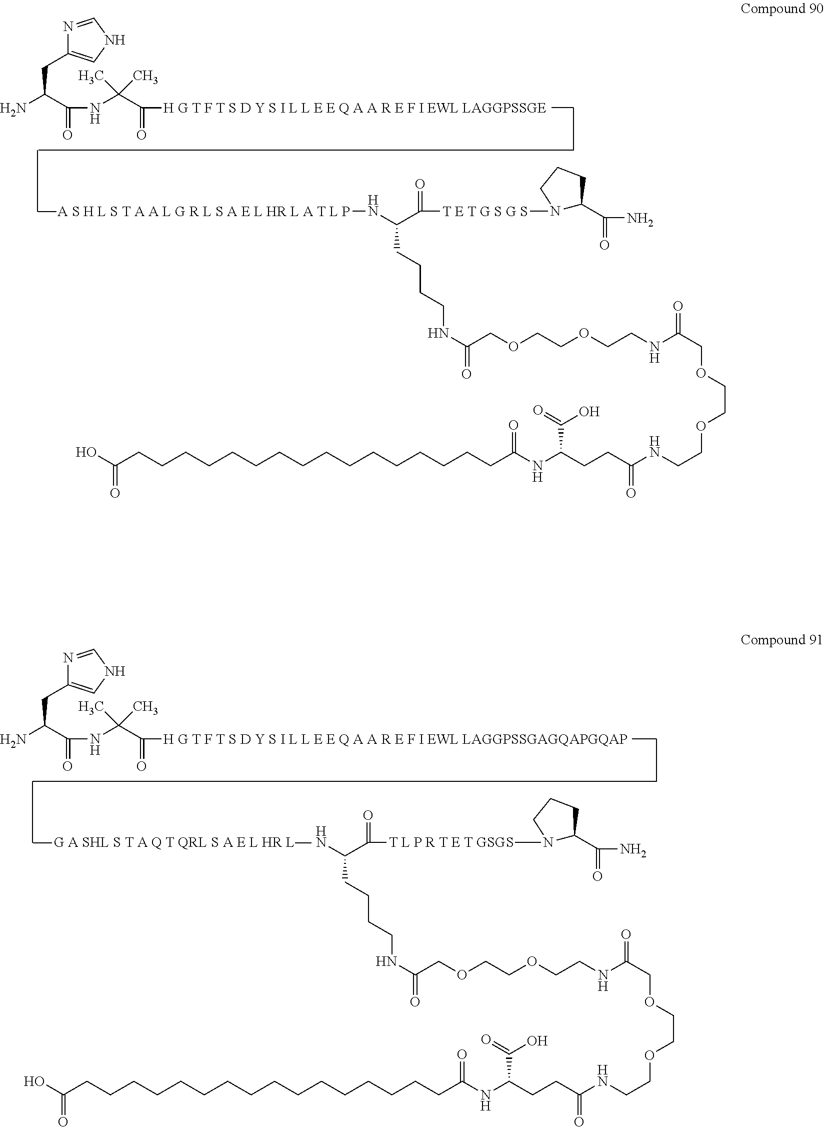

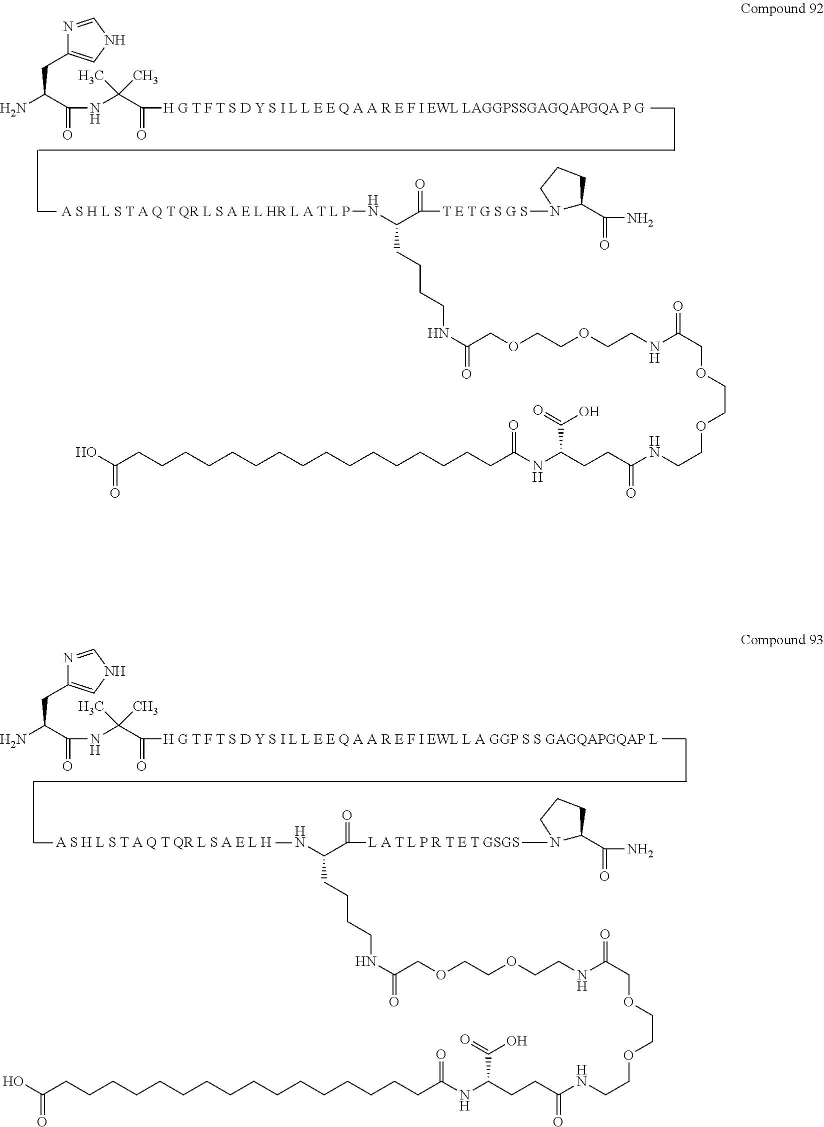

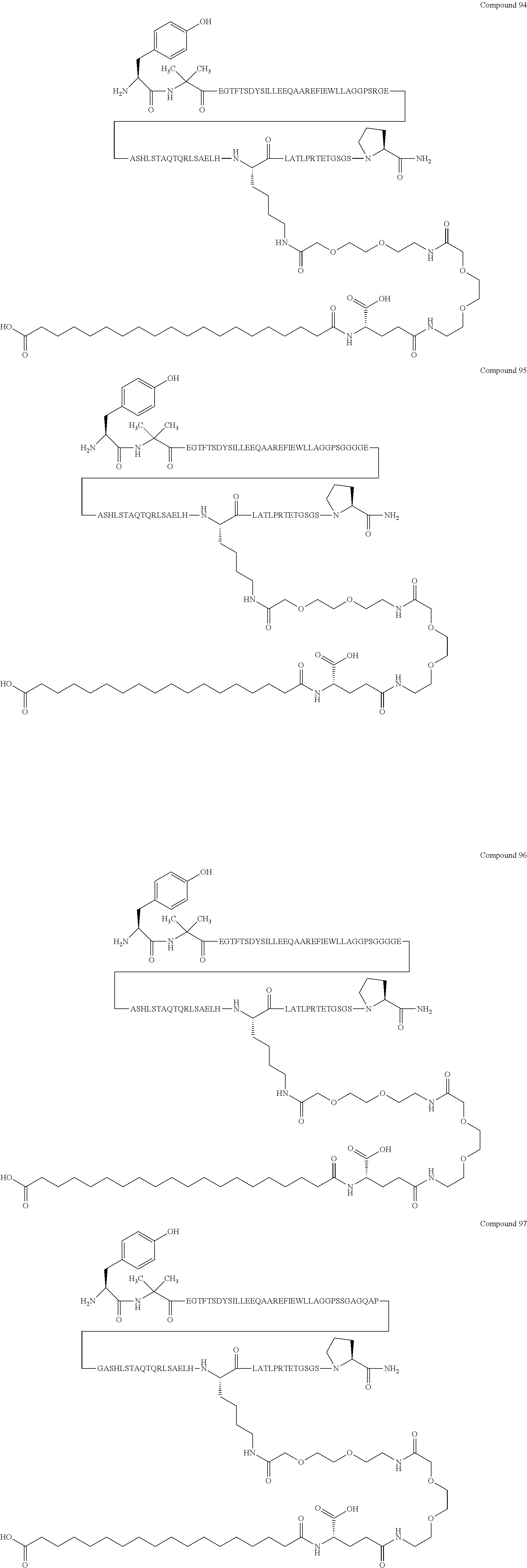

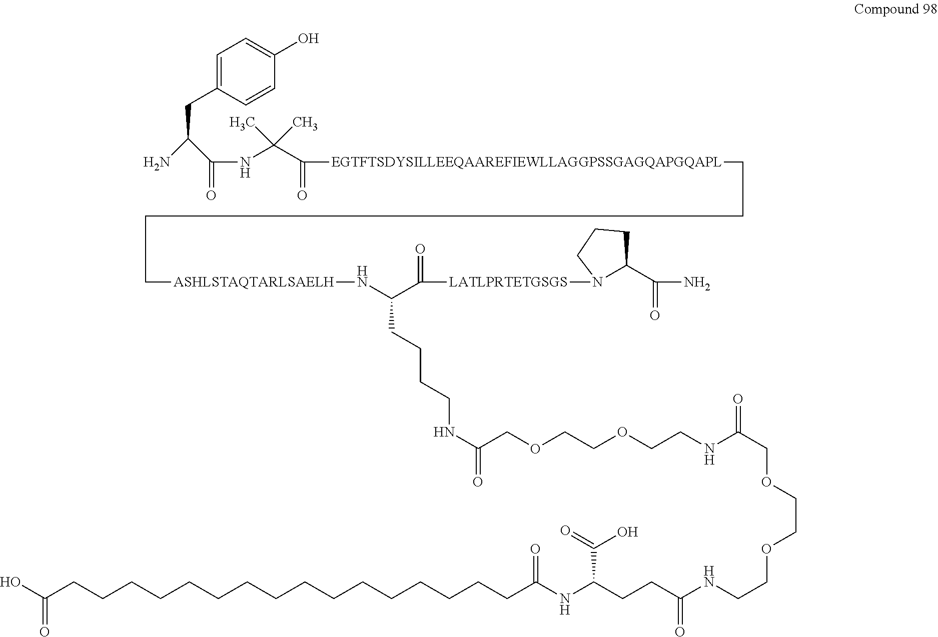

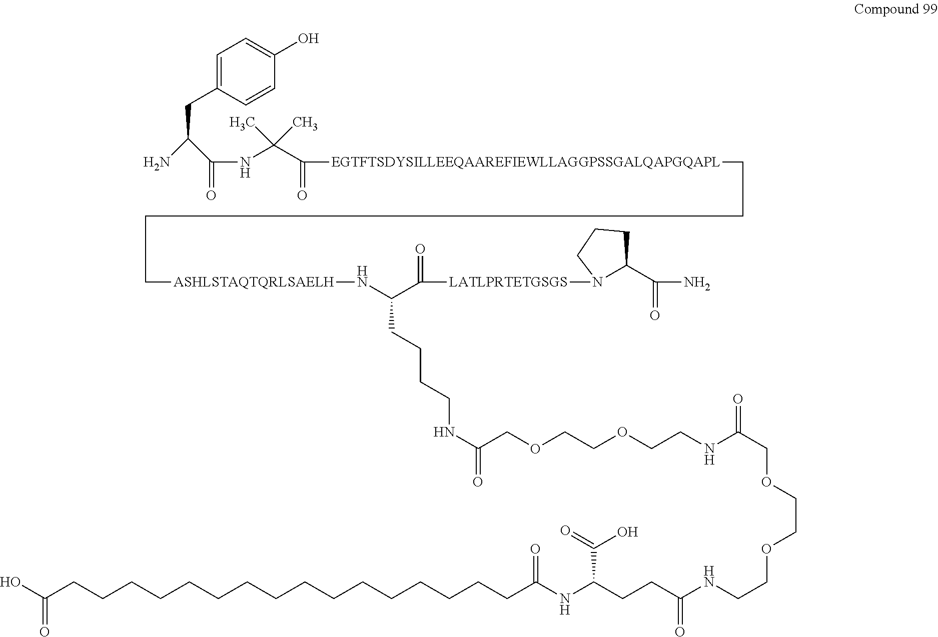

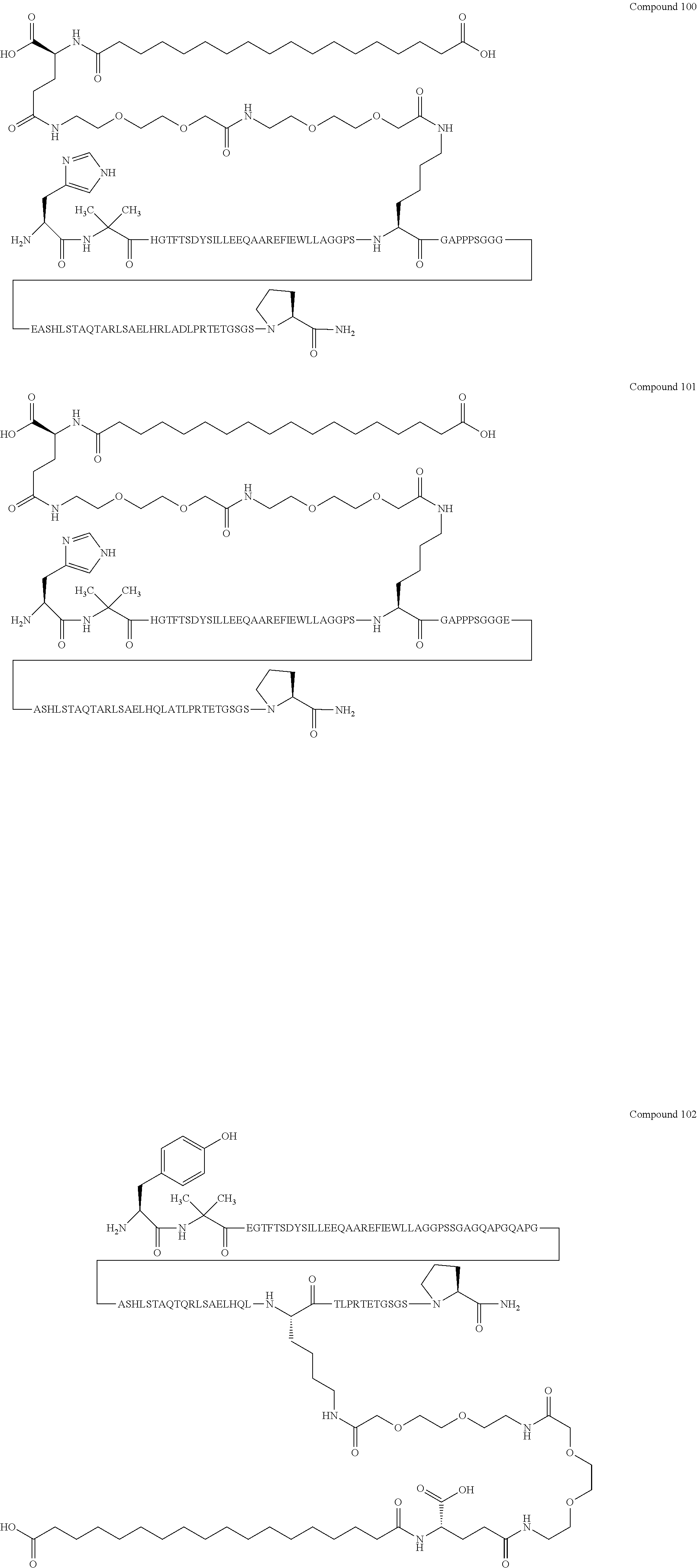

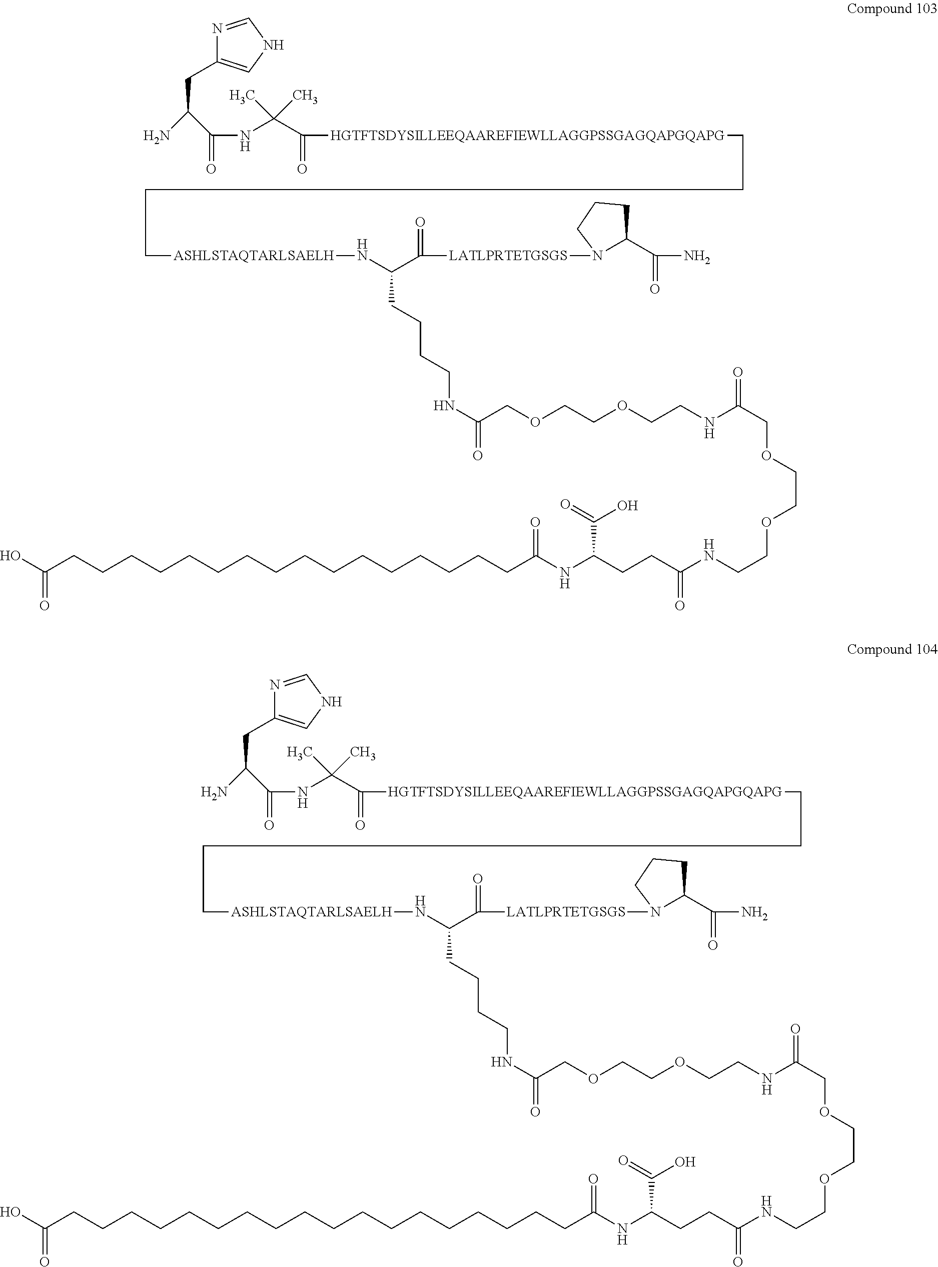

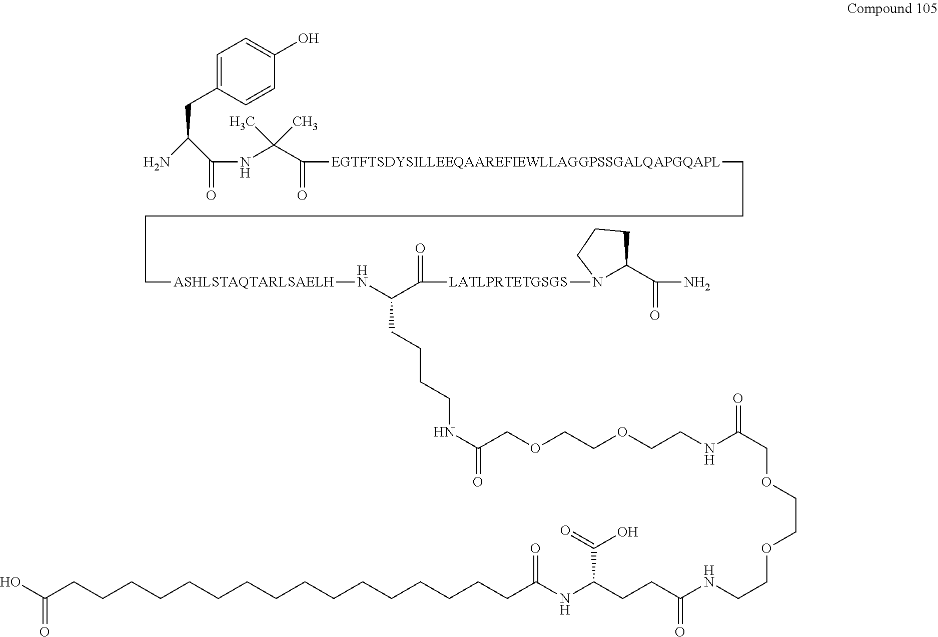

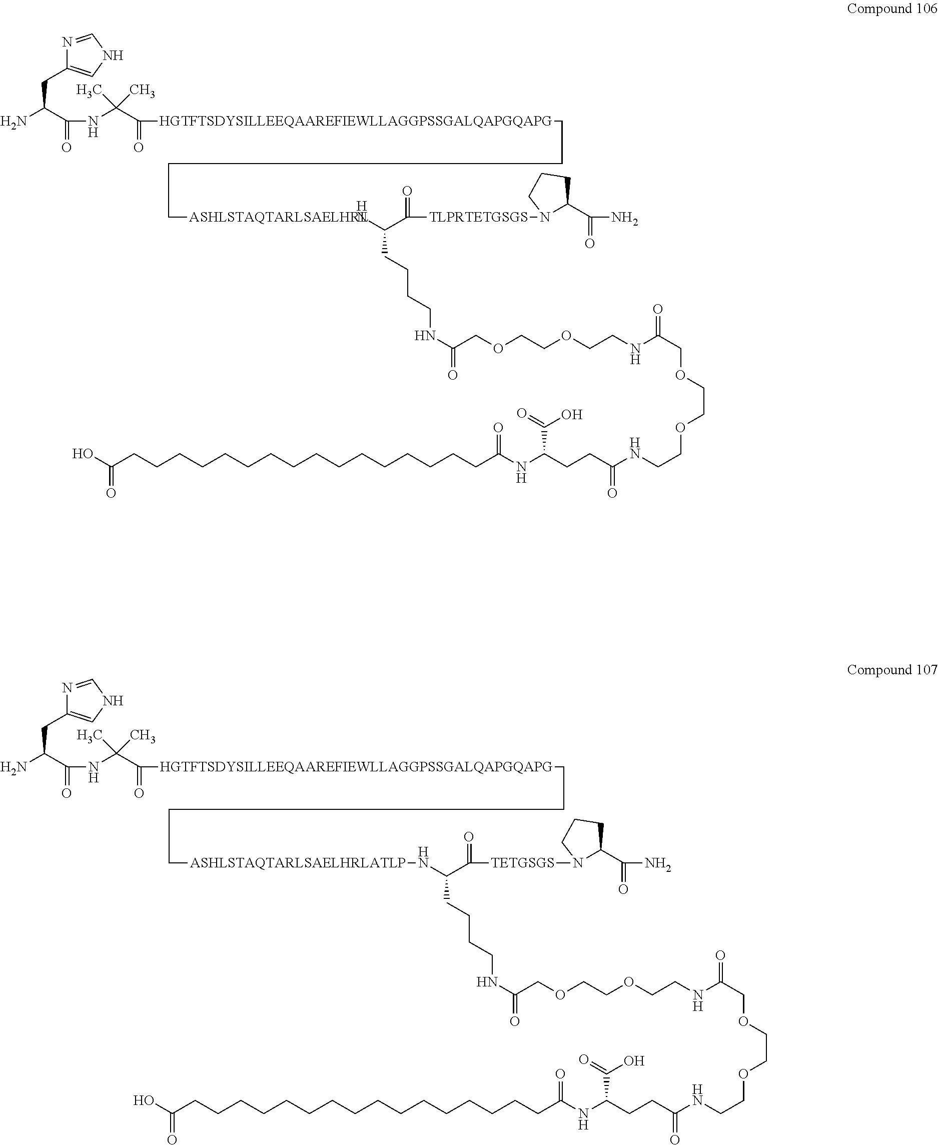

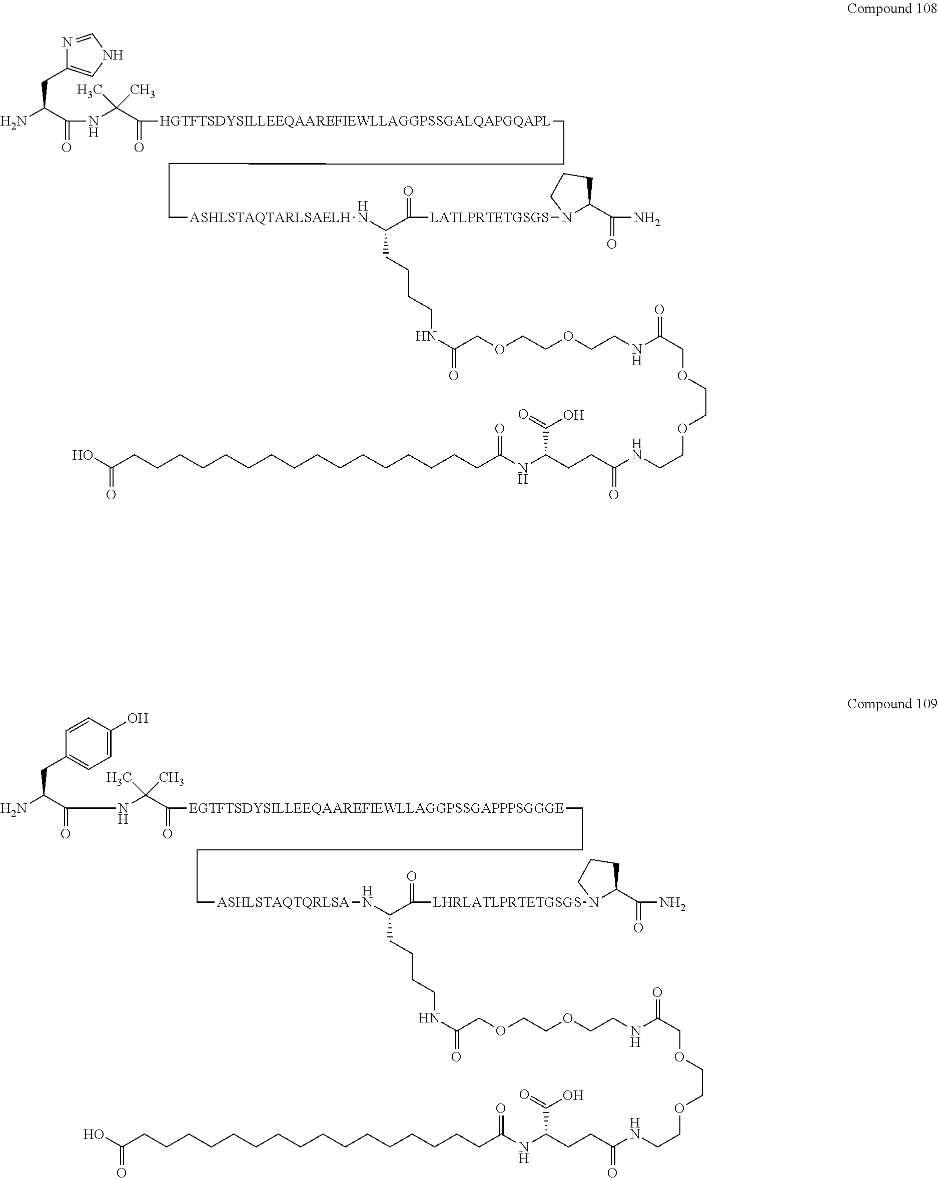

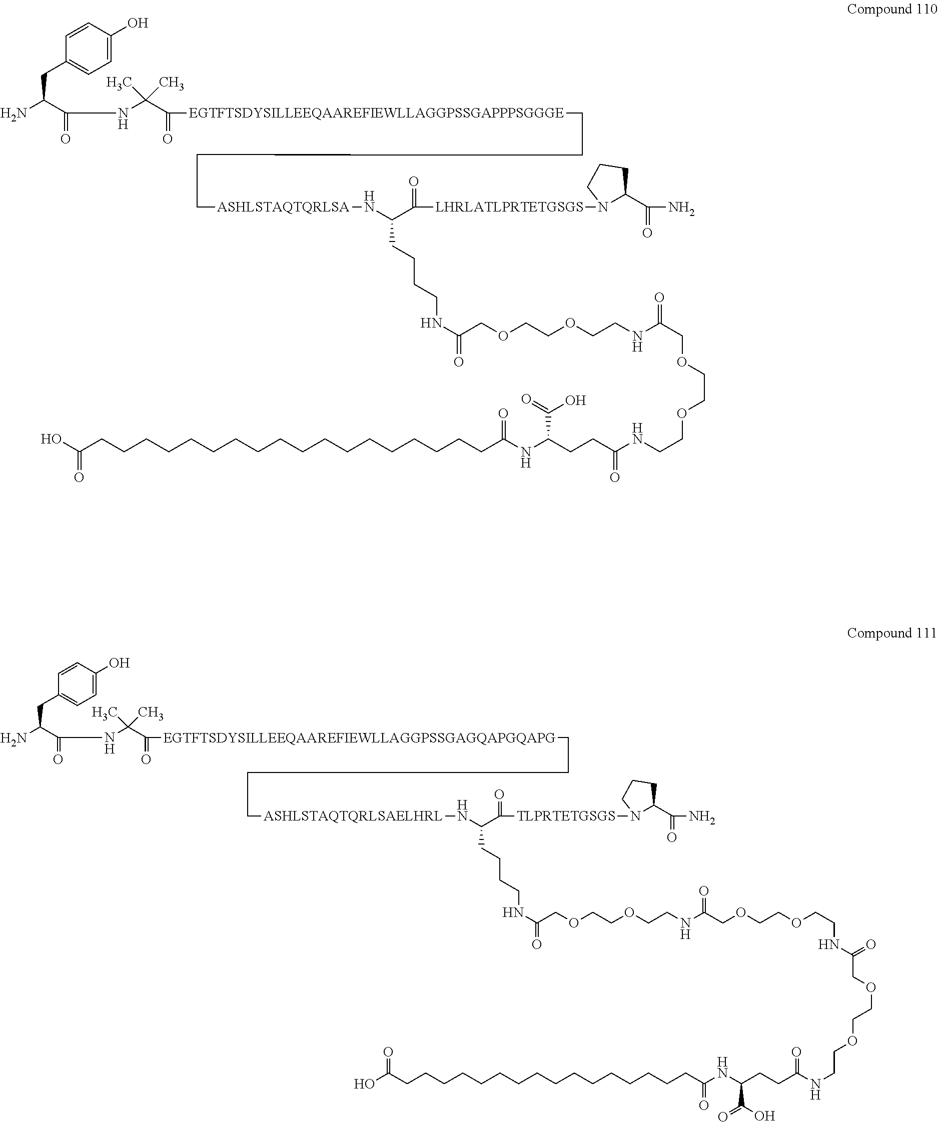

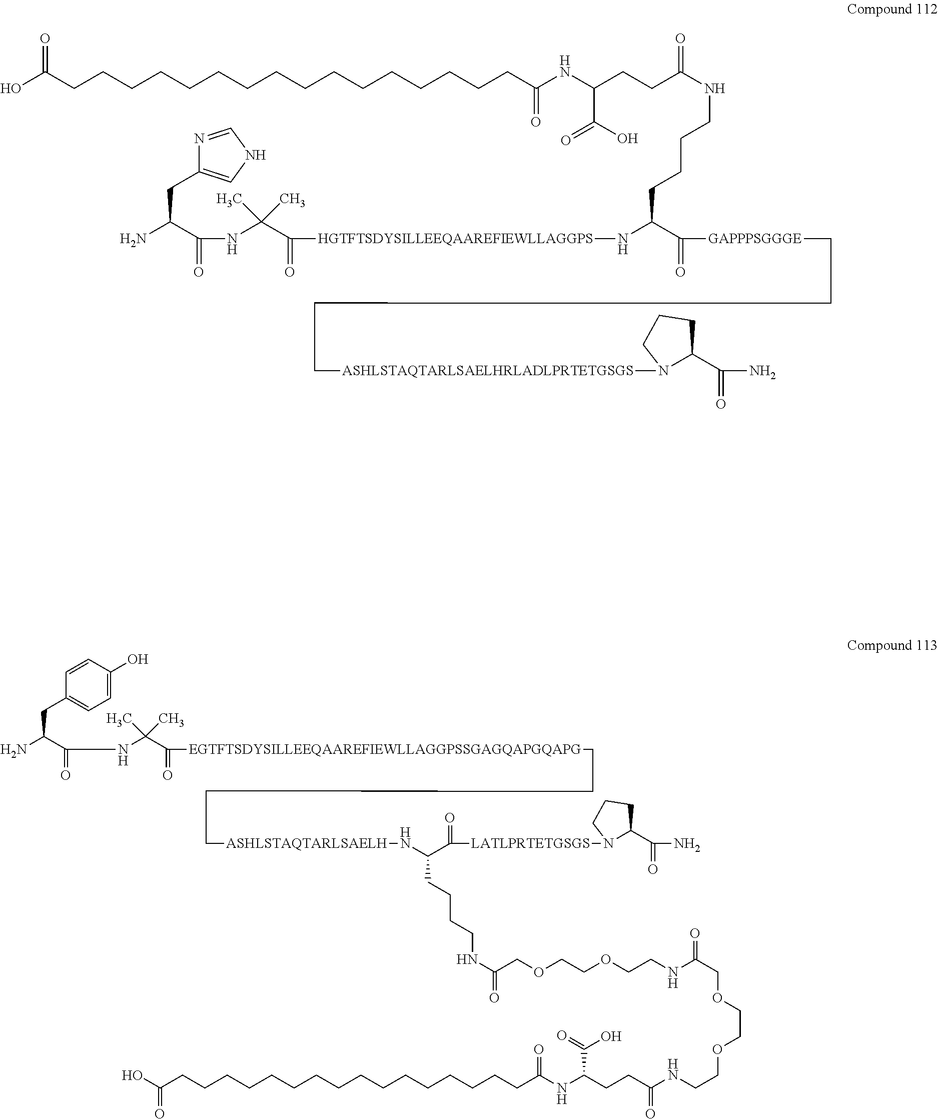

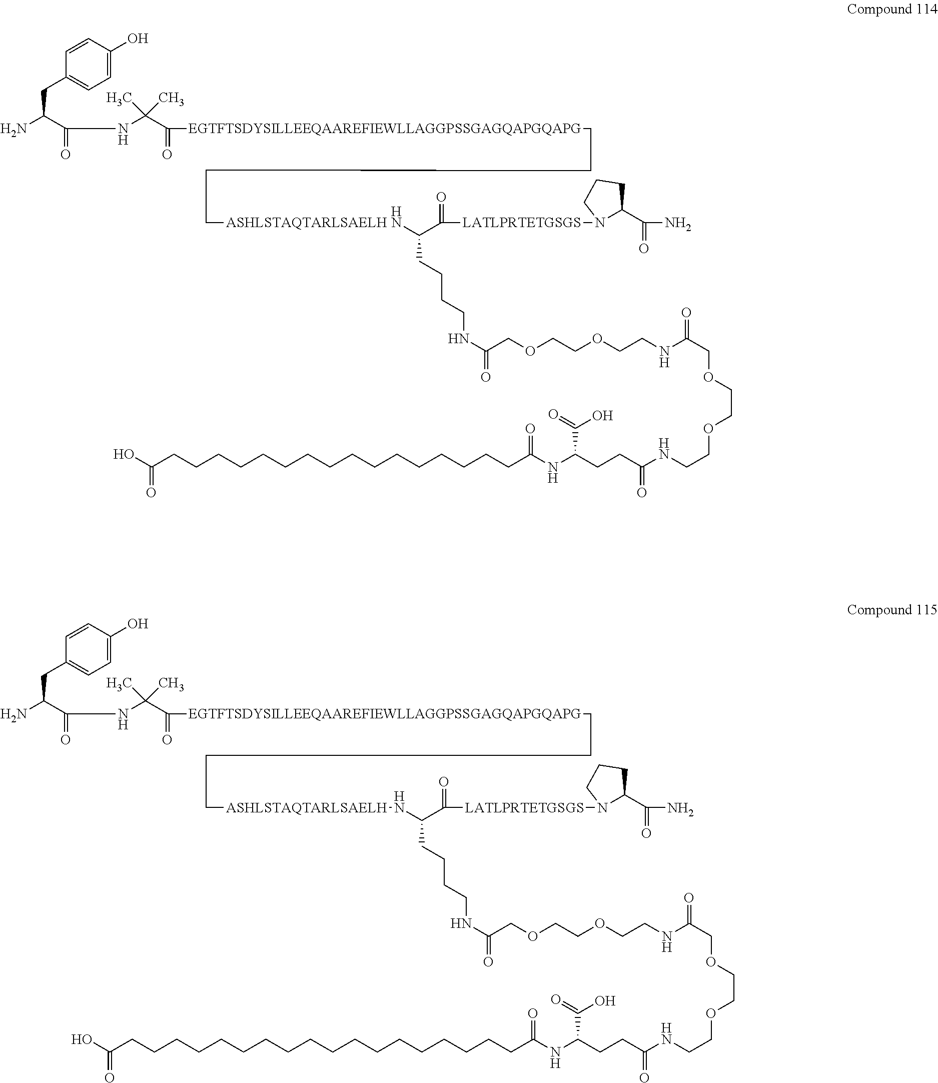

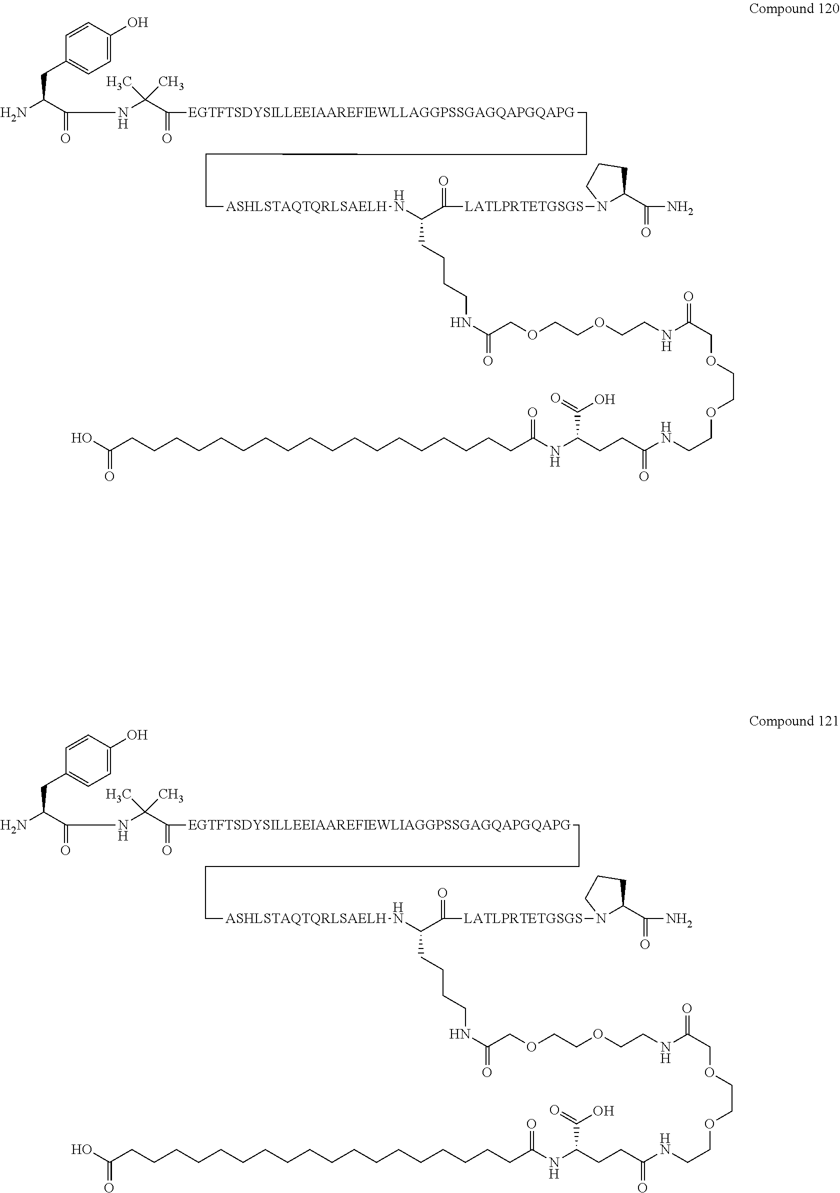

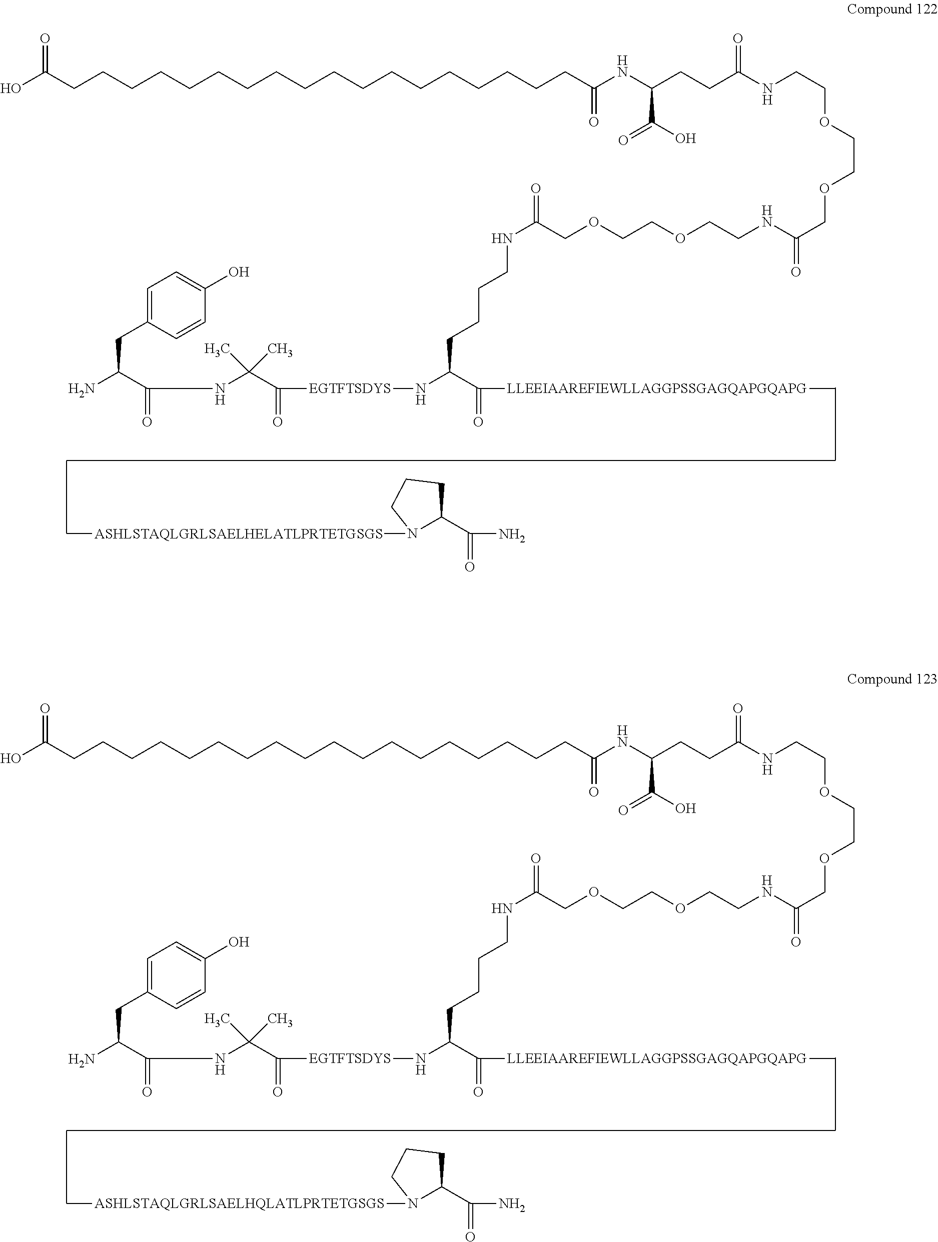

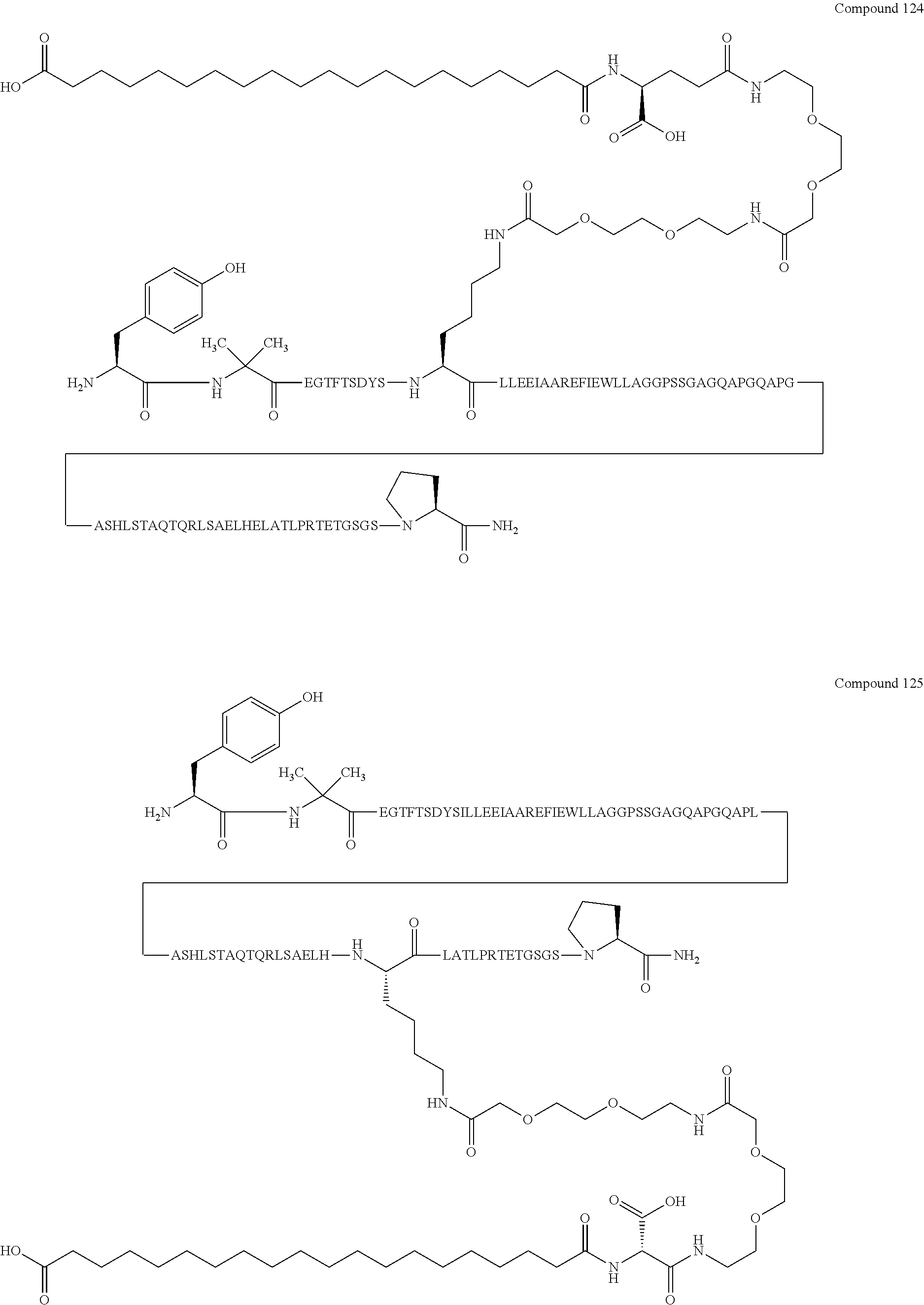

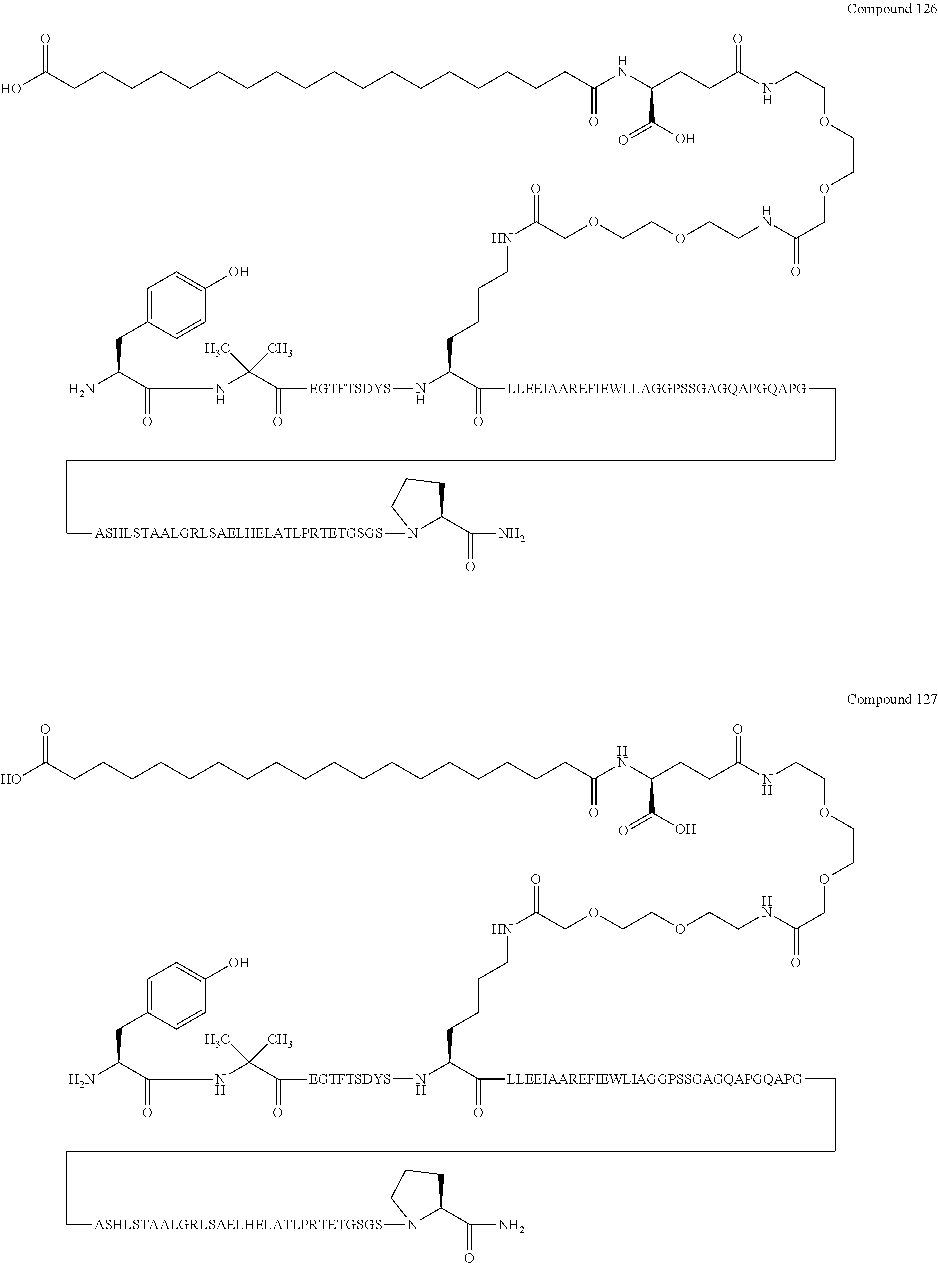

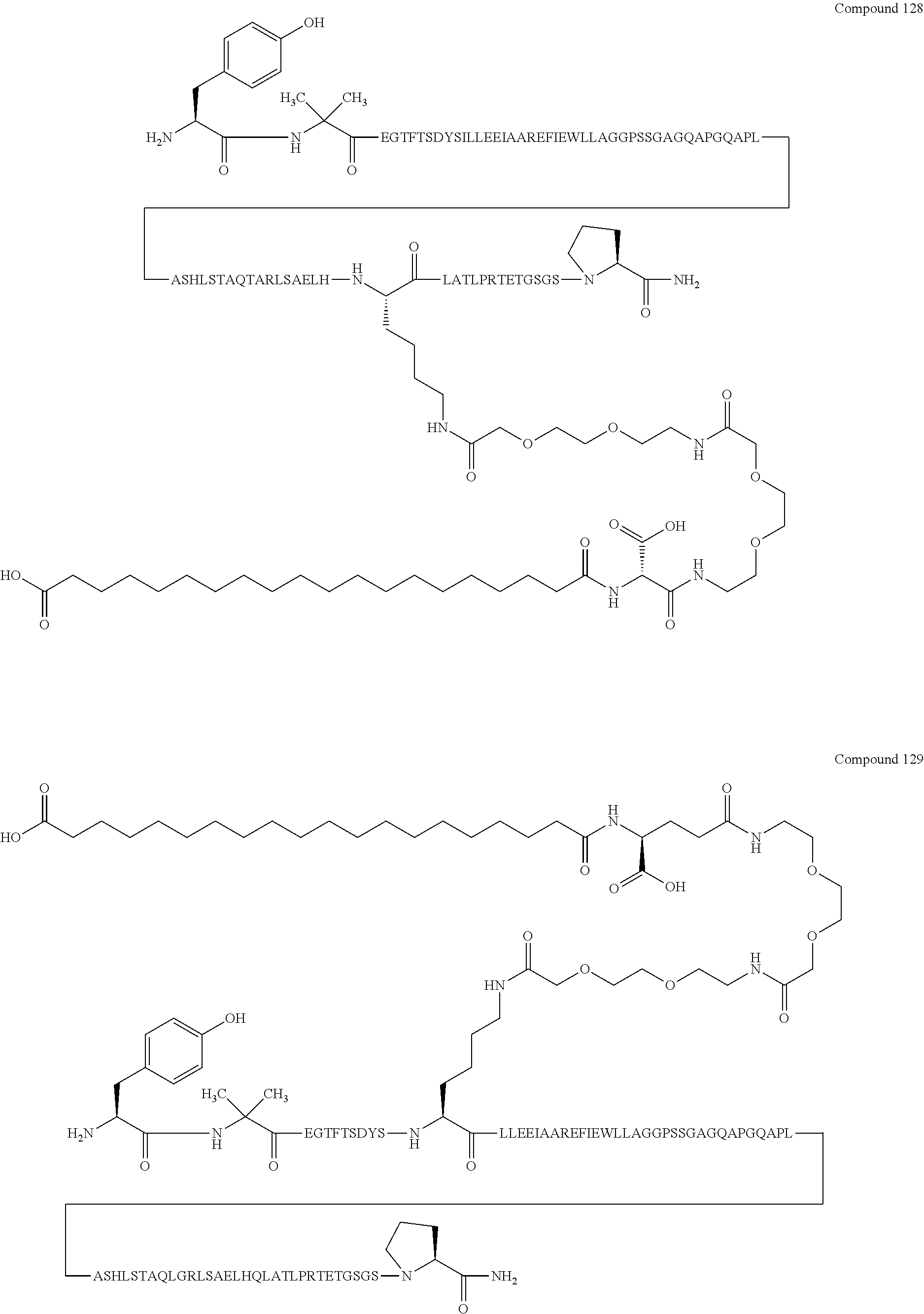

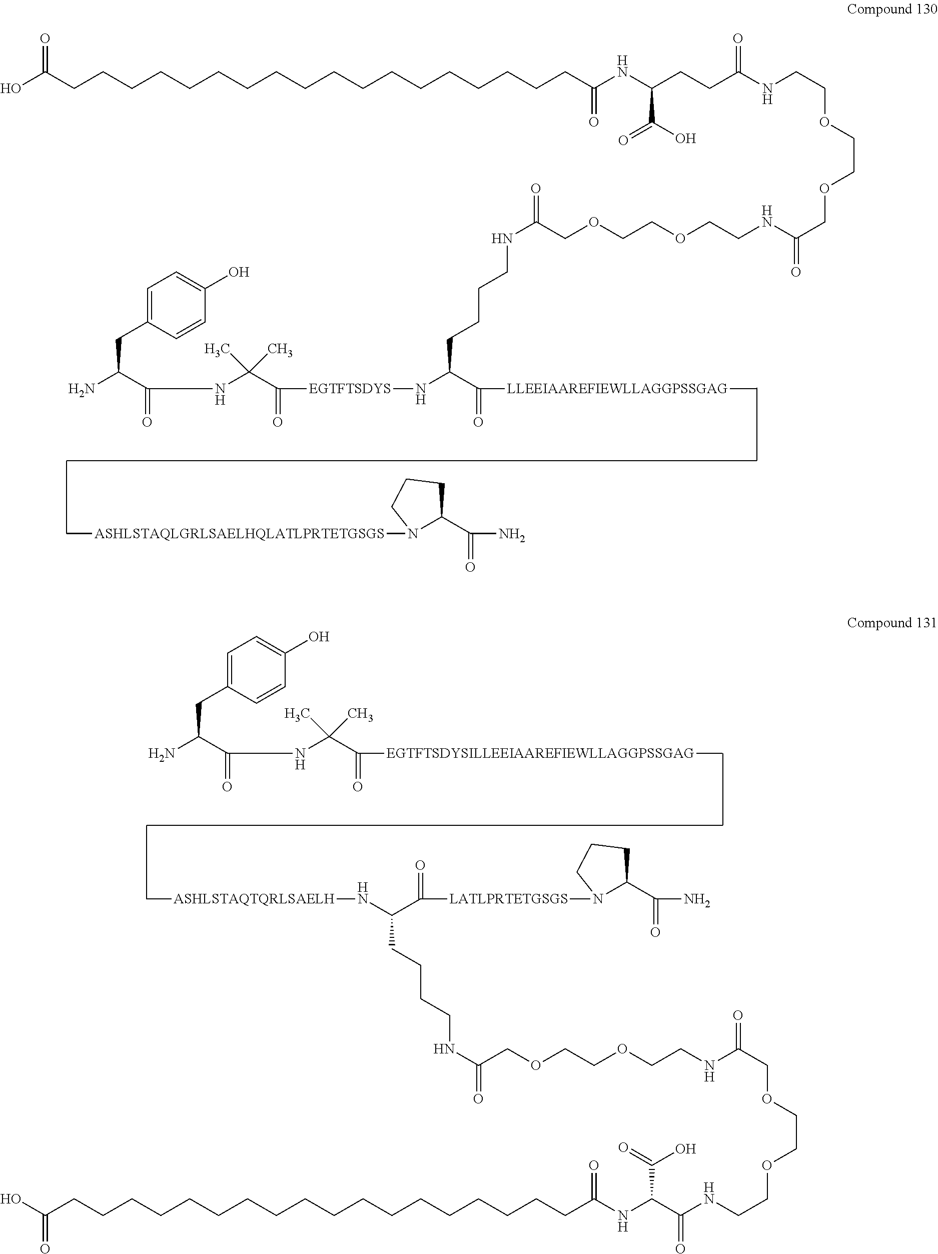

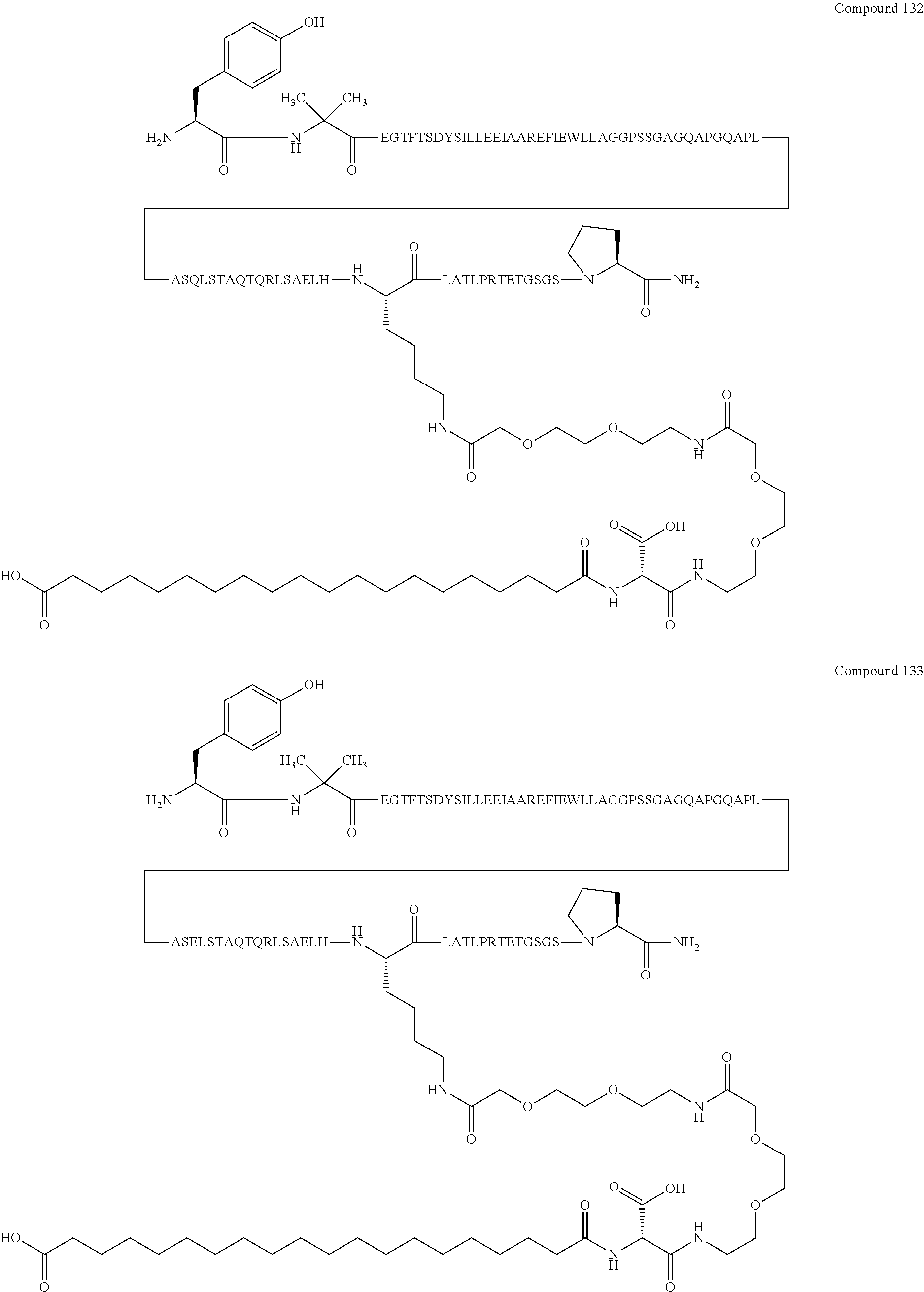

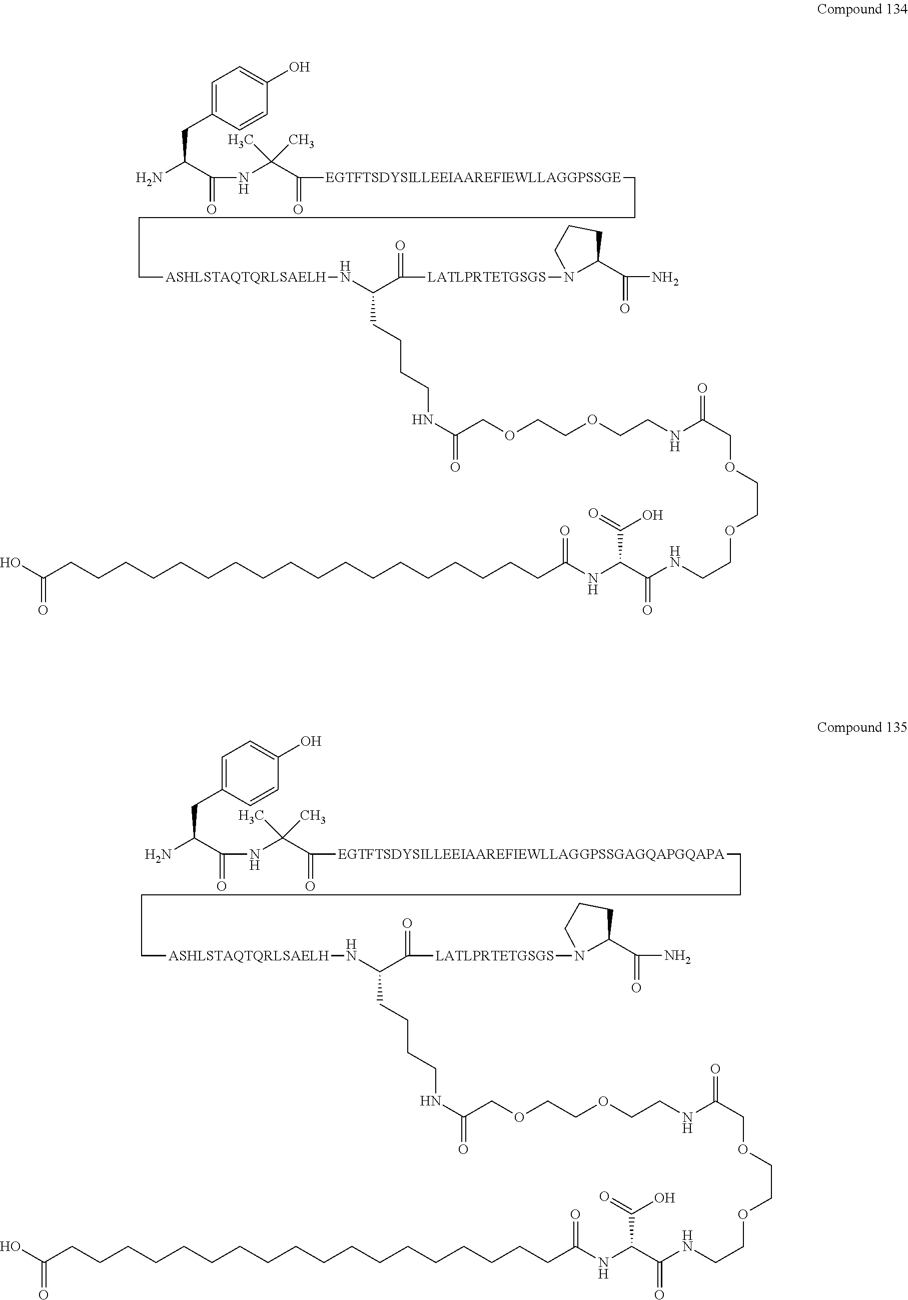

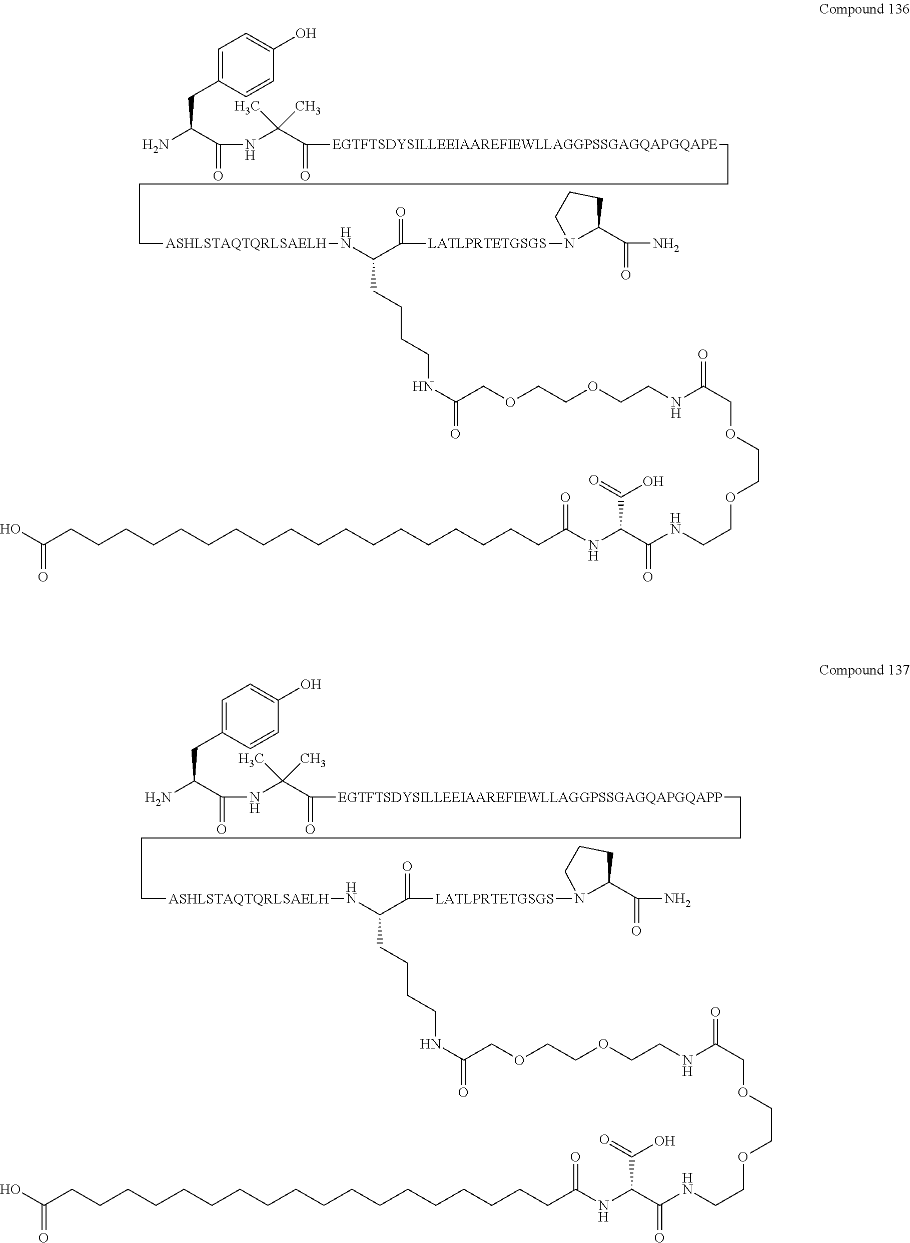

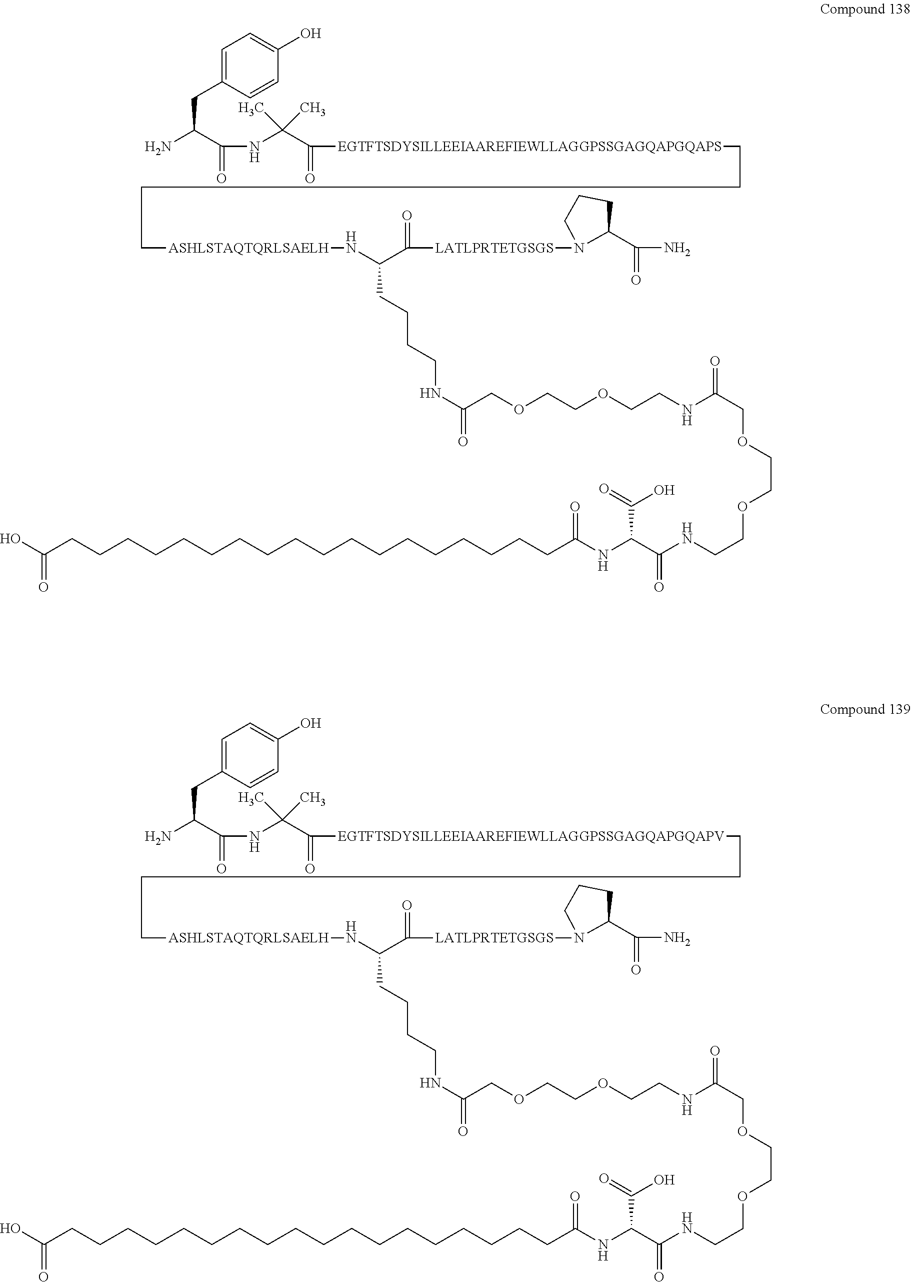

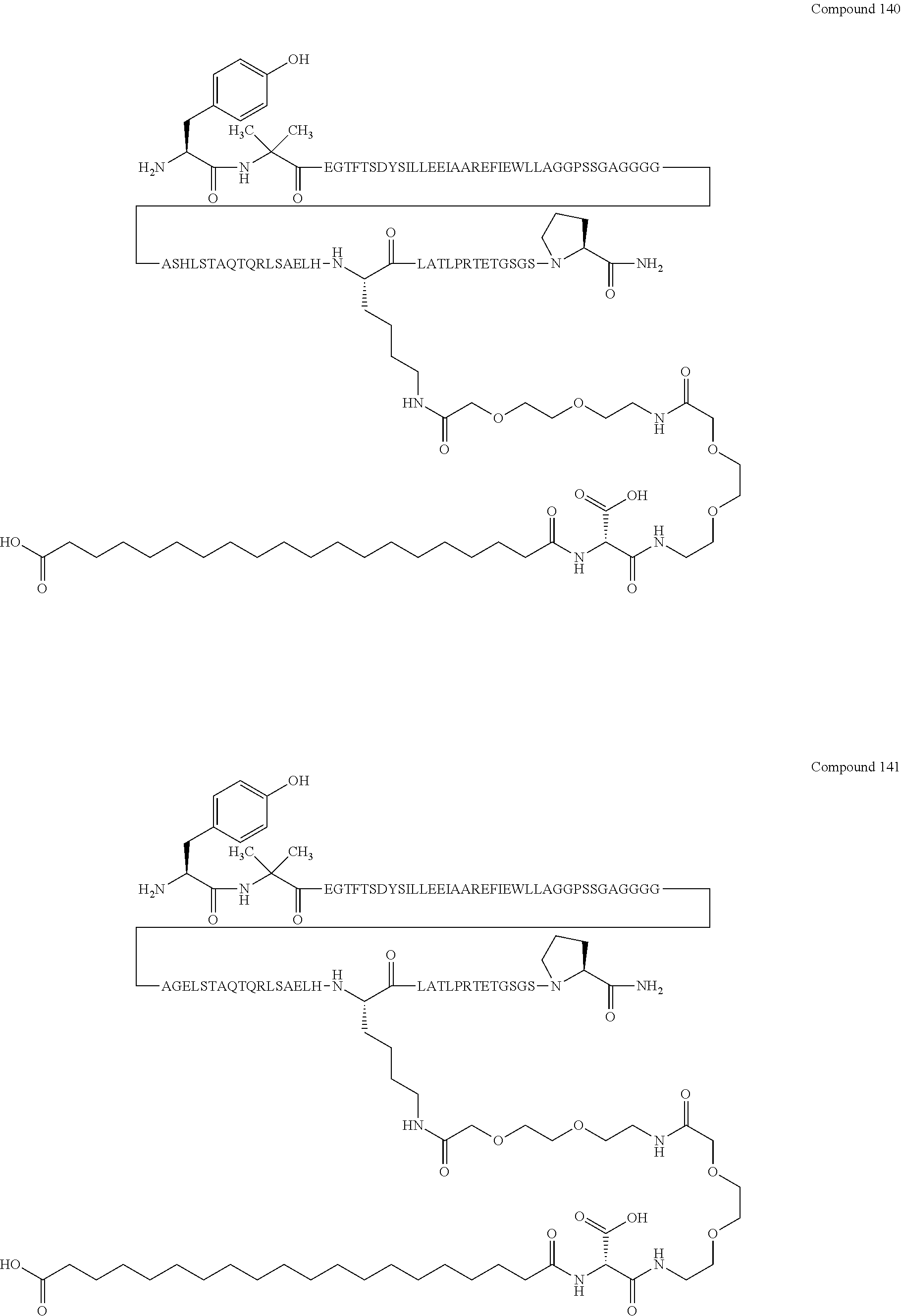

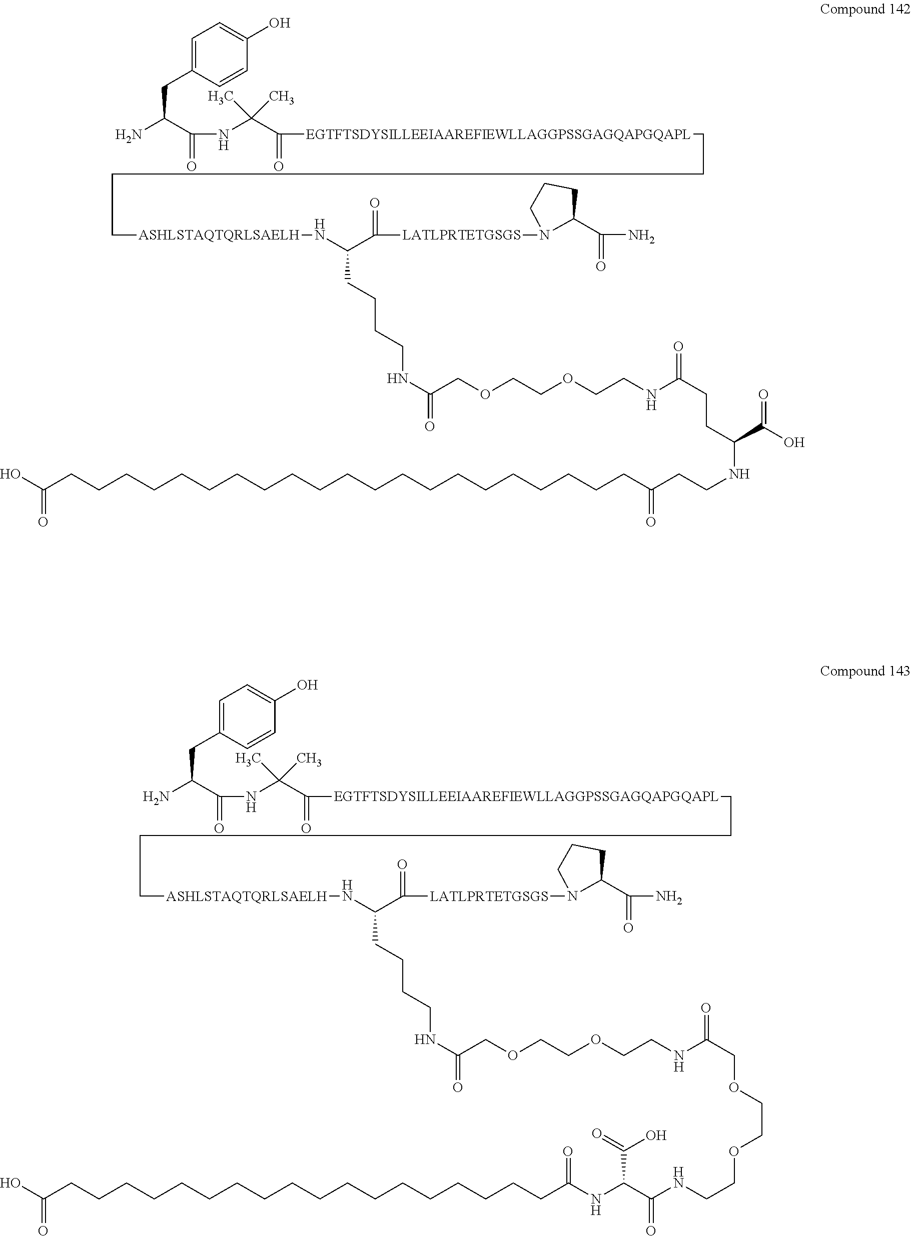

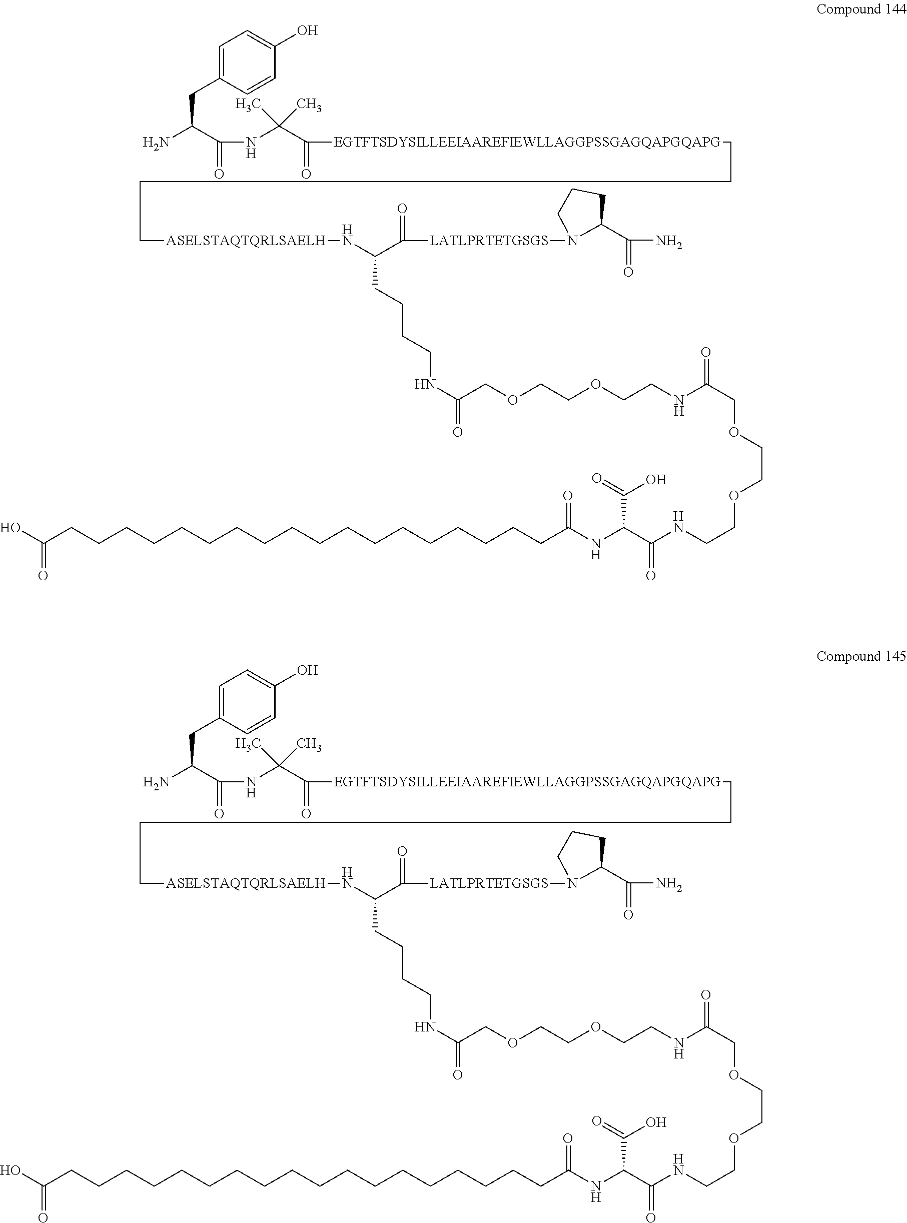

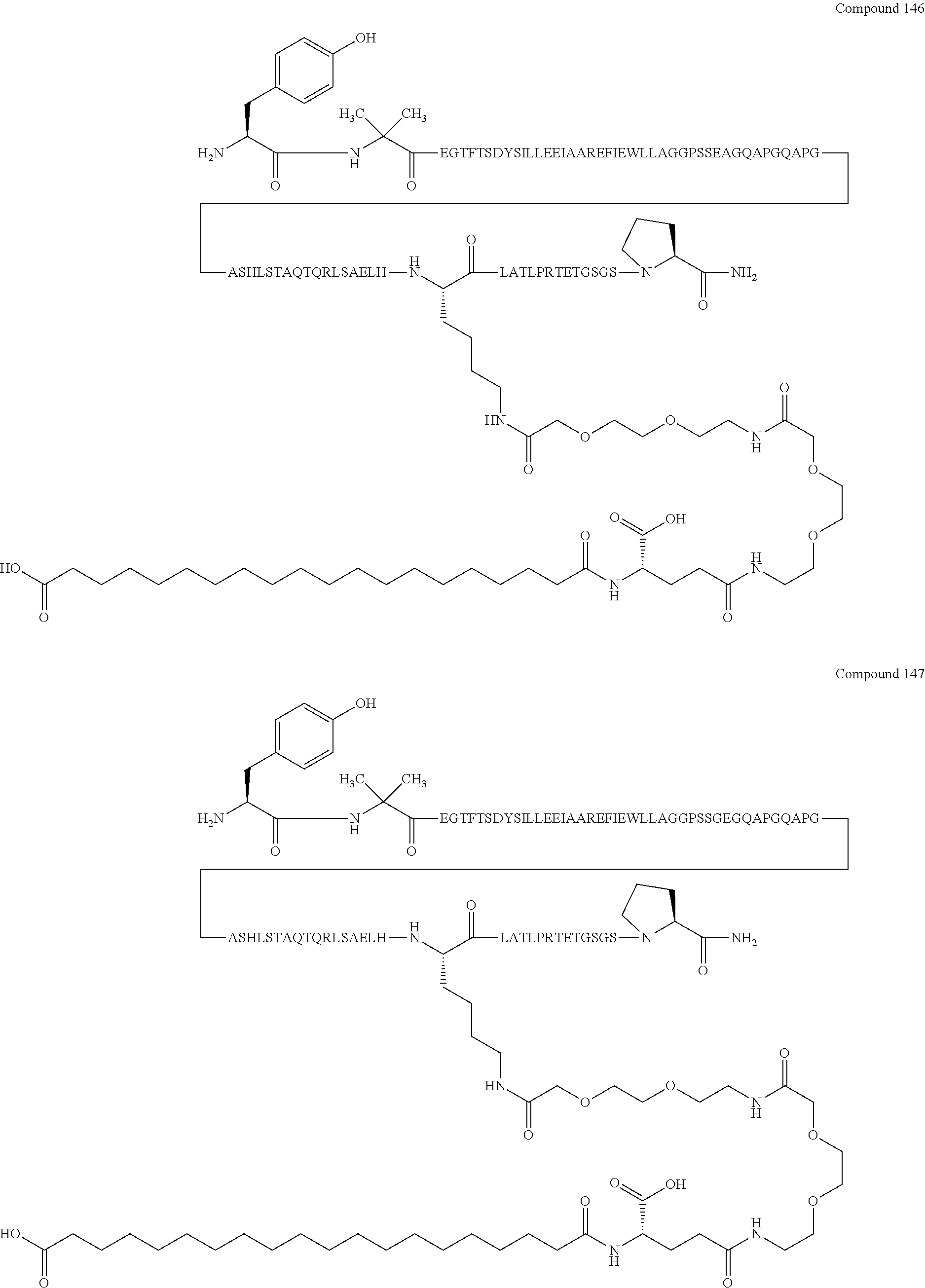

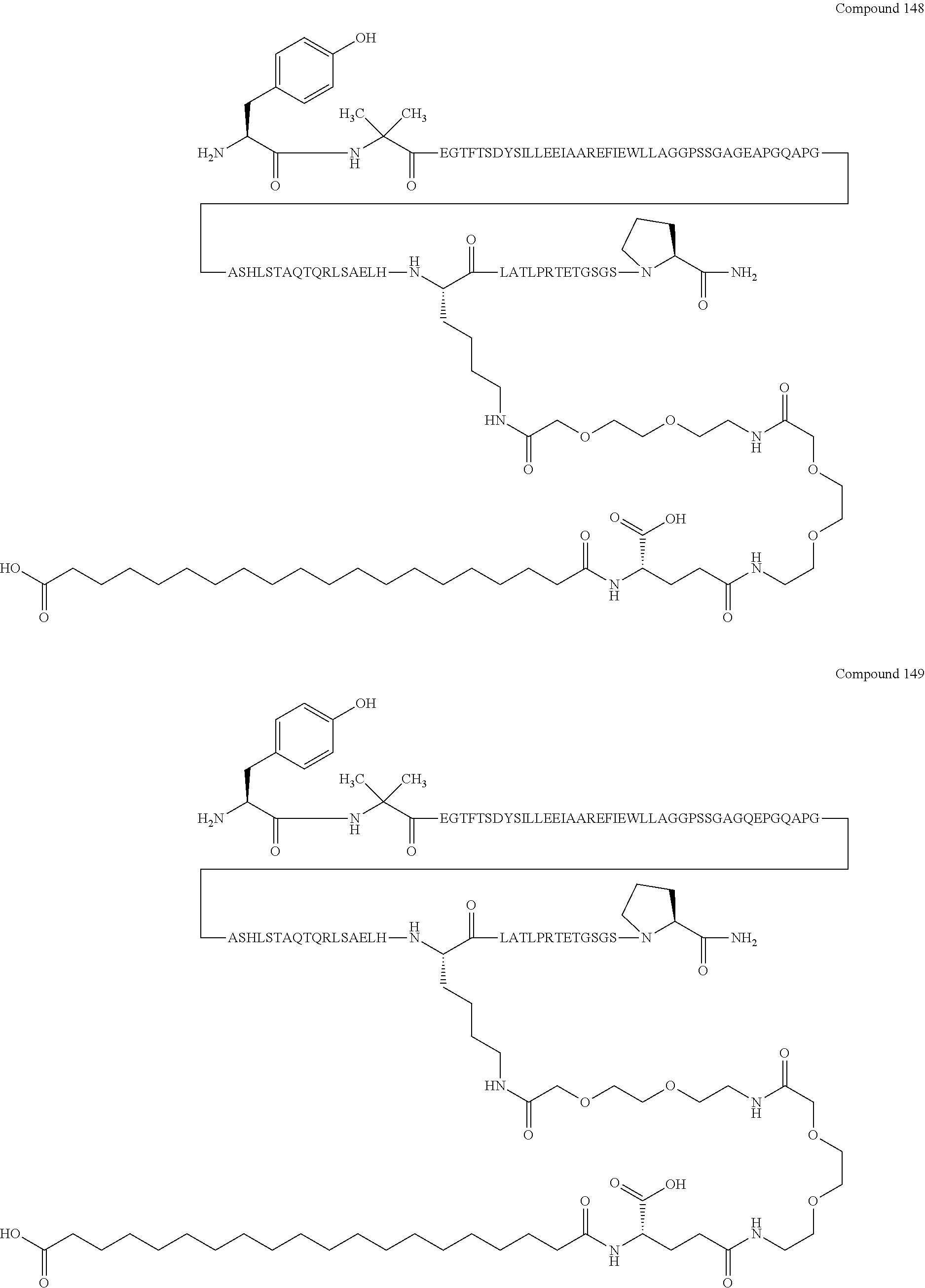

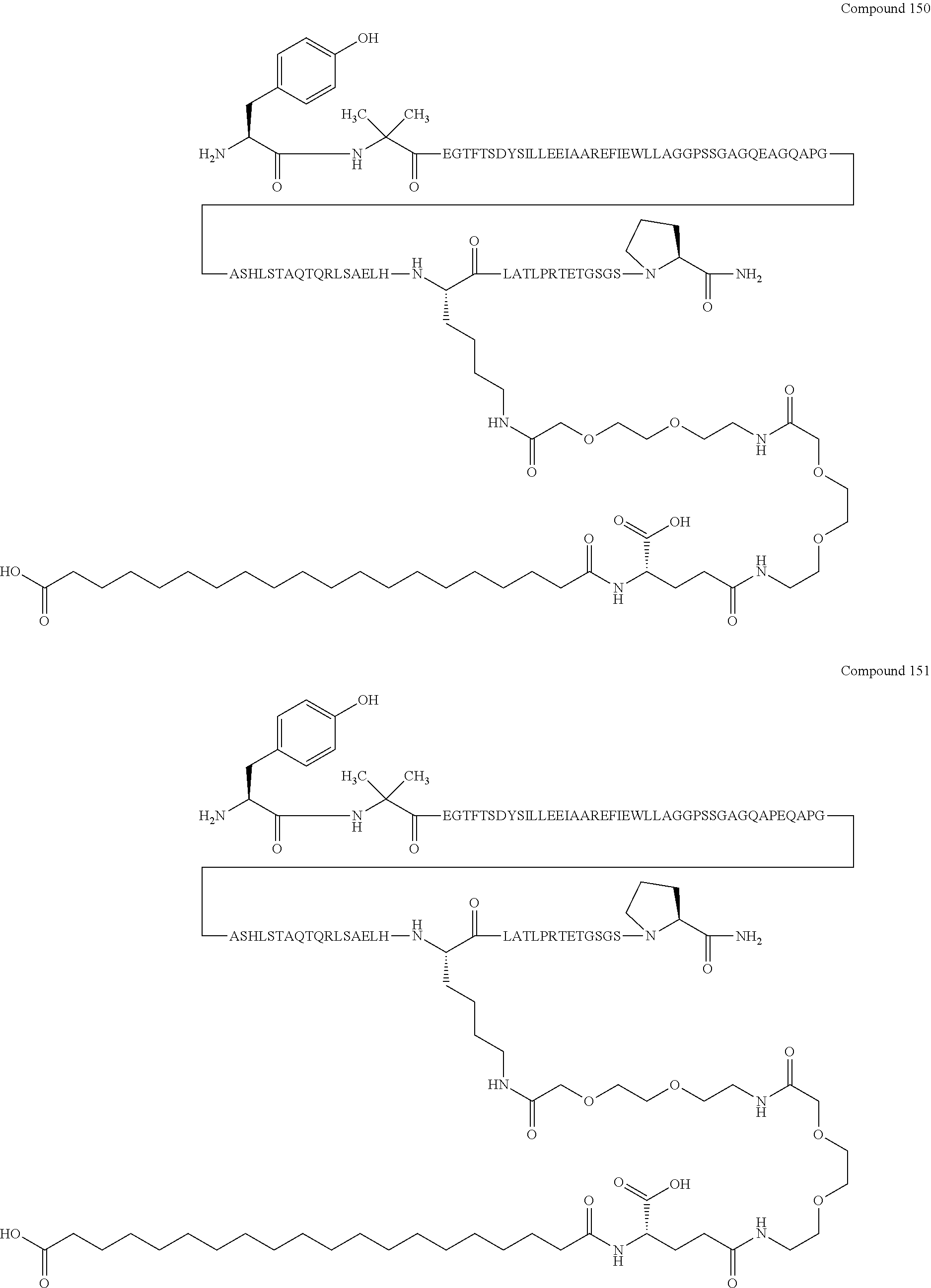

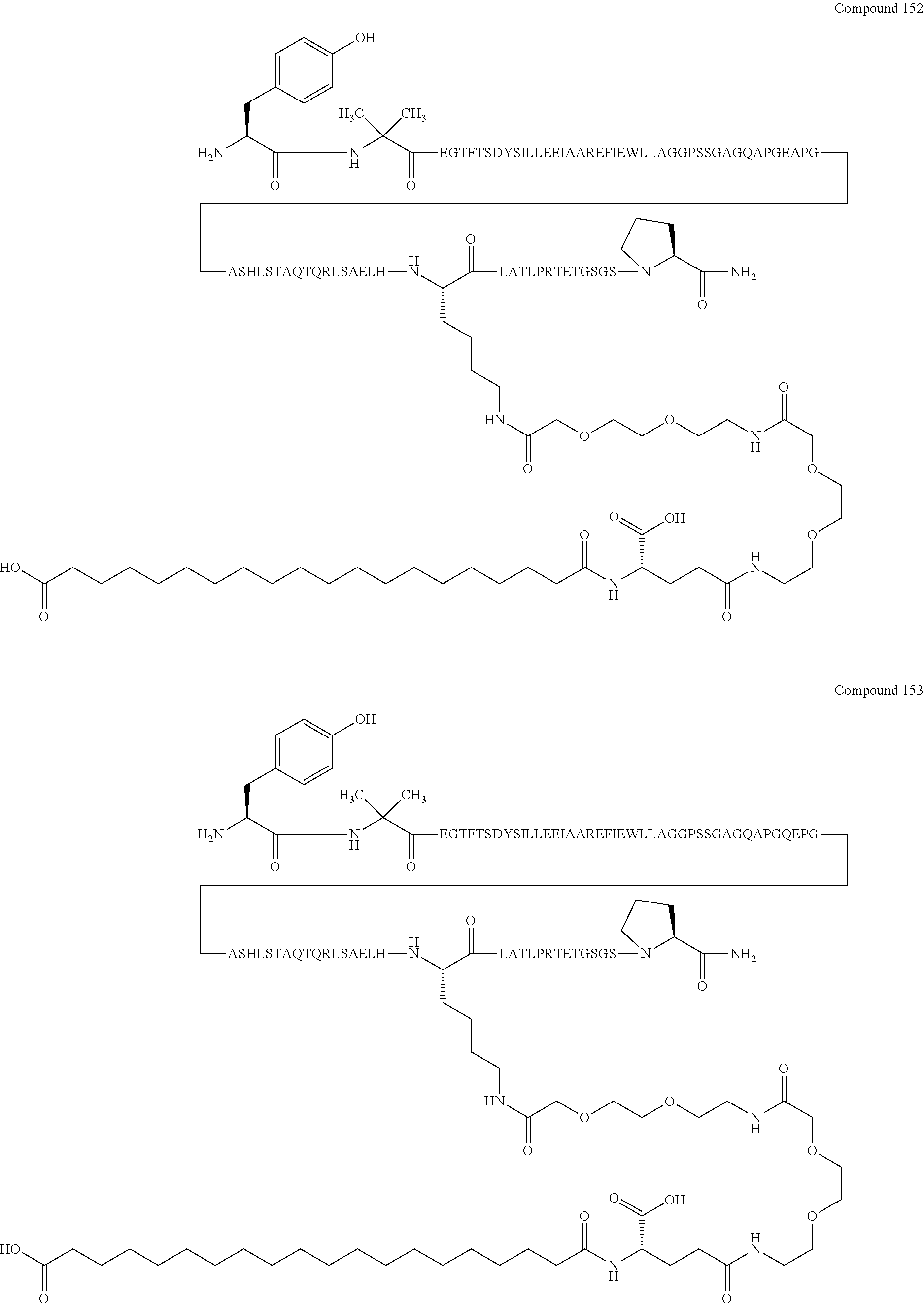

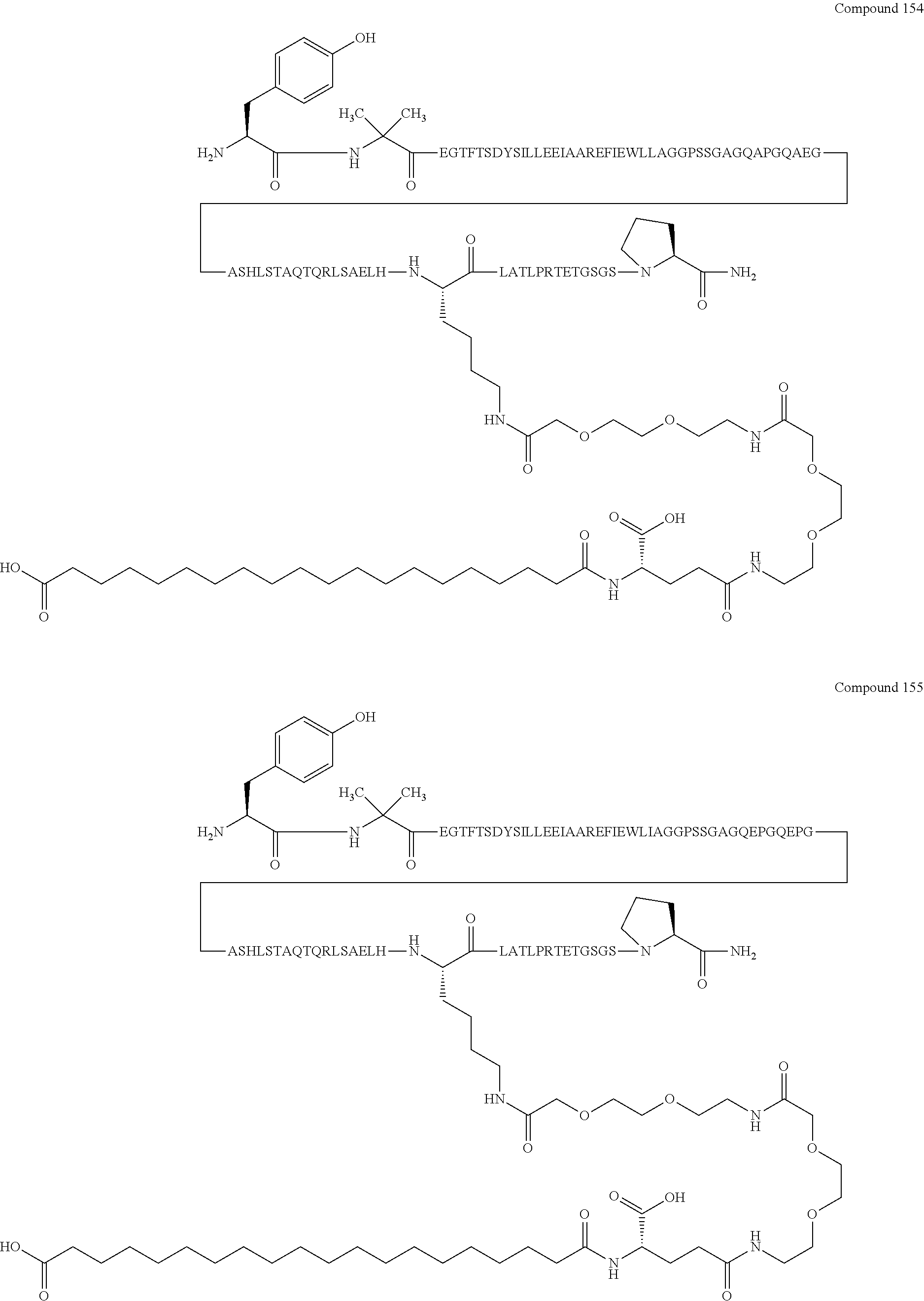

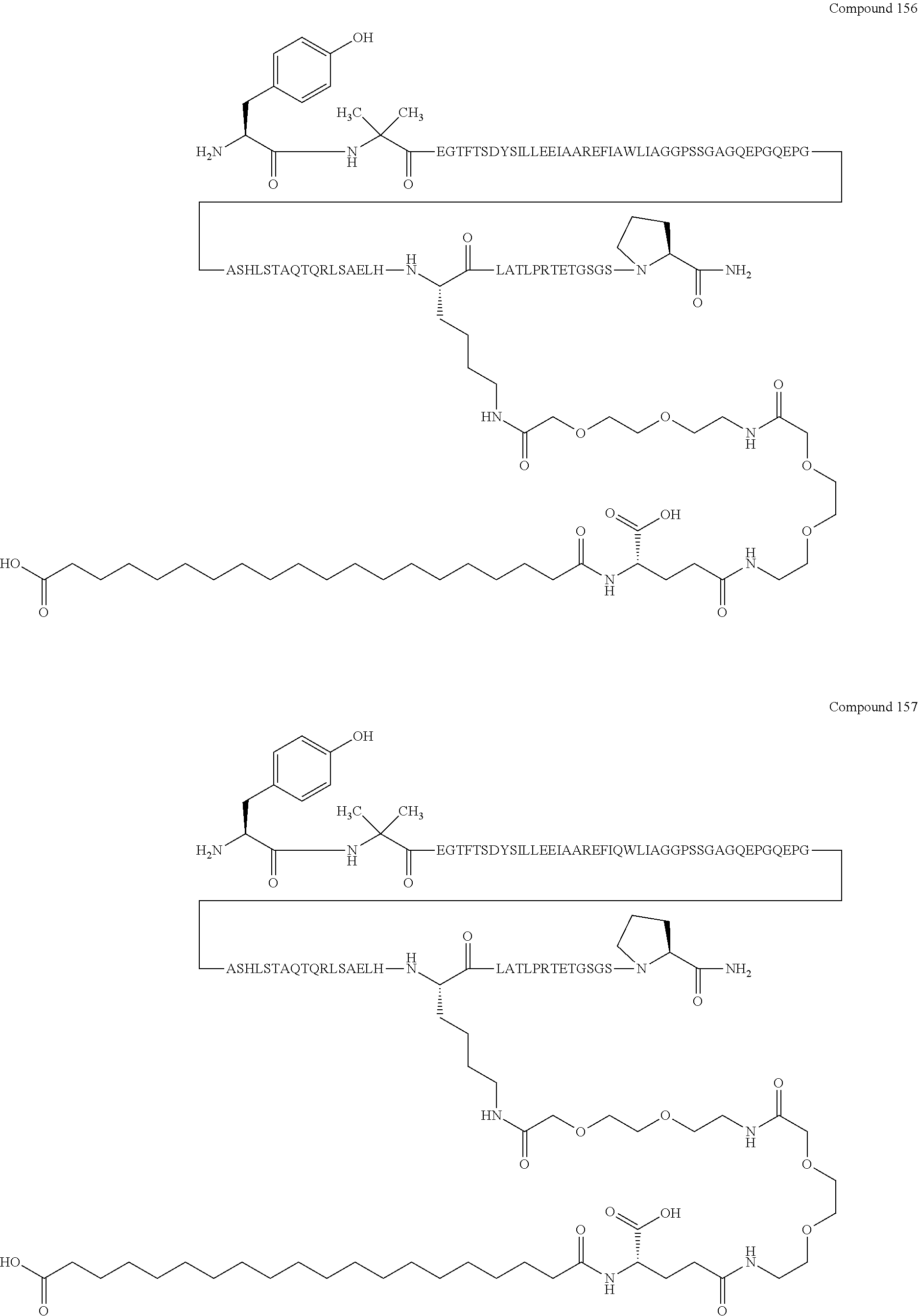

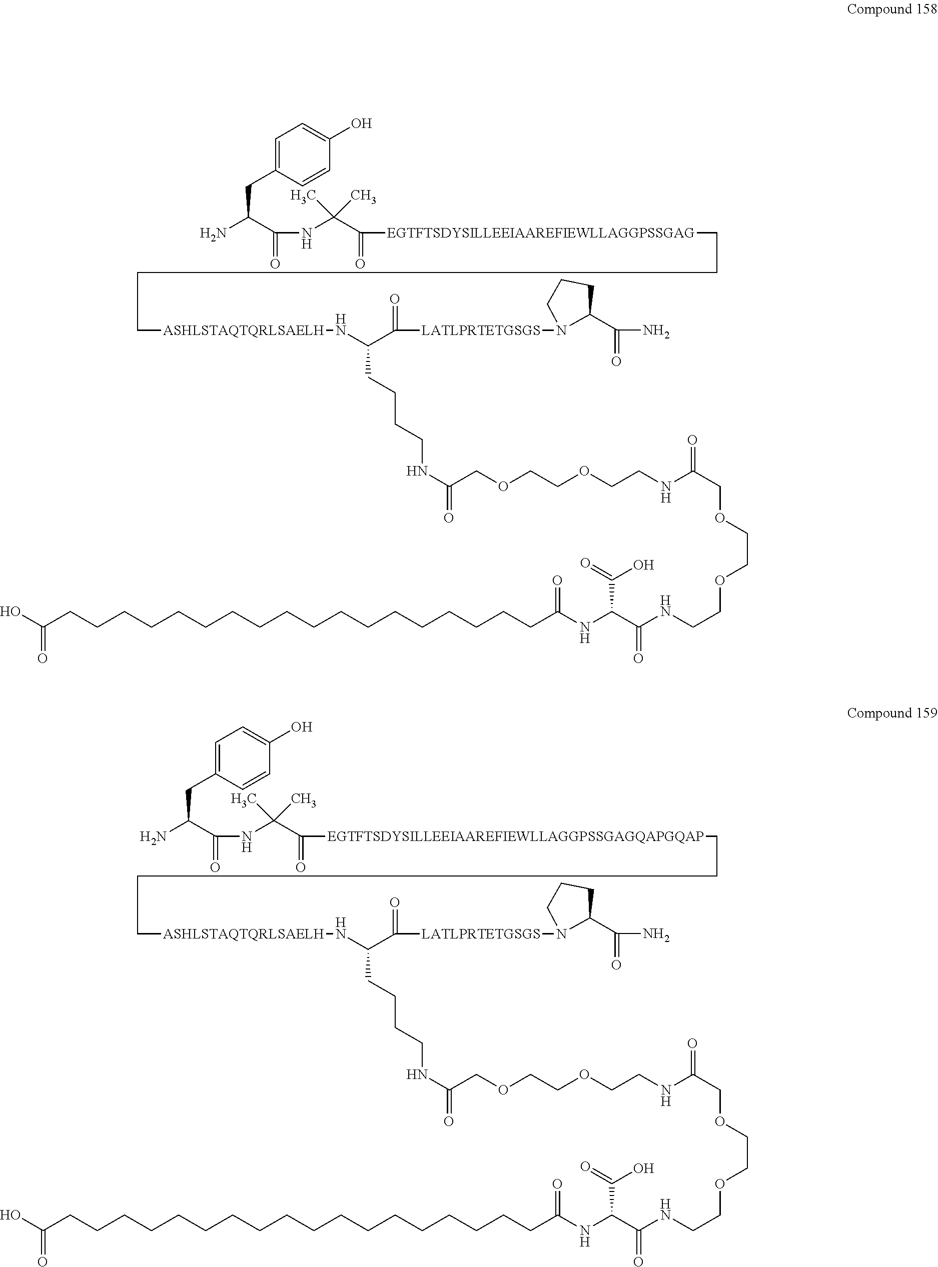

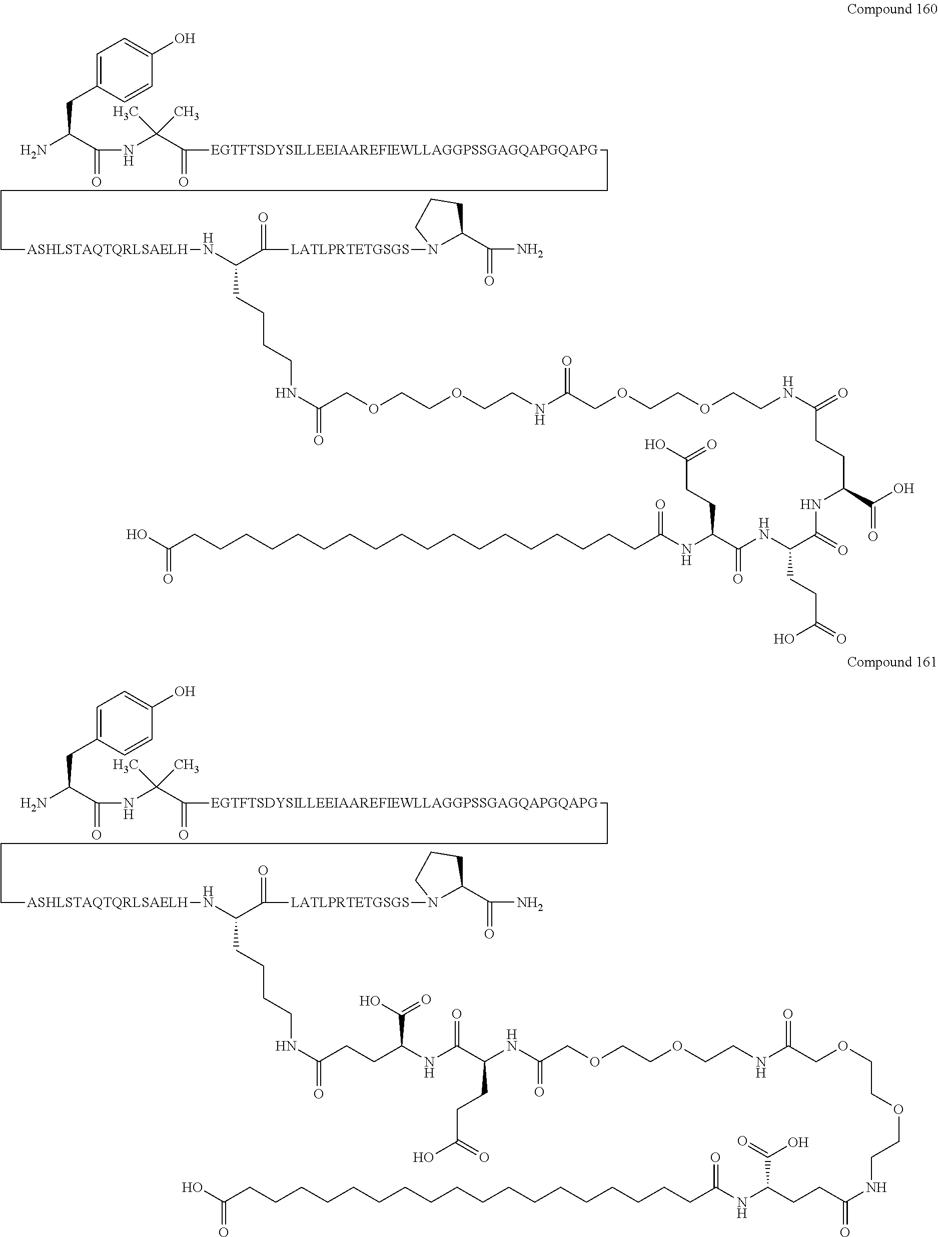

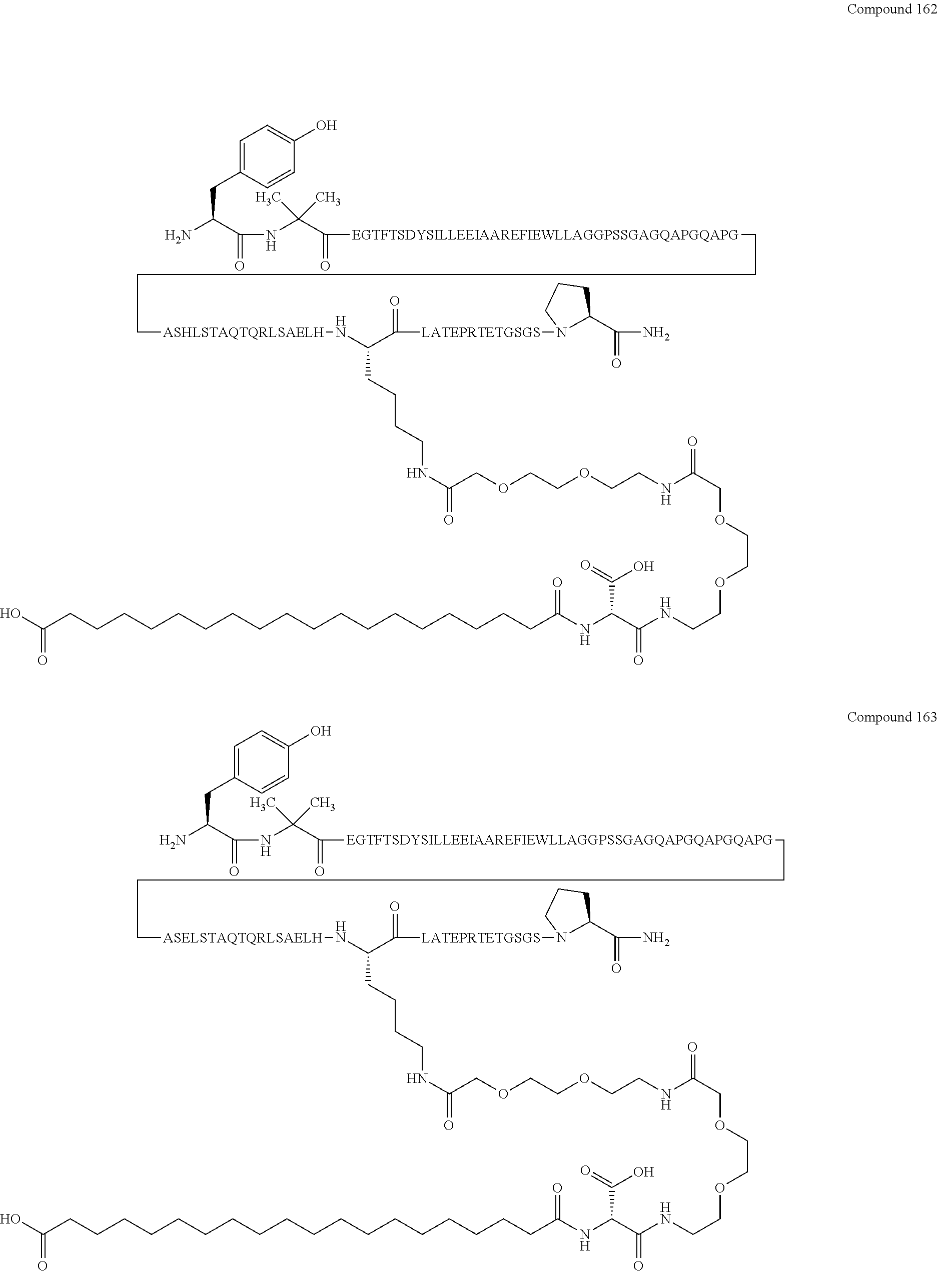

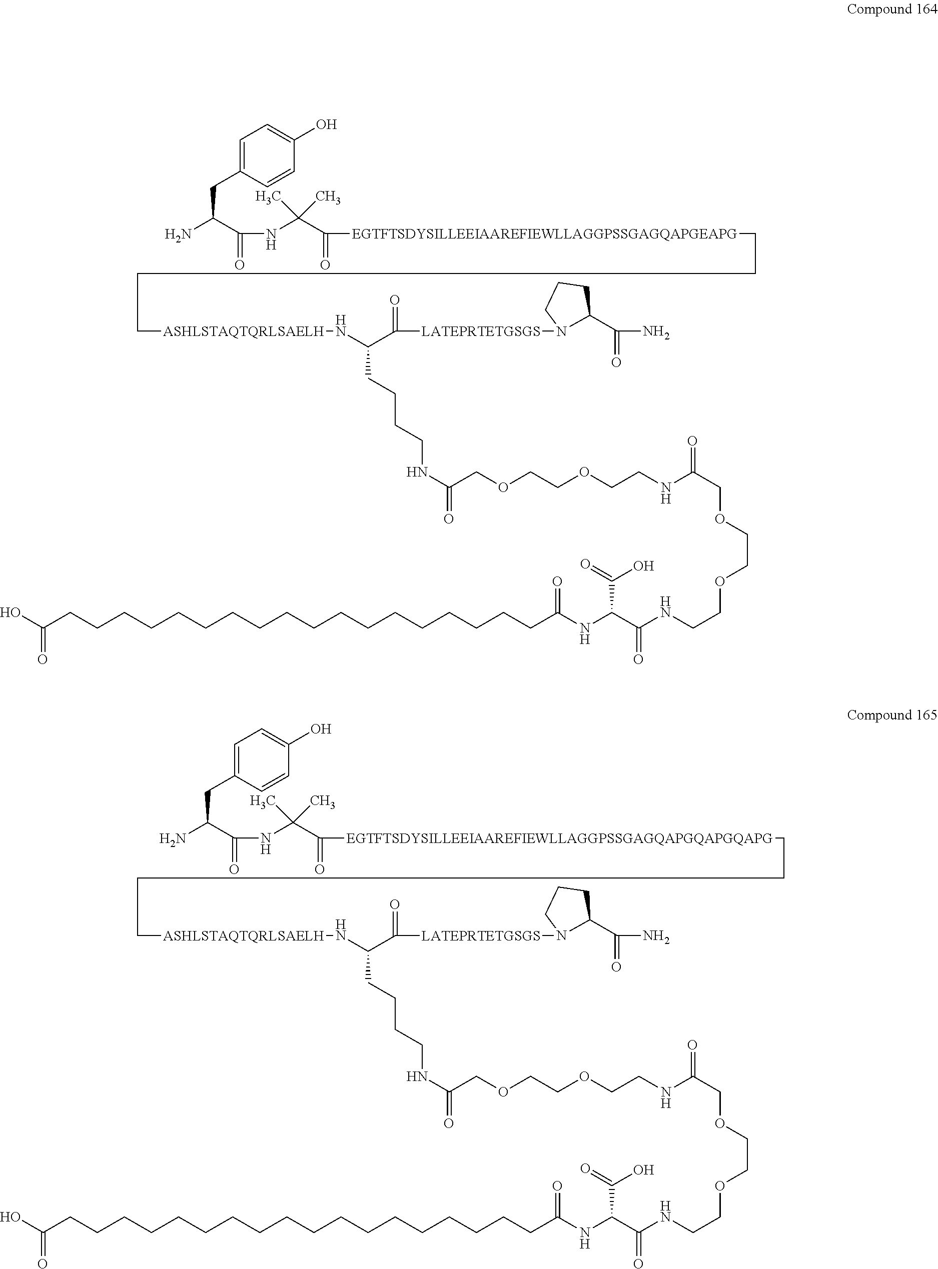

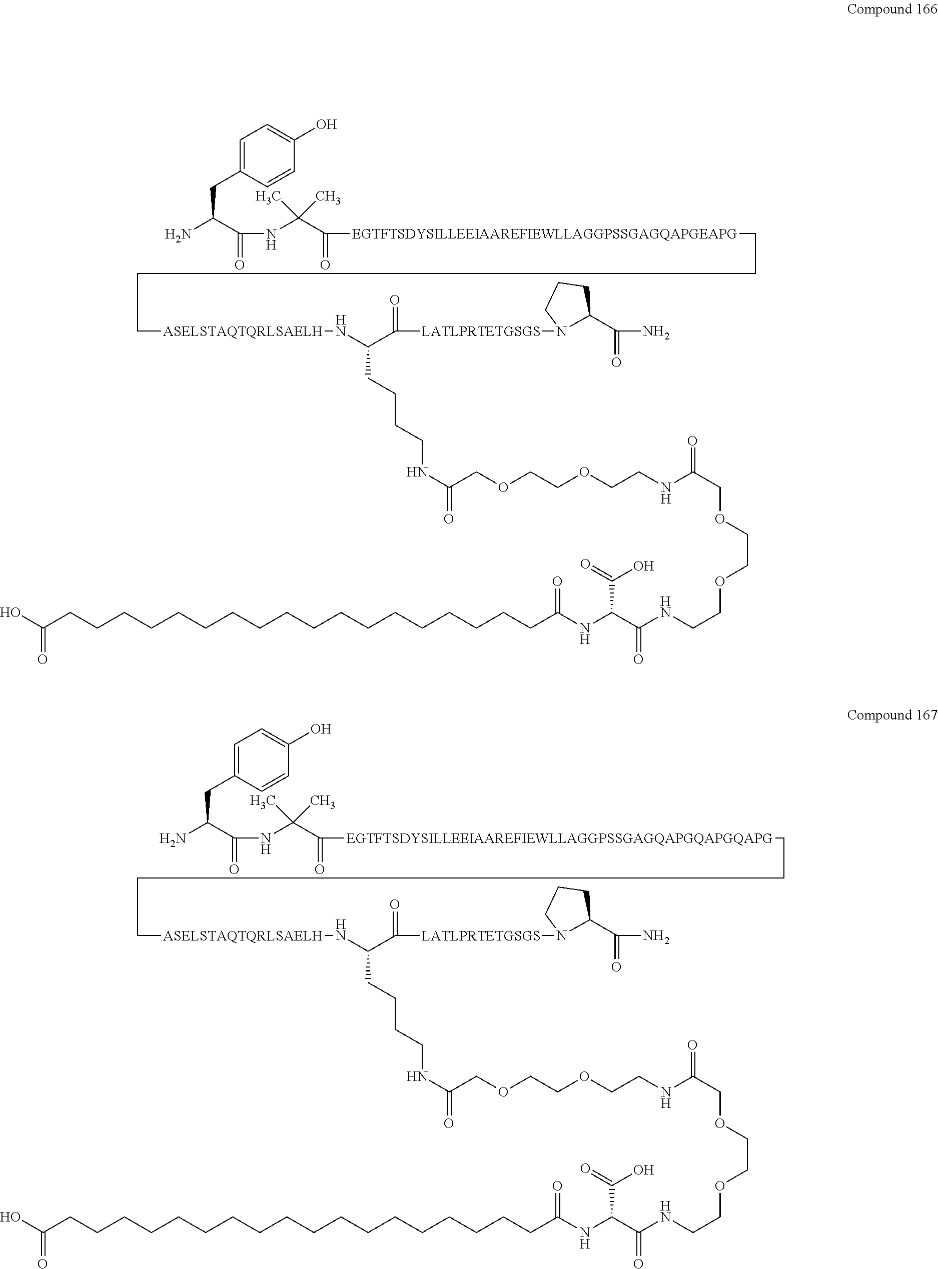

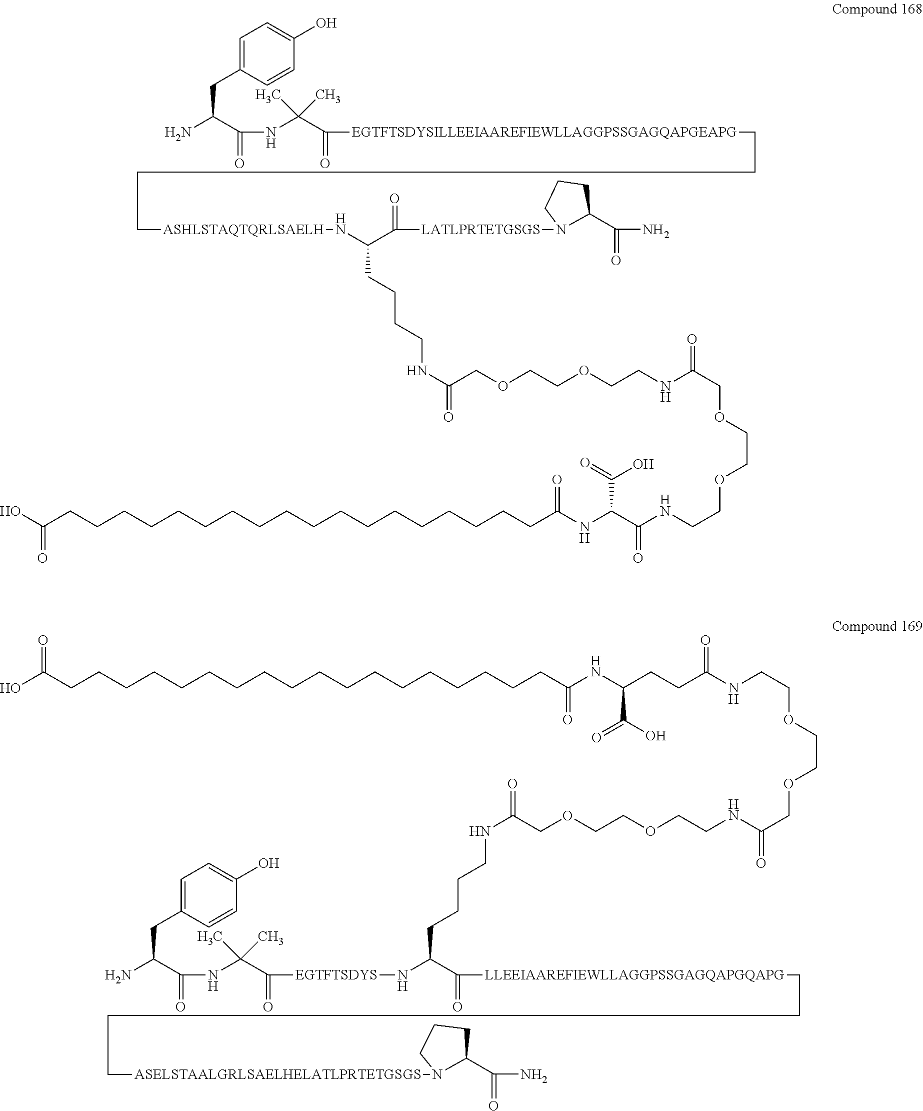

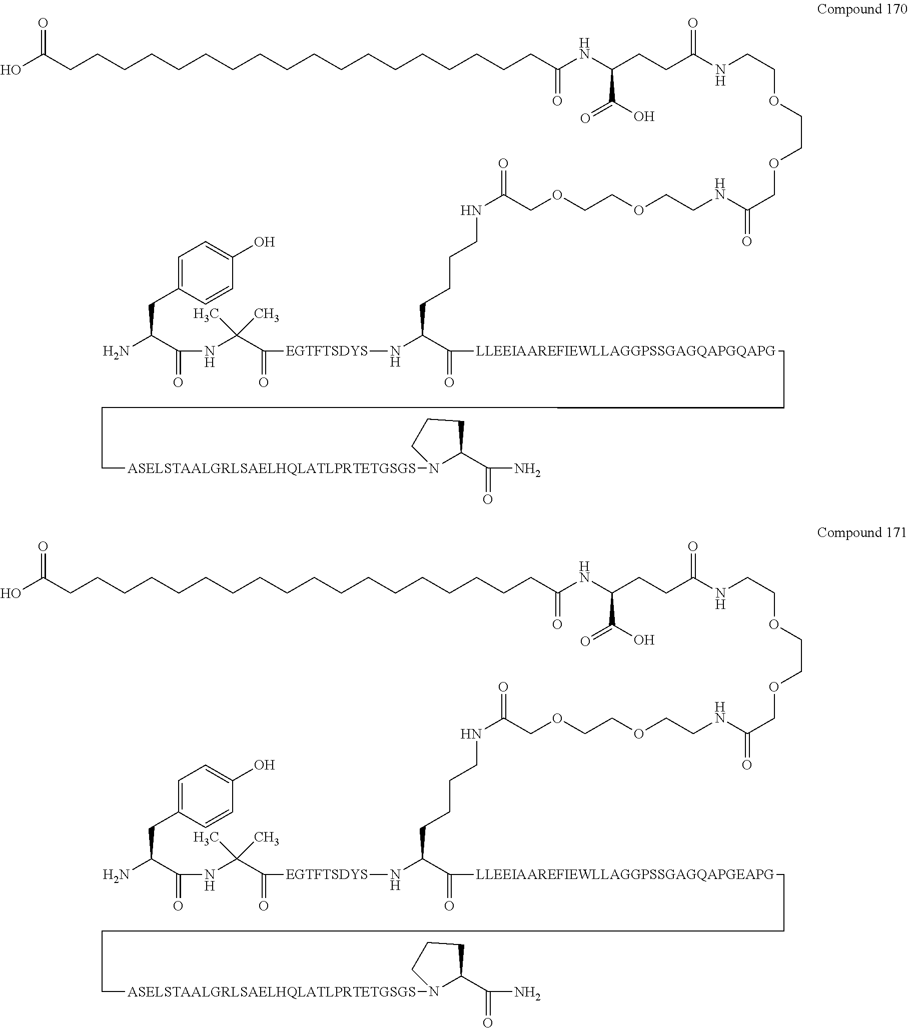

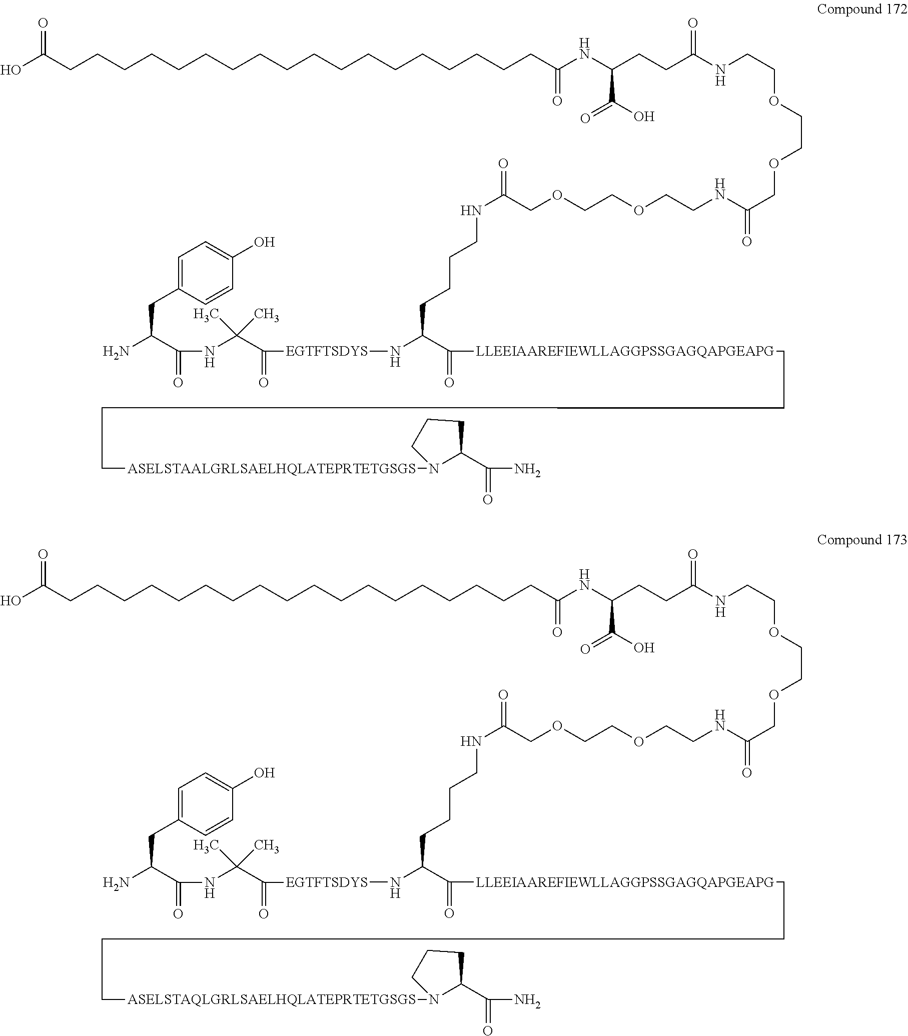

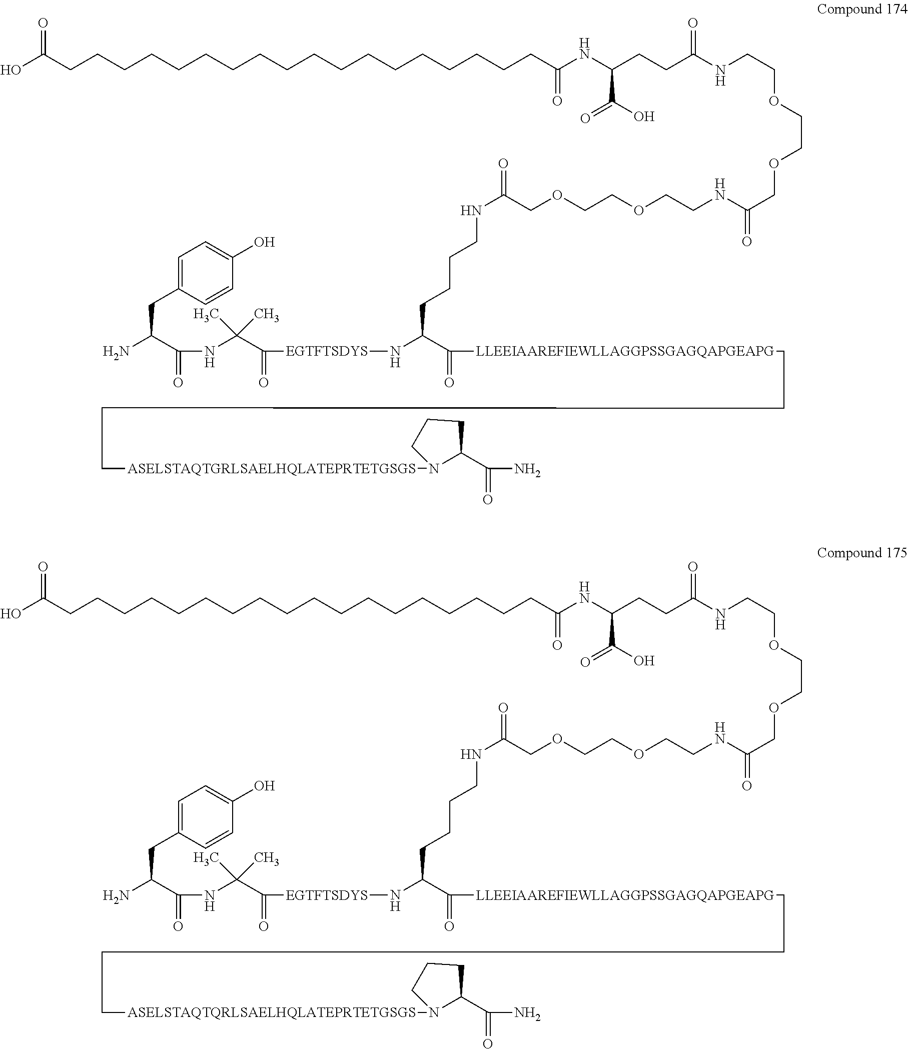

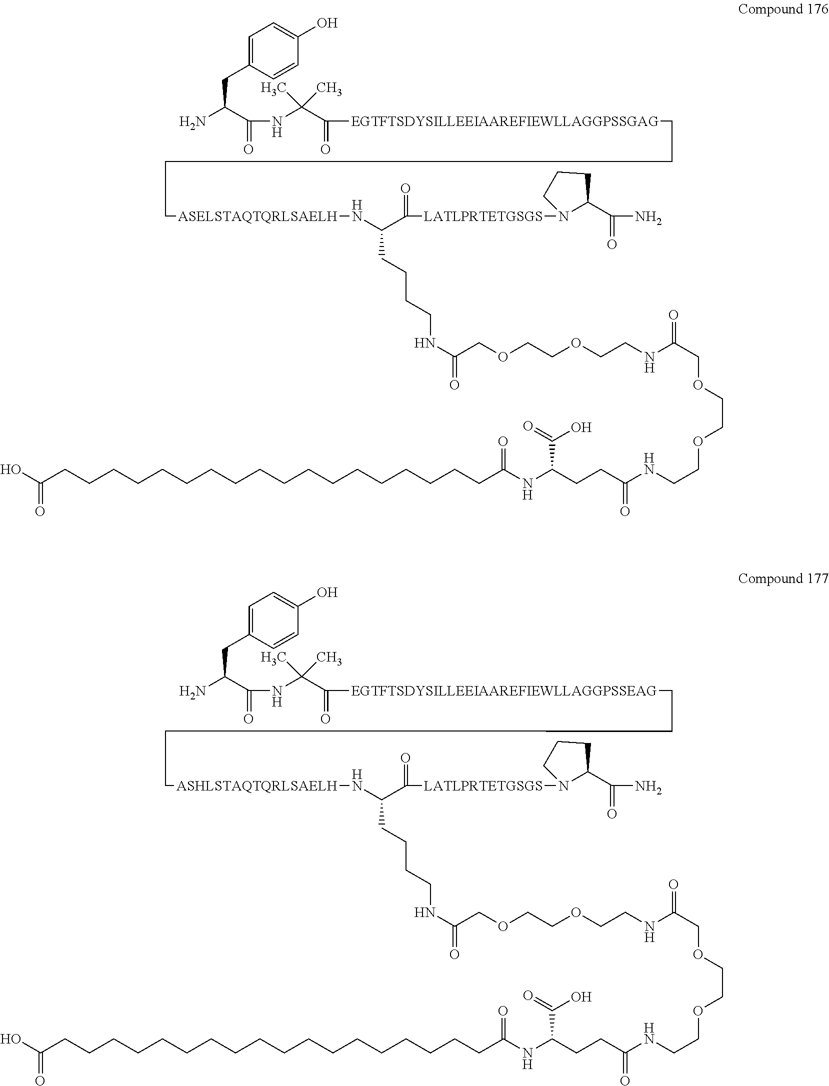

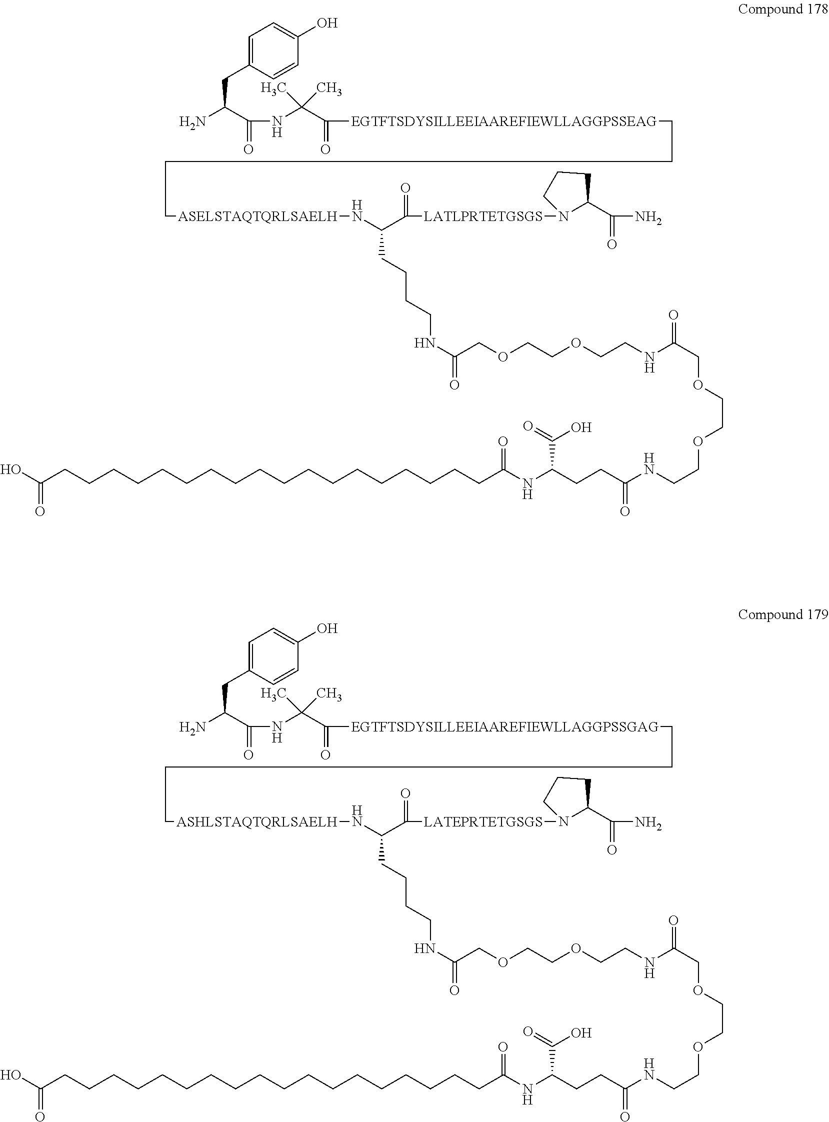

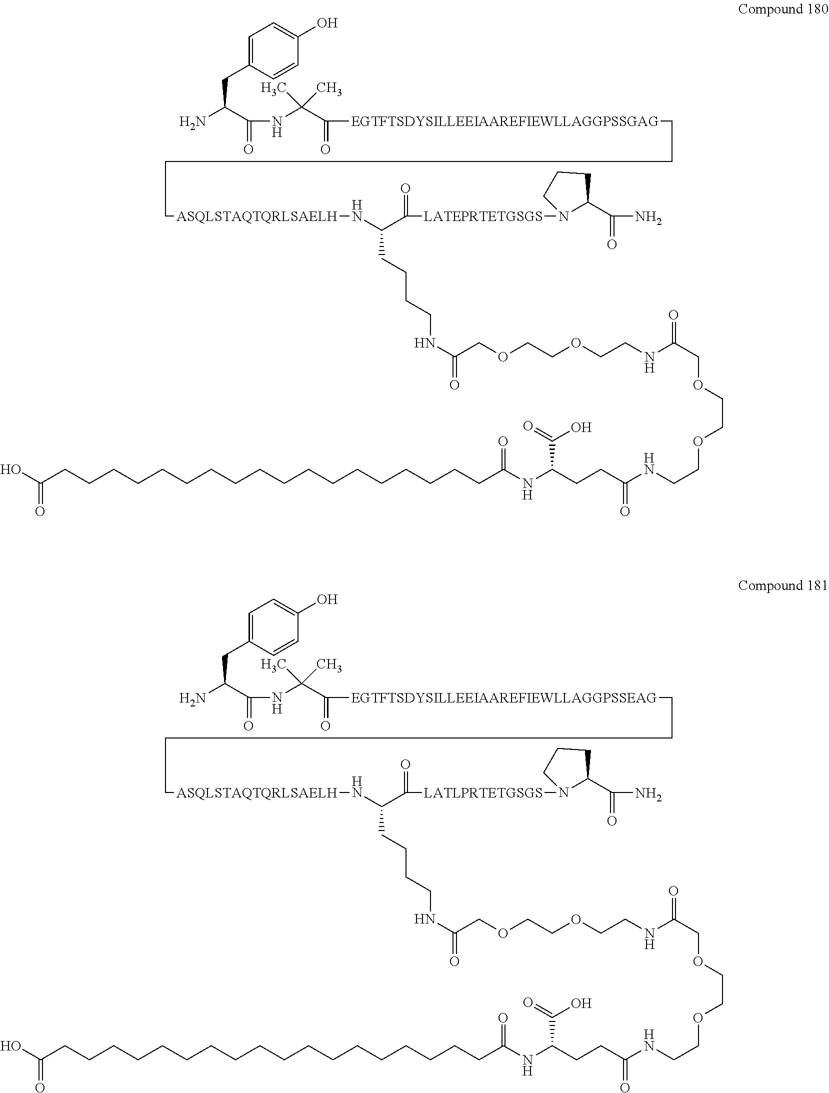

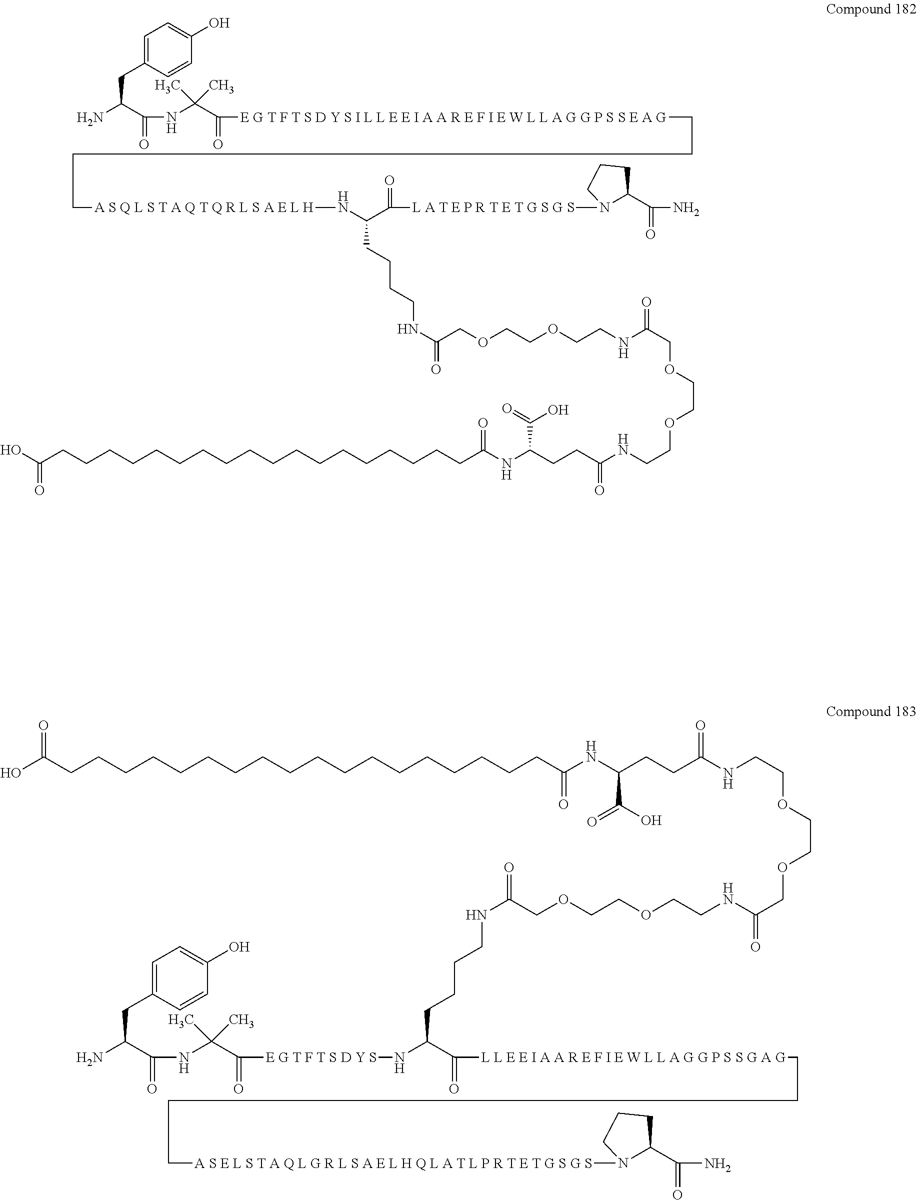

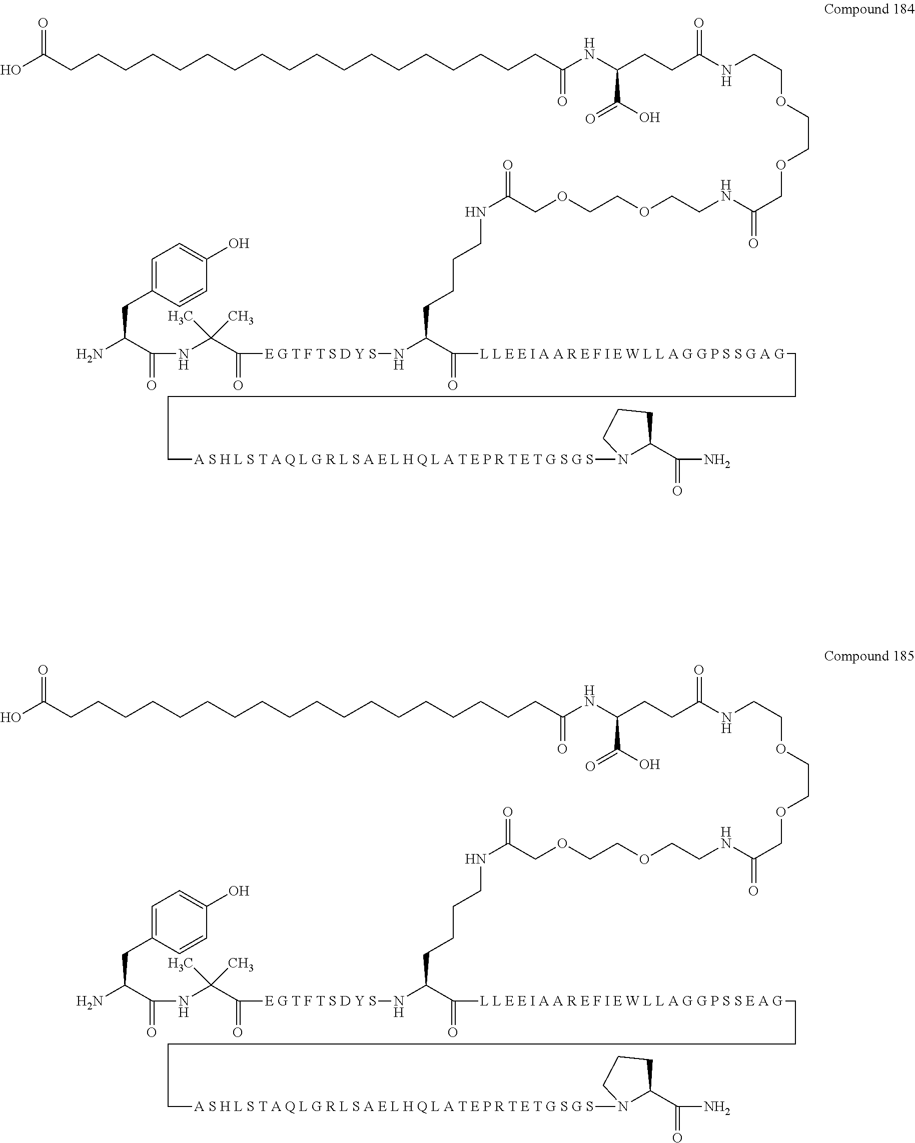

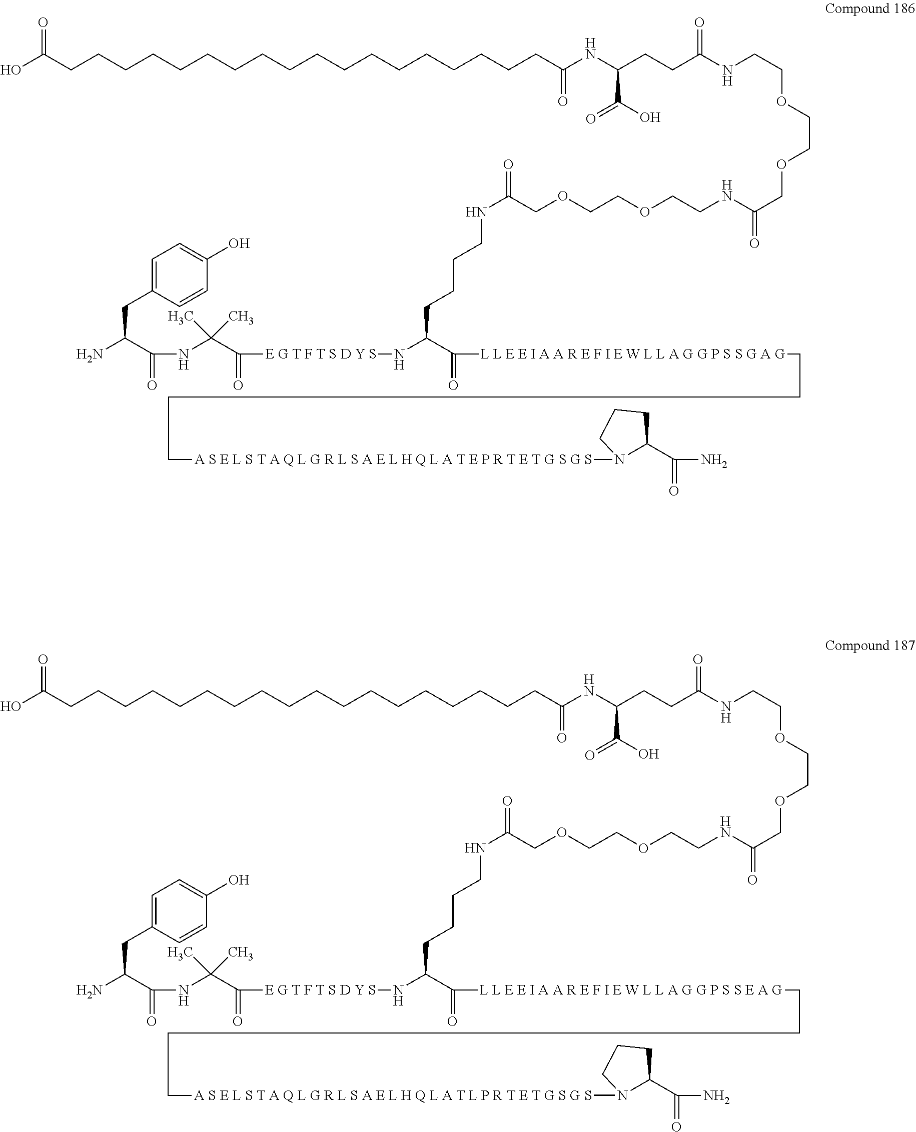

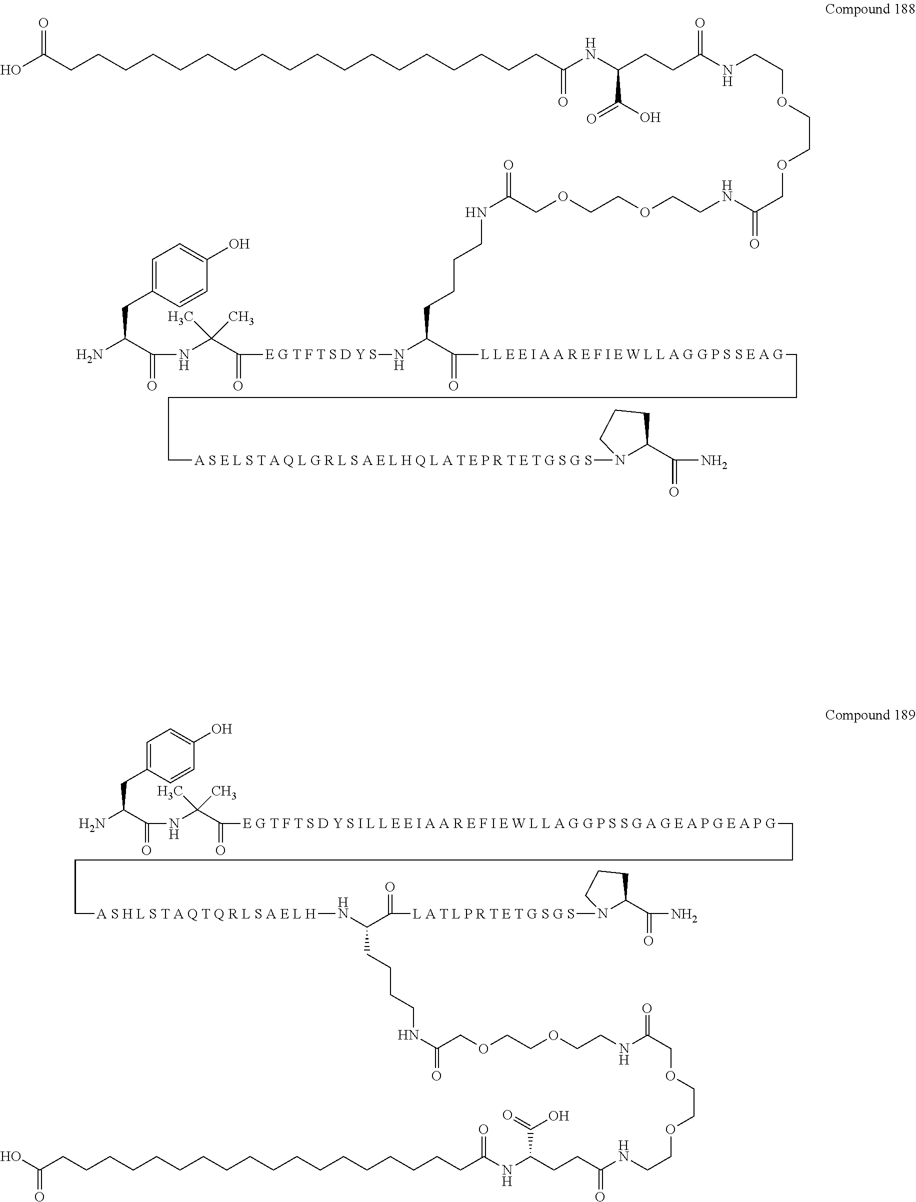

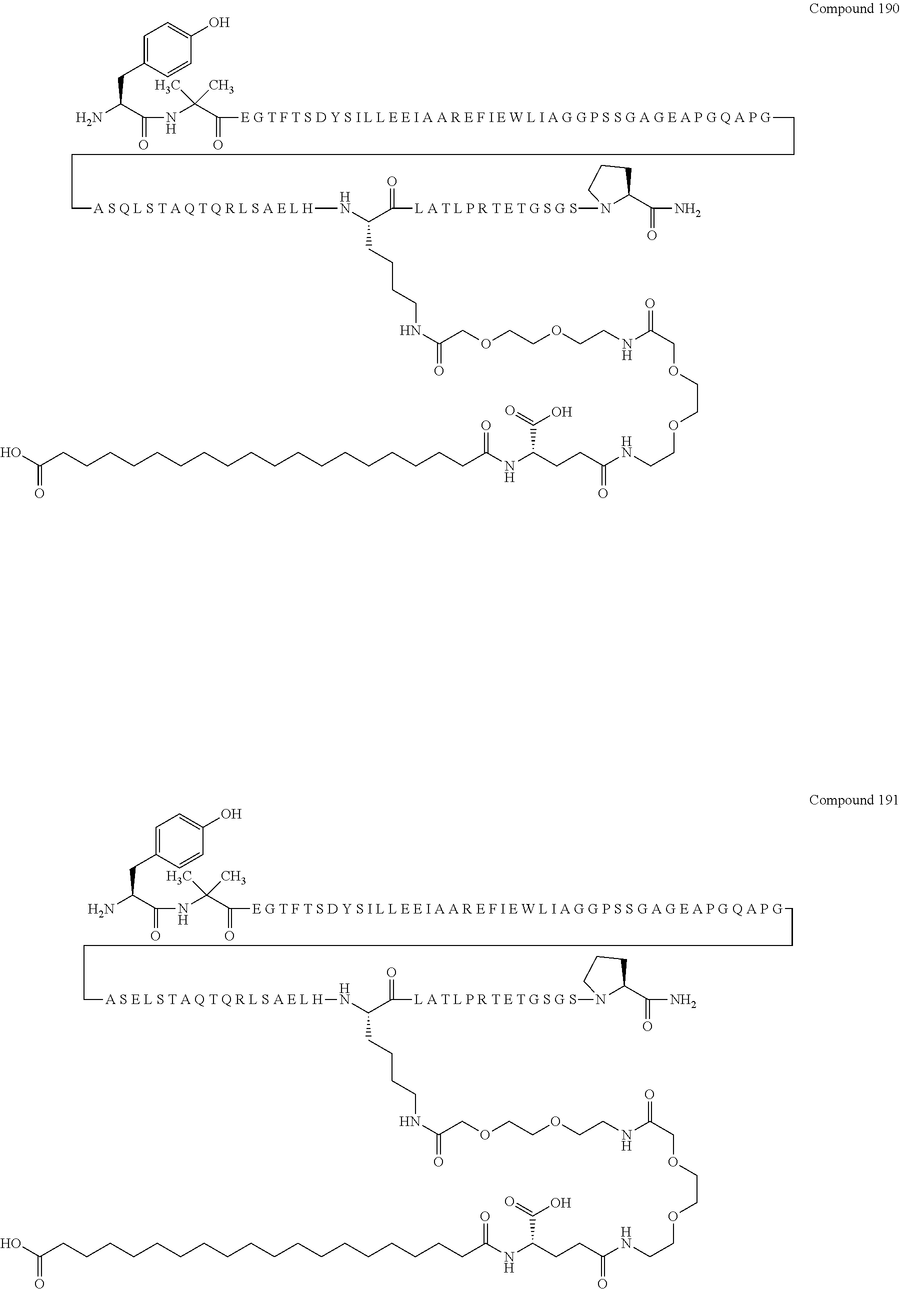

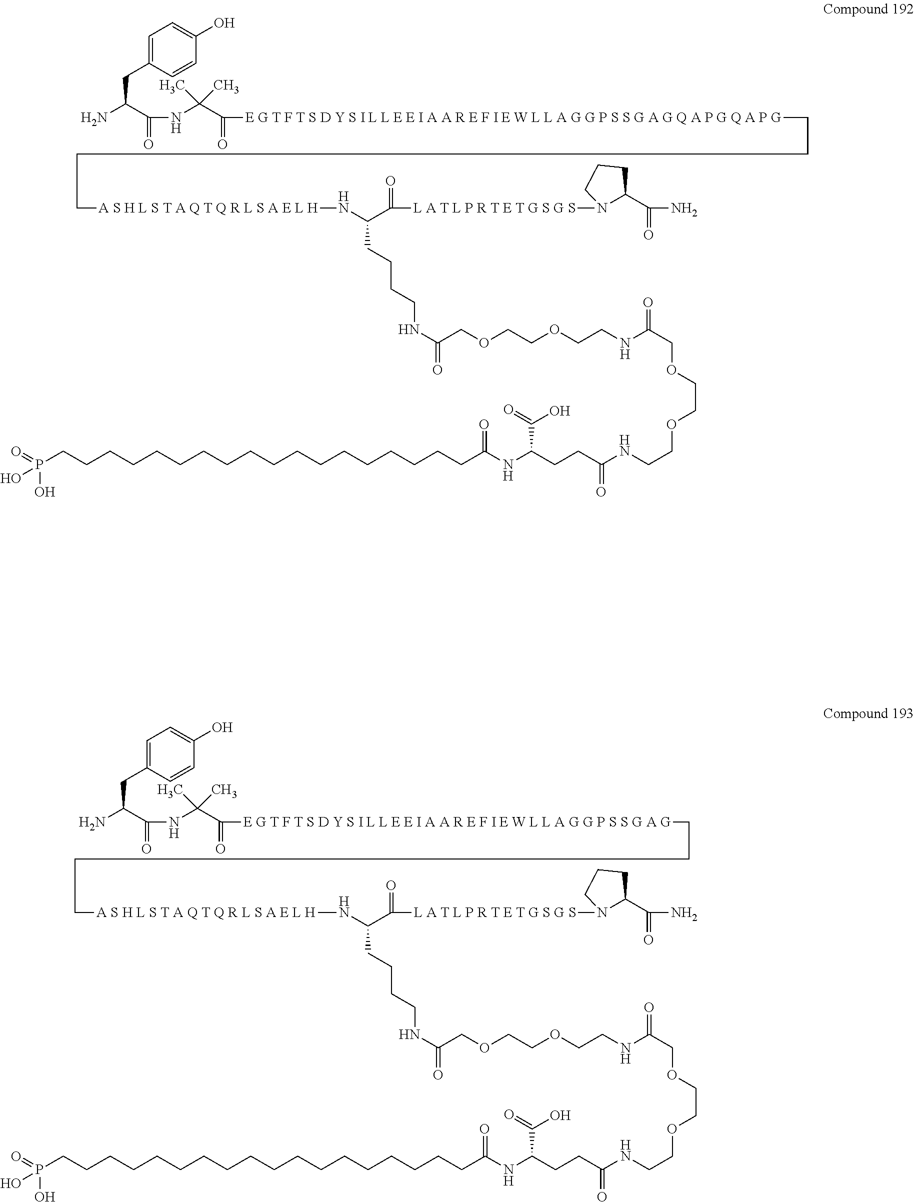

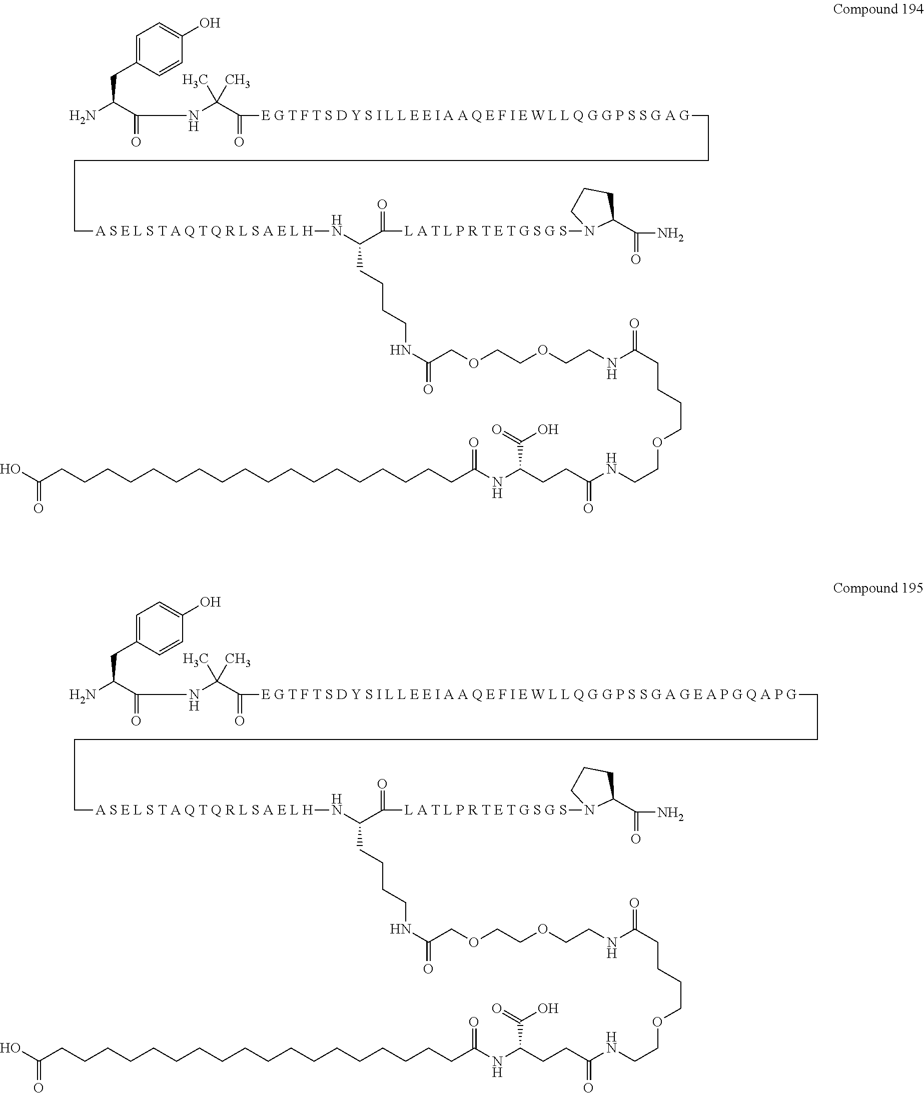

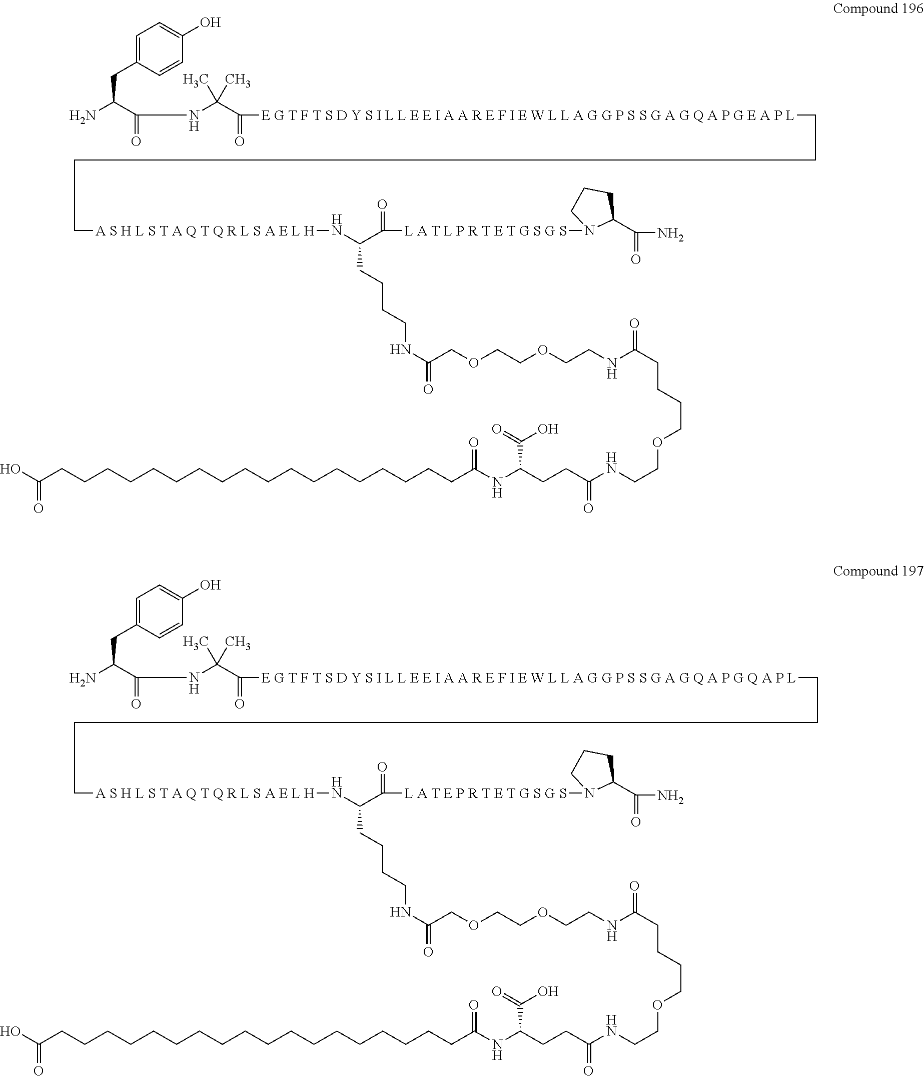

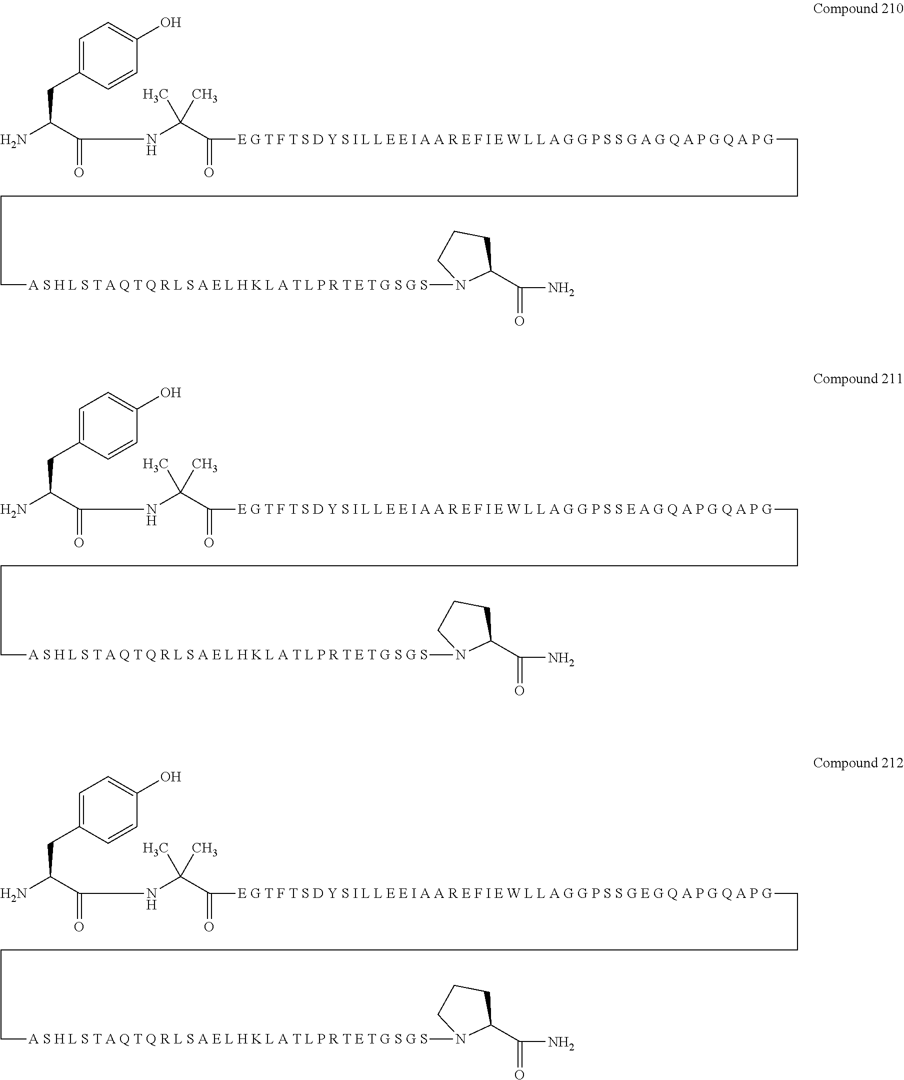

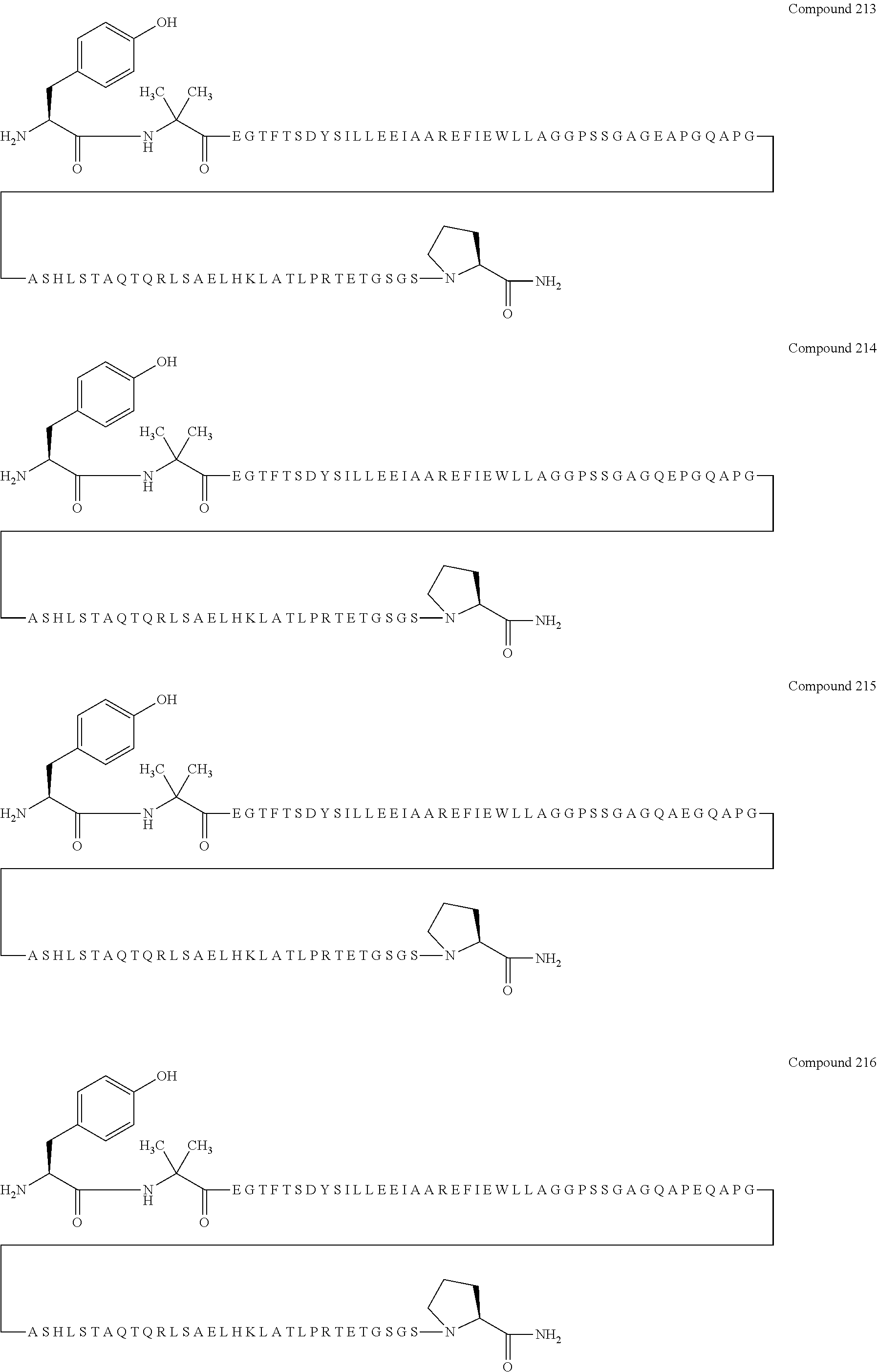

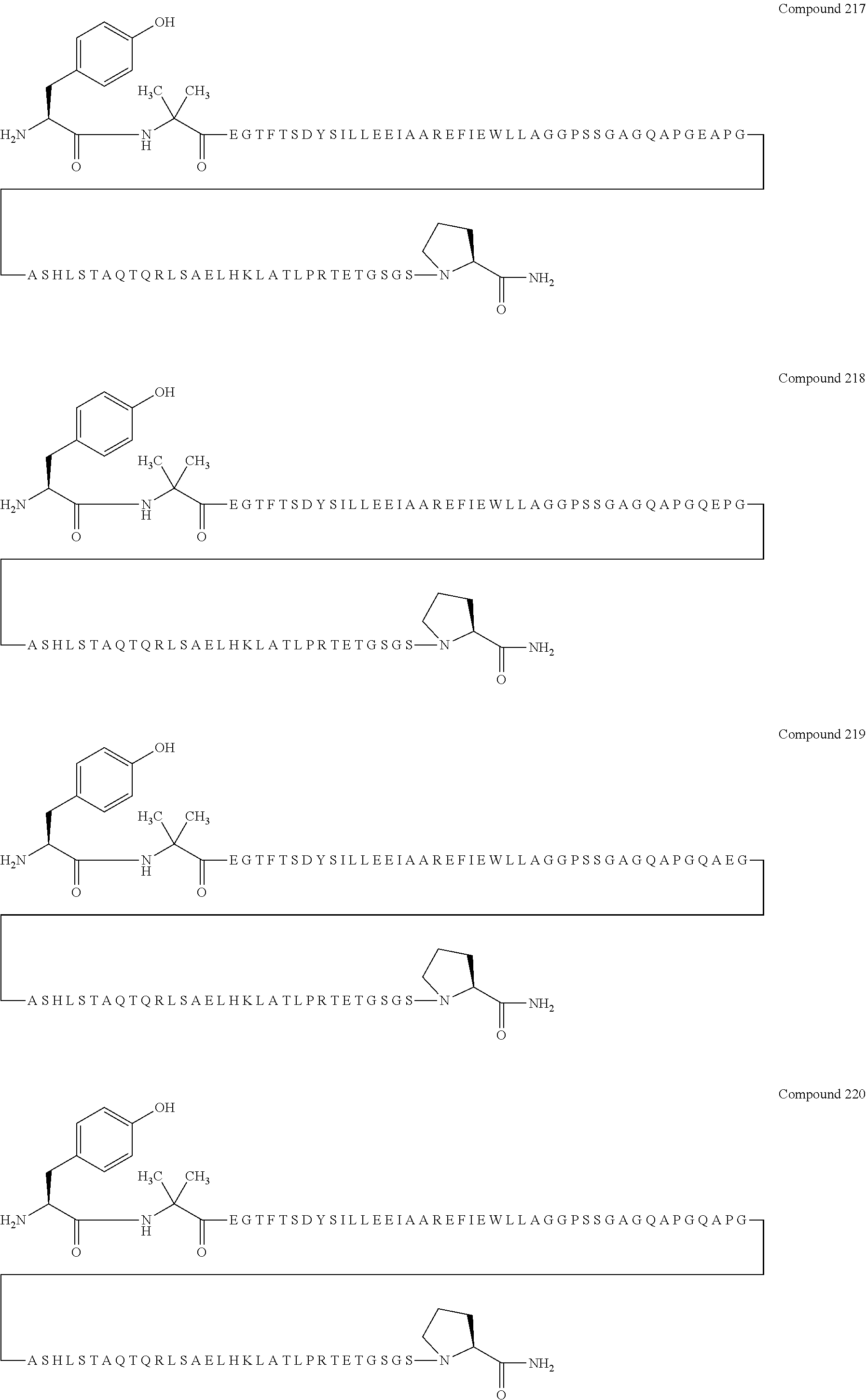

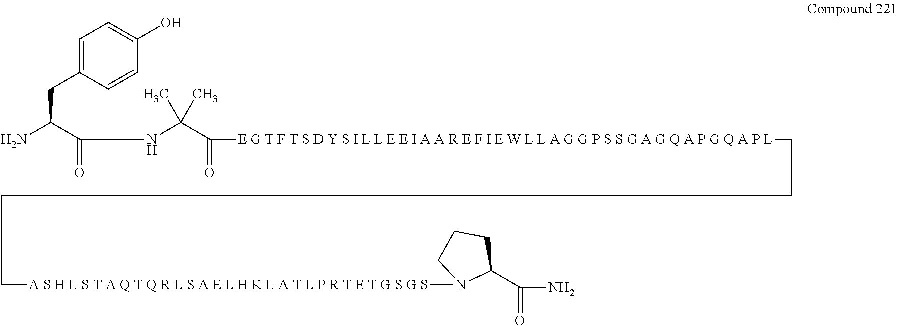

TABLE 17
Structure and amino acid sequences of the synthesized GLP-1-/GIP-/amylin-
receptor tri-agonists according to the invention.

| | SEQ | | | | |
|---|---|---|---|---|---|
| Compound | ID | | | | |
| no. | NO: | Amino acid sequences | Protraction moiety | | |
| (Table 1) | | | (Table 4) | (Table 3) | (Table 5) |
| 10 | 20 | YXEGTFTSDYSILLEEQAAREFI | Chem. 20 | Chem. 4 | Chem. 29 |
| | | EWLLAGGPSKGAPPPSGGGEA | | | |
| | | SELSTAALGRLSAELHELATLPR | | | |
| | | TETGSGSP | | | |
| 11 | 21 | YXEGTFTSDYSILLEEQAAREFI | Chem. 20 | Chem. 4 | Chem. 29 |
| | | EWLLAGGPSKGEASELSTAALG | | | |
| | | RLSAELHELATLPRTETGSGSP | | | |
| 12 | 22 | HXHGTFTSDYSILLEEQAAREFI | Chem. 20 | Chem. 4 | Chem. 29 |
| | | EWLLAGGPSKGAPPPSGGGEA | | | |
| | | SELSTAALGRLSAELHELATLPR | | | |
| | | TETGSGSP | | | |
| 13 | 23 | YXEGTFTSDYSILLEEQAAREFI | Chem. 20 | Chem. 5 | Chem. 28 |
| | | EWLLAGGPSKGAPPPSGGGGA | | | |
| | | SELSTAALGRLSAELHRLATLPR | | | |
| | | TETGSGSP | | | |
| 14 | 24 | HXHGTFTSDYSILLEEQAAREFI | Chem. 20 | Chem. 5 | Chem. 28 |
| | | EWLLAGGPSKGAPPPSGGGGA | | | |
| | | SELSTAALGRLSAELHRLATLPR | | | |
| | | TETGSGSP | | | |
| 15 | 25 | HXHGTFTSDYSILLEEQAAREFI | Chem. 20 | Chem. 5 | Chem. 28 |
| | | EWLLAGGPSKGAPPPSGGGEA | | | |
| | | SELSTAALGRLSAELHELATLPR | | | |
| | | TETGSGSP | | | |
| 16 | 26 | HXHGTFTSDYSILLEEQAAREFI | Chem. 20 | Chem. 6 | Chem. 27 |
| | | EWLLAGGPSKGAPPPSGGGEA | | | |
| | | SELSTAALGRLSAELHELATLPR | | | |
| | | TETGSGSP | | | |
| 17 | 27 | HXHGTFTSDYSILLEEQAAREFI | Chem. 20 | Chem. 4 | Chem. 29 |
| | | EWLLAGGPSKGEASELSTAALG | | | |
| | | RLSAELHRLATLPRTETGSGSP | | | |
| 18 | 28 | YXEGTFTSDYSILLEEQAAREFI | Chem. 20 | Chem. 4 | Chem. 29 |
| | | EWLLAGGPSKGEASELSTAALG | | | |
| | | RLSAELHRLATLPRTETGSGSP | | | |
| 19 | 29 | YXEGTFTSDYSILLEEQAAREFI | Chem. 20 | Chem. 5 | Chem. 28 |
| | | EWLLAGGPSKGEASELSTAALG | | | |
| | | RLSAELHRLATLPRTETGSGSP | | | |
| 20 | 30 | YXEGTFTSDYSILLEEQAAREFI | Chem. 20 | Chem. 4 | Chem. 29 |
| | | EWLLAGGPSKGASELSTAALGR | | | |
| | | LSAELHRLATLPRTETGSGSP | | | |
| 21 | 31 | YXEGTFTSDYSILLEEQAAREFI | Chem. 20 | Chem. 5 | Chem. 28 |
| | | EWLLAGGPSKGALAQTLAQTLA | | | |
| | | SELSTAALGRLSAELHELATLPR | | | |
| | | TETGSGSP | | | |
| 22 | 32 | HXHGTFTSDYSILLEEQAAREFI | Chem. 20 | Chem. 5 | Chem. 28 |
| | | EWLLAGGPSKGAPPPSLAQTLA | | | |
| | | QTLASELSTAALGRLSAELHELA | | | |
| | | TLPRTETGSGSP | | | |
| 23 | 33 | YXEGTFTSDYSILLEEQAAREFI | Chem. 20 | Chem. 5 | Chem. 28 |
| | | EWLLAGGPSKGALAQTLGTNE | | | |
| | | ASELSTAALGRLSAELHELATLP | | | |
| | | RTETGSGSP | | | |
| 24 | 34 | YXEGTFTSDYSILLEEQAAREFI | Chem. 20 | Chem. 5 | Chem. 28 |
| | | EWLLAGGPSKGALAQTLFVNQ | | | |
| | | ASELSTAALGRLSAELHELATLP | | | |
| | | RTETGSGSP | | | |
| 25 | 35 | HXHGTFTSDYSILLEEQAAREFI | Chem. 20 | Chem. 5 | Chem. 28 |
| | | EWLLAGGPSKGEASELSTAALG | | | |
| | | RLSAELHRLATLPRTETGSGSP | | | |
| 26 | 36 | YXEGTFTSDYSILLEEQAAREFI | Chem. 20 | Chem. 5 | Chem. 28 |
| | | EWLLAGGPSKGEASELSTAALG | | | |
| | | RLSAELHELATLPRTETGSGSP | | | |
| 27 | 37 | YXEGTFTSDYSILLEEQAAREFI | Chem. 20 | Chem. 5 | Chem. 28 |
| | | EWLLAGGPSKGEASELSTAQL | | | |
| | | GRLSAELHRLATLPRTETGSGS | | | |
| | | P | | | |
| 28 | 38 | YXEGTFTSDYSILLEEQAAREFI | Chem. 20 | Chem. 5 | Chem. 28 |
| | | EWLLAGGPSKGEASELSTAALA | | | |
| | | RLSAELHRLATLPRTETGSGSP | | | |
| 29 | 39 | YXEGTFTSDYSILLEEQAAREFI | Chem. 20 | Chem. 5 | Chem. 28 |
| | | EWLLAGGPSKGEASELSTAALG | | | |
| | | RLSAELHYLATLPRTETGSGSP | | | |
| 30 | 40 | HXHGTFTSDYSILLEEQAAREFI | Chem. 20 | Chem. 5 | Chem. 28 |
| | | EWLLAGGPSKGAPPPSGGGEA | | | |
| | | SELSTAALGRLSAELHRLATLPR | | | |
| | | TETGSGSP | | | |
| 31 | 41 | YXEGTFTSDYSILLEEQAAREFI | Chem. 20 | Chem. 5 | Chem. 28 |
| | | EWLLAGGPSSKAPPPSGGGEA | | | |
| | | SELSTAALGRLSAELHRLATLPR | | | |
| | | TETGSGSP | | | |
| 32 | 42 | YXEGTFTSDYSILLEEQAAREFI | Chem. 20 | Chem. 5 | Chem. 28 |
| | | EWLLAGGPSKGAEASELSTAAL | | | |
| | | GRLSAELHELATLPRTETGSGS | | | |
| | | P | | | |
| 33 | 43 | YXEGTFTSDYSILLEEQAAREFI | Chem. 20 | Chem. 5 | Chem. 28 |
| | | EWLLAGGPSKGAPPEASELSTA | | | |
| | | ALGRLSAELHELATLPRTETGS | | | |
| | | GSP | | | |
| 34 | 44 | YXEGTFTSDYSILLEEQAAREFI | Chem. 20 | Chem. 5 | Chem. 28 |
| | | EWLLAGGPSKGEASHLSTAQT | | | |
| | | QRLSAELHELATLPRTETGSGS | | | |
| | | P | | | |
| 35 | 45 | YXEGTFTSDYSILLEEQAAREFI | Chem. 20 | Chem. 5 | Chem. 28 |
| | | EWLLAGGPSKGEASHLSTAQT | | | |
| | | ARLSAELHELATLPRTETGSGS | | | |
| | | P | | | |
| 36 | 46 | HXHGTFTSDYSILLEEQAAREFI | Chem. 20 | Chem. 5 | Chem. 28 |
| | | EWLLAGGPSKGEASHLSTAQT | | | |
| | | ARLSAELHELATLPRTETGSGS | | | |
| | | P | | | |
| 37 | 47 | HXHGTFTSDYSILLEEQAAREFI | Chem. 20 | Chem. 5 | Chem. 28 |
| | | EWLLAGGPSKGAPPPSGGGEA | | | |
| | | SHLSTAQTARLSAELHRLADLP | | | |
| | | RTETGSGSP | | | |
| 38 | 48 | YXEGTFTSDYSILLEEQAAREFI | Chem. 20 | Chem. 5 | Chem. 28 |
| | | EWLLAGGPSKGGQAPGQAPGE | | | |
| | | ASHLSTAQTQRLSAELHELATL | | | |
| | | PRTETGSGSP | | | |
| 39 | 49 | YXEGTFTSDYSILLEEQAAREFI | Chem. 20 | Chem. 5 | Chem. 28 |
| | | EWLLAGGPSKGGQEPGQEPGE | | | |
| | | ASHLSTAQTQRLSAELHELATL | | | |
| | | PRTETGSGSP | | | |
| 40 | 50 | YXEGTFTSDYSILLEEQAAREFI | Chem. 20 | Chem. 5 | Chem. 28 |
| | | EWLLAGGPSKGGGGEGGGEG | | | |
| | | EASHLSTAQTQRLSAELHELAT | | | |
| | | LPRTETGSGSP | | | |
| 41 | 51 | YXEGTFTSDYSILLEEQAAREFI | Chem. 18 | Chem. 6 | Chem. 22 |
| | | EWLLAGGPSKGAPPPSGGGEA | | | |
| | | SELSTAALGRLSAELHRLADLP | | | |
| | | RTETGSGSP | | | |
| 42 | 52 | YXEGTFTSDYSILLEEQAAREFI | Chem. 18 | Chem. 6 | Chem. 22 |
| | | EWLLAGGPSSKAPPPSGGGEA | | | |
| | | SELSTAALGRLSAELHRLATLPR | | | |
| | | TETGSGSP | | | |
| 43 | 53 | HXHGTFTSDYSILLEEQAAREFI | Chem. 20 | Chem. 5 | Chem. 28 |
| | | EWLLAGGPSKGEASQLSTAAL | | | |
| | | GRLSAELHRLADLPRTETGSGS | | | |
| | | P | | | |
| 44 | 54 | HXHGTFTSDYSILLEEQAAREFI | Chem. 20 | Chem. 5 | Chem. 28 |
| | | EWLLAGGPSKGALAQTLGTNE | | | |
| | | ASELSTAALGRLSAELHRLATLP | | | |
| | | RTETGSGSP | | | |
| 45 | 55 | HXHGTFTSDYSILLEEQAAREFI | Chem. 20 | Chem. 5 | Chem. 28 |
| | | EWLLAGGPSKGALAQTLGTNE | | | |
| | | ASHLSTAQTQRLSAELHELATL | | | |
| | | PRTETGSGSP | | | |
| 46 | 56 | HXHGTFTSDYSILLEEQAAREFI | Chem. 20 | Chem. 5 | Chem. 28 |
| | | EWLLAGGPSKGAGQAPGQAPG | | | |
| | | ASHLSTAALGRLSAELHRLATLP | | | |
| | | RTETGSGSP | | | |
| 47 | 57 | HXHGTFTSDYSILLEEQAAREFI | Chem. 20 | Chem. 5 | Chem. 28 |
| | | EWLLAGGPSKGAGQAPGQAPG | | | |
| | | ASHLSTAQTQRLSAELHELATL | | | |
| | | PRTETGSGSP | | | |
| 48 | 58 | YXEGTFTSDYSILLEEQAAREFI | Chem. 20 | Chem. 5 | Chem. 28 |
| | | EWLLAGGPSSGALAQTLAQTLA | | | |
| | | SHLSTAQTQRLSAELHKLATLP | | | |
| | | RTETGSGSP | | | |
| 49 | 59 | YXEGTFTSDYSILLEEQAAREFI | Chem. 20 | Chem. 5 | Chem. 28 |
| | | EWLLAGGPSSGAPPPSGGGEA | | | |
| | | SHLSTAQTQRLSAELHKLATLP | | | |
| | | RTETGSGSP | | | |
| 50 | 60 | YXEGTFTSDYSILLEEQAAREFI | Chem. 20 | Chem. 5 | Chem. 28 |
| | | EWLLAGGPSSGEASHLSTAQT | | | |
| | | QRLSAELHKLATLPRTETGSGS | | | |
| | | P | | | |
| 51 | 61 | HXHGTFTSDYSILLEEQAAREFI | Chem. 20 | Chem. 5 | Chem. 28 |
| | | EWLLAGGPSSGALAQTLAQTLA | | | |
| | | SHLSTAQTQRLSAELHKLATLP | | | |
| | | RTETGSGSP | | | |
| 52 | 62 | YXEGTFTSDYSILLEEQAAREFI | Chem. 20 | Chem. 5 | Chem. 28 |
| | | EWLLAGGPSSGAGQAPGQAPG | | | |
| | | ASHLSTAQTQRLSAELHKLATL | | | |
| | | PRTETGSGSP | | | |
| 53 | 63 | YXEGTFTSDYSILLEEQAAREFI | Chem. 20 | Chem. 5 | Chem. 28 |
| | | EWLLAGGPSSGAGQAPGQAPG | | | |
| | | ASHLSTAALGRLSAELHKLATLP | | | |
| | | RTETGSGSP | | | |
| 54 | 64 | YXEGTFTSDYSILLEEQAAREFI | Chem. 20 | Chem. 5 | Chem. 28 |
| | | EWLLAGGPSSGAGQAPGQAPG | | | |
| | | ASKLSTAQTQRLSAELHRLATL | | | |
| | | PRTETGSGSP | | | |
| 55 | 65 | YXEGTFTSDYSILLEEQAAREFI | Chem. 20 | Chem. 5 | Chem. 28 |
| | | EWLLAGGPSSGAGQAPGQAPG | | | |
| | | ASHLSTAQTQRLSAKLHRLATL | | | |
| | | PRTETGSGSP | | | |
| 56 | 66 | YXEGTFTSDYSILLEEQAAREFI | Chem. 20 | Chem. 5 | Chem. 28 |
| | | EWLLAGGPSSGAGQAPGQAPG | | | |
| | | ASHLSTAQTQRLSAELHRLKTL | | | |
| | | PRTETGSGSP | | | |
| 57 | 67 | YXEGTFTSDYSILLEEQAAREFI | Chem. 20 | Chem. 5 | Chem. 28 |
| | | EWLLAGGPSSGAGQAPGQAPG | | | |
| | | ASHLSTAQTQRLSAELHRLATL | | | |
| | | PKTETGSGSP | | | |
| 58 | 68 | YXEGTFTSDYSILLEEQAAREFI | Chem. 20 | Chem. 5 | Chem. 28 |
| | | EWLLAGGPSSGAGQAPGQAPL | | | |
| | | ASHLSTAQTQRLSAELHKLATL | | | |
| | | PRTETGSGSP | | | |
| 59 | 69 | YXEGTFTSDYSILLEEQAAREFI | Chem. 20 | Chem. 4 | Chem. 29 |
| | | EWLLAGGPSSGALAQTLAQTLA | | | |
| | | SHLSTAQTQRLSAELHKLATLP | | | |
| | | RTETGSGSP | | | |
| 60 | 70 | YXEGTFTSDYSILLEEQAAREFI | Chem. 20 | Chem. 6 | Chem. 27 |
| | | EWLLAGGPSSGALAQTLAQTLA | | | |
| | | SHLSTAQTQRLSAELHKLATLP | | | |
| | | RTETGSGSP | | | |
| 61 | 71 | HXHGTFTSDYSILLEEQAAREFI | Chem. 20 | Chem. 4 | Chem. 29 |
| | | EWLLAGGPSKGAPPPSGGGEA | | | |
| | | SHLSTAQTARLSAELHRLADLP | | | |
| | | RTETGSGSP | | | |
| 62 | 72 | HXHGTFTSDYSILLEEQAAREFI | Chem. 20 | Chem. 4 | Chem. 29 |
| | | EWLLAGGPSKGEASQLSTAAL | | | |
| | | GRLSAELHRLADLPRTETGSGS | | | |
| | | P | | | |
| 63 | 73 | HXHGTFTSDYSILLEEQAAREFI | Chem. 20 | Chem. 5 | Chem. 28 |
| | | EWLLAGGPSKGEASQLSTAAL | | | |
| | | GRLSAELHTLATLPRTETGSGS | | | |
| | | P | | | |
| 64 | 74 | HXHGTFTSDYSILLEEQAAREFI | Chem. 20 | Chem. 5 | Chem. 28 |
| | | EWLLAGGPSKGEASQLSTAAL | | | |
| | | GRLSAELHQLATLPRTETGSGS | | | |
| | | P | | | |
| 65 | 75 | HXHGTFTSDYSILLEEQAAREFI | Chem. 20 | Chem. 5 | Chem. 28 |
| | | EWLLAGGPSKGEASQLSTAAL | | | |
| | | GRLSAELHGLATLPRTETGSGS | | | |
| | | P | | | |
| 66 | 76 | HXHGTFTSDYSILLEEQAAREFI | Chem. 20 | Chem. 5 | Chem. 28 |
| | | EWLLAGGPSKGEASQLSTAAL | | | |
| | | GRLSAELHHLATLPRTETGSGS | | | |
| | | P | | | |
| 67 | 77 | YXEGTFTSDYSILLEEQAAREFI | Chem. 20 | Chem. 5 | Chem. 28 |
| | | EWLLAGGPSRGEASELSTAALG | | | |
| | | RLSAELHKLATLPRTETGSGSP | | | |
| 68 | 78 | YXEGTFTSDYSILLEEQAAREFI | Chem. 20 | Chem. 5 | Chem. 28 |
| | | EWLLAGGPSRGEASHLSTAQT | | | |
| | | QRLSAELHKLATLPRTETGSGS | | | |
| | | P | | | |
| 69 | 79 | HXHGTFTSDYSILLEEQAAREFI | Chem. 20 | Chem. 5 | Chem. 28 |
| | | EWLLAGGPSRGEASHLSTAQT | | | |
| | | QRLSAELHKLATLPRTETGSGS | | | |
| | | P | | | |
| 70 | 80 | HXHGTFTSDYSILLEEQAAREFI | Chem. 20 | Chem. 5 | Chem. 28 |
| | | EWLLAGGPSRGAPPPSGGGEA | | | |
| | | SELSTAALGRLSAELHKLATLPR | | | |
| | | TETGSGSP | | | |
| 71 | 81 | HXHGTFTSDYSILLEEQAAREFI | Chem. 20 | Chem. 5 | Chem. 28 |
| | | EWLLAGGPSRGAPPPSGGGEA | | | |
| | | SHLSTQTQGRLSAELHKLATLP | | | |
| | | RTETGSGSP | | | |
| 72 | 82 | YXEGTFTSDYSILLEEQAAREFI | Chem. 20 | Chem. 5 | Chem. 28 |
| | | EWLLAGGPSRGAPPPSGGGEA | | | |
| | | SHLSTQTQGRLSAELHKLATLP | | | |
| | | RTETGSGSP | | | |
| 73 | 83 | HXHGTFTSDYSILLEEQAAREFI | Chem. 20 | Chem. 5 | Chem. 28 |
| | | EWLLAGGPSKGEASQLSTAAL | | | |
| | | GRLSAELHRLATLPRTETGSGS | | | |
| | | P | | | |
| 74 | 84 | YXEGTFTSDYSILLEEQAAREFI | Chem. 20 | Chem. 5 | Chem. 28 |
| | | EWLLAGGPSGGEASHLSTAQT | | | |
| | | QRLSAELHKLATLPRTETGSGS | | | |
| | | P | | | |
| 75 | 85 | HXHGTFTSDYSILLEEQAAREFI | Chem. 20 | Chem. 5 | Chem. 28 |
| | | EWLLAGGPSGGEASHLSTAQT | | | |
| | | QRLSAELHKLATLPRTETGSGS | | | |
| | | P | | | |
| 76 | 86 | YXEGTFTSDYSILLEEQAAREFI | Chem. 20 | Chem. 6 | Chem. 27 |
| | | EWLLAGGPSSGAGQAPGQAPG | | | |
| | | ASHLSTAQTQRLSAELHRLATL | | | |
| | | PKTETGSGSP | | | |
| 77 | 87 | YXEGTFTSDYSILLEEQAAREFI | Chem. 20 | Chem. 6 | Chem. 27 |
| | | EWLLAGGPSSGAGQAPGQAPL | | | |
| | | ASHLSTAQTQRLSAELHKLATL | | | |
| | | PRTETGSGSP | | | |
| 78 | 88 | HXHGTFTSDYSILLEEQAAREFI | Chem. 20 | Chem. 5 | Chem. 28 |
| | | EWLLAGGPSSGAGQAPGQAPG | | | |
| | | ASHLSTAQTQRLSAKLHRLATL | | | |
| | | PRTETGSGSP | | | |
| 79 | 89 | YXEGTFTSDYSILLEEQAAREFI | Chem. 20 | Chem. 5 | Chem. 28 |
| | | EWLLAGGPSSGEASHLSTAQT | | | |
| | | QRLSAKLHRLATLPRTETGSGS | | | |
| | | P | | | |
| 80 | 90 | YXEGTFTSDYSILLEEQAAREFI | Chem. 20 | Chem. 5 | Chem. 28 |
| | | EWLLAGGPSSGEASHLSTAQT | | | |
| | | QRLSAELHRLKTLPRTETGSGS | | | |
| | | P | | | |
| 81 | 91 | YXEGTFTSDYSILLEEQAAREFI | Chem. 20 | Chem. 5 | Chem. 28 |
| | | EWLLAGGPSSGEASHLSTAQT | | | |
| | | QRLSAELHRLATLPKTETGSGS | | | |
| | | P | | | |
| 82 | 92 | HXHGTFTSDYSILLEEQAAREFI | Chem. 20 | Chem. 5 | Chem. 28 |
| | | EWLLAGGPSSGEASHLSTAQT | | | |
| | | QRLSAKLHRLATLPRTETGSGS | | | |
| | | P | | | |
| 83 | 93 | HXHGTFTSDYSILLEEQAAREFI | Chem. 20 | Chem. 5 | Chem. 28 |
| | | EWLLAGGPSSGEASHLSTAQT | | | |
| | | QRLSAELHRLKTLPRTETGSGS | | | |
| | | P | | | |
| 84 | 94 | HXHGTFTSDYSILLEEQAAREFI | Chem. 20 | Chem. 5 | Chem. 28 |
| | | EWLLAGGPSSGEASHLSTAQT | | | |
| | | QRLSAELHRLATLPKTETGSGS | | | |
| | | P | | | |
| 85 | 95 | HXHGTFTSDYSILLEEQAAREFI | Chem. 20 | Chem. 5 | Chem. 28 |
| | | EWLLAGGPSSGEASHLSTAQT | | | |
| | | QRLSAELHKLATLPRTETGSGA | | | |
| | | P | | | |
| 86 | 96 | HXHGTFTSDYSILLEEQAAREFI | Chem. 20 | Chem. 5 | Chem. 28 |
| | | EWLLAGGPSKGAPPPSGGGAS | | | |
| | | ELSTAALGRLSAELHRLATLPRT | | | |
| | | ETGSGSP | | | |
| 87 | 97 | HXHGTFTSDYSILLEEQAAREFI | Chem. 20 | Chem. 5 | Chem. 28 |
| | | EWLLAGGPSSGEASHLSTAALG | | | |
| | | RLSAKLHRLATLPRTETGSGSP | | | |
| 88 | 98 | HXHGTFTSDYSILLEEQAAREFI | Chem. 20 | Chem. 5 | Chem. 28 |
| | | EWLLAGGPSSGEASHLSTAALG | | | |
| | | RLSAELHKLATLPRTETGSGSP | | | |
| 89 | 99 | HXHGTFTSDYSILLEEQAAREFI | Chem. 20 | Chem. 5 | Chem. 28 |
| | | EWLLAGGPSSGEASHLSTAALG | | | |
| | | RLSAELHRLKTLPRTETGSGSP | | | |
| 90 | 100 | HXHGTFTSDYSILLEEQAAREFI | Chem. 20 | Chem. 5 | Chem. 28 |
| | | EWLLAGGPSSGEASHLSTAALG | | | |
| | | RLSAELHRLATLPKTETGSGSP | | | |
| 91 | 101 | HXHGTFTSDYSILLEEQAAREFI | Chem. 20 | Chem. 5 | Chem. 28 |
| | | EWLLAGGPSSGAGQAPGQAPG | | | |
| | | ASHLSTAQTQRLSAELHRLKTL | | | |
| | | PRTETGSGSP | | | |
| 92 | 102 | HXHGTFTSDYSILLEEQAAREFI | Chem. 20 | Chem. 5 | Chem. 28 |
| | | EWLLAGGPSSGAGQAPGQAPG | | | |
| | | ASHLSTAQTQRLSAELHRLATL | | | |
| | | PKTETGSGSP | | | |
| 93 | 103 | HXHGTFTSDYSILLEEQAAREFI | Chem. 20 | Chem. 5 | Chem. 28 |
| | | EWLLAGGPSSGAGQAPGQAPL | | | |
| | | ASHLSTAQTQRLSAELHKLATL | | | |
| | | PRTETGSGSP | | | |
| 94 | 104 | YXEGTFTSDYSILLEEQAAREFI | Chem. 20 | Chem. 6 | Chem. 27 |
| | | EWLLAGGPSRGEASHLSTAQT | | | |
| | | QRLSAELHKLATLPRTETGSGS | | | |
| | | P | | | |
| 95 | 105 | YXEGTFTSDYSILLEEQAAREFI | Chem. 20 | Chem. 5 | Chem. 28 |
| | | EWLLAGGPSGGGGEASHLSTA | | | |
| | | QTQRLSAELHKLATLPRTETGS | | | |
| | | GSP | | | |
| 96 | 106 | YXEGTFTSDYSILLEEQAAREFI | Chem. 20 | Chem. 6 | Chem. 27 |
| | | EWLLAGGPSGGGGEASHLSTA | | | |
| | | QTQRLSAELHKLATLPRTETGS | | | |
| | | GSP | | | |
| 97 | 107 | YXEGTFTSDYSILLEEQAAREFI | Chem. 20 | Chem. 5 | Chem. 28 |
| | | EWLLAGGPSSGAGQAPGASHL | | | |
| | | STAQTQRLSAELHKLATLPRTE | | | |
| | | TGSGSP | | | |
| 98 | 108 | YXEGTFTSDYSILLEEQAAREFI | Chem. 20 | Chem. 5 | Chem. 28 |
| | | EWLLAGGPSSGAGQAPGQAPL | | | |
| | | ASHLSTAQTARLSAELHKLATLP | | | |
| | | RTETGSGSP | | | |
| 99 | 109 | YXEGTFTSDYSILLEEQAAREFI | Chem. 20 | Chem. 6 | Chem. 27 |
| | | EWLLAGGPSSGALQAPGQAPL | | | |
| | | ASHLSTAQTQRLSAELHKLATL | | | |
| | | PRTETGSGSP | | | |
| 100 | 110 | HXHGTFTSDYSILLEEQAAREFI | Chem. 20 | Chem. 5 | Chem. 28 |
| | | EWLLAGGPSKGAPPPSGGGEA | | | |
| | | SHLSTAQTARLSAELHRLADLP | | | |
| | | RTETGSGSP | | | |
| 101 | 111 | HXHGTFTSDYSILLEEQAAREFI | Chem. 20 | Chem. 5 | Chem. 28 |
| | | EWLLAGGPSKGAPPPSGGGEA | | | |
| | | SHLSTAQTARLSAELHQLATLP | | | |
| | | RTETGSGSP | | | |
| 102 | 112 | YXEGTFTSDYSILLEEQAAREFI | Chem. 20 | Chem. 5 | Chem. 28 |
| | | EWLLAGGPSSGAGQAPGQAPG | | | |
| | | ASHLSTAQTQRLSAELHQLKTL | | | |
| | | PRTETGSGSP | | | |
| 103 | 113 | HXHGTFTSDYSILLEEQAAREFI | Chem. 20 | Chem. 5 | Chem. 28 |
| | | EWLLAGGPSSGAGQAPGQAPG | | | |
| | | ASHLSTAQTARLSAELHKLATLP | | | |
| | | RTETGSGSP | | | |
| 104 | 114 | HXHGTFTSDYSILLEEQAAREFI | Chem. 20 | Chem. 6 | Chem. 27 |
| | | EWLLAGGPSSGAGQAPGQAPG | | | |
| | | ASHLSTAQTARLSAELHKLATLP | | | |
| | | RTETGSGSP | | | |
| 105 | 115 | YXEGTFTSDYSILLEEQAAREFI | Chem. 20 | Chem. 5 | Chem. 28 |
| | | EWLLAGGPSSGALQAPGQAPL | | | |
| | | ASHLSTAQTARLSAELHKLATLP | | | |
| | | RTETGSGSP | | | |
| 106 | 116 | HXHGTFTSDYSILLEEQAAREFI | Chem. 20 | Chem. 5 | Chem. 28 |
| | | EWLLAGGPSSGALQAPGQAPG | | | |
| | | ASHLSTAQTARLSAELHRLKTLP | | | |
| | | RTETGSGSP | | | |
| 107 | 117 | HXHGTFTSDYSILLEEQAAREFI | Chem. 20 | Chem. 5 | Chem. 28 |
| | | EWLLAGGPSSGALQAPGQAPG | | | |
| | | ASHLSTAQTARLSAELHRLATLP | | | |
| | | KTETGSGSP | | | |
| 108 | 118 | HXHGTFTSDYSILLEEQAAREFI | Chem. 20 | Chem. 5 | Chem. 28 |
| | | EWLLAGGPSSGALQAPGQAPL | | | |
| | | ASHLSTAQTARLSAELHKLATLP | | | |
| | | RTETGSGSP | | | |
| 109 | 119 | YXEGTFTSDYSILLEEQAAREFI | Chem. 20 | Chem. 5 | Chem. 28 |
| | | EWLLAGGPSSGAPPPSGGGEA | | | |
| | | SHLSTAQTQRLSAKLHRLATLP | | | |
| | | RTETGSGSP | | | |
| 110 | 120 | YXEGTFTSDYSILLEEQAAREFI | Chem. 20 | Chem. 6 | Chem. 27 |
| | | EWLLAGGPSSGAPPPSGGGEA | | | |
| | | SHLSTAQTQRLSAKLHRLATLP | | | |
| | | RTETGSGSP | | | |
| 111 | 121 | YXEGTFTSDYSILLEEQAAREFI | Chem. 21 | Chem. 5 | Chem. 31 |
| | | EWLLAGGPSSGAGQAPGQAPG | | | |
| | | ASHLSTAQTQRLSAELHRLKTL | | | |
| | | PRTETGSGSP | | | |
| 112 | 122 | HXHGTFTSDYSILLEEQAAREFI | Chem. 18 | Chem. 5 | Chem. 30 |
| | | EWLLAGGPSKGAPPPSGGGEA | | | |
| | | SHLSTAQTARLSAELHRLADLP | | | |
| | | RTETGSGSP | | | |
| 113 | 123 | YXEGTFTSDYSILLEEQAAREFI | Chem. 20 | Chem. 5 | Chem. 28 |
| | | EWLLAGGPSSGAGQAPGQAPG | | | |
| | | ASHLSTAQTARLSAELHKLATLP | | | |
| | | RTETGSGSP | | | |
| 114 | 124 | YXEGTFTSDYSILLEEQAAREFI | Chem. 20 | Chem. 5 | Chem. 28 |
| | | EWLLAGGPSSGAGQAPGQAPG | | | |
| | | ASHLSTAALARLSAELHKLATLP | | | |
| | | RTETGSGSP | | | |
| 115 | 62 | YXEGTFTSDYSILLEEQAAREFI | Chem. 20 | Chem. 6 | Chem. 27 |
| | | EWLLAGGPSSGAGQAPGQAPG | | | |
| | | ASHLSTAQTQRLSAELHKLATL | | | |
| | | PRTETGSGSP | | | |
| 120 | 170 | YXEGTFTSDYSILLEEIAAREFIE | Chem. 20 | Chem. 6 | Chem. 27 |
| | | WLLAGGPSSGAGQAPGQAPGA | | | |
| | | SHLSTAQTQRLSAELHKLATLP | | | |
| | | RTETGSGSP | | | |
| 121 | 171 | YXEGTFTSDYSILLEEIAAREFIE | Chem. 20 | Chem. 6 | Chem. 27 |
| | | WLIAGGPSSGAGQAPGQAPGA | | | |
| | | SHLSTAQTQRLSAELHKLATLP | | | |
| | | RTETGSGSP | | | |
| 122 | 172 | YXEGTFTSDYSKLLEEIAAREFI | Chem. 20 | Chem. 6 | Chem. 27 |
| | | EWLLAGGPSSGAGQAPGQAPG | | | |
| | | ASHLSTAQLGRLSAELHELATL | | | |
| | | PRTETGSGSP | | | |
| 123 | 173 | YXEGTFTSDYSKLLEEIAAREFI | Chem. 20 | Chem. 6 | Chem. 27 |
| | | EWLLAGGPSSGAGQAPGQAPG | | | |
| | | ASHLSTAQLGRLSAELHQLATL | | | |
| | | PRTETGSGSP | | | |
| 124 | 174 | YXEGTFTSDYSKLLEEIAAREFI | Chem. 20 | Chem. 6 | Chem. 27 |
| | | EWLLAGGPSSGAGQAPGQAPG | | | |
| | | ASHLSTAQTQRLSAELHELATL | | | |
| | | PRTETGSGSP | | | |
| 125 | 175 | YXEGTFTSDYSILLEEIAAREFIE | Chem. 20 | Chem. 6 | Chem. 27 |
| | | WLLAGGPSSGAGQAPGQAPLA | | | |
| | | SHLSTAQTQRLSAELHKLATLP | | | |
| | | RTETGSGSP | | | |
| 126 | 176 | YXEGTFTSDYSKLLEEIAAREFI | Chem. 20 | Chem. 6 | Chem. 27 |
| | | EWLLAGGPSSGAGQAPGQAPG | | | |
| | | ASHLSTAALGRLSAELHELATLP | | | |
| | | RTETGSGSP | | | |
| 127 | 177 | YXEGTFTSDYSKLLEEIAAREFI | Chem. 20 | Chem. 6 | Chem. 27 |
| | | EWLIAGGPSSGAGQAPGQAPG | | | |
| | | ASHLSTAALGRLSAELHELATLP | | | |
| | | RTETGSGSP | | | |
| 128 | 178 | YXEGTFTSDYSILLEEIAAREFIE | Chem. 20 | Chem. 6 | Chem. 27 |
| | | WLLAGGPSSGAGQAPGQAPLA | | | |
| | | SHLSTAQTARLSAELHKLATLPR | | | |
| | | TETGSGSP | | | |
| 129 | 179 | YXEGTFTSDYSKLLEEIAAREFI | Chem. 20 | Chem. 6 | Chem. 27 |
| | | EWLLAGGPSSGAGQAPGQAPL | | | |
| | | ASHLSTAQLGRLSAELHQLATL | | | |
| | | PRTETGSGSP | | | |
| 130 | 180 | YXEGTFTSDYSKLLEEIAAREFI | Chem. 20 | Chem. 6 | Chem. 27 |
| | | EWLLAGGPSSGAGASHLSTAQ | | | |
| | | LGRLSAELHQLATLPRTETGSG | | | |
| | | SP | | | |
| 131 | 181 | YXEGTFTSDYSILLEEIAAREFIE | Chem. 20 | Chem. 6 | Chem. 27 |
| | | WLLAGGPSSGAGASHLSTAQT | | | |
| | | QRLSAELHKLATLPRTETGSGS | | | |
| | | P | | | |
| 132 | 182 | YXEGTFTSDYSILLEEIAAREFIE | Chem. 20 | Chem. 6 | Chem. 27 |
| | | WLLAGGPSSGAGQAPGQAPLA | | | |
| | | SQLSTAQTQRLSAELHKLATLP | | | |
| | | RTETGSGSP | | | |
| 133 | 183 | YXEGTFTSDYSILLEEIAAREFIE | Chem. 20 | Chem. 6 | Chem. 27 |
| | | WLLAGGPSSGAGQAPGQAPLA | | | |
| | | SELSTAQTQRLSAELHKLATLP | | | |
| | | RTETGSGSP | | | |
| 134 | 184 | YXEGTFTSDYSILLEEIAAREFIE | Chem. 20 | Chem. 6 | Chem. 27 |
| | | WLLAGGPSSGEASHLSTAQTQ | | | |
| | | RLSAELHKLATLPRTETGSGSP | | | |
| 135 | 185 | YXEGTFTSDYSILLEEIAAREFIE | Chem. 20 | Chem. 6 | Chem. 27 |
| | | WLLAGGPSSGAGQAPGQAPAA | | | |
| | | SHLSTAQTQRLSAELHKLATLP | | | |
| | | RTETGSGSP | | | |
| 136 | 186 | YXEGTFTSDYSILLEEIAAREFIE | Chem. 20 | Chem. 6 | Chem. 27 |
| | | WLLAGGPSSGAGQAPGQAPEA | | | |
| | | SHLSTAQTQRLSAELHKLATLP | | | |
| | | RTETGSGSP | | | |
| 137 | 187 | YXEGTFTSDYSILLEEIAAREFIE | Chem. 20 | Chem. 6 | Chem. 27 |
| | | WLLAGGPSSGAGQAPGQAPPA | | | |
| | | SHLSTAQTQRLSAELHKLATLP | | | |
| | | RTETGSGSP | | | |
| 138 | 188 | YXEGTFTSDYSILLEEIAAREFIE | Chem. 20 | Chem. 6 | Chem. 27 |
| | | WLLAGGPSSGAGQAPGQAPSA | | | |
| | | SHLSTAQTQRLSAELHKLATLP | | | |
| | | RTETGSGSP | | | |
| 139 | 189 | YXEGTFTSDYSILLEEIAAREFIE | Chem. 20 | Chem. 6 | Chem. 27 |
| | | WLLAGGPSSGAGQAPGQAPVA | | | |
| | | SHLSTAQTQRLSAELHKLATLP | | | |
| | | RTETGSGSP | | | |
| 140 | 190 | YXEGTFTSDYSILLEEIAAREFIE | Chem. 20 | Chem. 6 | Chem. 27 |
| | | WLLAGGPSSGAGGGGASHLST | | | |
| | | AQTQRLSAELHKLATLPRTETG | | | |
| | | SGSP | | | |
| 141 | 191 | YXEGTFTSDYSILLEEIAAREFIE | Chem. 20 | Chem. 6 | Chem. 27 |
| | | WLLAGGPSSGAGGGGAGELST | | | |
| | | AQTQRLSAELHKLATLPRTETG | | | |
| | | SGSP | | | |
| 142 | 175 | YXEGTFTSDYSILLEEIAAREFIE | Chem. 19 | Chem. 6 | Chem. 27 |
| | | WLLAGGPSSGAGQAPGQAPLA | | | |
| | | SHLSTAQTQRLSAELHKLATLP | | | |
| | | RTETGSGSP | | | |
| 143 | 192 | YXEGTFTSDYSILLEEIAAREFIE | Chem. 20 | Chem. 6 | Chem. 27 |
| | | WLIAGGPSSGAGQAPGQAPLA | | | |
| | | SHLSTAQTQRLSAELHKLATLP | | | |
| | | RTETGSGSP | | | |
| 144 | 193 | YXEGTFTSDYSILLEEIAAREFIE | Chem. 20 | Chem. 6 | Chem. 27 |
| | | WLLAGGPSSGAGQAPGQAPGA | | | |
| | | SELSTAQTQRLSAELHKLATLP | | | |
| | | RTETGSGSP | | | |
| 145 | 194 | YXEGTFTSDYSILLEEIAAREFIE | Chem. 20 | Chem. 6 | Chem. 27 |
| | | WLLAGGPSSGAGQAPGQAPGA | | | |
| | | SELSTAQTARLSAELHKLATLPR | | | |
| | | TETGSGSP | | | |
| 146 | 195 | YXEGTFTSDYSILLEEIAAREFIE | Chem. 20 | Chem. 6 | Chem. 27 |
| | | WLLAGGPSSEAGQAPGQAPGA | | | |
| | | SHLSTAQTQRLSAELHKLATLP | | | |
| | | RTETGSGSP | | | |
| 147 | 196 | YXEGTFTSDYSILLEEIAAREFIE | Chem. 20 | Chem. 6 | Chem. 27 |
| | | WLLAGGPSSGEGQAPGQAPGA | | | |
| | | SHLSTAQTQRLSAELHKLATLP | | | |
| | | RTETGSGSP | | | |
| 148 | 197 | YXEGTFTSDYSILLEEIAAREFIE | Chem. 20 | Chem. 6 | Chem. 27 |
| | | WLLAGGPSSGAGEAPGQAPGA | | | |
| | | SHLSTAQTQRLSAELHKLATLP | | | |
| | | RTETGSGSP | | | |
| 149 | 198 | YXEGTFTSDYSILLEEIAAREFIE | Chem. 20 | Chem. 6 | Chem. 27 |
| | | WLLAGGPSSGAGQEPGQAPGA | | | |
| | | SHLSTAQTQRLSAELHKLATLP | | | |
| | | RTETGSGSP | | | |
| 150 | 199 | YXEGTFTSDYSILLEEIAAREFIE | Chem. 20 | Chem. 6 | Chem. 27 |
| | | WLLAGGPSSGAGQAEGQAPGA | | | |
| | | SHLSTAQTQRLSAELHKLATLP | | | |
| | | RTETGSGSP | | | |
| 151 | 200 | YXEGTFTSDYSILLEEIAAREFIE | Chem. 20 | Chem. 6 | Chem. 27 |
| | | WLLAGGPSSGAGQAPEQAPGA | | | |
| | | SHLSTAQTQRLSAELHKLATLP | | | |
| | | RTETGSGSP | | | |
| 152 | 201 | YXEGTFTSDYSILLEEIAAREFIE | Chem. 20 | Chem. 6 | Chem. 27 |
| | | WLLAGGPSSGAGQAPGEAPGA | | | |
| | | SHLSTAQTQRLSAELHKLATLP | | | |
| | | RTETGSGSP | | | |
| 153 | 202 | YXEGTFTSDYSILLEEIAAREFIE | Chem. 20 | Chem. 6 | Chem. 27 |
| | | WLLAGGPSSGAGQAPGQEPGA | | | |
| | | SHLSTAQTQRLSAELHKLATLP | | | |
| | | RTETGSGSP | | | |
| 154 | 203 | YXEGTFTSDYSILLEEIAAREFIE | Chem. 20 | Chem. 6 | Chem. 27 |
| | | WLLAGGPSSGAGQAPGQAEGA | | | |
| | | SHLSTAQTQRLSAELHKLATLP | | | |
| | | RTETGSGSP | | | |
| 155 | 204 | YXEGTFTSDYSILLEEIAAREFIE | Chem. 20 | Chem. 6 | Chem. 27 |
| | | WLIAGGPSSGAGQEPGQEPGA | | | |
| | | SHLSTAQTQRLSAELHKLATLP | | | |
| | | RTETGSGSP | | | |
| 156 | 205 | YXEGTFTSDYSILLEEIAAREFIA | Chem. 20 | Chem. 6 | Chem. 27 |
| | | WLIAGGPSSGAGQEPGQEPGA | | | |
| | | SHLSTAQTQRLSAELHKLATLP | | | |
| | | RTETGSGSP | | | |
| 157 | 206 | YXEGTFTSDYSILLEEIAAREFIQ | Chem. 20 | Chem. 6 | Chem. 27 |
| | | WLIAGGPSSGAGQEPGQEPGA | | | |
| | | SHLSTAQTQRLSAELHKLATLP | | | |
| | | RTETGSGSP | | | |
| 158 | 207 | YXEGTFTSDYSILLEEIAAREFIE | Chem. 20 | Chem. 6 | Chem. 27 |
| | | WLIAGGPSSGAGASHLSTAQTQ | | | |
| | | RLSAELHKLATLPRTETGSGSP | | | |
| 159 | 208 | YXEGTFTSDYSILLEEIAAREFIE | Chem. 20 | Chem. 6 | Chem. 27 |
| | | WLLAGGPSSGAGQAPGQAPAS | | | |
| | | HLSTAQTQRLSAELHKLATLPR | | | |
| | | TETGSGSP | | | |
| 160 | 170 | YXEGTFTSDYSILLEEIAAREFIE | Chem. 33 | Chem. 6 | Chem. 36 |
| | | WLLAGGPSSGAGQAPGQAPGA | | | |
| | | SHLSTAQTQRLSAELHKLATLP | | | |
| | | RTETGSGSP | | | |
| 161 | 170 | YXEGTFTSDYSILLEEIAAREFIE | Chem. 34 | Chem. 6 | Chem. 37 |
| | | WLLAGGPSSGAGQAPGQAPGA | | | |
| | | SHLSTAQTQRLSAELHKLATLP | | | |
| | | RTETGSGSP | | | |
| 162 | 209 | YXEGTFTSDYSILLEEIAAREFIE | Chem. 20 | Chem. 6 | Chem. 27 |
| | | WLLAGGPSSGAGQAPGQAPGA | | | |
| | | SHLSTAQTQRLSAELHKLATEP | | | |
| | | RTETGSGSP | | | |
| 163 | 210 | YXEGTFTSDYSILLEEIAAREFIE | Chem. 20 | Chem. 6 | Chem. 27 |
| | | WLLAGGPSSGAGQAPGQAPGA | | | |
| | | SELSTAQTQRLSAELHKLATEP | | | |
| | | RTETGSGSP | | | |
| 164 | 211 | YXEGTFTSDYSILLEEIAAREFIE | Chem. 20 | Chem. 6 | Chem. 27 |
| | | WLLAGGPSSGAGQAPGEAPGA | | | |
| | | SHLSTAQTQRLSAELHKLATEP | | | |
| | | RTETGSGSP | | | |
| 165 | 212 | YXEGTFTSDYSILLEEIAAREFIE | Chem. 20 | Chem. 6 | Chem. 27 |
| | | WLLAGGPSSEAGQAPGQAPGA | | | |
| | | SHLSTAQTQRLSAELHKLATEP | | | |
| | | RTETGSGSP | | | |
| 166 | 213 | YXEGTFTSDYSILLEEIAAREFIE | Chem. 20 | Chem. 6 | Chem. 27 |
| | | WLLAGGPSSGAGQAPGEAPGA | | | |
| | | SELSTAQTQRLSAELHKLATLP | | | |
| | | RTETGSGSP | | | |
| 167 | 214 | YXEGTFTSDYSILLEEIAAREFIE | Chem. 20 | Chem. 6 | Chem. 27 |
| | | WLLAGGPSSEAGQAPGQAPGA | | | |
| | | SELSTAQTQRLSAELHKLATLP | | | |
| | | RTETGSGSP | | | |
| 168 | 215 | YXEGTFTSDYSILLEEIAAREFIE | Chem. 20 | Chem. 6 | Chem. 27 |
| | | WLLAGGPSSEAGQAPGEAPGA | | | |
| | | SHLSTAQTQRLSAELHKLATLP | | | |
| | | RTETGSGSP | | | |
| 169 | 216 | YXEGTFTSDYSKLLEEIAAREFI | Chem. 20 | Chem. 6 | Chem. 27 |
| | | EWLLAGGPSSGAGQAPGQAPG | | | |
| | | ASELSTAALGRLSAELHELATLP | | | |
| | | RTETGSGSP | | | |
| 170 | 217 | YXEGTFTSDYSKLLEEIAAREFI | Chem. 20 | Chem. 6 | Chem. 27 |
| | | EWLLAGGPSSGAGQAPGQAPG | | | |
| | | ASELSTAALGRLSAELHQLATLP | | | |
| | | RTETGSGSP | | | |
| 171 | 218 | YXEGTFTSDYSKLLEEIAAREFI | Chem. 20 | Chem. 6 | Chem. 27 |
| | | EWLLAGGPSSGAGQAPGEAPG | | | |
| | | ASELSTAALGRLSAELHQLATLP | | | |
| | | RTETGSGSP | | | |
| 172 | 219 | YXEGTFTSDYSKLLEEIAAREFI | Chem. 20 | Chem. 6 | Chem. 27 |
| | | EWLLAGGPSSGAGQAPGEAPG | | | |
| | | ASELSTAALGRLSAELHQLATE | | | |
| | | PRTETGSGSP | | | |
| 173 | 220 | YXEGTFTSDYSKLLEEIAAREFI | Chem. 20 | Chem. 6 | Chem. 27 |
| | | EWLLAGGPSSGAGQAPGEAPG | | | |
| | | ASELSTAQLGRLSAELHQLATE | | | |
| | | PRTETGSGSP | | | |
| 174 | 221 | YXEGTFTSDYSKLLEEIAAREFI | Chem. 20 | Chem. 6 | Chem. 27 |
| | | EWLLAGGPSSGAGQAPGEAPG | | | |
| | | ASELSTAQTGRLSAELHQLATE | | | |
| | | PRTETGSGSP | | | |
| 175 | 222 | YXEGTFTSDYSKLLEEIAAREFI | Chem. 20 | Chem. 6 | Chem. 27 |
| | | EWLLAGGPSSGAGQAPGEAPG | | | |
| | | ASELSTAQTQRLSAELHQLATE | | | |
| | | PRTETGSGSP | | | |
| 176 | 223 | YXEGTFTSDYSILLEEIAAREFIE | Chem. 20 | Chem. 6 | Chem. 27 |
| | | WLLAGGPSSGAGASELSTAQT | | | |
| | | QRLSAELHKLATLPRTETGSGS | | | |
| | | P | | | |
| 177 | 224 | YXEGTFTSDYSILLEEIAAREFIE | Chem. 20 | Chem. 6 | Chem. 27 |
| | | WLLAGGPSSEAGASHLSTAQT | | | |
| | | QRLSAELHKLATLPRTETGSGS | | | |
| | | P | | | |
| 178 | 225 | YXEGTFTSDYSILLEEIAAREFIE | Chem. 20 | Chem. 6 | Chem. 27 |
| | | WLLAGGPSSEAGASELSTAQT | | | |
| | | QRLSAELHKLATLPRTETGSGS | | | |
| | | P | | | |
| 179 | 226 | YXEGTFTSDYSILLEEIAAREFIE | Chem. 20 | Chem. 6 | Chem. 27 |
| | | WLLAGGPSSGAGASHLSTAQT | | | |
| | | QRLSAELHKLATEPRTETGSGS | | | |
| | | P | | | |
| 180 | 227 | YXEGTFTSDYSILLEEIAAREFIE | Chem. 20 | Chem. 6 | Chem. 27 |
| | | WLLAGGPSSGAGASQLSTAQT | | | |
| | | QRLSAELHKLATEPRTETGSGS | | | |
| | | P | | | |
| 181 | 228 | YXEGTFTSDYSILLEEIAAREFIE | Chem. 20 | Chem. 6 | Chem. 27 |
| | | WLLAGGPSSEAGASQLSTAQT | | | |
| | | QRLSAELHKLATLPRTETGSGS | | | |
| | | P | | | |
| 182 | 229 | YXEGTFTSDYSILLEEIAAREFIE | Chem. 20 | Chem. 6 | Chem. 27 |
| | | WLLAGGPSSEAGASQLSTAQT | | | |
| | | QRLSAELHKLATEPRTETGSGS | | | |
| | | P | | | |
| 183 | 230 | YXEGTFTSDYSKLLEEIAAREFI | Chem. 20 | Chem. 6 | Chem. 27 |
| | | EWLLAGGPSSGAGASELSTAQ | | | |
| | | LGRLSAELHQLATLPRTETGSG | | | |
| | | SP | | | |
| 184 | 231 | YXEGTFTSDYSKLLEEIAAREFI | Chem. 20 | Chem. 6 | Chem. 27 |
| | | EWLLAGGPSSGAGASHLSTAQ | | | |
| | | LGRLSAELHQLATEPRTETGSG | | | |
| | | SP | | | |
| 185 | 232 | YXEGTFTSDYSKLLEEIAAREFI | Chem. 20 | Chem. 6 | Chem. 27 |
| | | EWLLAGGPSSEAGASHLSTAQL | | | |
| | | GRLSAELHQLATEPRTETGSGS | | | |
| | | P | | | |
| 186 | 233 | YXEGTFTSDYSKLLEEIAAREFI | Chem. 20 | Chem. 6 | Chem. 27 |
| | | EWLLAGGPSSGAGASELSTAQ | | | |
| | | LGRLSAELHQLATEPRTETGSG | | | |
| | | SP | | | |
| 187 | 234 | YXEGTFTSDYSKLLEEIAAREFI | Chem. 20 | Chem. 6 | Chem. 27 |
| | | EWLLAGGPSSEAGASELSTAQL | | | |
| | | GRLSAELHQLATLPRTETGSGS | | | |
| | | P | | | |
| 188 | 235 | YXEGTFTSDYSKLLEEIAAREFI | Chem. 20 | Chem. 6 | Chem. 27 |
| | | EWLLAGGPSSEAGASELSTAQL | | | |
| | | GRLSAELHQLATEPRTETGSGS | | | |
| | | P | | | |
| 189 | 236 | YXEGTFTSDYSILLEEIAAREFIE | Chem. 20 | Chem. 6 | Chem. 27 |
| | | WLLAGGPSSGAGEAPGEAPGA | | | |
| | | SHLSTAQTQRLSAELHKLATLP | | | |
| | | RTETGSGSP | | | |
| 190 | 237 | YXEGTFTSDYSILLEEIAAREFIE | Chem. 20 | Chem. 6 | Chem. 27 |
| | | WLIAGGPSSGAGEAPGQAPGA | | | |
| | | SQLSTAQTQRLSAELHKLATLP | | | |
| | | RTETGSGSP | | | |
| 191 | 238 | YXEGTFTSDYSILLEEIAAREFIE | Chem. 20 | Chem. 6 | Chem. 27 |
| | | WLIAGGPSSGAGEAPGQAPGA | | | |
| | | SELSTAQTQRLSAELHKLATLP | | | |
| | | RTETGSGSP | | | |
| 192 | 170 | YXEGTFTSDYSILLEEIAAREFIE | Chem. 20 | Chem. 32 | Chem. 35 |
| | | WLLAGGPSSGAGQAPGQAPGA | | | |
| | | SHLSTAQTQRLSAELHKLATLP | | | |
| | | RTETGSGSP | | | |
| 193 | 181 | YXEGTFTSDYSILLEEIAAREFIE | Chem. 20 | Chem. 32 | Chem. 35 |
| | | WLLAGGPSSGAGASHLSTAQT | | | |
| | | QRLSAELHKLATLPRTETGSGS | | | |
| | | P | | | |
| 194 | 239 | YXEGTFTSDYSILLEEIAAQEFIE | Chem. 20 | Chem. 6 | Chem. 27 |
| | | WLLQGGPSSGAGASELSTAQT | | | |
| | | QRLSAELHKLATLPRTETGSGS | | | |
| | | P | | | |
| 195 | 240 | YXEGTFTSDYSILLEEIAAQEFIE | Chem. 20 | Chem. 6 | Chem. 27 |
| | | WLLQGGPSSGAGEAPGQAPGA | | | |
| | | SELSTAQTQRLSAELHKLATLP | | | |
| | | RTETGSGSP | | | |
| 196 | 241 | YXEGTFTSDYSILLEEIAAREFIE | Chem. 20 | Chem. 6 | Chem. 27 |
| | | WLLAGGPSSGAGQAPGEAPLA | | | |
| | | SHLSTAQTQRLSAELHKLATLP | | | |
| | | RTETGSGSP | | | |
| 197 | 242 | YXEGTFTSDYSILLEEIAAREFIE | Chem. 20 | Chem. 6 | Chem. 27 |
| | | WLLAGGPSSGAGQAPGQAPLA | | | |
| | | SHLSTAQTQRLSAELHKLATEP | | | |
| | | RTETGSGSP | | | |

wherein X represents always Aib.

### Example 3: LCMS Characterization of the Synthesized Compounds

### LCMS Characterization Method

LCMS analysis was performed on a set up consisting of Waters Acquity UPLC H Class system and Waters Xevo G2-XS QTof. Eluents: A: Milli-Q water; B: MeCN; C: 2% formic acid+0.1% TFA in Milli-Q water.

The analysis was performed at RT (column temperature 60° C.) by injecting an appropriate volume of the sample onto the column. The sample was eluted with a linear gradient of 5-95% B in A, and constant 5% C.

The UPLC conditions, detector settings, and mass spectrometer settings were: Column: Waters Acquity BEH Shield, C-18, 1.7 μm, 2.1 mm×50 mm. Gradient: Linear 5%-95% B, and constant 5% C during 4.0 min at 0.4 ml/min. Total run-time: 7.0 min. Detection: MS sensitivity mode, ionisation method: ES. Scan: 50-5000 amu.

The monoisotopic mass was recorded for the synthesized compounds and their found and calculated values are depicted in Table 6.

TABLE 6
Measured and calculated MS species
for all synthesized compounds

| | Species | m/z found | m/z calc |
|---|---|---|---|
| Reference | [M + 5H]5+ | 1385.469 | 1385.485 |
| compound 1 | [M + 6H]6+ | 1154.892 | 1154.739 |
| Reference | [M + 5H]5+ | 1796.108 | 1795.913 |
| compound 2 | [M + 6H]6+ | 1496.760 | 1496.762 |
| Reference | [M + 5H]5+ | 1675.279 | 1675.266 |
| compound 3 | [M + 6H]6+ | 1396.379 | 1396.223 |
| Reference | [M + 3H]3+ | 1517.136 | 1517.156 |
| compound 4 | [M + 4H]4+ | 1138.116 | 1138.119 |
| Reference | [M + 3H]3+ | 1604.490 | 1604.516 |
| compound 5 | [M + 4H]4+ | 1203.626 | 1203.639 |
| Reference | [M + 3H]3+ | 1469.738 | 1469.758 |
| compound 6 | [M + 4H]4+ | 1102.568 | 1102.570 |
| Reference | [M + 3H]3+ | 1371.374 | 1371.379 |
| compound 7 | [M + 4H]4+ | 1028.789 | 1028.786 |
| Compound 10 | [M + 5H]5+ | 1681.268 | 1681.254 |
| | [M + 6H]6+ | 1401.225 | 1401.213 |
| Compound 11 | [M + 5H]5+ | 1557.203 | 1557.196 |
| | [M + 6H]6+ | 1297.849 | 1297.831 |
| Compound 12 | [M + 5H]5+ | 1677.680 | 1677.656 |
| | [M + 6H]6+ | 1398.225 | 1398.215 |
| Compound 13 | [M + 5H]5+ | 1677.881 | 1677.868 |
| | [M + 6H]6+ | 1398.408 | 1398.391 |
| Compound 14 | [M + 5H]5+ | 1674.498 | 1674.270 |
| | [M + 6H]6+ | 1395.427 | 1395.393 |
| Compound 15 | [M + 5H]5+ | 1683.281 | 1683.263 |
| | [M + 6H]6+ | 1402.894 | 1402.887 |
| Compound 16 | [M + 5H]5+ | 1688.892 | 1688.869 |
| | [M + 6H]6+ | 1407.740 | 1407.559 |
| Compound 17 | [M + 5H]5+ | 1559.027 | 1559.010 |
| | [M + 6H]6+ | 1299.352 | 1299.343 |
| Compound 18 | [M + 5H]5+ | 1562.615 | 1562.607 |
| | [M + 6H]6+ | 1302.348 | 1302.341 |
| Compound 19 | [M + 5H]5+ | 1568.232 | 1568.214 |
| | [M + 6H]6+ | 1307.017 | 1307.013 |
| Compound 20 | [M + 5H]5+ | 1536.816 | 1536.799 |
| | [M + 6H]6+ | 1280.839 | 1280.834 |
| Compound 21 | [M + 5H]5+ | 1739.141 | 1739.109 |
| | [M + 6H]6+ | 1449.597 | 1449.425 |
| Compound 22 | [M + 5H]5+ | 1811.376 | 1811.149 |
| | [M + 6H]6+ | 1509.467 | 1509.459 |
| Compound 23 | [M + 5H]5+ | 1736.720 | 1736.694 |
| | [M + 6H]6+ | 1447.432 | 1447.413 |
| Compound 24 | [M + 5H]5+ | 1754.324 | 1754.111 |
| | [M + 6H]6+ | 1461.943 | 1461.927 |
| Compound 25 | [M + 5H]5+ | 1564.734 | 1564.616 |
| | [M + 6H]6+ | 1304.031 | 1304.015 |
| Compound 26 | [M + 5H]5+ | 1562.809 | 1562.802 |
| | [M + 6H]6+ | 1302.510 | 1302.503 |
| Compound 27 | [M + 5H]5+ | 1579.625 | 1579.618 |
| | [M + 6H]6+ | 1316.529 | 1316.516 |
| Compound 28 | [M + 5H]5+ | 1571.036 | 1571.017 |
| | [M + 6H]6+ | 1309.355 | 1309.349 |
| Compound 29 | [M + 5H]5+ | 1569.625 | 1569.606 |
| | [M + 6H]6+ | 1308.186 | 1308.173 |
| Compound 30 | [M + 5H]5+ | 1688.690 | 1688.674 |
| | [M + 6H]6+ | 1407.403 | 1407.397 |
| Compound 31 | [M + 5H]5+ | 1698.488 | 1698.274 |
| | [M + 6H]6+ | 1415.410 | 1415.396 |
| Compound 32 | [M + 5H]5+ | 1577.025 | 1577.009 |
| | [M + 6H]6+ | 1314.348 | 1314.342 |
| Compound 33 | [M + 5H]5+ | 1615.844 | 1615.830 |
| | [M + 6H]6+ | 1346.699 | 1346.693 |
| Compound 34 | [M + 5H]5+ | 1587.635 | 1587.610 |
| | [M + 6H]6+ | 1323.187 | 1323.176 |
| Compound 35 | [M + 5H]5+ | 1576.229 | 1576.205 |
| | [M + 6H]6+ | 1313.681 | 1313.672 |
| Compound 36 | [M + 5H]5+ | 1572.625 | 1572.608 |
| | [M + 6H]6+ | 1310.687 | 1310.674 |
| Compound 37 | [M + 5H]5+ | 1704.884 | 1704.874 |
| | [M + 6H]6+ | 1420.910 | 1420.896 |
| Compound 38 | [M + 5H]5+ | 1740.302 | 1740.282 |
| | [M + 6H]6+ | 1450.578 | 1450.403 |
| Compound 39 | [M + 5H]5+ | 1763.503 | 1763.484 |
| | [M + 6H]6+ | 1469.759 | 1469.738 |
| Compound 40 | [M + 5H]5+ | 1719.068 | 1719.057 |
| | [M + 6H]6+ | 1432.731 | 1432.715 |
| Compound 41 | [M + 5H]5+ | 1642.859 | 1642.645 |
| | [M + 6H]6+ | 1369.054 | 1369.038 |
| Compound 42 | [M + 5H]5+ | 1645.862 | 1645.851 |
| | [M + 6H]6+ | 1371.719 | 1371.710 |
| Compound 43 | [M + 5H]5+ | 1567.228 | 1567.215 |
| | [M + 6H]6+ | 1306.189 | 1306.180 |
| Compound 44 | [M + 5H]5+ | 1738.732 | 1738.508 |
| | [M + 6H]6+ | 1449.098 | 1448.925 |
| Compound 45 | [M + 5H]5+ | 1757.924 | 1757.904 |
| | [M + 6H]6+ | 1465.104 | 1465.088 |
| Compound 46 | [M + 5H]5+ | 1707.503 | 1707.290 |
| | [M + 6H]6+ | 1423.085 | 1422.910 |
| Compound 47 | [M + 5H]5+ | 1725.299 | 1725.083 |
| | [M + 6H]6+ | 1437.908 | 1437.737 |
| Compound 48 | [M + 5H]5+ | 1755.541 | 1755.514 |
| | [M + 6H]6+ | 1463.116 | 1463.096 |
| Compound 49 | [M + 5H]5+ | 1703.280 | 1703.266 |
| | [M + 6H]6+ | 1419.569 | 1419.556 |
| Compound 50 | [M + 5H]5+ | 1579.219 | 1579.208 |
| | [M + 6H]6+ | 1316.188 | 1316.174 |
| Compound 51 | [M + 5H]5+ | 1752.131 | 1751.917 |
| | [M + 6H]6+ | 1460.270 | 1460.098 |
| Compound 52 | [M + 5H]5+ | 1720.290 | 1720.279 |
| | [M + 6H]6+ | 1433.738 | 1433.733 |
| Compound 53 | [M + 5H]5+ | 1697.089 | 1697.074 |
| | [M + 6H]6+ | 1414.410 | 1414.396 |
| Compound 54 | [M + 5H]5+ | 1724.110 | 1724.087 |
| | [M + 6H]6+ | 1436.915 | 1436.907 |
| Compound 55 | [M + 5H]5+ | 1725.707 | 1725.690 |
| | [M + 6H]6+ | 1438.420 | 1438.243 |
| Compound 56 | [M + 5H]5+ | 1737.316 | 1737.291 |
| | [M + 6H]6+ | 1447.931 | 1447.911 |
| Compound 57 | [M + 5H]5+ | 1720.494 | 1720.279 |
| | [M + 6H]6+ | 1433.908 | 1433.733 |
| Compound 58 | [M + 5H]5+ | 1731.507 | 1731.491 |
| | [M + 6H]6+ | 1443.094 | 1443.077 |
| Compound 59 | [M + 5H]5+ | 1749.923 | 1749.908 |
| | [M + 6H]6+ | 1458.442 | 1458.425 |
| Compound 60 | [M + 5H]5+ | 1761.133 | 1761.120 |
| | [M + 6H]6+ | 1467.783 | 1467.768 |
| Compound 61 | [M + 5H]5+ | 1699.483 | 1699.267 |
| | [M + 6H]6+ | 1416.241 | 1416.224 |
| Compound 62 | [M + 5H]5+ | 1561.823 | 1561.609 |
| | [M + 6H]6+ | 1301.521 | 1301.509 |
| Compound 63 | [M + 5H]5+ | 1553.427 | 1553.409 |
| | [M + 6H]6+ | 1295.021 | 1294.675 |
| Compound 64 | [M + 5H]5+ | 1559.027 | 1558.811 |
| | [M + 6H]6+ | 1299.190 | 1299.177 |
| Compound 65 | [M + 5H]5+ | 1544.620 | 1544.603 |
| | [M + 6H]6+ | 1287.347 | 1287.337 |
| Compound 66 | [M + 5H]5+ | 1560.627 | 1560.611 |
| | [M + 6H]6+ | 1300.680 | 1300.677 |
| Compound 67 | [M + 5H]5+ | 1568.426 | 1568.214 |
| | [M + 6H]6+ | 1307.017 | 1307.013 |
| Compound 68 | [M + 5H]5+ | 1593.035 | 1593.021 |
| | [M + 6H]6+ | 1327.700 | 1327.686 |
| Compound 69 | [M + 5H]5+ | 1589.444 | 1589.424 |
| | [M + 6H]6+ | 1324.870 | 1324.688 |
| Compound 70 | [M + 5H]5+ | 1688.690 | 1688.674 |
| | [M + 6H]6+ | 1407.572 | 1407.397 |
| Compound 71 | [M + 5H]5+ | 1710.700 | 1710.679 |
| | [M + 6H]6+ | 1425.741 | 1425.734 |
| Compound 72 | [M + 5H]5+ | 1714.492 | 1714.277 |
| | [M + 6H]6+ | 1428.739 | 1428.732 |
| Compound 73 | [M + 5H]5+ | 1564.427 | 1564.419 |
| | [M + 6H]6+ | 1303.854 | 1303.851 |
| Compound 74 | [M + 5H]5+ | 1573.420 | 1573.205 |
| | [M + 6H]6+ | 1311.176 | 1311.172 |
| Compound 75 | [M + 5H]5+ | 1569.625 | 1569.608 |
| | [M + 6H]6+ | 1308.186 | 1308.174 |
| Compound 76 | [M + 5H]5+ | 1725.911 | 1725.885 |
| | [M + 6H]6+ | 1438.420 | 1438.405 |
| Compound 77 | [M + 5H]5+ | 1737.112 | 1737.097 |
| | [M + 6H]6+ | 1447.759 | 1447.749 |
| Compound 78 | [M + 5H]5+ | 1722.310 | 1722.093 |
| | [M + 6H]6+ | 1435.256 | 1435.245 |
| Compound 79 | [M + 5H]5+ | 1584.637 | 1584.619 |
| | [M + 6H]6+ | 1320.689 | 1320.684 |
| Compound 80 | [M + 5H]5+ | 1596.236 | 1596.220 |
| | [M + 6H]6+ | 1330.355 | 1330.351 |
| Compound 81 | [M + 5H]5+ | 1579.219 | 1579.208 |
| | [M + 6H]6+ | 1316.188 | 1316.174 |
| Compound 82 | [M + 5H]5+ | 1581.235 | 1581.022 |
| | [M + 6H]6+ | 1317.851 | 1317.686 |
| Compound 83 | [M + 5H]5+ | 1592.839 | 1592.623 |
| | [M + 6H]6+ | 1327.357 | 1327.353 |
| Compound 84 | [M + 5H]5+ | 1575.628 | 1575.610 |
| | [M + 6H]6+ | 1313.192 | 1313.176 |
| Compound 85 | [M + 5H]5+ | 1572.431 | 1572.411 |
| | [M + 6H]6+ | 1310.524 | 1310.510 |
| Compound 86 | [M + 5H]5+ | 1662.884 | 1662.866 |
| | [M + 6H]6+ | 1386.060 | 1385.889 |
| Compound 87 | [M + 5H]5+ | 1557.832 | 1557.817 |
| | [M + 6H]6+ | 1298.527 | 1298.349 |
| Compound 88 | [M + 5H]5+ | 1552.412 | 1552.405 |
| | [M + 6H]6+ | 1293.843 | 1293.839 |
| Compound 89 | [M + 5H]5+ | 1569.431 | 1569.418 |
| | [M + 6H]6+ | 1308.023 | 1308.016 |
| Compound 90 | [M + 5H]5+ | 1552.412 | 1552.405 |
| | [M + 6H]6+ | 1293.843 | 1293.839 |
| Compound 91 | [M + 5H]5+ | 1733.908 | 1733.694 |
| | [M + 6H]6+ | 1444.928 | 1444.913 |
| Compound 92 | [M + 5H]5+ | 1716.898 | 1716.681 |
| | [M + 6H]6+ | 1430.750 | 1430.735 |
| Compound 93 | [M + 5H]5+ | 1727.917 | 1727.894 |
| | [M + 6H]6+ | 1440.096 | 1440.079 |
| Compound 94 | [M + 5H]5+ | 1598.656 | 1598.628 |
| | [M + 6H]6+ | 1332.534 | 1332.358 |
| Compound 95 | [M + 5H]5+ | 1596.024 | 1596.014 |
| | [M + 6H]6+ | 1330.191 | 1330.180 |
| Compound 96 | [M + 5H]5+ | 1601.635 | 1601.620 |
| | [M + 6H]6+ | 1334.865 | 1334.851 |
| Compound 97 | [M + 5H]5+ | 1649.665 | 1649.645 |
| | [M + 6H]6+ | 1375.040 | 1374.872 |
| Compound 98 | [M + 5H]5+ | 1720.120 | 1720.087 |
| | [M + 6H]6+ | 1433.598 | 1433.574 |
| Compound 99 | [M + 5H]5+ | 1742.914 | 1742.704 |
| | [M + 6H]6+ | 1452.433 | 1452.421 |
| Compound 100 | [M + 5H]5+ | 1710.497 | 1710.480 |
| | [M + 6H]6+ | 1425.741 | 1425.568 |
| Compound 101 | [M + 5H]5+ | 1696.685 | 1696.469 |
| | [M + 6H]6+ | 1414.071 | 1413.892 |
| Compound 102 | [M + 5H]5+ | 1731.898 | 1731.683 |
| | [M + 6H]6+ | 1443.249 | 1443.237 |
| Compound 103 | [M + 5H]5+ | 1705.492 | 1705.277 |
| | [M + 6H]6+ | 1421.403 | 1421.232 |
| Compound 104 | [M + 5H]5+ | 1711.106 | 1710.883 |
| | [M + 6H]6+ | 1425.926 | 1425.904 |
| Compound 105 | [M + 5H]5+ | 1731.320 | 1731.299 |
| | [M + 6H]6+ | 1442.923 | 1442.917 |
| Compound 106 | [M + 5H]5+ | 1733.533 | 1733.502 |
| | [M + 6H]6+ | 1444.772 | 1444.753 |
| Compound 107 | [M + 5H]5+ | 1716.508 | 1716.489 |
| | [M + 6H]6+ | 1430.595 | 1430.576 |
| Compound 108 | [M + 5H]5+ | 1727.917 | 1727.702 |
| | [M + 6H]6+ | 1440.096 | 1439.919 |
| Compound 109 | [M + 5H]5+ | 1708.890 | 1708.678 |
| | [M + 6H]6+ | 1424.243 | 1424.066 |
| Compound 110 | [M + 5H]5+ | 1714.289 | 1714.284 |
| | [M + 6H]6+ | 1428.909 | 1428.738 |
| Compound 111 | [M + 5H]5+ | 1766.339 | 1766.306 |
| | [M + 6H]6+ | 1472.112 | 1472.090 |
| Compound 112 | [M + 5H]5+ | 1646.858 | 1646.844 |
| | [M + 6H]6+ | 1372.553 | 1372.538 |
| Compound 113 | [M + 5H]5+ | 1708.890 | 1708.874 |
| | [M + 6H]6+ | 1424.243 | 1424.230 |
| Compound 114 | [M + 5H]5+ | 1700.090 | 1699.877 |
| | [M + 6H]6+ | 1416.903 | 1416.732 |
| Compound 115 | [M + 5H]5+ | 1725.881 | 1725.885 |
| | [M + 6H]6+ | 1438.401 | 1438.405 |
| Compound 120 | [M + 5H]5+ | 1723.077 | 1722.890 |
| | [M + 6H]6+ | 1435.903 | 1435.910 |
| Compound 121 | [M + 5H]5+ | 1723.077 | 1722.890 |
| | [M + 6H]6+ | 1435.903 | 1435.910 |
| Compound 122 | [M + 5H]5+ | 1714.270 | 1714.282 |
| | [M + 6H]6+ | 1428.731 | 1428.736 |
| Compound 123 | [M + 5H]5+ | 1714.287 | 1714.085 |
| | [M + 6H]6+ | 1428.731 | 1428.572 |
| Compound 124 | [M + 5H]5+ | 1726.068 | 1726.082 |
| | [M + 6H]6+ | 1438.557 | 1438.569 |
| Compound 125 | [M + 5H]5+ | 1734.292 | 1734.103 |
| | [M + 6H]6+ | 1445.255 | 1445.253 |
| Compound 126 | [M + 5H]5+ | 1702.867 | 1702.877 |
| | [M + 6H]6+ | 1419.232 | 1419.232 |
| Compound 127 | [M + 5H]5+ | 1702.867 | 1702.877 |
| | [M + 6H]6+ | 1419.232 | 1419.232 |
| Compound 128 | [M + 5H]5+ | 1722.686 | 1722.698 |
| | [M + 6H]6+ | 1435.748 | 1435.750 |
| Compound 129 | [M + 5H]5+ | 1725.286 | 1725.297 |
| | [M + 6H]6+ | 1437.905 | 1437.916 |
| Compound 130 | [M + 5H]5+ | 1572.809 | 1572.817 |
| | [M + 6H]6+ | 1310.841 | 1310.849 |
| Compound 131 | [M + 5H]5+ | 1581.620 | 1581.622 |
| | [M + 6H]6+ | 1318.171 | 1318.186 |
| Compound 132 | [M + 5H]5+ | 1732.298 | 1732.302 |
| | [M + 6H]6+ | 1443.747 | 1443.753 |
| Compound 133 | [M + 5H]5+ | 1732.485 | 1732.499 |
| | [M + 6H]6+ | 1443.918 | 1443.917 |
| Compound 134 | [M + 5H]5+ | 1581.815 | 1581.819 |
| | [M + 6H]6+ | 1318.513 | 1318.350 |
| Compound 135 | [M + 5H]5+ | 1725.677 | 1725.693 |
| | [M + 6H]6+ | 1438.401 | 1438.246 |
| Compound 136 | [M + 5H]5+ | 1737.481 | 1737.294 |
| | [M + 6H]6+ | 1447.902 | 1447.913 |
| Compound 137 | [M + 5H]5+ | 1730.884 | 1730.896 |
| | [M + 6H]6+ | 1442.580 | 1442.581 |
| Compound 138 | [M + 5H]5+ | 1729.080 | 1728.892 |
| | [M + 6H]6+ | 1440.902 | 1440.911 |
| Compound 139 | [M + 5H]5+ | 1731.293 | 1731.299 |
| | [M + 6H]6+ | 1442.907 | 1442.917 |
| Compound 140 | [M + 5H]5+ | 1615.824 | 1615.835 |
| | [M + 6H]6+ | 1346.693 | 1346.697 |
| Compound 141 | [M + 5H]5+ | 1608.428 | 1608.230 |
| | [M + 6H]6+ | 1340.347 | 1340.359 |
| Compound 142 | [M + 5H]5+ | 1705.080 | 1705.088 |
| | [M + 6H]6+ | 1421.068 | 1421.074 |
| Compound 143 | [M + 5H]5+ | 1734.087 | 1734.103 |
| | [M + 6H]6+ | 1445.240 | 1445.253 |
| Compound 144 | [M + 5H]5+ | 1721.276 | 1721.287 |
| | [M + 6H]6+ | 1434.570 | 1434.574 |
| Compound 145 | [M + 5H]5+ | 1709.867 | 1709.882 |
| | [M + 6H]6+ | 1425.067 | 1425.070 |
| Compound 146 | [M + 5H]5+ | 1737.293 | 1737.294 |
| | [M + 6H]6+ | 1448.073 | 1447.913 |
| Compound 147 | [M + 5H]5+ | 1734.479 | 1734.491 |
| | [M + 6H]6+ | 1445.567 | 1445.577 |
| Compound 148 | [M + 5H]5+ | 1723.077 | 1723.087 |
| | [M + 6H]6+ | 1436.229 | 1436.074 |
| Compound 149 | [M + 5H]5+ | 1734.479 | 1734.491 |
| | [M + 6H]6+ | 1445.567 | 1445.577 |
| Compound 150 | [M + 5H]5+ | 1729.284 | 1729.288 |
| | [M + 6H]6+ | 1441.399 | 1441.241 |
| Compound 151 | [M + 5H]5+ | 1737.481 | 1737.294 |
| | [M + 6H]6+ | 1447.902 | 1447.913 |
| Compound 152 | [M + 5H]5+ | 1723.077 | 1723.087 |
| | [M + 6H]6+ | 1436.074 | 1436.074 |
| Compound 153 | [M + 5H]5+ | 1734.479 | 1734.491 |
| | [M + 6H]6+ | 1445.567 | 1445.577 |
| Compound 154 | [M + 5H]5+ | 1729.284 | 1729.288 |
| | [M + 6H]6+ | 1441.244 | 1441.241 |
| Compound 155 | [M + 5H]5+ | 1746.278 | 1746.092 |
| | [M + 6H]6+ | 1455.231 | 1455.245 |
| Compound 156 | [M + 5H]5+ | 1734.479 | 1734.491 |
| | [M + 6H]6+ | 1445.567 | 1445.577 |
| Compound 157 | [M + 5H]5+ | 1745.885 | 1745.895 |
| | [M + 6H]6+ | 1455.075 | 1455.081 |
| Compound 158 | [M + 5H]5+ | 1581.620 | 1581.622 |
| | [M + 6H]6+ | 1318.350 | 1318.186 |
| Compound 159 | [M + 5H]5+ | 1711.475 | 1711.486 |
| | [M + 6H]6+ | 1426.396 | 1426.406 |
| Compound 160 | [M + 5H]5+ | 1774.701 | 1774.507 |
| | [M + 6H]6+ | 1478.920 | 1478.924 |
| Compound 161 | [M + 5H]5+ | 1774.494 | 1774.507 |
| | [M + 6H]6+ | 1478.920 | 1478.924 |
| Compound 162 | [M + 5H]5+ | 1726.068 | 1726.082 |
| | [M + 6H]6+ | 1438.557 | 1438.569 |
| Compound 163 | [M + 5H]5+ | 1724.470 | 1724.478 |
| | [M + 6H]6+ | 1437.222 | 1437.233 |
| Compound 164 | [M + 5H]5+ | 1726.272 | 1726.279 |
| | [M + 6H]6+ | 1438.728 | 1438.733 |
| Compound 165 | [M + 5H]5+ | 1740.673 | 1740.486 |
| | [M + 6H]6+ | 1450.567 | 1450.573 |
| Compound 166 | [M + 5H]5+ | 1721.480 | 1721.484 |
| | [M + 6H]6+ | 1434.895 | 1434.738 |
| Compound 167 | [M + 5H]5+ | 1735.673 | 1735.691 |
| | [M + 6H]6+ | 1446.563 | 1446.577 |
| Compound 168 | [M + 5H]5+ | 1737.481 | 1737.491 |
| | [M + 6H]6+ | 1448.073 | 1448.077 |
| Compound 169 | [M + 5H]5+ | 1701.263 | 1701.274 |
| | [M + 6H]6+ | 1417.891 | 1417.896 |
| Compound 170 | [M + 5H]5+ | 1701.263 | 1701.077 |
| | [M + 6H]6+ | 1417.721 | 1417.732 |
| Compound 171 | [M + 5H]5+ | 1701.263 | 1701.274 |
| | [M + 6H]6+ | 1417.891 | 1417.896 |
| Compound 172 | [M + 5H]5+ | 1704.455 | 1704.466 |
| | [M + 6H]6+ | 1420.543 | 1420.556 |
| Compound 173 | [M + 5H]5+ | 1715.864 | 1715.870 |
| | [M + 6H]6+ | 1430.046 | 1430.060 |
| Compound 174 | [M + 5H]5+ | 1713.457 | 1713.463 |
| | [M + 6H]6+ | 1428.051 | 1428.054 |
| Compound 175 | [M + 5H]5+ | 1727.854 | 1727.670 |
| | [M + 6H]6+ | 1439.892 | 1439.893 |
| Compound 176 | [M + 5H]5+ | 1580.009 | 1580.019 |
| | [M + 6H]6+ | 1317.013 | 1316.850 |
| Compound 177 | [M + 5H]5+ | 1596.026 | 1596.026 |
| | [M + 6H]6+ | 1330.176 | 1330.190 |
| Compound 178 | [M + 5H]5+ | 1594.424 | 1594.423 |
| | [M + 6H]6+ | 1328.848 | 1328.854 |
| Compound 179 | [M + 5H]5+ | 1584.812 | 1584.814 |
| | [M + 6H]6+ | 1320.833 | 1320.846 |
| Compound 180 | [M + 5H]5+ | 1583.004 | 1583.014 |
| | [M + 6H]6+ | 1319.509 | 1319.346 |
| Compound 181 | [M + 5H]5+ | 1594.228 | 1594.226 |
| | [M + 6H]6+ | 1328.684 | 1328.690 |
| Compound 182 | [M + 5H]5+ | 1597.416 | 1597.418 |
| | [M + 6H]6+ | 1331.341 | 1331.350 |
| Compound 183 | [M + 5H]5+ | 1571.203 | 1571.214 |
| | [M + 6H]6+ | 1309.507 | 1309.513 |
| Compound 184 | [M + 5H]5+ | 1576.009 | 1576.009 |
| | [M + 6H]6+ | 1313.495 | 1313.508 |
| Compound 185 | [M + 5H]5+ | 1590.406 | 1590.413 |
| | [M + 6H]6+ | 1325.508 | 1325.512 |
| Compound 186 | [M + 5H]5+ | 1574.401 | 1574.405 |
| | [M + 6H]6+ | 1312.160 | 1312.172 |
| Compound 187 | [M + 5H]5+ | 1585.610 | 1585.618 |
| | [M + 6H]6+ | 1321.502 | 1321.516 |
| Compound 188 | [M + 5H]5+ | 1588.806 | 1588.810 |
| | [M + 6H]6+ | 1324.167 | 1324.176 |
| Compound 189 | [M + 5H]5+ | 1723.485 | 1723.284 |
| | [M + 6H]6+ | 1436.229 | 1436.238 |
| Compound 190 | [M + 5H]5+ | 1721.276 | 1721.287 |
| | [M + 6H]6+ | 1434.570 | 1434.574 |
| Compound 191 | [M + 5H]5+ | 1721.480 | 1721.484 |
| | [M + 6H]6+ | 1434.895 | 1434.738 |
| Compound 192 | [M + 5H]5+ | 1730.271 | 1730.085 |
| | [M + 6H]6+ | 1441.896 | 1441.906 |
| Compound 193 | [M + 5H]5+ | 1588.806 | 1588.817 |
| | [M + 6H]6+ | 1324.182 | 1324.182 |
| Compound 194 | [M + 5H]5+ | 1585.806 | 1585.815 |
| | [M + 6H]6+ | 1321.845 | 1321.680 |
| Compound 195 | [M + 5H]5+ | 1727.276 | 1727.279 |
| | [M + 6H]6+ | 1439.566 | 1439.567 |
| Compound 196 | [M + 5H]5+ | 1734.292 | 1734.299 |
| | [M + 6H]6+ | 1445.411 | 1445.417 |
| Compound 197 | [M + 5H]5+ | 1737.481 | 1737.294 |
| | [M + 6H]6+ | 1447.902 | 1447.913 |
| Compound 210 | [M + 5H]5+ | 1577.390 | 1577.194 |
| | [M + 6H]6+ | 1314.489 | 1314.496 |
| Compound 211 | [M + 5H]5+ | 1588.594 | 1588.603 |
| | [M + 6H]6+ | 1324.167 | 1324.004 |
| Compound 212 | [M + 5H]5+ | 1585.790 | 1585.800 |
| | [M + 6H]6+ | 1321.666 | 1321.668 |
| Compound 213 | [M + 5H]5+ | 1574.384 | 1574.395 |
| | [M + 6H]6+ | 1312.160 | 1312.164 |
| Compound 214 | [M + 5H]5+ | 1585.790 | 1585.800 |
| | [M + 6H]6+ | 1321.830 | 1321.668 |
| Compound 215 | [M + 5H]5+ | 1580.594 | 1580.597 |
| | [M + 6H]6+ | 1317.325 | 1317.332 |
| Compound 216 | [M + 5H]5+ | 1588.594 | 1588.603 |
| | [M + 6H]6+ | 1324.167 | 1324.004 |
| Compound 217 | [M + 5H]5+ | 1574.384 | 1574.395 |
| | [M + 6H]6+ | 1312.160 | 1312.164 |
| Compound 218 | [M + 5H]5+ | 1585.790 | 1585.800 |
| | [M + 6H]6+ | 1321.666 | 1321.668 |
| Compound 219 | [M + 5H]5+ | 1580.594 | 1580.597 |
| | [M + 6H]6+ | 1317.325 | 1317.332 |
| Compound 220 | [M + 5H]5+ | 1574.189 | 1574.199 |
| | [M + 6H]6+ | 1311.997 | 1312.000 |
| Compound 221 | [M + 5H]5+ | 1585.610 | 1585.411 |
| | [M + 6H]6+ | 1321.339 | 1321.344 |

### Example 4: Human GLP-1-, GIP-, and Amylin-Receptors In Vitro Potency Assays (High Throughput Assay)

### GLP-1 Receptor Assay

To determine the ability of compounds to activate or agonize the GLP-1 receptor, in vitro potency assays on Baby Hamster Kidney (BHK) cells expressing the human GLP-1 receptor (hGLP-1R) were performed as described below. To assess how the activation of the receptors is potentially influenced by the presence of human serum albumin (HSA), the in vitro assays were performed in the absence of HSA and presence of 1% (w/v) HSA. Unless stated otherwise the reference to the “GLP-1 receptor assay as described in Example 4” throughout the specification shall refer to the described herein assay procedure method A (hGLP-1R assay) in the absence of HSA.

### Assay Principle

Activation of the human GLP-1 receptor leads to increased intra-cellular concentrations of cyclic AMP (cAMP) and the consequent transcription activation from promoters containing multiple copies of the cAMP response element (CRE). It is thus possible to measure GLP-1 receptor activity using a CRE-luciferase reporter gene introduced into Baby Hamster Kidney (BHK) cells co-expressing the human GLP-1 receptor.

### Cells and Assay Reagents

Cell stocks were prepared by culturing of a cell line stably expressing the human GLP-1 receptor and the CRE responsive luciferase (CRE-Luc) reporter gene (BHK 467-12A KZ-10 prepared according to methods known to the person skilled in the art) in growth medium consisting of DMEM (Gibco, 61965-026) supplemented with 10% FBS (Gibco, 16140-071 or 10100-147), 1% Penicillin-Streptomycin (Gibco, 15140-122), 1 mM Na-Pyruvate (Gibco, 11360-039), 1 mg/mL G418 (Gibco, 10131-027) and 240 nM Methotrexate (Pfizer, 15936). Cells at approximately 80-90% confluence were washed once in PBS (Gibco 14190-094) and loosened from the cell flasks with Versene (Gibco, 15040-033). After centrifugation, the cell pellet was resuspended and diluted to approximately 1.5×10⁶ (1.5E+6) cells/mL in Recovery™ Cell Culture Freezing Medium (Gibco, 12648-010) or in medium consisting of DMEM (Gibco, 61965-026) supplemented with 20% FBS (Gibco, 16140-071 or 10100-147), 1% Penicillin-Streptomycin (Gibco, 15140-122), 1 mM Na-Pyruvate (Gibco, 11360-039), 1 mg/mL G418 (Gibco, 10131-027), 240 nM Methotrexate (Pfizer, 15936) and 10% DMSO (Sigma, D2650). Cells were aliquoted and stored at −180° C. until use.

The assay buffer consisted of DMEM without phenol red (Gibco, 11880-028) supplemented with 1× GlutaMAX (Gibco, 35050-038), 10 mM HEPES (Gibco, 15630-056), 1% (w/v) ovalbumin (Sigma, A5503) and 0.1% (v/v) Pluronic F-68 (Gibco, 24040-032) either with HSA or without HSA (Sigma, A9511).

Procedure—Method A (hGLP-1R Assay)

To perform the assay, serial dilutions (10-fold dilutions, 8 concentrations per compound) of reference compounds and GLP-1-/GIP-/amylin-receptor tri-agonists were performed in assay buffer without HSA in a 96-well plate. Frozen stocks of hGLP-1R BHK CRE-Luc cells were thawed in a 37° C. water bath, washed once in PBS (Gibco 14190-094) and diluted to 1.5×10⁶ (1.5E+6) cells/mL in assay buffer with or without 2% (w/v) HSA (Sigma, A9511). For each dilution, 50 μL aliquots of reference compounds or GLP-1-/GIP-/amylin-receptor tri-agonists were transferred to two 96-well assay plates (ThermoFisher, 237105) to which 50 μL of the cell suspension with or without 2% (w/v) HSA was added (5.0×10³ (5.0E+3) cells/well). The assay plates were incubated for 3 hours at 37° C. in 5% CO₂, left at room temperature for 5 minutes after which 100 μL steadylite Plus™ (PerkinElmer/Revvity, 6066759) was added to each well. Plates were sealed and incubated at room temperature with gentle shaking for 30 minutes while protected from light. Luminescence was detected on a luminescence plate reader e.g., a Synergy 2 (BioTek). The EC₅₀-values [pM] were calculated by non-linear curve fitting applying a four-parameter logistic model (Hill slope=1) using GraphPad Prism (GraphPad Software, Boston, MA, USA) or by means of TIBCO Enterprise Runtime for R (TIBCO Software, Palo Alto, CA, USA).

Procedure—Method B (hGLP-1R Assay)

To perform the assay, serial dilutions (7-fold dilutions, 7 concentrations per compound and one well containing only assay buffer) of reference compounds and GLP-1-/GIP-/amylin-receptor tri-agonists were performed in assay buffer in a 96-well plate. Serial dilutions were transferred to a 384-well assay plate (PerkinElmer/Revvity, 6007688) and mixed with an equal volume (10 μL) of assay buffer without or with 3% (w/v) HSA (Sigma, A-9511). Frozen stocks of hGLP-1R BHK Cre-Luc cells were thawed in a 37° C. water bath, washed once in PBS (Gibco 14190-094), diluted to 1.5×10⁵ (1.5E+5) cells/mL in assay buffer (without HSA) and added (10 μL) to each well of the 384-well assay plate. After a short centrifugation, the assay plates were incubated for 3 hours at 37° C. in 5% CO2 and let to equilibrated at room temperature for 10 minutes before the addition of 30 μL steadylite Plus™ PerkinElmer/Revvity, 6066759) per well. Plates were sealed and incubated at room temperature with gentle shaking for 30 minutes while protected from light. Luminescence was detected on a luminescence plate reader e.g. a Synergy 2 (BioTek). The EC₅₀-values [pM] were calculated by non-linear curve fitting applying a four-parameter logistic model (Hill slope=1) using GraphPad Prism (GraphPad Software, Boston, MA, USA) or by means of TIBCO Enterprise Runtime for R (TIBCO Software, Palo Alto, CA, USA).

### GIP Receptor Assay

To determine the ability of compounds to activate or agonize the GIP receptor, in vitro potency assays on Baby Hamster Kidney (BHK) cells expressing the human GIP receptor (hGIPR) were performed as described below. To assess how the activation of the receptors is potentially influenced by the presence of human serum albumin (HSA), the in vitro assays were performed in the absence of HSA and presence of 1% (w/v) HSA. Unless stated otherwise the reference to the “GIP receptor assay as described in Example 4” throughout the specification shall refer to the described herein assay procedure method A (hGIPR assay) in the absence of HSA.

### Assay Principle

Activation of the human GIP receptor leads to increased intra-cellular concentrations of cyclic AMP (cAMP) and the consequent transcription activation from promoters containing multiple copies of the cAMP response element (CRE). It is thus possible to measure GIP receptor activity using a CRE-luciferase reporter gene introduced into Baby Hamster Kidney (BHK) cells co-expressing the human GIP receptor.

### Cells and Assay Reagents

Cell stocks were prepared by culturing of a cell line stably expressing the human GIP receptor and containing the CRE responsive luciferase (CRE-Luc) reporter gene (hGIPR BHK Cre-Luc2p clone #5 prepared according to methods known to the person skilled in the art) at 5% CO₂ and 37° C. in growth medium consisting of in DMEM (Gibco, 61965-026) supplemented with 10% fetal calf serum (Gibco, 16140-071 or 10100-147), 0.5 mg/ml G418 (Gibco, 10131-027), 1% Penicillin-Streptomycin (Gibco, 15140-122) and 0.3 mg/ml Hygromycin B (Invitrogen, 10687010). Cells at about 80-90% confluency were washed once with PBS (Gibco 14190-094) and detached from the cell flasks with Versene (Gibco, 15040-066). After centrifugation, the cells were counted, resuspended and diluted to approximately 1.5-3.0×10⁶ (1.5E+6 to 3.0E+6) cells/mL in Recovery Cell Culture Freezing Medium (Gibco, 12648-010) and stored at −180° C. in suitable aliquots until use.

The assay buffer consisted of DMEM without phenol red (Gibco, 11880-028) supplemented with 1× GlutaMAX (Gibco, 35050-038), 10 mM HEPES (Gibco, 15630-056), 1% (w/v) ovalbumin (Sigma, A5503) and 0.1% (v/v) Pluronic F-68 (Gibco, 24040-032) either with HSA or without HSA (Sigma, A9511).

Procedure—Method A (hGIPR Assay)

On the day before the assay hGIPR BHK Cre-Luc cells were thawed and plated out in 96 well Culture Plates (PerkinElmer/Revvity, 6005680) at 5.0×10³ (5.0E+3) cells/well in growth medium as described above. The plates were then incubated for 21-23 hours at 37° C. with 5% CO₂. On the day of the assay GLP-1-/GIP-/amylin-receptor tri-agonists or reference compounds were diluted in assay buffer using a 7-point, 10-fold titration plus a blank (absence of compound). Each compound was diluted and tested in duplicates in each experiment. In the plates containing the cells the growth medium was removed, and cells were washed twice with 100 μL PBS (Gibco 14190-094). 50 μL of the test compound dilutions were added to the plates containing cells preceded by 50 μL assay buffer with or without 2% (w/v) HSA (Sigma, A9511). The cell plates were incubated for 3 hours in a 5% CO₂ incubator at 37° C. The plates were then transferred to room temperature followed by addition of 100 μL steadylite Plus™ (PerkinElmer/Revvity, 6066759). The plates were sealed and shaken for 30 minutes at room temperature while protected from light. Finally, luminescence (as an indicator of receptor activation) was measured on a Mithras reader (Berthold Technologies, DE). The EC₅₀-values [pM] were calculated by non-linear curve fitting applying a four-parameter logistic model (Hill slope=“shared value for all datasets”) using GraphPad Prism (GraphPad Software, Boston, MA, USA).

Procedure—Method B (hGIPR Assay)

To perform the assay, serial dilutions (7-fold dilutions, 7 concentrations per compound and one well containing only assay buffer) of reference compounds and GLP-1-/GIP-/amylin-receptor tri-agonists were prepared in assay buffer in a 96-well plate. Serial dilutions were transferred to a 384-well assay plate (PerkinElmer/Revvity, 6007688) and mixed with an equal volume (10 μL) of assay buffer without or with 3% HSA (Sigma, A-9511). Frozen stocks of hGIPR BHK Cre-Luc cells were thawed in a 37° C. water bath, washed once in PBS (Gibco 14190-094), diluted to 1.5×10⁵ (1.5E+5) cells/mL in assay buffer (without HSA) and added (10 μL) to each well of the 384-well assay plate. After a short centrifugation, the assay plates were incubated for 3 hours at 37° C. in 5% CO2 and let to equilibrated at room temperature for 10 minutes before the addition of 30 μL steadylite Plus™ (PerkinElmer/Revvity, 6066759) per well. Plates were sealed and incubated at room temperature with gentle shaking for 30 minutes while protected from light. Luminescence was detected on a luminescence plate reader e.g. a Synergy 2 (BioTek). The EC₅₀-values [pM] were calculated by non-linear curve fitting applying a four-parameter logistic model (Hill slope=1) using GraphPad Prism (GraphPad Software, Boston, MA, USA) or by means of TIBCO Enterprise Runtime for R (TIBCO Software, Palo Alto, CA, USA).

### Amylin Receptor Assay

To determine the ability of compounds to activate or agonize the amylin receptor, in vitro potency assays on Baby Hamster Kidney (BHK) cells expressing the human amylin receptor (hAMYR3) were performed as described below. To assess how the activation of the receptors is potentially influenced by the presence of human serum albumin (HSA), the in vitro assays were performed in the absence of HSA and presence of 1% (w/v) HSA. Unless stated otherwise the reference to the “amylin receptor assay as described in Example 4” throughout the specification shall refer to the described herein assay procedure method A (hAMYR3 assay) in the absence of HSA.

### Assay Principle

Activation of the human amylin 3 receptor leads to increased intra-cellular concentrations of cAMP and the consequent transcription activation from promoters containing multiple copies of the cAMP response element (CRE). It is thus possible to measure hAMYR3 activity using a CRE-luciferase reporter gene introduced into Baby Hamster Kidney (BHK) cells co-expressing the hAMYR3.

### Cells and Assay Reagents

A BHK cell line was transfected to stably express the human calcitonin receptor₍ₐ₎ and a CRE-responsive luciferase (CRE-Luc) reporter gene according to methods known to the person skilled in the art (Hollex-1 cell line, obtained from Zymogenetics described in U.S. Pat. No. 5,622,839). The cell line was further transfected with human receptor modifying protein 3 (RAMP3) using standard methods. This turns the human calcitonin receptor into a human amylin-3(a) receptor (hAMYR3).

Cells stocks were prepared by culturing of the hAMYR3 BHK Cre-Luc cell line in growth medium consisting of DMEM (Gibco, 31966-021) supplemented with 10% FBS (Gibco, 16140-071 or 10100-147), 1% Penicillin-Streptomycin (Gibco, 15140-122), 0.5 mg/mL Geneticin (Gibco, 10131-027), 0.4 mg/mL Hygromycin (Invitrogen, 10687010) and 250 nM Methotrexate (Sigma, A6770). Cells at approximately 80-90% confluence were washed once with PBS (Gibco 14190-094) and loosened from the cell flasks with Versene (Gibco, 15040-033) or TrypLE™ (Gibco, 12605-010). After centrifugation, the cell pellet was resuspended and diluted to approximately 2.5-4.0×10⁶ (2.5E+6 to 4.0E+6) cells/mL in Recovery™ Cell Culture Freezing Medium (Gibco, 12648-010). Cells were aliquoted and stored at −180° C. until use.

The assay buffer consisted of DMEM without phenol red (Gibco, 11880-028) supplemented with 1× GlutaMAX (Gibco, 35050-038), 10 mM HEPES (Gibco, 15630-056) and 1% (w/v) ovalbumin (Sigma, A5503) either with or without 0.1% (v/v) Pluronic F-68 (Gibco, 24040-032) and either with or without HSA (Sigma, A9511).

Procedure—Method A (hAMYR3 Assay)

To perform the assay, BHK hAMYR3/CRE-Luc cells were thawed, washed once in PBS (Gibco 14190-094), and seeded in 40 μL growth medium in a white 384-well culture plate (PerkinElmer/Revvity, 6007688) at a cell density of 4.0×10³ (4.0E+3) cells/well on the day before the experiment. The plate was incubated over night at 37° C. in 5% CO₂. On the day of the assay, cells were washed once in assay PBS (Gibco 14190-094). Serial dilutions (7-fold dilutions, 7 concentrations pr. compound and one well containing only assay buffer) of reference compounds and GLP-1-/GIP-/amylin-receptor tri-agonists were performed in assay buffer with or without 1% (w/v)HSA (Sigma, A9511) in 96-well plates and 30 μL of each concentration added to the 384-well assay plate with cells. The assay plate was incubated for 3 hours at 37° C. in 5% CO₂ after which 30 μL steadylite Plus™ (PerkinElmer/Revvity, 6066759) was added to each well. The assay plate was sealed, incubated at room temperature with gentle shaking for 5 minutes followed by 30 minutes incubation without shaking while protected from light. Luminescence was detected on a luminescence plate reader e.g., a Synergy 2 (BioTek). The EC₅₀-values [pM] were calculated by non-linear curve fitting applying a four-parameter logistic model (Hill slope=1.5, shared bottom response within each plate) using GraphPad Prism (GraphPad Software, Boston, MA, USA) or by means of TIBCO Enterprise Runtime for R (TIBCO Software, Palo Alto, CA, USA).

Procedure—Method B (hAMYR3)

To perform the assay, hAMYR3 BHK Cre-Luc cells were thawed, washed once in PBS (Gibco 14190-094) and seeded in 40 μL growth medium in a white 384-well culture plate (PerkinElmer/Revvity, 6007688) at a cell density of 4.0×10³ (4.0E+3) cells/well the day before the experiment. The plate was incubated over night at 37° C. in 5% CO2. On the day of the assay, serial dilutions (7-fold dilutions, 7 concentrations per compound and one well containing only assay buffer) of reference compounds and GLP-1-/GIP-/amylin-receptor tri-agonists were prepared in assay buffer in 96-well plates. Serial dilutions were then mixed in a new 96-well plate with equal volume (1:1:1 ratio) of assay buffer and either assay buffer without or with 3% HSA (Sigma, A-9511). Twenty microliters of the solution mix were transferred to the cells which previously were washed once with PBS (Gibco 14190-094). After a short centrifugation, the assay plates were incubated for 3 hours at 37° C. in 5% CO₂ and let to equilibrated at room temperature for 10 minutes before the addition of 30 μL steadylite Plus™ PerkinElmer/Revvity, 6066759) per well. Plates were sealed and incubated at room temperature with gentle shaking for 30 minutes while protected from light. Luminescence was detected on a luminescence plate reader e.g., a Synergy 2 (BioTek). The EC₅₀-values [pM] were calculated by non-linear curve fitting applying a four-parameter logistic model (Hill slope=1.5, shared bottom response within each plate) using GraphPad Prism (GraphPad Software, Boston, MA, USA) or by means of TIBCO Enterprise Runtime for R (TIBCO Software, Palo Alto, CA, USA).

TABLE 7a
In vitro activity data for reference compounds
on hGLP-1R, hGIPR, and hAMYR3 measured (according
to methods A) in the absence of HSA

| Reference | hGLP-1R | hGIPR Potency | hAMYR3 Potency, |
|---|---|---|---|
| compound | Potency, | no HSA, | no HSA, |
| number | no HSA, EC₅₀ [pM] | EC₅₀ [pM] | EC₅₀ [pM] |
| 1 | 1516.05 | 29.63 | 5.29 |
| 2 | 586.71 | 3.16 | 20.27 |
| 3 | 267.92 | 2.31 | 4.49 |
| 4 | nd | 1.01 | nd |
| 5 | 43.0 | 0.91 | nd |
| 6 | nd | nd | 1.47 |
| 7 | 2.67 | nd | nd |

TABLE 7b
In vitro activity data for reference compounds
on hGLP-1R, hGIPR, and hAMYR3 measured (according
to methods B) in the absence of HSA

| Reference | hGLP-1R | hGIPR Potency | hAMYR3 Potency, |
|---|---|---|---|
| compound | Potency, | no HSA, | no HSA, |
| number | no HSA, EC₅₀ [pM] | EC₅₀ [pM] | EC₅₀ [pM] |
| 4 | nd | 2.41 | nd |
| 6 | nd | nd | 2.47 |
| 7 | 4.97 | nd | nd |

The results in Table 7a and Table 7b show that the reference compounds 4 to 7 are agonists or co-agonists on one or two of the GLP-1 receptor, GIP receptor, and amylin receptor (hAMYR3).

According to Table 7a, the activity data for reference compound 2 show that linking the C-terminus of a potent GLP-1/GIP co-agonist (tirzepatide), via a peptide linker, to the N-terminus of a potent amylin receptor agonist (cagrilintide) does not result in a compound that is equally potent on these three receptors and that can necessarily function as a GLP-1-/GIP-/amylin-receptor tri-agonist (i.e., a compound according to the invention). A comparison of the reference compound 2 with the reference compound 5 (tirzepatide) and reference compound 6 (cagrilintide) illustrates this point. The reference compound 2 significantly loses potency on the GLP-1 receptor and show further some loss of potency on the amylin receptor, when compared to the original compounds, reference compounds 5 (tirzepatide) and 6 (cagrilintide).

The reference compounds 1 to 3 show a functional activation of all three receptors but have all an impaired potency on the GLP-1 receptor, and therefore are unbalanced tri-agonists having a potency ratio (A/B) between 116 and 287.

TABLE 8a
In vitro activity data for the synthesized compound
of the invention on hGLP-1R, hGIPR, and hAMYR3 measured
(according to methods A) in the absence of HSA

| | hGLP-1R | hGIPR | hAMYR3 |
|---|---|---|---|
| | Potency, | Potency, | Potency, |
| Compound | no HSA, | no HSA, | no HSA, |
| number | EC₅₀ [pM] | EC₅₀ [pM] | EC₅₀ [pM] |
| 10 | 13.83 | 2.11 | 6.8 |
| 11 | 10.43 | 1.69 | 12.79 |
| 12 | 0.8 | 2.62 | 6.23 |
| 13 | 37.92 | 2.4 | 2.26 |
| 14 | 2.05 | 5.84 | 2.21 |
| 15 | 1.41 | 3.2 | 5.01 |
| 16 | 2.74 | 4.27 | 5.7 |
| 17 | 2.72 | 5.04 | 5.13 |
| 18 | 52.83 | 7.25 | 3.93 |
| 19 | 57.91 | 9.44 | 4.24 |
| 20 | 40.64 | 4.82 | 3.65 |
| 21 | 41.18 | 8.63 | 11.98 |
| 22 | 11.33 | 35.77 | 10.92 |
| 23 | 15.35 | 1.2 | 5.17 |
| 24 | 51.49 | 2.34 | 4.24 |
| 25 | 2.87 | 4.27 | 3.22 |
| 26 | 20.71 | 1.75 | 11.8 |
| 27 | 26.24 | 2.11 | 3.82 |
| 28 | 95.98 | 3.5 | 2.28 |
| 29 | 27.08 | 2.62 | 3.09 |
| 30 | 6.37 | 6.8 | 2.28 |
| 31 | 47.78 | 4.73 | 2.69 |
| 32 | 27.55 | 8.09 | 10.09 |
| 33 | 32.13 | 3.11 | 10.26 |
| 34 | 21.71 | 1.7 | 6.81 |
| 35 | 23.26 | 1.97 | 4.21 |
| 36 | 3.19 | 1.96 | 3.84 |
| 37 | 3.94 | 2.34 | 2.98 |
| 38 | 11.97 | 1.37 | 3.63 |
| 39 | 23.74 | 1.62 | 5.75 |
| 40 | 23.28 | 1.63 | 5.49 |
| 41 | 40.79 | 5.02 | 6.72 |
| 42 | 61.34 | 8.01 | 3.42 |
| 43 | 4.31 | 4.38 | 3.13 |
| 44 | 3.68 | 6.3 | 3.8 |
| 45 | 4.78 | 2.63 | 4.69 |
| 46 | 4.59 | 5.42 | 4.41 |
| 47 | 3.15 | 2.02 | 2.57 |
| 48 | 64.76 | 7.02 | 3.2 |
| 49 | 33.26 | 4.36 | 1.85 |
| 50 | 16.18 | 2.36 | 3.2 |
| 51 | 7.68 | 8.37 | 6.19 |
| 52 | 11.03 | 1.83 | 2.85 |
| 53 | 23.34 | 3.25 | 3.22 |
| 54 | 28.83 | 5.56 | 3.02 |
| 55 | 23.02 | 3.0 | 2.22 |
| 56 | 18.22 | 2.65 | 2.36 |
| 57 | 12.75 | 1.98 | 3.0 |
| 58 | 12.73 | 1.88 | 2.91 |
| 59 | 50.63 | 7.72 | 3.85 |
| 60 | 94.38 | 6.06 | 3.85 |
| 61 | 2.79 | 1.3 | 2.97 |
| 62 | 4.13 | 2.26 | 3.35 |
| 63 | 4.77 | 1.6 | 2.86 |
| 64 | 3.71 | 2.03 | 1.91 |
| 65 | 4.86 | 1.89 | 2.84 |
| 66 | 5.25 | 2.39 | 2.32 |
| 67 | 15.4 | 4.28 | 3.66 |
| 68 | 12.08 | 1.83 | 2.57 |
| 69 | 3.45 | 1.9 | 3.43 |
| 70 | 3.48 | 5.89 | 4.53 |
| 71 | 3.34 | 2.77 | 8.66 |
| 72 | 13.05 | 2.12 | 6.57 |
| 73 | 4.55 | 4.98 | 1.99 |
| 74 | 29.76 | 2.51 | 2.73 |
| 75 | 3.66 | 2.65 | 2.49 |
| 76 | 19.9 | 2.82 | 3.11 |
| 77 | 19.69 | 2.53 | 3.13 |
| 78 | 4.03 | 3.49 | 1.83 |
| 79 | 38.68 | 4.7 | 3.53 |
| 80 | 24.76 | 4.01 | 3.23 |
| 81 | 16.0 | 3.31 | 4.07 |
| 82 | 3.45 | 3.7 | 3.26 |
| 83 | 2.3 | 2.92 | 2.9 |
| 84 | 3.31 | 3.0 | 4.41 |
| 85 | 3.52 | 2.71 | 4.4 |
| 86 | 6.69 | 5.11 | 3.31 |
| 87 | 8.49 | 7.26 | 4.99 |
| 88 | 4.12 | 5.68 | 3.7 |
| 89 | 3.86 | 6.47 | 3.12 |
| 90 | 4.16 | 5.4 | 5.45 |
| 91 | 3.79 | 3.21 | 2.26 |
| 92 | 2.55 | 3.12 | 2.64 |
| 93 | 4.11 | 2.54 | 2.23 |
| 94 | 16.08 | 2.9 | 3.91 |
| 95 | 17.85 | 3.13 | 3.76 |
| 96 | 16.83 | 3.62 | 3.73 |
| 97 | 16.06 | 2.3 | 2.81 |
| 98 | 13.26 | 2.58 | 2.3 |
| 99 | 16.73 | 2.09 | 2.82 |
| 100 | 5.85 | 3.95 | 3.05 |
| 101 | 3.24 | 1.87 | 3.08 |
| 102 | 10.97 | 2.24 | 3.26 |
| 103 | 5.24 | 3.14 | 2.42 |
| 104 | 4.16 | 3.57 | 1.49 |
| 105 | 12.57 | 3.08 | 2.88 |
| 106 | 3.33 | 4.8 | 2.07 |
| 107 | 4.1 | 3.87 | 3.07 |
| 108 | 4.57 | 4.01 | 2.48 |
| 109 | 68.94 | 7.42 | 3.65 |
| 110 | 107.07 | 8.01 | 3.27 |
| 111 | 15.88 | 3.66 | 3.33 |
| 112 | 4.37 | 2.42 | 3.65 |
| 113 | 14.43 | 2.02 | 2.53 |
| 114 | 43.29 | 4.29 | 2.27 |

TABLE 8b
In vitro activity data for the synthesized compound
of the invention on hGLP-1R, hGIPR, and hAMYR3 measured
(according to methods B) in the absence of HSA

| | hGLP-1R | hGIPR | hAMYR3 |
|---|---|---|---|
| | Potency, | Potency, | Potency, |
| Compound | no HSA, | no HSA, | no HSA, |
| number | EC₅₀ [pM] | EC₅₀ [pM] | EC₅₀ [pM] |
| 115 | 19.4 | 10.3 | 7.13 |
| 120 | 25.3 | 2.9 | 6.31 |
| 121 | 9.5 | 2.95 | 7.63 |
| 122 | 21.0 | 3.73 | 7.68 |
| 123 | 19.9 | 3.69 | 7.89 |
| 124 | 21.3 | 3.4 | 15.2 |
| 125 | 23.8 | 3.06 | 6.26 |
| 126 | 21.7 | 3.38 | 11. |
| 127 | 5.28 | 3.15 | 12.4 |
| 128 | 43.9 | 6.31 | 7.5 |
| 129 | 25. | 3.66 | 5.46 |
| 130 | 21.2 | 4.05 | 7.24 |
| 131 | 29.6 | 4.24 | 5.44 |
| 132 | 29.8 | 3.69 | 5.29 |
| 133 | 21.4 | 3.67 | 8.78 |
| 134 | 27.9 | 3.88 | 7.33 |
| 135 | 33.6 | 4.86 | 8.3 |
| 136 | 34.3 | 4.64 | 10.2 |
| 137 | 28.3 | 3.71 | 5.1 |
| 138 | 19.7 | 2.3 | 5.5 |
| 139 | 19.0 | 2.1 | 7.1 |
| 140 | 28.0 | 3.3 | 10.2 |
| 141 | 24.2 | 2.53 | 7.64 |
| 142 | 26.9 | 3.9 | 6.43 |
| 143 | 28.8 | 3.89 | 6.16 |
| 144 | 20.8 | 2.72 | 8.07 |
| 145 | 25.0 | 4.15 | 10.2 |
| 146 | 34.0 | 2.93 | 6.83 |
| 147 | 33.6 | 3.06 | 7.12 |
| 148 | 25.7 | 2.35 | 6.56 |
| 149 | 30.8 | 3.14 | 8.86 |
| 150 | 26.2 | 2.99 | 7.61 |
| 151 | 27.7 | 3.99 | 9.67 |
| 152 | 22.4 | 3.83 | 8.5 |
| 153 | 21.2 | 2.25 | 7.54 |
| 154 | 33.8 | 3.42 | 8.11 |
| 155 | 10.0 | 4.56 | 11.1 |
| 156 | 9.03 | 6.59 | 11.8 |
| 157 | 8.48 | 5.6 | 9.6 |
| 158 | 11.2 | 3.6 | 11.8 |
| 159 | 26.2 | 3.16 | 7.42 |
| 160 | 17.6 | 2.2 | 8.2 |
| 161 | 19.3 | 2.6 | 9.7 |
| 162 | 15.1 | 1.79 | 10.4 |
| 163 | 15.7 | 1.63 | 20.4 |
| 164 | 14.4 | 1.93 | 10.5 |
| 165 | 25.1 | 2.39 | 9.4 |
| 166 | 15.0 | 1.76 | 9.12 |
| 167 | 23.0 | 2.0 | 9.89 |
| 168 | 25.8 | 2.31 | 6.59 |
| 169 | 13.1 | 1.9 | 15.0 |
| 170 | 15.2 | 1.8 | 6.9 |
| 171 | 10.7 | 2.1 | 11.0 |
| 172 | 12.8 | 2.1 | 20.8 |
| 173 | 11.8 | 1.8 | 22.2 |
| 174 | 14.0 | 2.1 | 72.0 |
| 175 | 14.3 | 2.3 | 34.3 |
| 176 | 29.9 | 3.66 | 11.2 |
| 177 | 35.9 | 4.13 | 8.37 |
| 178 | 27.0 | 3.59 | 12.9 |
| 179 | 23.3 | 4.36 | 10.0 |
| 180 | 13.3 | 2.2 | 9.93 |
| 181 | 28.8 | 3.25 | 8.46 |
| 182 | 21.0 | 3.2 | 17.2 |
| 183 | 14.7 | 2.0 | 6.2 |
| 184 | 13.3 | 1.72 | 7.57 |
| 185 | 15.2 | 2.2 | 11.2 |
| 186 | 14.1 | 2.14 | 16.5 |
| 187 | 31.5 | 4.73 | 17.2 |
| 188 | 20.2 | 3.52 | 31.9 |
| 189 | 26.9 | 3.18 | 12.7 |
| 190 | 6.0 | 2.1 | 10.6 |
| 191 | 7.9 | 2.7 | 16.0 |
| 192 | 35.0 | 3.68 | 8.17 |
| 193 | 49.0 | 3.74 | 8.67 |
| 194 | 303.0 | 2.89 | 11.0 |
| 195 | 244.0 | 3.58 | 14.0 |
| 196 | 42.5 | 4.56 | 12.8 |
| 197 | 18.3 | 3.92 | 8.16 |
| 210 | 16.4 | 9.57 | 7.28 |
| 211 | 15.2 | 1.83 | 3.8 |
| 212 | 12.7 | 1.92 | 3.6 |
| 213 | 11.1 | 1.9 | 3.6 |
| 214 | 12.2 | 1.64 | 3.8 |
| 215 | 11.6 | 1.7 | 3.8 |
| 216 | 9.9 | 1.7 | 4.5 |
| 217 | 9.0 | 1.7 | 5.9 |
| 218 | 8.8 | 1.6 | 5.4 |
| 219 | 2.7 | 0.5 | 5.0 |
| 220 | 12.5 | 2.22 | 7.05 |
| 221 | 17.0 | 2.39 | 6.76 |

The results in Table 8a/b show that the compounds of the present invention display potent functional activation of all three receptors, namely the human GLP-1 receptor, human GIP receptor, and human amylin receptor (hAMYR3).

Most of the GLP-1-/GIP-/amylin-receptor tri-agonists of the present invention agonize the different receptors with EC₅₀ values comparable to the GIP, GLP-1 and amylin receptor agonists and GLP-1/GIP co-agonists disclosed herein as reference compounds 4 to 7. Furthermore, the GLP-1-/GIP-/amylin-receptor tri-agonists of the present invention, as shown in Table 8a/b, display a potent and balanced functional activation of all three receptors, and are, in contrast to the reference compounds 1, 2 and 3, balanced GLP-1-/GIP-/amylin-receptor tri-agonists.

TABLE 9a
In vitro activity data for reference compounds
on hGLP-1R, hGIPR, and hAMYR3 measured (according
to methods A) in the presence of 1% HSA

| | hGLP-1R | hGIPR | hAMYR3 |
|---|---|---|---|
| Reference | Potency, | Potency, | Potency, |
| compound | 1% HSA, | 1% HSA, | 1% HSA, |
| number | EC₅₀ [pM] | EC₅₀ [pM] | EC₅₀ [pM] |
| 4 | nd | 78.27 | nd |
| 6 | nd | nd | 51.8 |
| 7 | 138.18 | nd | nd |

TABLE 9b
In vitro activity data for reference compounds
on hGLP-1R, hGIPR, and hAMYR3 measured (according
to methods B) in the presence of 1% HSA

| | hGLP-1R | hGIPR | hAMYR3 |
|---|---|---|---|
| Reference | Potency, | Potency, | Potency, |
| compound | 1% HSA, | 1% HSA, | 1% HSA, |
| number | EC₅₀ [pM] | EC₅₀ [pM] | EC₅₀ [pM] |
| 4 | nd | 80.21 | nd |
| 6 | nd | nd | 84.41 |
| 7 | 138.82 | nd | nd |

TABLE 10a
In vitro activity data for the synthesized compounds
of the invention on hGLP-1R, hGIPR, and hAMYR3 measured
(according to methods A) in the presence of 1% HSA

| | hGLP-1R | hGIPR | hAMYR3 |
|---|---|---|---|
| | Potency, | Potency, | Potency, |
| Compound | 1% HSA, | 1% HSA, | 1% HSA, |
| number | EC₅₀ [pM] | EC₅₀ [pM] | EC₅₀ [pM] |
| 10 | 245.54 | 42.37 | 174.93 |
| 11 | 204.33 | 58.3 | 211.21 |
| 12 | 10.22 | 59.11 | 55.04 |
| 14 | 75.92 | 254.17 | 33.38 |
| 15 | 102.87 | 356.21 | 82.21 |
| 16 | 238.41 | 485.28 | 165.52 |
| 17 | 13.14 | 36.93 | 33.58 |
| 26 | 1675.65 | 557.59 | 349.42 |
| 36 | 207.7 | 226.46 | 136.95 |
| 37 | 188.46 | 132.8 | 75.7 |
| 38 | 2347.55 | 335.89 | 171.67 |
| 39 | 1925.86 | 352.39 | 140.43 |
| 43 | 41.47 | 186.48 | 56.46 |
| 45 | 111.93 | 131.5 | 62.4 |
| 47 | 67.45 | 162.38 | 55.79 |
| 48 | 105.34 | 54.01 | 78.51 |
| 49 | 110.45 | 16.72 | 106.32 |
| 50 | 66.29 | 15.09 | 185.14 |
| 51 | 32.25 | 61.85 | 57.15 |
| 52 | 26.88 | 6.79 | 118.58 |
| 53 | 79.17 | 14.55 | 75.87 |
| 55 | 45.28 | 10.29 | 34.01 |
| 56 | 58.42 | 8.48 | 44.89 |
| 57 | 29.55 | 5.85 | 84.99 |
| 58 | 25.6 | 7.58 | 134.14 |
| 59 | 39.75 | 27.18 | 24.17 |
| 60 | 172.53 | 28.33 | 191.08 |
| 61 | 14.69 | 15.96 | 30.75 |
| 62 | 11.51 | 47.16 | 28.78 |
| 63 | 100.94 | 63.21 | 70.25 |
| 64 | 67.85 | 100.01 | 108.96 |
| 65 | 100.45 | 118.01 | 127.16 |
| 67 | 105.24 | 37.02 | 190.49 |
| 68 | 51.87 | 11.91 | 229.55 |
| 69 | 20.35 | 8.05 | 78.88 |
| 70 | 18.41 | 19.14 | 59.36 |
| 71 | 18.43 | 9.28 | 274.0 |
| 72 | 41.74 | 9.79 | 589.32 |
| 73 | 54.08 | 96.54 | 61.51 |
| 74 | 140.97 | 18.71 | 134.37 |
| 75 | 25.19 | 11.87 | 109.5 |
| 76 | 63.59 | 11.1 | 429.53 |
| 77 | 51.35 | 13.36 | 381.81 |
| 78 | 17.79 | 10.4 | 17.14 |
| 79 | 168.07 | 24.71 | 53.44 |
| 80 | 111.24 | 20.25 | 62.37 |
| 81 | 51.37 | 15.81 | 187.55 |
| 82 | 24.2 | 19.05 | 37.97 |
| 83 | 9.89 | 13.6 | 45.74 |
| 84 | 25.9 | 11.64 | 175.37 |
| 85 | 30.32 | 14.64 | 249.74 |
| 86 | 129.61 | 122.39 | 58.35 |
| 87 | 39.32 | 38.57 | 36.55 |
| 88 | 48.76 | 40.38 | 101.5 |
| 89 | 29.46 | 30.61 | 39.66 |
| 90 | 38.6 | 32.54 | 182.87 |
| 91 | 17.44 | 9.31 | 28.77 |
| 92 | 13.37 | 8.53 | 75.11 |
| 93 | 16.44 | 8.58 | 66.39 |
| 94 | 130.12 | 29.54 | 377.38 |
| 95 | 146.48 | 22.83 | 233.45 |
| 96 | 162.02 | 29.95 | 594.92 |
| 97 | 108.91 | 8.38 | 131.5 |
| 98 | 43.14 | 11.18 | 107.95 |
| 99 | 36.16 | 7.41 | 128.85 |
| 100 | 306.66 | 217.28 | 210.38 |
| 101 | 179.06 | 128.91 | 95.53 |
| 102 | 46.99 | 12.19 | 134.0 |
| 103 | 33.14 | 13.31 | 73.6 |
| 104 | 19.09 | 17.29 | 293.01 |
| 105 | 48.9 | 12.5 | 113.18 |
| 106 | 11.07 | 10.27 | 31.24 |
| 107 | 13.92 | 8.76 | 93.1 |
| 108 | 15.0 | 11.45 | 59.95 |
| 109 | 94.68 | 17.44 | 31.96 |
| 110 | 169.38 | 29.31 | 93.98 |
| 111 | 34.79 | 12.22 | 66.36 |
| 112 | 335.79 | 321.43 | 268.55 |
| 113 | 57.52 | 8.96 | 136.32 |
| 114 | 126.11 | 17.18 | 45.2 |

TABLE 10b
In vitro activity data for the synthesized compounds
of the invention on hGLP-1R, hGIPR, and hAMYR3 measured
(according to methods B) in the presence of 1% HSA

| | hGLP-1R | hGIPR | hAMYR3 |
|---|---|---|---|
| | Potency, | Potency, | Potency, |
| Compound | 1% HSA, | 1% HSA, | 1% HSA, |
| number | EC₅₀ [pM] | EC₅₀ [pM] | EC₅₀ [pM] |
| 115 | 92.9 | 38.92 | 551.2 |
| 120 | 128.7 | 20.0 | 774.1 |
| 121 | 47.41 | 15.98 | 472.3 |
| 122 | 1120 | 116.0 | 147.0 |
| 123 | 1190 | 89.5 | 90.3 |
| 124 | 1120 | 152.0 | 228.5 |
| 125 | 89.3 | 12.62 | 555.0 |
| 126 | 865.0 | 102.0 | 129.7 |
| 127 | 276.7 | 98.45 | 88.0 |
| 128 | 165.0 | 17.0 | 429.0 |
| 129 | 998.0 | 72.6 | 62.6 |
| 130 | 1140 | 99.1 | 220.0 |
| 131 | 225.0 | 27.2 | 949.0 |
| 132 | 64.9 | 12.7 | 568.0 |
| 133 | 105.0 | 12.7 | 917.0 |
| 134 | 220.0 | 34.7 | 1200 |
| 135 | 123.0 | 18.6 | 568.0 |
| 136 | 120.8 | 20.53 | 629.0 |
| 137 | 123.0 | 13.0 | 704.0 |
| 138 | 56.2 | 8.0 | 555.0 |
| 139 | 59.7 | 6.0 | 489.0 |
| 140 | 200.0 | 19.6 | 878.0 |
| 141 | 119.1 | 11.69 | 771.3 |
| 142 | 102.0 | 9.99 | 453.0 |
| 143 | 93.5 | 12.7 | 483.0 |
| 144 | 76.07 | 8.05 | 778.8 |
| 145 | 111.0 | 15.2 | 6130 |
| 146 | 92.5 | 12.9 | 842.0 |
| 147 | 83.0 | 11.0 | 837.3 |
| 148 | 72.71 | 7.979 | 727.8 |
| 149 | 88.4 | 16.3 | 1060 |
| 150 | 65.3 | 10.7 | 961.0 |
| 151 | 72.6 | 13.9 | 949.5 |
| 152 | 67.4 | 11.4 | 913.8 |
| 153 | 66.49 | 10.48 | 912.9 |
| 154 | 80.6 | 14.2 | 1145 |
| 155 | 55.6 | 14.0 | 652.0 |
| 156 | 91.8 | 26.5 | 876.0 |
| 157 | 77.4 | 16.3 | 636.0 |
| 158 | 112.0 | 19.6 | 638.0 |
| 159 | 103.0 | 14.1 | 716.0 |
| 160 | 55.9 | 8.7 | 715.0 |
| 161 | 54.2 | 7.7 | 1150 |
| 162 | 61.2 | 6.00 | 988.0 |
| 163 | 68.12 | 7.43 | 2230 |
| 164 | 60.27 | 7.59 | 1180 |
| 165 | 88.79 | 7.64 | 537.0 |
| 166 | 54.78 | 6.19 | 1296 |
| 167 | 66.49 | 5.85 | 1341 |
| 168 | 63.0 | 6.42 | 914.9 |
| 169 | 517.0 | 74.1 | 184.0 |
| 170 | 685.0 | 36.8 | 127.0 |
| 171 | 468.0 | 39.0 | 110.0 |
| 172 | 578.0 | 85.2 | 353.0 |
| 173 | 750.0 | 63.8 | 290.0 |
| 174 | 602.0 | 93.2 | 1650 |
| 175 | 731.0 | 82.3 | 407.0 |
| 176 | 178.0 | 23.8 | 798.0 |
| 177 | 181.0 | 18.7 | 779.0 |
| 178 | 173.0 | 17.0 | 1480 |
| 179 | 170.0 | 25.5 | 1350 |
| 180 | 80.49 | 11.9 | 1103 |
| 18 | 134.5 | 20.1 | 1300 |
| 182 | 139.4 | 18.64 | 2743 |
| 183 | 744.4 | 71.41 | 108.1 |
| 184 | 718.1 | 61.57 | 122.6 |
| 185 | 954.2 | 72.34 | 192.0 |
| 186 | 829.7 | 85.46 | 208.0 |
| 187 | 1110 | 123.0 | 476.0 |
| 188 | 1154 | 153.8 | 1036 |
| 189 | 81.9 | 9.18 | 1210 |
| 190 | 21.8 | 3.9 | 386.0 |
| 191 | 26.0 | 6.4 | 815.0 |
| 192 | 55.7 | 8.22 | 291.0 |
| 193 | 110.0 | 12.0 | 275.0 |
| 194 | 763.0 | 15.7 | 1660 |
| 195 | 528.0 | 9.52 | 1690 |
| 196 | 201.0 | 21.6 | 641.0 |
| 197 | 148.0 | 18.4 | 882.0 |

### Example 5: Experimental Protocol for Efficacy Testing on Appetite Using an Ad Libitum Fed Rat Model

Male Sprague Dawley (SD) rats from Taconic, Denmark were used for the acute food intake experiments, wherein the principles of laboratory animal care were followed.

The rats were of normal weight of 250-350 g at start of experiment. The rats arrived at least 10-14 days before the start of the experiment to allow acclimatization to experimental settings. During this period, the animals were handled (fixation by restrain in neck skin) at least 2 times. Immediately upon arrival, rats were changed to a reversed light cycle (dark from 11 am-11 pm) and were transferred to an automated food intake measuring system (HM2 system, MBRose; Faaborg, Denmark). Rats had ad libitum access to chow (Altromin cat. No. 1324, Brogaarden, Lynge, Denmark) and water, and were housed at room temperature (˜22° C.). To enable recording of individual food intake, rats were ID chipped. Three rats were housed in each cage. During the acclimatization period, in which the rats get used to the new light cycle and diet (LF 10% (D12450B), from Research Diets Inc.), the animals had free access to food and water. Since rats are normally active and consume most of their daily calories during the dark period, rats were dosed in the morning right before lights were turned off. Such a set-up results in the lowest data variation and highest test sensitivity. Each dose of tri-agonist was tested in a group of 5-8 rats. A vehicle group of 6-8 rats was included in each set of testing. In each cage there were animals from three different treatment groups (to eliminate potential cage effects (e.g. cage malfunction) on primary readout: food intake). The rats were dosed once subcutaneously (s.c.) with the peptide of interest according to body weight (10 or 30 nmol/kg) in vehicle (0.5 ml/kg) using a NovoPen® (Novo Nordisk, Bagsvæed, Denmark).

The compounds of the invention were formulated (20 or 60 nmol/ml) in the following vehicle: 8 mM phosphate; 250 mM glycerol; 0.007% polysorbate 20, pH 7.4.

After dosing, the rats were returned to their home cages, where they continued to have ad libitum access to chow and water. The food consumption was recorded individually and continuously by the HM2 system from 0-72 h after test compound administration. Data obtained by the HM-2 system were stored in a HMBase SQL database (Firebird® relational database management system) and were processed by HM2Lab software (MBRose; Faaborg, Denmark) installed in an embedded computer. The feeding system is a highly sensitive system with a load resolution of 0.001 g. In addition to recording food intake, the system records number of feeding events, which is defined as a 0.001 g reduction of food within 5s (Detailed information's of the system can be found in Rathod, Y. D., and Di Fulvio, M. (2021). The feeding microstructure of male and female mice. PLoS One 16, e0246569.) At the end of the experimental session, the animals were euthanized.

Table 11 shows acute food intake in normal weight (lean) rats based on above-described protocol for efficacy testing on appetite. The results allowed assessment of in vivo effect on food intake and provided an indication of the compounds' duration of action. Data are expressed as average percent inhibition relative to average food intake in vehicle group at each study day (day 1 [0-24 hours], day 2 [24-48 hours] and day 3 [48-72 hours]) and food intake was performed in rats up to 72 h. Food intake at each study day (e.g. day 1) means cumulative food intake during, i.e. over the course of this day (i.e. the 24 hours period).

TABLE 11
Results for acute food intake in normal weight
rats after a single dose of tri-agonist

| | Food intake (relative to vehicle in [%]) | | | | | |
|---|---|---|---|---|---|---|
| Compound | Day 1 | Day 2 | Day 3 | Day 1 | Day 2 | Day 3 |
| number | 10 nmol/kg | | | 30 nmol/kg | | |
| 10 | −20* | −4 | −4 | | | |
| 11 | | | | −46 | −45 | −27 |
| 12 | −70 | −39 | −8 | | | |
| 14 | −78 | −85 | −32 | | | |
| 15 | −30 | −42 | −9 | | | |
| 16 | −17 | −11 | −10 | −39 | −22 | −17 |
| 17 | −84 | −78 | −11 | | | |
| 26 | | | | −11 | −18 | 0 |
| 35 | | | | −42 | −18 | −16 |
| 36 | −44 | −45 | −3 | | | |
| 37 | −66 | −80 | −24 | | | |
| 38 | −3 | −19 | 1 | | | |
| 50 | −54 | −60 | −21 | | | |
| 52 | −80 | −93 | −35 | | | |
| 53 | −75 | −90 | −33 | | | |
| 55 | −85 | −78 | −27 | | | |
| 56 | −69 | −77 | −10 | | | |
| 57 | −75 | −88 | −14 | | | |
| 58 | −71 | −90 | −18 | | | |
| 61 | −86 | −92 | −34 | | | |
| 62 | −74 | −42 | −13 | | | |
| 63 | −79 | −80 | −17 | | | |
| 68 | −65 | −72 | −35 | | | |
| 69 | −81 | −68 | −6 | | | |
| 77 | −54 | −89 | −54 | | | |
| 78 | −96 | −57 | −3 | | | |
| 79 | −70 | −54 | −5 | | | |
| 80 | −84 | −78 | −23 | | | |
| 82 | −94 | −73 | −27 | | | |
| 87 | −91 | −86 | −24 | | | |
| 89 | −93 | −78 | −28 | | | |
| 91 | −96 | −84 | −33 | | | |
| 93 | −94 | −61 | −25 | | | |
| 97 | −65 | −82 | −42 | | | |
| 99 | −69 | −84 | −46 | | | |
| 100 | −42 | −64 | −40 | | | |
| 101 | −76 | −92 | −63 | | | |
| 109 | −85 | −34 | −28 | | | |
| 110 | −75 | −54 | −23 | | | |
| 115 | −44.5 | −76.5 | −38 | | | |
| 120 | −42 | −57 | −13 | | | |
| 121 | −51 | −85 | −52 | | | |
| 122 | −44 | −44 | −11 | | | |
| 124 | −24 | −35 | 4 | | | |
| 125 | −53 | −58 | −10 | | | |
| 126 | −23 | −37 | 6 | | | |
| 129 | −68 | −67 | −32 | | | |
| 130 | −61 | −58 | 1 | | | |
| 131 | −27 | −56 | −8 | | | |
| 132 | −36 | −67 | −16 | | | |
| 133 | −27 | −35 | −8 | | | |
| 134 | −10 | −25 | 0 | | | |
| 136 | −12 | −19 | 27 | | | |
| 137 | −24 | −49 | 4 | | | |
| 144 | −38 | −6 | −11 | | | |
| 145 | −31 | −52 | −14 | | | |
| 146 | −55 | −66 | −23 | | | |
| 147 | −32 | −62 | −2 | | | |
| 148 | −34 | −38 | −5 | | | |
| 152 | −26 | −30 | 10 | | | |
| 176 | −12 | 2 | 7 | | | |

*“−20” means that food intake is reduced by 20% when compared to vehicle. In general, “−X” means that food intake is reduced by “X %” when compared to vehicle.

Following dosing of the GLP-1-/GIP-/amylin-receptor tri-agonists of the present invention to rats, it was observed that many of them induced profound food intake inhibition, compared to vehicle treatment, as can be deduced from the data presented in Table 11. In general, the compounds in Table 11 showed similar or improved reduction in food intake compared to reference compound 1 as disclosed in WO 2023/288313 at a much lower dose than the disclosed dosage level of 50 nmol/kg of reference compound 1.

### Example 6: Pharmacokinetic Study in Minipigs

The purpose of this study is to determine the half-life (t_{1/2}) in vivo of the inventive GLP-1-/GIP-/amylin-receptor tri-agonists after intravenous (i.v.) administration to minipigs, i.e., the residence time in the body and thereby their time of action. This is done in a pharmacokinetic (PK) study, where the terminal half-life (t_{1/2}) of the compound or derivative in question is determined. By terminal half-life is meant the time it takes to halve a certain plasma concentration in the terminal elimination phase.

### Study

Female Göttingen minipigs, obtained from Ellegaard Göttingen Minipigs (Dalmose, Denmark), approximately 8-12 months of age and weighing approximately 20-30 kg were used in the studies. The minipigs were housed individually (pigs with permanent catheters) in pens with straw as bedding and fed restrictedly once daily with Altromin 9023 minipig diet (Altromin Spezialfutter GmbH & Co. KG).

After three weeks of acclimatisation two permanent central venous catheters were implanted in vena cava caudalis in each animal. The animals were allowed at least 10 days recovery after the surgery and were then used for repeated pharmacokinetic studies with a suitable wash-out period between successive dosing.

The compounds of the invention were formulated (40 nmol/ml) in the following vehicle: 8 mM phosphate; 250 mM glycerol; 0.007% polysorbate 20, pH 7.4.

Intravenous injections (the volume corresponding to 0.05 ml/kg and dose of 2 nmol/kg/derivative) of the derivatives were given through one catheter, and blood was sampled at predefined time points for up till 14 days post dosing (preferably from the other catheter).

Blood samples (for example 1.3 ml) were collected in EDTA (1.3 ml tube containing K₃EDTA to yield 1.6 mg K₃EDTA/ml blood) coated tubes and then centrifuged at 4° C. and 2000×g for 10 minutes.

### Sampling and Analysis

Plasma was within 30 min after centrifugation pipetted into Micronic tubes stored on dry ice and were afterwards kept at −20° C. until analysed for plasma concentration of the compounds using LCMS.

Plasma concentrations of the peptides of the invention were assayed by plasma protein precipitation and analysed by liquid chromatography mass spectrometry (LC-MS). Calibrators were prepared by spiking blank plasma from minipigs with the compounds in the typical range from 0.05 to 200 nM. LLoQ was typically in the range of 0.2-2 nM. Calibrators, plasma blanks or study samples were prepared for LC-MS by protein precipitation by adding 4 volumes of ethanol containing 20 nM of internal standard (structurally similar analogue with different mass) to one volume of sample followed by centrifugation at 6200 rpm at 4° C. for 10 minutes. The supernatant was diluted with 1 volumes of Milli-Q water containing 1% Formic acid before injection on the LC-MS system. Individual plasma concentration-time profiles were analysed by a non-compartmental pharmacokinetic method (NCA) in Phoenix v. 6.4 (Pharsight Inc., Mountain View, CA, USA), and the resulting terminal half-lives (harmonic mean) determined. The LC-MS analysis was carried out using a TurboFlow HPLC system from Thermo Fisher Scientific (Bremen, Germany) coupled to either a Q Exactive Orbitrap or Altis Triple Quadrupole (TQ) Mass Spectrometer. The LC mobile phases consisted of A: MQ water with 5% organic solvent (50% methanol/50% acetonitrile) and 1% formic acid and B: MQ water with 95% organic solvent (50% methanol/50% acetonitrile) and 1% formic acid. A TurboFlow Cyclone 0.5×100 mm column from Thermo Fischer Scientific (Bremen, Germany) was used for extraction, before analytical elution on a XBridge Peptide BEH C18 300 Å, 3.5 μm, 2.1×50 mm column for the analysis both operated at 60° C. Typically 40-45% B and 75-80% B were used for loading and elution on the TurboFlow column, respectively, followed by a linear gradient elution typically from around 45% B to 85% B over 2.33 minutes on the analytical column. The Orbitrap mass spectrometer were operating in positive ionization mode with a spray voltage of 4.0 kV using Parallel Reaction Monitoring scan mode using 5 m/z isolation windows on the most abundant charge state of the compounds with a resolution of 35K on the Orbitrap MS. The TQ mass spectrometer were operating in positive ionization mode with a spray voltage of 4.0 kV using Single Reaction Monitoring scan mode using Q1 and Q3 resolutions of 1.2 (FWHM).

For all compounds, individual optimal fragmentation collision energies were found and used. The data was processed using the Quan Browser in the Xcalibur software from Thermo Fisher Scientific (Bremen, Germany) by fitted the data to linear calibration curves (weighed 1/x²), used for calculating the concentration in the plasma samples. Quality control samples were included. The deviation between nominal and calculated concentration in the calibrators and quality control samples were below 15%.

### Results:

TABLE 12
Terminal half-life as measured after i.v. administration to minipigs
Terminal half-life as measured after i.v. administration to minipigs

| Compound number | t_{1/2} [hours] |
|---|---|
| 11 | 119 |
| 12 | 81 |
| 15 | 122 |
| 16 | 138 |
| 37 | 118 |
| 52 | 120 |
| 55 | 88 |
| 57 | 90 |
| 58 | 97 |
| 61 | 61 |
| 64 | 117 |
| 68 | 125 |
| 77 | 140 |
| 91 | 43 |
| 93 | 69 |
| 101 | 122 |
| 115 | 115 |
| 120 | 95 |
| 125 | 100 |

As shown in Table 12, the tested GLP-1-/GIP-/amylin-receptor tri-agonists of the present invention have very long half-lives (t_{1/2}) of at least 43 hours in minipigs. It is contemplated that, based on these half-lives in minipigs, half-lives in humans will be sufficient for at least once-weekly administration via liquid subcutaneous injection or at least once daily administration via oral tablet.

### Example 7: Chemical Stability Assessment in Formulation

The assay was performed to investigate the extend of chemical degradation in vitro over time upon incubation at 37° C. for a period of 2 weeks.

Peptide solutions were prepared by dissolving freeze-dried powder in 8 mM phosphate buffer pH 7.4 to a target of 1 mg/mL. The pH of peptide solutions was adjusted to 7.4 with 0.02 M HCl or 0.02 M NaOH. Samples were filled in Agilent HPLC vials with fixed insert. Vials were capped to prevent evaporation. The HPLC vials were incubated at 37° C. and samples were withdrawn at different time points over a period of 2 weeks, flash frozen at −80° C., and stored at −20° C. until analysis.

Sample analysis was carried out using UPLC coupled to UV detection at 215 nm and MS (UPLC-UV-MS). One μL of sample was injected into a Waters Acquity UPLC with a flow-through-needle injection system and on to a Waters Acquity CSH C18 column (1*150 mm), with a particle size of 1.7 μm and held at 55° C. A flow-rate of 100 μL/min was delivered with a Binary solvent manager pump having 0.1% formic acid in water as solvent A and 0.1% formic acid in acetonitrile as solvent B. Gradient elution was carried out using 20% B from 0 to 2 min followed by 20 to 50% B from 2 to 20 min in a total run time of 30 min. The identity of the peptide was confirmed by MS and the peak purity, area %, from the UV signal at 215 nm was plotted against time and the slope from linear regression was used to calculate the purity loss per week (Table 13).

TABLE 13
Chemical stability assessment of peptides in
8 mM phosphate buffer pH 7.4 (1 mg/mL) at 37°
C. Data is given as purity loss in % per week.

| Compound | Purity loss per week at |
|---|---|
| number | 37° C. [in %] |
| 12 | 5.6 |
| 15 | 3.8 |
| 17 | 4.2 |
| 35 | 0.3 |
| 37 | 2.3 |
| 47 | 2.6 |
| 50 | 0.7 |
| 52 | 0.9 |
| 53 | 0.8 |
| 55 | 0.9 |
| 56 | 0.6 |
| 57 | 0.2 |
| 58 | 0.3 |
| 61 | 2.0 |
| 62 | 3.0 |
| 63 | 3.7 |
| 64 | 3.3 |
| 65 | 2.1 |
| 73 | 2.8 |
| 77 | 1.2 |
| 78 | 3.5 |
| 79 | 1.3 |
| 80 | 0.8 |
| 83 | 2.3 |
| 84 | 2.9 |
| 85 | 2.0 |
| 91 | 2.8 |
| 92 | 2.8 |
| 93 | 2.8 |
| 115 | 1.1 |
| 120 | 1.2 |
| 121 | 1.5 |
| 122 | 1.3 |
| 123 | 1.0 |
| 124 | 1.5 |
| 125 | 0.5 |
| 126 | 1.2 |
| 127 | 1.7 |
| 129 | 1.7 |
| 130 | 1.6 |
| 132 | 1.3 |
| 133 | 1.4 |
| 134 | 1.4 |
| 146 | 1.4 |
| 147 | 1.0 |
| 148 | 1.4 |
| 149 | 1.1 |
| 150 | 1.6 |
| 151 | 1.3 |
| 152 | 1.3 |
| 153 | 1.4 |
| 154 | 1.4 |
| 160 | 1.2 |
| 161 | 0.9 |
| 162 | 1.0 |
| 163 | 1.4 |
| 164 | 1.2 |
| 165 | 1.0 |
| 171 | 1.0 |
| 172 | 0.9 |
| 180 | 0.7 |
| 183 | 1.3 |
| 184 | 1.0 |
| 185 | 1.7 |
| 186 | 1.0 |

All GLP-1-/GIP-/amylin-receptor tri-agonists tested in this assay show an acceptable chemical stability with an acceptable rate of degradation (less than 6% of purity loss per week) in an aqueous buffer (at 37° C.). Most of the tested GLP-1-/GIP-/amylin-receptor tri-agonists show a good chemical stability having less than 3.0% purity loss per week, or even more an excellent chemical stability (less than 1.5% purity loss per week). Accordingly, the GLP-1-/GIP-/amylin-receptor tri-agonists of the present invention are considered to be chemically stable in solution.

### Example 8: Sub-Chronic Treatment in Diet-Induced Obese (DIO) Rats

The purpose of this example is to assess the in vivo effect of selected tri-agonists on pharmacodynamic parameters such as body weight and food intake in diet-induced obese (DIO) rats. The animals were treated once daily via subcutaneous injection with a liquid formulation of the triple agonist to be tested to assess effects on body weight and food intake (daily and cumulative).

Diet-induced obese male rats (Sprague Dawley) were purchased from Charles River (Écully, France). Rats were housed in pairs of two at room temperature with ad libitum access to 45% high fat diet (D12451, Research Diets, Inc. NJ) initially following a 12 h:12h light:dark cycle with lights on at 0600h. Two weeks before start of study, rats were switched to reverse light:dark cycle (12:12h) with lights on at 21.00-09.00h. Five days prior to start of treatment, animals were weighted and MRI scanned (EchoMRI™, TX, USA) to obtain body composition data. Rats were divided into seven groups (n=8) matched on body weight and fat mass (P>0.91 between groups for both body weight and fat mass as tested by Oneway ANOVA followed by Tukey Multiple comparison test), ensuring that no cage had two rats from the same group. Group details are shown in Table 14 below. A group of age matched normal weight controls, who had been on standard chow (Altromin 1324, Altromin International) for the entirety of their lives was included as reference. At start of study, average weight of DIO rats was 958±16.4 g (mean±SEM). Average weight of normal weight controls was 796±21.4 g (mean±SEM). Body weight and daily food intake was collected by manual weighing (0-1 h before onset of dark during four days prior to start of treatment (pre-treatment/baseline phase). In continuation to this, rats were mock handed to accustom them to the dosing procedure during treatment (restrain in neck skin). During baseline and treatment phases, food was replaced daily. Treatment phase lasted four weeks (28 days) with daily subcutaneous injection (QD, s.c.) at a volume of 0.5 ml/kg.

The compounds of the invention were formulated (0.6 nmol/ml, 2 nmol/ml, 6 nmol/ml, 12 nmol/ml, and 20 nmol/ml, depending on administered dose) in the following vehicle: 8 mM phosphate; 250 mM glycerol; 0.007% polysorbate 20, pH 7.4. Rats were dosed with a NovoPen® (Novo Nordisk, Bagsværd, Denmark) 0-1 h before start of dark phase immediately after weighing. The two test compounds were titrated in small increments towards maintenance doses of either 3 nmol/kg or 10 nmol/kg as shown in Table 14.

TABLE 14
Dose titration schedule for DIO rat study described
in Table 15 (Dosing in nmol/kg body weight)

| Compound | n | Day 1 + 2 | Day 3 + 4 | Day 5 + 6 | Day 7 + 8 | Day 9 − 28 |
|---|---|---|---|---|---|---|
| Vehicle | 8 | 0 | 0 | 0 | 0 | 0 |
| Compound 52, 3 nmol/kg | 8 | 0.3 | 1 | 3 | 3 | 3 |
| Compound 52, 10 nmol/kg | 8 | 0.3 | 1 | 3 | 6 | 10 |
| Compound 77, 3 nmol/kg | 8 | 0.3 | 1 | 3 | 3 | 3 |
| Compound 77 10 nmol/kg | 8 | 0.3 | 1 | 3 | 6 | 10 |

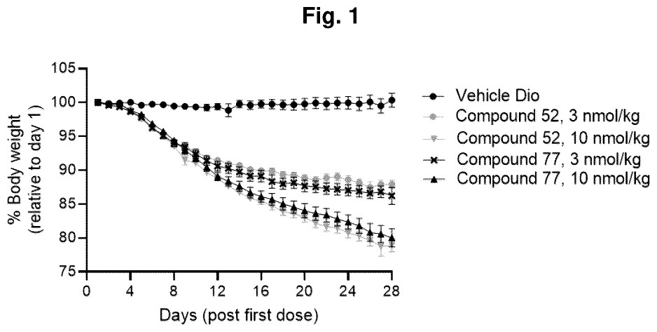

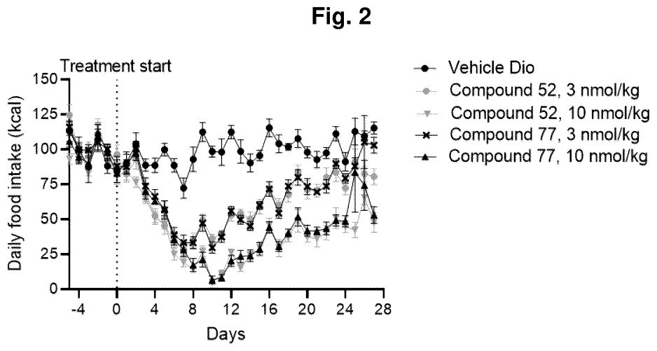

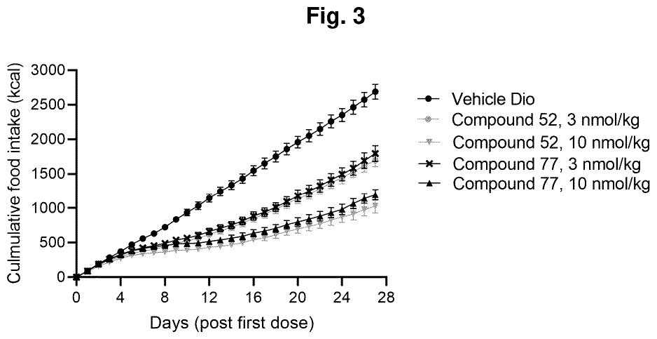

The results of this study are shown in FIG. 1, FIG. 2, and FIG. 3, and in Table 15. Table 15 shows the effects on cumulative food intake, absolute body weight and relative body weight of DIO rats treated daily for up to 28 days with vehicle and compound 52 or 77 of the present invention (at 3 nmol/kg and 10 nmol/kg, respectively) following the dose titration schedule shown in Table 14.

TABLE 15
Effects on food intake and body weight in DIO rats treated daily
for up to 28 days with vehicle, compound 52 or compound 77
following the dose titration schedule shown in Table 14

| | | | | body |
|---|---|---|---|---|
| | | | | weight |
| | | | | in [%] |
| | Cumulative | | | at day 28 |
| | food intake | Absolute body | | compared |
| | in [kcal] | weight in [g] | | to day 0 |
| Compound | Day 28 | Day 0 | Day 28 | Day 28 |
| Vehicle, | 2688 ± 109.4 | 968 ± 61.6 | 969 ± 56.9 | 100 ± 1.04 |
| DIO | | | | |
| 77 | 1795 ± 116.9 | 962 ± 45.5 | 831 ± 47.8 | 86.2 ± 1.27 |
| (3 nmol/kg) | | | | |
| 77 | 1193 ± 75.60 | 954 ± 37.1 | 765 ± 37.9 | 80.0 ± 1.35 |
| (10 nmol/kg) | | | | |
| 52 | 1696 ± 92.90 | 949 ± 32.7 | 836 ± 32.3 | 87.8 ± 0.61 |
| (3 nmol/kg) | | | | |
| 52 | 1028 ± 99.45 | 940 ± 32.6 | 742 ± 29.1 | 78.9 ± 0.97 |
| (10 nmol/kg) | | | | |

Results are expressed as means ± SEM, n = 6-8.

The second column in Table 15 shows cumulative food intake from treatment day 0-28 (in kcal). The DIO rats received a subcutaneous dose once daily following the titration schedule described in Table 14. Data are shown as means±SEM, n=6-8. These results are also shown in FIG. 3. It can be seen that treatment with the GLP-1-/GIP-/amylin-receptor triple agonist compounds 52 and 77 induced a reduction in food intake.

The third column in Table 15 shows absolute body weight (in g) of the rats, dosed with vehicle, compound 52 or compound 77 (at 3 nmol/kg and 10 nmol/kg, respectively) at day 0 and day 28 of the treatment period. The DIO rats received a subcutaneous dose once daily following the titration schedule described in Table 14. Data are shown as means±SEM, n=6-8. The last column in Table 15 shows body weight relative to day 0 in percent (% body weight) in DIO rats during the treatment period of 28 days with compounds 52 and 77 (at 3 nmol/kg and 10 nmol/kg, respectively). For example, a relative body weight of 80% at day 28 means that the rat possesses only 80% of its body weight at day 0, or in other words, the rat has lost 20% of its initial body weight (at day 0). The DIO rats received a subcutaneous dose once daily following the titration schedule described in Table 14. Data are shown as means±SEM, n=6-8. These results are also shown in FIG. 1. It can be seen that treatment with the GLP-1-/GIP-/amylin-receptor triple agonist compounds 52 and 77 induced a reduction in body weight in absolute and relative manner.

From Table 15 it is seen that treatment with the GLP-1-/GIP-/amylin-receptor triple agonist compounds 52 and 77 induced at all concentrations a reduction in food intake that resulted in body weight loss. The treatment with the GLP-1-/GIP-/amylin-receptor triple agonist compound 52 and compound 77 with same concentration (3 nmol/kg or 10 nmol/kg) induced a comparable reduction in food intake that resulted in a comparable body weight loss. The effects of reduction in food intake and body weight loss were concentration dependent, with a lower reduction in food intake and body weight loss at 3 nmol/kg and a higher reduction in food intake and body weight loss at 10 nmol/kg.

This invention is not limited to the particular methodologies, protocols and reagents described herein as these may vary without departing from the scope of the present disclosure. It is also to be understood that the terminology used herein is for the purpose of describing particular embodiments only, and is not intended to limit the scope of the present invention which will be limited only by the appended claims. Unless defined otherwise, all technical and scientific terms used herein have the same meanings as commonly understood by one of ordinary skill in the art.

In the above, the elements of the present invention were described. These elements were listed with specific embodiments. However, it should be understood that said embodiments may be combined in any manner and in any number to create additional embodiments, which all fall within the scope of the present disclosure. The various described examples and preferred embodiments should not be construed to limit the present invention to only the explicitly described embodiments. This description should be understood to support and encompass embodiments which combine the explicitly described embodiments with any number of the disclosed and/or preferred elements. Furthermore, any permutations and combinations of all described elements in this application should be considered disclosed by the description of the present application unless the context indicates otherwise.

Documents cited throughout the text of this specification (including all patents, patent applications, scientific publications, manufacturer's specifications, instructions, etc.), whether supra or infra, are hereby incorporated by reference in their entirety. Nothing herein is to be construed as an admission that the invention is not entitled to antedate such disclosure by virtue of prior invention.

While certain features of the invention have been illustrated and described herein, many modifications, substitutions, changes, and equivalents will now occur to those of ordinary skill in the art. It is, therefore, to be understood that the appended claims are intended to cover all such modifications and changes as fall within the true spirit of the invention.

1. A GLP-1-/GIP-/amylin-receptor tri-agonist comprising Z1-L1-Z2;
wherein
Z1 comprises
(SEQ ID NO: 161)
X₂₁X₂₂X₂₃GTFTSDYSX₂₄LLEEX₂₅AAX₂₆EFIX₂₇WLX₂₈X₂₉GGPS
X₃₀X₃₁;
wherein
X₂₁ represents Tyr (Y),
X₂₂ represents Aib,
X₂₃ represents Glu (E),
X₂₄ represents Ile (I),
X₂₅ represents Gln (Q),
X₂₆ represents Arg (R),
X₂₇ represents Glu (E),
X₂₈ represents Leu (L),
X₂₉ represents Ala (A),
X₃₀ represents Arg (R) or Ser (S),
X₃₁ represents Gly (G);
wherein
L1 is a peptide linker;
wherein
Z2 comprises
(SEQ ID NO: 164)
AX₃₂X₃₃LSTAX₃₄X₃₅X₃₆RLSAX₃₇LHX₃₈LX₃₉X₄₀X₄₁PX₄₂TETG
SGX₄₃P;
wherein
X₃₂ represents Ser (S),
X₃₃ represents His (H),
X₃₄ represents Gln (Q),
X₃₅ represents Thr (T),
X₃₆ represents Gln (Q),
X₃₇ represents Glu (E),
X₃₈ represents Lys (K),
X₃₉ represents Ala (A),
X₄₀ represents Thr (T),
X₄₁ represents Leu (L),
X₄₂ represents Arg (R),
X₄₃ represents Ser (S).

2. The GLP-1-/GIP-/amylin-receptor tri-agonist according to claim 1, wherein L1 comprises
X₁X₂X₃X₄X₅X₆X₇X₈X₉X₁₀X₁₁X₁₂X₁₃X₁₄,
wherein
X₁ represents Ala (A), Glu (E), or Gly (G),
X₂ represents Gln (Q), Glu (E), Gly (G), Leu (L), Pro (P), or is absent,
X₃ represents Ala (A), Gln (Q), Glu (E), Gly (G), Pro (P), or is absent,
X₄ represents Ala (A), Gln (Q), Glu (E), Gly (G), Pro (P), or is absent,
X₅ represents Glu (E), Gly (G), Pro (P), Ser (S), Thr (T), or is absent,
X₆ represents Glu (E), Gly (G), Leu (L), Gln (Q), or is absent,
X₇ represents Ala (A), Gln (Q), Glu (E), Gly (G), Phe (F), or is absent,
X₈ represents Ala (A), Gln (Q), Glu (E), Gly (G), Thr (T), Pro (P), Val (V), or is absent,
X₉ represents Glu (E), Asn (N), Pro (P), Thr (T), or is absent,
X₁₀ represents Ala (A), Gln (Q), Glu (E), Gly (G), Leu (L), Pro (P), Ser (S), Val (V), or is absent,
X₁₁ represents Ala (A) or is absent,
X₁₂ represents Gln (Q) or is absent,
X₁₃ represents Thr (T) or is absent,
X₁₄ represents Leu (L) or is absent.

3. The GLP-1/GIP/amylin-receptor tri-agonist according to claim 1, further comprising a protraction moiety attached to X₃₈ in Z2.

4. The GLP-1/GIP/amylin-receptor tri-agonist according to claim 3, wherein the protraction moiety is selected from the group consisting of

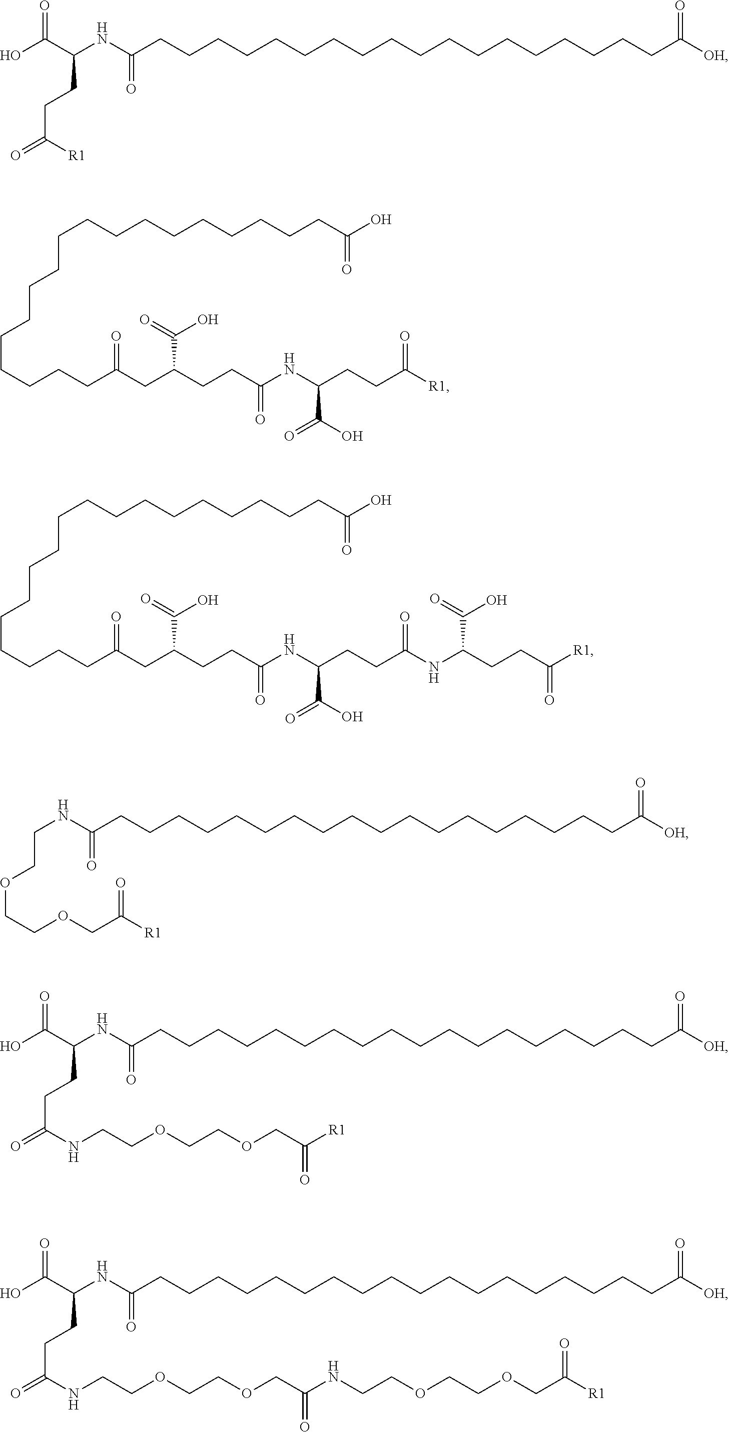

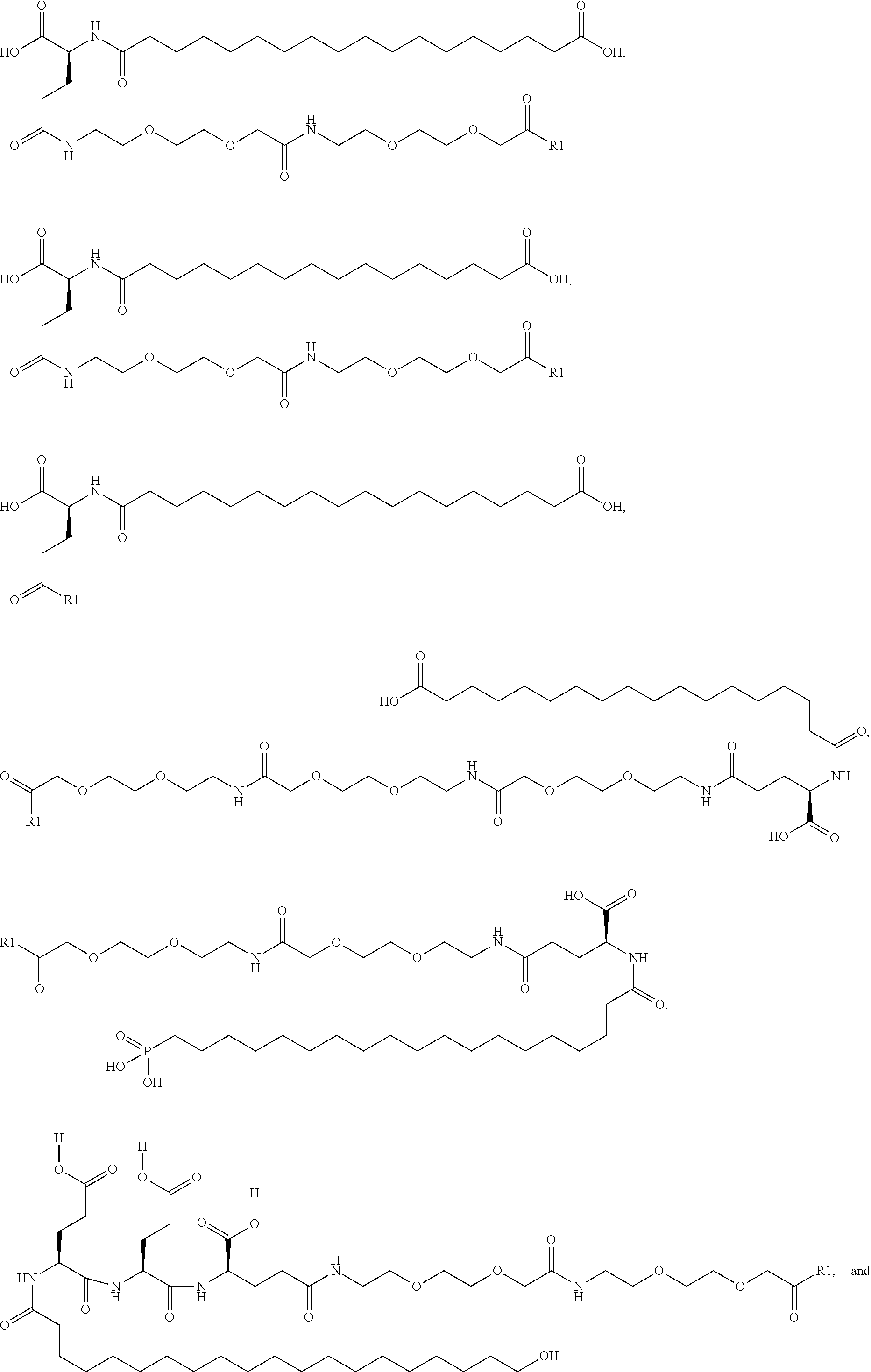

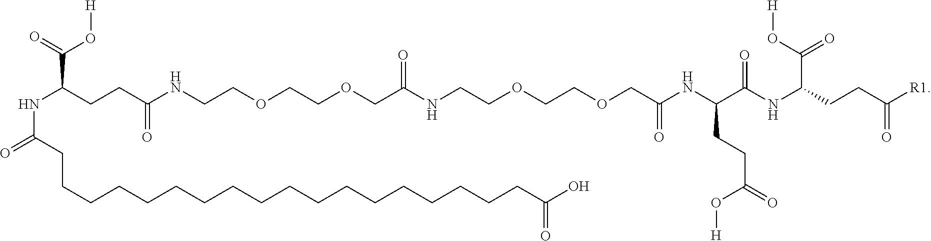

5. The GLP-1/GIP/amylin-receptor tri-agonist according to claim 4, wherein the protraction moiety is

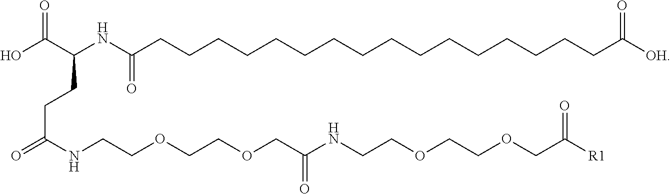

6. The GLP-1-/GIP-/amylin-receptor tri-agonist according to claim 1, wherein the amino acid sequence of the tri-agonist comprises
(SEQ ID NO: 62)
YXEGTFTSDYSILLEEQAAREFIEWLLAGGPSSGAGQAPGQAPGASHLST
AQTQRLSAELHKLATLPRTETGSGSP,
wherein X is Aib.

7. The GLP-1-/GIP-/amylin-receptor tri-agonist according to claim 1, wherein the amino acid sequence of the tri-agonist comprises
(SEQ ID NO: 68)
YXEGTFTSDYSILLEEQAAREFIEWLLAGGPSSGAGQAPGQAPLASHLST
AQTQRLSAELHKLATLPRTETGSGSP
wherein X is Aib.

8. The GLP-1-/GIP-/amylin-receptor tri-agonist according to claim 1, wherein the amino acid sequence of the tri-agonist comprises
(SEQ ID NO: 78)
YXEGTFTSDYSILLEEQAAREFIEWLLAGGPSRGEASHLSTAQTQRLSAE
LHKLATLPRTETGSGSP
wherein X is Aib.

9. The GLP-1-/GIP-/amylin-receptor tri-agonist according to claim 1, wherein the amino acid sequence of the tri-agonist comprises
(SEQ ID NO: 87)
YXEGTFTSDYSILLEEQAAREFIEWLLAGGPSSGAGQAPGQAPLASHLST
AQTQRLSAELHKLATLPRTETGSGSP
wherein X is Aib.

10. The GLP-1-/GIP-/amylin-receptor tri-agonist according to claim 1, wherein the tri-agonist is

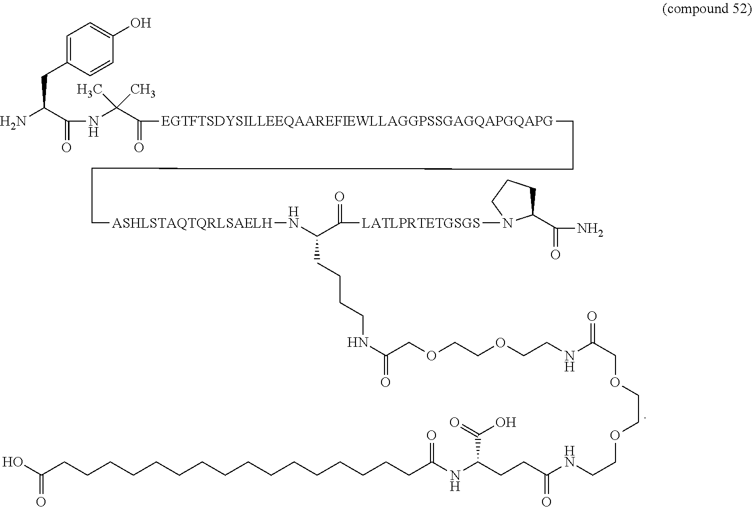

11. The GLP-1/GIP/amylin-receptor tri-agonist according to claim 1, wherein the tri-agonist is

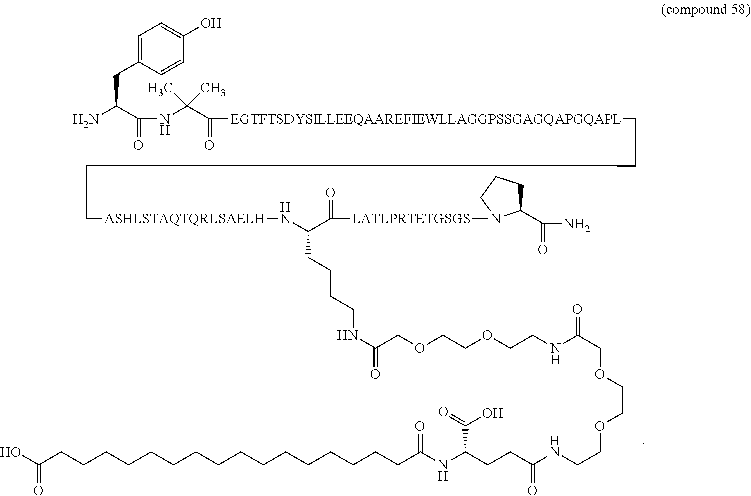

12. The GLP-1/GIP/amylin-receptor tri-agonist according to claim 1, wherein the tri-agonist is

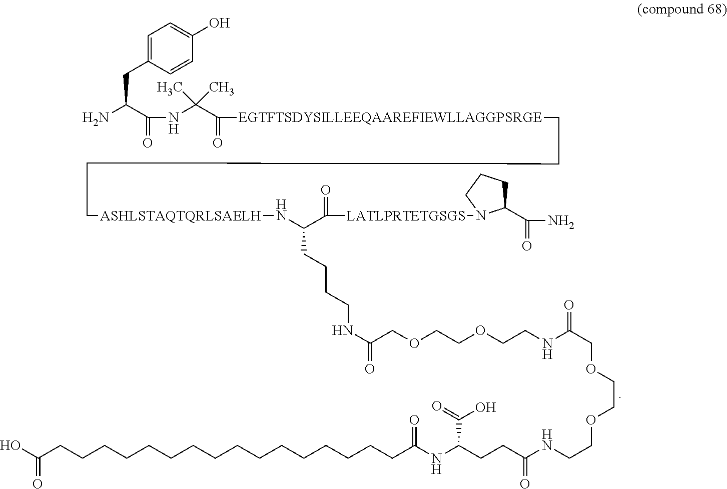

13. The GLP-1/GIP/amylin-receptor tri-agonist according to claim 1, wherein the tri-agonist is

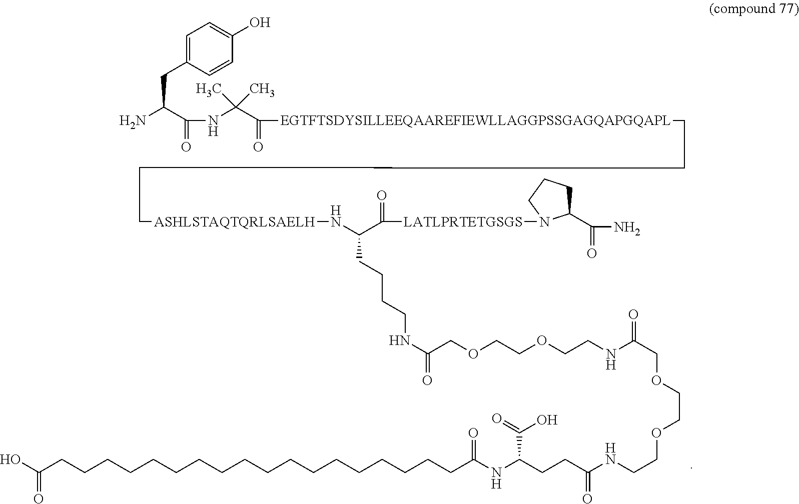

14. A method for reducing body weight in a subject in need thereof, comprising administering to the subject the GLP-1-/GIP-/amylin-receptor tri-agonist according to claim 1.

15. The method according to claim 14, wherein the subject is suffering from overweight.

16. The method according to claim 14, wherein the subject is suffering from obesity.

17. The method according to claim 14, wherein the tri-agonist is

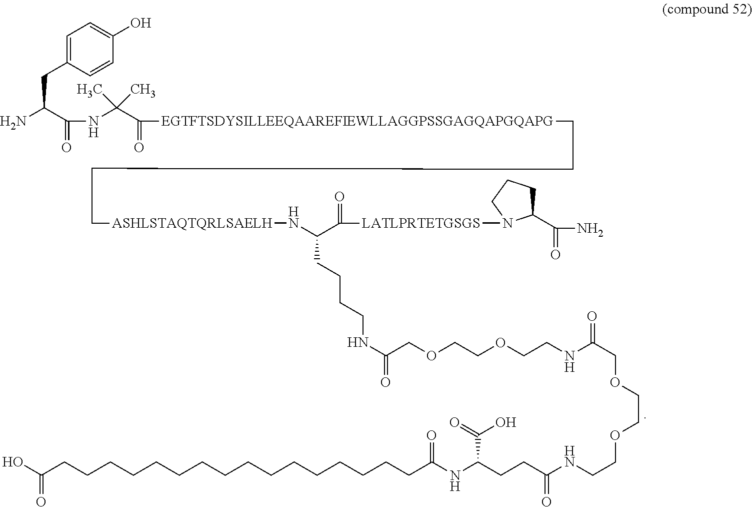

18. The method according to claim 17, wherein the subject is suffering from overweight.

19. The method according to claim 17, wherein the subject is suffering from obesity.

20. The method according to claim 14, wherein the tri-agonist is

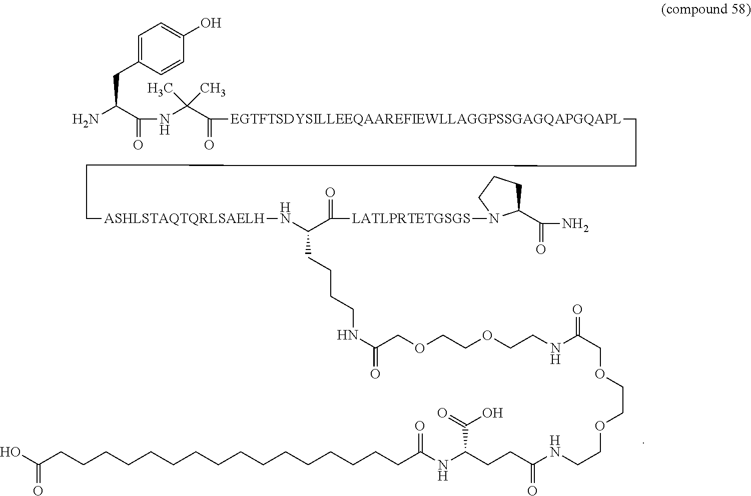

21. The method according to claim 20, wherein the subject is suffering from overweight.

22. The method according to claim 20, wherein the subject is suffering from obesity.

23. The method according to claim 14, wherein the tri-agonist is

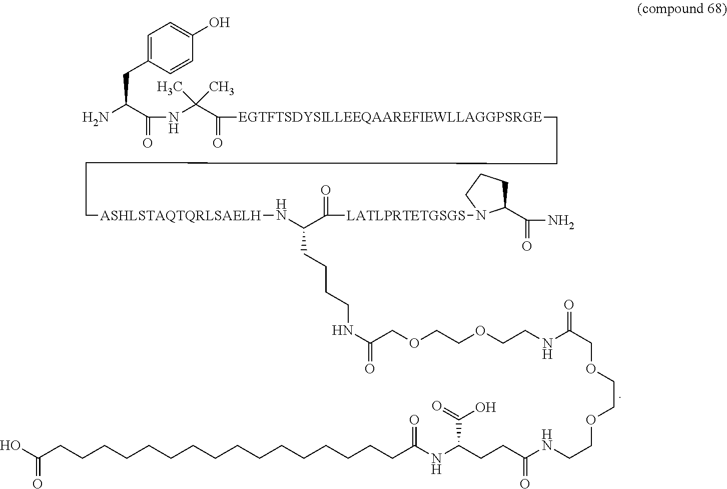

24. The method according to claim 23, wherein the subject is suffering from overweight.

25. The method according to claim 23, wherein the subject is suffering from obesity.

26. The method according to claim 14, wherein the tri-agonist is

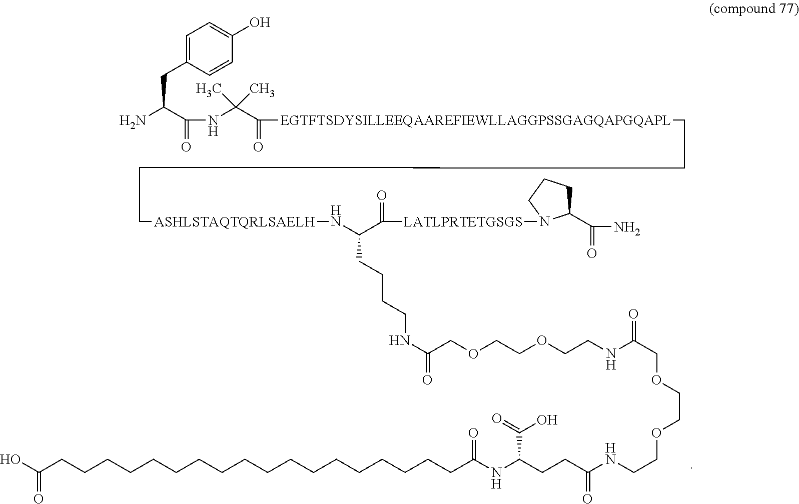

27. The method according to claim 26, wherein the subject is suffering from overweight.

28. The method according to claim 26, wherein the subject is suffering from obesity.


SEQUENCE LISTING (242 sequences)

SEQ ID NO: 1 - protein, 34 aa, synthetic construct
  VARIANT 2: note = Aib / 2-aminoisobutyric acid
YXEGTFTSDYSILLEEQAAREFIEWLLAGGPSKG

SEQ ID NO: 2 - protein, 32 aa, synthetic construct
ASELSTAALGRLSAELHELATLPRTETGSGSP

SEQ ID NO: 3 - protein, 34 aa, synthetic construct
  VARIANT 1: note = H, Y
  VARIANT 2: note = Aib/alpha-aminoisobutyric acid
  VARIANT 3: note = E, H
  VARIANT 33: note = R, G, K, S
  VARIANT 34: note = G, K
XXXGTFTSDYSILLEEQAAREFIEWLLAGGPSXX

SEQ ID NO: 4 - protein, 32 aa, synthetic construct
  VARIANT 3: note = Q, E, H, K,
  VARIANT 8: note = A, Q
  VARIANT 9: note = Q, L, T
  VARIANT 10: note = A, G, Q
  VARIANT 15: note = E, K
  VARIANT 18: note = R, Q, E, G, H, K, T, Y
  VARIANT 20: note = A, K
  VARIANT 21: note = D, T
  VARIANT 24: note = R, K
  VARIANT 31: note = A, S
ASXLSTAXXXRLSAXLHXLXXLPXTETGSGXP

SEQ ID NO: 5 - protein, 80 aa, synthetic construct
  VARIANT 1: note = H, Y
  VARIANT 2: note = Aib/alpha-aminoisobutyric acid
  VARIANT 3: note = E, H
  VARIANT 12: note = I, K
  VARIANT 17: note = Q, I
  VARIANT 20: note = R, Q
  VARIANT 24: note = A, E, Q
  VARIANT 27: note = L, I
  VARIANT 28: note = A, Q
  VARIANT 33: note = R, G, K, S
  VARIANT 34: note = G, E, K
  VARIANT 35: note = A, E, G
  VARIANT 36: note = Q, E, G, L, P or absent
  VARIANT 37: note = A, Q, E, G, P or absent
  VARIANT 38: note = A, Q, E, G, P or absent
  VARIANT 39: note = E, G, P, S, T or absent
  VARIANT 40: note = E, G, L, Q or absent
  VARIANT 41: note = A, Q, E, G, F or absent
  VARIANT 42: note = A, Q, E, G, T, P, V or absent
  VARIANT 43: note = E, N, P, T or absent
  VARIANT 44: note = A, Q, E, G, L, P, S, V or absent
  VARIANT 45: note = A or absent
  VARIANT 46: note = Q or absent
  VARIANT 47: note = T or absent
  VARIANT 48: note = L or absent
  VARIANT 50: note = G, S
  VARIANT 51: note = Q, E, H, K
  VARIANT 56: note = A, Q
  VARIANT 57: note = Q, L, T
  VARIANT 58: note = A, G, Q
  VARIANT 63: note = E, K
  VARIANT 66: note = R, Q, E, G, H, K, T, Y
  VARIANT 68: note = A, K
  VARIANT 69: note = D, T
  VARIANT 70: note = L, E
  VARIANT 72: note = R, K
  VARIANT 79: note = A, S
XXXGTFTSDYSXLLEEXAAXEFIXWLXXGGPSXXXXXXXXXXXXXXXXAXXLSTAXXXRLSAXLHXLXXXPXTETGSGXP

SEQ ID NO: 6 - protein, 34 aa, synthetic construct
  VARIANT 2: note = Aib/alpha-aminoisobutyric acid
  VARIANT 33: note = R, S
YXEGTFTSDYSILLEEQAAREFIEWLLAGGPSXG

SEQ ID NO: 7 - protein, 34 aa, synthetic construct
  VARIANT 2: note = Aib/alpha-aminoisobutyric acid
YXEGTFTSDYSILLEEQAAREFIEWLLAGGPSSG

SEQ ID NO: 8 - protein, 32 aa, synthetic construct
ASHLSTAQTQRLSAKLHRLATLPRTETGSGSP

SEQ ID NO: 9 - protein, 32 aa, synthetic construct
ASHLSTAQTQRLSAELHKLATLPRTETGSGSP

SEQ ID NO: 10 - skipped

SEQ ID NO: 11 - protein, 63 aa, synthetic construct
  source 1..63: note = D-tyrosine
  MOD_RES 63: note = C-terminal amide
  SITE 1: note = D-tyrosine
  DISULFID 33..38: note = Intrachain Disulfide bond
YAEGTFTSDYSIALDKIAQQAFVQWLLAGGPSCNTATCVLGRLSQELHRLQTYPRTNTGSNTY

SEQ ID NO: 12 - protein, 80 aa, synthetic construct
  VARIANT 2: note = AIB
  VARIANT 13: note = AIB
  DISULFID 44..50: note = Intrachain Disulfide bond
  MOD_RES 80: note = C-terminal amide
YXEGTFTSDYSIXLDKIAQKAFVQWLIAGGPSSGAPPPSGGGGKCNTATCATQRLAEFLRHSSNNFGPILPPTNVGSNTP

SEQ ID NO: 13 - protein, 76 aa, synthetic construct
  VARIANT 2: note = AIB
  VARIANT 13: note = AIB
  MOD_RES 76: note = C-terminal amide
YXEGTFTSDYSIXLDKIAQKAFVQWLIAGGPSSGAPPPSGGGGEASELSTAALGRLSAELHELATLPRTETGSGSP

SEQ ID NO: 14 - protein, 31 aa, synthetic construct
  SITE 1: note = N-acetyl, D-tyrosine
  VARIANT 2: note = AIB
  VARIANT 24: note = Norleucine Nle
  MOD_RES 31: note = C-terminal amide
YXEGTFISDYSIAXDKIRQQDFVKWLLAQKP

SEQ ID NO: 15 - protein, 39 aa, synthetic construct
  VARIANT 2: note = AIB
  VARIANT 13: note = AIB
  MOD_RES 39: note = C-terminal amide
YXEGTFTSDYSIXLDKIAQKAFVQWLIAGGPSSGAPPPS

SEQ ID NO: 16 - protein, 37 aa, synthetic construct
  DISULFID 2..7: note = Intrachain Disulfide bond
  MOD_RES 37: note = C-terminal amide
KCNTATCATQRLAEFLRHSSNNFGPILPPTNVGSNTP

SEQ ID NO: 17 - protein, 31 aa, synthetic construct
  SITE 2: note = AIB
HXEGTFTSDVSSYLEGQAAKEFIAWLVRGRG

SEQ ID NO: 18-19 - skipped (2 entries)

SEQ ID NO: 20 - protein, 75 aa, synthetic construct
  VARIANT 2: note = Aib / alpha-aminoisobutyric acid
  MOD_RES 75: note = C-terminal amide
YXEGTFTSDYSILLEEQAAREFIEWLLAGGPSKGAPPPSGGGEASELSTAALGRLSAELHELATLPRTETGSGSP

SEQ ID NO: 21 - protein, 67 aa, synthetic construct
  VARIANT 2: note = Aib / alpha-aminoisobutyric acid
  MOD_RES 67: note = C-terminal amide
YXEGTFTSDYSILLEEQAAREFIEWLLAGGPSKGEASELSTAALGRLSAELHELATLPRTETGSGSP

SEQ ID NO: 22 - protein, 75 aa, synthetic construct
  VARIANT 2: note = Aib / alpha-aminoisobutyric acid
  MOD_RES 75: note = C-terminal amide
HXHGTFTSDYSILLEEQAAREFIEWLLAGGPSKGAPPPSGGGEASELSTAALGRLSAELHELATLPRTETGSGSP

SEQ ID NO: 23 - protein, 75 aa, synthetic construct
  VARIANT 2: note = Aib / alpha-aminoisobutyric acid
  MOD_RES 75: note = C-terminal amide
YXEGTFTSDYSILLEEQAAREFIEWLLAGGPSKGAPPPSGGGGASELSTAALGRLSAELHRLATLPRTETGSGSP

SEQ ID NO: 24 - protein, 75 aa, synthetic construct
  VARIANT 2: note = Aib / alpha-aminoisobutyric acid
  MOD_RES 75: note = C-terminal amide
HXHGTFTSDYSILLEEQAAREFIEWLLAGGPSKGAPPPSGGGGASELSTAALGRLSAELHRLATLPRTETGSGSP

SEQ ID NO: 25 - protein, 75 aa, synthetic construct; identical to SEQ ID NO: 22
  VARIANT 2: note = Aib / alpha-aminoisobutyric acid
  MOD_RES 75: note = C-terminal amide

SEQ ID NO: 26 - protein, 75 aa, synthetic construct; identical to SEQ ID NO: 22
  VARIANT 2: note = Aib / alpha-aminoisobutyric acid
  MOD_RES 75: note = C-terminal amide

SEQ ID NO: 27 - protein, 67 aa, synthetic construct
  VARIANT 2: note = Aib / alpha-aminoisobutyric acid
  MOD_RES 67: note = C-terminal amide
HXHGTFTSDYSILLEEQAAREFIEWLLAGGPSKGEASELSTAALGRLSAELHRLATLPRTETGSGSP

SEQ ID NO: 28 - protein, 67 aa, synthetic construct
  VARIANT 2: note = Aib / alpha-aminoisobutyric acid
  MOD_RES 67: note = C-terminal amide
YXEGTFTSDYSILLEEQAAREFIEWLLAGGPSKGEASELSTAALGRLSAELHRLATLPRTETGSGSP

SEQ ID NO: 29 - protein, 67 aa, synthetic construct; identical to SEQ ID NO: 28
  VARIANT 2: note = Aib / alpha-aminoisobutyric acid
  MOD_RES 67: note = C-terminal amide

SEQ ID NO: 30 - protein, 66 aa, synthetic construct
  VARIANT 2: note = Aib / alpha-aminoisobutyric acid
  MOD_RES 66: note = C-terminal amide
YXEGTFTSDYSILLEEQAAREFIEWLLAGGPSKGASELSTAALGRLSAELHRLATLPRTETGSGSP

SEQ ID NO: 31 - protein, 76 aa, synthetic construct
  VARIANT 2: note = Aib / alpha-aminoisobutyric acid
  MOD_RES 76: note = C-terminal amide
YXEGTFTSDYSILLEEQAAREFIEWLLAGGPSKGALAQTLAQTLASELSTAALGRLSAELHELATLPRTETGSGSP

SEQ ID NO: 32 - protein, 80 aa, synthetic construct
  VARIANT 2: note = Aib / alpha-aminoisobutyric acid
  MOD_RES 80: note = C-terminal amide
HXHGTFTSDYSILLEEQAAREFIEWLLAGGPSKGAPPPSLAQTLAQTLASELSTAALGRLSAELHELATLPRTETGSGSP

SEQ ID NO: 33 - protein, 76 aa, synthetic construct
  VARIANT 2: note = Aib / alpha-aminoisobutyric acid
  MOD_RES 76: note = C-terminal amide
YXEGTFTSDYSILLEEQAAREFIEWLLAGGPSKGALAQTLGTNEASELSTAALGRLSAELHELATLPRTETGSGSP

SEQ ID NO: 34 - protein, 76 aa, synthetic construct
  VARIANT 2: note = Aib / alpha-aminoisobutyric acid
  MOD_RES 76: note = C-terminal amide
YXEGTFTSDYSILLEEQAAREFIEWLLAGGPSKGALAQTLFVNQASELSTAALGRLSAELHELATLPRTETGSGSP

SEQ ID NO: 35 - protein, 67 aa, synthetic construct; identical to SEQ ID NO: 27
  VARIANT 2: note = Aib / alpha-aminoisobutyric acid
  MOD_RES 67: note = C-terminal amide

SEQ ID NO: 36 - protein, 67 aa, synthetic construct; identical to SEQ ID NO: 21
  VARIANT 2: note = Aib / alpha-aminoisobutyric acid
  MOD_RES 67: note = C-terminal amide

SEQ ID NO: 37 - protein, 67 aa, synthetic construct
  VARIANT 2: note = Aib / alpha-aminoisobutyric acid
  MOD_RES 67: note = C-terminal amide
YXEGTFTSDYSILLEEQAAREFIEWLLAGGPSKGEASELSTAQLGRLSAELHRLATLPRTETGSGSP

SEQ ID NO: 38 - protein, 67 aa, synthetic construct
  VARIANT 2: note = Aib / alpha-aminoisobutyric acid
  MOD_RES 67: note = C-terminal amide
YXEGTFTSDYSILLEEQAAREFIEWLLAGGPSKGEASELSTAALARLSAELHRLATLPRTETGSGSP

SEQ ID NO: 39 - protein, 67 aa, synthetic construct
  VARIANT 2: note = Aib / alpha-aminoisobutyric acid
  MOD_RES 67: note = C-terminal amide
YXEGTFTSDYSILLEEQAAREFIEWLLAGGPSKGEASELSTAALGRLSAELHYLATLPRTETGSGSP

SEQ ID NO: 40 - protein, 75 aa, synthetic construct
  VARIANT 2: note = Aib / alpha-aminoisobutyric acid
  MOD_RES 75: note = C-terminal amide
HXHGTFTSDYSILLEEQAAREFIEWLLAGGPSKGAPPPSGGGEASELSTAALGRLSAELHRLATLPRTETGSGSP

SEQ ID NO: 41 - protein, 75 aa, synthetic construct
  VARIANT 2: note = Aib / alpha-aminoisobutyric acid
  MOD_RES 75: note = C-terminal amide
YXEGTFTSDYSILLEEQAAREFIEWLLAGGPSSKAPPPSGGGEASELSTAALGRLSAELHRLATLPRTETGSGSP

SEQ ID NO: 42 - protein, 68 aa, synthetic construct
  VARIANT 2: note = Aib / alpha-aminoisobutyric acid
  MOD_RES 68: note = C-terminal amide
YXEGTFTSDYSILLEEQAAREFIEWLLAGGPSKGAEASELSTAALGRLSAELHELATLPRTETGSGSP

SEQ ID NO: 43 - protein, 70 aa, synthetic construct
  VARIANT 2: note = Aib / alpha-aminoisobutyric acid
  MOD_RES 70: note = C-terminal amide
YXEGTFTSDYSILLEEQAAREFIEWLLAGGPSKGAPPEASELSTAALGRLSAELHELATLPRTETGSGSP

SEQ ID NO: 44 - protein, 67 aa, synthetic construct
  VARIANT 2: note = Aib / alpha-aminoisobutyric acid
  MOD_RES 67: note = C-terminal amide
YXEGTFTSDYSILLEEQAAREFIEWLLAGGPSKGEASHLSTAQTQRLSAELHELATLPRTETGSGSP

SEQ ID NO: 45 - protein, 67 aa, synthetic construct
  VARIANT 2: note = Aib / alpha-aminoisobutyric acid
  MOD_RES 67: note = C-terminal amide
YXEGTFTSDYSILLEEQAAREFIEWLLAGGPSKGEASHLSTAQTARLSAELHELATLPRTETGSGSP

SEQ ID NO: 46 - protein, 67 aa, synthetic construct
  VARIANT 2: note = Aib / alpha-aminoisobutyric acid
  MOD_RES 67: note = C-terminal amide
HXHGTFTSDYSILLEEQAAREFIEWLLAGGPSKGEASHLSTAQTARLSAELHELATLPRTETGSGSP

SEQ ID NO: 47 - protein, 75 aa, synthetic construct
  VARIANT 2: note = Aib / alpha-aminoisobutyric acid
  MOD_RES 75: note = C-terminal amide
HXHGTFTSDYSILLEEQAAREFIEWLLAGGPSKGAPPPSGGGEASHLSTAQTARLSAELHRLADLPRTETGSGSP

SEQ ID NO: 48 - protein, 76 aa, synthetic construct
  VARIANT 2: note = Aib / alpha-aminoisobutyric acid
  MOD_RES 76: note = C-terminal amide
YXEGTFTSDYSILLEEQAAREFIEWLLAGGPSKGGQAPGQAPGEASHLSTAQTQRLSAELHELATLPRTETGSGSP

SEQ ID NO: 49 - protein, 76 aa, synthetic construct
  VARIANT 2: note = Aib / alpha-aminoisobutyric acid
  MOD_RES 76: note = C-terminal amide
YXEGTFTSDYSILLEEQAAREFIEWLLAGGPSKGGQEPGQEPGEASHLSTAQTQRLSAELHELATLPRTETGSGSP

SEQ ID NO: 50 - protein, 76 aa, synthetic construct
  VARIANT 50: note = Aib / alpha-aminoisobutyric acid
  MOD_RES 76: note = C-terminal amide
YXEGTFTSDYSILLEEQAAREFIEWLLAGGPSKGGGGEGGGEGEASHLSTAQTQRLSAELHELATLPRTETGSGSP

SEQ ID NO: 51 - protein, 75 aa, synthetic construct
  VARIANT 2: note = Aib / alpha-aminoisobutyric acid
  MOD_RES 75: note = C-terminal amide
YXEGTFTSDYSILLEEQAAREFIEWLLAGGPSKGAPPPSGGGEASELSTAALGRLSAELHRLADLPRTETGSGSP

SEQ ID NO: 52 - protein, 75 aa, synthetic construct; identical to SEQ ID NO: 41
  VARIANT 2: note = Aib / alpha-aminoisobutyric acid
  MOD_RES 75: note = C-terminal amide

SEQ ID NO: 53 - protein, 67 aa, synthetic construct
  VARIANT 2: note = Aib / alpha-aminoisobutyric acid
  MOD_RES 67: note = C-terminal amide
HXHGTFTSDYSILLEEQAAREFIEWLLAGGPSKGEASQLSTAALGRLSAELHRLADLPRTETGSGSP

SEQ ID NO: 54 - protein, 76 aa, synthetic construct
  VARIANT 2: note = Aib / alpha-aminoisobutyric acid
  MOD_RES 76: note = C-terminal amide
HXHGTFTSDYSILLEEQAAREFIEWLLAGGPSKGALAQTLGTNEASELSTAALGRLSAELHRLATLPRTETGSGSP

SEQ ID NO: 55 - protein, 76 aa, synthetic construct
  VARIANT 2: note = Aib / alpha-aminoisobutyric acid
  MOD_RES 76: note = C-terminal amide
HXHGTFTSDYSILLEEQAAREFIEWLLAGGPSKGALAQTLGTNEASHLSTAQTQRLSAELHELATLPRTETGSGSP

SEQ ID NO: 56 - protein, 76 aa, synthetic construct
  VARIANT 2: note = Aib / alpha-aminoisobutyric acid
  MOD_RES 76: note = C-terminal amide
HXHGTFTSDYSILLEEQAAREFIEWLLAGGPSKGAGQAPGQAPGASHLSTAALGRLSAELHRLATLPRTETGSGSP

SEQ ID NO: 57 - protein, 76 aa, synthetic construct
  VARIANT 2: note = Aib / alpha-aminoisobutyric acid
  MOD_RES 76: note = C-terminal amide
HXHGTFTSDYSILLEEQAAREFIEWLLAGGPSKGAGQAPGQAPGASHLSTAQTQRLSAELHELATLPRTETGSGSP

SEQ ID NO: 58 - protein, 76 aa, synthetic construct
  VARIANT 2: note = Aib / alpha-aminoisobutyric acid
  MOD_RES 76: note = C-terminal amide
YXEGTFTSDYSILLEEQAAREFIEWLLAGGPSSGALAQTLAQTLASHLSTAQTQRLSAELHKLATLPRTETGSGSP

SEQ ID NO: 59 - protein, 75 aa, synthetic construct
  VARIANT 2: note = Aib / alpha-aminoisobutyric acid
  MOD_RES 75: note = C-terminal amide
YXEGTFTSDYSILLEEQAAREFIEWLLAGGPSSGAPPPSGGGEASHLSTAQTQRLSAELHKLATLPRTETGSGSP

SEQ ID NO: 60 - protein, 67 aa, synthetic construct
  VARIANT 2: note = Aib / alpha-aminoisobutyric acid
  MOD_RES 67: note = C-terminal amide
YXEGTFTSDYSILLEEQAAREFIEWLLAGGPSSGEASHLSTAQTQRLSAELHKLATLPRTETGSGSP

SEQ ID NO: 61 - protein, 76 aa, synthetic construct
  VARIANT 2: note = Aib / alpha-aminoisobutyric acid
  MOD_RES 76: note = C-terminal amide
HXHGTFTSDYSILLEEQAAREFIEWLLAGGPSSGALAQTLAQTLASHLSTAQTQRLSAELHKLATLPRTETGSGSP

SEQ ID NO: 62 - protein, 76 aa, synthetic construct
  VARIANT 2: note = Aib / alpha-aminoisobutyric acid
  MOD_RES 76: note = C-terminal amide
YXEGTFTSDYSILLEEQAAREFIEWLLAGGPSSGAGQAPGQAPGASHLSTAQTQRLSAELHKLATLPRTETGSGSP

SEQ ID NO: 63 - protein, 76 aa, synthetic construct
  VARIANT 2: note = Aib / alpha-aminoisobutyric acid
  MOD_RES 76: note = C-terminal amide
YXEGTFTSDYSILLEEQAAREFIEWLLAGGPSSGAGQAPGQAPGASHLSTAALGRLSAELHKLATLPRTETGSGSP

SEQ ID NO: 64 - protein, 76 aa, synthetic construct
  VARIANT 2: note = Aib / alpha-aminoisobutyric acid
  MOD_RES 76: note = C-terminal amide
YXEGTFTSDYSILLEEQAAREFIEWLLAGGPSSGAGQAPGQAPGASKLSTAQTQRLSAELHRLATLPRTETGSGSP

SEQ ID NO: 65 - protein, 76 aa, synthetic construct
  VARIANT 2: note = Aib / alpha-aminoisobutyric acid
  MOD_RES 76: note = C-terminal amide
YXEGTFTSDYSILLEEQAAREFIEWLLAGGPSSGAGQAPGQAPGASHLSTAQTQRLSAKLHRLATLPRTETGSGSP

SEQ ID NO: 66 - protein, 76 aa, synthetic construct
  VARIANT 2: note = Aib / alpha-aminoisobutyric acid
  MOD_RES 76: note = C-terminal amide
YXEGTFTSDYSILLEEQAAREFIEWLLAGGPSSGAGQAPGQAPGASHLSTAQTQRLSAELHRLKTLPRTETGSGSP

SEQ ID NO: 67 - protein, 76 aa, synthetic construct
  VARIANT 2: note = Aib / alpha-aminoisobutyric acid
  MOD_RES 76: note = C-terminal amide
YXEGTFTSDYSILLEEQAAREFIEWLLAGGPSSGAGQAPGQAPGASHLSTAQTQRLSAELHRLATLPKTETGSGSP

SEQ ID NO: 68 - protein, 76 aa, synthetic construct
  VARIANT 2: note = Aib / alpha-aminoisobutyric acid
  MOD_RES 76: note = C-terminal amide
YXEGTFTSDYSILLEEQAAREFIEWLLAGGPSSGAGQAPGQAPLASHLSTAQTQRLSAELHKLATLPRTETGSGSP

SEQ ID NO: 69 - protein, 76 aa, synthetic construct; identical to SEQ ID NO: 58
  VARIANT 2: note = Aib / alpha-aminoisobutyric acid
  MOD_RES 76: note = C-terminal amide

SEQ ID NO: 70 - protein, 76 aa, synthetic construct; identical to SEQ ID NO: 58
  VARIANT 2: note = Aib / alpha-aminoisobutyric acid
  MOD_RES 76: note = C-terminal amide

SEQ ID NO: 71 - protein, 75 aa, synthetic construct; identical to SEQ ID NO: 47
  VARIANT 2: note = Aib / alpha-aminoisobutyric acid
  MOD_RES 75: note = C-terminal amide

SEQ ID NO: 72 - protein, 67 aa, synthetic construct; identical to SEQ ID NO: 53
  VARIANT 2: note = Aib / alpha-aminoisobutyric acid
  MOD_RES 67: note = C-terminal amide

SEQ ID NO: 73 - protein, 67 aa, synthetic construct
  VARIANT 2: note = Aib / alpha-aminoisobutyric acid
  MOD_RES 67: note = C-terminal amide
HXHGTFTSDYSILLEEQAAREFIEWLLAGGPSKGEASQLSTAALGRLSAELHTLATLPRTETGSGSP

SEQ ID NO: 74 - protein, 67 aa, synthetic construct
  VARIANT 2: note = Aib / alpha-aminoisobutyric acid
  MOD_RES 67: note = C-terminal amide
HXHGTFTSDYSILLEEQAAREFIEWLLAGGPSKGEASQLSTAALGRLSAELHQLATLPRTETGSGSP

SEQ ID NO: 75 - protein, 67 aa, synthetic construct
  VARIANT 2: note = Aib / alpha-aminoisobutyric acid
  MOD_RES 67: note = C-terminal amide
HXHGTFTSDYSILLEEQAAREFIEWLLAGGPSKGEASQLSTAALGRLSAELHGLATLPRTETGSGSP

SEQ ID NO: 76 - protein, 67 aa, synthetic construct
  VARIANT 2: note = Aib / alpha-aminoisobutyric acid
  MOD_RES 67: note = C-terminal amide
HXHGTFTSDYSILLEEQAAREFIEWLLAGGPSKGEASQLSTAALGRLSAELHHLATLPRTETGSGSP

SEQ ID NO: 77 - protein, 67 aa, synthetic construct
  VARIANT 2: note = Aib / alpha-aminoisobutyric acid
  MOD_RES 67: note = C-terminal amide
YXEGTFTSDYSILLEEQAAREFIEWLLAGGPSRGEASELSTAALGRLSAELHKLATLPRTETGSGSP

SEQ ID NO: 78 - protein, 67 aa, synthetic construct
  VARIANT 2: note = Aib / alpha-aminoisobutyric acid
  MOD_RES 67: note = C-terminal amide
YXEGTFTSDYSILLEEQAAREFIEWLLAGGPSRGEASHLSTAQTQRLSAELHKLATLPRTETGSGSP

SEQ ID NO: 79 - protein, 67 aa, synthetic construct
  VARIANT 2: note = Aib / alpha-aminoisobutyric acid
  MOD_RES 67: note = C-terminal amide
HXHGTFTSDYSILLEEQAAREFIEWLLAGGPSRGEASHLSTAQTQRLSAELHKLATLPRTETGSGSP

SEQ ID NO: 80 - protein, 75 aa, synthetic construct
  VARIANT 2: note = Aib / alpha-aminoisobutyric acid
  MOD_RES 75: note = C-terminal amide
HXHGTFTSDYSILLEEQAAREFIEWLLAGGPSRGAPPPSGGGEASELSTAALGRLSAELHKLATLPRTETGSGSP

SEQ ID NO: 81 - protein, 75 aa, synthetic construct
  VARIANT 2: note = Aib / alpha-aminoisobutyric acid
  MOD_RES 75: note = C-terminal amide
HXHGTFTSDYSILLEEQAAREFIEWLLAGGPSRGAPPPSGGGEASHLSTQTQGRLSAELHKLATLPRTETGSGSP

SEQ ID NO: 82 - protein, 75 aa, synthetic construct
  VARIANT 2: note = Aib / alpha-aminoisobutyric acid
  MOD_RES 75: note = C-terminal amide
YXEGTFTSDYSILLEEQAAREFIEWLLAGGPSRGAPPPSGGGEASHLSTQTQGRLSAELHKLATLPRTETGSGSP

SEQ ID NO: 83 - protein, 67 aa, synthetic construct
  VARIANT 2: note = Aib / alpha-aminoisobutyric acid
  MOD_RES 67: note = C-terminal amide
HXHGTFTSDYSILLEEQAAREFIEWLLAGGPSKGEASQLSTAALGRLSAELHRLATLPRTETGSGSP

SEQ ID NO: 84 - protein, 67 aa, synthetic construct
  VARIANT 2: note = Aib / alpha-aminoisobutyric acid
  MOD_RES 67: note = C-terminal amide
YXEGTFTSDYSILLEEQAAREFIEWLLAGGPSGGEASHLSTAQTQRLSAELHKLATLPRTETGSGSP

SEQ ID NO: 85 - protein, 67 aa, synthetic construct
  VARIANT 2: note = Aib / alpha-aminoisobutyric acid
  MOD_RES 67: note = C-terminal amide
HXHGTFTSDYSILLEEQAAREFIEWLLAGGPSGGEASHLSTAQTQRLSAELHKLATLPRTETGSGSP

SEQ ID NO: 86 - protein, 76 aa, synthetic construct; identical to SEQ ID NO: 67
  VARIANT 2: note = Aib / alpha-aminoisobutyric acid
  MOD_RES 76: note = C-terminal amide

SEQ ID NO: 87 - protein, 76 aa, synthetic construct; identical to SEQ ID NO: 68
  VARIANT 2: note = Aib / alpha-aminoisobutyric acid
  MOD_RES 76: note = C-terminal amide

SEQ ID NO: 88 - protein, 76 aa, synthetic construct
  VARIANT 2: note = Aib / alpha-aminoisobutyric acid
  MOD_RES 76: note = C-terminal amide
HXHGTFTSDYSILLEEQAAREFIEWLLAGGPSSGAGQAPGQAPGASHLSTAQTQRLSAKLHRLATLPRTETGSGSP

SEQ ID NO: 89 - protein, 67 aa, synthetic construct
  VARIANT 2: note = Aib / alpha-aminoisobutyric acid
  MOD_RES 67: note = C-terminal amide
YXEGTFTSDYSILLEEQAAREFIEWLLAGGPSSGEASHLSTAQTQRLSAKLHRLATLPRTETGSGSP

SEQ ID NO: 90 - protein, 67 aa, synthetic construct
  VARIANT 2: note = Aib / alpha-aminoisobutyric acid
  MOD_RES 67: note = C-terminal amide
YXEGTFTSDYSILLEEQAAREFIEWLLAGGPSSGEASHLSTAQTQRLSAELHRLKTLPRTETGSGSP

SEQ ID NO: 91 - protein, 67 aa, synthetic construct
  VARIANT 2: note = Aib / alpha-aminoisobutyric acid
  MOD_RES 67: note = C-terminal amide
YXEGTFTSDYSILLEEQAAREFIEWLLAGGPSSGEASHLSTAQTQRLSAELHRLATLPKTETGSGSP

SEQ ID NO: 92 - protein, 67 aa, synthetic construct
  VARIANT 2: note = Aib / alpha-aminoisobutyric acid
  MOD_RES 67: note = C-terminal amide
HXHGTFTSDYSILLEEQAAREFIEWLLAGGPSSGEASHLSTAQTQRLSAKLHRLATLPRTETGSGSP

SEQ ID NO: 93 - protein, 67 aa, synthetic construct
  VARIANT 2: note = Aib / alpha-aminoisobutyric acid
  MOD_RES 67: note = C-terminal amide
HXHGTFTSDYSILLEEQAAREFIEWLLAGGPSSGEASHLSTAQTQRLSAELHRLKTLPRTETGSGSP

SEQ ID NO: 94 - protein, 67 aa, synthetic construct
  VARIANT 2: note = Aib / alpha-aminoisobutyric acid
  MOD_RES 67: note = C-terminal amide
HXHGTFTSDYSILLEEQAAREFIEWLLAGGPSSGEASHLSTAQTQRLSAELHRLATLPKTETGSGSP

SEQ ID NO: 95 - protein, 67 aa, synthetic construct
  VARIANT 2: note = Aib / alpha-aminoisobutyric acid
  MOD_RES 67: note = C-terminal amide
HXHGTFTSDYSILLEEQAAREFIEWLLAGGPSSGEASHLSTAQTQRLSAELHKLATLPRTETGSGAP

SEQ ID NO: 96 - protein, 74 aa, synthetic construct
  VARIANT 2: note = Aib / alpha-aminoisobutyric acid
  MOD_RES 74: note = C-terminal amide
HXHGTFTSDYSILLEEQAAREFIEWLLAGGPSKGAPPPSGGGASELSTAALGRLSAELHRLATLPRTETGSGSP

SEQ ID NO: 97 - protein, 67 aa, synthetic construct
  VARIANT 2: note = Aib / alpha-aminoisobutyric acid
  MOD_RES 67: note = C-terminal amide
HXHGTFTSDYSILLEEQAAREFIEWLLAGGPSSGEASHLSTAALGRLSAKLHRLATLPRTETGSGSP

SEQ ID NO: 98 - protein, 67 aa, synthetic construct
  VARIANT 2: note = Aib / alpha-aminoisobutyric acid
  MOD_RES 67: note = C-terminal amide
HXHGTFTSDYSILLEEQAAREFIEWLLAGGPSSGEASHLSTAALGRLSAELHKLATLPRTETGSGSP

SEQ ID NO: 99 - protein, 67 aa, synthetic construct
  VARIANT 2: note = Aib / alpha-aminoisobutyric acid
  MOD_RES 67: note = C-terminal amide
HXHGTFTSDYSILLEEQAAREFIEWLLAGGPSSGEASHLSTAALGRLSAELHRLKTLPRTETGSGSP

SEQ ID NO: 100 - protein, 67 aa, synthetic construct
  VARIANT 2: note = Aib / alpha-aminoisobutyric acid
  MOD_RES 67: note = C-terminal amide
HXHGTFTSDYSILLEEQAAREFIEWLLAGGPSSGEASHLSTAALGRLSAELHRLATLPKTETGSGSP

SEQ ID NO: 101 - protein, 76 aa, synthetic construct
  VARIANT 2: note = Aib / alpha-aminoisobutyric acid
  MOD_RES 76: note = C-terminal amide
HXHGTFTSDYSILLEEQAAREFIEWLLAGGPSSGAGQAPGQAPGASHLSTAQTQRLSAELHRLKTLPRTETGSGSP

SEQ ID NO: 102 - protein, 76 aa, synthetic construct
  VARIANT 2: note = Aib / alpha-aminoisobutyric acid
  MOD_RES 76: note = C-terminal amide
HXHGTFTSDYSILLEEQAAREFIEWLLAGGPSSGAGQAPGQAPGASHLSTAQTQRLSAELHRLATLPKTETGSGSP

SEQ ID NO: 103 - protein, 76 aa, synthetic construct
  VARIANT 2: note = Aib / alpha-aminoisobutyric acid
  MOD_RES 76: note = C-terminal amide
HXHGTFTSDYSILLEEQAAREFIEWLLAGGPSSGAGQAPGQAPLASHLSTAQTQRLSAELHKLATLPRTETGSGSP

SEQ ID NO: 104 - protein, 67 aa, synthetic construct; identical to SEQ ID NO: 78
  VARIANT 2: note = Aib / alpha-aminoisobutyric acid
  MOD_RES 67: note = C-terminal amide

SEQ ID NO: 105 - protein, 69 aa, synthetic construct
  VARIANT 2: note = Aib / alpha-aminoisobutyric acid
  MOD_RES 69: note = C-terminal amide
YXEGTFTSDYSILLEEQAAREFIEWLLAGGPSGGGGEASHLSTAQTQRLSAELHKLATLPRTETGSGSP

SEQ ID NO: 106 - protein, 69 aa, synthetic construct; identical to SEQ ID NO: 105
  VARIANT 2: note = Aib / alpha-aminoisobutyric acid
  MOD_RES 69: note = C-terminal amide

SEQ ID NO: 107 - protein, 72 aa, synthetic construct
  VARIANT 2: note = Aib / alpha-aminoisobutyric acid
  MOD_RES 72: note = C-terminal amide
YXEGTFTSDYSILLEEQAAREFIEWLLAGGPSSGAGQAPGASHLSTAQTQRLSAELHKLATLPRTETGSGSP

SEQ ID NO: 108 - protein, 76 aa, synthetic construct
  VARIANT 2: note = Aib / alpha-aminoisobutyric acid
  MOD_RES 76: note = C-terminal amide
YXEGTFTSDYSILLEEQAAREFIEWLLAGGPSSGAGQAPGQAPLASHLSTAQTARLSAELHKLATLPRTETGSGSP

SEQ ID NO: 109 - protein, 76 aa, synthetic construct
  VARIANT 2: note = Aib / alpha-aminoisobutyric acid
  MOD_RES 76: note = C-terminal amide
YXEGTFTSDYSILLEEQAAREFIEWLLAGGPSSGALQAPGQAPLASHLSTAQTQRLSAELHKLATLPRTETGSGSP

SEQ ID NO: 110 - protein, 75 aa, synthetic construct; identical to SEQ ID NO: 47
  VARIANT 2: note = Aib / alpha-aminoisobutyric acid
  MOD_RES 75: note = C-terminal amide

SEQ ID NO: 111 - protein, 75 aa, synthetic construct
  VARIANT 2: note = Aib / alpha-aminoisobutyric acid
  MOD_RES 75: note = C-terminal amide
HXHGTFTSDYSILLEEQAAREFIEWLLAGGPSKGAPPPSGGGEASHLSTAQTARLSAELHQLATLPRTETGSGSP

SEQ ID NO: 112 - protein, 76 aa, synthetic construct
  VARIANT 2: note = Aib / alpha-aminoisobutyric acid
  MOD_RES 76: note = C-terminal amide
YXEGTFTSDYSILLEEQAAREFIEWLLAGGPSSGAGQAPGQAPGASHLSTAQTQRLSAELHQLKTLPRTETGSGSP

SEQ ID NO: 113 - protein, 76 aa, synthetic construct
  VARIANT 2: note = Aib / alpha-aminoisobutyric acid
  MOD_RES 76: note = C-terminal amide
HXHGTFTSDYSILLEEQAAREFIEWLLAGGPSSGAGQAPGQAPGASHLSTAQTARLSAELHKLATLPRTETGSGSP

SEQ ID NO: 114 - protein, 76 aa, synthetic construct; identical to SEQ ID NO: 113
  VARIANT 2: note = Aib / alpha-aminoisobutyric acid
  MOD_RES 76: note = C-terminal amide

SEQ ID NO: 115 - protein, 76 aa, synthetic construct
  VARIANT 2: note = Aib / alpha-aminoisobutyric acid
  MOD_RES 76: note = C-terminal amide
YXEGTFTSDYSILLEEQAAREFIEWLLAGGPSSGALQAPGQAPLASHLSTAQTARLSAELHKLATLPRTETGSGSP

SEQ ID NO: 116 - protein, 76 aa, synthetic construct
  VARIANT 2: note = Aib / alpha-aminoisobutyric acid
  MOD_RES 76: note = C-terminal amide
HXHGTFTSDYSILLEEQAAREFIEWLLAGGPSSGALQAPGQAPGASHLSTAQTARLSAELHRLKTLPRTETGSGSP

SEQ ID NO: 117 - protein, 76 aa, synthetic construct
  VARIANT 2: note = Aib / alpha-aminoisobutyric acid
  MOD_RES 76: note = C-terminal amide
HXHGTFTSDYSILLEEQAAREFIEWLLAGGPSSGALQAPGQAPGASHLSTAQTARLSAELHRLATLPKTETGSGSP

SEQ ID NO: 118 - protein, 76 aa, synthetic construct
  VARIANT 2: note = Aib / alpha-aminoisobutyric acid
  MOD_RES 76: note = C-terminal amide
HXHGTFTSDYSILLEEQAAREFIEWLLAGGPSSGALQAPGQAPLASHLSTAQTARLSAELHKLATLPRTETGSGSP

SEQ ID NO: 119 - protein, 75 aa, synthetic construct
  VARIANT 2: note = Aib / alpha-aminoisobutyric acid
  MOD_RES 75: note = C-terminal amide
YXEGTFTSDYSILLEEQAAREFIEWLLAGGPSSGAPPPSGGGEASHLSTAQTQRLSAKLHRLATLPRTETGSGSP

SEQ ID NO: 120 - protein, 75 aa, synthetic construct; identical to SEQ ID NO: 119
  VARIANT 2: note = Aib / alpha-aminoisobutyric acid
  MOD_RES 75: note = C-terminal amide

SEQ ID NO: 121 - protein, 76 aa, synthetic construct; identical to SEQ ID NO: 66
  VARIANT 2: note = Aib / alpha-aminoisobutyric acid
  MOD_RES 76: note = C-terminal amide

SEQ ID NO: 122 - protein, 75 aa, synthetic construct; identical to SEQ ID NO: 47
  VARIANT 2: note = Aib / alpha-aminoisobutyric acid
  MOD_RES 75: note = C-terminal amide

SEQ ID NO: 123 - protein, 76 aa, synthetic construct
  VARIANT 2: note = Aib / alpha-aminoisobutyric acid
  MOD_RES 76: note = C-terminal amide
YXEGTFTSDYSILLEEQAAREFIEWLLAGGPSSGAGQAPGQAPGASHLSTAQTARLSAELHKLATLPRTETGSGSP

SEQ ID NO: 124 - protein, 76 aa, synthetic construct
  VARIANT 2: note = Aib / alpha-aminoisobutyric acid
  MOD_RES 76: note = C-terminal amide
YXEGTFTSDYSILLEEQAAREFIEWLLAGGPSSGAGQAPGQAPGASHLSTAALARLSAELHKLATLPRTETGSGSP

SEQ ID NO: 125 - protein, 4 aa, synthetic construct
APPE

SEQ ID NO: 126 - protein, 4 aa, synthetic construct
GGGE

SEQ ID NO: 127 - protein, 6 aa, synthetic construct
AGQAPG

SEQ ID NO: 128 - protein, 8 aa, synthetic construct
APPPSGGG

SEQ ID NO: 129 - protein, 9 aa, synthetic construct
APPPSGGGE

SEQ ID NO: 130 - protein, 9 aa, synthetic construct
APPPSGGGG

SEQ ID NO: 131 - protein, 10 aa, synthetic construct
ALAQTLAQTL

SEQ ID NO: 132 - protein, 10 aa, synthetic construct
ALAQTLFVNQ

SEQ ID NO: 133 - protein, 10 aa, synthetic construct
ALAQTLGTNE

SEQ ID NO: 134 - protein, 10 aa, synthetic construct
ALQAPGQAPG

SEQ ID NO: 135 - protein, 10 aa, synthetic construct
ALQAPGQAPL

SEQ ID NO: 136 - protein, 10 aa, synthetic construct
AGQAPGQAPG

SEQ ID NO: 137 - protein, 10 aa, synthetic construct
AGQAPGQAPL

SEQ ID NO: 138 - protein, 10 aa, synthetic construct
GGGEGGGEGE

SEQ ID NO: 139 - protein, 10 aa, synthetic construct
GQAPGQAPGE

SEQ ID NO: 140 - protein, 10 aa, synthetic construct
GQEPGQEPGE

SEQ ID NO: 141 - protein, 14 aa, synthetic construct
APPPSLAQTLAQTL

SEQ ID NO: 142 - protein, 5 aa, synthetic construct
AGGGG

SEQ ID NO: 143 - protein, 10 aa, synthetic construct
AGEAPGQAPG

SEQ ID NO: 144 - protein, 10 aa, synthetic construct
AGEAPGEAPG

SEQ ID NO: 145 - protein, 10 aa, synthetic construct
AGQAPGQAPA

SEQ ID NO: 146 - protein, 10 aa, synthetic construct
AGQAPGQAPE

SEQ ID NO: 147 - protein, 10 aa, synthetic construct
AGQAPGQAPP

SEQ ID NO: 148 - protein, 10 aa, synthetic construct
AGQAPGQAPS

SEQ ID NO: 149 - protein, 10 aa, synthetic construct
AGQAPGQAPV

SEQ ID NO: 150 - protein, 10 aa, synthetic construct
EGQAPGQAPG

SEQ ID NO: 151 - protein, 10 aa, synthetic construct
AGQEPGQAPG

SEQ ID NO: 152 - protein, 10 aa, synthetic construct
AGQAEGQAPG

SEQ ID NO: 153 - protein, 10 aa, synthetic construct
AGQAPEQAPG

SEQ ID NO: 154 - protein, 10 aa, synthetic construct
AGQAPGEAPG

SEQ ID NO: 155 - protein, 10 aa, synthetic construct
AGQAPGQEPG

SEQ ID NO: 156 - protein, 10 aa, synthetic construct
AGQAPGQAEG

SEQ ID NO: 157 - protein, 10 aa, synthetic construct
AGQEPGQEPG

SEQ ID NO: 158 - protein, 9 aa, synthetic construct
AGQAPGQAP

SEQ ID NO: 159 - protein, 10 aa, synthetic construct
AGQAPGEAPL

SEQ ID NO: 160 - skipped

SEQ ID NO: 161 - protein, 34 aa, synthetic construct
  VARIANT 1: note = H, Y
  VARIANT 2: note = Aib / alpha-aminoisobutyric acid
  VARIANT 3: note = E, H
  VARIANT 12: note = I, K
  VARIANT 17: note = Q, I
  VARIANT 20: note = R, Q
  VARIANT 24: note = A, E, Q
  VARIANT 27: note = J
  VARIANT 28: note = A, Q
  VARIANT 33: note = R, G, K, S
  VARIANT 34: note = G, E, K
XXXGTFTSDYSXLLEEXAAXEFIXWLXXGGPSXX

SEQ ID NO: 162 - protein, 34 aa, synthetic construct
  VARIANT 2: note = Aib / alpha-aminoisobutyric acid
  VARIANT 12: note = I, K
  VARIANT 20: note = R, Q
  VARIANT 24: note = A, E, Q
  VARIANT 27: note = J
  VARIANT 28: note = A, Q
  VARIANT 34: note = G, E
YXEGTFTSDYSXLLEEIAAXEFIXWLXXGGPSSX

SEQ ID NO: 163 - protein, 34 aa, synthetic construct
  VARIANT 2: note = Aib / alpha-aminoisobutyric acid
  VARIANT 12: note = I, K
YXEGTFTSDYSXLLEEIAAREFIEWLLAGGPSSG

SEQ ID NO: 164 - protein, 32 aa, synthetic construct
  VARIANT 2: note = G, S
  VARIANT 3: note = Q, E, H, K
  VARIANT 8: note = A, Q
  VARIANT 9: note = Q, L, T
  VARIANT 10: note = A, G, Q
  VARIANT 15: note = E, K
  VARIANT 18: note = R, Q, E, G, H, K, T, Y
  VARIANT 20: note = A, K
  VARIANT 21: note = D, T
  VARIANT 22: note = L, E
  VARIANT 24: note = R, K
  VARIANT 31: note = A, S
AXXLSTAXXXRLSAXLHXLXXXPXTETGSGXP

SEQ ID NO: 165 - protein, 32 aa, synthetic construct
  VARIANT 2: note = G, S
  VARIANT 3: note = Q, E, H
  VARIANT 8: note = A, Q
  VARIANT 9: note = L, T
  VARIANT 10: note = A, G, Q
  VARIANT 18: note = Q, E, K
  VARIANT 22: note = L, E
AXXLSTAXXXRLSAELHXLATXPRTETGSGSP

SEQ ID NO: 166 - protein, 32 aa, synthetic construct
  VARIANT 3: note = E, H
ASXLSTAQTQRLSAELHKLATLPRTETGSGSP

SEQ ID NO: 167-169 - skipped (3 entries)

SEQ ID NO: 170 - protein, 76 aa, synthetic construct
  VARIANT 2: note = Aib / alpha-aminoisobutyric acid
  MOD_RES 76: note = C-terminal amide
YXEGTFTSDYSILLEEIAAREFIEWLLAGGPSSGAGQAPGQAPGASHLSTAQTQRLSAELHKLATLPRTETGSGSP

SEQ ID NO: 171 - protein, 76 aa, synthetic construct
  VARIANT 2: note = Aib / alpha-aminoisobutyric acid
  MOD_RES 76: note = C-terminal amide
YXEGTFTSDYSILLEEIAAREFIEWLIAGGPSSGAGQAPGQAPGASHLSTAQTQRLSAELHKLATLPRTETGSGSP

SEQ ID NO: 172 - protein, 76 aa, synthetic construct
  VARIANT 2: note = Aib / alpha-aminoisobutyric acid
  MOD_RES 76: note = C-terminal amide
YXEGTFTSDYSKLLEEIAAREFIEWLLAGGPSSGAGQAPGQAPGASHLSTAQLGRLSAELHELATLPRTETGSGSP

SEQ ID NO: 173 - protein, 76 aa, synthetic construct
  VARIANT 2: note = Aib / alpha-aminoisobutyric acid
  MOD_RES 76: note = C-terminal amide
YXEGTFTSDYSKLLEEIAAREFIEWLLAGGPSSGAGQAPGQAPGASHLSTAQLGRLSAELHQLATLPRTETGSGSP

SEQ ID NO: 174 - protein, 76 aa, synthetic construct
  VARIANT 2: note = Aib / alpha-aminoisobutyric acid
  MOD_RES 76: note = C-terminal amide
YXEGTFTSDYSKLLEEIAAREFIEWLLAGGPSSGAGQAPGQAPGASHLSTAQTQRLSAELHELATLPRTETGSGSP

SEQ ID NO: 175 - protein, 76 aa, synthetic construct
  VARIANT 2: note = Aib / alpha-aminoisobutyric acid
  MOD_RES 76: note = C-terminal amide
YXEGTFTSDYSILLEEIAAREFIEWLLAGGPSSGAGQAPGQAPLASHLSTAQTQRLSAELHKLATLPRTETGSGSP

SEQ ID NO: 176 - protein, 76 aa, synthetic construct
  VARIANT 2: note = Aib / alpha-aminoisobutyric acid
  MOD_RES 76: note = C-terminal amide
YXEGTFTSDYSKLLEEIAAREFIEWLLAGGPSSGAGQAPGQAPGASHLSTAALGRLSAELHELATLPRTETGSGSP

SEQ ID NO: 177 - protein, 76 aa, synthetic construct
  VARIANT 2: note = Aib / alpha-aminoisobutyric acid
  MOD_RES 76: note = C-terminal amide
YXEGTFTSDYSKLLEEIAAREFIEWLIAGGPSSGAGQAPGQAPGASHLSTAALGRLSAELHELATLPRTETGSGSP

SEQ ID NO: 178 - protein, 76 aa, synthetic construct
  VARIANT 2: note = Aib / alpha-aminoisobutyric acid
  MOD_RES 76: note = C-terminal amide
YXEGTFTSDYSILLEEIAAREFIEWLLAGGPSSGAGQAPGQAPLASHLSTAQTARLSAELHKLATLPRTETGSGSP

SEQ ID NO: 179 - protein, 76 aa, synthetic construct
  VARIANT 2: note = Aib / alpha-aminoisobutyric acid
  MOD_RES 76: note = C-terminal amide
YXEGTFTSDYSKLLEEIAAREFIEWLLAGGPSSGAGQAPGQAPLASHLSTAQLGRLSAELHQLATLPRTETGSGSP

SEQ ID NO: 180 - protein, 68 aa, synthetic construct
  VARIANT 2: note = Aib / alpha-aminoisobutyric acid
  MOD_RES 68: note = C-terminal amide
YXEGTFTSDYSKLLEEIAAREFIEWLLAGGPSSGAGASHLSTAQLGRLSAELHQLATLPRTETGSGSP

SEQ ID NO: 181 - protein, 68 aa, synthetic construct
  VARIANT 2: note = Aib / alpha-aminoisobutyric acid
  MOD_RES 68: note = C-terminal amide
YXEGTFTSDYSILLEEIAAREFIEWLLAGGPSSGAGASHLSTAQTQRLSAELHKLATLPRTETGSGSP

SEQ ID NO: 182 - protein, 76 aa, synthetic construct
  VARIANT 2: note = Aib / alpha-aminoisobutyric acid
  MOD_RES 76: note = C-terminal amide
YXEGTFTSDYSILLEEIAAREFIEWLLAGGPSSGAGQAPGQAPLASQLSTAQTQRLSAELHKLATLPRTETGSGSP

SEQ ID NO: 183 - protein, 76 aa, synthetic construct
  VARIANT 2: note = Aib / alpha-aminoisobutyric acid
  MOD_RES 76: note = C-terminal amide
YXEGTFTSDYSILLEEIAAREFIEWLLAGGPSSGAGQAPGQAPLASELSTAQTQRLSAELHKLATLPRTETGSGSP

SEQ ID NO: 184 - protein, 67 aa, synthetic construct
  VARIANT 2: note = Aib / alpha-aminoisobutyric acid
  MOD_RES 67: note = C-terminal amide
YXEGTFTSDYSILLEEIAAREFIEWLLAGGPSSGEASHLSTAQTQRLSAELHKLATLPRTETGSGSP

SEQ ID NO: 185 - protein, 76 aa, synthetic construct
  VARIANT 2: note = Aib / alpha-aminoisobutyric acid
  MOD_RES 76: note = C-terminal amide
YXEGTFTSDYSILLEEIAAREFIEWLLAGGPSSGAGQAPGQAPAASHLSTAQTQRLSAELHKLATLPRTETGSGSP

SEQ ID NO: 186 - protein, 76 aa, synthetic construct
  VARIANT 2: note = Aib / alpha-aminoisobutyric acid
  MOD_RES 76: note = C-terminal amide
YXEGTFTSDYSILLEEIAAREFIEWLLAGGPSSGAGQAPGQAPEASHLSTAQTQRLSAELHKLATLPRTETGSGSP

SEQ ID NO: 187 - protein, 76 aa, synthetic construct
  VARIANT 2: note = Aib / alpha-aminoisobutyric acid
  MOD_RES 76: note = C-terminal amide
YXEGTFTSDYSILLEEIAAREFIEWLLAGGPSSGAGQAPGQAPPASHLSTAQTQRLSAELHKLATLPRTETGSGSP

SEQ ID NO: 188 - protein, 76 aa, synthetic construct
  VARIANT 2: note = Aib / alpha-aminoisobutyric acid
  MOD_RES 76: note = C-terminal amide
YXEGTFTSDYSILLEEIAAREFIEWLLAGGPSSGAGQAPGQAPSASHLSTAQTQRLSAELHKLATLPRTETGSGSP

SEQ ID NO: 189 - protein, 76 aa, synthetic construct
  VARIANT 2: note = Aib / alpha-aminoisobutyric acid
  MOD_RES 76: note = C-terminal amide
YXEGTFTSDYSILLEEIAAREFIEWLLAGGPSSGAGQAPGQAPVASHLSTAQTQRLSAELHKLATLPRTETGSGSP

SEQ ID NO: 190 - protein, 71 aa, synthetic construct
  VARIANT 2: note = Aib / alpha-aminoisobutyric acid
  MOD_RES 71: note = C-terminal amide
YXEGTFTSDYSILLEEIAAREFIEWLLAGGPSSGAGGGGASHLSTAQTQRLSAELHKLATLPRTETGSGSP

SEQ ID NO: 191 - protein, 71 aa, synthetic construct
  VARIANT 2: note = Aib / alpha-aminoisobutyric acid
  MOD_RES 71: note = C-terminal amide
YXEGTFTSDYSILLEEIAAREFIEWLLAGGPSSGAGGGGAGELSTAQTQRLSAELHKLATLPRTETGSGSP

SEQ ID NO: 192 - protein, 76 aa, synthetic construct
  VARIANT 2: note = Aib / alpha-aminoisobutyric acid
  MOD_RES 76: note = C-terminal amide
YXEGTFTSDYSILLEEIAAREFIEWLIAGGPSSGAGQAPGQAPLASHLSTAQTQRLSAELHKLATLPRTETGSGSP

SEQ ID NO: 193 - protein, 76 aa, synthetic construct
  VARIANT 2: note = Aib / alpha-aminoisobutyric acid
  MOD_RES 76: note = C-terminal amide
YXEGTFTSDYSILLEEIAAREFIEWLLAGGPSSGAGQAPGQAPGASELSTAQTQRLSAELHKLATLPRTETGSGSP

SEQ ID NO: 194 - protein, 76 aa, synthetic construct
  VARIANT 2: note = Aib / alpha-aminoisobutyric acid
  MOD_RES 76: note = C-terminal amide
YXEGTFTSDYSILLEEIAAREFIEWLLAGGPSSGAGQAPGQAPGASELSTAQTARLSAELHKLATLPRTETGSGSP

SEQ ID NO: 195 - protein, 76 aa, synthetic construct
  VARIANT 2: note = Aib / alpha-aminoisobutyric acid
  MOD_RES 76: note = C-terminal amide
YXEGTFTSDYSILLEEIAAREFIEWLLAGGPSSEAGQAPGQAPGASHLSTAQTQRLSAELHKLATLPRTETGSGSP

SEQ ID NO: 196 - protein, 76 aa, synthetic construct
  VARIANT 2: note = Aib / alpha-aminoisobutyric acid
  MOD_RES 76: note = C-terminal amide
YXEGTFTSDYSILLEEIAAREFIEWLLAGGPSSGEGQAPGQAPGASHLSTAQTQRLSAELHKLATLPRTETGSGSP

SEQ ID NO: 197 - protein, 76 aa, synthetic construct
  VARIANT 2: note = Aib / alpha-aminoisobutyric acid
  MOD_RES 76: note = C-terminal amide
YXEGTFTSDYSILLEEIAAREFIEWLLAGGPSSGAGEAPGQAPGASHLSTAQTQRLSAELHKLATLPRTETGSGSP

SEQ ID NO: 198 - protein, 76 aa, synthetic construct
  VARIANT 2: note = Aib / alpha-aminoisobutyric acid
  MOD_RES 76: note = C-terminal amide
YXEGTFTSDYSILLEEIAAREFIEWLLAGGPSSGAGQEPGQAPGASHLSTAQTQRLSAELHKLATLPRTETGSGSP

SEQ ID NO: 199 - protein, 76 aa, synthetic construct
  VARIANT 2: note = Aib / alpha-aminoisobutyric acid
  MOD_RES 76: note = C-terminal amide
YXEGTFTSDYSILLEEIAAREFIEWLLAGGPSSGAGQAEGQAPGASHLSTAQTQRLSAELHKLATLPRTETGSGSP

SEQ ID NO: 200 - protein, 76 aa, synthetic construct
  VARIANT 2: note = Aib / alpha-aminoisobutyric acid
  MOD_RES 76: note = C-terminal amide
YXEGTFTSDYSILLEEIAAREFIEWLLAGGPSSGAGQAPEQAPGASHLSTAQTQRLSAELHKLATLPRTETGSGSP

SEQ ID NO: 201 - protein, 76 aa, synthetic construct
  VARIANT 2: note = Aib / alpha-aminoisobutyric acid
  MOD_RES 76: note = C-terminal amide
YXEGTFTSDYSILLEEIAAREFIEWLLAGGPSSGAGQAPGEAPGASHLSTAQTQRLSAELHKLATLPRTETGSGSP

SEQ ID NO: 202 - protein, 76 aa, synthetic construct
  VARIANT 2: note = Aib / alpha-aminoisobutyric acid
  MOD_RES 76: note = C-terminal amide
YXEGTFTSDYSILLEEIAAREFIEWLLAGGPSSGAGQAPGQEPGASHLSTAQTQRLSAELHKLATLPRTETGSGSP

SEQ ID NO: 203 - protein, 76 aa, synthetic construct
  VARIANT 2: note = Aib / alpha-aminoisobutyric acid
  MOD_RES 76: note = C-terminal amide
YXEGTFTSDYSILLEEIAAREFIEWLLAGGPSSGAGQAPGQAEGASHLSTAQTQRLSAELHKLATLPRTETGSGSP

SEQ ID NO: 204 - protein, 76 aa, synthetic construct
  VARIANT 2: note = Aib / alpha-aminoisobutyric acid
  MOD_RES 76: note = C-terminal amide
YXEGTFTSDYSILLEEIAAREFIEWLIAGGPSSGAGQEPGQEPGASHLSTAQTQRLSAELHKLATLPRTETGSGSP

SEQ ID NO: 205 - protein, 76 aa, synthetic construct
  VARIANT 2: note = Aib / alpha-aminoisobutyric acid
  MOD_RES 76: note = C-terminal amide
YXEGTFTSDYSILLEEIAAREFIAWLIAGGPSSGAGQEPGQEPGASHLSTAQTQRLSAELHKLATLPRTETGSGSP

SEQ ID NO: 206 - protein, 76 aa, synthetic construct
  VARIANT 2: note = Aib / alpha-aminoisobutyric acid
  MOD_RES 76: note = C-terminal amide
YXEGTFTSDYSILLEEIAAREFIQWLIAGGPSSGAGQEPGQEPGASHLSTAQTQRLSAELHKLATLPRTETGSGSP

SEQ ID NO: 207 - protein, 68 aa, synthetic construct
  VARIANT 2: note = Aib / alpha-aminoisobutyric acid
  MOD_RES 68: note = C-terminal amide
YXEGTFTSDYSILLEEIAAREFIEWLIAGGPSSGAGASHLSTAQTQRLSAELHKLATLPRTETGSGSP

SEQ ID NO: 208 - protein, 75 aa, synthetic construct
  VARIANT 2: note = Aib / alpha-aminoisobutyric acid
  MOD_RES 75: note = C-terminal amide
YXEGTFTSDYSILLEEIAAREFIEWLLAGGPSSGAGQAPGQAPASHLSTAQTQRLSAELHKLATLPRTETGSGSP

SEQ ID NO: 209 - protein, 76 aa, synthetic construct
  VARIANT 2: note = Aib / alpha-aminoisobutyric acid
  MOD_RES 76: note = C-terminal amide
YXEGTFTSDYSILLEEIAAREFIEWLLAGGPSSGAGQAPGQAPGASHLSTAQTQRLSAELHKLATEPRTETGSGSP

SEQ ID NO: 210 - protein, 76 aa, synthetic construct
  VARIANT 2: note = Aib / alpha-aminoisobutyric acid
  MOD_RES 76: note = C-terminal amide
YXEGTFTSDYSILLEEIAAREFIEWLLAGGPSSGAGQAPGQAPGASELSTAQTQRLSAELHKLATEPRTETGSGSP

SEQ ID NO: 211 - protein, 76 aa, synthetic construct
  VARIANT 2: note = Aib / alpha-aminoisobutyric acid
  MOD_RES 76: note = C-terminal amide
YXEGTFTSDYSILLEEIAAREFIEWLLAGGPSSGAGQAPGEAPGASHLSTAQTQRLSAELHKLATEPRTETGSGSP

SEQ ID NO: 212 - protein, 76 aa, synthetic construct
  VARIANT 2: note = Aib / alpha-aminoisobutyric acid
  MOD_RES 76: note = C-terminal amide
YXEGTFTSDYSILLEEIAAREFIEWLLAGGPSSEAGQAPGQAPGASHLSTAQTQRLSAELHKLATEPRTETGSGSP

SEQ ID NO: 213 - protein, 76 aa, synthetic construct
  VARIANT 2: note = Aib / alpha-aminoisobutyric acid
  MOD_RES 76: note = C-terminal amide
YXEGTFTSDYSILLEEIAAREFIEWLLAGGPSSGAGQAPGEAPGASELSTAQTQRLSAELHKLATLPRTETGSGSP

SEQ ID NO: 214 - protein, 76 aa, synthetic construct
  VARIANT 2: note = Aib / alpha-aminoisobutyric acid
  MOD_RES 76: note = C-terminal amide
YXEGTFTSDYSILLEEIAAREFIEWLLAGGPSSEAGQAPGQAPGASELSTAQTQRLSAELHKLATLPRTETGSGSP

SEQ ID NO: 215 - protein, 76 aa, synthetic construct
  VARIANT 2: note = Aib / alpha-aminoisobutyric acid
  MOD_RES 76: note = C-terminal amide
YXEGTFTSDYSILLEEIAAREFIEWLLAGGPSSEAGQAPGEAPGASHLSTAQTQRLSAELHKLATLPRTETGSGSP

SEQ ID NO: 216 - protein, 76 aa, synthetic construct
  VARIANT 2: note = Aib / alpha-aminoisobutyric acid
  MOD_RES 76: note = C-terminal amide
YXEGTFTSDYSKLLEEIAAREFIEWLLAGGPSSGAGQAPGQAPGASELSTAALGRLSAELHELATLPRTETGSGSP

SEQ ID NO: 217 - protein, 76 aa, synthetic construct
  VARIANT 2: note = Aib / alpha-aminoisobutyric acid
  MOD_RES 76: note = C-terminal amide
YXEGTFTSDYSKLLEEIAAREFIEWLLAGGPSSGAGQAPGQAPGASELSTAALGRLSAELHQLATLPRTETGSGSP

SEQ ID NO: 218 - protein, 76 aa, synthetic construct
  VARIANT 2: note = Aib / alpha-aminoisobutyric acid
  MOD_RES 76: note = C-terminal amide
YXEGTFTSDYSKLLEEIAAREFIEWLLAGGPSSGAGQAPGEAPGASELSTAALGRLSAELHQLATLPRTETGSGSP

SEQ ID NO: 219 - protein, 76 aa, synthetic construct
  VARIANT 2: note = Aib / alpha-aminoisobutyric acid
  MOD_RES 76: note = C-terminal amide
YXEGTFTSDYSKLLEEIAAREFIEWLLAGGPSSGAGQAPGEAPGASELSTAALGRLSAELHQLATEPRTETGSGSP

SEQ ID NO: 220 - protein, 76 aa, synthetic construct
  VARIANT 2: note = Aib / alpha-aminoisobutyric acid
  MOD_RES 76: note = C-terminal amide
YXEGTFTSDYSKLLEEIAAREFIEWLLAGGPSSGAGQAPGEAPGASELSTAQLGRLSAELHQLATEPRTETGSGSP

SEQ ID NO: 221 - protein, 76 aa, synthetic construct
  VARIANT 2: note = Aib / alpha-aminoisobutyric acid
  MOD_RES 76: note = C-terminal amide
YXEGTFTSDYSKLLEEIAAREFIEWLLAGGPSSGAGQAPGEAPGASELSTAQTGRLSAELHQLATEPRTETGSGSP

SEQ ID NO: 222 - protein, 76 aa, synthetic construct
  VARIANT 2: note = Aib / alpha-aminoisobutyric acid
  MOD_RES 76: note = C-terminal amide
YXEGTFTSDYSKLLEEIAAREFIEWLLAGGPSSGAGQAPGEAPGASELSTAQTQRLSAELHQLATEPRTETGSGSP

SEQ ID NO: 223 - protein, 68 aa, synthetic construct
  VARIANT 2: note = Aib / alpha-aminoisobutyric acid
  MOD_RES 68: note = C-terminal amide
YXEGTFTSDYSILLEEIAAREFIEWLLAGGPSSGAGASELSTAQTQRLSAELHKLATLPRTETGSGSP

SEQ ID NO: 224 - protein, 68 aa, synthetic construct
  VARIANT 2: note = Aib / alpha-aminoisobutyric acid
  MOD_RES 68: note = C-terminal amide
YXEGTFTSDYSILLEEIAAREFIEWLLAGGPSSEAGASHLSTAQTQRLSAELHKLATLPRTETGSGSP

SEQ ID NO: 225 - protein, 68 aa, synthetic construct
  VARIANT 2: note = Aib / alpha-aminoisobutyric acid
  MOD_RES 68: note = C-terminal amide
YXEGTFTSDYSILLEEIAAREFIEWLLAGGPSSEAGASELSTAQTQRLSAELHKLATLPRTETGSGSP

SEQ ID NO: 226 - protein, 68 aa, synthetic construct
  VARIANT 2: note = Aib / alpha-aminoisobutyric acid
  MOD_RES 68: note = C-terminal amide
YXEGTFTSDYSILLEEIAAREFIEWLLAGGPSSGAGASHLSTAQTQRLSAELHKLATEPRTETGSGSP

SEQ ID NO: 227 - protein, 68 aa, synthetic construct
  VARIANT 2: note = Aib / alpha-aminoisobutyric acid
  MOD_RES 68: note = C-terminal amide
YXEGTFTSDYSILLEEIAAREFIEWLLAGGPSSGAGASQLSTAQTQRLSAELHKLATEPRTETGSGSP

SEQ ID NO: 228 - protein, 68 aa, synthetic construct
  VARIANT 2: note = Aib / alpha-aminoisobutyric acid
  MOD_RES 68: note = C-terminal amide
YXEGTFTSDYSILLEEIAAREFIEWLLAGGPSSEAGASQLSTAQTQRLSAELHKLATLPRTETGSGSP

SEQ ID NO: 229 - protein, 68 aa, synthetic construct
  VARIANT 2: note = Aib / alpha-aminoisobutyric acid
  MOD_RES 68: note = C-terminal amide
YXEGTFTSDYSILLEEIAAREFIEWLLAGGPSSEAGASQLSTAQTQRLSAELHKLATEPRTETGSGSP

SEQ ID NO: 230 - protein, 68 aa, synthetic construct
  VARIANT 2: note = Aib / alpha-aminoisobutyric acid
  MOD_RES 68: note = C-terminal amide
YXEGTFTSDYSKLLEEIAAREFIEWLLAGGPSSGAGASELSTAQLGRLSAELHQLATLPRTETGSGSP

SEQ ID NO: 231 - protein, 68 aa, synthetic construct
  VARIANT 2: note = Aib / alpha-aminoisobutyric acid
  MOD_RES 68: note = C-terminal amide
YXEGTFTSDYSKLLEEIAAREFIEWLLAGGPSSGAGASHLSTAQLGRLSAELHQLATEPRTETGSGSP

SEQ ID NO: 232 - protein, 68 aa, synthetic construct
  VARIANT 2: note = Aib / alpha-aminoisobutyric acid
  MOD_RES 68: note = C-terminal amide
YXEGTFTSDYSKLLEEIAAREFIEWLLAGGPSSEAGASHLSTAQLGRLSAELHQLATEPRTETGSGSP

SEQ ID NO: 233 - protein, 68 aa, synthetic construct
  VARIANT 2: note = Aib / alpha-aminoisobutyric acid
  MOD_RES 68: note = C-terminal amide
YXEGTFTSDYSKLLEEIAAREFIEWLLAGGPSSGAGASELSTAQLGRLSAELHQLATEPRTETGSGSP

SEQ ID NO: 234 - protein, 68 aa, synthetic construct
  VARIANT 2: note = Aib / alpha-aminoisobutyric acid
  MOD_RES 68: note = C-terminal amide
YXEGTFTSDYSKLLEEIAAREFIEWLLAGGPSSEAGASELSTAQLGRLSAELHQLATLPRTETGSGSP

SEQ ID NO: 235 - protein, 68 aa, synthetic construct
  VARIANT 2: note = Aib / alpha-aminoisobutyric acid
  MOD_RES 68: note = C-terminal amide
YXEGTFTSDYSKLLEEIAAREFIEWLLAGGPSSEAGASELSTAQLGRLSAELHQLATEPRTETGSGSP

SEQ ID NO: 236 - protein, 76 aa, synthetic construct
  VARIANT 2: note = Aib / alpha-aminoisobutyric acid
  MOD_RES 76: note = C-terminal amide
YXEGTFTSDYSILLEEIAAREFIEWLLAGGPSSGAGEAPGEAPGASHLSTAQTQRLSAELHKLATLPRTETGSGSP

SEQ ID NO: 237 - protein, 76 aa, synthetic construct
  VARIANT 2: note = Aib / alpha-aminoisobutyric acid
  MOD_RES 76: note = C-terminal amide
YXEGTFTSDYSILLEEIAAREFIEWLIAGGPSSGAGEAPGQAPGASQLSTAQTQRLSAELHKLATLPRTETGSGSP

SEQ ID NO: 238 - protein, 76 aa, synthetic construct
  VARIANT 2: note = Aib / alpha-aminoisobutyric acid
  MOD_RES 76: note = C-terminal amide
YXEGTFTSDYSILLEEIAAREFIEWLIAGGPSSGAGEAPGQAPGASELSTAQTQRLSAELHKLATLPRTETGSGSP

SEQ ID NO: 239 - protein, 68 aa, synthetic construct
  VARIANT 2: note = Aib / alpha-aminoisobutyric acid
  MOD_RES 68: note = C-terminal amide
YXEGTFTSDYSILLEEIAAQEFIEWLLQGGPSSGAGASELSTAQTQRLSAELHKLATLPRTETGSGSP

SEQ ID NO: 240 - protein, 76 aa, synthetic construct
  VARIANT 2: note = Aib / alpha-aminoisobutyric acid
  MOD_RES 76: note = C-terminal amide
YXEGTFTSDYSILLEEIAAQEFIEWLLQGGPSSGAGEAPGQAPGASELSTAQTQRLSAELHKLATLPRTETGSGSP

SEQ ID NO: 241 - protein, 76 aa, synthetic construct
  VARIANT 2: note = Aib / alpha-aminoisobutyric acid
  MOD_RES 76: note = C-terminal amide
YXEGTFTSDYSILLEEIAAREFIEWLLAGGPSSGAGQAPGEAPLASHLSTAQTQRLSAELHKLATLPRTETGSGSP

SEQ ID NO: 242 - protein, 76 aa, synthetic construct
  VARIANT 2: note = Aib / alpha-aminoisobutyric acid
  MOD_RES 76: note = C-terminal amide
YXEGTFTSDYSILLEEIAAREFIEWLLAGGPSSGAGQAPGQAPLASHLSTAQTQRLSAELHKLATEPRTETGSGSP